(19) United States
(12) Patent Application Publication
(10) Pub. No.: US 2022/0241358 A1
(43) Pub. Date: Aug. 4, 2022

(54) APMV AND USES THEREOF FOR THE TREATMENT OF CANCER

(71) Applicant: ICAHN SCHOOL OF MEDICINE AT MOUNT SINAI, New York, NY (US)

(72) Inventors: Adolfo GARCIA-SASTRE, New York, NY (US); Peter PALESE, New York, NY (US); Sara CUADRADO CASTAÑO, New York, NY (US)

(73) Assignee: ICAHN SCHOOL OF MEDICINE AT MOUNT SINAI, New York, NY (US)

(21) Appl. No.: 17/527,903

(22) Filed: Nov. 16, 2021

Related U.S. Application Data
(63) Continuation of application No. 16/645,378, filed on Mar. 6, 2020, now abandoned.
(60) Provisional application No. 62/697,944, filed on Jul. 13, 2018.

(51) Int. Cl. A61K 35/768; A61P 35/00; A61K 38/17; C12N 7/00; C12N 15/86; A61K 48/00

(52) U.S. Cl. CPC A61K 35/768; A61P 35/00; A61K 38/1774; C07K 16/2818; C12N 15/86; A61K 48/00; C12N 7/00

(57) ABSTRACT

In one aspect, provided herein are naturally occurring and recombinantly produced avian paramyxovirus (APMV) (e.g., an APMV-2, APMV-3, APMV-4, APMV-6, APMV-7, APMV-8, and APMV-9 strain) and uses of such APMV for the treatment of cancer. In particular, provided herein are methods for treating cancer comprising administering a naturally occurring or recombinantly produced APMV-4 strain to a subject in need thereof. In another aspect, provided herein are recombinant APMV comprising a packaged genome, wherein the packaged genome comprises a transgene. In particular, described herein are recombinant APMV (e g., APMV-2, APMV-3, APMV-4, APMV-6, APMV-7, APMV-8, and APMV-9). In another aspect, provided herein are methods for treating cancer comprising administering a recombinant APMV (e g., APMV-2, APMV-3, APMV-4, APMV-6, APMV-7, APMV-8, and APMV-9) to a subject in need thereof, wherein the recombinant APMV comprises a packaged genome comprising a transgene. In particular, provided herein are methods for treating cancer comprising administering a recombinant APMV-4 to a subject in need thereof, wherein the recombinant APMV-4 comprises a packaged genome comprising a transgene. In specific aspects, the use of APMV serotypes other than APMV-1 (such as described herein, in particular AMPV-4) to treat cancer is based, in part, on the similar or enhanced in vivo anti-tumor activities when compared to oncolytic NDV La Sota-L289A strain.

This application claims the benefit of priority of U.S. provisional patent application No. 62/697,944, filed Jul. 13, 2018, which is incorporated by reference herein in its entirety.

The instant application contains a Sequence Listing which has been submitted electronically in ASCII format and is hereby incorporated by reference in its entirety. Said ASCII copy, created on Jul. 9, 2019, is named 6923-282-228_SL.txt and is 322,198 bytes in size.

### 1. INTRODUCTION

In one aspect, provided herein are naturally occurring and recombinantly produced avian paramyxovirus (APMV) (e.g., an APMV-2, APMV-3, APMV-4, APMV-6, APMV-7, APMV-8, and APMV-9 strain) and uses of such APMV for the treatment of cancer. In particular, provided herein are methods for treating cancer comprising administering a naturally occurring or recombinantly produced APMV-4 strain to a subject in need thereof. In another aspect, provided herein are recombinant APMVs comprising a packaged genome, wherein the packaged genome comprises a transgene. In particular, described herein are recombinant APMV (e.g., APMV-2, APMV-3, APMV-4, APMV-6, APMV-7, APMV-8, and APMV-9). In another aspect, provided herein are methods for treating cancer comprising administering a recombinant APMV (e.g., APMV-2, APMV-3, APMV-4, APMV-6, APMV-7, APMV-8, and APMV-9) to a subject in need thereof, wherein the recombinant APMV comprises a packaged genome comprising a transgene. In particular, provided herein are methods for treating cancer comprising administering a recombinant APMV-4 to a subject in need thereof, wherein the recombinant APMV-4 comprises a packaged genome comprising a transgene. In specific aspects, the use of APMV serotypes other than APMV-1 (such as described herein, in particular AMPV-4) to treat cancer is based, in part, on the similar or enhanced in vivo anti-tumor activities when compared to oncolytic NDV La Sota-L289A strain.

### 2. BACKGROUND

The family Paramyxoviridae includes important respiratory and systemic pathogens of humans (mumps, measles, human parainfluenza viruses) and animals (Sendai, canine disempter viruses, Newcastle disease viruses), including several zoonotic emerging viruses (Hendra and Nipah viruses). Paramyxoviruses are enveloped pleomorphic viruses containing a non-segmented, negative-sense, single stranded RNA genome which encodes 6-10 viral genes and that replicate in the cytoplasm of the host cell. All the paramyxoviruses isolated from avian species, with the only exception of the avian metapneumovirus, are classified into the genus Avulavirus (1). With a size range of 14900-17000 nucleotides, the genome of all avian avulaviruses encodes 6 structural proteins involved in viral replication cycle: the nucleoprotein (NP), the phosphoprotein (P) and the large polymerase protein (L) are, in association with the viral RNA, the components of the ribonucleotide protein complex (RNP). The RNP exerts dual function acting as a nucleocapside (i) and as the replication machinery of the virus (ii). The matrix protein (M) assembles between the viral envelope and the nucleocapside and participates actively during the processes of virus assembly and budding (2). The hemagglutinin-neuraminidase (HN) and fusion (F) glycoproteins, in conjunction with a host-derived lipid bilayer constitute the external envelope of the virus.

The Avulavirus genus is further divided into different serotypes based on hemagglutination inhibition (HI) and neuraminidase inhibition (NI) assays (3, 4). The most recent taxonomic revision of the group recognizes 13 serotypes of avian avulaviruses (Table 1), noted as APMVs (from avian paramyxovirus).

TABLE 1
Review of the Accepted Serotypes Included Within the Avulavirus Gene

| | | | PATH. | PLACE OF | |
|---|---|---|---|---|---|
| SEROTYPE | YEAR | HOST | CHICKENS | ISOLATION | REF |
| APMV-1 | 1926 | Chicken | Avirulent/Virulent | Java (Indonesia), | [61] |
| | | | | Newcastle upon Tyne | |
| | | | | (England) | |
| APMV-2 | 1956 | Chicken and turkey | Avirulent/Virulent | Yucaipa and California | [62] |
| | | | | (USA) England and | |
| | | | | Kenya | |
| APMV-3 | 1967 | Turkey and parakeet | Avirulent | Ontario, | [63-65] |
| | | | | Wisconsin(USA) | |
| | | | | England, France and the | |
| | | | | Netherlands | |
| APMV-4 | 1976 | Wild Duck, chicken, | Avirulent/Virulent | Mississippi, Hong- | [66, 67] |
| | | geese and mallard duck | | Kong, Korea and South | |
| | | | | Africa | |
| APMV-5 | 1974 | Budgerigar | Avirulent/Virulent | Japan and UK | [68, 69] |
| APMV-6 | 1977 | Domestic duck, geese, | Avirulent | Hong-Kong, Taiwan, | [70-71] |
| | | turkey and mallard duck | | Italy and New Zealand | |
| APMV-7 | 1975 | Hunter-killed dove, | Virulent | Tennessee (USA) | [72-74] |
| | | turkey and ostrich | | | |
| APMV-8 | 1976 | Feral Canadian goose | Avirulent | USA and Japan | [75, 76] |
| | | and pintail | | | |
| APMV-9 | 1978 | Domestic and feral duck | Virulent | New York (USA) and | [77-78] |
| | | | | Italy | |
| APMV-10 | 2007 | Rockhopper Penguin | Avirulent | Falkland Islands | [79] |
| APMV-11 | 2010 | Common snipe | Avirulent | France | [80] |
| APMV-12 | 2005 | Wigeon | Avirulent | Italy | [81] |
| APMV-13 | 2000 | Geese | N.D | Shimane (Japan) and | [82-83] |
| | | | | Kazakhstan | |

APMVs have been isolated from a wide-range of domestic and wild birds. Clinical signs of the infection vary from asymptomatic to high morbidity and mortality in a strain-specific and host-dependent manner (5). Avian avulavirus 1 (APMV-1), commonly known as Newcastle disease virus (NDV), is the only well-characterized serotype due to the high mortality rates and economic losses caused by virulent strains in the poultry industry (6, 7). Regardless of the devastating impact of highly pathogenic strains, Newcastle disease can be controlled by the prophylactic administration of live attenuated and/or killed virus vaccines (8, 9). APMV-1 strains have been classified into three different pathotypes, velogenic (highly virulent), mesogenic (intermediate virulence) and lentogenic (low-virulence or avirulent), in accordance with the severity of the clinical signs displayed by affected chickens (10). Despite its prevalence and worldwide distribution, APMV-1 viruses do not represent a human threat. Occasional human infections are restricted to direct contact with sick birds and resolved with mild flu-like symptoms or conjunctivitis (11). Reported APMV-1 infections in mammals have demonstrated that these avian viruses are neither capable to establish persistent infection nor to counteract the antiviral innate response in mammalian cells (12-14). Furthermore, different strains of NDV have shown to act as strong stimulators of humoral and cellular immune responses at both the local and systemic levels (15-19). Reverse genetics systems have been developed that allow the genetic manipulation of the NDV genome (20-22). Based on the safety and immunostimulatory properties displayed by APMV-1 strains in mammals, several recombinant NDV vaccine strains have been used as vaccine vectors in poultry and mammals to express antigens of different pathogens (22-28).

Over the past three decades there has been an increased interest in the use of AMPV-1 as an antineoplastic agent (29). The inherent anti-tumor capacity of APMV-1 strains combines two properties that define an oncolytic virus (OV): induction of specific tumor cell death (30) accompanied by the elicitation of antitumor immunity and long-term tumor remission (31-34). From the first reports in the 60′s about the anti-tumor potential of NDV (35, 36) until now, different APMV-1 strains have directly been applied as anti-cancer therapy in animal models and/or cancer patients by different routes (intra-tumoral, locoregional or systemic) (37-39) or been used as viral oncolysates (40, 41), live cell tumor vaccines (NDV-ATV) (34, 42-46), or DC vaccines pulsed with viral oncolysates (47-49) to treat tumors. Although AMPV-1 has been in clinical studies to examine its anti-cancer effects, it has not been approved for the treatment of any human cancers.

Nowadays, multiple research groups work towards the development of more efficient AMPV-1 -based anti-tumor strategies that could overcome tumor-associated mechanisms of resistance (50-59). For example, recent studies have shown that AMPV-1 ultimately induces the upregulation of PD-L1 expression in tumor cells and tumor-infiltrating immune cells (Zamarin et al., 2018, J. Clin. Invest. 128: 1413-1428), providing a strong rationale for clinical exploration of combinations of immunoregulatory antibodies.

In contrast to what is known about APMV-1 strains, there is limited information associated with the biology of other avian avulavirus serotypes. Although the anti-tumor potential of NDV has been tested, no NDV-based anti-tumor therapy has been approved for the treatment of cancer. Thus, there is need for therapies for the treatment of cancer.

### 3. SUMMARY

In one aspect, provided herein are naturally occurring and recombinantly produced avian paramyxovirus (APMV) (e.g., an APMV-2, APMV-3, APMV-4, APMV-6, APMV-7, APMV-8, and APMV-9 strain) and uses of such APMV for the treatment of cancer. In a specific embodiment, the APMV (e.g., APMV-4) is administered to the human subject intratumorally or intravenously. In another specific embodiment, the APMV (e.g., APMV-4) is administered at a dose of 10⁶ to 10¹² plaque-forming units (pfu).

The use of APMV serotypes other than APMV-1 to treat cancer is based, in part, on the similar or enhanced in vivo anti-tumor activities when compared to oncolytic NDV La Sota-L289A strain. In particular, the use of APMV-4 to treat cancer is based, in part, on the statistically significant anti-tumor activity observed in different animal models for various tumors. See Section 6 infra.

In a specific embodiment, provided herein is a method for treating cancer, comprising administering to a human subject in need thereof a naturally occurring APMV (e.g., an APMV-2, APMV-3, APMV-4, APMV-6, APMV-7, APMV-8, and APMV-9 strain), wherein the APMV has an intracerebral pathogenicity index in day-old chicks of the Gallus gallus species of less than 0.7. In another specific embodiment, provided herein is a method for treating cancer, comprising administering to a human subject in need thereof a recombinant APMV (e.g., an APMV-2, APMV-3, APMV-4, APMV-6, APMV-7, APMV-8, and APMV-9 strain), wherein the recombinant APMV has an intracerebral pathogenicity index in day-old chicks of the Gallus gallus species of less than 0.7. In a specific embodiment, the APMV (e.g., an APMV-2, APMV-3, APMV-4, APMV-6, APMV-7, APMV-8, and APMV-9 strain) is administered to the human subject intratumorally or intravenously. In another specific embodiment, the APMV is administered at a dose of 10⁶ to 10¹² pfu. In some embodiments, the method for treating cancer further comprises administering the subject a checkpoint inhibitor. In certain embodiments, the method for treating cancer further comprises administering the subject a monoclonal antibody that specifically binds to PD-1 and blocks the binding of PD-1 to PD-L1 and PD-L2.

In a specific embodiment, provided herein is a method for treating cancer, comprising administering to a human subject in need thereof a naturally occurring APMV-4, wherein the APMV-4 has an intracerebral pathogenicity index in day-old chicks of the Gallus gallus species of less than 0.7. In another specific embodiment, provided herein is a method for treating cancer, comprising administering to a human subject in need thereof a recombinant APMV-4, wherein the recombinant APMV-4 has an intracerebral pathogenicity index in day-old chicks of the Gallus gallus species of less than 0.7. In a specific embodiment, the APMV-4 is administered to the human subject intratumorally or intravenously. In another specific embodiment, the APMV-4 is administered at a dose of 10⁶ to 10¹² pfu. In some embodiments, the method for treating cancer further comprises administering the subject a checkpoint inhibitor. In certain embodiments, the method for treating cancer further comprises administering the subject a monoclonal antibody that specifically binds to PD-1 and blocks the binding of PD-1 to PD-L1 and PD-L2.

In certain embodiments, the APMV-4 that is administered to a subject in accordance with the methods described herein is an APMV-4 that when administered to a B16-F10 syngeneic murine melanoma model decreases tumor growth and increases survival of the B16-F10 syngeneic murine melanoma model as compared to tumor growth and survival in a B16-F10 syngeneic murine melanoma model administered phosphate buffered saline (PBS). In some embodiments, the APMV-4 that is administered to a subject in accordance with the methods described herein is an APMV-4 that when administered to a B16-F10 syngeneic murine melanoma model results in a greater decrease in tumor growth and a longer survival time of the B16-F10 syngeneic murine melanoma model as compared to tumor growth and survival time in a B16-F10 syngeneic murine melanoma model administered a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, the packaged genome of the modified NDV LaSota comprises the negative sense RNA transcribed from the cDNA sequence set forth in SEQ ID NO:13.

In certain embodiments, the APMV-4 that is administered to a subject in accordance with the methods described herein is an APMV-4 that when administered to a BALBc syngeneic murine colon carcinoma tumor model decreases tumor growth and increases survival of the BALBc syngeneic murine colon carcinoma tumor model as compared to tumor growth and survival of a BALBc syngeneic murine colon carcinoma tumor model administered PBS. In some embodiments, the APMV-4 that is administered to a subject in accordance with the methods described herein is an APMV-4 that when administered to a BALBc syngeneic murine colon carcinoma tumor model results in a greater decrease in tumor growth and a longer survival time of the BALBc syngeneic murine colon carcinoma tumor model as compared to tumor growth and survival time in a BALBc syngeneic murine colon carcinoma tumor model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, the packaged genome of the modified NDV LaSota comprises the negative sense RNA transcribed from the cDNA sequence set forth in SEQ ID NO:13.

In certain embodiments, the APMV-4 that is administered to a subject in accordance with the methods described herein is an APMV-4 that when administered to a C57BL/6 syngeneic lung carcinoma tumor model decreases tumor growth and increases survival of the C57BL/6 syngeneic murine lung carcinoma tumor model as compared to tumor growth and survival in a C57BL/6 syngeneic murine lung carcinoma tumor model administered phosphate buffered saline (PBS). In some embodiments, the APMV-4 that is administered to a subject in accordance with the methods described herein is an APMV-4 that when administered to a C57BL/6 syngeneic murine lung carcinoma tumor model results in a greater decrease in tumor growth and a longer survival time of the C57BL/6 syngeneic murine lung carcinoma tumor model as compared to tumor growth and survival time in a C57BL/6 syngeneic murine lung carcinoma tumor model administered a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, the packaged genome of the modified NDV LaSota comprises the negative sense RNA transcribed from the cDNA sequence set forth in SEQ ID NO:13.

In a specific embodiment, provided herein is a method for treating cancer, comprising administering to a human subject in need thereof a naturally occurring APMV-8, wherein the APMV-8 has an intracerebral pathogenicity index in day-old chicks of the Gallus gallus species of less than 0.7. In a specific embodiment, provided herein is a method for treating cancer, comprising administering to a human subject in need thereof a recombinant APMV-8, wherein the recombinant APMV-8 has an intracerebral pathogenicity index in day-old chicks of the Gallus gallus species of less than 0.7. In a particular embodiment, the APMV-8 is APMV-8 Goose/Delaware/1053/1976. In certain embodiments, the APMV-8 that is administered to a subject in accordance with the methods described herein is an APMV-8 that decreases tumor growth and increases survival in a BALBc syngeneic murine colon carcinoma tumor model as compared to tumor growth and survival in a BALBc syngeneic murine colon carcinoma tumor model administered PBS. In some embodiment, the APMV-8 that is administered to a subject in accordance with the methods described herein is an APMV-8 that results in a greater decrease in tumor growth and a longer survival time in a BALBc syngeneic murine colon carcinoma tumor model as compared to tumor growth and survival time in a BALBc syngeneic murine colon carcinoma tumor model administered a genetically modified NDV, wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, the packaged genome of the modified NDV LaSota comprises the negative sense RNA transcribed from the cDNA sequence set forth in SEQ ID NO:13.

In another aspect, provided herein is a recombinant APMV (e.g., an APMV-2, APMV-3, APMV-4, APMV-6, APMV-7, APMV-8, and APMV-9 strain) comprising a packaged genome comprising a transgene encoding a heterologous sequence. In a specific embodiment, provided herein is a recombinant APMV (e.g., an APMV-2, APMV-3, APMV-4, APMV-6, APMV-7, APMV-8, and APMV-9 strain) comprising a packaged genome comprising a transgene encoding a cytokine, interleukin-15 (IL-15) receptor alpha (IL-15Ra)-IL-15, human papillomavirus (HPV)-16 E6 protein or HPV-16 E7 protein. In certain embodiments, the APMV (e.g., an APMV-2, APMV-3, APMV-4, APMV-6, APMV-7, APMV-8, and APMV-9 strain) has an intracerebral pathogenicity index in day-old chicks of the Gallus gallus species of less than 0.7. In a specific embodiment, a recombinant APMV described herein comprises an APMV-7 or APMV-8 backbone. In another specific embodiment, a recombinant APMV described herein comprises the APMV-8 Goose/Delaware/1053/1976 backbone. In another specific embodiment, a recombinant APMV described herein comprises the APMV-7 Dove/Tennessee/4/1975 backbone. In another specific embodiment, the recombinant APMV comprises an APMV-4 backbone. In a specific embodiment, a recombinant APMV described herein comprises an APMV-4 Duck/Hong Kong/D3/1975 strain backbone, an APMV-4 Duck/China/G302/2012 strain backbone, APMV4/mallard/Belgium/15129/07 strain backbone; APMV4Uriah-aalge/Russia/Tyuleniy_Island/115/2015 strain backbone, APMV4/Egyptian goose/South Africa/NJ468/2010 strain backbone, or APMV4/duck/Delaware/549227/2010 strain backbone. In a specific embodiment, the transgene is inserted between two transcription units of the APMV packaged genome (e.g., APMV M and P transcription units). In one embodiment, the cytokine is interleukin-12 (IL-12). In a specific embodiment, the IL-12 is encoded by a nucleotide sequence comprising the nucleotide sequence of SEQ ID NO:16 or 17. In another embodiment, the cytokine is interleukin-2 (IL-2). In a specific embodiment, the IL-2 is encoded by a nucleotide sequence comprising the nucleotide sequence of SEQ ID NO:15. In another embodiment, the cytokine is granulocyte-macrophage colony-stimulating factor (GM-CSF). In a specific embodiment, the GM-CSF is encoded by a nucleotide sequence comprising the nucleotide sequence of SEQ ID NO:21. In another embodiment, the transgene comprises a nucleotide sequence encoding IL-15Ra-IL15. In a specific embodiment, the nucleotide sequence encoding IL-15Ra-IL-15 comprises the negative sense RNA transcribed from the nucleotide sequence of SEQ ID NO:18. In another embodiment, the transgene comprises a nucleotide sequence encoding HPV-16 E6 protein. In a specific embodiment, the nucleotide sequence encoding the HPV-16 E6 protein comprises the negative sense RNA transcribed from the nucleotide sequence of SEQ ID NO:19. In another embodiment, the transgene comprises a nucleotide sequence encoding HPV-16 E7 protein. In a specific embodiment, the nucleotide sequence encoding the HPV-16 E7 protein comprises the negative sense RNA transcribed from the nucleotide sequence of SEQ ID NO:20.

In a specific embodiment, provided herein is a recombinant APMV-4 comprising a packaged genome comprising a transgene encoding a cytokine, IL-15Ra-IL-15, HPV-16 E6 protein or HPV-16 E7 protein, and wherein the APMV-4 has an intracerebral pathogenicity index in day-old chicks of the Gallus gallus species of less than 0.7. In a specific embodiment, the transgene is inserted between two transcription units of the APMV-4 packaged genome (e.g., APMV-4 M and P transcription units). In one embodiment, the cytokine is IL-12. In a specific embodiment, the IL-12 is encoded by a nucleotide sequence comprising the nucleotide sequence of SEQ ID NO:16 or 17. In another embodiment, the cytokine is IL-2. In a specific embodiment, the IL-2 is encoded by a nucleotide sequence comprising the nucleotide sequence of SEQ ID NO:15. In another embodiment, the cytokine is GM-CSF. In a specific embodiment, the GM-CSF is encoded by a nucleotide sequence comprising the nucleotide sequence of SEQ ID NO:21. In another embodiment, the transgene comprises a nucleotide sequence encoding IL-15Ra-IL15. In a specific embodiment, the nucleotide sequence encoding IL-15Ra-IL-15 comprises the negative sense RNA transcribed from the nucleotide sequence of SEQ ID NO:18. In another embodiment, the transgene comprises a nucleotide sequence encoding HPV-16 E6 protein. In a specific embodiment, the nucleotide sequence encoding the HPV-16 E6 protein comprises the negative sense RNA transcribed from the nucleotide sequence of SEQ ID NO:19. In another embodiment, the transgene comprises a nucleotide sequence encoding HPV-16 E7 protein. In a specific embodiment, the nucleotide sequence encoding the HPV-16 E7 protein comprises the negative sense RNA transcribed from the nucleotide sequence of SEQ ID NO:20.

In another specific embodiment, provided herein is a recombinant APMV-4 comprising a packaged genome comprising a transgene encoding IL-12. In a specific embodiment, the APMV-4 has an intracerebral pathogenicity index in day-old chicks of the Gallus gallus species of less than 0.7. In another specific embodiment, the packaged genome of the APMV-4 comprises the negative sense RNA transcribed from the cDNA sequence set forth in SEQ ID NO:14.

In a specific embodiment, a recombinant APMV-4 described herein comprises an APMV-4 Duck/Hong Kong/D3/1975 strain backbone. In another embodiment, a recombinant APMV-4 described herein comprises an APMV-4 Duck/China/G302/2012 strain backbone, APMV4/mallard/Belgium/15129/07 strain backbone; APMV4Uriah-aalge/Russia/Tyuleniy_Island/115/2015 strain backbone, APMV4/Egyptian goose/South Africa/NJ468/2010 strain backbone, or APMV4/duck/Delaware/549227/2010 strain backbone.

In specific embodiments, provided herein is a method for treating cancer, comprising administering to a human subject in need thereof a recombinant APMV described herein. In certain embodiments, a recombinant APMV described herein is administered to the human subject intratumorally or intravenously. In some embodiments, a recombinant APMV described herein is administered at a dose of 10⁶ to 10¹² pfu. In a specific embodiment, a recombinant APMV described herein comprises an APMV-4 or APMV-8 backbone. In some embodiments, the method for treating cancer further comprises administering the subject a checkpoint inhibitor. In certain embodiments, the method for treating cancer further comprises administering the subject a monoclonal antibody that specifically binds to PD-1 and blocks the binding of PD-1 to PD-L1 and PD-L2.

In certain embodiments, the cancer treated in accordance with the methods described herein is melanoma, lung carcinoma, colon carcinoma, B-cell lymphoma, T-cell lymphoma, or breast cancer. In a specific embodiment, the cancer treated in accordance with the methods described herein is metastatic. In another specific embodiment, the cancer treated in accordance with the methods described herein is unresectable.

### 3.1 Terminology

As used herein, the term “about” or “approximately” when used in conjunction with a number refers to any number within 1, 5 or 10% of the referenced number.

As used herein, the terms “antibody” and “antibodies” refer to molecules that contain an antigen-binding site, e.g., immunoglobulins. Antibodies include, but are not limited to, monoclonal antibodies, bispecific antibodies, multispecific antibodies, human antibodies, humanized antibodies, synthetic antibodies, chimeric antibodies, polyclonal antibodies, single domain antibodies, camelized antibodies, single-chain Fvs (scFv), single chain antibodies, Fab fragments, F(ab') fragments, disulfide-linked bispecific Fvs (sdFv), intrabodies, and anti-idiotypic (anti-Id) antibodies (including, e.g., anti-Id and anti-anti-Id antibodies to antibodies), and epitope-binding fragments of any of the above. In particular, antibodies include immunoglobulin molecules and immunologically active fragments of immunoglobulin molecules. Immunoglobulin molecules can be of any type (e.g., IgG, IgE, IgM, IgD, IgA and IgY), class (e.g., IgG1, IgG2, IgG3, IgG4, IgA1 and IgA2) or subclass. In a specific embodiment, an antibody is a human or humanized antibody. In another specific embodiment, an antibody is a monoclonal antibody or scFv. In certain embodiments, an antibody is a human or humanized monoclonal antibody or scFv. In other specific embodiments, the antibody is a bispecific antibody.

As used herein, the term “derivative” in the context of proteins or polypeptides includes: (a) a polypeptide that is at least 80%, 85%, 90%, 95%, 98%, or 99% or is 80% to 85%, 80% to 90%, 80% to 95%, 90% to 95%, 85% to 99%, or 95% to 99% identical to a native polypeptide; (b) a polypeptide encoded by a nucleic acid sequence that is at least 80%, 85%, 90%, 95%, 98%, or 99% or is 80% to 85%, 80% to 90%, 80% to 95%, 90% to 95%, 85% to 99%, or 95% to 99% identical to a nucleic acid sequence encoding a native polypeptide; (c) a polypeptide that contains 1, 2, 3, 4, 5, 6, 7, 8, 9, 10, 11, 12, 13, 14, 15, 16, 17, 18, 19, 20 or more, or 2 to 5, 2 to 10, 5 to 10, 5 to 15, 5 to 20, 10 to 15, or 15 to 20 amino acid mutations (i.e., any one or more, or all of an addition(s), deletion(s) or substitution(s)) relative to a native polypeptide; (d) a polypeptide encoded by nucleic acid sequence that can hybridize under high, moderate or typical stringency hybridization conditions to a nucleic acid sequence encoding a native polypeptide; (e) a polypeptide encoded by a nucleic acid sequence that can hybridize under high, moderate or typical stringency hybridization conditions to a nucleic acid sequence encoding a fragment of a native polypeptide of at least 10 contiguous amino acids, at least 12 contiguous amino acids, at least 15 contiguous amino acids, at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, at least 150 contiguous amino acids, or 10 to 20, 20 to 50, 25 to 75, 25 to 100, 25 to 150, 50 to 75, 50 to 100, 75 to 100, 50 to 150, 75 to 150, 100 to 150, or 100 to 200 contiguous amino acids; or (f) a fragment of a native polypeptide. Derivatives also include a polypeptide that comprises the amino acid sequence of a naturally occurring mature form of a mammalian polypeptide and a heterologous signal peptide amino acid sequence. In addition, derivatives include polypeptides that have been chemically modified by, e.g., glycosylation, acetylation, pegylation, phosphorylation, amidation, derivitization by known protecting/blocking groups, proteolytic cleavage, linkage to a cellular ligand or other protein moiety, etc. Further, derivatives include polypeptides comprising one or more non-classical amino acids. In one embodiment, a derivative is isolated. In specific embodiments, a derivative retains one or more functions of the native polypeptide from which it was derived.

As used herein, the term “elderly human” refers to a human 65 years or older.

As used herein, the term “fragment” in the context of a nucleotide sequence refers to a nucleotide sequence comprising a nucleic acid sequence of at least 5 contiguous nucleic acid bases, at least 10 contiguous nucleic acid bases, at least 15 contiguous nucleic acid bases, at least 20 contiguous nucleic acid bases, at least 25 contiguous nucleic acid bases, at least 40 contiguous nucleic acid bases, at least 50 contiguous nucleic acid bases, at least 60 contiguous nucleic acid bases, at least 70 contiguous nucleic acid bases, at least 80 contiguous nucleic acid bases, at least 90 contiguous nucleic acid bases, at least 100 contiguous nucleic acid bases, at least 125 contiguous nucleic acid bases, at least 150 contiguous nucleic acid bases, at least 175 contiguous nucleic acid bases, at least 200 contiguous nucleic acid bases, or at least 250 contiguous nucleic acid bases of the nucleotide sequence of the gene of interest. The nucleic acid may be RNA, DNA, or a chemically modified variant thereof.

As used herein, the term “fragment” is the context of a fragment of a proteinaceous agent (e.g., a protein or polypeptide) refers to a fragment that is composed of 8 or more contiguous amino acids, 10 or more contiguous amino acids, 15 or more contiguous amino acids, 20 or more contiguous amino acids, 25 or more contiguous amino acids, 50 or more contiguous amino acids, 75 or more contiguous amino acids, 100 or more contiguous amino acids, 150 or more contiguous amino acids, 200 or more contiguous amino acids, 10 to 150 contiguous amino acids, 10 to 200 contiguous amino acids, 10 to 250 contiguous amino acids, 10 to 300 contiguous amino acids, 50 to 100 contiguous amino acids, 50 to 150 contiguous amino acids, 50 to 200 contiguous amino acids, 50 to 250 contiguous amino acids or 50 to 300 contiguous amino acids of a proteinaceous agent.

As used herein, the term “heterologous” to refers an entity not found in nature to be associated with (e.g., encoded by, expressed by the genome of, or both) a naturally occurring APMV. In a specific embodiment, a heterologous sequence encodes a protein that is not found associated with naturally occurring APMV.

As used herein, the term “human adult” refers to a human that is 18 years or older.

As used herein, the term “human child” refers to a human that is 1 year to 18 years old.

As used herein, the term “human infant” refers to a newborn to 1-year-old year human.

As used herein, the term “human toddler” refers to a human that is 1 year to 3 years old.

As used herein, the term “in combination” in the context of the administration of (a) therapy(ies) to a subject, refers to the use of more than one therapy. The use of the term “in combination” does not restrict the order in which therapies are administered to a subject. A first therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject. For example, a recombinant APMV described herein may be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before) concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of another therapy.

As used herein, the phrases “interferon-deficient systems,” “interferon-deficient substrates,” “IFN deficient systems” or “IFN-deficient substrates” refer to systems, e.g., cells, cell lines and animals, such as mice, chickens, turkeys, rabbits, rats, horses etc., which do not produce one, two or more types of IFN, or do not produce any type of IFN, or produce low levels of one, two or more types of IFN, or produce low levels of any IFN (i.e., a reduction in any IFN expression of 5-10%, 10-20%, 20-30%, 30-40%, 40-50%, 50-60%, 60-70%, 70-80%, 80-90% or more when compared to IFN-competent systems under the same conditions), do not respond or respond less efficiently to one, two or more types of IFN, or do not respond to any type of IFN, have a delayed response to one, two or more types of IFN, and/or are deficient in the activity of antiviral genes induced by one, two or more types of IFN, or induced by any type of IFN.

As used herein, the phrase “multiplicity of infection” or “MOI” has its customary meaning. Generally, MOI is the average number of virus per infected cell. The MOI is determined by dividing the number of virus added (ml added×Pfu) by the number of cells added (ml added×cells/ml).

As used herein, the term “native” in the context of proteins or polypeptides refers to any naturally occurring amino acid sequence, including immature or precursor and mature forms of a protein. In a specific embodiment, the native polypeptide is a human protein or polypeptide.

As used herein, the term “naturally occurring” in the context of an APMV refers to an APMV found in nature, which is not modified by the hand of man. In other words, a naturally occurring APMV is not genetically engineered or otherwise altered by the hand of man.

As used herein, the terms “subject” or “patient” are used interchangeably. As used herein, the terms “subject” and “subjects” refers to an animal. In some embodiments, the subject is a mammal including a non-primate (e.g., a camel, donkey, zebra, bovine, horse, horse, cat, dog, rat, and mouse) and a primate (e.g., a monkey, chimpanzee, and a human). In some embodiments, the subject is a non-human mammal. In certain embodiments, the subject is a pet (e.g., dog or cat) or farm animal (e.g., a horse, pig or cow). In specific embodiments, the subject is a human. In certain embodiments, the mammal (e.g., human) is 4 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In specific embodiments, the subject is an animal that is not avian.

As used herein, the terms “therapies” and “therapy” can refer to any protocol(s), method(s), agent(s) or a combination thereof that can be used in the treatment cancer. In certain embodiments, the term “therapy” refers to an APMV described herein. In other embodiments, the term “therapy” refers to an agent that is not an APMV described herein.

### 4. BRIEF DESCRIPTION OF THE FIGURES

FIGS. 1A-1B. Infectivity and cytotoxicity of APMVs in a B16-F10 murine melanoma cancer cell line. FIG. 1A depicts microscopy images of B16-F10 murine melanoma cells infected by APMVs. Cells were infected at an MOI of 1 FFU/cell, fixed 20 hours after infection, and stained with polyclonal anti-APMV species-specific serum (red), polyclonal anti-NDV serum (green), and Hoechst for nuclear contrast. FIG. 1B depicts in vitro cytotoxicity. B16-F10 cells were infected at an MOI of 1 FFU/cell and their viability was determined by CellTiter-Fluor™ viability assay at 24 hours after infection. Bars represent mean values±standard deviation (SD) (n=3; **, P<0.01; ***, P<0.001; ****, P<0.0001).

FIGS. 2A-2C. Oncolytic capacity of APMVs in a syngenic murine melanoma tumor model. FIG. 2A depicts individual tumor growth curves. Each point represents tumor volume per mouse at the indicated time points. FIG. 2B depicts analysis of tumor growth rate. Points represent average of tumor volume per experimental group at the indicated time points. Error bars correspond to SD of each group. FIG. 2C depicts overall survival of treated B16-F10 tumor-bearing mice (*, P<0.03).

FIG. 3A-3D. Oncolytic capacity of APMVs in a syngenic murine colon carcinoma model. FIG. 3A depicts individual tumor growth curves. Each point represents tumor volume per mouse at the indicated time points. FIG. 3B represents analysis of the tumor growth rate. Each point represents tumor volume per treatment group at the indicated time points. FIG. 3C depicts overall survival of the treated CT26 tumor-bearing mice. FIG. 3D depicts overall survival of the treated CT26 tumor-bearing mice, where tumor-free survivors were re-challenged by intradermal injection of CT26 cells in the flank of the posterior left leg (contralateral).

FIGS. 4A-4C. Oncolytic capacity of APMV-4 in a syngenic murine lung carcinoma model. FIG. 4A depicts individual tumor growth curves. Each point represents tumor volume per mouse at the indicated time points. FIG. 4B represents analysis of the tumor growth rate. Points represent average tumor volume per experimental group at the indicated time point; right side: statistical analysis of control of tumor growth after third injection. Error bars correspond to SD of each group. FIG. 4C depicts overall survival of the treated TC-1 tumor-bearing mice (**, P<0.03).

### 5. DETAILED DESCRIPTION

### 5.1 Avian Paramyoxviruses

### 5.1.1 APMV

Any APMV-2, APMV-3, APMV-4, APMV-6, APMV-7, APMV-8, or APMV-9 strain may be serve, including, but not limited to, naturally-occurring strains, variants or mutants, mutagenized viruses, genetically engineered viruses, or a combination thereof may be used in the methods for treating cancer described herein. In certain embodiments, the APMV-2, APMV-3, APMV-4, APMV-6, APMV-7, APMV-8, or APMV-9 strain that is used in a method of treating cancer described herein is a lytic strain. In other embodiments, the APMV-2, APMV-3, APMV-4, APMV-6, APMV-7, APMV-8, or APMV-9 strain that is used in a method of treating cancer described herein is a non-lytic strain. In a specific embodiment, the APMV-2, APMV-3, APMV-4, APMV-6, APMV-7, APMV-8, or APMV-9 strain that is used in a method of treating cancer described herein is naturally occurring. In a specific embodiment, the APMV-2, APMV-3, APMV-4, APMV-6, APMV-7, APMV-8, or APMV-9 strain that is used in a method of treating cancer described herein is avirulent in an avian(s) by a method(s) described herein or known to one of skill in the art. In a specific embodiment, the APMV-2, APMV-3, APMV-4, APMV-6, APMV-7, APMV-8, or APMV-9 strain that is used in a method of treating cancer described herein is recombinantly produced. In certain embodiments, the APMV-2, APMV-3, APMV-4, APMV-6, APMV-7, APMV-8, or APMV-9 strain that is used in a method of treating cancer described herein is genetically engineered to be attenuated in a manner that attenuates the pathogenicity of the virus in birds.

In another specific embodiment, the APMV-2, APMV-3, APMV-4, APMV-6, APMV-7, APMV-8, or APMV-9 strain that is used in a method of treating cancer described herein has an intracerebral pathogenicity index in day-old chicks of the Gallus gallus species of less than 0.7. In certain specific embodiments, the APMV-2, APMV-3, APMV-4, APMV-6, APMV-7, APMV-8, or APMV-9 strain that is used in a method of treating cancer described herein is not pathogenic as assessed by intracranial injection of 1-day-old chicks with the virus, and disease development and death as scored for 8 days. In some embodiments, the APMV-2, APMV-3, APMV-4, APMV-6, APMV-7, APMV-8, or APMV-9 strain that is used in a method of treating cancer described herein has an intracranial pathogenicity index of less than 0.7, less than 0.6, less than 0.5, less than 0.4, less than 0.3, less than 0.2 or less than 0.1. In some embodiments, the APMV-2, APMV-3, APMV-4, APMV-6, APMV-7, APMV-8, or APMV-9 strain that is used in a method of treating cancer described herein has an intracranial pathogenicity index between 0.7 to 0.1, 0.6 to 0.1, 0.5 to 0.1 or 0.4 to 0.1. In certain embodiments, the APMV-2, APMV-3, APMV-4, APMV-6, APMV-7, APMV-8, or APMV-9 strain that is used in a method of treating cancer described herein has an intracranial pathogenicity index of zero. See, e.g,. one or more of the following references for a description of an assay that may be used to assess the pathogenicity of an APMV in birds: Hines, N. L. and C. L. Miller, Avian paramyxovirus serotype-1: a review of disease distribution, clinical symptoms, and laboratory diagnostics. Vet Med Int, 2012. 2012: p. 708216; Kim S-H, Xiao S, Shive H, Collins PL, Samal S K., 2012: Replication, Neurotropism, and Pathogenicity of Avian Paramyxovirus Serotypes 1-9 in Chickens and Ducks. PLoS ONE. ;7(4): e34927; Subbiah, M., Xiao, S., Khattar, S. K., Dias, F. M., Collins, P. L., & Samal, S. K., 2010: Pathogenesis of two strains of Avian Paramyxovirus serotype 2, Yucaipa and Bangor, in chickens and turkeys. Avian Diseases, 54(3), 1050-1057; Kumar S, Militino Dias F, Nayak B, Collins PL, Samal S. K., 2010: Experimental avian paramyxovirus serotype-3 infection in chickens and turkeys. Veterinary Research.; 41(5):72; Ryota Tsunekuni, Hirokazu Hikono, Takehiko Saito., 2014: Evaluation of avian paramyxovirus serotypes 2 to 10 as vaccine vectors in chickens previously immunized against Newcastle disease virus. Veterinary Immunology and Immunopathology; 160(3-4):184-191; and www.oie.int/fileadmin/Home/fr/Health_standards/tahm/2.03.14_NEWCASTLE DIS.pdf, each of which is incorporated herein by reference in its entirety. In a specific embodiment, the APMV-2, APMV-3, APMV-4, APMV-6, APMV-7, APMV-8, or APMV-9 strain is a recombinant APMV-2, APMV-3, APMV-4, APMV-6, APMV-7, APMV-8, or APMV-9 strain, respectively.

In another specific embodiment, the APMV-2, APMV-3, APMV-4, APMV-6, APMV-7, APMV-8, or APMV-9 strain that is used in a method of treating cancer described herein is naturally occurring and has an intracerebral pathogenicity index in day-old chicks of the Gallus gallus species of less than 0.7. In a specific embodiment, the APMV-2, APMV-3, APMV-4, APMV-6, APMV-7, APMV-8, or APMV-9 strain that is used in a method of treating cancer described herein is a recombinant APMV-2, APMV-3, APMV-4, APMV-6, APMV-7, APMV-8, or APMV-9 strain, respectively, and has an intracerebral pathogenicity index in day-old chicks of the Gallus gallus species of less than 0.7.

In a specific embodiment, an APMV-2, APMV-3, APMV-4, APMV-6, APMV-7, APMV-8, or APMV-9 that is used in a method of treating cancer described herein decreases tumor growth and increases survival in a B16-F10 syngeneic murine melanoma model as compared to tumor growth and survival in B16-F10 syngeneic murine melanoma model administered phosphate buffered saline (PBS). In another specific embodiments, an APMV-2, APMV-3, APMV-4, APMV-6, APMV-7, APMV-8, or APMV-9 that is used in a method of treating cancer described herein results in a greater decrease in tumor growth and a longer survival time in a B16-F10 syngeneic murine melanoma model as compared to tumor growth and survival time in the B16-F10 syngeneic murine melanoma model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A (for a description of the L289A mutation, see, e.g., Sergel et al. (2000) A Single Amino Acid Change in the Newcastle Disease Virus Fusion Protein Alters the Requirement for HN Protein in Fusion. Journal of Virology 74(11): 5101-5107, which is incorporated herein by reference in its entirety). In another specific embodiments, an APMV-2, APMV-3, APMV-4, APMV-6, APMV-7, APMV-8, or APMV-9 that is used in a method of treating cancer described herein results in a comparable decrease in tumor growth and increase survival time in a B16-F10 syngeneic murine melanoma model as compared to tumor growth and survival time in the B16-F10 syngeneic murine melanoma model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, the modified NDV comprises a packaged genome, wherein the packaged genome comprises the negative sense RNA transcribed from the cDNA sequence set forth in SEQ ID NO:13.

In a specific embodiment, an APMV-2, APMV-3, APMV-4, APMV-6, APMV-7, APMV-8, or APMV-9 that is used in a method of treating cancer described herein decreases tumor growth and increases survival in a BALBc syngeneic murine colon carcinoma tumor model as compared to tumor growth and survival in BALBc syngeneic murine colon carcinoma tumor model administered phosphate buffered saline (PBS). In a specific embodiment, an APMV-2, APMV-3, APMV-4, APMV-6, APMV-7, APMV-8, or APMV-9 that is used in a method of treating cancer described herein results in a greater decrease in tumor growth and a longer survival time in a BALBc syngeneic murine colon carcinoma tumor model as compared to tumor growth and survival time in the BALBc syngeneic murine colon carcinoma tumor model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, an APMV-2, APMV-3, APMV-4, APMV-6, APMV-7, APMV-8, or APMV-9 that is used in a method of treating cancer described herein results in a comparable decrease in tumor growth and increase survival time in a BALBc syngeneic murine colon carcinoma tumor model as compared to tumor growth and survival time in the BALBc syngeneic murine colon carcinoma tumor model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, the modified NDV comprises a packaged genome, wherein the packaged genome comprises the negative sense RNA transcribed from the cDNA sequence set forth in SEQ ID NO:13.

In a specific embodiment, an APMV-2, APMV-3, APMV-4, APMV-6, APMV-7, APMV-8, or APMV-9 that is used in a method of treating cancer described herein decreases tumor growth and increases survival in a C57BL/6 syngeneic murine lung carcinoma tumor model as compared to tumor growth and survival in C57BL/6 syngeneic murine lung carcinoma tumor model administered phosphate buffered saline (PBS). In a specific embodiment, an APMV-2, APMV-3, APMV-4, APMV-6, APMV-7, APMV-8, or APMV-9 that is used in a method of treating cancer described herein results in a greater decrease in tumor growth and a longer survival time in a C57BL/6 syngeneic murine lung carcinoma tumor model as compared to tumor growth and survival time in the C57BL/6 syngeneic murine lung carcinoma tumor model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, an APMV-2, APMV-3, APMV-4, APMV-6, APMV-7, APMV-8, or APMV-9 that is used in a method of treating cancer described herein results in a comparable decrease in tumor growth and increase survival time in a C57BL/6 syngeneic murine lung carcinoma tumor model as compared to tumor growth and survival time in the C57BL/6 syngeneic murine lung carcinoma tumor model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, the modified NDV comprises a packaged genome, wherein the packaged genome comprises the negative sense RNA transcribed from the cDNA sequence set forth in SEQ ID NO: 13.

In a specific embodiment, an APMV strain is used in a method for treating cancer described herein is an APMV-2, APMV-3, APMV-4, APMV-6, APMV-7, APMV-8, or APMV-9 described in Section 6, infra. In one embodiment, an APMV-2 strain is used in a method for treating cancer described herein, wherein the APMV-2 strain is APMV-2 Chicken/California/Yucaipa/1956. See, e.g., GenBank No. EU338414.1 or SEQ ID NO:1 for the complete genomic cDNA sequence of APMV-2 Chicken/California/Yucaipa/1956. In another embodiment, an APMV-3 strain is used in a method for treating cancer described herein, wherein the APMV-3 strain is APMV-3 turkey/Wisconsin/68. See, e.g., GenBank No. EU782025.1 or SEQ ID NO:2 for the complete genomic cDNA sequence of APMV-3 turkey/Wisconsin/68. In another embodiment, an APMV-6 strain is used in a method for treating cancer described herein, wherein the APMV-6 strain is APMV-6/duck/Hong Kong/18/199/77. See, e.g., GenBank No. EU622637.2 or SEQ ID NO:9 for the complete genomic cDNA sequence of APMV-6/duck/Hong Kong/18/199/77. In another embodiment, an APMV-7 strain is used in a method for treating cancer described herein, wherein the APMV-7 strain is APMV-7/dove/Tennessee/4/75. See, e.g., GenBank No. FJ231524.1 or SEQ ID NO:10 for the complete genomic cDNA of APMV-7/dove/Tennessee/4/75. In another embodiment, an APMV-8 strain is used in a method for treating cancer described herein, wherein the APMV-8 strain is APMV-8/Goose/Delaware/1053/76. See, e.g., GenBank No. FJ619036.1 or SEQ ID NO:11 for the complete genomic cDNA sequence of APMV-8/Goose/Delaware/1053/76. In another embodiment, an APMV-9 is used in a method for treating cancer described herein, wherein the APMV-9 strain is APMV-9 duck/New York/22/1978. See, e.g., GenBank No. NC_025390.1 or SEQ ID NO:12 for the complete genomic cDNA sequence of APMV-9 duck/New York/22/1978.

In a specific embodiment, an APMV-4 strain is used in a method for treating cancer described herein. In another embodiment, an APMV-4 strain that is naturally occurring is used in a method of treating cancer described herein. In a preferred embodiment, an APMV-4 strain that is naturally occurring and has an intracerebral pathogenicity index in day-old chicks of the Gallus gallus species of less than 0.7 is used in a method of treating cancer described herein. In a preferred embodiment, the APMV-4 that is used in a method of treating cancer described herein is APMV-4/Duck/Hong Kong/D3/1975 strain. See, e.g., GenBank No. FJ177514.1 or SEQ ID NO:4 for the complete genomic cDNA sequence of APMV-4/duck/Hong Kong/D3/75. In a specific embodiment, the APMV-4 that is used in a method of treating cancer described herein is APMV-4/Duck/China/G302/2012 strain, APMV4/mallard/Belgium/15129/07 strain, APMV4/Uriah_aalge/Russia/Tyuleniy_Island/115/2015 strain, APMV-4/Egyptian goose/South Africa/N1468/2010 strain, or APMV4/duck/Delaware/549227/2010 strain. In a specific embodiment, the APMV-4 that is used in a method of treating cancer described herein is an APMV-4 with a genome that has 80%, 85%, 90%, 95% or higher percent identity to the genome of APMV-4/Duck/Hong Kong/D3/1975 strain.

In one embodiment, the APMV-4 that is used in a method of treating cancer described herein is APMV-4/Duck/China/G302/2012 strain. See, e.g., GenBank No. KC439346.1 or SEQ ID NO:7 for the complete genomic cDNA sequence of APMV-4/Duck/China/G302/2012 strain. In another embodiment, the APMV-4 that is used in a method of treating cancer described herein is APMV-4/Uriah_aalge/Russia/Tyuleniy_Island/115/2015 strain. See, e.g., GenBank No. KU601399.1 or SEQ ID NO:5 for the complete genomic cDNA sequence of APMV-4/Uriah_aalge/Russia/Tyuleniy_Island/115/2015 strain. In another embodiment, the APMV-4 that is used in a method of treating cancer described herein is APMV4/duck/Delaware/549227/2010 strain. See, e.g., GenBank No. JX987283.1 or SEQ ID NO:8 for the complete genomic cDNA sequence of APMV4/duck/Delaware/549227/2010 strain. In another embodiment, the APMV-4 that is used in a method of treating cancer described herein is APMV4/mallard/Belgium/15129/07 strain. See, e.g., GenBank No. JN571485 or SEQ ID NO:3 for the complete genomic cDNA sequence of APMV4/mallard/Belgium/15129/07 strain. In another embodiment, the APMV-4 that is used in a method of treating cancer described herein is APMV-4/Egyptian goose/South Africa/N1468/2010 strain. See, e.g., GenBank No. JX133079.1 or SEQ ID NO:6 for the complete genomic cDNA sequence of APMV-4/Egyptian goose/South Africa/N1468/2010 strain.

In a specific embodiment, an APMV-4 that is used in a method of treating cancer described herein decreases tumor growth and increases survival in a B16-F10 syngeneic murine melanoma model as compared to tumor growth and survival in B16-F10 syngeneic murine melanoma model administered phosphate buffered saline (PBS). In another specific embodiment, an APMV-4 that is used in a method of treating cancer described herein results in a greater decrease in tumor growth and a longer survival time in a B16-F10 syngeneic murine melanoma model as compared to tumor growth and survival time in the B16-F10 syngeneic murine melanoma model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, the modified NDV comprises a packaged genome, wherein the packaged genome comprises the negative sense RNA transcribed from the cDNA sequence set forth in SEQ ID NO:13.

In a specific embodiment, an APMV-4 that is used in a method of treating cancer described herein decreases tumor growth and increases survival in a BALBc syngeneic murine colon carcinoma tumor model as compared to tumor growth and survival in BALBc syngeneic murine colon carcinoma tumor model administered phosphate buffered saline (PBS). In a specific embodiment, an APMV-4 that is used in a method of treating cancer described herein results in a greater decrease in tumor growth and a longer survival time in a BALBc syngeneic murine colon carcinoma tumor model as compared to tumor growth and survival time in the BALBc syngeneic murine colon carcinoma tumor model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, the modified NDV comprises a packaged genome, wherein the packaged genome comprises the negative sense RNA transcribed from the cDNA sequence set forth in SEQ ID NO: 13.

In a specific embodiment, an APMV-4 that is used in a method of treating cancer described herein decreases tumor growth and increases survival in a C57BL/6 syngeneic murine lung carcinoma tumor model as compared to tumor growth and survival in C57BL/6 syngeneic murine lung carcinoma tumor model administered phosphate buffered saline (PBS). In a specific embodiment, an APMV-4 that is used in a method of treating cancer described herein results in a greater decrease in tumor growth and a longer survival time in a C57BL/6 syngeneic murine lung carcinoma tumor model as compared to tumor growth and survival time in the C57BL/6 syngeneic murine lung carcinoma tumor model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, the modified NDV comprises a packaged genome, wherein the packaged genome comprises the negative sense RNA transcribed from the cDNA sequence set forth in SEQ ID NO: 13.

In a specific embodiment, an APMV-8 strain is used in a method for treating cancer described herein. In another embodiment, an APMV-8 strain that is naturally occurring is used in a method of treating cancer described herein. In a specific embodiment, an APMV-8 strain that is naturally occurring and has an intracerebral pathogenicity index in day-old chicks of the Gallus gallus species of less than 0.7 is used in a method of treating cancer described herein. In a specific embodiment, the APMV-8 that is used in a method of treating cancer described herein is APMV-8/Goose/Delaware/1053/76. See, e.g., GenBank No. FJ619036.1 or SEQ ID NO:11 for the complete genomic cDNA sequence of APMV-8/Goose/Delaware/1053/76. In a specific embodiment, the APMV-8 that is used in a method of treating cancer described herein is an APMV-8 with a genome that has 80%, 85%, 90%, 95% or higher percent identity to the genome of APMV-8/Goose/Delaware/1053/76.

In a specific embodiment, an APMV-7 strain is used in a method for treating cancer described herein. In another embodiment, an APMV-7 strain that is naturally occurring is used in a method of treating cancer described herein. In a preferred embodiment, an APMV-7 strain that is naturally occurring and has an intracerebral pathogenicity index in day-old chicks of the Gallus gallus species of less than 0.7 is used in a method of treating cancer described herein. In a specific embodiment, the APMV-7 that is used in a method of treating cancer described herein is APMV-7/dove/Tennessee/4/75. See, e.g., GenBank No. FJ231524.1 or SEQ ID NO:10 for the complete genomic cDNA of APMV-7/dove/Tennessee/4/75. In a specific embodiment, the APMV-7 that is used in a method of treating cancer described herein is and APMV-7 with a genome that has 80%, 85%, 90%, 95% or higher percent identity to the genome of APMV-7/dove/Tennessee/4/75.

In a specific embodiment, an APMV-2 strain is used in a method for treating cancer described herein. In another embodiment, an APMV-2 strain that is naturally occurring is used in a method of treating cancer described herein. In a preferred embodiment, an APMV-2 strain that is naturally occurring and has an intracerebral pathogenicity index in day-old chicks of the Gallus gallus species of less than 0.7 is used in a method of treating cancer described herein. In a specific embodiment, the APMV-2 that is used in a method of treating cancer described herein is APMV-2 Chicken/California/Yucaipa/1956. See, e.g., GenBank No. EU338414.1 or SEQ ID NO:1 for the complete genomic cDNA sequence of APMV-2 Chicken/California/Yucaipa/1956. In a specific embodiment, the APMV-2 that is used in a method of treating cancer described herein is and APMV-2 with a genome that has 80%, 85%, 90%, 95% or higher percent identity to the genome of APMV-2 Chicken/California/Yucaipa/1956.

In a specific embodiment, an APMV-3 strain is used in a method for treating cancer described herein. In another embodiment, an APMV-3 strain that is naturally occurring is used in a method of treating cancer described herein. In a preferred embodiment, an APMV-3 strain that is naturally occurring and has an intracerebral pathogenicity index in day-old chicks of the Gallus gallus species of less than 0.7 is used in a method of treating cancer described herein. In a specific embodiment, the APMV-3 that is used in a method of treating cancer described herein is APMV-3 turkey/Wisconsin/68. See, e.g., GenBank No. EU782025.1 or SEQ ID NO:2 for the complete genomic cDNA sequence of APMV-3 turkey/Wisconsin/68. In a specific embodiment, the APMV-3 that is used in a method of treating cancer described herein is and APMV-3 with a genome that has 80%, 85%, 90%, 95% or higher percent identity to the genome of APMV-3 turkey/Wisconsin/68.

In a specific embodiment, an APMV-6 strain is used in a method for treating cancer described herein. In another embodiment, an APMV-6 strain that is naturally occurring is used in a method of treating cancer described herein. In a preferred embodiment, an APMV-6 strain that is naturally occurring and has an intracerebral pathogenicity index in day-old chicks of the Gallus gallus species of less than 0.7 is used in a method of treating cancer described herein. In a specific embodiment, the APMV-6 that is used in a method of treating cancer described herein is APMV-6/duck/Hong Kong/18/199/77. See, e.g., GenBank No. EU622637.2 or SEQ ID NO:9 for the complete genomic cDNA sequence of APMV-6/duck/Hong Kong/18/199/77. In a specific embodiment, the APMV-6 that is used in a method of treating cancer described herein is an APMV-6 with a genome that has 80%, 85%, 90%, 95% or higher percent identity to the genome of APMV-6/duck/Hong Kong/18/199/77.

In a specific embodiment, an APMV-9 strain is used in a method for treating cancer described herein. In another embodiment, an APMV-9 strain that is naturally occurring is used in a method of treating cancer described herein. In a preferred embodiment, an APMV-9 strain that is naturally occurring and has an intracerebral pathogenicity index in day-old chicks of the Gallus gallus species of less than 0.7 is used in a method of treating cancer described herein. In a specific embodiment, the APMV-9 that is used in a method of treating cancer described herein is APMV-9 duck/New York/22/1978. See, e.g., GenBank No. NC_025390.1 or SEQ ID NO:12 for the complete genomic cDNA sequence of APMV-9 duck/New York/22/1978. In a specific embodiment, the APMV-9 that is used in a method of treating cancer described herein is an APMV-9 with a genome that has 80%, 85%, 90%, 95% or higher percent identity to the genome of APMV-9 duck/New York/22/1978.

### 5.1.2 Recombinant APMV

In one aspect, presented herein are recombinant APMVs comprising a packaged genome, wherein the packaged genome comprises a transgene. See, e.g., Section 5.1.2.2 and Section 7 for examples of transgenes which may be incorporated into the genome of an APMV described herein. See, e.g., Section 5.1.2.1 and Section 6 for examples of APMVs, the genome of which a transgene may be incorporated. In a particular embodiment, the genome of the APMV, which the transgene is incorporated, is the genome of an APMV-4 (e.g., an APMV-4 strain described herein), APMV-7 strain (e.g., an APMV-7 strain described herein) or APMV-8 strain (e.g., an APMV-8 strain described herein). In another embodiment, the genome of the APMV in which the transgene is incorporated is the genome of an APMV-6 (e.g., an APMV-6 strain described herein) or APMV-9 strain (e.g., an APMV-9 strain described herein). In a specific embodiment, provided herein is a recombinant APMV-4 comprising a packaged genome, wherein the packaged genome comprises a transgene. In a preferred embodiment, provided herein is a recombinant APMV-4 comprising a packaged genome, wherein the packaged genome comprises (consists of) the negative sense RNA transcribed from the cDNA sequence set forth in SEQ ID NO:14. In a specific embodiment, the protein encoded by the transgene is expressed by cells infected with the recombinant APMV.

In certain embodiments, the genome of the recombinant APMV does not comprise a heterologous sequence encoding a heterologous protein other than the protein encoded by the transgene. In certain embodiments, a recombinant APMV described herein comprises a packaged genome, wherein the genome comprises (or consists of) the genes found in APMV and a transgene. In certain embodiments, a recombinant APMV described herein comprises a packaged genome, wherein the genome comprises (or consists of) the transcription units found in APMV (e.g., transcription units for APMV nucleocapsid, protein, phosphoprotein, matrix protein, fusion protein, hemagglutinin-neuraminidase protein, and large polymerase protein) and a transgene (e.g., in Section 5.1.2.2), but does not include another other transgenes.

### 5.1.2.1 Backbone of the Recombinant APMV

Any APMV-2, APMV-3, APMV-4, APMV-6, APMV-7, APMV-8, or APMV-9 strain may serve as the “backbone” that is engineered to encode a transgene described herein, including, but not limited to, naturally-occurring strains, variants or mutants, mutagenized viruses, or genetically engineered viruses, or any combination thereof In certain embodiments, the APMV-2, APMV-3, APMV-4, APMV-6, APMV-7, APMV-8, or APMV-9 strain that is engineered to encode a transgene described herein is a lytic strain. In other embodiments, the APMV-2, APMV-3, APMV-4, APMV-6, APMV-7, APMV-8, or APMV-9 strain that is engineered to encode a transgene described herein is a non-lytic strain. In a specific embodiment, a transgene described herein is incorporated into the genome of APMV-2, APMV-3, APMV-4, APMV-6, APMV-7, APMV-8, or APMV-9 strain that is avirulent in an avian(s) by a method(s) described herein or known to one of skill in the art. In certain embodiments, the APMV-2, APMV-3, APMV-4, APMV-6, APMV-7, APMV-8, or APMV-9 strain that is engineered to encode a transgene described herein is genetically engineered to be attenuated in a manner that attenuates the pathogenicity of the virus in birds.

In another specific embodiment, a transgene is incorporated into the genome of an APMV-2, APMV-3, APMV-4, APMV-6, APMV-7, APMV-8, or APMV-9 strain that has an intracerebral pathogenicity index in day-old chicks of the Gallus gallus species of less than 0.7. In certain specific embodiments, the APMV-2, APMV-3, APMV-4, APMV-6, APMV-7, APMV-8, or APMV-9 strain that is engineered to encode a transgene described herein is not pathogenic as assessed by intracranial injection of 1-day-old chicks with the virus, and disease development and death as scored for 8 days. In some embodiments, the APMV-2, APMV-3, APMV-4, APMV-6, APMV-7, APMV-8, or APMV-9 strain that is engineered to encode a transgene described herein has an intracranial pathogenicity index of less than 0.7, less than 0.6, less than 0.5, less than 0.4, less than 0.3, less than 0.2 or less than 0.1. In some embodiments, the APMV-2, APMV-3, APMV-4, APMV-6, APMV-7, APMV-8, or APMV-9 strain that is engineered to encode a transgene described herein has an intracranial pathogenicity index between 0.7 to 0.1, 0.6 to 0.1, 0.5 to 0.1 or 0.4 to 0.1. In certain embodiments, the APMV-2, APMV-3, APMV-4, APMV-6, APMV-7, APMV-8, or APMV-9 strain that is engineered to encode a transgene described herein has an intracranial pathogenicity index of zero. See, e.g,. one or more of the following references for a description of an assay that may be used to assess the pathogenicity of an APMV in birds: Hines, N. L. and C. L. Miller, Avian paramyxovirus serotype-1: a review of disease distribution, clinical symptoms, and laboratory diagnostics. Vet Med Int, 2012. 2012: p. 708216; Kim S-H, Xiao S, Shive H, Collins P L, Samal S K., 2012: Replication, Neurotropism, and Pathogenicity of Avian Paramyxovirus Serotypes 1-9 in Chickens and Ducks. PLoS ONE.; 7(4): e34927; Subbiah, M., Xiao, S., Khattar, S. K., Dias, F. M., Collins, P. L., & Samal, S. K., 2010: Pathogenesis of two strains of Avian Paramyxovirus serotype 2, Yucaipa and Bangor, in chickens and turkeys. Avian Diseases, 54(3), 1050-1057; Kumar S, Militino Dias F, Nayak B, Collins P L, Samal S. K., 2010: Experimental avian paramyxovirus serotype-3 infection in chickens and turkeys. Veterinary Research.; 41(5):72; Ryota Tsunekuni, Hirokazu Hikono, Takehiko Saito.,2014: Evaluation of avian paramyxovirus serotypes 2 to 10 as vaccine vectors in chickens previously immunized against Newcastle disease virus. Veterinary Immunology and Immunopathology; 160(3-4):184-191; and www.oie.int/fileadmin/Home/fr/Health_standards/tahm/2.03.14 NEWCASTLE DIS.pdf, each of which is incorporated herein by reference in its entirety.

In a specific embodiment, a transgene described herein is incorporated into the genome of an APMV-2, APMV-3, APMV-4, APMV-6, APMV-7, APMV-8, or APMV-9 that decreases tumor growth and increases survival in a B16-F10 syngeneic murine melanoma model as compared to tumor growth and survival in B16-F10 syngeneic murine melanoma model administered phosphate buffered saline (PBS). In another specific embodiment, a transgene described herein is incorporated into the genome of an APMV-2, APMV-3, APMV-4, APMV-6, APMV-7, APMV-8, or APMV-9 that results in a greater decrease in tumor growth and a longer survival time in a B16-F10 syngeneic murine melanoma model as compared to tumor growth and survival time in the B16-F10 syngeneic murine melanoma model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In another specific embodiment, a transgene described herein is incorporated into the genome of an APMV-2, APMV-3, APMV-4, APMV-6, APMV-7, APMV-8, or APMV-9 that results in a comparable decrease in tumor growth and increase survival time in a B16-F10 syngeneic murine melanoma model as compared to tumor growth and survival time in the B16-F10 syngeneic murine melanoma model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, the modified NDV comprises a packaged genome, wherein the packaged genome comprises the negative sense RNA transcribed from the cDNA sequence set forth in SEQ ID NO:13.

In a specific embodiment, a transgene described herein is incorporated into the genome of an APMV-2, APMV-3, APMV-4, APMV-6, APMV-7, APMV-8, or APMV-9 that decreases tumor growth and increases survival in a BALBc syngeneic murine colon carcinoma tumor model as compared to tumor growth and survival in BALBc syngeneic murine colon carcinoma tumor model administered phosphate buffered saline (PBS). In a specific embodiment, a transgene described herein is incorporated into the genome of an APMV-2, APMV-3, APMV-4, APMV-6, APMV-7, APMV-8, or APMV-9 that results in a greater decrease in tumor growth and a longer survival time in a BALBc syngeneic murine colon carcinoma tumor model as compared to tumor growth and survival time in the BALBc syngeneic murine colon carcinoma tumor model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, a transgene described herein is incorporated into the genome of an APMV-2, APMV-3, APMV-4, APMV-6, APMV-7, APMV-8, or APMV-9 that results in a comparable decrease in tumor growth and increase survival time in a BALBc syngeneic murine colon carcinoma tumor model as compared to tumor growth and survival time in the BALBc syngeneic murine colon carcinoma tumor model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, the modified NDV comprises a packaged genome, wherein the packaged genome comprises the negative sense RNA transcribed from the cDNA sequence set forth in SEQ ID NO:13.

In a specific embodiment, a transgene described herein is incorporated into the genome of an APMV-2, APMV-3, APMV-4, APMV-6, APMV-7, APMV-8, or APMV-9 that decreases tumor growth and increases survival in a C57BL/6 syngeneic murine lung carcinoma tumor model as compared to tumor growth and survival in C57BL/6 syngeneic murine lung carcinoma tumor model administered phosphate buffered saline (PBS). In a specific embodiment, a transgene described herein is incorporated into the genome of an APMV-2, APMV-3, APMV-4, APMV-6, APMV-7, APMV-8, or APMV-9 that results in a greater decrease in tumor growth and a longer survival time in a C57BL/6 syngeneic murine lung carcinoma tumor model as compared to tumor growth and survival time in the C57BL/6 syngeneic murine lung carcinoma tumor model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, a transgene described herein is incorporated into the genome of an APMV-2, APMV-3, APMV-4, APMV-6, APMV-7, APMV-8, or APMV-9 that results in a comparable decrease in tumor growth and increase survival time in a C57BL/6 syngeneic murine lung carcinoma tumor model as compared to tumor growth and survival time in the C57BL/6 syngeneic murine lung carcinoma tumor model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, the modified NDV comprises a packaged genome, wherein the packaged genome comprises the negative sense RNA transcribed from the cDNA sequence set forth in SEQ ID NO:13.

In a specific embodiment, a transgene described herein is incorporated into the genome of an APMV-4 strain. In a preferred embodiment, a transgene described herein is incorporated into the genome of APMV-4/Duck/Hong Kong/D3/1975 strain. One example of a cDNA sequence of the genome of the APMV-4/Duck/Hong Kong/D3/1975 strain may be found in SEQ ID NO:4. In a specific embodiment, the nucleotide sequence of a transgene described herein is incorporated into the genome of APMV-4/Duck/China/G302/2012 strain, APMV4/mallard/Belgium/15129/07 strain, APMV4/Uriah_aalge/Russia/Tyuleniy_Island/115/2015 strain, APMV4/Egyptian goose/South Africa/N1468/2010 strain, or APMV-4/duck/Delaware/549227/2010 strain. One example of a cDNA sequence of the genome of the APMV-4/Duck/China/G302/2012 strain may be found in SEQ ID NO:7. An example of a cDNA sequence of the genome of the APMV4/mallard/Belgium/15129/07 strain may be found in SEQ ID NO:3. An example of a cDNA sequence of the genome of the APMV4/Uriah_aalge/Russia/Tyuleniy_Island/115/2015 strain may be found in SEQ ID NO:5. An example of a cDNA sequence of the genome of the APMV4/Egyptian goose/South Africa/N1468/2010 strain may be found in SEQ ID NO:6. An example of a cDNA sequence of the genome of the APMV-4/duck/Delaware/549227/2010 strain may be found in SEQ ID NO:8.

In a specific embodiment, a transgene described herein is incorporated into the genome of an APMV-4 that decreases tumor growth and increases survival in a B16-F10 syngeneic murine melanoma model as compared to tumor growth and survival in B16-F10 syngeneic murine melanoma model administered phosphate buffered saline (PBS). In another specific embodiments, a transgene described herein is incorporated into the genome of an APMV-4 that results in a greater decrease in tumor growth and a longer survival time in a B16-F10 syngeneic murine melanoma model as compared to tumor growth and survival time in the B16-F10 syngeneic murine melanoma model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, the modified NDV comprises a packaged genome, wherein the packaged genome comprises the negative sense RNA transcribed from the cDNA sequence set forth in SEQ ID NO:13.

In a specific embodiment, a transgene described herein is incorporated into the genome of an APMV-4 that decreases tumor growth and increases survival in a BALBc syngeneic murine colon carcinoma tumor model as compared to tumor growth and survival in BALBc syngeneic murine colon carcinoma tumor model administered phosphate buffered saline (PBS). In a specific embodiment, a transgene described herein is incorporated into the genome of an APMV-4 that results in a greater decrease in tumor growth and a longer survival time in a BALBc syngeneic murine colon carcinoma tumor model as compared to tumor growth and survival time in the BALBc syngeneic murine colon carcinoma tumor model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, the modified NDV comprises a packaged genome, wherein the packaged genome comprises the negative sense RNA transcribed from the cDNA sequence set forth in SEQ ID NO:13.

In a specific embodiment, a transgene described herein is incorporated into the genome of an APMV-4 that decreases tumor growth and increases survival in a C57BL/6 syngeneic murine lung carcinoma tumor model as compared to tumor growth and survival in C57BL/6 syngeneic murine lung carcinoma tumor model administered phosphate buffered saline (PBS). In a specific embodiment, a transgene described herein is incorporated into the genome of an APMV-4 that results in a greater decrease in tumor growth and a longer survival time in a C57BL/6 syngeneic murine lung carcinoma tumor model as compared to tumor growth and survival time in the C57BL/6 syngeneic murine lung carcinoma tumor model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A. In a specific embodiment, the modified NDV comprises a packaged genome, wherein the packaged genome comprises the negative sense RNA transcribed from the cDNA sequence set forth in SEQ ID NO:13.

In a specific embodiment, a transgene described herein is incorporated into the genome of an APMV-7 strain. In a particular embodiment, a transgene described herein is incorporated into the genome of is APMV-7/dove/Tennessee/4/75. See, e.g., GenBank No. FJ231524.1 or SEQ ID NO:10 for the complete genomic cDNA of APMV-7/dove/Tennessee/4/75.

In a specific embodiment, a transgene described herein is incorporated into the genome of an APMV-8 strain. In a particular embodiment, a transgene described herein is incorporated into the genome of APMV-8/Goose/Delaware/1053/76. See, e.g., GenBank No. FJ619036.1 or SEQ ID NO:11 for the complete genomic cDNA sequence of APMV-8/Goose/Delaware/1053/76.

In a specific embodiment, a transgene described herein is incorporated into the genome of an APMV-9 strain. In a particular embodiment, a transgene described herein is incorporated into the genome of APMV-9 duck/New York/22/1978. See, e.g., GenBank No. NC_025390.1 or SEQ ID NO:12 for the complete genomic cDNA sequence of APMV-9 duck/New York/22/1978.

In a specific embodiment, a transgene described herein is incorporated into the genome of an APMV-2 strain. In a particular embodiment, a transgene described herein is incorporated into the genome of APMV-2 Chicken/California/Yucaipa/1956. See, e.g., GenBank No. EU338414.1 or SEQ ID NO:1 for the complete genomic cDNA sequence of APMV-2 Chicken/California/Yucaipa/1956.

In a specific embodiment, a transgene described herein is incorporated into the genome of an APMV-3 strain. In a particular embodiment, a transgene described herein is incorporated into the genome of APMV-3 turkey/Wisconsin/68. See, e.g., GenBank No. EU782025.1 or SEQ ID NO:2 for the complete genomic cDNA sequence of APMV-3 turkey/Wisconsin/68.

In a specific embodiment, a transgene described herein is incorporated into the genome of an APMV-6 strain. In a particular embodiment, a transgene described herein is incorporated into the genome of APMV-6/duck/Hong Kong/18/199/77. See, e.g., GenBank No. EU622637.2 or SEQ ID NO:9 for the complete genomic cDNA sequence of APMV-6/duck/Hong Kong/18/199/77.

One skilled in the art will understand that the APMV genomic RNA sequence is the reverse complement of a cDNA sequence encoding the APMV genome. Thus, any program that generates converts a nucleotide sequence to its reverse complement sequence may be utilized to convert a cDNA sequence encoding an APMV genome into the genomic RNA sequence (see, e.g., www.bioinformatics.org/sms/rev_comp.html, www.fr33.net/seqedit.php, and DNAStar). Accordingly, the nucleotide sequences provided in Tables 2 and 3, infra, may be readily converted to the negative-sense RNA sequence of the APMV genome by one of skill in the art.

In a specific embodiment, a transgene is incorporated into the genome of an APMV-4 strain, wherein the genome comprises the transcription units of the APMV-4 strain necessary for infection and replication of the virus in a substrate (e.g., a cell line susceptible to APMV-4 infection), subject (e.g., a human subject), or both. In a specific embodiment, a transgene is incorporated into the genome of an APMV-4 strain, wherein the genome comprises a transcription unit encoding the APMV-4 nucleocapsid (N) protein, a transcription unit encoding the APMV-4 phosphoprotein (P), a transcription unit encoding the APMV-4 matrix (M) protein, a transcription unit encoding the APMV-4 fusion (F) protein, a transcription unit encoding the APMV-4 hemagglutinin-neuraminidase (HN) protein, and a transcription unit encoding the APMV-4 large polymerase (L) protein. The transgene may be incorporated into the APMV-4 genome between two transcription units of an APMV-4 described herein (e.g., between the M and P transcription units or between the HN and L transcription units). In certain embodiments, the genome of the APMV-4 does not encode a heterologous protein other than a transgene described herein. In a specific embodiment, the APMV-4 strain is the APMV-4/Duck/Hong Kong/D3/1975 strain, APMV-4/Duck/China/G302/2012 strain, APMV4/mallard/Belgium/15129/07 strain, APMV4Uriah-aalge/Russia/Tyuleniy_Island/115/2015 strain, APMV4/Egyptian goose/South Africa/NJ468/2010 strain, or APMV4/duck/Delaware/549227/2010 strain.

In a specific embodiment, a transgene is incorporated into the genome of an APMV-8 strain, wherein the genome comprises the transcription units of the APMV-8 strain necessary for infection and replication of the virus in a substrate (e.g., a cell line susceptible to APMV-8 infection), subject (e.g., a human subject), or both. In a specific embodiment, a transgene is incorporated into the genome of an APMV-8 strain, wherein the genome comprises a transcription unit encoding the APMV-8 nucleocapsid (N) protein, a transcription unit encoding the APMV-8 phosphoprotein (P), a transcription unit encoding the APMV-8 matrix (M) protein, a transcription unit encoding the APMV-8 fusion (F) protein, a transcription unit encoding the APMV-8 hemagglutinin-neuraminidase (HN) protein, and a transcription unit encoding the APMV-8 large polymerase (L) protein. The transgene may be incorporated into the APMV-8 genome between two transcription units of an APMV-8 described herein (e.g., between the M and P transcription units or between the HN and L transcription units). In certain embodiments, the genome of the APMV-8 does not encode a heterologous protein other than a transgene described herein. In a specific embodiment, the APMV-8 strain is the APMV-8/Goose/Delaware/1053/76 strain.

In a specific embodiment, a transgene is incorporated into the genome of an APMV-9 strain, wherein the genome comprises the transcription units of the APMV-9 strain necessary for infection and replication of the virus in a substrate (e.g., a cell line susceptible to APMV-9 infection), subject (e.g., a human subject), or both. In a specific embodiment, a transgene is incorporated into the genome of an APMV-9 strain, wherein the genome comprises a transcription unit encoding the APMV-9 nucleocapsid (N) protein, a transcription unit encoding the APMV-9 phosphoprotein (P), a transcription unit encoding the APMV-9 matrix (M) protein, a transcription unit encoding the APMV-9 fusion (F) protein, a transcription unit encoding the APMV-9 hemagglutinin-neuraminidase (HN) protein, and a transcription unit encoding the APMV-9 large polymerase (L) protein. The transgene may be incorporated into the APMV-9 genome between two transcription units of an APMV-9 described herein (e.g., between the M and P transcription units or between the HN and L transcription units). In certain embodiments, the genome of the APMV-9 does not encode a heterologous protein other than a transgene described herein. In a specific embodiment, the APMV-9 strain is the APMV-9 duck/New York/22/1978 strain.

In a specific embodiment, a transgene is incorporated into the genome of an APMV-7 strain, wherein the genome comprises the transcription units of the APMV-7 strain necessary for infection and replication of the virus in a substrate (e.g., a cell line susceptible to APMV-7 infection), subject (e.g., a human subject), or both. In a specific embodiment, a transgene is incorporated into the genome of an APMV-7 strain, wherein the genome comprises a transcription unit encoding the APMV-7 nucleocapsid (N) protein, a transcription unit encoding the APMV-7 phosphoprotein (P), a transcription unit encoding the APMV-7 matrix (M) protein, a transcription unit encoding the APMV-7 fusion (F) protein, a transcription unit encoding the APMV-7 hemagglutinin-neuraminidase (HN) protein, and a transcription unit encoding the APMV-7 large polymerase (L) protein. The transgene may be incorporated into the APMV-7 genome between two transcription units of an APMV-7 described herein (e.g., between the M and P transcription units or between the HN and L transcription units). In certain embodiments, the genome of the APMV-7 does not encode a heterologous protein other than a transgene described herein. In a specific embodiment, the APMV-7 strain is the APMV-7/dove/Tennessee/4/75 strain.

In a specific embodiment, a transgene is incorporated into the genome of an APMV-2 strain, wherein the genome comprises the transcription units of the APMV-2 strain necessary for infection and replication of the virus in a substrate (e.g., a cell line susceptible to APMV-2 infection), subject (e.g., a human subject), or both. In a specific embodiment, a transgene is incorporated into the genome of an APMV-2 strain, wherein the genome comprises a transcription unit encoding the APMV-2 nucleocapsid (N) protein, a transcription unit encoding the APMV-2 phosphoprotein (P), a transcription unit encoding the APMV-2 matrix (M) protein, a transcription unit encoding the APMV-2 fusion (F) protein, a transcription unit encoding the APMV-2 hemagglutinin-neuraminidase (HN) protein, and a transcription unit encoding the APMV-2 large polymerase (L) protein. The transgene may be incorporated into the APMV-2 genome between two transcription units of an APMV-2 described herein (e.g., between the M and P transcription units or between the HN and L transcription units). In certain embodiments, the genome of the APMV-2 does not encode a heterologous protein other than a transgene described herein. In a specific embodiment, the APMV-2 strain is the APMV-2 Chicken/California/Yucaipa/1956 strain.

In a specific embodiment, a transgene is incorporated into the genome of an APMV-3 strain, wherein the genome comprises the transcription units of the APMV-3 strain necessary for infection and replication of the virus in a substrate (e.g., a cell line susceptible to APMV-3 infection), subject (e.g., a human subject), or both. In a specific embodiment, a transgene is incorporated into the genome of an APMV-3 strain, wherein the genome comprises a transcription unit encoding the APMV-3 nucleocapsid (N) protein, a transcription unit encoding the APMV-3 phosphoprotein (P), a transcription unit encoding the APMV-3 matrix (M) protein, a transcription unit encoding the APMV-3 fusion (F) protein, a transcription unit encoding the APMV-3 hemagglutinin-neuraminidase (HN) protein, and a transcription unit encoding the APMV-3 large polymerase (L) protein. The transgene may be incorporated into the APMV-3 genome between two transcription units of an APMV-3 described herein (e.g., between the M and P transcription units or between the HN and L transcription units). In certain embodiments, the genome of the APMV-3 does not encode a heterologous protein other than a transgene described herein. In a specific embodiment, the APMV-3 strain is the APMV-3 turkey/Wisconsin/68 strain.

In a specific embodiment, a transgene is incorporated into the genome of an APMV-6 strain, wherein the genome comprises the transcription units of the APMV-6 strain necessary for infection and replication of the virus in a substrate (e.g., a cell line susceptible to APMV-6 infection), subject (e.g., a human subject), or both. In a specific embodiment, a transgene is incorporated into the genome of an APMV-6 strain, wherein the genome comprises a transcription unit encoding the APMV-6 nucleocapsid (N) protein, a transcription unit encoding the APMV-6 phosphoprotein (P), a transcription unit encoding the APMV-6 matrix (M) protein, a transcription unit encoding the APMV-6 fusion (F) protein, a transcription unit encoding the APMV-6 hemagglutinin-neuraminidase (HN) protein, and a transcription unit encoding the APMV-6 large polymerase (L) protein. The transgene may be incorporated into the APMV-6 genome between two transcription units of an APMV-6 described herein (e.g., between the M and P transcription units or between the HN and L transcription units). In certain embodiments, the genome of the APMV-6 does not encode a heterologous protein other than a transgene described herein. In a specific embodiment, the APMV-6 strain is the APMV-6/duck/Hong Kong/18/199/77 strain.

### 5.1.2.2 Transgenes

In a specific embodiment, a transgene encoding a cytokine is incorporated into the genome of an APMV described herein. For example, the transgene may encode IL-2, IL-15Ra-IL-15, or GM-CSF. In another specific embodiment, a transgene encoding a tumor antigen is incorporated into the genome of an APMV described herein. For example, the transgene may encode a human papillomavirus (HPV) antigen, such as E6 or E7 (e.g., HPV-16 E6 or E7 protein) or other tumor antigens may be incorporated into the genome of an APMV described herein. See, e.g., Section 5.1.1 and Section 5.1.2.1, supra, for types and strains of APMV that may be used.

In certain embodiments, a transgene encoding a protein described herein (e.g., human IL-2, human IL-12, human GM-CSF, or human IL-15Ra-IL-15 protein, or a tumor antigen) comprises APMV regulatory signals (e.g., gene end, intergenic, and gene start sequences) and Kozak sequences. In some embodiments, a transgene encoding a protein described herein (e.g., human IL-2, human IL-12, human GM-CSF, human IL-15Ra-IL15 protein or tumor antigen) comprises APMV regulatory signals (e.g., gene end, intergenic, and gene start sequences), Kozak sequences and restriction sites to facilitate cloning. In certain embodiments, a transgene encoding a protein described herein (e.g., human IL-2, human IL-12, human GM-CSF, human IL-15Ra-IL15 protein or tumor antigen) comprises APMV regulatory signals (e.g., gene end, intergenic and gene start sequences), Kozak sequences, restriction sites to facilitate cloning, and additional nucleotides in the non-coding region to ensure compliance with the rule of six. In a preferred embodiment, the transgene complies with the rule of six.

IL-2

In a specific embodiment, a transgene encoding IL-2 is incorporated into the genome of an APMV described herein. See, e.g., Section 5.1.1 and Section 5.1.2.1, supra, for types and strains of APMV that may be used. In a specific embodiment, the transgene encodes human IL-2. One of skill in the art would be able to use such sequence information to produce a transgene for incorporation into the genome of an APMV described herein. For example, a transgene encoding a human IL-2 comprising the amino acid sequence set forth in GenBank No. NO_000577.2 may be incorporated into the genome of any APMV type or strain described herein. In a specific embodiment, such a transgene comprises the sequence set forth in SEQ ID NO:15. However, given the degeneracy of the nucleic acid code, there are a number of different nucleic acid sequences that may encode the same IL-2 protein. In a specific embodiment, a transgene comprising the nucleotide sequence encoding IL-2 (e.g., human IL-2) is codon optimized. See, e.g., Section 5.1.2.3, infra, for a discussion regarding codon optimization. In some embodiments, the transgene encoding a human IL-2 protein comprises the amino acid sequence encoded by the nucleic acid sequence comprising the sequence set forth in SEQ ID NO:15. The transgene encoding IL-2 (e.g., human IL-2) may be incorporated between any two APMV transcription units (e.g., between the APMV P and M transcription units, or between the HN and L transcription units).

“Interleukin-2” and “IL-2” refer to any IL-2 known to those of skill in the art. In certain embodiments, the IL-2 may be human, dog, cat, horse, pig, or cow IL-2. In a specific embodiment, the IL-2 is human IL-2. GenBank™ accession number NG_016779.1 (GI number 291219938) provides an exemplary human IL-2 nucleic acid sequence. GenBank™ accession number NP_000577.2 (GI number 28178861) provides an exemplary human IL-2 amino acid sequence. As used herein, the terms “interleukin-2” and “IL-2” encompass interleukin-2 polypeptides that are modified by post-translational processing such as signal peptide cleavage, disulfide bond formation, glycosylation (e.g., N-linked glycosylation), protease cleavage and lipid modification (e.g., S-palmitoylation). In some embodiments, IL-2 consists of a single polypeptide chain that includes a signal sequence. In other embodiments, IL-2 consists of a single polypeptide chain that does not include a signal sequence. The signal sequence can be the naturally occurring signal peptide sequence or a variant thereof. In some embodiments, the signal peptide is an IL-2 signal peptide. In some embodiments, the signal peptide is heterologous to an IL-2 signal peptide.

In a specific embodiment, a transgene encoding an IL-2 derivative is incorporated into the genome of an APMV described herein. See, e.g., Section 5.1.2.1, supra, for types and strains of APMV that may be used. In a specific embodiment, the transgene encodes a human IL-2 derivative. One of skill in the art would be able to use such sequence information to produce a transgene for incorporation into the genome of an APMV described herein. In a specific embodiment, an IL-2 derivative has at least 70%, 75%, 80%, 85%, 90%, 95%, 96%, 98%, or 99% amino acid sequence identity to an IL-2 known to those of skill in the art. Methods/techniques known in the art may be used to determine sequence identity (see, e.g., “Best Fit” or “Gap” program of the Sequence Analysis Software Package, version 10; Genetics Computer Group, Inc.). In a specific embodiment, an IL-2 derivative comprises deleted forms of a known IL-2 (e.g., human IL-2), wherein up to about 20, 15, 10, 9, 8, 7, 6, 5, 4, 3, 2 or 1 amino acid residues are deleted from the known IL-2 (e.g., human IL-2). Also provided herein are IL-2 derivatives comprising deleted forms of a known IL-2, wherein about 1-3, 3-5, 5-7, 7-10, 10-15, or 15-20 amino acid residues are deleted from the known IL-2 (e.g., human IL-2). Further provided herein are IL-2 derivatives comprising altered forms of a known IL-2 (e.g., human IL-2), wherein up to about 20, 15, 10, 9, 8, 7, 6, 5, 4, 3, 2 or 1 amino acid residues of the known IL-2 are substituted (e.g., conservatively substituted) with other amino acids. In a specific embodiment, the known IL-2 is human IL-2, such as, e.g., provided in GenBank™ accession number NP_000577.2 (GI number 28178861). In some embodiments, an IL-2 derivative comprises up to about 20, 15, 10, 9, 8, 7, 6, 5, 4, 3, 2 or 1 conservatively substituted amino acids. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophylic (Cys, Ser, Thr), acidic (Asp, Glu), basic (Asn, Gln, His, Lys, Arg), conformation disruptors (Gly, Pro) and aromatic (Trp, Tyr, Phe).

In a specific embodiment, an IL-2 derivative is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a native IL-2 (e.g., human IL-2). In another specific embodiment, an IL-2 derivative is a polypeptide encoded by a nucleic acid sequence that is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a nucleic acid sequence encoding a native IL-2. In a specific embodiment, the native IL-2 is human IL-2, such as, e.g., provided in GenBank™ accession number NP_000577.2 (GI number 28178861) or GenBank™ accession number NG_016779.1 (GI number 291219938). In another specific embodiment, an IL-2 derivative contains 1, 2, 3, 4, 5, 6, 7, 8, 9, 10, 11, 12, 13, 14, 15, 16, 17, 18, 19, 20 or more, or 2 to 5, 2 to 10, 5 to 10, 5 to 15, 5 to 20, 10 to 15, or 15 to 20 amino acid mutations (i.e., additions, deletions, substitutions or any combination thereof) relative to a native IL-2 (e.g., human IL-2). In another specific embodiment, an IL-2 derivative is a polypeptide encoded by nucleic acid sequence that can hybridize under high, moderate or typical stringency hybridization conditions to a nucleic acid sequence encoding a native IL-2 (e.g., human IL-2). Hybridization conditions are known to one of skill in the art (see, e.g., U.S. Patent Application No. 2005/0048549 at, e.g., paragraphs 72 and 73). In another specific embodiment, an IL-2 derivative is a polypeptide encoded by a nucleic acid sequence that can hybridize under high, moderate or typical stringency hybridization conditions to a nucleic acid sequence encoding a fragment of a native IL-2 (e.g., human IL-2) of at least 10 contiguous amino acids, at least 12 contiguous amino acids, at least 15 contiguous amino acids, at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, at least 150 contiguous amino acids, or 10 to 20, 20 to 50, 25 to 75, 25 to 100, 25 to 150, 50 to 75, 50 to 100, 75 to 100, 50 to 150, 75 to 150, 100 to 150, or 100 to 200 contiguous amino acids. In another specific embodiment, an IL-2 derivative is a fragment of a native IL-2 (e.g., human IL-2). IL-2 derivatives also include polypeptides that comprise the amino acid sequence of a naturally occurring mature form of IL-2 and a heterologous signal peptide amino acid sequence. In addition, IL-2 derivatives include polypeptides that have been chemically modified by, e.g., glycosylation, acetylation, pegylation, phosphorylation, amidation, derivitization by known protecting/blocking groups, proteolytic cleavage, linkage to a cellular ligand or other protein moiety, etc. Further, IL-2 derivatives include polypeptides comprising one or more non-classical amino acids. In specific embodiments, the IL-2 derivative retains one, two, or more, or all of the functions of the native IL-2 (e.g., human IL-2) from which it was derived. Examples of functions of IL-2 include regulation of signals to T cells, B cells, and NK cells, promotion of the development of T regulatory cells, and the maintenance of self-tolerance. Tests for determining whether or not an IL-2 derivative retains one or more functions of the native IL-2 (e.g., human IL-2) from which it was derived are known to one of skill in the art and examples are provided herein.

In specific embodiments, the transgene encoding IL-2 or a derivative thereof in a packaged genome of a recombinant APMV described herein is codon optimized.

IL-12

In a specific embodiment, a transgene encoding IL-12 is incorporated into the genome of an APMV described herein. See, e.g., Section 5.1.1 and 5.1.2.1, supra, for types and strains of APMV that may be used. In a specific embodiment, the transgene encodes human IL-12. One of skill in the art would be able to use such sequence information to produce a transgene for incorporation into the genome of an APMV described herein. For example, a transgene encoding human IL-12 comprising the amino acid sequence set forth in SEQ ID NO:34 may be incorporated into the genome of any APMV type or strain described herein. In a specific embodiment, such a transgene comprises the negative sense RNA transcribed from the nucleotide sequence set forth in SEQ ID NO:16. However, given the degeneracy of the nucleic acid code, there are a number of different nucleic acid sequences that may encode the same IL-12 protein. In a specific embodiment, a transgene comprising the nucleotide sequence encoding IL-12 (e.g., human IL-12) is codon optimized. See, e.g., Section 5.1.2.3, infra, for a discussion regarding codon optimization. In a specific embodiment, a transgene comprises the negative sense RNA transcribed from the codon optimized sequence set forth in SEQ ID NO:17. In some embodiments, the transgene encoding a human IL-12 protein comprises the amino acid sequence encoded by the nucleic acid sequence comprising the nucleotide sequence set forth in SEQ ID NO:16 or 17. The transgene encoding IL-12 (e.g., human IL-12) may be incorporated between any two APMV transcription units (e.g., between the APMV P and M transcription units, or between the HN and L transcription units).

“Interleukin-12” and “IL-12” refer to any IL-12 known to those of skill in the art. In certain embodiments, the IL-12 may be human, dog, cat, horse, pig, or cow IL-12. In a specific embodiment, the IL-12 is human IL-12. A typical IL-12 consists of a heterodimer encoded by two separate genes, IL-12A (the p35 subunit) and IL-12B (the p40 subunit), known to those of skill in the art. GenBank™ accession number NM_000882.3 (GI number 325974478) or SEQ ID NO:49 provides an exemplary human IL-12A nucleic acid sequence. GenBank™ accession number NM_002187.2 (GI number 24497437) or SEQ ID NO:47 provides an exemplary human IL-12B nucleic acid sequence. GenBank™ accession number NP_000873.2 (GI number 24430219) or SEQ ID NO:48 provides an exemplary human IL-12A (the p35 subunit) amino acid sequence. GenBank™ accession number NP_002178.2 (GI number 24497438) or SEQ ID NO:46 provides an exemplary human IL-12B (the p40 subunit) amino acid sequence. In certain embodiments, an IL-12 consists of a single polypeptide chain, comprising the p35 subunit and the p40 subunit, optionally separated by a linker sequence (such as, e.g., SEQ ID NO:35 (which is encoded by the nucleotide sequence set forth in SEQ ID NO:45)). In certain embodiments, an IL-12 consists of more than one polypeptide chain in quaternary association, e.g., p35 and p40. As used herein, the terms “interleukin-12” and “IL-12” encompass interleukin-12 polypeptides that are modified by post-translational processing such as signal peptide cleavage, disulfide bond formation, glycosylation (e.g., N-linked glycosylation), protease cleavage and lipid modification (e.g., S-palmitoylation). In some embodiments, one or both of the subunits of IL-12 or IL-12 consisting of a single polypeptide chain includes a signal sequence. In other embodiments, one or both of the subunits of IL-12 or IL-12 consisting of a single polypeptide chain does not include a signal sequence. The signal sequence can be the naturally occurring signal peptide sequence or a variant thereof. In some embodiments, the signal peptide is an IL-12 signal peptide. In some embodiments, the signal peptide is heterologous to an IL-12 signal peptide.

In specific embodiments, a polypeptide comprising the IL-12 p35 subunit and IL-12 p40 subunit directly fused to each other is functional (e.g., capable of specifically binding to the IL-12 receptor and inducing IL-12-mediated signal transduction and/or IL-12-mediated immune function). In a specific embodiment, the IL-12 p35 subunit and IL-12 p40 subunit or derivative(s) thereof are indirectly fused to each other using one or more linkers. Linkers suitable for preparing the IL-12 p35 subunit/p40 subunit fusion protein may comprise one or more amino acids (e.g., a peptide). In specific embodiments, a polypeptide comprising the IL-12 p35 subunit and IL-12 p40 subunit indirectly fused to each other using an amino acid linker (e.g., a peptide linker) is functional (e.g., capable of specifically binding to the IL-12 receptor and inducing IL-12-mediated signal transduction and/or IL-12-mediated immune function). In a specific embodiment, the linker is long enough to preserve the ability of the IL-12 p35 subunit and IL-12 p40 subunit to form a functional IL-12 heterodimer complex, which is capable of binding to the IL-12 receptor and inducing IL-12-mediated signal transduction. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is an amino acid sequence (e.g., a peptide) that is between 5 and 20 or 5 and 15 amino acids in length. In certain embodiments, an IL-12 encoded by a transgene in a packaged genome of a recombinant APMV described herein consists of more than one polypeptide chain in quaternary association, e.g., a polypeptide chain comprising the IL-12 p35 subunit or a derivative thereof in quaternary association with a polypeptide chain comprising the IL-12 p40 subunit or a derivative thereof. In certain embodiments, the linker is the amino acid sequence set forth in SEQ ID NO:35. In certain embodiments, the elastin-like polypeptide sequence comprises the amino acid sequence VPGXG (SEQ ID NO:22), wherein X is any amino acid except proline. In certain embodiments, the elastin-like polypeptide sequence comprises the amino acid sequence VPGXGVPGXG (SEQ ID NO:23), wherein X is any amino acid except proline. In certain embodiments, the linker may be a linker described in U.S. Pat. No. 5,891,680, which is incorporated by reference herein in its entirety.

In a specific embodiment, a transgene encoding an IL-12 derivative is incorporated into the genome of an APMV described herein. See, e.g., Section 5.1.2.1, supra, for types and strains of APMV that may be used. In a specific embodiment, the transgene encodes a human IL-12 derivative. One of skill in the art would be able to use such sequence information to produce a transgene for incorporation into the genome of an APMV described herein. In a specific embodiment, an IL-12 derivative has at least 70%, 75%, 80%, 85%, 90%, 95%, 96%, 98%, or 99% amino acid sequence identity to an IL-12 known to those of skill in the art. Methods/techniques known in the art may be used to determine sequence identity (see, e.g., “Best Fit” or “Gap” program of the Sequence Analysis Software Package, version 10; Genetics Computer Group, Inc.). In a specific embodiment, an IL-12 derivative comprises deleted forms of a known IL-12, wherein up to about 20, 15, 10, 9, 8, 7, 6, 5, 4, 3, 2 or 1 amino acid residues are deleted from the known IL-12. Also provided herein are IL-12 derivatives comprising deleted forms of a known IL-12, wherein about 1-3, 3-5, 5-7, 7-10, 10-15, or 15-20 amino acid residues are deleted from the known IL-12. Further provided herein are IL-12 derivatives comprising altered forms of a known IL-12, wherein up to about 20, 15, 10, 9, 8, 7, 6, 5, 4, 3, 2 or 1 amino acid residues of the known IL-12 are substituted (e.g., conservatively substituted) with other amino acids. In some embodiments, the IL-12 derivative comprises up to about 20, 15, 10, 9, 8, 7, 6, 5, 4, 3, 2 or 1 conservatively substituted amino acids (see, e.g., Huang et al., 2016, Preclinical validation:LV/IL-12 transduction of patient leukemia cells for immunotherapy of AML, Molecular Therapy—Methods & Clinical Development, 3, 16074; doi:10.1038/mtm.2016.74, which is incorporated by reference herein in its entirety). In some embodiments, the conservatively substituted amino acids are not projected to be in the cytokine/receptor interface (see, e.g., Huang et al., 2016, Preclinical validation:LV/IL-12 transduction of patient leukemia cells for immunotherapy of AML, Molecular Therapy—Methods & Clinical Development, 3, 16074; doi:10.1038/mtm.2016.74; Jones & Vignali, 2011, Molecular Interactions within the IL-6/IL-12 cytokine/receptor superfamily, Immunol Res., 51(1):5-14, doi:10.1007/s12026-011-8209-y; each of which is incorporated by reference herein in its entirety). In some embodiments, the IL-12 derivative comprises an IL-12 p35 subunit having the amino acid substitution L165S (i.e., leucine at position 165 of the IL-12 p35 subunit in the IL-12 derivative is substituted with a serine). In some embodiments, the IL-12 derivative comprises an IL-12 p40 subunit having the amino acid substitution of C2G (i.e., cysteine at position 2 of the immature IL-12 p40 subunit (i.e., the IL-12 p40 subunit containing the signal peptide) in the IL-12 derivative is substituted with a glycine).

In a specific embodiment, an IL-12 derivative comprises an IL-12 p35 subunit that is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a native IL-12 p35 subunit (e.g., a human IL-12 p35 subunit). In another specific embodiment, an IL-12 derivative is a polypeptide encoded by a nucleic acid sequence, wherein a portion of nucleic acid sequences encodes an IL-12 p35 subunit, wherein said the nucleic acid sequence of said portion is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a nucleic acid sequence encoding a native IL-12 p35 subunit (e.g., a human IL-12 p35 subunit). In a specific embodiment, an IL-12 derivative comprises an IL-12 p40 subunit that is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a native IL-12 p40 subunit (e.g., a human IL-12 p40 subunit). In another specific embodiment, an IL-12 derivative is a polypeptide encoded by a nucleic acid sequence, wherein a portion of nucleic acid sequence encodes an IL-12 p40 subunit, wherein said the nucleic acid sequence of said portion is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a nucleic acid sequence encoding a native IL-12 p40 subunit (e.g., a human IL-12 p40 subunit). In another specific embodiment, an IL-12 derivative comprises an IL-12 p35 subunit, an IL-12 p40 subunit, or both containing 1, 2, 3, 4, 5, 6, 7, 8, 9, 10, 11, 12, 13, 14, 15, 16, 17, 18, 19, 20 or more, or 2 to 5, 2 to 10, 5 to 10, 5 to 15, 5 to 20, 10 to 15, or 15 to 20 amino acid mutations (i.e., additions, deletions, substitutions or any combination thereof) relative to a native IL-12 p35 subunit, a native IL-12 p40 subunit, or both. In another specific embodiment, an IL-12 derivative is a polypeptide encoded by nucleic acid sequence that can hybridize under high, moderate or typical stringency hybridization conditions to a nucleic acid sequence encoding a native IL-12 p35 subunit, a native IL-12 p40 subunit, or both. Hybridization conditions are known to one of skill in the art (see, e.g., U.S. Patent Application No. 2005/0048549 at, e.g., paragraphs 72 and 73). In another specific embodiment, an IL-12 derivative is a polypeptide encoded by a nucleic acid sequence that can hybridize under high, moderate or typical stringency hybridization conditions to a nucleic acid sequence encoding a fragment of a native IL-12 p35 subunit, a fragment of a native IL-12 p40 subunit, or fragments of both of a native IL-12 p35 subunit and a native IL-12 p40 subunit, wherein the fragment(s) is at least 10 contiguous amino acids, at least 12 contiguous amino acids, at least 15 contiguous amino acids, at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, at least 150 contiguous amino acids, or 10 to 20, 20 to 50, 25 to 75, 25 to 100, 25 to 150, 50 to 75, 50 to 100, 75 to 100, 50 to 150, 75 to 150, 100 to 150, or 100 to 200 contiguous amino acids. In another specific embodiment, an IL-12 derivative comprises a fragment of a native IL-12 p35 subunit, a native IL-12 p40 subunit, or both. In another specific embodiment, an IL-12 derivative comprises a fragment of native IL-12 p35 subunit, a fragment of native IL-12 p40 subunit, or both. In another specific embodiment, an IL-12 derivative comprises a subunit (e.g., p35 or p40) encoded by a nucleotide sequence that hybridizes over its full length to the nucleotide encoding the native subunit (e.g., native p40 subunit or native p35 subunit). In a specific embodiment, an IL-12 derivative comprises a native IL-12 p40 subunit and a derivative of an IL-12 p35 subunit. In a specific embodiment, the IL-12 derivative comprises a native IL-12 p35 subunit and a derivative of an IL-12 p40 subunit. IL-12 derivatives also include polypeptides that comprise the amino acid sequence of a naturally occurring mature form of IL-12 and a heterologous signal peptide amino acid sequence. In addition, IL-12 derivatives include polypeptides that have been chemically modified by, e.g., glycosylation, acetylation, pegylation, phosphorylation, amidation, derivitization by known protecting/blocking groups, proteolytic cleavage, linkage to a cellular ligand or other protein moiety, etc. Further, IL-12 derivatives include polypeptides comprising one or more non-classical amino acids. In specific embodiments, the IL-12 derivative retains one, two, or more, or all of the functions of the native IL-12 from which it was derived. Examples of functions of IL-12 include the promotion of the development of T helper 1 cells and the activation of pro-inflammatory immune response pathways. Tests for determining whether or not an IL-12 derivative retains one or more functions of the native IL-12 (e.g., human IL-12) from which it was derived are known to one of skill in the art and examples are provided herein.

In specific embodiments, the transgene encoding IL-12 or a derivative thereof in a packaged genome of a recombinant APMV described herein is codon optimized. In a specific embodiment, the nucleotide sequence(s) encoding one or both subunits of a native IL-12 may be codon optimized. A nonlimiting example of a codon-optimized sequence encoding IL-12 includes SEQ ID NO:17.

IL-15Ra-IL-15

In a specific embodiment, a transgene encoding IL-15Ra-IL-15 is incorporated into the genome of an APMV described herein. See, e.g., Section 5.1.1 and 5.1.2.1, supra, for types and strains of APMV that may be used. In a specific embodiment, the transgene encodes human IL-15Ra-IL-15. One of skill in the art would be able to use such sequence information to produce a transgene for incorporation into the genome of an APMV described herein. For example, a transgene encoding a human IL-15Ra-IL-15 comprising the amino sequence set forth in SEQ ID NO:37 may be incorporated into the genome of any APMV type or strain described herein. In a specific embodiment, such a transgene comprises the negative sense RNA transcribed from the nucleotide sequence set forth in SEQ ID NO:18. However, given the degeneracy of the nucleic acid code, there are a number of different nucleic acid sequences that may encode the same IL-15Ra-IL-15 protein. In a specific embodiment, a transgene comprising the nucleotide sequence encoding IL-15Ra-IL-15 (e.g., human IL-15Ra-IL-15) is codon optimized. See, e.g., Section 5.1.2.3, infra, for a discussion regarding codon optimization. In some embodiments, the transgene encoding a human IL-15Ra-IL-15 protein comprises the amino acid sequence encoded by the nucleic acid sequence comprising the sequence set forth in SEQ ID NO:18. The transgene encoding IL-15Ra-IL-15 (e.g., human IL-15Ra-IL-15) may be incorporated between any two APMV transcription units (e.g., between the APMV P and M transcription units, or between the HN and L transcription units).

As used herein, the term “IL-15Ra-IL-15” refers to a complex comprising IL-15 or a derivative thereof and IL-15Ra or a derivative thereof covalently or noncovalently bound to each other. In a specific embodiment, IL-15Ra or a derivative thereof has a relatively high affinity for IL-15 or a derivative thereof, e.g., Ka of 10 to 50 pM as measured by a technique known in the art, e.g., KinEx A assay, plasma surface resonance (e.g., BIAcore assay). In a preferred embodiment, the IL-15Ra-IL-15 induces IL-15-mediated signal transduction, as measured by assays well-known in the art, e.g., electromobility shift assays, ELISAs and other immunoassays. In some embodiments, the IL-15Ra-IL-15 complex retains the ability to specifically bind to the βγ chain. In a preferred embodiment, the IL-15Ra-IL-15 complex retains the ability to specifically bind to the βγ chain and induce/mediate IL-15 signal transduction.

In specific embodiments, the IL-15Ra-IL-15 (e.g., human IL-15Ra-IL-15) may be formed by directly fusing IL-15Ra or a derivative thereof (e.g., human IL-15Ra or a derivative thereof) to IL-15 or a derivative thereof (e.g., human IL-15 or a derivative thereof), using either non-covalent bonds or covalent bonds (e.g., by combining amino acid sequences via peptide bonds). In specific embodiments, the IL-15Ra-IL-15 (e.g., human IL-15Ra-IL-15) may be formed by indirectly fusing IL-15Ra or a derivative thereof (e.g., human IL-15Ra or a derivative thereof) to IL-15 or a derivative thereof (e.g., human IL-15 or a derivative thereof) using one or more linkers. Linkers suitable for preparing the IL-15Ra-IL-15 (e.g., human IL-15Ra-IL-15) comprise peptides, alkyl groups, chemically substituted alkyl groups, polymers, or any other covalently-bonded or non-covalently bonded chemical substance capable of binding together two or more components. Polymer linkers comprise any polymers known in the art, including polyethylene glycol (“PEG”). In some embodiments, the linker is a peptide that is 1, 2, 3, 4, 5,6, 7, 8, 9, 10, 11, 12, 13, 14, 15, 16, 17, 18, 19, 20 or more amino acids long. In a specific embodiment, the linker is long enough to preserve the ability of IL-15 or a derivative thereof (e.g., human IL-15 or a derivative thereof) to bind to the IL-15Ra or a derivative thereof (e.g., human IL-15Ra or a derivative thereof). In other embodiments, the linker is long enough to preserve the ability of the IL-15Ra-IL-15 complex to bind to the fly receptor complex and to act as an agonist to mediate IL-15 signal transduction. In certain embodiments, the linker has the amino acid sequence set forth in SEQ ID NO:36 (the nucleotide sequence encoding such a linker sequence is set forth in SEQ ID NO:42).

In certain embodiments, the IL-15Ra-IL-15 (e.g., human IL-15Ra-IL-15) comprises the signal sequence of IL-15 (e.g., human IL-15). In other embodiments, the IL-15Ra-IL-15 (e.g., human IL-15Ra-IL-15) comprises the signal sequence of IL-15Ra (e.g., human IL-15Ra). In yet other embodiments, the IL-15Ra-IL-15 (e.g., human IL-15Ra-IL-15) comprises a signal sequence heterologous to IL-15 (e.g., human IL-15) and IL-15Ra (e.g., human IL-15Ra). In a specific embodiment, the IL-15Ra-IL-15 (e.g., human IL-15Ra-IL-15) comprises the signal sequence set forth in SEQ ID NO:41 (the nucleotide sequence encoding such a signal sequence is set forth in SEQ ID NO:43).

In a specific embodiment, an IL-15Ra-IL-15 (e.g., human IL-15Ra-IL-15) comprises a signal sequence, a tag (e.g., a flag tag), a soluble form of IL-15Ra (e.g., the IL-15Ra sushi domain), a linker, and IL-15. In another specific embodiment, a human IL-15Ra-IL-15 comprises an amino acid sequence comprising: (1) a signal sequence comprising (consisting of) the amino acid sequence set forth in SEQ ID NO:41; (2) a flag-tag comprising (consisting of) the amino acid sequence set forth in SEQ ID NO:38; (3) a soluble form of human IL-15Ra comprising (consisting of) the amino acid sequence set forth in SEQ ID NO:39; (4) a linker comprising (consisting of) the amino acid sequence set forth in SEQ ID NO:36; and (5) human IL-15 comprising (consisting of) the amino acid sequence set forth in SEQ ID NO:40. Due to the degeneracy of the nucleic acid code, there are a number of different nucleic acid sequences that may encode the same human IL-15Ra-IL-15 protein. In another specific embodiment, a human IL-15Ra-IL-15 comprises: (1) a signal sequence encoded by a nucleotide sequence comprising (consisting of) the nucleotide sequence set forth in SEQ ID NO:43; (2) a flag-tag encoded by a nucleotide sequence comprising (consisting of) the nucleotide sequence set forth in SEQ ID NO:44; (3) a soluble form of human IL-15Ra encoded by a nucleotide sequence comprising (consisting of) the nucleotide sequence set forth in SEQ ID NO:50; (4) a linker encoded by a nucleotide sequence comprising (consisting of) the nucleotide sequence set forth in SEQ ID NO:42; and (5) human IL-15 encoded by a nucleotide sequence comprising (consisting of) the nucleotide sequence set forth in SEQ ID NO:51.

As used herein, the terms “interleukin-15” and “IL-15” refers to any IL-15 known to those of skill in the art. In certain embodiments, the IL-15 may be human, dog, cat, horse, pig, or cow IL-15. Examples of GeneBank Accession Nos. for the amino acid sequence of various species of IL-15 include NP_000576 (human, immature form), CAA62616 (human, immature form), NP_001009207 (Felis catus, immature form), AAB94536 (rattus, immature form), AAB41697 (rattus, immature form), NP_032383 (Mus musculus, immature form), AAR19080 (canine), AAB60398 (macaca mulatta, immature form), AAI00964 (human, immature form), AAH23698 (mus musculus, immature form), and AAH18149 (human). Examples of GeneBank Accession Nos. for the nucleotide sequence of various species of IL-15 include NM_000585 (human), NM_008357 (Mus musculus), and RNU69272 (rattus norvegicus). As used herein, the terms “interleukin-15” and “IL-15” encompass interleukin-15 polypeptides that are modified by post-translational processing such as signal peptide cleavage, disulfide bond formation, glycosylation (e.g., N-linked glycosylation), protease cleavage and lipid modification (e.g., S-palmitoylation). In some embodiments, IL-15 consists of a single polypeptide chain that includes a signal sequence. In other embodiments, IL-15 consists of a single polypeptide chain that does not include a signal sequence.

In a specific embodiment, the human IL-15 component of the human IL-15Ra-IL-15 sequence comprises the amino acid sequence set forth in SEQ ID NO:40. In some embodiments, the human IL-15 component of the human IL-15Ra-IL-15 comprises the nucleotide sequence set forth in SEQ ID NO:51. However, given the degeneracy of the nucleic acid code, there are a number of different nucleic acid sequences that may encode the same IL-15 protein. In a specific embodiment, the nucleotide sequence encoding human IL-15 component of the human IL-15Ra-IL15 transgene is codon optimized. See, e.g., Section 5.1.2.3, infra, for a discussion regarding codon optimization.

In a specific embodiment, the IL-15 (e.g., human IL-15) component of the IL-15Ra-IL-15 (e.g., human IL-15Ra-IL-15) sequence is an IL-15 derivative. In a specific embodiment, an IL-15 derivative has at least 70%, 75%, 80%, 85%, 90%, 95%, 96%, 98%, or 99% amino acid sequence identity to an IL-15 known to those of skill in the art. Methods/techniques known in the art may be used to determine sequence identity (see, e.g., “Best Fit” or “Gap” program of the Sequence Analysis Software Package, version 10; Genetics Computer Group, Inc.). In a specific embodiment, an IL-15 derivative comprises deleted forms of a known IL-15 (e.g., human IL-15), wherein up to about 20, 15, 10, 9, 8, 7, 6, 5, 4, 3, 2 or 1 amino acid residues are deleted from the known IL-15. Also provided herein are IL-15 derivatives comprising deleted forms of a known IL-15 (e.g., human IL-15), wherein about 1-3, 3-5, 5-7, 7-10, 10-15, or 15-20 amino acid residues are deleted from the known IL-15. Further provided herein are IL-15 derivatives comprising altered forms of a known IL-15 (e.g., human IL-15), wherein up to about 20, 15, 10, 9, 8, 7, 6, 5, 4, 3, 2 or 1 amino acid residues of the known IL-15 are substituted (e.g., conservatively substituted) with other amino acids. In some embodiments, an IL-15 derivative comprises up to about 20, 15, 10, 9, 8, 7, 6, 5, 4, 3, 2 or 1 conservatively substituted amino acids. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophylic (Cys, Ser, Thr), acidic (Asp, Glu), basic (Asn, Gln, His, Lys, Arg), conformation disruptors (Gly, Pro) and aromatic (Trp, Tyr, Phe).

In a specific embodiment, an IL-15 derivative is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a native IL-15 (e.g., human IL-15). In another specific embodiment, an IL-15 derivative is a polypeptide encoded by a nucleic acid sequence that is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a nucleic acid sequence encoding a native IL-15 (e.g., human IL-15). In another specific embodiment, an IL-15 derivative contains 1, 2, 3, 4, 5, 6, 7, 8, 9, 10, 11, 12, 13, 14, 15, 16, 17, 18, 19, 20 or more, or 2 to 5, 2 to 10, 5 to 10, 5 to 15, 5 to 20, 10 to 15, or 15 to 20 amino acid mutations (i.e., additions, deletions, substitutions, or any combination thereof) relative to a native IL-15 (e.g., human IL-15). In another specific embodiment, an IL-15 derivative is a polypeptide encoded by nucleic acid sequence that can hybridize under high, moderate or typical stringency hybridization conditions to a nucleic acid sequence encoding a native IL-15 (e.g., human IL-15). Hybridization conditions are known to one of skill in the art (see, e.g., U.S. Patent Application No. 2005/0048549 at, e.g., paragraphs 72 and 73). In another specific embodiment, an IL-15 derivative is a polypeptide encoded by a nucleic acid sequence that can hybridize under high, moderate or typical stringency hybridization conditions to a nucleic acid sequence encoding a fragment of a native IL-15 (e.g., human IL-15) of at least 10 contiguous amino acids, at least 12 contiguous amino acids, at least 15 contiguous amino acids, at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, at least 150 contiguous amino acids, or 10 to 20, 20 to 50, 25 to 75, 25 to 100, 25 to 150, 50 to 75, 50 to 100, 75 to 100, 50 to 150, 75 to 150, 100 to 150, or 100 to 200 contiguous amino acids. In another specific embodiment, an IL-15 derivative is a fragment of a native IL-15 (e.g., human IL-15). IL-15 derivatives also include polypeptides that comprise the amino acid sequence of a naturally occurring mature form of IL-15 and a heterologous signal peptide amino acid sequence. In addition, IL-15 derivatives include polypeptides that have been chemically modified by, e.g., glycosylation, acetylation, pegylation, phosphorylation, amidation, derivitization by known protecting/blocking groups, proteolytic cleavage, linkage to a cellular ligand or other protein moiety, etc. Further, IL-15 derivatives include polypeptides comprising one or more non-classical amino acids. In specific embodiments, the IL-15 derivative retains one, two, or more, or all of the functions of the native IL-15 (e.g., human IL-15) from which it was derived. Examples of functions of IL-15 include the development and differentiation of NK cells and promotion of the survival and expansion of memory CD8+ T cells. Tests for determining whether or not an IL-15 derivative retains one or more functions of the native IL-15 (e.g., human IL-15) from which it was derived are known to one of skill in the art and examples are provided herein.

As used herein, the terms “IL-15Ra” and “interleukin-15 receptor alpha” refers to any IL-15Ra known to those of skill in the art. In certain embodiments, the IL-15 may be human, dog, cat, horse, pig, or cow IL-15Ra. Examples of GeneBank Accession Nos. for the amino acid sequence of various native mammalian IL-15Ra include NP_002180 (human), ABK41438 (Macaca mulatta), NP_032384 (Mus musculus), Q60819 (Mus musculus), CAI41082 (human). Examples of GeneBank Accession Nos. for the nucleotide sequence of various species of native mammalian IL-15Ra include NM_002189 (human), EF033114 (Macaca mulatta), and NM_008358 (Mus musculus). In a specific embodiment, the IL-15Ra is soluble.

As used herein, the terms “interleukin-15 receptor alpha” and “IL-15Ra” encompass IL-15Ra polypeptides that are modified by post-translational processing such as signal peptide cleavage, disulfide bond formation, glycosylation (e.g., N-linked glycosylation), protease cleavage and lipid modification (e.g., S-palmitoylation). In some embodiments, IL-15Ra consists of a single polypeptide chain that includes a signal sequence. In other embodiments, IL-15Ra consists of a single polypeptide chain that does not include a signal sequence. The signal sequence can be the naturally occurring signal peptide sequence or a variant thereof. In some embodiments, the signal peptide is an IL-15Ra signal peptide.

In a specific embodiment, the IL-15Ra component of the IL-15Ra-IL-15 sequence comprises a human IL-15Ra derivative. In a specific embodiment, an IL-15Ra derivative has at least 70%, 75%, 80%, 85%, 90%, 95%, 96%, 98%, or 99% amino acid sequence identity to an IL-15Ra known (e.g., a human IL-15Ra) to those of skill in the art. Methods/techniques known in the art may be used to determine sequence identity (see, e.g., “Best Fit” or “Gap” program of the Sequence Analysis Software Package, version 10; Genetics Computer Group, Inc.). In a specific embodiment, an IL-15Ra derivative comprises deleted forms of a known IL-15Ra, wherein up to about 20, 15, 10, 9, 8, 7, 6, 5, 4, 3, 2 or 1 amino acid residues are deleted from the known IL-15Ra (e.g., a human IL-15Ra). Also provided herein are IL-15Ra derivatives comprising deleted forms of a known IL-15Ra (e.g., a human IL-15Ra), wherein about 1-3, 3-5, 5-7, 7-10, 10-15, or 15-20 amino acid residues are deleted from the known IL-15Ra. Further provided herein are IL-15Ra derivatives comprising altered forms of a known IL-15Ra, wherein up to about 20, 15, 10, 9, 8, 7, 6, 5, 4, 3, 2 or 1 amino acid residues of the known IL-15Ra are substituted (e.g., conservatively substituted) with other amino acids. In some embodiments, an IL-15Ra derivative comprises up to about 20, 15, 10, 9, 8, 7, 6, 5, 4, 3, 2 or 1 conservatively substituted amino acids. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophylic (Cys, Ser, Thr), acidic (Asp, Glu), basic (Asn, Gln, His, Lys, Arg), conformation disruptors (Gly, Pro) and aromatic (Trp, Tyr, Phe).

In a specific embodiment, an IL-15Ra derivative is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a native IL-15Ra. In another specific embodiment, an IL-15Ra derivative is a polypeptide encoded by a nucleic acid sequence that is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a nucleic acid sequence encoding a native IL-15Ra. In another specific embodiment, an IL-15Ra derivative contains 1, 2, 3, 4, 5, 6, 7, 8, 9, 10, 11, 12, 13, 14, 15, 16, 17, 18, 19, 20 or more, or 2 to 5, 2 to 10, 5 to 10, 5 to 15, 5 to 20, 10 to 15, or 15 to 20 amino acid mutations (i.e., additions, deletions and/or substitutions) relative to a native IL-15Ra. In another specific embodiment, an IL-15Ra derivative is a polypeptide encoded by nucleic acid sequence that can hybridize under high, moderate or typical stringency hybridization conditions to a nucleic acid sequence encoding a native IL-15Ra. Hybridization conditions are known to one of skill in the art (see, e.g., U.S. Patent Application No. 2005/0048549 at, e.g., paragraphs 72 and 73). In another specific embodiment, an IL-15Ra derivative is a polypeptide encoded by a nucleic acid sequence that can hybridize under high, moderate or typical stringency hybridization conditions to a nucleic acid sequence encoding a fragment of a native IL-15Ra of at least 10 contiguous amino acids, at least 12 contiguous amino acids, at least 15 contiguous amino acids, at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, at least 150 contiguous amino acids, or 10 to 20, 20 to 50, 25 to 75, 25 to 100, 25 to 150, 50 to 75, 50 to 100, 75 to 100, 50 to 150, 75 to 150, 100 to 150, or 100 to 200 contiguous amino acids.

In a preferred embodiment, a derivative of IL-15Ra is a soluble form of IL-15Ra that lacks the transmembrane domain of IL-15Ra, and optionally, lacks the intracellular domain of native IL-15Ra. In a particular embodiment, a derivative of IL-15Ra consists of the extracellular domain of IL-15Ra and lacks the transmembrane and intracellular domains of IL-15Ra. In another embodiment, a derivative of IL-15Ra is a soluble form of IL-15Ra that comprises (consists of) the extracellular domain of IL-15Ra or a fragment thereof In certain embodiments, a derivative of IL-15Ra is a soluble form of IL-15Ra that comprises (consists of) a fragment of the extracellular domain comprising the sushi domain or exon 2 of native IL-15Ra. In certain embodiments, a derivative of IL-15Ra is a soluble form of IL-15Ra that comprises (consists of) the sushi domain or exon 2 of native IL-15Ra. In some embodiments, a derivative of IL-15Ra is a soluble form of IL-15Ra that comprises (consists of) a fragment of the extracellular domain comprising the sushi domain or exon 2 of IL-15Ra and at least one amino acid that is encoded by exon 3. In certain embodiments, a derivative of IL-15Ra is a soluble form of IL-15Ra that comprises (consists of) a fragment of the extracellular domain comprising the sushi domain or exon 2 of IL-15Ra and an IL-15Ra hinge region or a fragment thereof.

In another specific embodiment, an IL-15Ra derivative is a fragment of a native IL-15Ra. IL-15Ra derivatives also include polypeptides that comprise the amino acid sequence of a naturally occurring mature form of IL-15Ra and a heterologous signal peptide amino acid sequence. In addition, IL-15Ra derivatives include polypeptides that have been chemically modified by, e.g., glycosylation, acetylation, pegylation, phosphorylation, amidation, derivitization by known protecting/blocking groups, proteolytic cleavage, linkage to a cellular ligand or other protein moiety, etc. Further, IL-15Ra derivatives include polypeptides comprising one or more non-classical amino acids. In specific embodiments, the IL-15Ra derivative retains one, two, or more, or all of the functions of the native IL-15Ra from which it was derived. Examples of functions of IL-15Ra include enhancing cell proliferation and the expression of an apoptosis inhibitor. Tests for determining whether or not an IL-15Ra derivative retains one or more functions of the native IL-15Ra from which it was derived are known to one of skill in the art and examples are provided herein.

In a specific embodiment, the human IL-15Ra component of the human IL-15Ra-IL-15 sequence comprises (consists of) the amino acid sequence set forth in SEQ ID NO:39. In some embodiments, the human IL-15Ra component of the human IL-15Ra-IL-15 comprises (consists of) the nucleotide sequence set forth in SEQ ID NO:50. However, given the degeneracy of the nucleic acid code, there are a number of different nucleic acid sequences that may encode the same human IL-15Ra protein. In a specific embodiment, the nucleotide sequence encoding the human IL-15Ra is codon optimized. See, e.g., Section 5.1.2.3, infra, for a discussion regarding codon optimization.

Tumor Antigens

In a specific embodiment, a transgene encoding a tumor antigen (e.g., HPV-16 E6 or E7 protein) is incorporated into the genome of an APMV described herein. See, e.g., Section 5.1.1 and Section 5.1.2.1, supra, for types and strains of APMV that may be used. In a specific embodiment, a transgene encoding an HPV-16 E6 protein may be incorporated into the genome of an APMV described herein. An exemplary amino acid sequence for HPV-16 E6 protein includes GenBank Accession No. AKN79013.1. An exemplary nucleic acid sequence encoding the HPV-16 E6 protein includes GenBank Accession No. KP677555.1. One of skill in the art would be able to use such sequence information to produce a transgene for incorporation into the genome of an APMV described herein. For example, a transgene encoding an HPV16 E-6 protein comprising the amino acid sequence set forth in GenBank Accession No. AKN79013.1 may be incorporated into the genome of any APMV type or strain described herein. In a specific embodiment, such a transgene comprises the negative sense RNA transcribed from the nucleotide sequence set forth in SEQ ID NO:19. However, given the degeneracy of the nucleic acid code, there are a number of different nucleic acid sequences that may encode the same HPV-E6 protein. In a specific embodiment, a transgene comprising the nucleotide sequence encoding HPV-16 E6 protein is codon optimized. See, e.g., Section 5.1.2.3, infra, for a discussion regarding codon optimization. In some embodiments, the transgene encoding HPV-16 E6 protein comprises the amino acid sequence encoded by the nucleic acid sequence comprising the nucleotide sequence set forth in SEQ ID NO:19. The transgene encoding HPV-16 E6 protein may be incorporated between any two APMV transcription units (e.g., between the APMV P and M transcription units, or between the HN and L transcription units).

In a specific embodiment, a transgene encoding an HPV-16 E7 protein may be incorporated into the genome of an APMV described herein. An exemplary amino acid sequence for HPV-16 E7 protein includes GenBank Accession No. AIQ82815.1. An exemplary nucleic acid sequence encoding the HPV-16 E7 protein includes GenBank Accession No. KM058635.1. One of skill in the art would be able to use such sequence information to produce a transgene for incorporation into the genome of an APMV described herein. For example, a transgene encoding an HPV16 E-7 protein comprising the amino acid sequence set forth in GenBank Accession No. AIQ82815.1 may be incorporated into the genome of any APMV type or strain described herein. In a specific embodiment, such a transgene comprises the negative sense RNA transcribed from the nucleotide sequence set forth in SEQ ID NO:20. However, given the degeneracy of the nucleic acid code, there are a number of different nucleic acid sequences that may encode the same HPV-16 E7 protein. In a specific embodiment, a transgene comprising the nucleotide sequence encoding HPV-16 E7 protein is codon optimized. See, e.g., Section 5.1.2.3, infra, for a discussion regarding codon optimization. In some embodiments, the transgene encoding HPV-16 E7 protein comprises the amino acid sequence encoded by the nucleic acid sequence comprising the sequence set forth in SEQ ID NO:20. The transgene encoding HPV-16 E7 protein may be incorporated between any two APMV transcription units (e.g., between the APMV P and M transcription units, or between the HN and L transcription units).

GM-CSF

In a specific embodiment, a transgene encoding granulocyte-macrophage colony-stimulating factor (GM-CSF; e.g., human GM-CSF) is incorporated into the genome of an APMV described herein. See, e.g., Section 5.1.1 and Section 5.1.2.1, supra, for types and strains of APMV that may be used. In a specific embodiment, the transgene encodes human GM-CSF. One of skill in the art would be able to use such sequence information to produce a transgene for incorporation into the genome of an APMV described herein. For example, a transgene encoding a human GM-CSF comprising the amino acid sequence set forth in GenBank Accession No. X03021.1 may be incorporated into the genome of any APMV type or strain described herein. In a specific embodiment, such a transgene comprises the negative sense RNA transcribed from the nucleotide sequence set forth in SEQ ID NO:21. However, given the degeneracy of the nucleic acid code, there are a number of different nucleic acid sequences that may encode the same GM-CSF protein. In a specific embodiment, a transgene comprising the nucleotide sequence encoding GM-CSF (e.g., human GM-CSF) is codon optimized. See, e.g., Section 5.1.2.3, infra, for a discussion regarding codon optimization. In some embodiments, the transgene encoding a human GM-CSF protein comprises the amino acid sequence encoded by the nucleic acid sequence comprising the sequence set forth in SEQ ID NO:21. The transgene encoding GM-CSF (e.g. human GM-CSF) may be incorporated between any two APMV transcription units (e.g., between the APMV P and M transcription units, or between the HN and L transcription units).

As used herein, the terms “granulocyte-macrophage colony-stimulating factor” and “GM-CSF” refers to any GM-CSF known to those of skill in the art. In certain embodiments, the GM-CSF may be human, dog, cat, horse, pig, or cow GM-CSF. Examples of GeneBank Accession Nos. for the amino acid sequence of various species of GM-CSF include NP_000749.2 (human, precursor), AAA52578.1 (human), AAC06041.1 (Felis catus), NP_446304.1 (rattus norvegicus, precursor), NP_034099.2 (mus musculus, precursor), CAA26820.1 (mus musculus), AAB19466.1 (canine), AAG16626.1 (macaca mulatta, immature form), and AAH18149 (human). Examples of GeneBank Accession Nos. for the nucleotide sequence of various species of GM-CSF include NM_000758.3 (human), NM_009969.4 (Mus musculus), and NM_053852.1 (rattus norvegicus). In a specific embodiment, the GM-CSF is human GM-CSF. As used herein, the terms granulocyte-macrophage colony-stimulating factor” and “GM-CSF” encompass GM-CSF polypeptides that are modified by post-translational processing such as signal peptide cleavage, disulfide bond formation, glycosylation (e.g., N-linked glycosylation), protease cleavage and lipid modification (e.g., S-palmitoylation). In some embodiments, GM-CSF consists of a single polypeptide chain that includes a signal sequence. In other embodiments, GM-CSF consists of a single polypeptide chain that does not include a signal sequence. The signal sequence can be the naturally occurring signal peptide sequence or a variant thereof In some embodiments, the signal peptide is a GM-CSF signal peptide. In some embodiments, the signal peptide is heterologous to a GM-CSF signal peptide.

In a specific embodiment, a transgene encoding a GM-CSF derivative is incorporated into the genome of an APMV described herein. See, e.g., Section 5.1.2.1, supra, for types and strains of APMV that may be used. In a specific embodiment, the transgene encodes a human GM-CSF derivative. One of skill in the art would be able to use such sequence information to produce a transgene for incorporation into the genome of an APMV described herein. In a specific embodiment, a GM-CSF derivative has at least 70%, 75%, 80%, 85%, 90%, 95%, 96%, 98%, or 99% amino acid sequence identity to a GM-CSF known to those of skill in the art. Methods/techniques known in the art may be used to determine sequence identity (see, e.g., “Best Fit” or “Gap” program of the Sequence Analysis Software Package, version 10; Genetics Computer Group, Inc.). In a specific embodiment, a GM-CSF derivative comprises deleted forms of a known GM-CSF, wherein up to about 20, 15, 10, 9, 8, 7, 6, 5, 4, 3, 2 or 1 amino acid residues are deleted from the known GM-CSF (e.g., human GM-CSF). Also provided herein are GM-CSF derivatives comprising deleted forms of a known GM-CSF, wherein about 1-3, 3-5, 5-7, 7-10, 10-15, or 15-20 amino acid residues are deleted from the known GM-CSF (e.g., human GM-CSF). Further provided herein are GM-CSF derivatives comprising altered forms of a known GM-CSF (e.g., human GM-CSF), wherein up to about 20, 15, 10, 9, 8, 7, 6, 5, 4, 3, 2 or 1 amino acid residues of the known GM-CSF are substituted (e.g., conservatively substituted) with other amino acids. In some embodiments, a GM-CSF derivative comprises up to about 20, 15, 10, 9, 8, 7, 6, 5, 4, 3, 2 or 1 conservatively substituted amino acids. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophylic (Cys, Ser, Thr), acidic (Asp, Glu), basic (Asn, Gln, His, Lys, Arg), conformation disruptors (Gly, Pro) and aromatic (Trp, Tyr, Phe).

In a specific embodiment, a GM-CSF derivative is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a native GM-CSF (e.g., human GM-CSF). In another specific embodiment, a GM-CSF derivative is a polypeptide encoded by a nucleic acid sequence that is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a nucleic acid sequence encoding a native GM-CSF (e.g., human GM-CSF). In another specific embodiment, a GM-CSF derivative contains 1, 2, 3, 4, 5, 6, 7, 8, 9, 10, 11, 12, 13, 14, 15, 16, 17, 18, 19, 20 or more, or 2 to 5, 2 to 10, 5 to 10, 5 to 15, 5 to 20, 10 to 15, or 15 to 20 amino acid mutations (i.e., additions, deletions and/or substitutions) relative to a native GM-CSF (e.g., human GM-CSF). In another specific embodiment, a GM-CSF derivative is a polypeptide encoded by nucleic acid sequence that can hybridize under high, moderate or typical stringency hybridization conditions to a nucleic acid sequence encoding a native GM-CSF (e.g., human GM-CSF). Hybridization conditions are known to one of skill in the art (see, e.g., U.S. Patent Application No. 2005/0048549 at, e.g., paragraphs 72 and 73). In another specific embodiment, a GM-CSF derivative is a polypeptide encoded by a nucleic acid sequence that can hybridize under high, moderate or typical stringency hybridization conditions to a nucleic acid sequence encoding a fragment of a native GM-CSF (e.g., human GM-CSF) of at least 10 contiguous amino acids, at least 12 contiguous amino acids, at least 15 contiguous amino acids, at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, at least 150 contiguous amino acids, or 10 to 20, 20 to 50, 25 to 75, 25 to 100, 25 to 150, 50 to 75, 50 to 100, 75 to 100, 50 to 150, 75 to 150, 100 to 150, or 100 to 200 contiguous amino acids. In another specific embodiment, a GM-CSF derivative is a fragment of a native GM-CSF (e.g., human GM-CSF). GM-CSF derivatives also include polypeptides that comprise the amino acid sequence of a naturally occurring mature form of GM-CSF and a heterologous signal peptide amino acid sequence. In addition, GM-CSF derivatives include polypeptides that have been chemically modified by, e.g., glycosylation, acetylation, pegylation, phosphorylation, amidation, derivitization by known protecting/blocking groups, proteolytic cleavage, linkage to a cellular ligand or other protein moiety, etc. Further, GM-CSF derivatives include polypeptides comprising one or more non-classical amino acids. In specific embodiments, the GM-CSF derivative retains one, two, or more, or all of the functions of the native GM-CSF from which it was derived. Examples of functions of GM-CSF include the stimulation granulocytes and macrophages from bone marrow precursor cells to proliferate and the recruitment of circulating neutrophils, monocytes and lymphocytes. Tests for determining whether or not a GM-CSF derivative retains one or more functions of the native GM-CSF from which it was derived are known to one of skill in the art and examples are provided herein.

In specific embodiments, the transgene encoding GM-CSF or a derivative thereof in a packaged genome of a recombinant APMV described herein is codon optimized. In a specific embodiment, the nucleotide sequence(s) encoding one or both subunits of a native GM-CSF may be codon optimized.

### 5.1.2.3 Codon Optimization

Any codon optimization technique known to one of skill in the art may be used to codon optimize a nucleic acid sequence encoding a protein of interest (e.g., IL-2, IL-15Ra-IL-15, GM-CSF, HPV-16 E6, or HPV-16 E7). Methods of codon optimization are known in the art, e.g, the OptimumGene™ (GenScript®) protocol and Genewiz® protocol, which are incorporated by reference herein in its entirety. See also U.S. Pat. No. 8,326,547 for methods for codon optimization, which is incorporated herein by reference in its entirety.

As an exemplary method for codon optimization, each codon in the open frame of the nucleic acid sequence encoding a protein of interest or a domain thereof (e.g., IL-2, IL-15Ra-IL-15, GM-CSF, HPV-16 E6, or HPV-16 E7) is replaced by the codon most frequently used in mammalian proteins. This may be done using a web-based program (www.encorbio.com/protocols/Codon.htm) that uses the Codon Usage Database, maintained by the Department of Plant Gene Research in Kazusa, Japan. This nucleic acid sequence optimized for mammalian expression may be inspected for: (1) the presence of stretches of 5xA or more that may act as transcription terminators; (2) the presence of restriction sites that may interfere with subcloning; and (3) compliance with the rule of six. Following inspection, (1) stretches of 5xA or more that may act as transcription terminators may be replaced by synonymous mutations; (2) restriction sites that may interfere with subcloning may be replaced by synonymous mutations; (3) APMV regulatory signals (gene end, intergenic and gene start sequences), and Kozak sequences for optimal protein expression may be added; and (4) nucleotides may be added in the non-coding region to ensure compliance with the rule of six. Synonymous mutations are typically nucleotide changes that do not change the amino acid encoded. For example, in the case of a stretch of 6 As (AAAAAA), which sequence encodes Lys-Lys, a synonymous sequence would be AAGAAG, which sequence also encodes Lys-Lys.

### 5.2 Construction of APMVs

The APMVs described herein (see, e.g., Sections 5.1, 6 and 7) can be generated using the reverse genetics technique. The reverse genetics technique involves the preparation of synthetic recombinant viral RNAs that contain the non-coding regions of the negative-strand, viral RNA which are essential for the recognition by viral polymerases and for packaging signals necessary to generate a mature virion. The recombinant RNAs are synthesized from a recombinant DNA template and reconstituted in vitro with purified viral polymerase complex to form recombinant ribonucleoproteins (RNPs) which can be used to transfect cells. A more efficient transfection is achieved if the viral polymerase proteins are present during transcription of the synthetic RNAs either in vitro or in vivo. The synthetic recombinant RNPs can be rescued into infectious virus particles. The foregoing techniques are described in U.S. Pat. No. 5,166,057 issued Nov. 24, 1992; in U.S. Pat. No. 5,854,037 issued Dec. 29, 1998; in U.S. Pat. No. 6,146,642 issued Nov. 14, 2000; in European Patent Publication EP 0702085A1, published Feb. 20, 1996; in U.S. patent application Ser. No. 09/152,845; in International Patent Publications PCT WO97/12032 published Apr. 3, 1997; WO96/34625 published Nov. 7, 1996; in European Patent Publication EP A780475; WO 99/02657 published Jan. 21, 1999; WO 98/53078 published Nov. 26, 1998; WO 98/02530 published Jan. 22, 1998; WO 99/15672 published Apr. 1, 1999; WO 98/13501 published Apr. 2, 1998; WO 97/06270 published Feb. 20, 1997; and EPO 780 475A1 published Jun. 25, 1997, each of which is incorporated by reference herein in its entirety.

The helper-free plasmid technology can also be utilized to engineer an APMV described herein. In particular, helper-free plasmid technology can be utilized to engineer a recombinant APMV described herein. Briefly, a complete cDNA of an APMV (e.g., an APMV-4 strain) is constructed, inserted into a plasmid vector and engineered to contain a unique restriction site between two transcription units (e.g., the APMV P and M transcription units; or the APMV HN and L transcription units). A nucleotide sequence encoding a heterologous amino acid sequence (e.g., a transgene or other sequence) may be inserted into the viral genome at the unique restriction site. Alternatively, a nucleotide sequence encoding a heterologous amino acid sequence (e.g., a transgene or other sequence) may be engineered into an APMV transcription unit so long as the insertion does not affect the ability of the virus to infect and replicate. The single segment is positioned between a T7 promoter and the hepatitis delta virus ribozyme to produce an exact negative or positive transcript from the T7 polymerase. The plasmid vector and expression vectors comprising the necessary viral proteins are transfected into cells leading to production of recombinant viral particles (see, e.g., International Publication No. WO 01/04333; U.S. Pat. Nos. 7,442,379, 6,146,642, 6,649,372, 6,544,785 and 7,384,774; Swayne et al. (2003). Avian Dis. 47:1047-1050; and Swayne et al. (2001). J. Virol. 11868-11873, each of which is incorporated by reference in its entirety). See also, e.g., Nolden et al., Scientific Reports 6: 23887 (2016) for reverse genetic techniques to generate negative-strand RNA viruses, which is incorporated herein by reference.

Bicistronic techniques to produce multiple proteins from a single mRNA are known to one of skill in the art. Bicistronic techniques allow the engineering of coding sequences of multiple proteins into a single mRNA through the use of IRES sequences. IRES sequences direct the internal recruitment of ribosomes to the RNA molecule and allow downstream translation in a cap independent manner. Briefly, a coding region of one protein is inserted downstream of the ORF of a second protein. The insertion is flanked by an IRES and any untranslated signal sequences necessary for proper expression and/or function. The insertion must not disrupt the open reading frame, polyadenylation or transcriptional promoters of the second protein (see, e.g., Garcia-Sastre et al., 1994, J. Virol. 68:6254-6261 and Garcia-Sastre et al., 1994 Dev. Biol. Stand. 82:237-246, each of which are incorporated by reference herein in their entirety).

Methods for cloning a recombinant APMV to encode a transgene and express a heterologous protein encoded by the transgene are known to one skilled in the art, such as, e.g., insertion of the transgene into a restriction site that has been engineered into the APMV genome, inclusion an appropriate signals in the transgene for recognition by the APMV RNA-dependent-RNA polymerase (e.g., sequences upstream of the open reading frame of the transgene that allow for the APMV polymerase to recognize the end of the previous gene and the beginning of the transgene, which may be, e.g., spaced by a single nucleotide intergenic sequence), inclusion of a valid Kozak sequence (e.g., to improve eukaryotic ribosomal translation); incorporation of a transgene that satisfies the “rule of six” for APMV cloning; and inclusion of silent mutations to remove extraneous gene end and/or gene start sequences within the transgene. Regarding the Rule of Six, one skilled in the art will understand that efficient replication of APMV (and more generally, most members of the paramyxoviridae family) is dependent on the genome length being a multiple of six, known as the “rule of six” (see, e.g., Calain, P. & Roux, L. The rule of six, a basic feature of efficient replication of Sendai virus defective interfering RNA. J. Virol. 67, 4822-4830 (1993)). Thus, when constructing a recombinant APMV described herein, care should be taken to satisfy the “Rule of Six” for APMV cloning. Methods known to one skilled in the art to satisfy the Rule of Six for APMV cloning may be used, such as, e.g., addition of nucleotides downstream of the transgene. See, e.g., Ayllon et al., Rescue of Recombinant Newcastle Disease Virus from cDNA. J. Vis. Exp. (80), e50830, doi:10.3791/50830 (2013) for a discussion of methods for cloning and rescuing of APMV (e.g., a recombinant APMV), which is incorporated by reference herein in its entirety.

### 5.3 Propagation of APMVs

An APMV described herein (e.g., a naturally occurring APMV or a recombinant APMV; see, also, e.g., Sections 5.1, 6 and 7) can be propagated in any substrate that allows the virus to grow to titers that permit the uses of the viruses described herein. In one embodiment, the substrate allows the APMV described herein (e.g., a naturally occurring APMV or a recombinant APMV; see, also, e.g., Sections 5.1, 6 and 7). In a specific embodiment, the substrate allows the APMV described herein (e.g., a naturally occurring APMV or a recombinant APMV; see, also, e.g., Sections 5.1, 6 and 7) to grow to titers comparable to those determined for the corresponding wild-type viruses.

An APMV described herein (e.g., a naturally occurring APMV or a recombinant APMV; see, also, e.g., Sections 5.1, 6 and 7) may be grown in cells (e.g., avian cells, chicken cells, etc.) that are susceptible to infection by the viruses, embryonated eggs (e.g., chicken eggs or quail eggs) or animals (e.g., birds). Such methods are well-known to those skilled in the art. In a specific embodiment, an APMV described herein (e.g., a naturally occurring APMV or a recombinant APMV; see, also, e.g., Sections 5.1, 6 and 7) may be propagated in cancer cells, e.g., carcinoma cells (e.g., breast cancer cells and prostate cancer cells), sarcoma cells, leukemia cells, lymphoma cells, and germ cell tumor cells (e.g., testicular cancer cells and ovarian cancer cells). In another specific embodiment, an APMV described herein (e.g., a naturally occurring APMV or a recombinant APMV; see, also, e.g., Sections 5.1, 6 and 7) may be propagated in a cell line, e.g., cancer cell lines such as HeLa cells, MCF7 cells, B16-F10 cells, CT26 cells, TC-1 cells, THP-1 cells, U87 cells, DU145 cells, Lncap cells, and T47D cells. In certain embodiments, the cells or cell lines (e.g., cancer cells or cancer cell lines) are obtained and/or derived from a human(s). In another embodiment, an APMV described herein (e.g., a naturally occurring APMV or a recombinant APMV; see, also, e.g., Sections 5.1, 6 and 7) is propagated in chicken cells or embryonated eggs. Representative chicken cells include, but are not limited to, chicken embryo fibroblasts and chicken embryo kidney cells. In a specific embodiment, an APMV described herein (e.g., a naturally occurring APMV or a recombinant APMV; see, also, e.g., Sections 5.1, 6 and 7) is propagated in IFN-deficient cells (e.g., IFN-deficient cell lines). In a specific embodiment, an APMV described herein (e.g., a naturally occurring APMV or a recombinant APMV; see, also, e.g., Sections 5.1, 6 and 7) is propagated in Vero cells. In another specific embodiment, an APMV described herein (e.g., a naturally occurring APMV or a recombinant APMV; see, also, e.g., Sections 5.1, 6 and 7) is propagated in cancer cells in accordance with the methods described in Section 6, infra. In another specific embodiment, an APMV described herein (e.g., a naturally occurring APMV or a recombinant APMV; see, also, e.g., Sections 5.1, 6 and 7) is propagated in chicken eggs or quail eggs. In certain embodiments, an APMV described herein (e.g., a naturally occurring APMV or a recombinant APMV; see, also, e.g., Sections 5.1, 6 and 7) is first propagated in embryonated eggs and then propagated in cells (e.g., a cell line).

An APMV described herein (e.g., a naturally occurring APMV or a recombinant APMV; see, also, e.g., Sections 5.1, 6 and 7) may be propagated in embryonated eggs, e.g., from 6 to 14 days old, 6 to 12 days old, 6 to 10 days old, 6 to 9 days old, 6 to 8 days old, 8 days old, 9 days old, 10 days old, 8 to 10 days old, 12 days old, or 10 to 12 days old. Young or immature embryonated eggs can be used to propagate an APMV described herein (e.g., a naturally occurring APMV or a recombinant APMV; see, also, e.g., Sections 5.1, 6 and 7). Immature embryonated eggs encompass eggs which are less than ten day old eggs, e.g., eggs 6 to 9 days old or 6 to 8 days old that are IFN-deficient. Immature embryonated eggs also encompass eggs which artificially mimic immature eggs up to, but less than ten day old, as a result of alterations to the growth conditions, e.g., changes in incubation temperatures; treating with drugs; or any other alteration which results in an egg with a retarded development, such that the IFN system is not fully developed as compared with ten to twelve day old eggs. In a specific embodiment, an APMV described herein (e.g., a naturally occurring APMV or a recombinant APMV; see, also, e.g., Sections 5.1, 6 and 7) are propagated in 8 or 9 day old embryonated chicken eggs. In another specific embodiment, an APMV described herein (e.g., a naturally occurring APMV or a recombinant APMV; see, also, e.g., Sections 5.1, 6 and 7) are propagated in 10 day old embryonated chicken eggs. An APMV described herein (e.g., a naturally occurring APMV or a recombinant APMV; see, also, e.g., Sections 5.1, 6 and 7) can be propagated in different locations of the embryonated egg, e.g., the allantoic cavity. For a detailed discussion on the growth and propagation viruses, see, e.g., U.S. Pat. No. 6,852,522 and U.S. Pat. No. 7,494,808, both of which are hereby incorporated by reference in their entireties.

In a specific embodiment, provided herein is a cell (e.g., a cell line) or embryonated egg (e.g., a chicken embryonated egg) comprising an APMV described herein (e.g., a naturally occurring APMV or a recombinant APMV; see, also, e.g., Sections 5.1, 6 and 7). Examples of cells as well as embryonated eggs which may comprise an APMV described herein may be found above. In a specific embodiment, provided herein is a method for propagating an APMV described herein (e.g., a naturally occurring APMV or a recombinant APMV; see, also, e.g., Sections 5.1, 6 and 7), the method comprising culturing a substrate (e.g., a cell line or embryonated egg) infected with the APMV. In another specific embodiment, provided herein is a method for propagating an APMV described herein (e.g., a naturally occurring APMV or a recombinant APMV; see, also, e.g., Sections 5.1, 6 and 7), the method comprising: (a) culturing a substrate (e.g., a cell line or embryonated egg) infected with the APMV; and (b) isolating or purifying the APMV from the substrate. In certain embodiments, these methods involve infecting the substrate with the APMV prior to culturing the substrate. See, e.g., Section 6, infra, for methods that may be used to propagate an APMV described herein (e.g., a naturally occurring APMV or a recombinant APMV described herein).

For virus isolation, an APMV described herein (e.g., a naturally occurring APMV or a recombinant APMV; see, also, e.g., Sections 5.1, 6 and 7) can be removed from embryonated eggs or cell culture and separated from cellular components, typically by well known clarification procedures, e.g., such as centrifugation, depth filtration, and microfiltration, and may be further purified as desired using procedures well known to those skilled in the art, e.g., tangential flow filtration (TFF), density gradient centrifugation, differential extraction, or chromatography.

In a specific embodiment, provided herein is a method for producing a pharmaceutical composition (e.g., an immunogenic composition) comprising an APMV described herein (e.g., a naturally occurring APMV or a recombinant APMV; see, also, e.g., Sections 5.1 and 6), the method comprising (a) propagating an APMV described herein (e.g., a naturally occurring APMV or a recombinant APMV; see, also, e.g., Sections 5.1, 6 and 7) in a cell (e.g., a cell line) or embyronated egg; and (b) isolating the APMV from the cell or embyronated egg. The method may further comprise adding the APMV to a container along with a pharmaceutically acceptable carrier.

In a specific embodiment, an APMV described herein (e.g., a naturally occurring APMV or a recombinant APMV; see, also, e.g., Sections 5.1, 6 and 7) is propagated, isolated, and/or purified according to a method described in Section 6. In a specific embodiment, an APMV described herein (e.g., a naturally occurring APMV or a recombinant APMV; see, also, e.g., Sections 5.1, 6 and 7) is either propagated, isolated, or purified, or any two or all of the foregoing, using a method described in Section 6.

### 5.4 Compositions and Routes of Administration

Encompassed herein is the use of an APMV described herein (e.g., a naturally occurring APMV or a recombinant APMV described herein) in compositions. In a specific embodiment, the compositions are pharmaceutical compositions. The compositions may be used in methods of treating cancer.

In one embodiment, a pharmaceutical composition comprises an APMV described herein (e.g., a naturally occurring APMV or a recombinant APMV described herein), in an admixture with a pharmaceutically acceptable carrier. In a specific embodiment, the APMV is an APMV-4 described herein. In other embodiments, the APMV is an APMV-6, APMV-7, APMV-8 or APMV-9 described herein. In a specific embodiment, the APMV is a recombinant APMV described herein. In a particular embodiment, the APMV is a recombinant APMV-4 comprising a packaged genome, wherein the packaged genome comprises the negative sense RNA transcribed from the cDNA sequence set forth in SEQ ID NO: 14. In some embodiments, the pharmaceutical composition further comprises one or more additional prophylactic or therapeutic agents, such as described in Section 5.5.2, infra. In a specific embodiment, a pharmaceutical composition comprises an effective amount of an APMV described herein (e.g., a naturally occurring APMV or a recombinant APMV described herein), and optionally one or more additional prophylactic or therapeutic agents, in a pharmaceutically acceptable carrier. In some embodiments, an APMV described herein (e.g., a naturally occurring APMV or a recombinant APMV described herein) is the only active ingredient included in the pharmaceutical composition.

In another embodiment, a pharmaceutical composition (e.g., an oncolysate vaccine) comprises a protein concentrate or a preparation of plasma membrane fragments from APMV infected cancer cells, in an admixture with a pharmaceutically acceptable carrier. In some embodiments, the pharmaceutical composition further comprises one or more additional prophylactic or therapeutic agents, such as described in Section 5.5.2, infra.. In another embodiment, a pharmaceutical composition (e.g., a whole cell vaccine) comprises cancer cells infected with APMV, in an admixture with a pharmaceutically acceptable carrier. In some embodiments, the pharmaceutical composition further comprises one or more additional prophylactic or therapeutic agents, such as described in Section 5.5.2, infra.

The pharmaceutical compositions provided herein can be in any form that allows for the composition to be administered to a subject. In a specific embodiment, the pharmaceutical compositions are suitable for veterinary administration, human administration or both. As used herein, the term “pharmaceutically acceptable” means approved by a regulatory agency of the Federal or a state government or listed in the U.S. Pharmacopeia or other generally recognized pharmacopeias for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. The formulation should suit the mode of administration.

In a specific embodiment, the pharmaceutical compositions are formulated to be suitable for the intended route of administration to a subject. The pharmaceutical composition may be formulated for systemic or local administration to a subject. For example, the pharmaceutical composition may be formulated to be suitable for parenteral, intravenous, intraarterial, intrapleural, inhalation, intraperitoneal, oral, intradermal, colorectal, intraperitoneal, intracranial, and intratumoral administration. In a specific embodiment, the pharmaceutical composition may be formulated for intravenous, intraarterial, oral, intraperitoneal, intranasal, intratracheal, intrapleural, intracranial, subcutaneous, intramuscular, topical, pulmonary, or intratumoral administration.

In a specific embodiment, a pharmaceutical composition comprising an APMV described herein (e.g., a naturally occurring APMV or a recombinant APMV described herein) is formulated to be suitable for intratumoral administration to the subject (e.g., human subject). In a specific embodiment, a pharmaceutical composition comprising an APMV-4 described herein is formulated for intratumoral administration to a subject (e.g., a human subject). In other specific embodiments, a pharmaceutical composition comprising an APMV-6, APMV-7, APMV-8 or APMV-9 described herein is formulated for intratumoral administration to a subject (e.g., a human subject). In another specific embodiment, a pharmaceutical composition comprising a recombinant APMV described herein is formulated for intratumoral administration to the subject (e.g., human subject).

In a specific embodiment, a pharmaceutical composition comprising an APMV described herein (e.g., a naturally occurring APMV or a recombinant APMV described herein) is formulated to be suitable for intravenous administration to the subject (e.g., human subject). In a specific embodiment, a pharmaceutical composition comprising an APMV-4 described herein is formulated for intravenous administration to a subject (e.g., a human subject). In other specific embodiments, a pharmaceutical composition comprising an APMV-6, APMV-7, APMV-8 or APMV-9 described herein is formulated for intravenous administration to a subject (e.g., a human subject). In another specific embodiment, a pharmaceutical composition comprising a recombinant APMV described herein is formulated for intravenous administration to the subject (e.g., human subject).

To the extent an APMV described herein (e.g., a naturally occurring APMV or recombinant APMV described herein) is administered in combination with another therapy, the other therapy (e.g., prophylactic or therapeutic agent) may be administered in a separate pharmaceutical composition. In other words, two separate pharmaceutical compositions may be administered to a subject to treat cancer—one pharmaceutical composition comprising an APMV described herein (e.g., a naturally occurring APMV or recombinant APMV described herein) in an admixture with a pharmaceutically acceptable carrier, and a second pharmaceutical composition comprising another therapy (such as, e.g., described in Section 5.5.2, infra) in an admixture with a pharmaceutically acceptable carrier. The two pharmaceutical composition may be formulated for the same route of administration to the subject (e.g., human subject) or different routes of administration to the subject (e.g., human subject). For example, the pharmaceutical composition comprising an APMV described herein may be formulated for local administration to a tumor of a subject (e.g. a human subject), while the pharmaceutical composition comprising another therapy (such as, e.g., described in Section 5.5.2, infra) is formulated for systemic administration to the subject (e.g., human subject). In one specific example, the pharmaceutical composition comprising an APMV described herein may be formulated for intratumoral administration to the subject (e.g., human subject), while the pharmaceutical composition comprising another therapy (such as, e.g., described in Section 5.5.2, infra) is formulated for intravenous administration, subcutaneous administration or another route of administration to the subject (e.g., human subject). In another example, the pharmaceutical composition comprising an APMV described herein and the pharmaceutical composition comprising another therapy (such as, e.g., described in Section 5.5.2, infra) may both be formulated for intravenous administration to the subject (e.g., human subject). In certain embodiments, a pharmaceutical composition comprising a therapy, such as, e.g., described in Section 5.5.2, infra, which is used in combination with an APMV described herein or a composition thereof, is formulated for administration by an approved route, such as described in the Physicans' Desk Reference 71s^{t} ed (2017).

### 5.5 Uses of APMV

In one aspect, an APMV described herein (e.g., a naturally occurring or recombinant APMV described herein) or a composition thereof, an oncolysate described herein or a composition thereof, or whole cell vaccine may be used in the treatment of cancer. In one embodiment, provided herein are methods for treating cancer, comprising administering to a subject in need thereof an APMV described herein (e.g., a naturally occurring or recombinant APMV described herein) or a composition thereof. In a specific embodiment, provided herein is a method for treating cancer, comprising administering to a subject in need thereof an effective amount of an APMV described herein (e.g., a naturally occurring or recombinant APMV described herein) or a composition thereof. In another embodiment, an oncolysate or whole cell vaccine described herein may be used to treat cancer as described herein. See Section 5.5.4 for the types of cancer that may be treated in accordance with the methods described herein, Section 5.5.3 for the types of patients that may be treated in accordance with the methods described herein, and Section 5.5.1 for exemplary dosages and regimens for treating cancer in accordance with the methods described herein.

In certain embodiments, an APMV described herein (e.g., a naturally occurring or recombinant APMV described herein) or a composition thereof is the only active ingredient administered to treat cancer. In specific embodiments, an APMV described herein (e.g., a naturally occurring or recombinant APMV described herein) is the only active ingredient in a composition administered to treat cancer.

An APMV described herein (e.g., a naturally occurring or recombinant APMV described herein) or a composition thereof may be administered locally or systemically to a subject. For example, an APMV described herein (e.g., a naturally occurring or recombinant APMV described herein) or a composition thereof may be administered parenterally (e.g., intraperitoneally, intravenously, intra-arterially, intradermally, intramuscularly, or subcutaneously), intratumorally, intra-nodally, intrapleurally, intranasally, intracavitary, intracranially, orally, rectally, by inhalation, or topically to a subject. In a specific embodiment, an APMV described herein (e.g., a naturally occurring or recombinant APMV described herein) or a composition thereof is administered intratumorally. Image-guidance may be used to administer an APMV described herein (e.g., a naturally occurring or recombinant APMV described herein) or a composition thereof to the subject. In a specific embodiment, an APMV described herein (e.g., a naturally occurring or recombinant APMV described herein) or a composition thereof is administered intravenously.

In certain embodiments, the methods described herein include the treatment of cancer for which no treatment is available. In some embodiments, an APMV described herein (e.g., a naturally occurring or recombinant APMV described herein) or a composition thereof is administered to a subject to treat cancer as an alternative to other conventional therapies.

In one embodiment, provided herein is a method for treating cancer, comprising administering to a subject in need thereof an APMV described herein (e.g., a naturally occurring or recombinant APMV described herein) or a composition thereof and one or more additional therapies, such as described in Section 5.5.2, infra. In a specific embodiment, provided herein is a method for treating cancer, comprising administering to a subject in need thereof an effective amount of an APMV described herein (e.g., a naturally occurring or recombinant APMV described herein) or a composition thereof and an effective amount of one or more additional therapies, such as described in Section 5.5.2, infra. In a particular embodiment, one or more therapies are administered to a subject in combination with an APMV described herein (e.g., a naturally occurring or recombinant APMV described herein) or a composition thereof to treat cancer. In a specific embodiment, the additional therapies are currently being used, have been used or are known to be useful in treating cancer. In another embodiment, a recombinant APMV described herein (e.g., a recombinant APMV described in Section 5.1, supra, or Section 7) or a composition thereof is administered to a subject in combination with a supportive therapy, a pain relief therapy, or other therapy that does not have a therapeutic effect on cancer. In certain embodiments, an APMV described herein (e.g., a naturally occurring or recombinant APMV described herein) and one or more additional therapies are administered in the same composition. In other embodiments, an APMV described herein (e.g., a naturally occurring or recombinant APMV described herein) and one or more additional therapies are administered in different compositions. An APMV described herein (e.g., a naturally occurring or recombinant APMV described herein) or a composition thereof in combination with one or more additional therapies, such as described herein in Section 5.5.2, infra, may be used as any line of therapy (e.g., a first, second, third, fourth or fifth line therapy) for treating cancer in accordance with a method described herein.

In certain embodiments, two, three or multiple APMVs (including one, two or more recombinant APMVs described herein) are administered to a subject to treat cancer.

In a specific embodiment, a method of treating cancer described herein may result in a beneficial effect for a subject, such as the reduction, decrease, attenuation, diminishment, stabilization, remission, suppression, inhibition or arrest of the development or progression of cancer, or a symptom thereof. In certain embodiments, a method of treating cancer described herein results in at least one, two or more of the following effects: (i) the reduction or amelioration of the severity of cancer and/or a symptom associated therewith; (ii) the reduction in the duration of a symptom associated with cancer; (iii) the prevention in the recurrence of a symptom associated with cancer; (iv) the regression of cancer and/or a symptom associated therewith; (v) the reduction in hospitalization of a subject; (vi) the reduction in hospitalization length; (vii) the increase in the survival of a subject; (viii) the inhibition of the progression of cancer and/or a symptom associated therewith; (ix) the enhancement or improvement of the therapeutic effect of another therapy; (x) a reduction or elimination in the cancer cell population; (xi) a reduction in the growth of a tumor or neoplasm; (xii) a decrease in tumor size; (xiii) a reduction in the formation of a tumor; (xiv) eradication, removal, or control of primary, regional and/or metastatic cancer; (xv) a decrease in the number or size of metastases; (xvi) a reduction in mortality; (xvii) an increase in cancer-free survival rate of patients; (xviii) an increase in relapse-free survival; (xix) an increase in the number of patients in remission; (xx) a decrease in hospitalization rate; (xxi) the size of the tumor is maintained and does not increase in size or increases the size of the tumor by less than 5% or 10% after administration of a therapy as measured by conventional methods available to one of skill in the art, such as MM, X-ray, CT Scan and PET scan; (xxii) the prevention of the development or onset of cancer and/or a symptom associated therewith; (xxiii) an increase in the length of remission in patients; (xxiv) the reduction in the number of symptoms associated with cancer; (xxv) an increase in symptom-free survival of cancer patients; (xxvi) limitation of or reduction in metastasis; (xxvii) overall survival; (xxviii) progression-free survival (as assessed, e.g., by RECIST v1.1.); (xxix) overall response rate; and/or (xxx) an increase in response duration. In some embodiments, the treatment/therapy that a subject receives does not cure cancer, but prevents the progression or worsening of the disease. In certain embodiments, a method of treating cancer described herein does not prevent the onset/development of cancer, but may prevent the onset of cancer symptoms. Any method known to the skilled artisan may be utilized to evaluate the treatment/therapy that a subject receives. In a specific embodiment, the efficacy of a treatment/therapy is evaluated according to the Response Evaluation Criteria In Solid Tumors (“RECIST”) published rules. In a specific embodiment, the efficacy of a treatment/therapy is evaluated according to the RECIST rules published in February 2000 (also referred to as “RECIST 1”) (see, e.g., Therasse et al., 2000, Journal of National Cancer Institute, 92(3):205-216, which is incorporated by reference herein in its entirety). In a specific embodiment, the efficacy of a treatment/therapy is evaluated according to the RECIST rules published in January 2009 (also referred to as “RECIST 1.1”) (see, e.g., Eisenhauer et al., 2009, European Journal of Cancer, 45:228-247, which is incorporated by reference herein in its entirety). In a specific embodiment, the efficacy of a treatment/therapy is evaluated according to the RECIST rules utilized by the skilled artisan at the time of the evaluation. In a specific embodiment, the efficacy is evaluated according to the immune related RECIST (“irRECIST”) published rules (see, e.g., Bohnsack et al., 2014, ESMO Abstract 4958, which is incorporated by reference herein in its entirety). In a specific embodiment, the efficacy treatment/therapy is evaluated according to the irRECIST rules utilized by the skilled artisan at the time of the evaluation. In a specific embodiment, the efficacy is evaluated through a reduction in tumor-associated serum markers.

### 5.5.1 Dosage and Frequency

The amount of an APMV described herein (e.g., a naturally occurring or recombinant APMV described herein) or a composition thereof which will be effective in the treatment of cancer will depend on the nature of the cancer, the route of administration, the general health of the subject, etc. and should be decided according to the judgment of a medical practitioner. Standard clinical techniques, such as in vitro assays, may optionally be employed to help identify dosage ranges. However, suitable dosage ranges of an APMV described herein (e.g., a naturally occurring or recombinant described herein) for administration are generally about 10², 5×10², 10³, 5×10³, 10⁴, 5×10⁴, 10⁵, 5×10⁵, 10⁶, 5×10⁶, 10⁷, 5×10⁷, 10⁸, 5×10⁸, 1×10⁹, 5×10⁹, 1×10¹⁰, 5×10¹⁰, 1×10¹¹, 5×10¹¹ or 10¹² pfu, and most preferably about 10⁴ to about 10¹², 10⁶ to 10¹², 10⁸ to 10¹², 10⁹ to 10¹² or 10⁹ to 10¹¹ pfu, and can be administered to a subject once, twice, three, four or more times with intervals as often as needed. Dosage ranges of oncolysate vaccines for administration may include 0.001 mg, 0.005 mg, 0.01 mg, 0.05 mg. 0.1 mg. 0.5 mg, 1.0 mg, 2.0 mg. 3.0 mg, 4.0 mg, 5.0 mg, 10.0 mg, 0.001 mg to 10.0 mg, 0.01 mg to 1.0 mg, 0.1 mg to 1 mg, and 0.1 mg to 5.0 mg, and can be administered to a subject once, twice, three or more times with intervals as often as needed. Dosage ranges of whole cell vaccines for administration may include 10², 5×10², 10³, 5×10³, 10⁴, 5×10⁴, 10⁵, 5×10⁵, 10⁶, 5×10⁶, 10⁷, 5×10⁷, 10⁸, 5×10⁸, 1×10⁹, 5×10⁹, 1×10¹⁰, 5×10¹⁰, 1×10¹¹, 5×10¹¹ or 10¹² cells, and can be administered to a subject once, twice, three or more times with intervals as often as needed. In certain embodiments, a dosage(s) of an APMV described herein similar to a dosage(s) currently being used in clinical trials for NDV is administered to a subject.

In certain embodiments, an APMV described herein (e.g., a naturally occurring or recombinant described herein) or a composition thereof is administered to a subject as a single dose followed by a second dose 1 to 6 weeks, 1 to 5 weeks, 1 to 4 weeks, 1 to 3 weeks, 1 to 2 weeks later. In accordance with these embodiments, booster inoculations may be administered to the subject at 3 to 6 month or 6 to 12 month intervals following the second inoculation.

In certain embodiments, an APMV described herein (e.g., a naturally occurring or recombinant described herein) or composition thereof is administered to a subject in combination with one or more additional therapies, such as a therapy described in Section 5.5.2, infra. The dosage of the other one or more additional therapies will depend upon various factors including, e.g., the therapy, the nature of the cancer, the route of administration, the general health of the subject, etc. and should be decided according to the judgment of a medical practitioner. In specific embodiments, the dose of the other therapy is the dose and/or frequency of administration of the therapy recommended for the therapy for use as a single agent is used in accordance with the methods disclosed herein. In other embodiments, the dose of the other therapy is a lower dose and/or involves less frequent administration of the therapy than recommended for the therapy for use as a single agent is used in accordance with the methods disclosed herein. Recommended doses for approved therapies can be found in the Physicians' Desk Reference (e.g., the 71^{st} ed. of the Physicians' Desk Reference (2017)).

In certain embodiments, an APMV described herein (e.g., a naturally occurring or recombinant APMV described herein) or composition thereof is administered to a subject concurrently with the administration of one or more additional therapies. In other embodiments, an APMV described (e.g., a naturally occurring or recombinant APMV described herein) or composition thereof is administered to a subject every 3 to 7 days, 1 to 6 weeks, 1 to 5 weeks, 1 to 4 weeks, 2 to 4 weeks, 1 to 3 weeks, or 1 to 2 weeks and one or more additional therapies (such as described in Section 5.5.2, infra) is administered every 3 to 7 days, 1 to 6 weeks, 1 to 5 weeks, 1 to 4 weeks, 1 to 3 weeks, or 1 to 2 weeks.

### 5.5.2 Additional Therapies

Additional therapies that can be used in a combination with an APMV described herein (e.g., a naturally occurring or recombinant APMV described herein) or a composition thereof for the treatment of cancer include, but are not limited to, small molecules, synthetic drugs, peptides (including cyclic peptides), polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules. In a specific embodiment, the additional therapy is a chemotherapeutic agent. In a specific embodiment, an additional therapy described herein may be used in combination with an oncolysate or whole cell vaccine described herein.

In some embodiments, an APMV described herein (e.g., a naturally occurring or recombinant APMV described herein) or a composition thereof is used in combination with radiation therapy comprising the use of x-rays, gamma rays and other sources of radiation to destroy cancer cells. In specific embodiments, the radiation therapy is administered as external beam radiation or teletherapy, wherein the radiation is directed from a remote source. In other embodiments, the radiation therapy is administered as internal therapy or brachytherapy wherein a radioactive source is placed inside the body close to cancer cells and/or a tumor mass.

Specific examples of anti-cancer agents that may be used in combination with an APMV described herein or a composition thereof include: hormonal agents (e.g., aromatase inhibitor, selective estrogen receptor modulator (SERM), and estrogen receptor antagonist), chemotherapeutic agents (e.g., microtubule disassembly blocker, antimetabolite, topoisomerase inhibitor, and DNA crosslinker or damaging agent), anti-angiogenic agents (e.g., VEGF antagonist, receptor antagonist, integrin antagonist, vascular targeting agent (VTA)/vascular disrupting agent (VDA)), radiation therapy, and conventional surgery.

In particular embodiments, an APMV described herein (e.g., a naturally occurring or recombinant APMV described herein) or a composition thereof is used in combination with an immunomodulatory agent. In a specific embodiment, an APMV described herein (e.g., a naturally occurring APMV or a recombinant APMV described herein) or composition thereof is used in combination with an agonist of a co-stimulatory receptor found on immune cells, such as, e.g., T-lymphocytes (e.g., CD4+ or CD8+ T-lymphocytes), NK cells and/or antigen-presenting cells (e.g., dendritic cells or macrophages), or a composition thereof. Specific examples of co-stimulatory receptors include glucocorticoid-induced tumor necrosis factor receptor (GITR), Inducible T-cell costimulator (ICOS or CD278), OX40 (CD134), CD27, CD28, 4-1BB (CD137), CD40, lymphotoxin alpha (LT alpha), LIGHT (lymphotoxin-like, exhibits inducible expression, and competes with herpes simplex virus glycoprotein D for HVEM, a receptor expressed by T lymphocytes), CD226, cytotoxic and regulatory T cell molecule (CRTAM), death receptor 3 (DR3), lymphotoxin-beta receptor (LTBR), transmembrane activator and CAML interactor (TACI), B cell-activating factor receptor (BAFFR), and B cell maturation protein (BCMA). In a specific embodiment, the agonist of the co-stimulatory molecule binds to a receptor on a cell (e.g., GITR, ICOS, OX40, CD70, 4-1BB, CD40, LIGHT, etc.) and triggers or enhances one or more signal transduction pathways. In a particular embodiment, the agonist of the co-stimulatory receptor is an antibody or ligand that binds to the co-stimulatory receptor and induces or enhances one or more signal transduction pathways. In certain embodiments, the agonist facilitates the interaction between a co-stimulatory receptor and its ligand(s). In certain embodiments, the agonist of a co-stimulatory receptor is an antibody (e.g., monoclonal antibody) that binds to glucocorticoid-induced tumor necrosis factor receptor (GITR), Inducible T-cell costimulator (ICOS or CD278), OX40 (CD134), CD27, CD28, 4-1BB (CD137), CD40, lymphotoxin alpha (LT alpha), LIGHT (lymphotoxin-like, exhibits inducible expression, and competes with herpes simplex virus glycoprotein D for HVEM, a receptor expressed by T lymphocytes), CD226, cytotoxic and regulatory T cell molecule (CRTAM), death receptor 3 (DR3), lymphotoxin-beta receptor (LTBR), transmembrane activator and CAML interactor (TACI), B cell-activating factor receptor (BAFFR), or B cell maturation protein (BCMA). In a specific embodiment, the agonist of a co-stimulatory receptor is an antibody (e.g., monoclonal antibody) that binds to 4-1BB or OX40.

In a specific embodiment, an APMV described herein (e.g., a naturally occurring or recombinant APMV described herein) or a composition thereof is used in combination with an antagonist of an inhibitory receptor found on immune cells, such as, e.g., T-lymphocytes (e.g., CD4+ or CD8+ T-lymphocytes), NK cells and/or antigen-presenting cells (e.g., dendritic cells or macrophages), or a composition thereof. Specific examples of inhibitory receptors include cytotoxic T-lymphocyte-associated antigen 4 (CTLA-4 or CD52), programmed cell death protein 1 (PD-1 or CD279), B and T-lymphocyte attenuator (BTLA), killer cell immunoglobulin-like receptor (KIR), lymphocyte activation gene 3 (LAG3), T-cell membrane protein 3 (TIM3), CD160, adenosine A2a receptor (A2aR), T cell immunoreceptor with immunoglobulin and ITIM domains (TIGIT), leukocyte-associated immunoglobulin-like receptor 1 (LAIR1), and CD160. In a specific embodiment, the antagonist inhibits the action of the inhibitory receptor without provoking a biological response itself. In a specific embodiment, the antagonist is an antibody or ligand that binds to an inhibitor receptor on an immune cell and blocks or dampens binding of the receptor to one or more of its ligands. In a particular embodiment, the antagonist of an inhibitory receptor is an antibody or a soluble receptor that specifically binds to the ligand for the inhibitory receptor and blocks the ligand from binding to the inhibitory receptor and transducing an inhibitory signal(s). Specific examples of ligands for inhibitory receptors include PD-L1, PD-L2, B7-H3, B7-H4, HVEM, Gal9 and adenosine. Specific examples of inhibitory receptors include CTLA-4, PD-1, BTLA, KIR, LAG3, TIM3, and A2aR.

In specific embodiments, the antagonist of an inhibitory receptor is a soluble receptor that specifically binds to a ligand for the inhibitory receptor and blocks the ligand from binding to the inhibitory receptor and transducing an inhibitory signal(s). In certain embodiments, the soluble receptor is a fragment of an inhibitory receptor (e.g., the extracellular domain of an inhibitory receptor). In some embodiments, the soluble receptor is a fusion protein comprising at least a portion of the inhibitory receptor (e.g., the extracellular domain of the native inhibitory receptor), and a heterologous amino acid sequence. In specific embodiments, the fusion protein comprises at least a portion of the inhibitory receptor, and the Fc portion of an immunoglobulin or a fragment thereof In a specific embodiment, the antagonist of an inhibitory receptor is a LAG3-Ig fusion protein (e.g., IMP321).

In another embodiment, the antagonist of an inhibitory receptor is an antibody that specifically binds to a ligand(s) of the inhibitory receptor and blocks the ligand(s) from binding to the inhibitory receptor and transducing an inhibitory signal(s). Specific examples of ligands for inhibitory receptors include PD-L1, PD-L2, B7-H3, B7-H4, HVEM, Gal9 and adenosine. Specific examples of inhibitory receptors include CTLA-4, PD-1, BTLA, KIR, LAG3, TIM3, and A2aR. In a specific embodiment, the antagonist is an antibody that binds to PD-L1 or PD-L2.

In another embodiment, the antagonist of an inhibitory receptor is an antibody that binds to the inhibitory receptor and blocks the binding of the inhibitory receptor to one, two or more of its ligands. In a specific embodiment, the binding of the antibody to the inhibitory receptor does not transduce an inhibitory signal(s) or blocks an inhibitory signal(s). Specific examples of inhibitory receptors include CTLA-4, PD-1, BTLA, KIR, LAG3, TIM3, and A2aR. A specific example of an antibody to inhibitory receptor is anti-CTLA-4 antibody (Leach D R, et al. Science 1996; 271: 1734-1736). In a specific embodiment, an antagonist of an inhibitory receptor is an antagonist of CTLA-4, such as, e.g., Ipilimumab or Tremelimumab.

In certain embodiments, the antagonist of an inhibitory receptor is an antagonist of PD-1, such as, e.g., Nivolumab (MDX-1106 or BMS-936558), pembrolizumab (MK3475), pidlizumab (CT-011), AMP-224 (a PD-L2 fusion protein), Atezoliuzumab (MPDL3280A; anti-PD-L1 monoclonal antibody), Avelumab (an anti-PD-L1 monoclonal antibody) or MDX-1105 (an anti-PD-L1 monoclonal antibody). In certain embodiments, an antagonist of an inhibitory receptor is an antagonist of LAG3, such as, e.g., IMP321.

In a specific embodiment, an antagonist of an inhibitory receptor is an anti-PD-1 antibody that blocks the interaction between PD-1 and its ligands (PD-L1 and PD-L2). Non-limiting examples of antibodies that bind to PD-1 include pembrolizumab (“KEYTRUDA®”; see, e.g., Hamid et al., N Engl J Med. 2013;369:134-44 and Full Prescribing Information for KEYTRUDA, Reference ID: 3862712), nivolumab (“OPDIVO®”; see, e.g., Topalian et al., N Engl J Med. 2012; 366:2443-54 and Full Prescribing Information for OPDIVO (nivolumab), Reference ID: 3677021), and MEDI0680 (also referred to as “AMP-514”; see, e.g., Hamid et al., Ann Oncol. 2016; 27(suppl_6):1050PD). In a specific embodiment, the antagonist of an inhibitory receptor is an anti-PD1 antibody (e.g., pembrolizumab).

In a specific embodiment, an APMV described herein (e.g., a naturally occurring or recombinant APMV described herein) or a composition thereof is used in combination with a checkpoint inhibitor. In a specific embodiment, the checkpoint inhibitor may be an antibody that binds to an inhibitory receptor found on a T cell, such as PD-1, CTLA-4, LAG-3, or TIM-3. In another specific embodiment, the checkpoint inhibitor may be an antibody that binds to an inhibitory receptor found on a T cell, such as PD-1, CTLA-4, LAG-3, or TIM-3 and blocks binding of the inhibitory receptor to its ligand(s).

In a specific embodiment, an APMV described herein (e.g., a naturally occurring or recombinant APMV described herein) or a composition thereof is used in combination with an anti-PD1 antibody that blocks binding of PD1 to its ligand(s) (e.g., either PD-L1, PD-L2, or both), such as described herein or known to one of skill in the art, or a composition thereof In a specific embodiment, the antibody is a monoclonal antibody.

In a specific embodiment, an APMV described herein (e.g., a naturally occurring or recombinant APMV described herein) or a composition thereof is used in combination with an anti-PD-L1 antibody (e.g., an anti-PD-L1 antibody described herein or known to one of skill in art), or a composition thereof. In a specific embodiment, the antibody is a monoclonal antibody.

In a specific embodiment, an APMV described herein (e.g., a naturally occurring or recombinant APMV described herein) or a composition thereof is used in combination with an anti-PD-L2 antibody (e.g., an anti-PD-L2 antibody described herein or known to one of skill in art), or a composition thereof. In a specific embodiment, the antibody is a monoclonal antibody.

In a specific embodiment, an APMV described herein (e.g., a naturally occurring or recombinant APMV described herein) or a composition thereof is used in combination with a RIG-1 agonist (e.g., poly-dA-dT (otherwise known as poly(deoxyadenylic-deoxythymidylic) acid sodium salt)), or a composition thereof. In another specific embodiment, an APMV described herein (e.g., a naturally occurring or recombinant APMV described herein) or a composition thereof is used in combination with an MDA-5 agonist or a composition thereof. In another specific embodiment, an APMV described herein (e.g., a naturally occurring or recombinant APMV described herein) or a composition thereof is used in combination with a NOD 1/NOD2 agonist (e.g., MurNAc-L-Ala-γ-D-Glu-mDAP) or a composition thereof.

In a specific embodiment, an APMV described herein (e.g., a naturally occurring or recombinant APMV described herein) or a composition thereof is used in combination with a chemotherapeutic agent or a composition thereof. In some embodiments, an APMV described herein (e.g., a naturally occurring or recombinant APMV described herein) or a composition thereof is used in combination with an anti-tumor agent(s), alkylating agent(s), antimetabolite(s), plant-derived anti-tumor agent(s), hormonal therapy agent(s), topoisomerase inhibitor(s), camptothecin derivative(s), kinase inhibitor(s), targeted drug(s), antibody(ies), interferon(s) or biological response modifier, or a combination of one or more of the foregoing. Alkylating agents include, e.g., nitrogen mustard N-oxide, cyclophophamide, ifosfamide, thiotepa, ranimustine, nimustine, temozolomide, altretamine, apaziquone, brostallicin, bendamustine, carmustine, estramustine, fotemustine, glufosfamide, ifosfamide, mafosfamide, bendamustin and mitolactol; and platinum-coordinated alkylating compounds, such as, e.g., cisplatin, carboplatin, eptaplatin, lobaplatin, nedaplatin, oxaliplatin or satrplatin. Antimetabolites include, e.g., methotrexate, 6-mercaptopurine riboside, mercaptopurine, 5-fluorouracil, leucovorin, tegafur, doxifluridine, carmofur, cytarabine, cytarabine ocfosfate, enocitabine, gemcitabine, fludarabin, 5-azacitidine, capecitabine, cladribine, clofarabine, decitabine, eflornithine, ethynylcytidine, cytosine arabinoside, hydroxyurea, melphalan, nelarabine, nolatrexed, ocfosfite, disodium premetrexed, pentostatin, pelitrexol, raltitrexed, triapine, trimetrexate, vidarabine, vincristine, and vinorelbine. Hormonal therapy agents include, e.g., exemestane, Lupron, anastrozole, doxercalciferol, fadrozole, formestane, 11 Beta-Hydroxysteroid Dehydrogenase 1 inhibitors, 17-Alpha Hydroxylase/17,20 Lyase Inhibitors such as abiraterone acetate, 5-Alpha Reductase Inhibitors such as Bearfina (finasteride) and Epristeride, anti-estrogens such as tamoxifen citrate and fulvestrant, Trelstar, toremifene, raloxifene, lasofoxifene, letrozole, or anti-androgens such as bicalutamide, flutamide, mifepristone, nilutamide, Casodex, or anti-progesterones and combinations thereof.

Plant-derived anti-tumor substances include, for example, those selected from mitotic inhibitors, for example epothilone such as sagopilone, Ixabepilone or epothilone B, vinblastine, vinflunine, docetaxel and paclitaxel. Cytotoxic topoisomerase inhibiting agents include, e.g., aclarubicin, amonafide, belotecan, camptothecin, 10-hydroxycamptothecin, 9-aminocamptothecin, diflomotecan, irinotecan (Camptosar), edotecahn, epimbicin (Ellence), etoposide, exatecan, gimatecan, lurtotecan, mitoxantrone, pirambicin, pixantrone, rubitecan, sobuzoxane, tafluposide, and topotecan, and combinations thereof.

In a specific embodiment, an APMV described herein (e.g., a naturally occurring or recombinant APMV described herein) or a composition thereof is used in combination with interferon(s) or a composition thereof. Interferons include, e.g., interferon alpha, interferon alpha-2a, interferon alpha-2b, interferon beta, interferon gamma-1a, and interferon gamma-1b. In some embodiments, an APMV described herein (e.g., a naturally occurring or recombinant APMV described herein) or a composition thereof is used in combination with L19-IL2 or other L19 derivatives, filgrastim, lentinan, sizofilan, TheraCys, ubenimex, aldesleukin, alemtuzumab, BAM-002, dacarbazine, daclizumab, denileukin, gemtuzumab ozogamicin, ibritumomab, imiquimod, lenograstim, lentinan, melanoma vaccine (Corixa), molgramostim, sargramostim, tasonermin, tecleukin, thymalasin, tositumomab, Vimlizin, epratuzumab, mitumomab, oregovomab, pemtumomab, or Provenge.

In some embodiments, an APMV described herein (e.g., a naturally occurring or recombinant APMV described herein) or a composition thereof is used in combination with a biological response modifier(s), which is an agent that modifies defense mechanisms of living organisms or biological responses, such as survival, growth, or differentiation of tissue cells to direct them to have anti-tumor activity. In a specific embodiment, an APMV described herein (e.g., a naturally occurring or recombinant described herein) or a composition thereof is used in combination with a biological response modifier, such as krestin, lentinan, sizofiran, picibanil, ProMune or ubenimex.

In a specific embodiment, an APMV described herein (e.g., a naturally occurring or recombinant APMV described herein) or a composition thereof is used in combination with a pro-apoptotic agent(s), such as YM155, AMG 655, APO2L/TRAIL, or CHR-2797. In another specific embodiment, an APMV described herein (e.g., a naturally occurring or recombinant APMV described herein) or a composition thereof is used in combination with an anti-angiogenic compounds, such as, e.g., acitretin, Aflibercept, angiostatin, aplidine, asentar, Axitinib, Recentin, Bevacizumab, brivanib alaninat, cilengtide, combretastatin, DAST, endostatin, fenretinide, halofuginone, pazopanib, Ranibizumab, rebimastat, removab, Revlimid, Sorafenib, Vatalanib, squalamine, Sunitinib, Telatinib, thalidomide, ukrain, or Vitaxin.

In a specific embodiment, an APMV described herein (e.g., a naturally occurring or recombinant APMV described herein) or a composition thereof is used in combination with a platinum-coordinated compound, such as, e.g., cisplatin, carboplatin, nedaplatin, satraplatin or oxaliplatin. In another specific embodiment, an APMV described herein (e.g., a naturally occurring or recombinant APMV described herein) or a composition thereof is used in combination with a camptothecin derivative(s), such as, e.g., camptothecin, 10-hydroxycamptothecin, 9-aminocamptothecin, irinotecan, edotecarin, or topotecan.

In a specific embodiment, an APMV described herein (e.g., a naturally occurring or recombinant APMV described herein) or a composition thereof is used in combination with Trastuzumab, Cetuximab Bevacizumab, Rituximab, ticilimumab, Ipilimumab, lumiliximab, catumaxomab, atacicept; oregovomab, or alemtuzumab. In another specific embodiment, an APMV described herein (e.g., a naturally occurring or recombinant APMV described herein) or a composition thereof is used in combination with a VEGF inhibitor(s), such as, e.g., Sorafenib, DAST, Bevacizumab, Sunitinib, Recentin, Axitinib, Aflibercept, Telatinib, brivanib alaninate, Vatalanib, pazopanib or Ranibizumab.

In a specific embodiment, an APMV described herein (e.g., a naturally occurring or recombinant APMV described herein) or a composition thereof is used in combination with an EGFR (HER1) inhibitor(s), such as, e.g., Cetuximab, Panitumumab, Vectibix, Gefitinib, Erlotinib, or Zactima. In a specific embodiment, an APMV described herein (e.g., a naturally occurring or recombinant APMV described herein) or a composition thereof is used in combination with a HER2 inhibitor(s), such as, e.g., Lapatinib, Tratuzumab, or Pertuzumab.

In a specific embodiment, an APMV described herein (e.g., a naturally occurring or recombinant APMV described herein) or a composition thereof is used in combination with an mTOR inhibitor(s), such as, e.g., Temsirolimus, sirolimus/Rapamycin, or everolimus. In another specific embodiment, an APMV described herein (e.g., a naturally occurring or recombinant APMV described herein) or a composition thereof is used in combination with a cMet inhibitor(s). In another specific embodiment, an APMV described herein (e.g., a naturally occurring or recombinant APMV described herein) or a composition thereof is used in combination with a PI3K- and AKT inhibitor(s). In a specific embodiment, an APMV described herein (e.g., a naturally occurring or recombinant APMV described herein) or a composition thereof is used in combination with a CDK inhibitor(s), such as roscovitine or flavopiridol.

In a specific embodiment, an APMV described herein (e.g., a naturally occurring or recombinant APMV described herein) or a composition thereof is used in combination with a spindle assembly checkpoint inhibitor(s), targeted anti-mitotic drug or both. Examples of targeted anti-mitotic drugs are the PLK inhibitors and the Aurora inhibitors such as Hesperadin, checkpoint kinase inhibitors, and the KSP inhibitors.

In a specific embodiment, an APMV described herein (e.g., a naturally occurring or recombinant APMV described herein) or a composition thereof is used in combination with an HDAC inhibitor(s), such as, e.g., panobinostat, vorinostat, MS275, belinostat or LBH589. In another specific embodiment, an APMV described herein (e.g., a naturally occurring or recombinant APMV described herein) or a composition thereof is used in combination with an HSP90 inhibitor(s), HSP70 inhibitor(s) or both.

In a specific embodiment, an APMV described herein (e.g., a naturally occurring or recombinant APMV described herein) or a composition thereof is used in combination with a proteasome inhibitor(s), such as, e.g. bortezomib or carfilzomib. In another specific embodiment, an APMV described herein (e.g., a naturally occurring or recombinant APMV described herein) or a composition thereof is used in combination with a serine/threonine kinase inhibitor(s), such as, e.g., an MEK inhibitor(s) or Raf inhibitor(s) such as Sorafenib. In a specific embodiment, an APMV described herein (e.g., a naturally occurring or recombinant APMV described herein) or a composition thereof is used in combination with a farnesyl transferase inhibitor(s), e.g. tipifarnib.

In a specific embodiment, an APMV described herein (e.g., a naturally occurring or recombinant APMV described herein) or a composition thereof is used in combination with a tyrosine kinase inhibitor(s), such as, e.g., Dasatinib, Nilotibib, DAST, Bosutinib, Sorafenib, Bevacizumab, Sunitinib, AZD2171 , Axitinib, Aflibercept, Telatinib, imatinib mesylate, brivanib alaninate, pazopanib, Ranibizumab, Vatalanib, Cetuximab, Panitumumab, Vectibix, Gefitinib, Erlotinib, Lapatinib, Tratuzumab, Pertuzumab or c-Kit inhibitor(s). In another specific embodiment, an APMV described herein (e.g., a naturally occurring or recombinant APMV described herein) or a composition thereof is used in combination with a Vitamin D receptor agonist(s) or Bcl-2 protein inhibitor(s), such as, e.g, obatoclax, oblimersen sodium and gossypol.

In a specific embodiment, an APMV described herein (e.g., a naturally occurring or recombinant APMV described herein) or a composition thereof is used in combination with a cluster of differentiation 20 receptor antagonist(s), such as, e.g., rituximab. In another specific embodiment, an APMV described herein (e.g., a naturally occurring or recombinant APMV described herein) or a composition thereof is used in combination with a ribonucleotide reductase inhibitor, such as, e.g., Gemcitabine. In another specific embodiment, an APMV described herein (e.g., a naturally occurring or recombinant APMV described herein) or a composition thereof is used in combination with a Topoisomerase I and II Inhibitors, such as, e.g., Camptosar (Irinotecan) or doxorubicin.

In a specific embodiment, an APMV described herein (e.g., a naturally occurring or recombinant APMV described herein) or a composition thereof is used in combination with a Tumor Necrosis Apoptosis Inducing Ligand Receptor 1 Agonist(s), such as, e.g., mapatumumab. In another specific embodiment, an APMV described herein (e.g., a naturally occurring or recombinant APMV described herein) or a composition thereof is used in combination with a 5-Hydroxytryptamine Receptor Antagonist(s), such as, e.g., rEV598, Xaliprode, Palonosetron hydrochloride, granisetron, Zindol, palonosetron hydrochloride or AB-1001.

In a specific embodiment, an APMV described herein (e.g., a naturally occurring or recombinant APMV described herein) or a composition thereof is used in combination with an integrin inhibitor(s), such as, e.g., Alpha-5 Beta-1 integrin inhibitors such as E7820, JSM 6425, volociximab or Endostatin. In a specific embodiment, an APMV described herein (e.g., a naturally occurring or recombinant APMV described herein) or a composition thereof is used in combination with an androgen receptor antagonist(s), such as, e.g., nandrolone decanoate, fluoxymesterone, fluoxymesterone, Android, Prost-aid, Andromustine, Bicalutamide, Flutamide, Apo-Cyproterone, Apo-Flutamide, chlormadinone acetate, bicalutamide, Androcur, Tabi, cyproterone acetate, Cyproterone Tablets, or nilutamide. In another specific embodiment, an APMV described herein (e.g., a naturally occurring or recombinant APMV described herein) or a composition thereof is used in combination with an aromatase inhibitor(s), such as, e.g., anastrozole, letrozole, testolactone, exemestane, Aminoglutethimide or formestane. In another specific embodiment, an APMV described herein (e.g., a naturally occurring or recombinant APMV described herein) or a composition thereof is used in combination with a Matrix metalloproteinase inhibitor(s). In another specific embodiment, an APMV described herein (e.g., a naturally occurring or recombinant APMV described herein) or a composition thereof is used in combination with alitretinoin, ampligen, atrasentan bexarotene, bortezomib, bosentan, calcitriol, exisulind, finasteride, fotemustine, ibandronic acid, miltefosine, mitoxantrone, 1-asparaginase, procarbazine, dacarbazine, hydroxycarbamide, hydroxycarbamide, pegaspargase, pentostatin, tazarotne, velcade, gallium nitrate, Canfosfamide darinaparsin or tretinoin.

Currently available cancer therapies and their dosages, routes of administration and recommended usage are known in the art and have been described in such literature as the Physicians' Desk Reference (71st ed., 2017).

### 5.5.3 Patient Population

In some embodiments, an APMV described herein (e.g., a naturally occurring or recombinant APMV described herein) or a composition thereof, or a combination therapy described herein is administered to a subject suffering from cancer. In other embodiments, an APMV described herein (e.g., a naturally occurring or recombinant APMV described herein) or a composition thereof, or a combination therapy described herein is administered to a subject predisposed or susceptible to cancer. In some embodiments, an APMV described herein (e.g., a naturally occurring or recombinant APMV described herein) or a composition thereof, or a combination therapy described herein is administered to a subject diagnosed with cancer. Specific examples of the types of cancer are described herein (see, e.g., Section 5.5.4 and Section 6). In an embodiment, the subject has metastatic cancer. In another embodiment, the subject has stage 1, stage 2, stage 3, or stage 4 cancer. In another embodiment, the subject is in remission. In yet another embodiment, the subject has a recurrence of cancer.

In certain embodiments, an APMV described herein (e.g., a naturally occurring or recombinant APMV described herein) or a composition thereof, or a combination therapy described herein is administered to a human that is 0 to 6 months old, 6 to 12 months old, 6 to 18 months old, 18 to 36 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In some embodiments, a an APMV described herein (e.g., a naturally occurring or recombinant APMV described herein) or a composition thereof, or a combination therapy described herein is administered to a human infant. In other embodiments, an APMV described herein (e.g., a naturally occurring or recombinant APMV described herein) or a composition thereof, or a combination therapy described herein is administered to a human toddler. In other embodiments, an APMV described herein (e.g a naturally occurring or recombinant APMV described herein) or a composition thereof, or a combination therapy described herein is administered to a human child. In other embodiments, an APMV described herein (e.g., a naturally occurring or recombinant APMV described herein) or a composition thereof, or a combination therapy described herein is administered to a human adult. In yet other embodiments, an APMV described herein (e.g., a naturally occurring or recombinant APMV described herein) or a composition thereof, or a combination therapy described herein is administered to an elderly human.

In certain embodiments, an APMV described herein (e.g., a naturally occurring or recombinant APMV described herein) or a composition thereof, or a combination therapy described herein is administered to a subject in an immunocompromised state or immunosuppressed state or at risk for becoming immunocompromised or immunosuppressed. In certain embodiments, an APMV described herein (e.g., a naturally occurring or recombinant APMV described herein) or a composition thereof, or a combination therapy described herein is administered to a subject receiving or recovering from immunosuppressive therapy. In certain embodiments, an APMV described herein (e.g., a naturally occurring or recombinant APMV described herein) or a composition thereof, or a combination therapy described herein is administered to a subject that has or is at risk of getting cancer. In certain embodiments, the subject is, will or has undergone surgery, chemotherapy and/or radiation therapy. In certain embodiments, the patient has undergone surgery to remove the tumor or neoplasm. In specific embodiments, the patient is administered an APMV described herein (e.g., a naturally occurring or recombinant APMV described herein) or a composition thereof, or a combination therapy described herein following surgery to remove a tumor or neoplasm. In other embodiments, the patient is administered an APMV described herein (e.g., a naturally occurring or recombinant APMV described herein) or a composition thereof, or a combination therapy described herein prior to undergoing surgery to remove a tumor or neoplasm. In certain embodiments, an APMV described herein (e.g., a naturally occurring or recombinant APMV described herein) or a composition thereof, or a combination therapy described herein is administered to a subject that has, will have or had a tissue transplant, organ transplant or transfusion.

In some embodiments, an APMV described herein (e.g., a naturally occurring or recombinant APMV described herein) or a composition thereof, or a combination therapy described herein is administered to a patient who has proven refractory to therapies other than the APMV or composition thereof, or a combination therapy but are no longer on these therapies. In a specific embodiment, an APMV described herein (e.g., a naturally occurring or recombinant APMV described herein) or a composition thereof, or a combination therapy described herein is administered to a patient who has proven refractory to chemotherapy. The determination of whether cancer is refractory can be made by any method known in the art. In a certain embodiment, refractory patient is a patient refractory to a standard therapy. In some embodiments, a patient with cancer is initially responsive to therapy, but subsequently becomes refractory.

### 5.5.4 Types of Cancers

Specific examples of cancers that can be treated in accordance with the methods described herein include, but are not limited to: melanomas, leukemias, lymphomas, multiple myelomas, sarcomas, and carcinomas. In one embodiment, cancer treated in accordance with the methods described herein is a leukemia, such as acute leukemia, acute lymphocytic leukemia, acute myelocytic leukemias, such as, myeloblastic, promyelocytic, myelomonocytic, monocytic, erythroid leukemias, and myelodysplastic syndrome. In another embodiment, cancer treated in accordance with the methods described herein is a chronic leukemia, such as chronic myelocytic (granulocytic) leukemia, chronic lymphocytic leukemia, and hairy cell leukemia. In another embodiment, cancer treated in accordance with the methods described herein is a lymphoma, such as Hodgkin disease and non-Hodgkin disease. In another embodiment, cancer treated in accordance with the methods described herein is a multiple myeloma such as smoldering multiple myeloma, nonsecretory myeloma, osteosclerotic myeloma, solitary plasmacytoma and extramedullary plasmacytoma. In another embodiment, cancer treated in accordance with the methods described herein is Waldenstrom's macroglobulinemia monoclonal gammopathy of undetermined significance, benign monoclonal gammopathy, Wilm's tumor, or heavy chain disease.

In one embodiment, cancer treated in accordance with the methods described herein is bone cancer, brain cancer, breast cancer, adrenal cancer, thyroid cancer, pancreatic cancer, pituitary cancer, eye cancer, vaginal, vulvar cancer, cervical cancer, uterine cancer, ovarian cancer, esophageal cancer, stomach cancer, colon cancer, rectal cancer, liver cancer, gallbladder cancer, lung cancer, testicular cancer, prostate cancer, penal cancer, oral cancer, basal cancer, salivary gland cancer, pharynx cancer, skin cancer, kidney cancer, or bladder cancer. In another embodiment, cancer treated in accordance with the methods described herein is brain, breast, lung, colorectal, liver, kidney or skin cancer.

In another embodiment, cancer treated in accordance with the methods described herein is a bone and connective tissue sarcoma, such as bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, or synovial sarcoma. In another embodiment, cancer treated in accordance with the methods described herein is a brain tumor, such as glioma, astrocytoma, brain stem glioma, ependymoma, oligodendroglioma, nonglial tumor, glioblastoma multiforme, acoustic neurinoma, craniopharyngioma, medulloblastoma, meningioma, pineocytoma, pineoblastoma, or primary brain lymphoma. In another embodiment, cancer treated in the accordance with the methods described herein is breast cancer, such as triple negative breast cancer, ER+/HER2-breast cancer, ductal carcinoma, adenocarcinoma, lobular (cancer cell) carcinoma, intraductal carcinoma, medullary breast cancer, mucinous breast cancer, tubular breast cancer, papillary breast cancer, Paget's disease, or inflammatory breast cancer. In another embodiment, cancer treated in the accordance with the methods described herein is adrenal cancer, such as pheochromocytom or adrenocortical carcinoma. In another embodiment, cancer treated in the accordance with the methods described herein is thyroid cancer, such as papillary or follicular thyroid cancer, medullary thyroid cancer or anaplastic thyroid cancer. In another embodiment, cancer treated in the accordance with the methods described herein is pancreatic cancer, such as insulinoma, gastrinoma, glucagonoma, vipoma, somatostatin-secreting tumor, or carcinoid or islet cell tumor. In another embodiment, cancer treated in the accordance with the methods described herein is pituitary cancer, such as Cushing's disease, prolactin-secreting tumor, acromegaly, or diabetes insipidus. In another embodiment, cancer treated in the accordance with the methods described herein is eye cancer, such as ocular melanoma such as iris melanoma, choroidal melanoma, cilliary body melanoma, or retinoblastoma. In another embodiment, cancer treated in the accordance with the methods described herein is vaginal cancer, such as squamous cell carcinoma, adenocarcinoma, or melanoma. In another embodiment, cancer treated in the accordance with the methods described herein is vulvar cancer, such as squamous cell carcinoma, melanoma, adenocarcinoma, basal cell carcinoma, sarcoma, or Paget's disease. In another embodiment, cancer treated in the accordance with the methods described herein is cervical cancer, such as squamous cell carcinoma or adenocarcinoma. In another embodiment, cancer treated in the accordance with the methods described herein is uterine cancer, such as endometrial carcinoma or uterine sarcoma.

In another embodiment, cancer treated in accordance with the methods described herein is ovarian cancer, such as ovarian epithelial carcinoma, borderline tumor, germ cell tumor, or stromal tumor. In another embodiment, cancer treated in accordance with the methods described herein is esophageal cancer, such as squamous cancer, adenocarcinoma, adenoid cystic carcinoma, mucoepidermoid carcinoma, adenosquamous carcinoma, sarcoma, melanoma, placancercytoma, verrucous carcinoma, or oat cell (cancer cell) carcinoma. In another embodiment, cancer treated in accordance with the methods described herein is stomach cancer, such as adenocarcinoma, fungating (polypoid), ulcerating, superficial spreading, diffusely spreading, malignant lymphoma, liposarcoma, fibrosarcoma, or carcinosarcoma. In another embodiment, cancer treated in accordance with the methods described herein is liver cancer, such as hepatocellular carcinoma or hepatoblastoma. In another embodiment, cancer treated in accordance with the methods described herein is gallbladder cancer, such as adenocarcinoma. In another embodiment, cancer treated in accordance with the methods described herein is cholangiocarcinoma, such as papillary, nodular, or diffuse. In another embodiment, cancer treated in accordance with the methods described herein is lung cancer, such as non-small cell lung cancer, squamous cell carcinoma (epidermoid carcinoma), adenocarcinoma, large-cell carcinoma or cancer-cell lung cancer. In another embodiment, cancer treated in accordance with the methods described herein is testicular cancer, such germinal tumor, seminoma, anaplastic, classic (typical), spermatocytic, nonseminoma, embryonal carcinoma, teratoma carcinoma, or choriocarcinoma (yolk-sac tumor). In another embodiment, cancer treated in accordance with the methods described herein is prostate cancer, such as prostatic intraepithelial neoplasia, adenocarcinoma, leiomyosarcoma, or rhabdomyosarcoma. In another embodiment, cancer treated in accordance with the methods described herein is penal cancers. In another embodiment, cancer treated in accordance with the methods described herein is oral cancer, such as squamous cell carcinoma. In another embodiment, cancer treated in accordance with the methods described herein is salivary gland cancer, such as adenocarcinoma, mucoepidermoid carcinoma, or adenoidcystic carcinoma. In another embodiment, cancer treated in accordance with the methods described herein is pharynx cancer, such as squamous cell cancer or verrucous. In another embodiment, cancer treated in accordance with the methods described herein is skin cancer, such as basal cell carcinoma, squamous cell carcinoma and melanoma, superficial spreading melanoma, nodular melanoma, lentigo malignant melanoma, or acral lentiginous melanoma. In another embodiment, cancer treated in accordance with the methods described herein is kidney cancer, such as renal cell carcinoma, adenocarcinoma, hypernephroma, fibrosarcoma, or transitional cell cancer (renal pelvis and/or uterine). In another embodiment, cancer treated in accordance with the methods described herein is bladder cancer, such as transitional cell carcinoma, squamous cell cancer, adenocarcinoma, or carcinosarcoma.

In a specific embodiment, the cancer treated in accordance with the methods described herein is a melanoma. In another specific embodiment, the cancer treated in accordance with the methods described herein is a lung carcinoma. In another specific embodiment, the cancer treated in accordance with the methods described herein is a colorectal carcinoma. In a specific embodiment, the cancer treated in accordance with the methods described herein is melanoma, non-small cell lung cancer, head and neck squamous cell cancer, classical Hodgkin lymphoma, primary mediastinal large B-cell lymphoma, urothelial carcinoma, microsatellite instability-high cancer, gastric cancer, or cervical cancer.

In a specific embodiment, an APMV described herein or compositions thereof, or a combination therapy described herein are useful in the treatment of a variety of cancers and abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma.

In some embodiments, cancers associated with aberrations in apoptosis are treated in accordance with the methods described herein. Such cancers may include, but are not limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders of the skin, lung, liver, bone, brain, stomach, colon, breast, prostate, bladder, kidney, pancreas, ovary, uterus or any combination of the foregoing are treated in accordance with the methods described herein. In other specific embodiments, a sarcoma or melanoma is treated in accordance with the methods described herein.

In a specific embodiment, the cancer being treated in accordance with the methods described herein is leukemia, lymphoma or myeloma (e.g., multiple myeloma). Specific examples of leukemias and other blood-borne cancers that can be treated in accordance with the methods described herein include, but are not limited to, acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, and hairy cell leukemia.

Specific examples of lymphomas that can be treated in accordance with the methods described herein include, but are not limited to, Hodgkin disease, non-Hodgkin lymphoma such as diffuse large B-cell lymphoma, multiple myeloma, Waldenstrom's macroglobulinemia, heavy chain disease, and polycythemia vera.

In another embodiment, the cancer being treated in accordance with the methods described herein is a solid tumor. Examples of solid tumors that can be treated in accordance with the methods described herein include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, cancer cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma. In another embodiment, the cancer being treated in accordance with the methods described herein is a metastatic. In another embodiment, the cancer being treated in accordance with the methods described herein is malignant.

In a specific embodiment, the cancer being treated in accordance with the methods described herein is a cancer that has a poor prognosis and/or has a poor response to conventional therapies, such as chemotherapy and radiation. In another specific embodiment, the cancer being treated in accordance with the methods described herein is malignant melanoma, malignant glioma, renal cell carcinoma, pancreatic adenocarcinoma, malignant pleural mesothelioma, lung adenocarcinoma, lung small cell carcinoma, lung squamous cell carcinoma, anaplastic thyroid cancer, or head and neck squamous cell carcinoma. In another specific embodiment, the cancer being treated in accordance with the methods described herein is a type of cancer described in Section 6, infra.

In a specific embodiment, the cancer being treated in accordance with the methods described herein is a cancer that is metastatic. In a specific embodiment, the cancer comprises a dermal, subcutaneous, or nodal metastasis. In a specific embodiment, the cancer comprises peritoneal or pleural metastasis. In a specific embodiment, the cancer comprises visceral organ metastasis, such as liver, kidney, spleen, or lung metastasis.

In a specific embodiment, the cancer being treated in accordance with the methods described herein is a cancer that is unresectable. Any method known to the skilled artisan may be utilized to determine if a cancer is unresectable.

### 5.6 Biological Assays

In a specific embodiment, one, two or more of the assays described in Section 6 may be used to characterize an APMV described herein.

### 5.6.1 In Vitro Assays

Viral assays include those that indirectly measure viral replication (as determined, e.g., by plaque formation) or the production of viral proteins (as determined, e.g., by western blot analysis) or viral RNAs (as determined, e.g., by RT-PCR or northern blot analysis) in cultured cells in vitro using methods which are well known in the art.

Growth of an APMV described herein can be assessed by any method known in the art or described herein (e.g., in cell culture (e.g., cultures of chicken embryonic kidney cells or cultures of chicken embryonic fibroblasts (CEF)) (see, e.g., Section 6). Viral titer may be determined by inoculating serial dilutions of a recombinant APMV described herein into cell cultures (e.g., CEF, MDCK, EFK-2 cells, Vero cells, primary human umbilical vein endothelial cells (HUVEC), H292 human epithelial cell line or HeLa cells), chick embryos, or live animals (e.g., avians). After incubation of the virus for a specified time, the virus is isolated using standard methods. Physical quantitation of the virus titer can be performed using PCR applied to viral supernatants (Quinn & Trevor, 1997; Morgan et al., 1990), hemagglutination assays, tissue culture infectious doses (TCID50) or egg infectious doses (EID50). An exemplary method of assessing viral titer is described in Section 6, below.

Incorporation of nucleotide sequences encoding a heterologous peptide or protein (e.g., a transgene into the genome of an APMV described herein can be assessed by any method known in the art or described herein (e.g., in cell culture, an animal model or viral culture in embryonated eggs)). For example, viral particles from cell culture of the allantoic fluid of embryonated eggs can be purified by centrifugation through a sucrose cushion and subsequently analyzed for protein expression by Western blotting using methods well known in the art.

Immunofluorescence-based approaches may also be used to detect virus and assess viral growth. Such approaches are well known to those of skill in the art, e.g., fluorescence microscopy and flow cytometry (see, eg., Section 6, infra). Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, Oreg.; Sigma-Aldrich (2003) Catalogue, St. Louis, Mo.). See, e.g., the assays described in Section 6, infra.

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.). See also Section 6, infra, for histology and immunohistochemistry assays that may be used.

### 5.6.2 Interferon Assays

IFN induction and release by an APMV described herein may be determined using techniques known to one of skill in the art. For example, the amount of IFN induced in cells following infection with a recombinant APMV described herein may be determined using an immunoassay (e.g., an ELISA or Western blot assay) to measure IFN expression or to measure the expression of a protein whose expression is induced by IFN. Alternatively, the amount of IFN induced may be measured at the RNA level by assays, such as Northern blots and quantitative RT-PCR, known to one of skill in the art. In specific embodiments, the amount of IFN released may be measured using an ELISPOT assay. Further, the induction and release of cytokines and/or interferon-stimulated genes may be determined by, e.g., an immunoassay or ELISPOT assay at the protein level and/or quantitative RT-PCR or northern blots at the RNA level.

### 5.6.3 Activation Marker Assays and Immune Cell Infiltration Assay

The expression of a T cell marker, B cell marker, activation marker, co-stimulatory molecule, ligand, or inhibitory molecule by immune cells induced by an APMV may be assessed. Techniques for assessing the expression of T cell marker, B cell marker, activation marker, co-stimulatory molecule, ligand, or inhibitory molecule by immune cells are known to one of skill in the art. For example, the expression of T cell marker, B cell marker, an activation marker, co-stimulatory molecule, ligand, or inhibitory molecule by an immune cell can be assessed by flow cytometry.

### 5.6.4 Toxicity Studies

In some embodiments, an APMV described herein or composition thereof, or a combination therapy described herein are tested for cytotoxicity in mammalian, preferably human, cell lines. In certain embodiments, cytotoxicity is assessed in one or more of the following non-limiting examples of cell lines: U937, a human monocyte cell line; primary peripheral blood mononuclear cells (PBMC); Huh7, a human hepatoblastoma cell line; HL60 cells, HT1080, HEK 293T and 293H, MLPC cells, human embryonic kidney cell lines; human melanoma cell lines, such as SkMel2, SkMel-119 and SkMel-197; THP-1, monocytic cells; a HeLa cell line; and neuroblastoma cells lines, such as MC-IXC, SK-N-MC, SK-N-MC, SK-N-DZ, SH-SY5Y, and BE(2)-C. In some embodiments, the ToxLite assay is used to assess cytotoxicity.

Many assays well-known in the art can be used to assess viability of cells or cell lines following infection with an APMV described herein or composition thereof, and, thus, determine the cytotoxicity of the APMV or composition thereof. For example, cell proliferation can be assayed by measuring Bromodeoxyuridine (BrdU) incorporation, (³H) thymidine incorporation, by direct cell count, or by detecting changes in transcription, translation or activity of known genes such as proto-oncogenes (e.g., fos, myc) or cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as ELISA, Western blotting or immunoprecipitation using antibodies, including commercially available antibodies. mRNA can be quantitated using methods that are well known and routine in the art, for example, using northern analysis, RNase protection, or polymerase chain reaction in connection with reverse transcription. Cell viability can be assessed by using trypan-blue staining or other cell death or viability markers known in the art. In a specific embodiment, the level of cellular ATP is measured to determined cell viability. In preferred embodiments, an APMV described herein or composition thereof does not kill healthy (i.e., non-cancerous) cells.

In specific embodiments, cell viability may be measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes may include enlargement, granularity, cells with ragged edges, a filmy appearance, rounding, detachment from the surface of the well, or other changes.

The APMVs described herein or compositions thereof, or combination therapies can be tested for in vivo toxicity in animal models. For example, animal models, known in the art to test the effects of compounds on cancer can also be used to determine the in vivo toxicity of an APMV described herein or a composition thereof, or combination therapies. For example, animals are administered a range of pfu of an APMV described herein, and subsequently, the animals are monitored over time for various parameters, such as one, two or more of the following: lethality, weight loss or failure to gain weight, and levels of serum markers that may be indicative of tissue damage (e.g., creatine phosphokinase level as an indicator of general tissue damage, level of glutamic oxalic acid transaminase or pyruvic acid transaminase as indicators for possible liver damage). These in vivo assays may also be adapted to test the toxicity of various administration mode and regimen in addition to dosages.

The toxicity, efficacy or both of an APMV described herein or a composition thereof, or a combination therapy described herein can be determined by standard pharmaceutical procedures in cell cultures or experimental animals. In a specific embodiment, the cytotoxicity of an APMV is determined by methods set forth in Section 6, infra.

The data obtained from the cell culture assays and animal studies can be used in formulating a range of dosage of the therapies for use in subjects.

### 5.6.5 Biological Activity Assays

An APMV described herein or a composition thereof, or a combination therapy described herein can be tested for biological activity using animal models for treating cancer. (see, e.g., Section 6). Such animal model systems include, but are not limited to, rats, mice, hamsters, cotton rats, chicken, cows, monkeys (e.g., African green monkey), pigs, dogs, rabbits, etc. In a specific embodiment, an animal model such as described in Section 6, infra, is used to test the utility of an APMV or composition thereof to treat cancer.

### 5.6.6 Expression of Transgene

The expression of a protein in cells infected with a recombinant APMV described herein, wherein the recombinant APMV comprises a packaged genome comprising a transgene encoding a heterologous protein, may be conducted using any assay known in the art, such as, e.g., western blot, immunofluorescence, flow cytometry, and ELISA, or any assay described herein (see, e.g., Section 6).

In a specific aspect, an ELISA is utilized to detect expression of a heterologous protein encoded by a transgene in cells infected with a recombinant APMV comprising a packaged genome comprising the transgene.

The expression of a transgene may also be measured at the RNA level by assays, such as Northern blots and quantitative RT-PCR, known to one of skill in the art.

In addition to expression of a transgene, the function of the protein encoded by the transgene may be assessed by techniques known to one of skill in the art. For example, one or more functions of a protein described herein or known to one of skill in the art may be assessed using techniques known to one of skill in the art.

### 5.7 Kits

In one aspect, provided herein is a pharmaceutical pack or kit comprising one or more containers filled with one or more of the ingredients of a composition (e.g., a pharmaceutical compositions) described herein. In a specific embodiment, provided herein is a pharmaceutical pack or kit comprising a container, wherein the container comprises an APMV (e.g., AMP-2, APMV-3, APMV-4, APMV-6, APMV-7, APMV-8 or APMV-9) described herein, or a pharmaceutical composition comprising an APMV (e.g., AMP-2, APMV-3, APMV-4, APMV-6, APMV-7, APMV-8 or APMV-9) described herein. In a particular embodiment, provided herein is a pharmaceutical pack or kit comprising a container, wherein the container comprises an APMV-4 described herein, or a pharmaceutical composition comprising an APMV-4 described herein. In certain embodiments, the pharmaceutical pack or kit comprises a second container, wherein the second container comprises an additional prophylactic or therapeutic agent, such as, e.g., described in Section 5.5.2. Optionally associated with such container(s) can be a notice in the form prescribed by a governmental agency regulating the manufacture, use or sale of pharmaceuticals or biological products, which notice reflects approval by the agency of manufacture, use or sale for human administration. In a specific embodiment, the pharmaceutical pack or kit includes instructions for use of the APMV or composition thereof for the treatment of cancer.

TABLE 2
SEQUENCES
APMV SEQUENCES

| | | SEQ ID |
|---|---|---|
| Description | Sequence | NO. |
| Avian | ACCAAACAAGGAATAGGTAAGCAAC | SEQ ID |
| paramyxovir | GTAAATCTTAGATAAAACCATAGAA | NO: 1 |
| us 2 strain | TCCGTGGGGGCGACATCGCCTGAAG | |
| APMV- | CCGATCTCGAGATCGATAACTCCGG | |
| 2/Chicken/ | TTAATTGGTCTCAGCGTGAGGAGCT | |
| California/ | TATCTGTCTGTGGCAATGTCTTCTG | |
| Yucaipa/56, | TGTTTTCAGAATACCAGGCTCTTCA | |
| complete | GGACCAACTGGTCAAGCCTGCCACT | |
| genome | CGAAGGGCTGATGTGGCATCGACTG | |
| Genbank: | GATTGTTGAGAGCGGAGATACCAGT | |
| EU338414.1 | TTGTGTAACCTTGTCTCAGGACCCA | |
| | ACTGATAGATGGAACCTCGCATGTC | |
| | TCAATCTGCGATGGCTGATAAGTGA | |
| | GTCCTCTACTACTCCCATGAGACAA | |
| | GGGGCGATCCTGTCACTGCTGAGCT | |
| | TGCACTCTGACAACATGCGAGCTCA | |
| | CGCAACCCTTGCAGCGAGATCCGCT | |
| | GATGCTGCCATCACTGTGCTTGAGG | |
| | TTGACGCCATAGACATGGCGGATGG | |
| | CACAATCACTTTTAATGCCAGAAGT | |
| | GGAGTATCCGAGAGGCGCAGCACAC | |
| | AGCTCATGGCAATCGCAAAAGATCT | |
| | GCCCCGCTCTTGTTCCAATGACTCA | |
| | CCATTCAAAGATGACACTATCGAGG | |
| | ATCGCGACCCCCTTGACCTGTCCGA | |
| | GACTATCGATAGACTGCAGGGGATT | |
| | GCTGCCCAAATCTGGATAGCGGCCA | |
| | TCAAGAGCATGACTGCCCCGGATAC | |
| | TGCTGCGGAGTCAGAAGGCAAGAGG | |
| | CTTGCAAAGTACCAACAACAAGGCC | |
| | GCTTGGTGCGACAGGTGTTAGTGCA | |
| | TGATGCGGTGCGTGCGGAATTCCTA | |
| | CGTGTCATCAGAGGCAGCCTGGTCT | |
| | TACGGCAATTCATGGTATCAGAATG | |
| | TAAGAGGGCAGCATCCATGGGTAGC | |
| | GAGACATCTAGGTACTATGCCATGG | |
| | TGGGTGACATCAGCCTCTACATCAA | |
| | GAATGCAGGACTTACCGCCTTCTTC | |
| | TTGACACTCAGATTTGGTATTGGGA | |
| | CACACTACCCCACTCTTGCCATGAG | |
| | TGTGTTCTCTGGAGAACTGAAGAAG | |
| | ATGTCGTCCTTGATCAGGCTGTATA | |
| | AGTCAAAAGGGGAAAATGCTGCATA | |
| | CATGGCATTCCTGGAGGATGCGGAC | |
| | ATGGGAAACTTTGCGCCTGCTAACT | |
| | TTAGTACTCTCTACTCCTATGCAAT | |
| | GGGGGTAGGTACAGTGCTGGAAGCA | |
| | TCAGTTGCGAAATACCAGTTCGCTC | |
| | GAGAGTTCACCAGTGAGACATACTT | |
| | CAGGCTTGGGGTTGAGACCGCACAG | |
| | AACCAACAGTGCGCTCTAGATGAAA | |
| | AGACCGCCAAGGAGATGGGGCTTAC | |
| | TGATGAAGCCAGAAAGCAGGTGCAA | |
| | GCATTGGCTAGCAACATCGAGCAGG | |
| | GGCAACATTCAATGCCCATGCAACA | |
| | ACAGCCCACATTCATGAGTCAGCCC | |
| | TACCAGGATGACGATCGTGACCAGC | |
| | CAAGCACCAGCAGACCAGAGCCAAG | |
| | ACCATCGCAATTGACAAGCCAATCA | |
| | GCAGCACAGGACAATGATGCGGCCT | |
| | CATTAGATTGGTGACCGCAATCAGC | |
| | TCAGCCAAGCCATTGTTGGACGCAG | |
| | GACATTCAAATCATACATTGCCCTA | |
| | AGAGTATTAAAGTGATTTAAGAAAA | |
| | AAGGACCCTGGGGGCGAAGTTGTCC | |
| | CAATCCAGGCAGGCGCTGAAACCGA | |
| | ATCCCTCCAACCTCCGAGCCCCAGG | |
| | CGACCATGGAGTTCACCGATGATGC | |
| | CGAAATTGCTGAGCTGTTGGACCTC | |
| | GGGACCTCAGTGATCCAAGAGCTGC | |
| | AGCGAGCCGAAGTCAAGGGCCCGCA | |
| | AACAACCGGAAAGCCCAAAGTTCCC | |
| | CCGGGGAACACTAAGAGCCTGGCTA | |
| | CTCTCTGGGAGCATGAGACTAGCAC | |
| | CCAAGGGAGTGCATTGGGCACACCC | |
| | GAGAACAACACCCAGGCACCCGATG | |
| | ACAACAACGCAGGTGCAGATACGCC | |
| | AGCGACTACCGACGTCCATCGCACT | |
| | CTGGATACCATAGACACCGACACAC | |
| | CACCGGAAGGGAGCAAGCCCAGCTC | |
| | CACTAACTCCCAACCCGGTGATGAC | |
| | CTTGACAAGGCTCTTTCGAAGCTAG | |
| | AGGCGCGCGCCAAGCTCGGACCAGA | |
| | TAGGGCCAGACAGGTTAAAAAGGGG | |
| | AAGGAGATCGGGTCGAGCACAGGGA | |
| | CGAGGGAGGCAGCCAGTCACCACAT | |
| | GGAAGGGAGCCGACAGTCGGAGCCA | |
| | GGAGCGGGCAGCCGAGCACAGCCAC | |
| | AAGGCCATGGCGACCGGGACACAGG | |
| | AGGGAGTACTCATTCATCTCTCGAG | |
| | ATGGGAGACTGGAAGTCACAAGCTG | |
| | GTGCAACCCAGTCTGCTCTCCCATT | |
| | AGAAGCGAGCCCAGGAGAGAAAAGT | |
| | GCACATGTGGAACTTGCCCAGAATC | |
| | CTGCATTTTATGCAGGCAACCCAAC | |
| | TGATGCAATTATGGGGTTGACAAAG | |
| | AAAGTCAATGATCTAGAGACAAAAT | |
| | TGGCTGAGGTATTGCGTCTGTTAGG | |
| | AATACTCCCCGGAATAAAGAATGAG | |
| | ATTAGTCAGCTGAAAGCAACCGTGG | |
| | CTCTGATGTCAAATCAGATTGCCTC | |
| | CATTCAGATTCTTGATCCTGGGAAT | |
| | GCCGGAGTCAAATCCCTTAATGAGA | |
| | TGAAAGCCCTGTCAAAAGCAGCCAG | |
| | CATAGTTGTGGCAGGTCCAGGAGTC | |
| | CTTCCTCCTGAGGTCACAGAAGGAG | |
| | GACTGATCGCGAAAGATGAGCTAGC | |
| | AAGGCCCATCCCCATCCAACCGCAA | |
| | CGAGACTCCAAACCCAAAGACGACC | |
| | CGCACACATCACCAAATGATGTCCT | |
| | TGCTGTACGCGCTATGATCGACACC | |
| | CTTGTGGATGATGAGAAGAAGAGAA | |
| | AGAGATTAAACCAGGCCCTTGACAA | |
| | GGCAAAGACCAAGGATGACGTCTTA | |
| | AGGGTCAAGCGGCAGATATACAATG | |
| | CCTAGGAGTCCATTTGTCTAAAGAA | |
| | CCTCCAATCATATCACCAGTTTCGT | |
| | GCCACATGCTTCCCTGCCGAGAATC | |
| | TAGCCGACACAAAAACTAAATCATA | |
| | GTTTAACAAAAAAGAAGTTTGGGGG | |
| | CGAAGTCTCACATCATAGAGCACCC | |
| | TTGCATTCTAAAATGGCTCAAACAA | |
| | CCGTCAGGCTGTATATCGATGAAGC | |
| | TAGTCCCGACATTGAACTGTTGTCT | |
| | TACCCACTGATAATGAAAGACACAG | |
| | GACATGGGACCAAAGAGTTGCAGCA | |
| | GCAAATCAGAGTTGCAGAGATCGGT | |
| | GCATTGCAGGGAGGGAAGAATGAAT | |
| | CAGTTTTCATCAATGCATATGGCTT | |
| | TGTTCAGCAATGCAAAGTTAAACCG | |
| | GGGGCAACCCAATTCTTCCAGGTAG | |
| | ATGCAGCTACAAAGCCAGAAGTGGT | |
| | CACTGCAGGGATGATTATAATCGGT | |
| | GCAGTCAAGGGGGTGGCAGGCATCA | |
| | CTAAGCTGGCAGAAGAGGTGTTCGA | |
| | GCTGGACATCTCCATCAAGAAGTCC | |
| | GCATCATTCCATGAGAAGGTTGCGG | |
| | TGTCCTTTAATACTGTGCCACTATC | |
| | ACTCATGAATTCGACCGCATGCAGA | |
| | AATCTGGGTTATGTCACAAACGCTG | |
| | AGGAGGCGATCAAATGCCCGAGCAA | |
| | AATACAAGCGGGTGTGACGTACAAA | |
| | TTTAAGATAATGTTTGTCTCCTTGA | |
| | CACGACTGCATAACGGGAAATTGTA | |
| | CCGTGTCCCCAAGGCAGTGTATGCT | |
| | GTAGAGGCATCAGCTCTATATAAAG | |
| | TGCAACTGGAAGTCGGGTTCAAGCT | |
| | TGACGTGGCCAAGGATCACCCACAC | |
| | GTTAAGATGTTGAAGAAAGTGGAAC | |
| | GGAATGGTGAGACTCTGTATCTTGG | |
| | TTATGCATGGTTCCACCTGTGCAAC | |
| | TTCAAGAAGACAAATGCCAAGGGTG | |
| | AGTCCCGGACAATCTCCAACCTAGA | |
| | AGGGAAAGTCAGAGCTATGGGGATC | |
| | AAGGTTTCCTTGTACGACTTATGGG | |
| | GGCCTACTTTGGTGGTGCAAATCAC | |
| | AGGTAAGACCAGCAAGTATGCACAA | |
| | GGTTTCTTTTCAACCACAGGTACCT | |
| | GCTGCCTCCCAGTGTCGAAGGCTGC | |
| | CCCTGAGCTGGCCAAACTTATGTGG | |
| | TCCTGCAATGCAACAATCGTTGAAG | |
| | CTGCAGTGATTATCCAAGGGAGTGA | |
| | TAGGAGGGCAGTCGTGACCTCAGAG | |
| | GACTTGGAAGTATACGGGGCAGTTG | |
| | CAAAAGAGAAGCAGGCTGCAAAAGG | |
| | ATTTCACCCGTTCCGCAAGTGACAC | |
| | GTGGGGCCGCACACCTCATTACCCC | |
| | AGAAGCCCGGGCAACTGCAAATTCA | |
| | CGCTTATATAATCCAATTACCATGA | |
| | TCTAGAACTGCAATCGATACTAATC | |
| | GCTCATTGATCGTATTAAGAAAAAA | |
| | CTTAACTACATAACTTCAACATTGG | |
| | GGGCGACAGCTCCAGACTAAGTGGG | |
| | TGGCTAAGCTCTGACTGATAAGGAA | |
| | TCATGAATCAAGCACTCGTGATTTT | |
| | GTTGGTATCTTTCCAGCTCGGCGTT | |
| | GCCTTAGATAACTCAGTGTTGGCTC | |
| | CAATAGGAGTAGCTAGCGCACAGGA | |
| | GTGGCAACTGGCGGCATATACAACG | |
| | ACCCTCACAGGGACCATCGCAGTGA | |
| | GATTTATCCCGGTCCTGCCTGGGAA | |
| | CCTATCAACATGTGCACAGGAGACG | |
| | CTGCAGGAATATAATAGAACTGTGA | |
| | CTAATATCTTAGGCCCGTTGAGAGA | |
| | GAACTTGGATGCTCTCCTATCTGAC | |
| | TTCGATAAACCTGCATCGAGGTTCG | |
| | TGGGCGCCATCATTGGGTCGGTGGC | |
| | CTTGGGGGTAGCAACAGCTGCACAA | |
| | ATCACAGCCGCCGTGGCTCTCAATC | |
| | AAGCACAAGAGAATGCCCGGAATAT | |
| | ATGGCGTCTCAAGGAATCGATAAAG | |
| | AAAACCAATGCGGCTGTGTTGGAAT | |
| | TGAAGGATGGACTTGCAACGACTGC | |
| | TATAGCTTTGGACAAAGTGCAAAAG | |
| | TTTATCAATGATGATATTATACCAC | |
| | AGATTAAGGACATTGACTGCCAGGT | |
| | AGTTGCAAATAAATTAGGCGTCTAC | |
| | CTCTCCTTATACTTAACAGAGCTTA | |
| | CAACTGTATTTGGTTCTCAGATCAC | |
| | TAATCCTGCATTATCAACGCTCTCT | |
| | TACCAGGCGCTGTACAGCTTATGTG | |
| | GAGGGGATATGGGAAAGCTAACTGA | |
| | GCTGATCGGTGTCAATGCAAAGGAT | |
| | GTGGGATCCCTCTACGAGGCTAACC | |
| | TCATAACCGGCCAAATCGTTGGATA | |
| | TGACCCTGAACTACAGATAATCCTC | |
| | ATACAAGTATCTTACCCAAGTGTGT | |
| | CTGAAGTGACAGGAGTCCGGGCTAC | |
| | TGAGTTAGTCACTGTCAGTGTCACT | |
| | ACACCAAAAGGAGAAGGGCAGGCAA | |
| | TTGTTCCGAGATATGTGGCACAGAG | |
| | TAGAGTGCTGACAGAGGAGTTGGAT | |
| | GTCTCGACTTGTAGGTTTAGCAAAA | |
| | CAACTCTTTATTGTAGGTCGATTCT | |
| | CACACGGCCCCTACCAACTTTGATC | |
| | GCCAGCTGCCTGTCAGGGAAGTACG | |
| | ACGATTGTCAGTACACAACAGAGAT | |
| | AGGAGCGCTATCTTCGAGATTCATC | |
| | ACAGTCAATGGTGGAGTCCTTGCAA | |
| | ACTGCAGAGCAATTGTGTGTAAGTG | |
| | TGTCTCACCCCCGCATATAATACCA | |
| | CAAAACGACATTGGCTCCGTAACAG | |
| | TTATTGACTCAAGTATATGCAAGGA | |
| | AGTTGTCTTAGAGAGTGTGCAGCTT | |
| | AGGTTAGAAGGAAAGCTGTCATCCC | |
| | AATACTTCTCCAACGTGACAATTGA | |
| | CCTTTCCCAAATCACAACGTCAGGG | |
| | TCGCTGGATATAAGCAGTGAAATTG | |
| | GTAGCATTAACAACACAGTTAATCG | |
| | GGTCGACGAGTTAATCAAGGAATCC | |
| | AACGAGTGGCTGAACGCTGTGAACC | |
| | CCCGCCTTGTGAACAATACGAGCAT | |
| | CATAGTCCTCTGTGTCCTTGCCGCC | |
| | CTGATTATTGTCTGGCTAATAGCGC | |
| | TGACAGTATGCTTCTGTTACTCCGC | |
| | AAGATACTCAGCTAAGTCAAAACAG | |
| | ATGAGGGGCGCTATGACAGGGATCG | |
| | ATAATCCATATGTAATACAGAGTGC | |
| | AACTAAGATGTAGAGAGGTTGAATA | |
| | AGCCTAAACATGATATGATTTAAGA | |
| | AAAAATTGGAAGGTGGGGGCGACAG | |
| | CCCATTCAATGAAGGGTGTACACTC | |
| | CAACTTGATCTTGTGACTTGATCAT | |
| | CATACTCGAGGCACCATGGATTTCC | |
| | CATCTAGGGAGAACCTGGCAGCAGG | |
| | TGACATATCGGGGCGGAAGACTTGG | |
| | AGATTACTGTTCCGGATCCTCACAT | |
| | TGAGCATAGGTGTGGTCTGTCTTGC | |
| | CATCAATATTGCCACAATTGCAAAA | |
| | TTGGATCACCTGGATAACATGGCTT | |
| | CGAACACATGGACAACAACTGAGGC | |
| | TGACCGTGTGATATCTAGCATCACG | |
| | ACTCCGCTCAAAGTCCCTGTCAACC | |
| | AGATTAATGACATGTTTCGGATTGT | |
| | AGCGCTTGACCTACCTCTGCAGATG | |
| | ACATCATTACAGAAAGAAATAACAT | |
| | CCCAAGTCGGGTTCTTGGCTGAAAG | |
| | TATCAACAATGTTTTATCCAAGAAT | |
| | GGATCTGCAGGCCTGGTTCTTGTTA | |
| | ATGACCCTGAATATGCAGGGGGGAT | |
| | CGCTGTCAGCTTGTACCAAGGAGAT | |
| | GCATCTGCAGGCCTAAATTTCCAGC | |
| | CCATTTCTTTAATAGAACATCCAAG | |
| | TTTTGTCCCTGGTCCTACTACTGCT | |
| | AAGGGCTGTATAAGGATCCCGACCT | |
| | TCCATATGGGCCCTTCACATTGGTG | |
| | TTACTCACATAACATCATTGCATCA | |
| | GGTTGCCAGGATGCGAGCCACTCCA | |
| | GTATGTATATCTCTCTGGGGGTGCT | |
| | GAAAGCATCGCAGACCGGGTCGCCT | |
| | ATCTTCTTGACAACGGCCAGCCATC | |
| | TCGTGGATGACAACATCAACCGGAA | |
| | GTCATGCAGCATCGTAGCCTCAAAA | |
| | TACGGTTGTGATATCCTATGCAGTA | |
| | TTGTGATTGAAACAGAGAATGAGGA | |
| | TTATAGGTCTGATCCGGCTACTAGC | |
| | ATGATTATAGGTAGGCTGTTCTTCA | |
| | ACGGGTCATACACAGAGAGCAAGAT | |
| | TAACACAGGGTCCATCTTCAGTCTA | |
| | TTCTCTGCTAACTACCCTGCGGTGG | |
| | GGTCGGGTATTGTAGTCGGGGATGA | |
| | AGCCGCATTCCCAATATATGGTGGG | |
| | GTCAAGCAGAACACATGGTTGTTCA | |
| | ACCAGCTCAAGGATTTTGGTTACTT | |
| | CACCCATAATGATGTGTACAAGTGC | |
| | AATCGGACTGATATACAGCAAACTA | |
| | TCCTGGATGCATACAGGCCACCTAA | |
| | AATCTCAGGAAGGTTATGGGTACAA | |
| | GGCATCCTATTGTGCCCAGTTTCAC | |
| | TGAGACCTGATCCTGGCTGTCGCTT | |
| | AAAGGTGTTCAATACCAGCAATGTG | |
| | ATGATGGGGGCAGAAGCGAGGTTGA | |
| | TCCAAGTAGGCTCAACCGTGTATCT | |
| | ATACCAACGCTCATCCTCATGGTGG | |
| | GTGGTAGGACTGACTTACAAATTAG | |
| | ATGTGTCAGAAATAACTTCACAGAC | |
| | AGGTAACACACTCAACCATGTAGAC | |
| | CCCATTGCCCATACAAAGTTCCCAA | |
| | GACCATCTTTCAGGCGAGATGCGTG | |
| | TGCGAGGCCAAACATATGCCCTGCT | |
| | GTCTGTGTCTCCGGAGTTTATCAGG | |
| | ACATTTGGCCGATCAGTACAGCCAC | |
| | CAATAACAGCAACATTGTGTGGGTT | |
| | GGACAGTACTTAGAAGCATTCTATT | |
| | CCAGGAAAGACCCAAGAATAGGGAT | |
| | AGCAACCCAGTATGAGTGGAAAGTC | |
| | ACCAACCAGCTGTTCAATTCGAATA | |
| | CTGAGGGAGGGTACTCAACCACAAC | |
| | ATGCTTCCGGAACACCAAACGGGAC | |
| | AAGGCATATTGTGTAGTGATATCAG | |
| | AGTACGCTGATGGGGTGTTCGGATC | |
| | ATACAGGATCGTTCCTCAGCTTATA | |
| | GAGATTAGAACAACCACCGGTAAAT | |
| | CTGAGTGATGCATCAATCCTAAATT | |
| | GGAATGACCAATCAAAAGCTACGTA | |
| | GTGTCTAACAGCATTGCGAAGCCTG | |
| | GTTTAAGAAAAAACTTGGGGGCGAA | |
| | TGCCCATCAACCATGGATCAAACTC | |
| | AAGCTGACACTATAATACAACCTGA | |
| | AGTCCATCTGAATTCACCACTTGTT | |
| | CGCGCAAAATTGGTTCTTCTATGGA | |
| | AATTGACTGGGTTACCTTTGCCGTC | |
| | TGATTTGAGATCATTTGTACTAACT | |
| | ACACATGCAGCTGATGACCAAATCG | |
| | CAAAAAATGAGACTAGGATCAAGGC | |
| | CAAAATTAATTCCCTAATCGATAAC | |
| | TTAATCAAACACTGCAAGGCAAGGC | |
| | AAGTGGCACTTTCAGGGTTGACACC | |
| | TGTCGTACATCCAACAACTCTACAG | |
| | TGGTTGCTATCCATCACATGTGAAC | |
| | GAGCAGACCACCTTGCAAAAGTACG | |
| | CGAGAAATCAGTTAAGCAAGCAATG | |
| | TCAGAGAAGCAACACGGGTTTAGAC | |
| | ATCTCTTTTCGGCAGTAAGTCATCA | |
| | GTTAGTTGGAAACGCCACACTGTTC | |
| | TGTGCACAAGACTCTAGCACCGTGA | |
| | ATGTCGACTCTCCTTGCTCATCAGG | |
| | TTGTGAGAGGCTGATAATAGACTCT | |
| | ATTGGAGCCTTACAAACACGATGGA | |
| | CAAGATGTAGGTGGGCTTGGCTTCA | |
| | CATTAAACAGGTAATGAGATACCAG | |
| | GTGCTTCAGAGTCGCCTACACGCTC | |
| | ATGCCAATTCTGTTAGCACATGGTC | |
| | TGAGGCGTGGGGGTTCATTGGGATC | |
| | ACACCAGATATAGTCCTTATTGTAG | |
| | ACTATAAGAGCAAAATGTTTACTAT | |
| | CCTGACCTTCGAAATGATGCTGATG | |
| | TATTCAGATGTCATAGAGGGTCGTG | |
| | ATAATGTGGTAGCTGTAGGAAGTAT | |
| | GTCACCAAACCTACAGCCTGTGGTG | |
| | GAGAGGATTGAGGTGCTGTTTGATG | |
| | TAGTGGACACCTTGGCGAGGAGGAT | |
| | TCATGATCCTATTTATGATCTGGTT | |
| | GCTGCCTTAGAAAGCATGGCATACG | |
| | CTGCCGTCCAATTGCACGATGCTAG | |
| | TGAGACACACGCAGGGGAATTCTTT | |
| | TCGTTCAATTTGACAGAAATAGAGT | |
| | CCACTCTTGCCCCCTTGCTGGATCC | |
| | TGGCCAAGTCCTATCGGTGATGAGG | |
| | ACTATCAGTTATTGTTACAGTGGGC | |
| | TATCGCCTGACCAAGCTGCAGAGTT | |
| | GCTCTGTGTGATGCGCTTATTTGGA | |
| | CACCCTCTGCTCTCCGCACAACAAG | |
| | CAGCCAAAAAAGTCCGGGAGTCTAT | |
| | GTGTGCCCCTAAACTGTTAGAGCAT | |
| | GATGCAATACTGCAAACTCTATCTT | |
| | TCTTCAAGGGAATCATAATCAATGG | |
| | CTACAGGAAAAGTCATTCTGGAGTA | |
| | TGGCCTGCAATTGACCCAGATTCTA | |
| | TAGTGGACGATGACCTTAGACAGCT | |
| | GTATTACGAGTCGGCAGAAATTTCA | |
| | CATGCTTTCATGCTTAAGAAATATC | |
| | GGTACCTTAGTATGATTGAGTTCCG | |
| | CAAGAGCATAGAGTTTGACTTAAAT | |
| | GATGACCTGAGCACATTCCTTAAAG | |
| | ACAAAGCAATCTGCAGGCCAAAAGA | |
| | TCAATGGGCACGCATCTTCCGGAAA | |
| | TCATTGTTCCCTTGCAAAACGAACC | |
| | TTGGCACTAGTATAGATGTTAAAAG | |
| | TAATCGACTGTTGATAGATTTTTTG | |
| | GAGTCACATGACTTCAATCCTGAGG | |
| | AAGAAATGAAGTATGTGACTACGCT | |
| | AGCATACCTGGCAGATAATCAATTC | |
| | TCAGCATCATATTCACTGAAGGAGA | |
| | AAGAGATCAAGACTACTGGCCGGAT | |
| | CTTCGCCAAAATGACCAGGAAAATG | |
| | AGGAGCTGTCAAGTAATATTGGAAT | |
| | CACTATTGTCCAGTCACGTCTGCAA | |
| | ATTCTTTAAGGAGAACGGTGTGTCA | |
| | ATGGAACAACTGTCTTTGACAAAGA | |
| | GCTTGCTTGCAATGTCACAGTTAGC | |
| | ACCCAGGATATCTTCAGTTCGCCAG | |
| | GCGACAGCACGTAGACAGGACCCAG | |
| | GACTCAGCCACTCTAATGGTTGTAA | |
| | TCACATTGTAGGAGACTTAGGCCCA | |
| | CACCAGCAGGACAGACCGGCCCGGA | |
| | AGAGTGTAGTCGCAACCTTCCTTAC | |
| | AACAGATCTTCAAAAATATTGCTTG | |
| | AATTGGCGATATGGGAGTATCAAGC | |
| | TTTTCGCCCAAGCCTTAAACCAGCT | |
| | ATTCGGAATCGAGCATGGGTTTGAA | |
| | TGGATACACCTGAGACTGATGAATA | |
| | GCACCCTGTTTGTCGGGGACCCATT | |
| | CTCGCCTCCTGAAAGCAAAGTGCTG | |
| | AGTGATCTTGATGATGCGCCCAATT | |
| | CAGACATATTTATCGTGTCCGCCAG | |
| | AGGGGGGATTGAAGGGTTATGCCAG | |
| | AAGCTGTGGACCATGATTTCAATAA | |
| | GCATAATCCATTGCGTGGCTGAGAA | |
| | GATAGGAGCAAGGGTTGCGGCGATG | |
| | GTTCAGGGAGATAATCAGGTAATTG | |
| | CAATCACGAGAGAGCTGTATAAGGG | |
| | AGAGACTTACACGCAGATTCAGCCG | |
| | GAGTTAGATCGATTAGGCAATGCAT | |
| | TTTTTGCTGAATTCAAAAGACACAA | |
| | CTATGCAATGGGACATAATCTGAAG | |
| | CCCAAAGAGACAATCCAAAGTCAAT | |
| | CATTCTTTGTGTATTCGAAACGGAT | |
| | TTTCTGGGAAGGGAGAATTCTTAGT | |
| | CAAGCACTGAAGAATGCTACCAAAC | |
| | TATGCTTCATTGCAGATCACCTCGG | |
| | GGATAATACTGTCTCATCATGCAGC | |
| | AATCTAGCCTCTACGATAACCCGCT | |
| | TGGTTGAGAATGGGTATGAAAAGGA | |
| | CACAGCATTCATTCTGAATATCATC | |
| | TCAGCAATGACTCAGTTGCTGATTG | |
| | ATGAGCAATATTCCCTACAAGGAGA | |
| | CTACTCAGCTGTGAGAAAACTGATT | |
| | GGGTCATCAAATTACCGTAATCTCT | |
| | TAGTGGCGTCGCTCATGCCTGGTCA | |
| | GGTTGGCGGCTATAATTTCTTGAAT | |
| | ATCAGTCGCCTATTCACACGCAATA | |
| | TTGGTGATCCAGTAACATGCGCCAT | |
| | AGCAGATCTGAAGTGGTTCATTAGG | |
| | AGCGGGTTAATCCCAGAGTTCATCC | |
| | TGAAGAATATATTACTACGAGATCC | |
| | CGGAGACGATATGTGGAGTACTCTA | |
| | TGTGCTGACCCTTACGCATTAAATA | |
| | TCCCCTACACTCAGCTACCCACAAC | |
| | ATACCTGAAGAAGCATACTCAGAGG | |
| | GCATTACTATCCGATTCTAATAATC | |
| | CGCTTCTTGCAGGGGTGCAATTGGA | |
| | CAATCAATACATTGAAGAGGAGGAG | |
| | TTTGCACGATTCCTTTTGGATCGGG | |
| | AATCCGTGATGCCTCGAGTGGCACA | |
| | CACAATCATGGAGTCAAGTATACTA | |
| | GGGAAGAGAAAGAACATCCAGGGTT | |
| | TAATCGACACTACCCCTACAATCAT | |
| | TAAGACTGCACTCATGAGGCAGCCC | |
| | ATATCTCGTAGAAAGTGTGATAAAA | |
| | TAGTTAATTACTCGATTAACTACCT | |
| | GACTGAGTGCCACGATTCATTATTG | |
| | TCCTGTAGGACATTCGAGCCAAGGA | |
| | AGGAAATAATATGGGAGTCAGCTAT | |
| | GATCTCAGTAGAAACTTGCAGTGTC | |
| | ACAATTGCGGAGTTCCTGCGCGCCA | |
| | CCAGCTGGTCCAACATCCTGAACGG | |
| | TAGGACTATTTCGGGTGTAACATCT | |
| | CCAGACACTATAGAGCTGCTCAAGG | |
| | GGTCATTAATTGGAGAGAATGCCCA | |
| | TTGTATTCTTTGTGAGCAGGGAGAC | |
| | GAGACATTCACGTGGATGCACTTAG | |
| | CCGGGCCCATCTATATACCAGACCC | |
| | GGGGGTGACCGCATCCAAGATGAGA | |
| | GTGCCGTATCTTGGGTCAAAGACAG | |
| | AGGAAAGGCGTACGGCATCCATGGC | |
| | CACCATTAAGGGCATGTCTCACCAC | |
| | CTAAAGGCCGCTTTGCGAGGAGCCT | |
| | CTGTGATGGTGTGGGCCTTTGGTGA | |
| | TACTGAAGAAAGTTGGGAACATGCC | |
| | TGCCTTGTGGCCAATACAAGGTGCA | |
| | AGATTAATCTTCCGCAGCTACGCCT | |
| | GCTGACCCCGACACCAAGCAGCTCT | |
| | AACATCCAACATCGACTAAATGATG | |
| | GTATCAGCGTGCAAAAATTTACACC | |
| | TGCTAGCTTATCCCGAGTGGCGTCA | |
| | TTTGTTCACATTTGCAACGATTTCC | |
| | AAAAGCTAGAGAGAGATGGATCTTC | |
| | CGTAGACTCTAACTTGATATATCAG | |
| | CAAATCATGCTGACTGGTCTAAGTA | |
| | TTATGGAGACACTTCATCCTATGCA | |
| | CGTCTCATGGGTATACAACAATCAG | |
| | ACAATTCACTTACATACCGGAACAT | |
| | CGTGTTGTCCTAGGGAAATAGAGAC | |
| | AAGCATTGTTAATCCCGCTAGGGGA | |
| | GAATTCCCAACAATAACTCTCACAA | |
| | CTAACAATCAGTTTCTGTTTGATTG | |
| | TAATCCCATACATGATGAGGCACTT | |
| | ACAAAACTGTCAGTAAGTGAGTTCA | |
| | AGTTCCAGGAGCTTAATATAGACTC | |
| | AATGCAGGGTTACAGTGCTGTGAAC | |
| | CTGCTGAGCAGATGTGTGGCTAAGC | |
| | TGATAGGGGAATGCATTCTGGAAGA | |
| | CGGTATCGGATCGTCAATCAAGAAT | |
| | GAAGCAATGATATCATTTGATAACT | |
| | CTATCAACTGGATTTCTGAAGCACT | |
| | CAATAGTGACCTGCGTTTGGTATTC | |
| | CTCCAGCTGGGGCAAGAACTACTTT | |
| | GTGACCTGGCGTACCAAATGTACTA | |
| | TCTGAGGGTCATCGGCTATCATTCC | |
| | ATCGTGGCATATCTGCAGAATACTC | |
| | TAGAAAGAATTCCTGTTATCCAACT | |
| | CGCAAACATGGCACTCACCATATCC | |
| | CACCCAGAAGTATGGAGGAGAGTGA | |
| | CAGTGAGCGGATTCAACCAAGGTTA | |
| | CCGGAGTCCCTATCTGGCCACTGTC | |
| | GACTTTATCGCCGCATGTCGTGATA | |
| | TCATTGTGCAAGGTGCCCAGCATTA | |
| | TATGGCTGATTTGTTGTCAGGAGTA | |
| | GAGTGCCAATATACATTCTTTAATG | |
| | TTCAAGACGGCGATCTGACACCGAA | |
| | GATGGAACAATTTTTAGCCCGGCGC | |
| | ATGTGCTTGTTTGTATTGTTAACTG | |
| | GGACGATCCGACCACTCCCAATCAT | |
| | ACGATCCCTTAATGCGATTGAGAAA | |
| | TGTGCAATTCTCACTCAGTTCTTGT | |
| | ATTACCTACCGTCAGTCGACATGGC | |
| | AGTAGCAGACAAGGCTCGTGTGTTA | |
| | TATCAACTGTCAATAAATCCGAAAA | |
| | TAGATGCTTTAGTCTCCAACCTTTA | |
| | TTTCACCACAAGGAGGTTGCTTTCA | |
| | AATATCAGGGGAGATTCTTCTTCAC | |
| | GAGCGCAAATTGCATTCCTCTACGA | |
| | GGAGGAAGTAATCGTTGATGTGCCT | |
| | GCATCTAATCAATTTGATCAGTACC | |
| | ATCGTGACCCCATCCTAAGAGGAGG | |
| | TCTATTTTTCTCTCTCTCCTTAAAA | |
| | ATGGAAAGGATGTCTCTGAACCGAT | |
| | TTGCAGTACAGACCCTGCCAACCCA | |
| | GGGGTCTAACTCGCAGGGTTCACGA | |
| | CAGACCTTGTGGCGTGCCTCACCGT | |
| | TAGCACACTGCCTTAAATCAGTAGG | |
| | GCAGGTAAGTACCAGCTGGTACAAG | |
| | TATGCTGTAGTGGGGGCGTCTGTAG | |
| | AGAAAGTCCAACCAACAAGATCAAC | |
| | AAGCCTCTACATCGGGGAGGGCAGT | |
| | GGGAGTGTCATGACATTATTAGAGT | |
| | ATCTGGACCCTGCTACAATTATCTT | |
| | CTACAACTCGCTATTCAGCAATAGC | |
| | ATGAACCCTCCACAAAGGAATTTCG | |
| | GACTGATGCCCACACAGTTTCAGGA | |
| | CTCAGTCGTGTATAAAAACATATCA | |
| | GCAGGAGTTGACTGCAAGTACGGGT | |
| | TTAAGCAAGTCTTTCAACCATTATG | |
| | GCGTGATGTAGATCAAGAAACAAAT | |
| | GTGGTAGAGACGGCGTTCCTAAACT | |
| | ATGTGATGGAAGTAGTGCCAGTCCA | |
| | CTCTTCGAAGCGTGTCGTATGTGAA | |
| | GTTGAGTTTGACAGGGGGATGCCTG | |
| | ACGAGATAGTAATAACAGGGTACAT | |
| | ACACGTGCTGATGGTGACCGCATAC | |
| | AGTCTGCATCGAGGAGGGCGTCTAA | |
| | TAATCAAGGTCTATCGTCACTCCGA | |
| | GGCTGTATTCCAATTCGTACTCTCT | |
| | GCGATAGTCATGATGTTTGGGGGGC | |
| | TTGATATACACCGGAACTCGTACAT | |
| | GTCAACTAACAAAGAGGAGTACATC | |
| | ATCATAGCTGCGGCGCCGGAGGCAT | |
| | TAAACTATTCCTCTGTACCAGCAAT | |
| | ATTGCAGAGGGTGAAGTCTGTTATT | |
| | GACCAGCAGCTTACATTAATCTCTC | |
| | CTATAGATCTAGAAAGATTGCGCCA | |
| | TGAGACTGAGTCTCTCCGTGAGAAG | |
| | GAGAATAATCTAGTAATATCTCTGA | |
| | CGAGAGGGAAGTATCAACTCCGGCC | |
| | GACACAGACTGATATGCTTCTATCA | |
| | TACCTAGGTGGGAGATTCATCACCC | |
| | TATTCGGACAGTCTGCTAGGGATTT | |
| | GATGGCCACTGATGTTGCTGACCTT | |
| | GATGCTAGGAAGATTGCATTAGTTG | |
| | ATCTACTGATGGTGGAATCCAACAT | |
| | TATTTTAAGTGAGAGCACAGACTTG | |
| | GACCTTGCACTGTTGCTGAGCCCGT | |
| | TTAACTTAGACAAAGGGCGGAAGAT | |
| | AGTTACCCTAGCAAAGGCTACTACC | |
| | CGCCAATTGCTGCCCGTGTATATCG | |
| | CATCAGAGATAATGTGCAATCGGCA | |
| | GGCATTCACACACCTGACATCAATT | |
| | ATACAGCGTGGTGTCATAAGAATAG | |
| | AAAACATGCTTGCTACAACGGAATT | |
| | TGTCCGACAGTCAGTTCGCCCCCAG | |
| | TTCATAAAGGAGGTGATAACTATAG | |
| | CCCAAGTCAACCACCTTTTTTCAGA | |
| | TCTATCCAAACTCGTGCTTTCTCGA | |
| | TCTGAAGTCAAGCAAGCACTTAAAT | |
| | TTGTCGGTTGCTGTATGAAGTTCAG | |
| | AAATGCAAGCAATTAAACAGGATTG | |
| | TTATTGTCAAATCACCGGTTACTAT | |
| | AGTCAAATTAATATGTAAAGTTCCC | |
| | TCTTTCAAGAGTGATTAAGAAAAAA | |
| | CGCGTCAAAGGTGGCGGTTTCACTG | |
| | ATTTGCTCTTGGAAGTTGGGCATCC | |
| | TCCAGCCAATATATCGGTGCCGAAA | |
| | TCGAAAGTCTGACAGCTGATTTGGA | |
| | ATATAAGCACTGCATAATCACTGAG | |
| | TTACGTTGCTTTGCTATTCCATGTC | |
| | TGGT | |
| Avian | ACTAAACAGAAAGTTAATAAGTGTT | SEQ ID |
| paramyxovir | TGTAACGTCCGATTAAGTAGCCAGA | NO: 2 |
| us 3 strain | TTAATAGGAGCGGAAGTCCTAAATT | |
| turkey/ | CCGCGTCCGACTGCGAATTTCAATA | |
| Wisconsin/68, | ACTATGGCAGGTATCTTCAATACAT | |
| complete | ATGAGTTGTTCGTCAAGGACCAAAC | |
| genome | ATGCATGCACAAGCGGGCAGCAAGT | |
| Genbank: | CTCATATCAGGGGGGCAGCTCAAAA | |
| EU782025.1 | GCAACATCCCAGTATTCATTACCAC | |
| | CAGGGATGACCCGGCCGTGAGGTGG | |
| | AATCTTGTTTGCTTTAATCTAAGGT | |
| | TAATTGTCAGTGAGTCCTCAACATC | |
| | AGTTATTCGCCAAGGAGCAATGATC | |
| | TCACTTTTGTCAGTCACAGCAAGTA | |
| | ACATGAGGGCTTTAGCAGCAATCGC | |
| | TGGTCAGACAGATGAGTCAATGATT | |
| | AATATAATTGAAGTTGTTGATTTCA | |
| | ATGGGTTAGAGCCACAATGTGATCC | |
| | AAGGAGTGGCCTTGATGCTCAGAAG | |
| | CAAGACATGTTTAAAGACATTGCAA | |
| | GTGATATGCCGAAGGTTCTCGGAAG | |
| | TGGCACACCTTTCCAGAATGTAAGT | |
| | GCAGAGACCAACAATCCAGAGGATA | |
| | CACACATGTTCTTACGCTCAGCAAT | |
| | CAGCGTCCTGACTCAAATCTGGATT | |
| | TTGGTAGCAAAAGCCATGACTAATA | |
| | TCGAAGGTAGTCATGAGGCCAGTGA | |
| | TAGAAGGCTTGCGAAATACACCCAG | |
| | CAGAACAGAATTGACCGGCGCTTTA | |
| | TGCTGGCCCAAGCCACTCGGACTGC | |
| | ATGCCAGCAAATAATAAAGGACTCA | |
| | CTAACAATTAGAAGGTTTCTGGTCA | |
| | CGGAACTTCGGAAGTCGCGAGGGGC | |
| | TCTTCATAGTGGGTCATCATATTAT | |
| | GCAATGGTAGGAGATATGCAAGCAT | |
| | ACATCTTTAATGCTGGACTTACTCC | |
| | TTTCCTCACAACACTCAGGTATGGT | |
| | ATTGGTACCAAATACCACGCTCTCG | |
| | CAATCAGTTCTCTGACGGGAGACCT | |
| | TAATAAGATTAAGGGATTGCTAACA | |
| | CTGTACAAGGAAAAGGGGAGTGACG | |
| | CAGGGTATATGGCATTATTAGAGGA | |
| | TGCAGATTGCATGCAATTTGCACCA | |
| | GGGAACTATGCGTTGCTGTACTCGT | |
| | ATGCAATGGGAGTTGCCAGTGTCCA | |
| | TGATGAAGGCATGAGAAACTACCAG | |
| | TATGCAAGGCGGTTTCTGCACAAAG | |
| | GCATGTACCAGTTTGGAAGAGACAT | |
| | TGCAACACAACACCAGCATGCATTG | |
| | GATGAGTCTCTTGCTCAGGAAATGA | |
| | GAATCACCGAGGCGGACCGGGCCAA | |
| | TCTCAAAGTAATGATGGCAAATATC | |
| | GGTGAGGCTTCCCATTACAGTGATA | |
| | TTCCCAGTGCGGGCCCCAGTGGCAT | |
| | ACCAGCATTTAACGATCCACCAGAA | |
| | GAGTTATTTGGAGAGCCCTCATACA | |
| | GGAAGTTGCCCGAAGAGCCTCAAGT | |
| | TGTAGAACTACAAGACCGGGATGAC | |
| | GATGAGCAAGATGAATATGATATGT | |
| | AATCCTTCAGGAGAACACCCCCACC | |
| | ACCCAACAGCCCCCGAAAATTAAAA | |
| | ACACTCCCTCCCCGACAACCCGCAC | |
| | ACCCCACGGCCATCACCCCCCCATC | |
| | AGCACCCAATCCCAAGCGCAGACAG | |
| | GCCACCGCCTCCACCCAGAACCCCA | |
| | GGACCCAAATCCCCACTATATCTTT | |
| | AAGAAAAAAAGACCTGATGTGTACG | |
| | AGGAGAAAAATAATTGATGACAAGC | |
| | GGAGAAAATAGGAGCGGAAGTATCC | |
| | CTCCTAACAAGATAGACACAATTAT | |
| | CATGGATCTTGAATTCAGCAGTGAG | |
| | GAGGCAGTTGCAGCTTTGCTCGACG | |
| | TGAGTTCATCCACTATCACAGAGTT | |
| | CCTAAGCAAACAAAGCATCCCCGAT | |
| | CCGGGATTCCTAAATTCACCTTCCC | |
| | AGTCAAGCAGTCCCTCCCCTGAACC | |
| | AAGCACCTCTACTACCGGTGACTTC | |
| | CTCTCACAGCTATCAGGTGATATCC | |
| | CTGATACCACCACATCAGGTGTAGA | |
| | ACCATCAGCACCTCTAGATACAGGT | |
| | GACACCTCGTTGGTACAACATATTG | |
| | AGGAGGGACTGCCCTCAGACTTCTA | |
| | CATACCCAAAGTCAACAACTATCAT | |
| | TCGAACCTTTTTAAAGGGGGCTCCT | |
| | CCCTGCTCGCAACGGCGGAATCCCC | |
| | TGGTCTGACAGTGACCCACAAAGAT | |
| | ACGACTACACCGGAGTCCACACCGG | |
| | TTATGGCGAAGAAGAAGAAGAAGCA | |
| | GAAGCACTGCAAAGTGCCCGCATCT | |
| | TCGGCGTACCAACACATAGACAATC | |
| | TGGGCACCGGAGAGAGTACTCCATT | |
| | GCATGGGATGCAAGATCAGGAACCT | |
| | TCCAAACCGAAACATGGTGTAACCC | |
| | CGCATGTTCCCCAGTCACAGCCCTC | |
| | CCAAAGCAGTATAGATGTGCTTGCC | |
| | GACAATGTCCCAAATTCTGTGACCT | |
| | CTGTTTCAATCCCGCTGACTATGGT | |
| | GGAATCATTGATCTCGCAAGTGTCA | |
| | AAGTTATCGGACCAAGTCTCTCAGA | |
| | TCCAGAAATTGGTGAGCACACTTCC | |
| | CCAAATTAAGACCGACATAGCATCA | |
| | ATCAGGAACATGCAGGCGGCCCTAG | |
| | AAGGTCAAATTAGTATGATAAGGAT | |
| | ACTCGACCCCGGCAACAACACAGAG | |
| | TCATCCCTAAATACCCTCCGCAACT | |
| | CTGGAAATCGGGCTCCAGTAGTGAT | |
| | TTGCGGACCGGGCGACCCTCACCGC | |
| | AGTCTGATCAAAAGCGAGAACCCGA | |
| | CTATCTGCCTGGATGAACTAGCTCG | |
| | GCCAACTCAAGCCAACAGTCCTCCA | |
| | AAATCTCAAGATAACCAAAGGGATC | |
| | TATCCGCTCAACGACACGCAATCAC | |
| | AGCTCTGCTAGAAACCCGCGTTGCA | |
| | CCCGGACCTAAGAGAGATCGCCTGA | |
| | TGGAAATGGTAGTAGCAGCGAAATC | |
| | AGCAAGTGATCTCATCAAAGTCAAG | |
| | AGAATGGCAATTCTTGGTCAATAAA | |
| | CCGACTCAGCACCACATTGTCTGTG | |
| | ACTCTACACTTGTGCGGCAAACCAA | |
| | CATTGACCTCCAAACACTTTTCTGC | |
| | AGTACGCAAGGCTTAACACAATCAG | |
| | CAGCATGCATATCGAGCGGCCCACC | |
| | CTCACAACCCATCTAGCTCTCTTAT | |
| | TTTATCTATTGCTTTATAAAAAACC | |
| | AAAATGATTATAACTAAACAATCTC | |
| | AACAATTTGCAATGATAACAACACC | |
| | ATACGATCACTAGGGGCGGAAGCCC | |
| | AAAATAACCCAAGGACCAATCTCCG | |
| | AGTCCAGGCCAGACACAGGCAACCC | |
| | ATCAGCACAGAGCCAAGCAACCAAA | |
| | ATGGCAGCACACCCCAACCATGCCA | |
| | ACCCATCCTCGTCAATCAGCCTCAT | |
| | GCATGATGATCCATCCATCCAGACG | |
| | CAACTTCTTGCCTTTCCGCTGATCA | |
| | GTGAAAAGACCGAGACGGGCACTAC | |
| | CAAACTTCAACCTCAAGTCAGAATG | |
| | CAGTCATTTCTCTCAACTGACAGCC | |
| | AAAAGTACCACCTGGTATTCATAAA | |
| | TACGTATGGTTTCATAGCCGAGGAC | |
| | TTCAACTGTAGTCCTACCAATGGAT | |
| | TCGTTCCTGCGTTGTTTCAACCGAA | |
| | ATCTAAGGTATTGTCTTCAGCAATG | |
| | GTTACCCTTGGTGCAGTTCCTGCAG | |
| | ATACAGTCCTGCAGGACTTACAAAA | |
| | AGACCTTATAGCCATGCGATTTAAG | |
| | GTCAGGAAGAGTGCATCTGCTAAAG | |
| | AACTCATACTATTCTCTACTGATAA | |
| | TATTCCAGCAACACTTACAGGATCA | |
| | TCTGTTTGGAAAAACAGGGGTGTTA | |
| | TTGCAGACACCGCCACATCCGTGAA | |
| | GGCCCCCGGCAGAATCTCCTGTGAT | |
| | GCAGTCTGCAGTTATTGCATTACTT | |
| | TCATATCATTCTGTTTCTTCCACTC | |
| | ATCTGCCTTATTCAAGGTGCCCAAG | |
| | CCACTGCTTAATTTTGAGACAGCCG | |
| | TTGCCTATTCTCTAGTCCTGCAGGT | |
| | TGAATTGGAATTCCCGAACATAAAG | |
| | GACACCCTACATGAGAAATATTTAA | |
| | AGAACAAGGACTCTAAATGGTACTG | |
| | TACCATTGACATACACATAGGGAAC | |
| | CTCCTGAAAAGGACTGCAAAACAGA | |
| | GAAGGCGTACACCATCTGAAATCAC | |
| | TCAAAAGGTGCGCAGAATGGGCTTT | |
| | CGGATTGGACTCTACGATCTTTGGG | |
| | GCCCTACAATAGTGGTCGAATTAAC | |
| | TGGCTCATCGAGCAAATCGCTCCAG | |
| | GGATTCTTCTCCAGTGAGAGACTGG | |
| | CTTGCCATCCTATTTCACAATACAA | |
| | CCCACATGTCGGTCAACTGATTTGG | |
| | GCACATGATGTTTCAATAACAGGCT | |
| | GTCATATGATAATATCTGAACTTGA | |
| | GAAAAAGAAAGCTTTGGCCATGGCT | |
| | GACCTCACTGTAAGTGATGCAGTTG | |
| | CTATCAATACTACAATAAAGGAGTT | |
| | GGTTCCTTTCCGCTTGTTCAGGAAA | |
| | TAAATCACTCACTGCCGCCAGCTTA | |
| | CCACTAGTAACAAATTACAACCATC | |
| | ACCTATAACCTAACAAACCAAATGC | |
| | ATGCACCTAACCTTCTGGGTTGAAT | |
| | GAGAAGCTTGGATTATATTCATGAT | |
| | TAGCTAACACGAATTTATTGCTTAA | |
| | ATTGCTTATACCGGTAATAACTCAA | |
| | ATATTCCACTAACCAAATTTAATTA | |
| | AAAATATTAATAATCATTAGCAACA | |
| | TCCGATCGGAATCTTCAGGGGCGGA | |
| | AGGACCACCGCCACAACACCCCACC | |
| | ACACCAGACCTCCCCGCGCCCCCAC | |
| | AAGACCGGCCACACCAAACAAAAAG | |
| | CCCCCCCAACCCCCCACACCCTCCC | |
| | CGACAGCCCGACAAAAAACCCCCCC | |
| | AAAAAACAGATCGCCCACACACAGA | |
| | TCAGAATGGCCTCCCCAATGGTCCC | |
| | ACTACTCATCATAACGGTAGTACCC | |
| | GCACTCATTTCAAGTCAATCAGCTA | |
| | ATATTGATAAGCTCATTCAAGCAGG | |
| | GATTATCATGGGCTCAGGGAAGGAA | |
| | CTCCACATTTATCAAGAATCTGGCT | |
| | CTCTTGATTTGTATCTTAGACTATT | |
| | GCCAGTTATCCCTTCAAATCTTTCT | |
| | CATTGCCAGAGTGAAGTAATAACAC | |
| | AATATAACTCGACTGTAACGAGACT | |
| | ATTATCACCAATTGCAAAAAATCTA | |
| | AACCATTTGCTACAACCGAGACCGT | |
| | CTGGCAGGTTATTTGGCGCTGTAAT | |
| | TGGATCGATTGCCTTAGGGGTAGCT | |
| | ACATCCGCACAGATTTCAGCTGCTA | |
| | TAGCATTGGTCCGTGCTCAACAGAA | |
| | TGCAAACGATATCCTCGCTCTTAAA | |
| | GCTGCAATACAATCTAGTAATGAGG | |
| | CAATAAAACAACTTACTTATGGCCA | |
| | AGAAAAGCAACTACTAGCAATATCA | |
| | AAAATACAAAAAGCCGTAAATGAAC | |
| | AAGTAATCCCTGCATTGACTGCACT | |
| | TGACTGTGCAGTTCTTGGAAATAAA | |
| | CTAGCTGCACAACTGAACCTCTACC | |
| | TCATTGAAATGACGACTATTTTTGG | |
| | TGACCAAATAAATAACCCAGTCCTA | |
| | ACTCCAATACCACTCAGTTATCTCC | |
| | TGCGGTTGACAGGCTCTGAGTTAAA | |
| | TGATGTATTATTACAACAGACTCGA | |
| | TCCTCTTTGAGCCTAATCCACCTTG | |
| | TCTCTAAAGGCTTATTAAGTGGTCA | |
| | GATTATAGGATATGACCCTTCAGTA | |
| | CAAGGCATCATTATCAGAATAGGAC | |
| | TGATCAGGACTCAAAGAATAGATCG | |
| | GTCACTAGTTTTCCWACCTTACGTA | |
| | TTACCAATTACTATTAGTTCTAACA | |
| | TAGCCACACCAATTATACCCGACTG | |
| | TGTGGTCAAGAAGGGAGTAATAATT | |
| | GAGGGAATGCTTAAGAGTAATTGTA | |
| | TAGAATTGGAACGAGATATAATTTG | |
| | CAAGACTATCAACACATACCAAATA | |
| | ACTAAGGAAACTAGAGCATGCTTAC | |
| | AAGGTAATATAACAATGTGTAAGTA | |
| | CCAGCAGTCCAGGACACAGTTGAGC | |
| | ACCCCCTTTATTACATATAATGGAG | |
| | TTGTAATTGCAAATTGTGATTTGGT | |
| | ATCATGCCGATGCATAAGACCCCCT | |
| | ATGATTATCACACAAGTAAAAGGTT | |
| | ACCCTCTGACAATTATAAATAGGAA | |
| | TTTATGTACCGAGTTGTCGGTGGAT | |
| | AATTTAATTTTAAATATTGAAACAA | |
| | ACCATAACTTTTCATTAAACCCTAC | |
| | TATTATAGATTCACAATCCCGGCTT | |
| | ATAGCTACTAGTCCATTAGAAATAG | |
| | ATGCCCTTATTCAAGATGCGCAACA | |
| | TCACGCGGCTGCGGCCCTTCTTAAA | |
| | GTAGAAGAAAGCAATGCTCACTTAT | |
| | TAAGAGTTACAGGGCTGGGCTCATC | |
| | AAGTTGGCACATCATACTTATATTA | |
| | ACATTGCTTGTATGCACCATAGCAT | |
| | GGCTCATTGGTTTATCTATTTATGT | |
| | CTGCCGCATTAAAAATGATGACTCG | |
| | ACCGACAAAGAACCTACAACCCAAT | |
| | CATCGAACCGCGGCATTGGGGTTGG | |
| | ATCTATACAATATATGACATAATGA | |
| | GCCGCCTGTATATCAAGCCCAAGTA | |
| | TCGACCCCTCCCACCATCCTCGACC | |
| | GCCGCCACTAGCAGCACAGGAAGTA | |
| | ATCAGTTACAGTGGCATCAGCAGTC | |
| | CCATGTTGAGACACACCAGTACACC | |
| | CTAGTTTCTAGTAAAACCCCCAGTT | |
| | CTATTTTCTGCATTCCATTAATTTA | |
| | TAAAAAAATGCCATGATACTCGTGC | |
| | GAGTGTAACATAGTAACTAGGGGCG | |
| | GAAGCCTACCGCCAAATCAGCACAC | |
| | ACCCCCCCAACATGGAGCCGACAGG | |
| | ATCAAAAGTTGACATTGTCCCTTCC | |
| | CAAGGTACCAAGAGAACATGTCGAA | |
| | CCTTTTATCGCCTCTTAATTCTTAT | |
| | TTTGAATCTTATTATAATTATATTA | |
| | ACAATTATCAGTATTTATGTCTCTA | |
| | TCTCAACAGATCAACACAAATTGTG | |
| | CAATAATGAGGCTGACTCACTTTTA | |
| | CACTCAATAGTAGAACCCATAACAG | |
| | TCCCCCTAGGAACAGACTCGGATGT | |
| | TGAGGATGAATTACGTGAGATTCGA | |
| | CGTGATACAGGCATAAATATTCCTA | |
| | TCCAAATTGACAACACAGAGAACAT | |
| | CATATTAACTACATTAGCAAGTATC | |
| | AACTCTAACATTGCACGCCTTCATA | |
| | ACGCCACCGATGAAAGCCCAACATG | |
| | CCTGTCACCAGTTAATGATCCCAGG | |
| | TTTATAGCAGGGATTAATAAGATAA | |
| | CCAAAGGGTCGATGATATATAGGAA | |
| | TTTCAGCAATTTGATAGAACATGTT | |
| | AACTTTATACCATCTCCAACGACAT | |
| | TATCAGGCTGTACAAGAATTCCATC | |
| | TTTTTCACTATCTAAAACACATTGG | |
| | TGTTACTCGCATAATGTAATATCTA | |
| | CTGGTTGTCAAGACCATGCTGCGAG | |
| | TTCACAGTATATTTCCATAGGAATA | |
| | GTAGATACAGGATTGAATAATGAGC | |
| | CCTATTTGCGTACAATGTCTTCACG | |
| | CTTGCTAAATGATGGCCTAAATAGA | |
| | AAGAGCTGCTCTGTCACAGCCGGCG | |
| | CTGGTGTCTGTTGGCTATTGTGTAG | |
| | TGTTGTAACAGAAAGTGAATCAGCT | |
| | GACTACAGATCAAGAGCCCCCACTG | |
| | CAATGATTCTCGGAAGGTTCAATTT | |
| | TTATGGTGATTACACTGAATCCCCT | |
| | GTTCCTGCATCTTTGTTCAGCGGTC | |
| | GTTTCACTGCTAATTACCCTGGAGT | |
| | TGGCTCAGGAACCCAATTAAATGGG | |
| | ACCCTTTATTTTCCAATATATGGGG | |
| | GTGTTGTTAACGACTCTGATATTGA | |
| | GTTATCGAACCGAGGGAAGTCATTC | |
| | AGACCTAGGAACCCTACAAACCCAT | |
| | GTCCAGATCCTGAGGTGACCCAAAG | |
| | TCAGAGGGCTCAGGCAAGTTACTAT | |
| | CCGACAAGGTTTGGCAGGCTGCTCA | |
| | TACAACAAGCAATACTAGCTTGTCG | |
| | TATTAGTGACACTACATGCACTGAT | |
| | TATTATCTTCTATACTTTGATAATA | |
| | ATCAAGTCATGATGGGTGCAGAAGC | |
| | CCGAATTTATTATTTAAACAATCAG | |
| | ATGTACTTATATCAAAGATCTTCGA | |
| | GTTGGTGGCCGCATCCGCTTTTTTA | |
| | CAGATTCTCACTGCCTCATTGTGAA | |
| | CCTATGTCTGTCTGTATGATCACCG | |
| | ATACACACTTAATATTGACATATGC | |
| | TACCTCACGCCCTGGCACTTCAATT | |
| | TGTACAGGGGCCTCGCGATGTCCTA | |
| | ATAACTGTGTTGATGGTGTCTATAC | |
| | AGACGTTTGGCCCTTGACTGAGGGT | |
| | ACAACACAAGATCCAGATTCCTACT | |
| | ACACAGTATTCCTCAACAGTCCCAA | |
| | CCGCAGGATCAGTCCTACAATTAGC | |
| | ATTTACAGCTACAACCAGAAGATTA | |
| | GCTCTCGTCTGGCTGTAGGAAGTGA | |
| | AATAGGAGCTGCTTACACGACCAGT | |
| | ACATGTTTTAGCAGGACAGACACTG | |
| | GGGCACTATACTGCATCACTATAAT | |
| | AGAAGCTGTAAACACAATCTTTGGA | |
| | CAATACCGAATAGTACCGATCCTTG | |
| | TTCAACTAATTAGTGACTAGGAAAT | |
| | GATGTTTAATTACTCGATGTTGAGT | |
| | AAATGATCCTAGAACTTCTCCTTAG | |
| | AATGATATACATCGCTTGTACTATA | |
| | ATCAAGTAACGGGCAGCGGGTGATC | |
| | CATATTAAATAATATATGCATTAAG | |
| | CAGATACAAATCTTCACTTTGTCAA | |
| | TCAGAATTGATTATTGCACCTTTGC | |
| | CACGTAGATAACTAAGCATTTAAGA | |
| | AAAAACTTCACTATCACTCTTTGAG | |
| | TCGCTGAAGTGAGATTTCAGAAAGG | |
| | TATGCATCTAAGAAGTAGGAGCGGA | |
| | AGTGCTCTTGTTCATAATGTCTTCC | |
| | CACAATATTATCTTACCTGACCATC | |
| | ACTTAAATTCTCCTATAGTACTAAA | |
| | TAAATTAATGTATTACTGCAAATTG | |
| | CTCAATGTATTGCCTGGGCCTGATT | |
| | CTCCTTGGTTTGAGAAAACAAGAGG | |
| | ATGGACTAATTGCTGTATCCGTCTT | |
| | TCTGACTGCAACCGCTTAACTCTAG | |
| | CACGCGCCTCAAGAATTAGAGATCA | |
| | ATTAGCAACAATGGGAATATATTCA | |
| | AAGAATCAATCAACATGTTTTAAAA | |
| | CAATTATTCATCCACAATCCTTGCA | |
| | ACCAATTATGCATAGTGCATCAGAA | |
| | TTAGGACGGACTCTACCTACATGGT | |
| | CGCGAATGAGAAGCGAGGTGTCATA | |
| | CAGTGTAACAACACAATCAGCAAAA | |
| | TTTGGAGACCTATTCCAAGGCATAT | |
| | CTACTGATCTAACAGGGAAGACAAA | |
| | TTTGTTTGGCGGATTCTGCGATTTA | |
| | AATCACTCCCTTAGCCCACCTGCAC | |
| | ATGCATTAATGACTAAGCCTGGGAT | |
| | GTATCTAGAGACTAGTGATGCTTAC | |
| | GCTTGCCAATTTTTGTTCCACATTA | |
| | AAACTTGTCAACGAGAGTTGATCTT | |
| | ACTCATGAGGCAAAATGCAACAGCC | |
| | GAACTGATTAAGCAATTCCAGTATC | |
| | CAGGATTGACAATTATAACCACACC | |
| | TGAATATTCAGTTTGGGTCTTCCAT | |
| | GAAAGCAAACAAGTCACTATCCTTA | |
| | CTTTTGATTGCCTTTTAATGTACTG | |
| | TGATCTCGCTGATGGGCGTCACAAT | |
| | ATCCTCTTTACATGCCAATTACTTC | |
| | CGCACTTAAATCATCTAGGTATAAG | |
| | GATCCGAGACCTCTTAGGGCTAATA | |
| | GATAATCTCGGGAAGAATCATCCCT | |
| | TGATTGTGTATGATGTTGTTGCTAG | |
| | TTTAGAATCATTGGCATATGGGGCC | |
| | ATACAACTCCATGACAAAGTTGTTG | |
| | ATTATGCAGGTACCTTCTTCACTTT | |
| | CATTCTGGCTGAGATATATGAATCT | |
| | TTAGAGTCCTCTCTACCAAGTGGAA | |
| | ATAGTGAAGCGATTGTTACTCAAAT | |
| | TAGGAACATATATACAGGGTTAACA | |
| | GTAAATGAAGCAGCTGAGCTCTTAT | |
| | GTGTAATGAGACTCTGGGGGCATCC | |
| | TGCATTAAGCAGTATAGATGCAGCA | |
| | AATAAGGTGCGGCAAAGTATGTGCG | |
| | CAGGGAAACTGTTAAAATTTGATAC | |
| | GATCCAACTGGTATTAGCCTTCTTC | |
| | AATACGTTAATTATCAATGGCTATC | |
| | GCAGGAAACATCATGGTAGGTGGCC | |
| | AAATGTGGATAGTAATTCAATCTTA | |
| | GGAACAGATCTTAAGAGGATGTATT | |
| | ATGATCAATGTGAAATCCCCCATGA | |
| | GTTTACACTTAAACATTATCATACT | |
| | GTGAGTCTAATTGAGTTTGATTGTA | |
| | CGTTTCCAATCGAGCTATCCGACAA | |
| | ATTAAACATATTTCTTAAAGATAAG | |
| | GCAATTGCATTCCCTAAGTCAAAGT | |
| | GGACATCTCCTTTTAAAGCCGATAT | |
| | CACACCTAAACAATTACTCATCCCT | |
| | CCCGAATTTAAAGTTCGTGCAAATC | |
| | GCCTTCTCTTGACTTTCCTGCAGTT | |
| | AGATGAGTTTTCTATCGAATCAGAA | |
| | TTAGAATATGTTACAACCAAAGCAT | |
| | ATCTCGAAGATGATGAGTTCAATGT | |
| | ATCATACTCTCTCAAGGAGAAAGAA | |
| | GTGAAGACAGATGGTCGCATATTTG | |
| | CTAAATTAACTCGTAAGATGAGGAG | |
| | TTGTCAAGTAATCTTTGAAGAGCTC | |
| | CTTGCCGAACATGTGTCCCCCCTTT | |
| | TCAAAGACAACGGTGTAACTATGGC | |
| | TGAATTATCATTGACCAAAAGCCTA | |
| | CTTGCAATAAGCAATTTAAGTTCCA | |
| | CATTGTTTGAGACACAAACCCGTCA | |
| | GGGCGACAGAAATTCAAGATTTACT | |
| | CATGCTCATTTTATTACAACTGACT | |
| | TACAAAAGTACTGTCTTAATTGGAG | |
| | ATATCAAAGCGTGAAGCTCTTTGCA | |
| | CGCCAATTGAATCGTCTATTCGGGT | |
| | TACAGCATGGTTTTGAATGGATCCA | |
| | TTGTATCCTCATGCAGTCCACCATG | |
| | TATGTAGCTGATCCCTTCAATCCTC | |
| | CAAACGGGAACGCAAGCCCAAATTT | |
| | AGATGATAACCCAAATAATGACATC | |
| | TTTATTGTATCACCTCGAGGAGCAA | |
| | TTGAGGGCCTGTGTCAGAAGATGTG | |
| | GACAATTATATCAATCTCAGCAATT | |
| | CATGCAGCTGCAGCTGTAGCAGGCC | |
| | TAAGAGTCGCATCAATGGTTCAAGG | |
| | TGACAACCAGGTTATCGGTGTCACT | |
| | CGAGAATTCCTTGCAGGACATGATC | |
| | AAAGTCATGTGGATAGTCAACTTAC | |
| | TGCATCATTAGAAAACTTTACACAA | |
| | ATATTCAAGGAGATAAATTATGGGC | |
| | TTGGCCATAACCTCAAATTACGGGA | |
| | AACAATTAAGTCTAGTCACATGTTC | |
| | ATTTATTCTAAAAGAATTTTTTACG | |
| | ATGGGAGGATTCTCCCTCAATTGTT | |
| | AAAGAATATAAGTAAACTAACTTTG | |
| | TCGGCAACTACAACAGGGGAGAATT | |
| | GCTTAACTAGCTGTGGGGACTTATC | |
| | TTCATGTATTACCCGCTGTATTGAG | |
| | AATGGTTTCCCAAAGGATGCTGCAT | |
| | TCATTCTAAATCAGCTTACAATTAG | |
| | GACTCAGATACTTGCAGACCATTTT | |
| | TACTCAATACTTGGTGGGTGCTTCA | |
| | CTGGGCTAAATCAACATGATATTCG | |
| | CTTACTGCTCTCTGATGGTTCTATA | |
| | TTGCCAGCTCAGCTGGGGGGATTTA | |
| | ACAACTTGAATATATCCCGATTATT | |
| | CTGTAGAAATATAGGTGACCCTCTA | |
| | GTAGCCTCAATTGCAGATACAAAAC | |
| | GCTATGTGAAATGCGGCCTTTTGAC | |
| | TCCATCTATACTTGACTCAGTCGTC | |
| | TCCATCACTGATAGGAAAGGCTCAT | |
| | TTACTACCCTGATGATGGATCCCTA | |
| | TTCAATCAATCTCGATTATATTCAA | |
| | CAGCCAGAAACCCGCTTAAAACGTC | |
| | ATGTGCAGAAAGTTCTCCTTCAAGA | |
| | ATCAGTAAATCCTCTACTGCAGGGC | |
| | GTATTTCTCGAGACTCAGCAGGATG | |
| | AAGAGGAAGCACTAGCTGCGTTTTT | |
| | ATTAGACAGAGATATTGTGATGCCC | |
| | CGTGTAGCTCACGCAATTTTTGAAT | |
| | GTACGAGTCTCGGACGCCGTAGACA | |
| | CATACAGGGGCTGATTGATACAACA | |
| | AAGACTATAATAGCCCTGGCATTGG | |
| | ACACACAGAATCTGAGTCACACTAA | |
| | GCGTGAGCAAATAGTTACGTATAAT | |
| | GCAACCTATATGAGGTCCTTAACAC | |
| | AAATGCTTAAATTAAGCAGAACTGT | |
| | TCATAAGGGGATGACCAGGATGCTG | |
| | CCTATTTTCAATATCAATGATTGTT | |
| | CTGTAATACTAGCACAACAAGTTAG | |
| | GCGTGCAAGCTGGGCTCCGCTGCTA | |
| | AATTGGCGCACCTTGGAAGGGCTTG | |
| | AGGTCCCTGATCCAATTGAATCCGT | |
| | GTCTGGATACCTTGGTCTTGACTCC | |
| | AACAATTGCTTCCTCTGTTGCCATG | |
| | AACAAAATAGCTACTCTTGGTTTTT | |
| | CCTCCCCAAATTGTGCCATTTTGAC | |
| | GATTCGAGACAATCATACTCAACCC | |
| | AACGTGTACCTTATATAGGTTCAAA | |
| | AACAGATGAGAGACAAATGTCTACA | |
| | ATTAACCTCCTAGAGAAAACAACCT | |
| | GTCATGCCCGTGCCGCAACAAGGTT | |
| | AGCGTCATTATATATATGGGCATAT | |
| | GGTGATTCGGAAGACAGCTGGGATG | |
| | CAGTAGAATCACTATCAAATAGCCG | |
| | ATGCCAAATTACACGAGAGCAATTG | |
| | CAGGCCCTTTGCCCCATGCCGTCAT | |
| | CAGTAAATTTACATCATAGACTCAA | |
| | TGACGGTATTACCCAAGTTAAGTTC | |
| | ATGCCATCAACAAACAGCAGAGTAT | |
| | CCAGATTTGTACATATTTCTAATGA | |
| | CAGGCAGAATTACGTCCTGGACGAC | |
| | ACTGTCACTGATAGTAACTTGATAT | |
| | ATCAGCAGGTCATGCTTTTGGGTTT | |
| | GAGCATATTGGAGACATACTTTCGA | |
| | GAACCAACAACTGTGAACTTGTCGA | |
| | GTATCGTCCTCCATTTGCATACTGA | |
| | CGTGTCCTGTTGTCTCCGTGAATGC | |
| | CCTATGACACAGTATGCACCACCAC | |
| | TCAGAGACCTCCCTGAACTAACCAT | |
| | AACAATGACAAATCCATTCCTTTAT | |
| | GACCAAGCACCTATCAGTGAAGCAG | |
| | ATCTATGTCGGCTTTCGAAGGTAGC | |
| | CTTCCGTAAAGCAGGAGACAATTAT | |
| | GAACTATATGATCAATTCCAACTGC | |
| | GATCCACACTCTCTTCAACCACAGG | |
| | GAAGGATGTTGCGGCAACTATTTTT | |
| | GGACCACTTGCGGCAGTATCTGCAA | |
| | AAAATGATGCAATTGTTACTAATGA | |
| | CTACAGTGGTAACTGGATCTCAGAG | |
| | TTCAGGTACAGTGATTACTACCTAC | |
| | TGAGTACGAGTTTGGGTTACGAGAT | |
| | TTTACTAATATTTGCTTACCAACTC | |
| | TACTATCTAAGGATTAGGTATAAGC | |
| | AAAACATCATTTGTTACATGGAGTC | |
| | TGTATTCCGCCGTTGCCACTCATTA | |
| | TGCTTAGGTGACCTGATTCAAACAA | |
| | TCTCCCACTCAGAAATACTGACTGG | |
| | ATTAAATGCTGCAGGCTTCAACTTG | |
| | ATGTTGGATAGGAGTGATTTGAAGA | |
| | ATAACCAATTGTCTCGCCTAGCCGT | |
| | CAAGTATCTCACGCTCTGTGTCCAG | |
| | GCTGCCATTAACAACTTGGAGGTTG | |
| | GCTCAGAACCTCTCTGTATTATTGG | |
| | AGGTCAACTCGATGATGACATCTCG | |
| | TTTCAGGTAGCGCATTTTCTATGTA | |
| | GAAGGCTTTGCATTCTAAGTCTTGT | |
| | ACACTCAAATTTACAGAATCTCCCC | |
| | ACGATCCGTGATAATGAGGTTGATG | |
| | TGAAATCTAAATTAATTTATGACCA | |
| | TCTCAAACTGGTTGCTACAACTTTG | |
| | AATGATCGAGACCAATCGTATCTGT | |
| | TAAAGCTGTTAAATAACCCAAATTT | |
| | GGAATTACACACACCGCAAGTCTAC | |
| | TTCATAATGAGGAAGTGTCTAGGTT | |
| | TGCTCAAGGCGTATGGCGCAGTACC | |
| | ATACAAACAACCTTTTCCAACATCA | |
| | CCTATTGTACCATTCCCTAATCTGA | |
| | GTGGGTCTAAGTGGCACCTTGAACG | |
| | TGTTATAGACAGTATTGAGGCACCA | |
| | AAATCTTACACTTGGGTTCCTAACA | |
| | CAACACTCCCACTGGCCAAGGATCA | |
| | TGTATCCCCCAATCCAAGCAGAATT | |
| | CTTGACAAAATCAACTTGTTTAGAT | |
| | CACTGAGCCCCAGACACTCAGTTTG | |
| | GTACCGTAATCGTCAATACAAACTT | |
| | ATCCTTTCCCAGCTGAGTCATGATA | |
| | TTCTTGGGGGCTCTACACTTTACCT | |
| | AGGTGAAGGAGGGGGCTCAACTATC | |
| | CTCACAATTGAACCCCACATTAGAA | |
| | GTGACAAAATATACTACCATACATA | |
| | CTTCCCTGCCGATCAGAGTCCGGCT | |
| | CAACGCAACTTTATACCCCAGCCTA | |
| | CGACATTCTTGAGATCTAACTTTTA | |
| | TCACTTTGAACTGGAACCATCAGGA | |
| | TGTGAGTTTGTAAATTGCTGGTCTG | |
| | AGGATGCAAACGCCACAAATCTTAC | |
| | AGAACTTAGGTGTATTAACCACATC | |
| | ATGACAGTGATACCAGTTGGCTCGT | |
| | TAAACAGAATCATATGTGACATAGA | |
| | GCTAGCTAGAGACACATCAATCAAG | |
| | TCGATAGCCMCMGTTTATCTTAATC | |
| | TAGGAATTCTAGCTCATGCATTGCT | |
| | TAGTCCAGGGGGAATCTGCATATGC | |
| | AGGTGCCATTTACTGAACGCTTCAA | |
| | ATCTTGCGATTGTATCTTTTGTACT | |
| | AAAAACATTGTCAAGCAAGCTGGCA | |
| | ATTTCATTCTCTGGATTTAGCGGTG | |
| | TGAATGATCCTTCTTGTGTGGTTGG | |
| | AACTACCAAGGAAAGCACTATTAGC | |
| | TTAGATGTTCTCAGTTCAATTGCTT | |
| | CTGCATTCATAAACGAATTGACATC | |
| | GAATGAAGTACCGATTCCCCAAGAG | |
| | GTATTGACATTACTATCTTGTTACA | |
| | CAGAGCAGCTAGGGAACTTAGGGCA | |
| | ATTGATTGAGAAAACCTGGATCCGC | |
| | GAGATACGGAAACCGCATTTAATGC | |
| | AGTGTGAAATGGAGTGGATCGGGCT | |
| | TTTGGGAAATGATGCATTGAGTGAC | |
| | GTAGACAATTTCCTGAACTATTACA | |
| | ACCCATCATGCTCATCAGTTCCAGA | |
| | ACTAATTACACCTACAGTTAGTTCA | |
| | TTGCTTTTTGAACTGGTTAGCCTAA | |
| | CTCCAGAAGTCTGCTCTTACGATGA | |
| | ATCTAATTATAAACGAACAATTCAG | |
| | GTAGGGCAGGCATATAACATTACAG | |
| | TTTCTGGCAAAGTAAGCACTATGAT | |
| | AAGGACCTGTTGCGAACAATGCATT | |
| | AAGCTTCTAATAGCTAATAGTGAAG | |
| | TACTAATTGATACTGATTTGGCGTA | |
| | TCTTGTTAGAGGCATTCGCGATGGG | |
| | TCATTCACTCTAGGCTCGATCATAA | |
| | GCCAAAACCAAATACTAAAAGCATC | |
| | CAGAGCACCACGTTACCTCAAAACA | |
| | CCCAAAATTCAATTATGGGTATCAA | |
| | CACTGTTAGCCATTAGGATTGAGGA | |
| | AGTCTTCTCACGCCATTATAGAAAG | |
| | GTCCTCTTACGATCAATCCGCCTTT | |
| | TGTCACTCTACAAGTATCTCCAGGA | |
| | CAAGACGAAGTAGATAACCATTTAT | |
| | CATAGAGTCAGACGGGTTCTAGTTC | |
| | AATCCCTGCGTTATTCTTCGCTCAC | |
| | AGAATCTTGGATTCCATCCGGGGCT | |
| | GTGCTGACATAATATGTAAATATGT | |
| | AATATATTGGTTACTGGACATAATC | |
| | AATGAGGCTTCTGTAGTATTTATCC | |
| | CAACTCCTTAATATTAGTTTCAAAA | |
| | TGAGAACATTATATGTTAATAAAAA | |
| | ACTAAAAATGATAACCAGTTGAATC | |
| | TGGACCGAACTGGCAATTGCATAAA | |
| | AAATAAAAAATTTATTAAAATTAAA | |
| | ATTGAAATCATATAACAACACGTTT | |
| | AAGGGGAATAAAAACAAGATTGGGA | |
| | ATAAAAATAATAATAATAAAAGGAA | |
| | TAAAACAAAAAATAAAAATAAAAAT | |
| | GGGAATAAAAATAAAAATAAAAATA | |
| | AAGAAAAAAATGGGAGAAAAGCTCC | |
| | AATTAACAAACAAATCAAAACTAAA | |
| | CTTAAGATTACAACTAAAAATACAA | |
| | ATATTAACAAAAATAGACTGAGAAG | |
| | TAGAATCGTAAATAAGACCGGCAGT | |
| | CAGTTTAGTATGGAAAATAAGACCC | |
| | AGATTACTTACACATCCTGCCTTAG | |
| | TTTCCCCCTTATTTAATTTTAAGTG | |
| | GATTTAGGGAGTCACTGATCCAGCT | |
| | AAGAACCTATTTTCTTATAGCTAAA | |
| | ATCTCAATCTTGATGTCTCCAATCA | |
| | ATTAAAACCGGTTGTTTAATTAAGT | |
| | TGTTCCTAATCAATTCACCTCAGTA | |
| | GATCCAGTGTGAATCGCACTGGTCC | |
| | AATCCAACATGGGTCTAATTAAATA | |
| | AAACGACTGTAATAGGTCGAATGCG | |
| | GCCTCGATCAACAGAGTAACAAACA | |
| | TTACAAATTACAAATCAGAGTTGTT | |
| | AATTAAACCATTTATATAACTTTTT | |
| | GTTTAGT | |
| Avian | GCGAAAAAGAAGAATAAAAGGCAGA | SEQ ID |
| paramyxovir | AGCCTTTTAAAAGGAACCCTGGGCT | No: 3 |
| us 4 strain | GTCGTAGGTGTGGGAAGGTTGTATT | |
| APMV4/ | CCGAGTGCGCCTCCGAGGCATCTAC | |
| mallard/ | TCTACACCTATCACAATGGCTGGTG | |
| Belgium/ | TCTTCTCCCAGTATGAGAGGTTTGT | |
| 15129/07 | GGACAATCAATCCCAAGTATCAAGG | |
| complete | AAGGATCATCGGTCCCTGGCAGGGG | |
| genome | GATGCCTTAAAGTCAACATCCCTAT | |
| Genbank: | GCTTGTCACTGCATCTGAAGATCCC | |
| JN571485.1 | ACCACTCGTTGGCAACTAGCATGTT | |
| | TATCTCTAAGGCTCTTGATCTCCAA | |
| | CTCATCAACCAGTGCTATCCGACAG | |
| | GGGGCAATACTGACTCTCATGTCAC | |
| | TACCGTCACAAAATATGAGAGCAAC | |
| | GGCAGCTATTGCTGGTTCCACAAAT | |
| | GCAGCTGTTATCAACACTATGGAAG | |
| | TCTTGAGTGTCAATGACTGGACCCC | |
| | ATCCTTCGACCCTAGGAGCGGTCTC | |
| | TCTGAAGAGGATGCTCAGGTTTTCA | |
| | GAGACATGGCAAGGGACCTGCCCCC | |
| | TCAGTTCACCTCCGGATCACCCTTT | |
| | ACATCAGCATTGGCGGAGGGGTTTA | |
| | CCCCAGAAGACACCCACGACCTAAT | |
| | GGAGGCCTTGACCAGTGTGCTGATA | |
| | CAGATCTGGATCCTGGTGGCTAAGG | |
| | CCATGACCAACATTGATGGCTCTGG | |
| | GGAGGCCAATGAGAGACGTCTTGCA | |
| | AAGTACATCCAAAAGGGACAGCTTA | |
| | ATCGCCAGTTTGCAATTGGTAATCC | |
| | TGCTCGTCTGATAATCCAACAGACG | |
| | ATCAAAAGCTCCTTAACTGTCCGCA | |
| | GGTTCTTGGTCTCTGAGCTTCGTGC | |
| | ATCACGAGGTGCAGTGAAAGAAGGA | |
| | TCCCCTTACTATGCAGCTGTTGGGG | |
| | ATATCCACGCTTACATCTTTAACGC | |
| | AGGACTGACACCATTCTTGACTACC | |
| | TTAAGATATGGGATAGGCACCAAGT | |
| | ATGCTGCTGTTGCACTCAGTGTGTT | |
| | CGCTGCAGACATTGCAAAATTAAAG | |
| | AGCCTACTTACCCTGTACCAAGACA | |
| | AGGGTGTGGAGGCCGGATACATGGC | |
| | ACTCCTTGAAGATCCAGATTCCATG | |
| | CACTTTGCACCCGGAAATTTCCCAC | |
| | ACATGTACTCCTATGCGATGGGGGT | |
| | GGCTTCTTACCATGACCCCAGCATG | |
| | CGCCAATACCAATATGCCAGGAGGT | |
| | TCCTCAGCCGTCCCTTCTACTTGCT | |
| | AGGGAGGGACATGGCCGCCAAGAAC | |
| | ACAGGCACGCTGGATGAGCAACTGG | |
| | CAAAGGAACTGCAAGTGTCAGAAAG | |
| | AGACCGCGCCGCATTGTCCGCTGCG | |
| | ATTCAATCAGCAATGGAGGGGGGAG | |
| | AATCTGACGACTTCCCACTGTCGGG | |
| | ATCCATGCCGGCTCTCTCCGACACT | |
| | GCGCAACCAGTTACCCCAAGAACCC | |
| | AACAGTCCCAGCTTTCCCCTCCACA | |
| | ATCATCAAGCATGTCTCAATCAGCG | |
| | CCCAGGACCCCGGACTACCAGCCTG | |
| | ATTTTGAACTGTAGGCTGCATCCAC | |
| | GCACCAACAACAGGCAAAAGAAATC | |
| | ACCCTCCTCCCCACACATCCCACCC | |
| | ACTCACCCGCCGAGATCCAATCCAA | |
| | CACCCCAGCATCCCCATCATTTAAT | |
| | TAAAAACTGACCAATAGGGTGGGGA | |
| | AGGAGAGTTATTGGCTGTTGCCAAG | |
| | TTTGTGCAGCAATGGATTTCACCGA | |
| | CATTGATGCTGTCAACTCATTAATT | |
| | GAATCATCATCAGCAATCATAGATT | |
| | CCATACAGCATGGAGGGCTGCAACC | |
| | ATCGGGCACTGTCGGCCTATCGCAA | |
| | ATCCCAAAGGGGATAACCAGCGCTT | |
| | TAACTAAGGCCTGGGAGGCTGAGGC | |
| | AGCAACTGCTGGCAATGGGGACACC | |
| | CAACACAAACCTGACAGTCCGGAGG | |
| | ATCATCAGGCCAACGACACAGACTC | |
| | CCCCGAAGACACAGGCACCAACCAG | |
| | ACCATCCAGGAAGCCAATATCGTTG | |
| | AAACACCCCACCCCGAAGTGCTATC | |
| | GGCAGCCAAAGCCAGACTCAAGAGG | |
| | CCCAAGGCAGGGAGGGACACCCACG | |
| | ACAATCCCTCTGCGCAACCTGATCA | |
| | TTTTTTAAAGGGGGGCCCCCTGAGC | |
| | CCACAACCAGCGGCACCATGGGTGC | |
| | AAAGTCCACCCATTCATGGAGGTCC | |
| | CGGCACCGTCGATCCCCGCCCATCA | |
| | CAAACTCAGGATCATTCCCTCACCG | |
| | GAGAGAAATGGCAATCGTCACCGAC | |
| | AAAGCAACCGGAGACATTGAACTGG | |
| | TGGAATGGTGCAACCCGGGGTGCAC | |
| | CGCAATCCGAACTGAACCAACCAGA | |
| | CTCGACTGTGTATGCGGACACTGCC | |
| | CCACCATCTGCAGCCTCTGCATGTA | |
| | TGACGACTGATCAGGTACAACTATT | |
| | AATGAAGGAGGTTGCCGATATGAAA | |
| | TCACTCCTTCAGGCATTAGTAAAGA | |
| | ACCTAGCTGTCCTGCCTCAACTAAG | |
| | GAACGAGGTTGCAGCAATCAGGACA | |
| | TCACAGGCCATGATAGAGGGGACAC | |
| | TCAATTCAATCAAGATTCTCGATCC | |
| | TGGGAATTATCAAGAATCATCACTA | |
| | AACAGCTGGTTCAAACCACGCCAAG | |
| | ATCACGCGGTTGTTGTGTCCGGACC | |
| | AGGGAATCCATTGACCATGCCAACC | |
| | CCAATCCAAGACAACACCATATTCC | |
| | TGGATGAACTGGCAAGACCTCATCC | |
| | TAGTTTGGTCAATCCGTCCCCGCCC | |
| | ACTACCAACACTAATGTTGATCTTG | |
| | GCCCACAGAAGCAGGCTGCGATAGC | |
| | TTATATCTCAGCAAAATGCAAGGAT | |
| | CCAGGGAAACGAGATCAGCTCTCAA | |
| | AGCTCATCGAGCGAGCAACCACCTT | |
| | GAGCGAGATCAACAAAGTCAAAAGA | |
| | CAGGCCCTCGGCCTCTAGATCACTC | |
| | GACCACCCCCAGTAATGAATACAAC | |
| | AATAATCAGAACCCCCCTAAAACAC | |
| | ATGGTCAACCCAACACACCACCCGC | |
| | ACCACCCGCTACTATCCTTTGCCAG | |
| | AAACTCCGCCGCAGCCGATTTATTC | |
| | AAAAGAAGCCATTTGATATGACTTA | |
| | GCAACCGCAAGATAGGGTGGGGAAG | |
| | GTGCTTTGCCTGCAAGAGGGCTCCC | |
| | TCATCTTCAGACACGTACCCGCCAA | |
| | CCCACCAGTGACGCAATGGCAGACA | |
| | TGGACACCGTATATATCAATCTGAT | |
| | GGCAGATGATCCAACCCACCAAAAA | |
| | GAACTGCTGTCCTTTCCCCTCGTTC | |
| | CCGTGACTGGTCCTGACGGGAAAAA | |
| | GGAACTCCAACACCAGGTCCGGACT | |
| | CAATCCTTGCTCGCCTCAGACAAGC | |
| | AAACTGAGAGGTTCATCTTCCTCAA | |
| | CACTTACGGGTTTATCTATGACACT | |
| | ACACCGGACAAGACAACTTTTTCCA | |
| | CCCCAGAGCACATCAATCAGCCCAA | |
| | GAGAACGATGGTGAGTGCTGCGATG | |
| | ATGACCATTGGCCTGGTCCCCGCCA | |
| | ATATACCCTTGAACGAATTAACAGC | |
| | TACTGTGTTCGGCCTGAAAGTAAGA | |
| | GTGAGGAAGAGTGCGAGATATCGAG | |
| | AGGTGGTCTGGTATCAGTGCAATCC | |
| | TGTACCAGCCCTGCTTGCAGCCACC | |
| | AGGTTCGGTCGCCAAGGAGGTCTCG | |
| | AATCAAGCACTGGAGTCAGCGTAAA | |
| | GGCCCCCGAGAAGATAGATTGCGAG | |
| | AAGGATTATACTTACTACCCTTATT | |
| | TCCTATCTGTGTGCTACATCGCCAC | |
| | TTCCAACCTGTTCAAGGTACCAAAA | |
| | ATGGTTGCTAATGCGACCAACAGTC | |
| | AATTATACCACCTGACTATGCAGGT | |
| | CACATTTGCCTTTCCAAAAAACATC | |
| | CCCCCAGCTAACCAGAAACTTCTGA | |
| | CACAAGTGGATGAAGGATTCGAGGG | |
| | CACTGTGGACTGCCATTTTGGGAAC | |
| | ATGCTGAAAAAGGATCGGAAAGGGA | |
| | ATATGAGGACATTGTCGCAGGCGGC | |
| | AGACAAGGTCAGACGGATGAATATC | |
| | CTTGTTGGTATCTTTGACTTGCATG | |
| | GGCCGACACTCTTCCTGGAGTATAC | |
| | TGGGAAACTAACAAAAGCTCTGTTA | |
| | GGGTTCATGTCTACTAGCCGAACAG | |
| | CAATCATCCCCATATCTCAGCTCAA | |
| | TCCTATGCTGGGTCAACTTATGTGG | |
| | AGCAGTGATGCCCAGATAGTAAAAT | |
| | TAAGAGTGGTCATAACTACATCCAA | |
| | ACGCGGCCCATGCGGGGGTGAGCAG | |
| | GAGTATGTGCTGGATCCCAAATTCA | |
| | CAGTTAAAAAAGAGAAAGCCCGACT | |
| | CAACCCTTTCAAGAAGGCAGCCCAA | |
| | TGATCAAATCTGCAGGATCTCAAGA | |
| | ATCAGACCACTCTATACTATTCACC | |
| | GATCAATAGACATGTAACTATACAG | |
| | TTGATGGACCTATGAAGAATCAATT | |
| | AGCAAACCGAATCCTTACTAGGGTG | |
| | GGGAAGGAGTTGATTGGGTGTCTAA | |
| | ACAAAAGCATTCCTTTACACCTCCT | |
| | CGCTACGAAACAACCATAATGAGGT | |
| | TATCACGCACAATCCTGACTTTGAT | |
| | TCTCAGCACACTTACCGGCTATTTA | |
| | ATGAATGCCCACTCCACCAATGTGA | |
| | ATGAGAAACCAAAGTCTGAGGGGAT | |
| | TAGGGGGGATCTTATACCAGGCGCA | |
| | GGTATTTTTGTAACTCAAGTCCGAC | |
| | AACTACAGATCTACCAACAGTCTGG | |
| | GTATCATGACCTTGTCATCAGGTTA | |
| | TTACCTCTTCTACCGGCAGAACTTA | |
| | ATGATTGTCAAAGGGAAGTTGTCAC | |
| | AGAGTACAACAACACGGTATCACAG | |
| | CTGTTGCAGCCTATCAAAACCAACC | |
| | TGGATACCTTATTGGCTGATGGTAG | |
| | CACAAGGGATGCCGATATACAGCCA | |
| | CGGTTCATTGGGGCAATAATAGCCA | |
| | CAGGTGCCCTGGCGGTGGCTACGGT | |
| | AGCTGAGGTGACTGCAGCCCAAGCA | |
| | CTATCTCAGTCGAAAACAAACGCTC | |
| | AAAATATTCTCAAGTTGAGAGATAG | |
| | TATTCAGGCTACCAACCAAGCAGTT | |
| | TTCGAAATTTCACAAGGACTCGAGG | |
| | CAACTGCAACTGTGCTATCAAAACT | |
| | GCAAACTGAGCTCAATGAGAACATT | |
| | ATCCCAAGCCTGAACAACTTGTCCT | |
| | GTGCTGCCATGGGGAATCGCCTTGG | |
| | TGTATCACTATCACTCTACTTGACC | |
| | TTAATGACCACTCTATTTGGGGACC | |
| | AGATCACAAACCCAGTGCTGACACC | |
| | AATCTCCTATAGCACTCTATCGGCA | |
| | ATGGCAGGCGGTCACATTGGCCCGG | |
| | TGATGAGTAAAATATTAGCTGGATC | |
| | TGTCACAAGTCAGTTGGGGGCAGAA | |
| | CAGTTGATTGCTAGCGGCTTAATAC | |
| | AGTCACAGGTAGTAGGTTATGATTC | |
| | CCAATATCAATTATTGGTTATCAGG | |
| | GTCAACCTTGTACGGATTCAAGAGG | |
| | TCCAGAATACGAGGGTCGTATCACT | |
| | AAGAACACTAGCGGTCAATAGGGAT | |
| | GGTGGACTTTATAGAGCCCAGGTGC | |
| | CTCCCGAGGTAGTTGAACGGTCTGG | |
| | CATTGCAGAGCGATTTTATGCAGAT | |
| | GATTGTGTTCTTACTACAACTGATT | |
| | ACATTTGCTCATCGATCCGATCTTC | |
| | TCGGCTTAATCCAGAGTTAGTCAAG | |
| | TGTCTCAGTGGTGCACTTGATTCAT | |
| | GCACATTTGAGAGGGAAAGTGCATT | |
| | ATTGTCGACCCCTTTCTTTGTATAC | |
| | AACAAGGCAGTCGTCGCAAATTGTA | |
| | AAGCAGCAACATGTAGATGTAATAA | |
| | ACCGCCATCTATTATTGCCCAATAC | |
| | TCTGCATCAGCTCTAGTCACCATCA | |
| | CCACCGACACCTGTGCCGACCTTGA | |
| | AATTGAGGGTTATCGCTTCAACATA | |
| | CAGACTGAATCCAACTCATGGGTTG | |
| | CACCAAACTTCACGGTCTCGACTTC | |
| | ACAGATTGTATCAGTTGATCCAATA | |
| | GACATCTCCTCTGACATTGCCAAAA | |
| | TCAACAGTTCCATCGAGGCTGCGAG | |
| | AGAGCAGCTGGAACTGAGCAACCAG | |
| | ATCCTTTCCCGGATCAACCCACGAA | |
| | TTGTGAATGATGAATCACTGATAGC | |
| | TATTATCGTGACAATTGTTGTGCTT | |
| | AGTCTCCTTGTAATCGGTCTGATTG | |
| | TTGTTCTCGGTGTGATGTATAAGAA | |
| | TCTTAAGAAAGTCCAACGAGCTCAA | |
| | GCTGCCATGATGATGCAGCAAATGA | |
| | GCTCATCACAGCCTGTGACCACTAA | |
| | ATTAGGGACGCCTTTCTAGGAGAAT | |
| | AATCATATCACTCTACTCAATGATG | |
| | AGCAAAACGTACCAATCGTCAATGA | |
| | TTGTGTCACGAGGCCGGTTGGGAAT | |
| | GCATCGAATCTCTCCCCTTTCTTTT | |
| | TAATTAAAAACATTTGAAGTGAGGG | |
| | TGAGAGGGGGGGAGTGTATGGTAGG | |
| | GTGGGGAAGGTAGCCAATTCCTGCC | |
| | TATTGGGCCGACCGTATCAAAAGAA | |
| | CTCAACAGAAGTCTAGATACAGGGT | |
| | GACATGGAGGGCAGCCGTGATAATC | |
| | TTACAGTGGATGATGAATTAAAGAC | |
| | AACATGGAGGTTAGCTTATAGAGTT | |
| | GTGTCCCTTCTATTGATGGTGAGCG | |
| | CTTTGATAATCTCTATAGTAATCCT | |
| | GACAAGAGATAACAGCCAAAGCATA | |
| | ATCACAGCGATCAACCAGTCATCCG | |
| | ACGCAGACTCAAAGTGGCAAACGGG | |
| | AATAGAAGGGAAAATCACCTCCATT | |
| | ATGACTGATACGCTCGATACCAGGA | |
| | ATGCAGCCCTTCTCCACATTCCACT | |
| | CCAGCTCAACACGCTTGAGGCGAAC | |
| | CTTTTGTCCGCCCTTGGGGGCAACA | |
| | CAGGAATTGGTCCCGGGGATCTAGA | |
| | TCACTGCCGTTACCCTGTTCATGAC | |
| | TCCGCTTACCTGCATGGAGTTAATC | |
| | GATTACTCATCAACCAGACAGCTGA | |
| | TTACACAGCAGAAGGCCCCCTAGAT | |
| | CATGTGAACTTTATTCCAGCCCCGG | |
| | TTACGACCACTGGATGCACAAGGAT | |
| | ACCATCCTTTTCCGTGTCATCGTCC | |
| | ATTTGGTGCTATACACACAACGTGA | |
| | TCGAAACCGGTTGCAATGACCACTC | |
| | AGGTAGTAACCAATATATCAGCATG | |
| | GGAGTCATTAAGAGAGCGGGCAACG | |
| | GCCTACCTTACTTCTCGACAGTTGT | |
| | AAGTAAATATCTGACTGATGGGTTG | |
| | AATAGGAAAAGCTGTTCTGTAGCCG | |
| | CCGGATCCGGGCATTGCTACCTCCT | |
| | TTGCAGCTTAGTGTCGGAACCCGAA | |
| | CCTGATGACTATGTGTCACCTGATC | |
| | CCACACCGATGAGGTTAGGGGTGCT | |
| | AACGTGGGATGGGTCTTACACTGAA | |
| | CAGGTGGTACCCGAAAGAATATTCA | |
| | AGAACATATGGAGTGCAAACTACCC | |
| | AGGAGTAGGGTCAGGTGCTATAGTA | |
| | GGGAATAAGGTGTTATTCCCATTTT | |
| | ACGGCGGAGTGAGAAATGGATCGAC | |
| | CCCGGAGGTGATGAATAGGGGAAGA | |
| | TACTACTACATCCAGGATCCAAATG | |
| | ACTATTGTCCTGACCCGCTACAAGA | |
| | TCAGATCTTAAGGGCGGAACAATCG | |
| | TATTACCCAACTCGATTTGGTAGGA | |
| | GGATGGTAATGCAAGGGGTCCTAGC | |
| | ATGTCCAGTATCCAACAATTCAACA | |
| | ATAGCAAGCCAATGTCAATCTTACT | |
| | ATTTTAATAACTCATTAGGATTCAT | |
| | TGGGGCAGAATCTAGAATCTATTAC | |
| | CTCAATGGTAACATTTACCTTTATC | |
| | AGAGAAGCTCGAGCTGGTGGCCTCA | |
| | TCCCCAGATTTACCTGCTTGATTCC | |
| | AGGATTGCAAGTCCGGGTACTCAGA | |
| | ACATTGACTCAGGTGTTAATCTCAA | |
| | GATGTTAAATGTTACTGTGATTACA | |
| | CGACCATCATCTGGTTTTTGTAATA | |
| | GTCAGTCACGATGCCCTAATGACTG | |
| | CTTATTCGGGGTCTACTCGGATATC | |
| | TGGCCTCTTAGCCTTACCTCAGATA | |
| | GCATATTCGCGTTCACAATGTATTT | |
| | ACAGGGGAAGACAACACGTATTGAC | |
| | CCGGCTTGGGCACTATTCTCCAATC | |
| | ATGCGATTGGGCATGAGGCTCGTCT | |
| | GTTCAATAAGRAGGTTAGTGCTGCT | |
| | TATTCTACCACCACTTGTTTTTCGG | |
| | ACACTATCCAAAATCAGGTGTATTG | |
| | CCTGAGTATACTTGAGGTCAGGAGT | |
| | GAGCTCTTGGGAGCATTCAAAATAG | |
| | TACCATTCCTCTATCGCGTCTTGTA | |
| | GGCATCCATTCAGCCAAAAAACTTG | |
| | AGTGACCATGAGGTTAACACCTGAT | |
| | CCCCTTCAAAAACATCTATCTTAAT | |
| | TACCGTTCTAGATCCATGATTAGGT | |
| | ACCTTTCCAATCAATCATTTGGTTT | |
| | TTAATTAAAAACGAAAGAATGGGCC | |
| | TAGTTCCAAGAAAGGGCTGGAACCC | |
| | ATTAGGGTGGGGAAGGATTGCTTTG | |
| | CTCCTTGACTCACACCTGCGTACAC | |
| | TCGATCTCACTTCTATAAAGAAGGA | |
| | ATCCTTCTCAAATTCGCCCCACAAT | |
| | GTCCAATCAGGCAGCTGAGATTATA | |
| | CTACCCACCTTCCATCTAGAATCAC | |
| | CCTTAATCGAGAATAAGTGCTTCTA | |
| | TTATATGCAATTACTTGGTCTCGTG | |
| | TTGCCACATGATCACTGGAGATGGA | |
| | GGGCATTCGTTAACTTTACAGTGGA | |
| | TCAGGTGCACCTTAAAAATCGTAAT | |
| | CCCCGCTTAATGGCCCACATCGACC | |
| | ACACTAAAGATAGATTAAGGACTCA | |
| | TGGTGTCTTAGGTTTCCACCAGACT | |
| | CAGACAAGTATGAGCCGTTACCGTG | |
| | TTTTGCTTCATCCTGAAACCTTACC | |
| | TTGGCTATCAGCCATGGGAGGATGC | |
| | ATCAATCAGGTTCCTAAAGCATGGC | |
| | GGAACACTCTGAAATCGATCGAGCA | |
| | CAGTGTAAAGCAGGAGGCACCTCAA | |
| | CTAAAGTTACTCATGGAGAGAACCT | |
| | CATTAAAATTAACTGGAGTACCTTA | |
| | CTTGTTCTCTAATTGCAATCCCGGG | |
| | AAAACCACAGCAGGAACTATGCCTG | |
| | TCCTAAGTGAGATGGCATCGGAACT | |
| | CTTATCAAATCCTATCTCCCAATTC | |
| | CAATCAACATGGGGGTGTGCTGCTT | |
| | CGGGGTGGCACCATGTAGTCAGTAT | |
| | CATGAGGCTCCAACAATATCAAAGA | |
| | AGGACAGGTAAGGAAGAGAAAGCAA | |
| | TCACTGAAGTTCAGTATGGCACGGA | |
| | CACCTGTCTCATTAACGCAGACTAC | |
| | ACCGTTGTTTTTTCCACACAGAACC | |
| | GTGTTATAACGGTCTTGCCTTTCGA | |
| | TGTTGTCCTCATGATGCAAGACCTG | |
| | CTAGAATCCCGACGGAATGTCCTGT | |
| | TCTGTGCCCGCTTTATGTATCCCAG | |
| | AAGCCAACTTCATGAGAGGATAAGT | |
| | ACAATATTAGCCCTTGGAGACCAAC | |
| | TGGGGAGAAAAGCACCCCAAGTCCT | |
| | GTATGATTTTGTAGCAACCCTTGAG | |
| | TCATTTGCATACGCAGCTGTTCAAC | |
| | TTCATGACAACAATCCTACCTACGG | |
| | TGGGGCCTTCTTTGAATTCAATATC | |
| | CAAGAGTTAGAATCTATTCTGTCCC | |
| | CTGCACTTAGTAAGGATCAGGTCAA | |
| | CTTCTACATAGGTCAAGTTTGCTCA | |
| | GCGTACAGTAACCTTCCTCCATCTG | |
| | AATCGGCAGAATTGCTGTGCCTGCT | |
| | ACGCCTGTGGGGTCATCCCTTGCTA | |
| | AACAGCCTTGATGCAGCAAAGAAAG | |
| | TCAGGGAATCTATGTGTGCCGGGAA | |
| | GGTTCTCGATTACAACGCCATTCGA | |
| | CTCGTCTTGTCTTTTTATCATACGT | |
| | TACTAATCAATGGGTATCGGAAGAA | |
| | GCACAAGGGTCGCTGGCCAAATGTG | |
| | AATCAACATTCACTCCTCAACCCGA | |
| | TAGTGAGGCAGCTTTATTTTGATCA | |
| | GGAGGAGATCCCACACTCTGTTGCC | |
| | CTTGAGCACTATTTGGATGTCTCAA | |
| | TGATAGAATTTGAGAAAACTTTTGA | |
| | AGTGGAACTATCTGACAGCCTAAGC | |
| | ATCTTCCTGAAGGATAAGTCGATAG | |
| | CTTTGGACAAGCAAGAATGGTACAG | |
| | TGGTTTTGTCTCAGAAGTGACTCCG | |
| | AAGCACCTGCGAATGTCCCGTCATG | |
| | ATCGCAAGTCTACCAATAGGCTCCT | |
| | GTTAGCCTTCATTAACTCCCCTGAA | |
| | TTCGATGTTAAGGAAGAGCTTAAAT | |
| | ACTTGACTACGGGTGAGTACGCTAC | |
| | TGACCCAAATTTCAATGTCTCTTAC | |
| | TCACTCAAAGAGAAGGAAGTAAAGA | |
| | AAGAAGGGCGCATTTTCGCAAAAAT | |
| | GTCACAAAAGATGAGAGCATGCCAG | |
| | GTTATTTGTGAAGAATTGCTAGCAC | |
| | ATCATGTGGCTCCTTTGTTTAAAGA | |
| | GAATGGTGTTACTCAATCGGAGCTA | |
| | TCCCTGACAAAAAATTTGTTGGCTA | |
| | TTAGCCAACTGAGTTACAACTCGAT | |
| | GGCCGCTAAGGTGCGATTGCTGAGG | |
| | CCAGGGGACAAGTTCACTGCTGCAC | |
| | ACTATATGACCACAGACCTAAAGAA | |
| | GTACTGTCTCAATTGGCGGCACCAG | |
| | TCAGTCAAACTGTTCGCCAGAAGCC | |
| | TGGATCGACTGTTTGGGCTAGACCA | |
| | TGCTTTTTCTTGGATACATGTCCGT | |
| | CTCACCAACAGCACTATGTACGTTG | |
| | CTGACCCCTTCAATCCACCAGACTC | |
| | AGATGCATGCACAAACTTAGACGAC | |
| | AATAAGAACACCGGGATTTTTATTA | |
| | TAAGTGCACGAGGTGGTATAGAAGG | |
| | CCTCCAACAAAAACTATGGACTGGC | |
| | ATATCAATCGCAATTGCCCAAGCAG | |
| | CAGCAGCCCTCGAAGGCTTACGAAT | |
| | TGCTGCTACTCTGCAGGGGGATAAC | |
| | CAAGTTTTGGCGATTACAAAGGAGT | |
| | TCATGACCCCAGTCCCGGAGGATGT | |
| | AATCCATGAGCAGCTATCTGAGGCG | |
| | ATGTCCCGATACAAAAGGACTTTCA | |
| | CATACCTCAATTATTTAATGGGGCA | |
| | TCAGTTGAAGGATAAGGAAACCATC | |
| | CAATCCAGTGATTTCTTTGTGTACT | |
| | CCAAAAGAATCTTCTTCAATGGATC | |
| | AATCTTAAGTCAATGCCTCAAGAAC | |
| | TTCAGTAAACTCACTACTAATGCCA | |
| | CTACCCTTGCTGAGAACACTGTGGC | |
| | CGGCTGCAGTGACATCTCTTCATGC | |
| | ATTGCCCGTTGTGTGGAAAACGGGT | |
| | TGCCTAAGGATGCCGCATATATTCA | |
| | GAATATAATCATGACTCGGCTTCAA | |
| | CTATTGCTAGATCATTACTATTCAA | |
| | TGCATGGCGGCATAAACTCAGAATT | |
| | AGAGCAGCCAACTTTAAGTATCTCT | |
| | GTTCGAAACGCGACCTACTTACCAT | |
| | CTCAACTAGGCGGTTACAATCATTT | |
| | GAATATGACCCGACTATTCTGCCGC | |
| | AATATCGGCGACCCGCTTACCAGTT | |
| | CTTGGGCGGAGTCAAAAAGACTAAT | |
| | GGATGTTGGCCTTCTCAGTCGTAAG | |
| | TTCTTAGAGGGGATATTATGGAGAC | |
| | CCCCGGGAAGTGGGACATTTTCAAC | |
| | ACTCATGCTTGATCCGTTCGCACTT | |
| | AACATTGATTACCTGAGGCCGCCAG | |
| | AGACAATTATCCGAAAACACACCCA | |
| | AAAAGTCTTGTTGCAAGATTGCCCA | |
| | AATCCCCTATTAGCAGGTGTCGTTG | |
| | ACCCGAACTACAACCAAGAATTAGA | |
| | GCTATTAGCTCAGTTCTTGCTTGAT | |
| | CGGGAAACCGTTATCCCCAGGGCTG | |
| | CCCATGCCATCTTTGAATTGTCTGT | |
| | CTTGGGAAGGAAAAAACATATACAA | |
| | GGATTGGTAGATACTACAAAAACAA | |
| | TTATTCAGTGCTCATTGGAAAGACA | |
| | GCCATTGTCCTGGAGGAAAGTTGAG | |
| | AACATTGTTACCTACAACGCGCAGT | |
| | ATTTCCTCGGGGCCACCCAACAGGC | |
| | TGATACTAATGTCTCAGAAGGGCAG | |
| | TGGGTGATGCCAGGTAACTTCAAGA | |
| | AGCTTGTGTCCCTTGACGATTGCTC | |
| | AGTCACGTTGTCCACTGTATCGCGG | |
| | CGCATATCGTGGGCCAATCTACTGA | |
| | ACTGGAGAGCTATAGATGGTTTAGA | |
| | AACCCCGGATGTGATAGAGAGTATT | |
| | GATGGCCGCCTTGTACAATCATCCA | |
| | ATCAATGTGGCCTATGTAATCAAGG | |
| | GTTGGGATCCTACTCCTGGTTCTTC | |
| | TTGCCCTCTGGGTGTGTGTTCGACC | |
| | GTCCACAAGATTCTCGGGTAGTTCC | |
| | AAAGATGCCATACGTGGGGTCCAAA | |
| | ACAGATGAGAGACAGACTGCATCAG | |
| | TGCAAGCTATACAGGGATCCACTTG | |
| | TCACCTCAGAGCAGCATTGAGGCTT | |
| | GTATCACTCTATCTATGGGCCTATG | |
| | GAGATTCTGACATATCATGGCTAGA | |
| | AGCTGCGACACTGGCTCAAACACGG | |
| | TGCAATGTTTCTCTTGATGACTTGC | |
| | GAATCTTGAGCCCTCTCCCTTCTTC | |
| | GGCGAATTTACACCACAGATTAAAT | |
| | GACGGGGTAACACAGGTTAAATTCA | |
| | TGCCCGCCACATCGAGCCGAGTGTC | |
| | AAAGTTCGTCCAAATTTGCAATGAC | |
| | AACCAGAATCTTATCCGTGATGATG | |
| | GGAGTGTTGATTCCAATATGATTTA | |
| | TCAACAAGTTATGATATTGGGGCTT | |
| | GGAGAGATTGAATGCTTGCTAGCTG | |
| | ACCCAATCGATACAAACCCAGAACA | |
| | ATTGATTCTTCATCTACACTCTGAT | |
| | AATTCTTGCTGTCTCCGGGAGATGC | |
| | CAACGACCGGCTTTGTACCTGCTCT | |
| | AGGACTAACCCCATGTTTAACTGTC | |
| | CCAAAGCACAATCCTTACATTTATG | |
| | ATGATAGCCCAATACCCGGTGATTT | |
| | GGACCAGAGGCTCATCCAGACCAAA | |
| | TTTTTCATGGGTTCTGACAATTTGG | |
| | ATAATCTTGATATCTACCAACAGCG | |
| | GGCTTTATTGAGTAGGTGTGTGGCT | |
| | TATGATGTTATCCAATCGATATTTG | |
| | CTTGTGATGCACCAGTCTCTCAGAA | |
| | GAATGACGCAATCCTTCACACTGAC | |
| | TATCATGAGAATTGGATCTCAGAGT | |
| | TCCGATGGGGTGACCCTCGTATTAT | |
| | CCAAGTAACGGCAGGCTACGAGTTA | |
| | ATTCTGTTCCTTGCATACCAGCTTT | |
| | ATTATCTCAGAGTGAGGGGTGACCG | |
| | TGCAATCCTATGTTATATTGACAGG | |
| | ATACTCAACAGGATGGTATCTTCCA | |
| | ATCTAGGCAGTCTCATCCAGACACT | |
| | CTCTCATCCAGAGATTAGGAGGAGA | |
| | TTCTCATTGAGTGATCAAGGGTTCC | |
| | TTGTTGAAAGGGAGCTAGAGCCAGG | |
| | TAAGCCCTTGGTTAAACAAGCGGTT | |
| | ATGTTCTTGAGGGACTCGGTCCGCT | |
| | GCGCTTTAGCAACTATCAAGGCAGG | |
| | AATTGAGCCTGAGATCTCCCGAGGT | |
| | GGCTGTACTCAGGATGAGCTGAGCT | |
| | TTACTCTTAAGCACTTACTGTGTCG | |
| | GCGTCTCTGTGTAATCGCTCTCATG | |
| | CATTCAGAAGCAAAGAACTTGGTTA | |
| | AAGTTAGAAACCTTCCTGTAGAAGA | |
| | GAAAACCGCCTTACTGTACCAGATG | |
| | TTGGTCACTGAGGCCAATGCTAGGA | |
| | AATCAGGATCTGCTAGCATCATCAT | |
| | AAATCTAGTCTCGGCACCCCAGTGG | |
| | GACATTCATACACCAGCATTGTATT | |
| | TTGTATCAAAGAAAATGCTAGGGAT | |
| | GCTTAAAAGGTCAACCACACCCTTG | |
| | GATATAAGTGACCTCTCCGAGAGCC | |
| | AGAATCCCGCACTTGCAGAGCTGAA | |
| | TGATGTTCCCGGTCACATGGCAGAA | |
| | GAATTTCCCTGTTTGTTTAGTAGTT | |
| | ATAACGCCACATATGAAGACACAAT | |
| | TACTTACAATCCAATGACTGAAAAA | |
| | CTCGCCTTACACTTGGACAACAGTT | |
| | CCACCCCATCCAGAGCACTTGGTCG | |
| | TCACTACATCCTGCGGCCTCTTGGG | |
| | CTCTACTCATCCGCATGGTACCGGT | |
| | CTGCAGCACTACTAGCGTCAGGGGC | |
| | CCTAAATGGGTTGCCTGAGGGGTCG | |
| | AGCCTGTACCTAGGAGAAGGGTACG | |
| | GGACCACCATGACTCTGCTTGAGCC | |
| | CGTTGTCAAGTCTTCAACTGTTTAC | |
| | TACCATACATTGTTTGACCCAACCC | |
| | GGAATCCTTCACAGCGGAACTATAA | |
| | ACCAGAACCACGGGTATTCACGGAT | |
| | TCTATTTGGTACAAGGATGATTTCA | |
| | CACGGCCACCTGGTGGTATTATCAA | |
| | TCTGTGGGGTGAAGATATACGTCAG | |
| | AGTGATATCACACAGAAAGACACGG | |
| | TCAACTTCATACTATCTCAGATCCC | |
| | GCCAAAATCACTTAAGTTGATACAC | |
| | GTTGATATTGAGTTCTCACCAGACT | |
| | CCGATGTACGGACACTACTATCTGG | |
| | CTATTCTCATTGTGCACTATTGGCC | |
| | TACTGGCTATTGCAACCTGGAGGGC | |
| | GATTTGCAGTTAGAGTTTTCTTAAG | |
| | TGACCATATCATAGTAAACTTGGTC | |
| | ACTGCAATCCTGTCTGCTTTTGACT | |
| | CTAATCTGGTGTGCATTGCATCAGG | |
| | ATTGACACACAAGGATGATGGGGCA | |
| | GGTTATATTTGCGCAAAAAAGCTTG | |
| | CAAATGTTGAGGCTTCAAGGATCGA | |
| | GTACTACTTGAGGATGGTCCATGGT | |
| | TGTGTTGACTCATTAAAGATCCCTC | |
| | ATCAATTAGGAATCATTAAATGGGC | |
| | CGAGGGCGAGGTGTCCCAACTTACC | |
| | AGAAAGGCGGATGATGAAATAAATT | |
| | GGCGGTTAGGTGATCCAGTTACCAG | |
| | ATCATTTGATCCAGTTTCTGAGCTA | |
| | ATAATTGCACGAACAGGGGGGTCTG | |
| | TATTAATGGAATACGGGGCTTTTAC | |
| | TAACCTCAGGTGTGCGAACTTGGCA | |
| | GATACATACAAACTTCTGGCTTCAA | |
| | TTGTAGAGACCACCCTAATGGAAAT | |
| | AAGGGTTGAGCAAGATCAATTAGAA | |
| | GATAATTCGAGGAGACAAATCCAAG | |
| | TAGTTCCCGCTTTCAACACTAGATC | |
| | TGGGGGAAGGATCCGTACGCTGATT | |
| | GAGTGTGCTCAGCTGCAGATTATAG | |
| | ATGTTATTTGTGTAAACATAGATCA | |
| | CCTCTTTCCTAAACACCGACATGTT | |
| | CTTGTCACACAACTTACCTACCAGT | |
| | CAGTGTGCCTTGGGGACTTGATTGA | |
| | AGGCCCCCAAATTAAGACGTATCTA | |
| | AGGGCCAGGAAGTGGATCCAACGTC | |
| | AGGGACTCAATGAGACAGTTAACCA | |
| | TATCATCACTGGACAAGTGTCGCGG | |
| | AATAAAGCAAGGGATTTTTTCAAGA | |
| | GGCGTCTGAAGTTGGTTGGCTTTTC | |
| | ACTCTGCGGTGGTTGGAGCTACCTC | |
| | TCACTTTAGCTGTTCAGGTTGTTGA | |
| | TTATTATGAATAATCGGAGTCGGAA | |
| | TCGTAAATAGGAAGTCACAAAGTTG | |
| | TGAATAAACAATGATTGCATTAGTA | |
| | TTTAATAAAAAATATGTCTTTTATT | |
| | TCGT | |
| Avian | ACGAAAAAGAAGAATAAAAGGCAGA | SEQ ID |
| paramyxovir | AGCCTTTTAAAAGGAACCCTGGGCT | NO: 4 |
| us 4 APMV- | GTCGTAGGTGTGGGAAGGTTGTATT | |
| 4/duck/ | CCGAGTGCGCCTCCGAGGCATCTAC | |
| Hongkong/ | TCTACACCTATCACAATGGCTGGTG | |
| D3/75, | TCTTCTCCCAGTATGAGAGGTTTGT | |
| complete | GGACAATCAATCCCAAGTGTCAAGG | |
| genome | AAGGATCATCGGTCCTTAGCAGGAG | |
| Genbank: | GATGCCTTAAAGTTAACATCCCTAT | |
| FJ177514.1 | GCTTGTCACTGCATCTGAAGACCCC | |
| | ACCACTCGTTGGCAACTAGCATGCT | |
| | TATCTCTAAGGCTCCTGATCTCCAA | |
| | CTCATCAACCAGTGCTATCCGTCAG | |
| | GGGGCAATACTGACTCTCATGTCAT | |
| | TACCATCACAAAACATGAGAGCAAC | |
| | AGCAGCTATTGCTGGTTCCACAAAT | |
| | GCAGCTGTTATCAACACCATGGAAG | |
| | TCTTAAGTGTCAACGACTGGACCCC | |
| | ATCCTTCGACCCTAGGAGCGGTCTT | |
| | TCTGAGGAAGATGCTCAAGTTTTCA | |
| | GAGACATGGCAAGAGATCTGCCCCC | |
| | TCAGTTCACCTCTGGATCACCCTTC | |
| | ACATCAGCATTGGCGGAGGGGTTCA | |
| | CTCCTGAAGATACTCATGACCTGAT | |
| | GGAGGCCTTGACCAGTGTGCTGATA | |
| | CAGATCTGGATCCTGGTGGCTAAGG | |
| | CCATGACCAACATTGACGGCTCTGG | |
| | GGAGGCCAATGAAAGACGTCTTGCA | |
| | AAGTACATCCAAAAAGGACAGCTTA | |
| | ATCGTCAGTTTGCAATTGGTAATCC | |
| | TGCCCGTCTGATAATCCAACAGACA | |
| | ATCAAAAGCTCCTTAACTGTCCGTA | |
| | GGTTCTTGGTCTCTGAGCTTCGTGC | |
| | GTCACGAGGTGCAGTAAAAGAAGGA | |
| | TCCCCTTACTATGCAGCTGTTGGGG | |
| | ATATCCACGCTTACATCTTTAATGC | |
| | GGGATTGACACCATTCTTGACCACC | |
| | TTAAGATACGGGATAGGCACCAAGT | |
| | ACGCCGCTGTTGCACTCAGTGTGTT | |
| | CGCTGCAGATATTGCAAAGTTGAAG | |
| | AGCCTACTTACCCTGTACCAGGACA | |
| | AGGGTGTAGAAGCTGGATACATGGC | |
| | ACTCCTTGAGGATCCAGACTCCATG | |
| | CACTTTGCACCTGGAAACTTCCCAC | |
| | ACATGTACTCCTATGCAATGGGGGT | |
| | AGCTTCTTACCATGATCCTAGCATG | |
| | CGCCAATACCAATACGCCAGGAGGT | |
| | TCCTCAGCCGTCCTTTCTACTTACT | |
| | AGGAAGGGACATGGCCGCCAAGAAC | |
| | ACAGGCACGCTGGATGAGCAACTGG | |
| | CGAAGGAACTGCAAGTATCAGAGAG | |
| | AGATCGCGCCGCATTATCCGCTGCG | |
| | ATTCAATCAGCGATGGAGGGGGGAG | |
| | AGTCCGACGACTTCCCACTGTCGGG | |
| | ATCCATGCCGGCTCTCTCTGAGAAT | |
| | GCGCAACCAGTTACCCCCAGACCTC | |
| | AACAGTCCCAGCTCTCTCCCCCCCA | |
| | ATCATCAAACATGCCCCAATCAGCA | |
| | CCCAGGACCCCAGACTATCAACCCG | |
| | ACTTTGAACTGTAGGCTTCATCACC | |
| | GCACCAACAACAGCCCAAGAAGACC | |
| | ACCCCTCCCCCCACACATCTCACCC | |
| | AGCCACCCATAAAGACTCAGTCCCA | |
| | CGCCCCAGCATCTCCTTCATTTAAT | |
| | TAAAAACCGACCAACAGGGTGGGGA | |
| | AGGAGAGTCATTGGCTACTGCCAAT | |
| | TGTGTGCAGCAATGGATTTTACTGA | |
| | CATTGATGCTGTCAACTCATTGATC | |
| | GAATCATCATCGGCAATCATAGACT | |
| | CCATACAGCATGGAGGGCTGCAACC | |
| | AGCGGGCACCGTCGGCCTATCGCAG | |
| | ATCCCAAAAGGGATAACCAGCGCAT | |
| | TAACCAAGGCCTGGGAGGCTGAGGC | |
| | GGCAACTGCCGGTAATGGGGACACC | |
| | CAACACAAATCTGACAGTCCGGAGG | |
| | ATCATCAGGCCAACGACACAGATTC | |
| | CCCTGAAGACACAGGTACTGACCAG | |
| | ACCACCCAGGAGGCCAACATCGTTG | |
| | AGACACCCCACCCCGAGGTGCTGTC | |
| | AGCAGCCAAAGCCAGACTCAAGAGG | |
| | CCCAAAGCAGGGAGGGACACCCGCG | |
| | ACAACTCCCCTGCGCAACCCGATCA | |
| | TCTTTTAAAGGGGGGCCTCCTGAGC | |
| | CCACAACCAGCAGCATCATGGGTGC | |
| | AAAATCCACCCAGTCATGGAGGTCC | |
| | CGGCACCGCCGATCCCCGCCCATCA | |
| | CAAACTCAGGATCATTCCCCCACCG | |
| | GAGAGAAATGGCGATTGTCACCGAC | |
| | AAAGCAACCGGAGACATTGAACTGG | |
| | TGGAGTGGTGCAACCCGGGGTGCAC | |
| | AGCAGTCCGAATTGAACCCACCAGA | |
| | CTCGACTGTGTATGCGGACACTGCC | |
| | CCACCATCTGTAGCCTCTGCATGTA | |
| | TGACGACTGATCAGGTACAACTACT | |
| | AATGAAGGAGGTTGCTGACATAAAA | |
| | TCACTCCTTCAGGCGTTAGTGAGGA | |
| | ACCTCGCTGTCTTGCCCCAATTGAG | |
| | GAATGAGGTTGCAGCAATCAGAACA | |
| | TCACAGGCCATGATAGAGGGGACAC | |
| | TCAATTCGATCAAGATTCTTGACCC | |
| | TGGGAATTATCAGGAATCATCACTA | |
| | AACAGTTGGTTCAAACCTCGCCAAG | |
| | ATCACACTGTTGTTGTGTCTGGACC | |
| | AGGGAATCCATTGGCCATGCCAACC | |
| | CCAGTCCAAGACAACACCATATTCC | |
| | TGGACGAGCTAGCCAGACCTCATCC | |
| | TAGTGTGGTCAATCCTTCCCCACCC | |
| | ATCACCAACACCAATGTTGACCTTG | |
| | GCCCACAGAAGCAGGCTGCAATAGC | |
| | CTATATCTCCGCTAAATGCAAGGAT | |
| | CCGGGGAAACGAGATCAGCTATCAA | |
| | GGCTCATTGAGCGAGCAACCACCCC | |
| | AAGTGAGATCAACAAAGTTAAAAGA | |
| | CAAGCCCTTGGGCTCTAGATCACTC | |
| | GATCACCCCTCATGGTGATCACAAC | |
| | AATAATCAGAACCCTTCCGAACCAC | |
| | ATGACCAACCCAGCCCACCGCCCAC | |
| | ACCGTCCATCGACATCCCTTGCCAA | |
| | ACATCCTGCCGTAGCTGATTTATTC | |
| | AAAAGAGCTCATTTGATATGACCTG | |
| | GTAATCATAAAATAGGGTGGGGAAG | |
| | GTGCTTTGCCTGTAAGGGGGCTCCC | |
| | TCATCTTCAGACACGTGCCCGCCAT | |
| | CTCACCAACAGTGCAATGGCAGACA | |
| | TGGACACGGTGTATATCAATCTGAT | |
| | GGCAGATGACCCAACCCACCAAAAA | |
| | GAACTGCTGTCCTTTCCTCTCATCC | |
| | CTGTGACCGGTCCTGACGGGAAGAA | |
| | GGAACTCCAACACCAGATCCGGACC | |
| | CAATCCTTGCTCGCCTCAGACAAAC | |
| | AAACTGAACGGTTCATCTTCCTCAA | |
| | CACTTACGGATTCATCTATGACACC | |
| | ACACCGGACAAGACAACTTTTTCCA | |
| | CCCCAGAGCATATTAATCAGCCTAA | |
| | GAGGACGACGGTGAGTGCCGCGATG | |
| | ATGACCATTGGCCTGGTTCCCGCCA | |
| | ATATACCCCTGAACGAACTAACGGC | |
| | TACTGTGTTCAGCCTTAAAGTAAGA | |
| | GTGAGGAAAAGTGCGAGGTATCGGG | |
| | AAGTGGTCTGGTATCAATGCAATCC | |
| | AGTACCGGCCCTGCTTGCAGCCACC | |
| | AGGTTTGGTCGCCAAGGAGGTCTCG | |
| | AGTCGAGCACTGGAGTCAGTGTAAA | |
| | GGCTCCCGAGAAGATAGATTGTGAG | |
| | AAGGATTATACCTACTACCCTTATT | |
| | TCTTATCTGTGTGCTACATCGCCAC | |
| | CTCCAACCTGTTCAAGGTACCGAGG | |
| | ATGGTTGCTAATGCAACCAACAGTC | |
| | AATTATACCACCTTACCATGCAGGT | |
| | CACATTTGCCTTTCCAAAAAACATC | |
| | CCTCCAGCCAACCAGAAACTCCTGA | |
| | CACAGGTGGATGAGGGATTCGAGGG | |
| | CACTGTGGATTGCCATTTTGGGAAC | |
| | ATGCTGAAAAAGGATCGGAAAGGGA | |
| | ACATGAGGACACTGTCCCAGGCGGC | |
| | AGATAAGGTCAGACGAATGAATATT | |
| | CTTGTTGGTATCTTTGACTTGCATG | |
| | GGCCAACGCTCTTCCTGGAGTATAC | |
| | CGGGAAACTGACAAAGGCTCTGCTA | |
| | GGGTTCATGTCCACCAGCCGAACAG | |
| | CAATCATCCCCATATCTCAGCTCAA | |
| | TCCCATGCTGAGTCAACTCATGTGG | |
| | AGCAGTGATGCCCAGATAGTAAAGT | |
| | TAAGGGTTGTCATAACTACATCCAA | |
| | ACGCGGCCCGTGCGGGGGTGAGCAG | |
| | GAGTATGTGCTGGATCCCAAATTCA | |
| | CAGTTAAGAAAGAAAAGGCTCGACT | |
| | CAACCCTTTCGAGAAGGCAGCCTAA | |
| | TGATTTAATCCGCAAGATCCCAGAA | |
| | ATCAGACCACTCTATACTATCCACT | |
| | GATCACTGGAAATGTAATTGTACAG | |
| | TTGATGAATCTGTGAAGAATCAATT | |
| | AAAAAACCGGATCCTTATTAGGGTG | |
| | GGGAAGTAGTTGATTGGGTGTCTAA | |
| | ACAAAAGCATTTCTTCACACCTCCC | |
| | CGCCACGAAACAACCACAATGAGGC | |
| | TATCAAACACAATCTTGACCTTGAT | |
| | TCTCATCATACTTACCGGCTATTTG | |
| | ATAGGTGTCCACTCCACCGATGTGA | |
| | ATGAGAAACCAAAGTCCGAAGGGAT | |
| | TAGGGGTGATCTTACACCAGGTGCG | |
| | GGTATTTTCGTAACTCAAGTCCGAC | |
| | AGCTCCAGATCTACCAACAGTCTGG | |
| | GTACCATGATCTTGTCATCAGATTG | |
| | TTACCTCTTCTACCAACAGAGCTTA | |
| | ATGATTGTCAAAGGGAAGTTGTCAC | |
| | AGAGTACAATAACACTGTATCACAG | |
| | CTGTTGCAGCCTATCAAAACCAACC | |
| | TGGATACTTTGTTGGCAGATGGTAG | |
| | CACAAGGGATGTTGATATACAGCCG | |
| | CGATTCATTGGGGCAATAATAGCCA | |
| | CAGGTGCCCTGGCTGTAGCAACGGT | |
| | AGCTGAGGTAACTGCAGCTCAAGCA | |
| | CTATCTCAGTCAAAAACGAATGCTC | |
| | AAAATATTCTCAAGTTGAGAGATAG | |
| | TATTCAGGCCACCAACCAAGCAGTT | |
| | TTTGAAATTTCACAGGGACTCGAAG | |
| | CAACTGCAACCGTGCTATCAAAACT | |
| | GCAAACTGAGCTCAATGAGAATATC | |
| | ATCCCAAGTCTGAACAACTTGTCCT | |
| | GTGCTGCCATGGGGAATCGCCTTGG | |
| | TGTATCACTCTCACTCTATTTGACC | |
| | TTAATGACCACTCTATTTGGGGACC | |
| | AGATCACAAACCCAGTGCTGACGCC | |
| | AATCTCTTACAGCACCCTATCGGCA | |
| | ATGGCGGGTGGTCACATTGGTCCAG | |
| | TGATGAGTAAGATATTAGCCGGATC | |
| | TGTCACAAGTCAGTTGGGGGCAGAA | |
| | CAACTGATTGCTAGTGGCTTAATAC | |
| | AGTCACAGGTAGTAGGTTATGATTC | |
| | CCAGTATCAGCTGTTGGTTATCAGG | |
| | GTCAACCTTGTACGGATTCAGGAAG | |
| | TCCAGAATACTAGGGTTGTATCACT | |
| | AAGAACACTAGCAGTCAATAGGGAT | |
| | GGTGGACTTTACAGAGCCCAGGTGC | |
| | CACCCGAGGTAGTTGAGCGATCTGG | |
| | CATTGCAGAGCGGTTTTATGCAGAT | |
| | GATTGTGTTCTAACTACAACTGATT | |
| | ACATCTGCTCATCGATCCGATCTTC | |
| | TCGGCTTAATCCAGAGTTAGTCAAG | |
| | TGTCTCAGTGGGGCACTTGATTCAT | |
| | GCACATTTGAGAGGGAAAGTGCATT | |
| | ACTGTCAACTCCCTTCTTTGTATAC | |
| | AACAAGGCAGTCGTCGCAAATTGTA | |
| | AAGCAGCGACATGTAGATGTAATAA | |
| | ACCGCCATCTATCATTGCCCAATAC | |
| | TCTGCATCAGCTCTAGTAACCATCA | |
| | CCACCGACACTTGTGCTGACCTTGA | |
| | AATTGAGGGTTATCGTTTCAACATA | |
| | CAGACTGAATCCAACTCATGGGTTG | |
| | CACCAAACTTCACGGTCTCAACCTC | |
| | ACAAATAGTATCGGTTGATCCAATA | |
| | GACATATCCTCTGACATTGCCAAAA | |
| | TTAACAATTCTATCGAGGCTGCGCG | |
| | AGAGCAGCTGGAACTGAGCAACCAG | |
| | ATCCTTTCCCGAATCAACCCACGGA | |
| | TTGTGAACGACGAATCACTAATAGC | |
| | TATTATCGTGACAATTGTTGTGCTT | |
| | AGTCTCCTTGTAATTGGTCTTATTA | |
| | TTGTTCTCGGTGTGATGTACAAGAA | |
| | TCTTAAGAAAGTCCAACGAGCTCAA | |
| | GCTGCTATGATGATGCAGCAAATGA | |
| | GCTCATCACAGCCTGTGACCACCAA | |
| | ATTGGGGACACCCTTCTAGGTGAAT | |
| | AATCATATCAATCCATTCAATAATG | |
| | AGCGGGACATACCAATCACCAACGA | |
| | CTGTGTCACAAGGCCGGTTAGGAAT | |
| | GCACCGGATCTCTCTCCTTCCTTTT | |
| | TAATTAAAAACGGTTGAACTGAGGG | |
| | TGAGGGGGGGGGTGTGCATGGTAGG | |
| | GTGGGGAAGGTAGCCAATTCCTGCC | |
| | CATTGGGCCGACCGTACCAAGAGAA | |
| | GTCAACAGAAGTATAGATGCAGGGC | |
| | GACATGGAGGGTAGCCGTGATAACC | |
| | TCACAGTAGATGATGAATTAAAGAC | |
| | AACATGGAGGTTAGCTTATAGAGTT | |
| | GTATCCCTCCTATTGATGGTGAGTG | |
| | CCTTGATAATCTCTATAGTAATCCT | |
| | GACGAGAGATAACAGCCAAAGCATA | |
| | ATCACGGCGATCAACCAGTCGTATG | |
| | ACGCAGACTCAAAGTGGCAAACAGG | |
| | GATAGAAGGGAAAATCACCTCAATC | |
| | ATGACTGATACGCTCGATACCAGGA | |
| | ATGCAGCTCTTCTCCACATTCCACT | |
| | CCAGCTCAATACACTTGAGGCAAAC | |
| | CTGTTGTCCGCCCTCGGAGGTTACA | |
| | CGGGAATTGGCCCCGGAGATCTAGA | |
| | GCACTGTCGTTATCCGGTTCATGAC | |
| | TCCGCTTACCTGCATGGAGTCAATC | |
| | GATTACTCATCAATCAAACAGCTGA | |
| | CTACACAGCAGAAGGCCCCCTGGAT | |
| | CATGTGAACTTCATTCCGGCACCAG | |
| | TTACGACTACTGGATGCACAAGGAT | |
| | CCCATCCTTTTCTGTATCATCATCC | |
| | ATTTGGTGCTATACACACAATGTGA | |
| | TTGAAACAGGTTGCAATGACCACTC | |
| | AGGTAGTAATCAATATATCAGTATG | |
| | GGGGTGATTAAGAGGGCTGGCAACG | |
| | GCTTACCTTACTTCTCAACAGTCGT | |
| | GAGTAAGTATCTGACCGATGGGTTG | |
| | AATAGAAAAAGCTGTTCCGTAGCTG | |
| | CGGGATCCGGGCATTGTTACCTCCT | |
| | TTGTAGCCTAGTGTCAGAGCCCGAA | |
| | CCTGATGACTATGTGTCACCAGATC | |
| | CCACACCGATGAGGTTAGGGGTGCT | |
| | AACAAGGGATGGGTCTTACACTGAA | |
| | CAGGTGGTACCCGAAAGAATATTTA | |
| | AGAACATATGGAGCGCAAACTACCC | |
| | TGGGGTAGGGTCAGGTGCTATAGCA | |
| | GGAAATAAGGTGTTATTCCCATTTT | |
| | ACGGCGGAGTGAAGAATGGATCAAC | |
| | CCCTGAGGTGATGAATAGGGGAAGA | |
| | TATTACTACATCCAGGATCCAAATG | |
| | ACTATTGCCCTGACCCGCTGCAAGA | |
| | TCAGATCTTAAGGGCAGAACAATCG | |
| | TATTATCCTACTCGATTTGGTAGGA | |
| | GGATGGTAATGCAGGGAGTCCTAAC | |
| | ATGTCCAGTATCCAACAATTCAACA | |
| | ATAGCCAGCCAATGCCAATCTTACT | |
| | ATTTCAACAACTCATTAGGATTCAT | |
| | CGGGGCGGAATCTAGGATCTATTAC | |
| | CTCAATGGTAACATTTACCTTTATC | |
| | AAAGAAGCTCGAGCTGGTGGCCTCA | |
| | CCCCCAAATTTACCTACTTGATTCC | |
| | AGGATTGCAAGTCCGGGTACGCAGA | |
| | ACATTGACTCAGGCGTTAACCTCAA | |
| | GATGTTAAATGTTACTGTCATTACA | |
| | CGACCATCATCTGGCTTTTGTAATA | |
| | GTCAGTCAAGATGCCCTAATGACTG | |
| | CTTATTCGGGGTTTATTCAGATGTC | |
| | TGGCCTCTTAGCCTTACCTCAGACA | |
| | GCATATTTGCATTTACAATGTACTT | |
| | ACAAGGGAAGACGACACGTATTGAC | |
| | CCAGCTTGGGCGCTATTCTCCAATC | |
| | ATGTAATTGGGCATGAGGCTCGTTT | |
| | GTTCAACAAGGAGGTTAGTGCTGCT | |
| | TATTCTACCACCACTTGTTTTTCGG | |
| | ACACCATCCAAAACCAGGTGTATTG | |
| | TCTGAGTATACTTGAAGTCAGAAGT | |
| | GAGCTCTTGGGGGCATTCAAGATAG | |
| | TGCCATTCCTCTATCGTGTCTTATA | |
| | GGCACCTGCTTGGTCAAGAACCCTG | |
| | AGCAGCCATAAAATTAACACTTGAT | |
| | CTTCCTTAAAAACACCTATCTAAAT | |
| | TACTGTCTGAGATCCCTGATTAGTT | |
| | ACCCTTTCAATCAATCAATTAATTT | |
| | TTAATTAAAAACGGAAAAATGGGCC | |
| | TAGTTCCAAGGAAAGGATGGGACCC | |
| | ATTAGGGTGGGGAAGGATTACTTTG | |
| | TTCCTTGACTCGCACCCACGTACAC | |
| | CCAATCCCATTCCTGTCAAGAAGGA | |
| | ACCCTTCCCAAACTCACCTTGCAAT | |
| | GTCCAATCAGGCAGCTGAGATTATA | |
| | CTACCCACCTTCCATCTTTTATCAC | |
| | CCTTGATCGAGAATAAGTGCTTCTA | |
| | CTACATGCAATTACTTGGTCTCGTG | |
| | TTACCACATGATCACTGGAGATGGA | |
| | GGGCATTCGTCAATTTTACAGTGGA | |
| | TCAAGCACACCTTAAAAATCGTAAT | |
| | CCCCGCTTAATGGCCCACATCGATC | |
| | ACACTAAGGATAGACTAAGGGCTCA | |
| | TGGTGTCTTGGGTTTCCACCAGACT | |
| | CAGACAAGTGAGAGCCGTTTCCGTG | |
| | TCTTGCTCCATCCTGAAACTTTACC | |
| | TTGGCTATCAGCAATGGGAGGATGC | |
| | ATCAACCAGGTTCCCAAGGCATGGC | |
| | GGAACACTCTGAAATCTATCGAGCA | |
| | CAGTGTGAAGCAGGAGGCGACTCAA | |
| | CTGAAGTTACTCATGGAAAAAACCT | |
| | CACTAAAGCTAACAGGAGTATCTTA | |
| | CTTATTCTCCAATTGCAATCCCGGG | |
| | AAAACTGCAGCGGGAACTATGCCCG | |
| | TACTAAGTGAGATGGCATCAGAACT | |
| | CTTGTCAAATCCCATCTCCCAATTC | |
| | CAATCAACATGGGGGTGTGCTGCTT | |
| | CAGGGTGGCACCATGTAGTCAGCAT | |
| | CATGAGGCTCCAACAGTATCAAAGA | |
| | AGGACAGGTAAGGAAGAGAAAGCAA | |
| | TCACTGAAGTTCAGTATGGCTCGGA | |
| | CACCTGTCTCATTAATGCAGACTAC | |
| | ACCGTCGTTTTTTCCGCACAGGACC | |
| | GTGTCATAGCAGTCTTGCCTTTCGA | |
| | TGTTGTCCTCATGATGCAAGACCTG | |
| | CTTGAATCCCGACGGAATGTCTTGT | |
| | TCTGTGCCCGCTTTATGTATCCCAG | |
| | AAGCCAACTACATGAGAGGATAAGT | |
| | ACAATACTGGCCCTTGGAGACCAAC | |
| | TCGGGAGAAAAGCACCCCAAGTCCT | |
| | GTATGATTTCGTAGCTACCCTCGAA | |
| | TCATTTGCATACGCTGCTGTCCAAC | |
| | TTCATGACAACAACCCTATCTACGG | |
| | TGGGGCTTTCTTTGAGTTCAATATC | |
| | CAAGAACTGGAAGCTATTTTGTCCC | |
| | CTGCACTTAATAAGGATCAAGTCAA | |
| | CTTCTACATAAGTCAAGTTGTCTCA | |
| | GCATACAGTAACCTTCCCCCATCTG | |
| | AATCAGCAGAATTGCTATGCTTACT | |
| | ACGCCTGTGGGGTCATCCCTTGCTA | |
| | AACAGTCTTGATGCAGCAAAGAAAG | |
| | TCAGAGAATCTATGTGTGCTGGGAA | |
| | GGTTCTTGATTATAATGCTATTCGA | |
| | CTAGTTTTGTCTTTTTATCATACGT | |
| | TATTAATCAATGGGTATCGGAAGAA | |
| | ACATAAGGGTCGCTGGCCAAATGTG | |
| | AATCAACATTCACTACTCAACCCGA | |
| | TAGTGAAGCAGCTTTACTTTGATCA | |
| | GGAGGAGATCCCACACTCTGTTGCC | |
| | CTTGAGCACTATTTAGATATCTCGA | |
| | TGATAGAATTTGAGAAGACTTTTGA | |
| | AGTGGAACTATCTGATAGTCTAAGC | |
| | ATCTTTCTGAAGGATAAGTCGATAG | |
| | CTTTGGATAAACAAGAATGGCACAG | |
| | TGGTTTTGTCTCAGAAGTGACTCCA | |
| | AAGCACCTACGAATGTCTCGTCATG | |
| | ATCGCAAGTCTACCAATAGGCTATT | |
| | GTTAGCCTTTATTAACTCCCCTGAA | |
| | TTCGATGTTAAGGAAGAGCTTAAAT | |
| | ATTTGACTACAGGTGAGTATGCCAC | |
| | TGACCCAAATTTCAATGTCTCTTAC | |
| | TCACTGAAAGAGAAGGAAGTTAAGA | |
| | AAGAAGGGCGCATTTTCGCAAAGAT | |
| | GTCACAGAAAATGAGAGCATGCCAG | |
| | GTTATTTGTGAAGAGTTACTAGCAC | |
| | ATCATGTGGCTCCTTTGTTTAAAGA | |
| | GAATGGTGTTACACAATCGGAGCTA | |
| | TCCCTGACAAAGAATTTGTTGGCTA | |
| | TTAGCCAACTGAGTTACAACTCGAT | |
| | GGCCGCTAAGGTGCGATTGCTGAGG | |
| | CCAGGGGACAAGTTCACCGCTGCAC | |
| | ACTATATGACCACAGACCTAAAAAA | |
| | GTACTGCCTTAACTGGCGGCACCAG | |
| | TCAGTCAAATTGTTCGCCAGAAGCC | |
| | TGGATCGACTATTTGGGTTAGACCA | |
| | TGCTTTTTCTTGGATACACGTCCGT | |
| | CTCACCAATAGCACTATGTACGTTG | |
| | CTGACCCATTCAATCCACCAGACTC | |
| | AGATGCATGCACAAATTTAGACGAC | |
| | AATAAGAACACTGGGATTTTTATTA | |
| | TAAGTGCTCGAGGTGGTATAGAAGG | |
| | CCTTCAACAGAAACTATGGACTGGC | |
| | ATATCAATTGCAATCGCCCAGGCGG | |
| | CAGCAGCCCTCGAGGGCTTACGAAT | |
| | TGCTGCCACTTTGCAGGGGGATAAC | |
| | CAGGTTTTAGCGATTACGAAAGAAT | |
| | TCATGACCCCAGTCTCGGAGGATGT | |
| | AATCCACGAGCAGCTATCTGAAGCG | |
| | ATGTCGCGATACAAGAGGACTTTCA | |
| | CATACCTTAATTATTTAATGGGGCA | |
| | CCAATTGAAGGATAAAGAAACCATC | |
| | CAATCCAGTGACTTCTTCGTTTACT | |
| | CCAAAAGGATCTTCTTCAATGGGTC | |
| | AATCCTAAGTCAATGCCTCAAGAAC | |
| | TTCAGTAAACTCACTACCAATGCCA | |
| | CTACCCTTGCTGAGAACACTGTAGC | |
| | CGGCTGCAGTGACATCTCCTCATGC | |
| | ATAGCCCGTTGTGTGGAAAACGGGT | |
| | TGCCTAAGGATGCTGCATATGTTCA | |
| | GAATATAATCATGACTCGGCTTCAA | |
| | CTGTTGCTAGATCACTACTATTCTA | |
| | TGCATGGTGGCATAAACTCAGAGTT | |
| | AGAGCAGCCAACTCTAAGTATCCCT | |
| | GTCCGAAACGCAACCTATTTACCAT | |
| | CTCAATTAGGCGGTTACAATCATTT | |
| | GAATATGACCCGACTATTCTGTCGC | |
| | AATATCGGTGACCCGCTTACTAGTT | |
| | CTTGGGCAGAGTCAAAAAGACTAAT | |
| | GGATGTTGGCCTTCTCAGTCGTAAG | |
| | TTCTTAGAGGGGATATTATGGAGAC | |
| | CCCCGGGAAGTGGGACATTTTCAAC | |
| | ACTCATGCTTGATCCGTTCGCACTT | |
| | AACATTGATTACTTAAGGCCACCAG | |
| | AGACAATAATCCGAAAACACACCCA | |
| | AAAAGTCTTGTTGCAGGATTGTCCT | |
| | AATCCTCTATTAGCAGGTGTAGTTG | |
| | ACCCGAACTACAACCAGGAATTAGA | |
| | ATTATTAGCTCAGTTCCTGCTTGAT | |
| | CGGGAAACCGTTATTCCCAGGGCTG | |
| | CCCATGCCATCTTTGAACTGTCTGT | |
| | CTTGGGAAGGAAAAAACATATACAA | |
| | GGATTGGTTGATACTACAAAAACAA | |
| | TTATTCAGTGCTCATTAGAAAGACA | |
| | GCCACTGTCCTGGAGGAAAGTTGAG | |
| | AACATTGTAACCTACAATGCGCAGT | |
| | ATTTCCTCGGGGCCACCCAGCAGGT | |
| | TGACACCAATATCTCAGAAAGGCAG | |
| | TGGGTGATGCCAGGTAATTTCAAGA | |
| | AGCTTGTATCTCTTGACGATTGCTC | |
| | AGTCACGTTGTCCACTGTGTCACGG | |
| | CGCATTTCTTGGGCCAATCTACTTA | |
| | ACTGGAGGGCTATAGATGGTTTGGA | |
| | AACTCCAGATGTGATAGAGAGTATT | |
| | GATGGCCGCCTTGTGCAATCATCCA | |
| | ATCAATGCGGCCTATGTAATCAAGG | |
| | ATTGGGCTCCTACTCCTGGTTCTTC | |
| | TTGCCCTCCGGGTGTGTGTTCGACC | |
| | GTCCACAAGATTCTCGAGTGGTTCC | |
| | AAAGATGCCATACGTGGGATCCAAA | |
| | ACGGATGAGAGACAGACTGCGTCAG | |
| | TGCAGGCTATACAGGGATCCACATG | |
| | TCACCTTAGAGCAGCATTGAGACTT | |
| | GTATCACTCTACCTTTGGGCCTATG | |
| | GAGATTCTGACATATCATGGCTAGA | |
| | AGCCGCGACATTGGCTCAAACACGG | |
| | TGCAATATTTCTCTTGATGACCTGC | |
| | GGATCCTGAGCCCTCTTCCTTCCTC | |
| | GGCAAATTTACACCACAGATTGAAT | |
| | GACGGGGTAACACAAGTGAAATTCA | |
| | TGCCCGCCACATCGAGCCGGGTGTC | |
| | AAAGTTCGTCCAAATTTGCAATGAC | |
| | AACCAGAATCTTATCCGTGATGATG | |
| | GGAGTGTTGATTCCAATATGATTTA | |
| | TCAGCAGGTTATGATATTAGGGCTT | |
| | GGAGAGATTGAATGTTTGTTAGCTG | |
| | ACCCAATCGATACAAACCCAGAACA | |
| | ACTGATTCTTCACCTACACTCTGAT | |
| | AATTCTTGCTGTCTCCGGGAGATGC | |
| | CAACGACCGGTTTTGTACCTGCTTT | |
| | AGGATTGACCCCATGCTTAACTGTC | |
| | CCAAAGCACAATCCGTATATTTATG | |
| | ATGATAGCCCAATACCCGGTGATTT | |
| | GGATCAGAGGCTCATTCAAACCAAA | |
| | TTCTTTATGGGTTCTGACAATCTAG | |
| | ATAATCTTGATATCTACCAGCAGCG | |
| | AGCTTTACTGAGTCGGTGTGTGGCT | |
| | TATGACATTATCCAATCAGTATTCG | |
| | CTTGCGATGCACCAGTATCTCAGAA | |
| | GAATGATGCAATCCTTCACACTGAC | |
| | TACCATGAAAATTGGATCTCAGAGT | |
| | TCCGATGGGGTGACCCTCGCATAAT | |
| | CCAAGTAACAGCAGGTTACGAGTTA | |
| | ATTCTGTTCCTTGCATACCAGCTTT | |
| | ATTATCTCAGAGTGAGGGGTGACCG | |
| | TGCAATCCTGTGTTATATTGATAGG | |
| | ATACTCAACAGGATGGTATCTTCCA | |
| | ATCTAGGCAGTCTCATCCAGACGCT | |
| | CTCTCATCCGGAGATTAGGAGGAGA | |
| | TTTTCATTGAGTGATCAAGGGTTCC | |
| | TTGTCGAAAGGGAGCTAGAGCCAGG | |
| | TAAGCCACTGGTAAAACAAGCGGTT | |
| | ATGTTCCTAAGGGACTCAGTCCGCT | |
| | GCGCTTTAGCAACTATCAAGGCAGG | |
| | AATTGAGCCTGAGATCTCCCGAGGT | |
| | GGCTGTACCCAGGATGAGCTGAGCT | |
| | TTACCCTTAAGCACTTACTATGTCG | |
| | GCGTCTCTGTATAATTGCTCTCATG | |
| | CATTCGGAAGCAAAGAACTTGGTCA | |
| | AAGTTAGAAACCTTCCAGTAGAGGA | |
| | AAAAACCGCCTTACTATACCAGATG | |
| | TTGATCACTGAGGCCAATGCCAGGA | |
| | GATCAGGGTCTGCTAGTATCATCAT | |
| | AAGCTTAGTTTCAGCACCCCAGTGG | |
| | GACATTCATACACCAGCGTTGTATT | |
| | TTGTATCAAAGAAAATGCTGGGGAT | |
| | GCTCAAAAGGTCAACCACACCCTTG | |
| | GATATAAGTGACCTTTCTGAGAGCC | |
| | AGAACCTCACACCAACAGAATTGAA | |
| | TGATGTTCCTGGTCACATGGCAGAG | |
| | GAATTTCCCTGTTTGTTTAGCAGTT | |
| | ATAACGCTACATATGAAGACACAAT | |
| | TACTTACAATCCAATGACTGAAAAA | |
| | CTCGCAGTGCACTTGGACAATGGTT | |
| | CCACCCCTTCCAGAGCGCTTGGTCG | |
| | TCACTACATCCTGCGACCCCTTGGG | |
| | CTTTACTCGTCTGCATGGTACCGGT | |
| | CTGCAGCACTATTAGCGTCAGGGGC | |
| | CCTCAGTGGGTTGCCTGAGGGGTCA | |
| | AGCCTGTACTTGGGAGAGGGGTATG | |
| | GGACCACCATGACTCTACTTGAGCC | |
| | CGTTGTCAAGTCCTCAACTGTTTAC | |
| | TACCATACATTGTTTGACCCAACCC | |
| | GGAATCCTTCACAGCGGAACTACAA | |
| | ACCAGAACCGCGGGTATTCACTGAT | |
| | TCCATTTGGTACAAGGATGATTTCA | |
| | CACGACCACCTGGTGGCATTGTAAA | |
| | TCTATGGGGTGAAGACGTACGTCAG | |
| | AGTGATATTACACAGAAAGACACGG | |
| | TTAATTTCATATTATCTCGGGTCCC | |
| | GCCAAAATCACTCAAATTGATACAC | |
| | GTTGATATTGAGTTCTCCCCAGACT | |
| | CTGATGTACGGACGCTACTATCTGG | |
| | CTATTCCCATTGTGCACTATTGGCC | |
| | TACTGGCTACTGCAACCTGGAGGGC | |
| | GATTTGCGGTTAGAGTTTTCTTAAG | |
| | TGACCATATCATAGTCAACTTGGTC | |
| | ACTGCCATTCTGTCCGCTTTTGACT | |
| | CTAATCTGGTGTGCATTGCGTCAGG | |
| | ATTGACACACAAGGATGATGGGGCA | |
| | GGTTATATTTGTGCAAAGAAGCTTG | |
| | CAAATGTTGAGGCTTCAAGAATTGA | |
| | GTATTACTTGAGGATGGTCCACGGC | |
| | TGTGTTGACTCATTAAAAATTCCTC | |
| | ATCAATTAGGAATCATTAAATGGGC | |
| | TGAGGGTGAAGTGTCCCGACTTACC | |
| | AAAAAGGCGGATGATGAAATAAACT | |
| | GGCGGTTAGGTGATCCAGTTACCAG | |
| | ATCATTTGATCCGGTTTCTGAGCTA | |
| | ATAATTGCGCGAACAGGGGGATCAG | |
| | TATTAATGGAATACGGGACTTTTAC | |
| | TAACCTCAGGTGTGCGAACTTGGCA | |
| | GATACATATAAACTTTTGGCTTCAA | |
| | TTGTAGAGACCACCTTAATGGAAAT | |
| | AAGGGTTGAGCAAGATCAGTTGGAA | |
| | GATGATTCGAGGAGACAAATCCAGG | |
| | TAGTCCCTGCTTTTAATACAAGATC | |
| | CGGGGGAAGGATCCGTACATTGATT | |
| | GAGTGTGCTCAGCTGCAGGTCATAG | |
| | ATGTTATCTGTGTGAACATAGATCA | |
| | CCTCTTTCCCAAACACCGACATGCT | |
| | CTTGTCACACAACTTACTTACCAGT | |
| | CAGTGTGCCTTGGGGACTTGATTGA | |
| | AGGCCCCCAAATTAAGACATATCTA | |
| | AGGGCCAGGAAGTGGATCCAACGTA | |
| | GGGGACTCAATGAGACAATTAACCA | |
| | TATCATCACTGGACAAGTGTCGCGG | |
| | AATAAGGCAAGGGATTTTTTCAAGA | |
| | GGCGCCTGAAGTTGGTTGGCTTTTC | |
| | GCTCTGTGGCGGTTGGGGCTACCTC | |
| | TCACTTTAGCTGCTTAGATTGTTGA | |
| | TTATTATGAATAATCGGAGTCGAAA | |
| | TCGTAAATAGAAAGACATAAAATTG | |
| | CAAATAAGCAATGATCGTATTAATA | |
| | TTTAATAAAAAATATGTCTTTTATT | |
| | TCGT | |
| Avian | ACGAAAAAGAAGAATAAAAGGCAGA | SEQ ID |
| paramyxovir | AGCCTTTTAAAAGGAACCCTGGGCT | No: 5 |
| us 4 isolate | GTCGTAGGTGTGGGAAGGTTGTATT | |
| Uria_ | CCGAGTGCGCCTCCGAGGCATCTAC | |
| aalge/ | TCTACACCTATCACAATGGCTGGTG | |
| Russia/ | TCTTCTCCCAGTATGAGAGGTTTGT | |
| Tyuleniy_ | GGATAACCAATCCCAAGTGTCAAGG | |
| Island/115/ | AAGGATCATCGGTCCCTGGCAGGGG | |
| 2015, genome | GATGCCTCAAAGTCAACATCCCTAT | |
| Genbank: | GCTTGTCACTGCATCTGAAGATCCC | |
| KU601399.1 | ACCACTCGTTGGCAACTAGCATGTT | |
| | TATCTTTAAGGCTCTTGATCTCCAA | |
| | CTCATCAACCAGCGCTATCCGCCAG | |
| | GGGGCAATACTGACTCTCATGTCAC | |
| | TACCATCACAAAATATGAGAGCAAC | |
| | GGCAGCTATTGCTGGTTCCACAAAT | |
| | GCAGCTGTTATCAACACTATGGAAG | |
| | TCCTAAGTGTCAACGACTGGACCCC | |
| | ATCCTTCGACCCTAGGAGCGGTCTC | |
| | TCTGAAGAGGATGCTCAGGTTTTTA | |
| | GAGACATGGCAAGGGATCTGCCCCC | |
| | TCAGTTCACCTCCGGATCACCCTTT | |
| | ACATCAGCTTTGGCGGAGGGGTTTA | |
| | CCCCAGAAGACACCCACGACCTAAT | |
| | GGAGGCCTTGACCAGTGTGCTGATA | |
| | CAGATCTGGATCCTGGTGGCTAAGG | |
| | CCATGACCAACATTGATGGTTCTGG | |
| | GGAGGCCAATGAGAGACGTCTTGCA | |
| | AAGTATATCCAGAAGGGACAGCTCA | |
| | ATCGCCAGTTTGCAATTGGTAATCC | |
| | TGCTCGTCTAATAATCCAACAGACG | |
| | ATCAAAAGCTCCTTAACTGTCCGCA | |
| | GGTTCTTGGTCTCTGAGCTTCGTGC | |
| | ATCACGAGGTGCGGTGAAAGAAGGA | |
| | TCCCCTTATTATGCAGCTGTTGGGG | |
| | ATATCCACGCATACATCTTTAACGC | |
| | AGGACTGACACCATTCTTGACTACT | |
| | TTAAGATATGGGATCGGCACCAAGT | |
| | ATGCTGCTGTTGCACTCAGTGTGTT | |
| | CGCTGCAGACATTGCAAAATTAAAG | |
| | AGTCTACTTACCTTATACCAAGATA | |
| | AGGGTGTGGAGGCCGGATACATGGC | |
| | ACTCCTTGAAGATCCAGACTCCATG | |
| | CACTTTGCACCTGGAAACTTCCCAC | |
| | ACATGTACTCCTACGCGATGGGGGT | |
| | GGCTTCTTACCATGACCCCAGCATG | |
| | CGCCAGTACCAATATGCCAGGAGGT | |
| | TCCTCAGCCGACCCTTCTACTTGCT | |
| | AGGAAGGGACATGGCCGCCAAGAAT | |
| | ACAGGCACGCTGGATGAGCAACTGG | |
| | CAAAGGAACTGCAAGTGTCAGAGAG | |
| | AGACCGCGCCGCACTGTCCGCTGCG | |
| | ATTCAATCAGCAATGGAAGGGGGAG | |
| | AATCCGACGACTTCCCACTGTCGGG | |
| | ATCCATGCCGGCTCTCTCCGACAAT | |
| | GCACAACCAGTTACCCCAAGAACCC | |
| | AACAGTCCCAGCTCTCCCCTCCCCA | |
| | ATCATCAAGCATGTCTCAATCAGCG | |
| | CCCAGGACCCCGGACTACCAGCCTG | |
| | ATTTTGAACTGTAGGCTGCATCCAT | |
| | GCACCAGCAGCAGGCCAAAGAAACC | |
| | ACCCTCCTCTCCACACATCCCACCC | |
| | AATCACCCGCTGAGACTCAATCCAA | |
| | CACCCTAGCATCCCCCTCATTTAAT | |
| | TAAAAACTGACCAATAGGGTGGGGA | |
| | AGGAGAGTTATTGGCTATTGCCAAG | |
| | TTCGTGCAGCAATGGATTTTACCGA | |
| | TATTGATGCTGTCAACTCATTAATC | |
| | GAATCATCATCAGCAATCATAGATT | |
| | CCATACAGCATGGAGGGCTGCAACC | |
| | ATCAGGCACTGTCGGCCTATCGCAA | |
| | ATCCCAAAGGGGATAACCAGCGCTT | |
| | TAACCAAAGCCTGGGAGGCTGAGGC | |
| | AGCAAATGCTGGCAATGGGGACACC | |
| | CAACAAAAGTCTGACAGTCTGGAGG | |
| | ATCATCAGGCCAACGACACAGACTC | |
| | CCCCGAAGACACAGGCACTAACCAG | |
| | ACCATCCAGGAAACCAATATCGTTG | |
| | AAACACCCCACCCCGAAGTGCTATC | |
| | GGCAGCCAAAGCCAGACTCAAGAGG | |
| | CCCAAGGCAGGGAAGGACACCCACG | |
| | ACAATCCCTCTGCGCAACCTGATCA | |
| | TCTTTTAAAGGGGGGCCCCTTGAGC | |
| | CCACAACCAGTGGCACCGTGGGTGC | |
| | AAAATCCGCCCATTCATGGAGGTCC | |
| | CGGCACCGCCGATCCCCGCCCATCA | |
| | CAAACTCAGGATCATTCCCTCACCG | |
| | GAGAGAGATGGCAATCGTCACCGAC | |
| | AAAGCAACCGGAGCCATCGAACTGG | |
| | TGGAATGGTGCAACCCGGGGTGCAC | |
| | AGCAATCCGAATTGAACCTACCAGA | |
| | CTCGACTGTGTATGCGGACACTGCC | |
| | CCACCATCTGCAGCCTCTGCATGTA | |
| | TGACGACTGATCAGGTACAACTATT | |
| | AATGAAGGAGGTTGCCGATATGAAA | |
| | TCACTCCTTCAGGCACTAGTGAGGA | |
| | ACCTAGCTGTCCTGCCTCAACTAAG | |
| | GAACGAGGTTGCAGCAATCAGGACA | |
| | TCACAGGCTATGATAGAGGGGACAC | |
| | TTAATTCAATCAAGATTCTCGACCC | |
| | TGGGAATTATCAGGAATCATCACTA | |
| | AACAGTTGGTTCAAACCACGACAAG | |
| | ATCACGCGGTTGTTGTGTCCGGACC | |
| | AGGGAATCCATTGACCATGCCAACC | |
| | CCAATCCAGGACAATACCATATTCC | |
| | TGGATGAATTGGCAAGACCTCATCC | |
| | TAGTTTGGTCAATCCGTCCCCGCCC | |
| | ACTACCAACACTAATGTTGATCTTG | |
| | GCCCACAGAAGCAGGCTGCGATAGC | |
| | TTATATCTCAGCAAAATGCAAGGAT | |
| | CAAGGGAAACGAGATCAGCTCTCAA | |
| | AGCTCATCGAGCGAGCAACCACCTT | |
| | GAGTGAGATCAACAAAGTTAAAAGA | |
| | CAGGCTCTTGGCCTCTAGATCACCC | |
| | AATCACCCCCAGTAATGAGTACAAC | |
| | AATAATCAGAACCTCCCTAAACCAC | |
| | ATGGCCAACCAAGCACACCATCCAC | |
| | ACCACCCCTTACTATCCTTTGCCAG | |
| | AAACTCCGCCGCAGCTGATTTATTC | |
| | AAAAGAAGCCACTTGGTATAACCTA | |
| | GCAACCGCAAGATAGGGTGGGGAAG | |
| | GTGCTTTGCCTGCAAGAGGGCTCCC | |
| | TCATCTTCAGACACTTACCCGCCAA | |
| | CCCACCAGTGACACAATGGCAGACA | |
| | TGGACACTGTATATATCAATCTGAT | |
| | GGCAGATGATCCAACCCACCAAAAA | |
| | GAACTGCTGTCCTTTCCCCTCATTC | |
| | CAGTGACTGGTCCCGACGGGAAAAA | |
| | GGAACTCCAACACCAGGTTCGGACT | |
| | CAATCCTTGCTCGCCTCAGACAAGC | |
| | AAACTGAGAGGTTCATCTTCCTCAA | |
| | CACTTACGGGTTTATCTATGACACT | |
| | ACACCGGACAAGACAACTTTTTCCA | |
| | CCCCAGAGCATATCAATCAGCCCAA | |
| | GAGAACGATGGTGAGTGCTGCAATG | |
| | ATGACCATCGGCCTGGTCCCCGCCA | |
| | ATATACCCTTGAACGAACTAACAGC | |
| | TACTGTGTTTGGCCTGAAGGTGAGA | |
| | GTGAGGAAGAGTGCGAGATATCGAG | |
| | AGGTGGTCTGGTATCAGTGCAACCC | |
| | TGTACCAGCCCTGCTGGCAGCCACC | |
| | AGGTTCGGTCGCCAAGGGGGTCTCG | |
| | AATCGAGCACTGGAGTCAGTGTGAA | |
| | GGCCCCTGAGAAGATAGATTGTGAG | |
| | AAGGATTATACTTACTACCCTTATT | |
| | TCCTATCTGTGTGCTACATCGCTAC | |
| | TTCCAACCTGTTCAAGGTACCAAAA | |
| | ATGGTTGCTAATGCGACCAACAGTC | |
| | AATTATACCATCTGACCATGCAGGT | |
| | CACATTTGCCTTTCCAAAAAACATC | |
| | CCCCCAGCTAACCAGAAACTCCTGA | |
| | CACAAGTGGATGAAGGATTCGAGGG | |
| | CACTGTGGACTGCCATTTTGGGAAC | |
| | ATGCTGAAAAAGGATCGGAAAGGGA | |
| | ATATGAGGACATTGTCGCAGGCGGC | |
| | AGATAAGGTCAGACGGATGAACATC | |
| | CTTGTTGGTATCTTTGACTTGCATG | |
| | GGCCGACACTCTTCCTGGAGTATAC | |
| | CGGGAAACTAACAAAAGCTCTGCTA | |
| | GGGTTCATGTCTACCAGCCGAACAG | |
| | CAATCATCCCCATATCTCAGCTCAA | |
| | TCCTATGCTGAGTCAACTCATGTGG | |
| | AGTAGTGATGCCCAGATAGTAAAAT | |
| | TAAGAGTGGTCATAACTACATCCAA | |
| | ACGCGGCCCATGCGGGGGTGAGCAG | |
| | GAGTATGTGCTGGATCCCAAATTCA | |
| | CAGTTAAAAAAGAAAAAGCCCGACT | |
| | CAATCCTTTCAAGAAGGCAGCCCAA | |
| | TGATCAAATCTGCAGGATCTCAGAA | |
| | ATCAGACCACTCTATACTATCCACT | |
| | GATTAATAGACACGTAGCTATACAG | |
| | TTGATGAACCTATGAAGAATCAATT | |
| | AGCAAACCGAATCCTTGCTAGGGTG | |
| | GGGAAGGAGTTGATTGGGTGTCTAA | |
| | ACAAAAGCACTCCTTTGCACCTCCT | |
| | CGCCACGAAACAACCATAATGAGGT | |
| | TATCACGCACAATCCTGGCCCTGAT | |
| | TCTAGGCACACTTACCGGCTATTTA | |
| | ATGGATGCCCACTCCACCACTGTGA | |
| | ACGAGAGACCAAAGTCTGAAGGGAT | |
| | TAGGGGTGATCTTATACCAGGCGCA | |
| | GGTATCTTTGTAACTCAAGTCCGAC | |
| | AACTACAGATCTACCAACAGTCTGG | |
| | GTATCATGACCTTGTCATCAGGTTA | |
| | TTACCTCTTCTACCGGCAGAACTCA | |
| | ATGATTGTCAAAGGGAAGTTGTCAC | |
| | AGAGTACAACAATACGGTATCACAG | |
| | CTGTTGCAGCCTATCAAAACCAACC | |
| | TGGATACCTTATTGGCTGATGGTGG | |
| | TACAAGGGATGCCGATATACAGCCG | |
| | CGGTTCATTGGGGCGATAATAGCCA | |
| | CAGGTGCCCTGGCGGTGGCTACGGT | |
| | AGCTGAGGTGACTGCAGCCCAAGCA | |
| | CTATCGCAGTCGAAAACGAACGCTC | |
| | AAAATATTCTCAAGTTGAGAGATAG | |
| | TATTCAGGCCACCAACCAGGCAGTT | |
| | TTTGAAATTTCACAAGGACTTGAGG | |
| | CAACTGCAACTGTGCTATCAAAACT | |
| | GCAAACTGAGCTCAATGAGAACATT | |
| | ATCCCAAGCCTGAACAACTTGTCCT | |
| | GTGCTGCTATGGGGAATCGCCTTGG | |
| | TGTATCACTATCACTCTACTTGACC | |
| | TTAATGACCACCCTATTTGGGGACC | |
| | AGATCACAAACCCAGTGCTGACACC | |
| | AATCTCCTATAGCACTCTATCGGCA | |
| | ATGGCAGGTGGTCACATTGGCCCGG | |
| | TGATGAGTAAGATATTAGCCGGATC | |
| | TGTCACAAGTCAGTTGGGGGCAGAA | |
| | CAGTTGATTGCTAGCGGCTTAATAC | |
| | AGTCACAAGTAGTGGGTTATGATTC | |
| | CCAATATCAATTATTGGTTATCAGG | |
| | GTCAATCTTGTACGGATTCAAGAGG | |
| | TCCAGAATACGAGGGTCGTATCACT | |
| | AAGAACACTAGCGGTCAATAGGGAT | |
| | GGTGGACTTTATAGAGCCCAGGTGC | |
| | CTCCTGAGGTAGTTGAACGGTCTGG | |
| | CATTGCAGAGCGATTTTACGCAGAT | |
| | GATTGCGTTCTTACTACAACTGATT | |
| | ACATTTGCTCATCGATCCGATCTTC | |
| | TCGGCTTAATCCAGAGTTAGTCAAG | |
| | TGTCTCAGTGGGGCACTTGATTCAT | |
| | GCACATTTGAGAGGGAAAGTGCATT | |
| | ATTGTCAACCCCTTTCTTTGTATAC | |
| | AACAAGGCAGTTGTCGCAAATTGTA | |
| | AAGCAGCAACATGTAGATGTAATAA | |
| | ACCGCCGTCTATTATTGCCCAATAC | |
| | TCTGCATCGGCTCTGGTCACCATCA | |
| | CCACTGACACCTGCGCCGACCTTGA | |
| | AATTGAGGGTTATCGCTTCAACATA | |
| | CAGACTGAATCCAACTCATGGGTTG | |
| | CACCAAACTTCACTGTCTCGACTTC | |
| | ACAGATTGTATCAGTTGATCCAATA | |
| | GACATCTCCTCTGACATTGCCAAAA | |
| | TCAACAGTTCCATCGAGGCTGCAAG | |
| | AGAGCAGCTGGAACTAAGCAACCAG | |
| | ATCCTCTCCCGGATTAACCCACGAA | |
| | TCGTGAATGATGAATCACTGATAGC | |
| | TATTATCGTGACAATTGTTGTGCTT | |
| | AGTCTCCTCGTAATCGGTCTGATTG | |
| | TTGTTCTCGGTGTGATGTATAAGAA | |
| | TCTTAAGAAAGTCCAACGAGCTCAA | |
| | GCTGCCATGATGATGAAGCAAATGA | |
| | GCTCATCACAGCCTGTGACCACTAA | |
| | ATTAGGGACGCCTTTCTAGGAGGAT | |
| | AATCATATTACTCTACTCAATGATG | |
| | AGCAAGACGTACCAATTATCAATGA | |
| | TTGTGTCACAAGGCCGGTTGGGAAT | |
| | GCACCGAATCTCTCCCCTTTCTTTT | |
| | TAATTAAAAACATTTGAAGTGAGGA | |
| | TAAGAGGGGGGAAGAGTATGGTAGG | |
| | GTGGGGAAGGTAGCCAATCCCTGCC | |
| | TATTAGGCTGATCGTATCAAAAGAA | |
| | CCCAACAGAAGTCTAGATACAGGGC | |
| | AACATGGAGGGCAGCCGTGATAATC | |
| | TAACAGTGGATGATGAATTAAAGAC | |
| | AACATGGAGGTTAGCTTATAGAGTT | |
| | GTGTCCCTCCTATTGATGGTGAGCG | |
| | CTTTGATAATCTCTATAGTAATCCT | |
| | GACAAGAGATAACAGCCAAAGCATA | |
| | ATCACGGCGATCAACCAGTCATCTG | |
| | ACGCAGACTCTAAGTGGCAAACGGG | |
| | AATAGAAGGGAAAATCACCTCCATT | |
| | ATGACTGATACGCTCGATACCAGAA | |
| | ATGCAGCCCTTCTCCACATTCCACT | |
| | CCAGCTCAACACGCTTGCGGCGAAC | |
| | CTATTGTCCGCCCTTGGAGGCAACA | |
| | CAGGAATTGGCCCCGGAGATCTGGA | |
| | ACACTGCCGTTACCCTGTTCATGAC | |
| | ACCGCTTACCTGCATGGAGTTAATC | |
| | GATTACTCATCAACCAGACAGCTGA | |
| | TTATACAGCAGAAGGCCCCCTAGAT | |
| | CATGTGAACTTCATACCAGCCCCGG | |
| | TTACGACCACTGGATGCACAAGGAT | |
| | ACCATCCTTTTCTGTGTCATCGTCC | |
| | ATTTGGTGCTATACACACAACGTGA | |
| | TTGAAACCGGTTGCAATGACCACTC | |
| | AGGTAGTAACCAATATATCAGCATG | |
| | GGAGTCATTAAGAGAGCAGGCAACG | |
| | GCTTACCTTACTTCTCAACAGTTGT | |
| | AAGTAAGTATCTGACTGATGGGTTG | |
| | AATAGGAAGAGCTGTTCTGTAGCTG | |
| | CCGGATCTGGGCATTGCTACCTCCT | |
| | TTGCAGCTTAGTGTCGGAGCCTGAA | |
| | CCTGATGACTATGTATCACCTGATC | |
| | CCACACCGATGAGGTTAGGGGTGCT | |
| | AACGTGGGATGGGTCTTACACTGAA | |
| | CAGGTGGTACCCGAAAGAATATTCA | |
| | AGAACATATGGAGTGCAAACTACCC | |
| | GGGAGTAGGGTCAGGTGCTATAGTA | |
| | GGAAATAAAGTGTTATTCCCATTTT | |
| | ACGGCGGAGTGAGGAATGGATCGAC | |
| | CCCGGAGGTGATGAATAGGGGAAGA | |
| | TACTACTACATCCAGGATCCAAATG | |
| | ACTATTGCCCTGACCCGCTGCAAGA | |
| | TCAGATCTTAAGAGCGGAACAATCG | |
| | TATTACCCAACTCGATTCGGTAGGA | |
| | GGATGGTAATGCAAGGGGTCCTAGC | |
| | ATGTCCAGTATCCAACAATTCAACA | |
| | ATAGCAAGCCAATGTCAATCTTACT | |
| | ATTTTAATAACTCATTAGGGTTCAT | |
| | CGGGGCAGAATCTAGAATCTATTAT | |
| | CTCAATGGTAACATTTATCTTTATC | |
| | AGAGAAGCTCGAGTTGGTGGCCTCA | |
| | CCCCCAAATCTACCTGCTTGATTCT | |
| | AGAATTGCAAGTCCGGGTACTCAGA | |
| | CCATTGACTCAGGTGTCAATCTCAA | |
| | AATGTTAAATGTCACTGTGATTACA | |
| | CGACCATCATCTGGTTTTTGTAATA | |
| | GTCAGTCACGATGCCCTAATGATTG | |
| | CTTATTCGGGGTCTATTCGGATATC | |
| | TGGCCTCTTAGCCTTACCTCAGATA | |
| | GCATATTCGCATTCACAATGTATTT | |
| | ACAGGGGAAGACAACACGTATTGAC | |
| | CCGGCTTGGGCGCTATTCTCCAATC | |
| | ATGCAATTGGGCATGAGGCTCGTCT | |
| | GTTTAATAAGGAAGTTAGTGCTGCT | |
| | TATTCTACCACCACTTGTTTTTCGG | |
| | ACACCATCCAAAATCAGGTGTATTG | |
| | CCTGAGTATACTTGAGGTCAGAAGT | |
| | GAGCTCTTGGGAGCATTCAAAATAG | |
| | TACCATTCCTCTACCGCGTCTTGTA | |
| | GGCATCCATTCAGCCAAAAAACTTG | |
| | AGTGACCATGAGATTGACACCTGAT | |
| | CCCCCTCAAAGACACCTATCTAAAT | |
| | TACTGTTCTAGACCCATGATTAGGT | |
| | ACCTTCTTAATCAATCATTTGGTTT | |
| | TTAATTAAAAATGGAAAAATGGACC | |
| | TAGTTCCAAGAGAGGGCTGGAACCC | |
| | ATTAGGGTGGGGAAGGATTGCTTTG | |
| | CTCCTTGACTCACACTCACGTACAC | |
| | TCGATCAGACTTCTGTTAAAAAGGA | |
| | AACCTTCTCAAACTCGCCCCACGAT | |
| | GTCCAATCAGGCAGCTGAGATTATA | |
| | CTACCTAGCTTCCATCTAGAATCAC | |
| | CCTTAATCGAGAATAAGTGCTTCTA | |
| | TTATATGCAATTACTTGGTCTCGTG | |
| | TTGCCACATGATCACTGGAGATGGA | |
| | GGGCATTCGTTAACTTTACAGTGGA | |
| | TCAGGTGCACCTTAAAAATCGTAAT | |
| | CCCCGCTTAATGGCCCACATCGACT | |
| | ACACTAAAGATAGATTGAGGACTCA | |
| | TGGTGTCTTAGGTTTCCACCAGACT | |
| | CAGACAAGTTTGAGCCGTTATCGTG | |
| | TTTTGCTCCATCCTGAAACCTTACC | |
| | TTGGCTGTCAGCCATGGGAGGATGC | |
| | ATCAATCAGGTGCCTAAAGCATGGC | |
| | GGAACACCCTGAAATCGATCGAGCA | |
| | CAGTGTAAAGCAGGAGGCACCTCAA | |
| | CTAAAGCTACTCATGGAGAGAACCT | |
| | CATTAAAATTAACTGGGGTACCTTA | |
| | CTTGTTCTCTAATTGCAATCCCGGG | |
| | AAAACCAAAGCAGGAACTATACCTG | |
| | TCCTAAGTGAGATGGCATCGGAACT | |
| | CTTGTCAAATCCTATCTCCCAATTC | |
| | CAATCAACATGGGGATGTGCTGCTT | |
| | CGGGGTGGCACCATGTAGTCAGTAT | |
| | CATGAGGCTTCAGCAATATCAAAGA | |
| | AGGACAGGTAAGGAGGAAAAAGCAA | |
| | TCACTGAAGTTCAGTATGGCACAGA | |
| | CACCTGTCTCATTAACGCAGACTAC | |
| | ACCGTTGTTTTTTCCACACAGAACC | |
| | GTATCATAACGGTCTTGCCTTTCGA | |
| | TGTTGTCCTCATGATGCAAGACCTG | |
| | CTCGAATCCCGACGGAATGTCCTGT | |
| | TCTGTGCCCGCTTTATGTATCCCAG | |
| | AAGCCAACTTCATGAGAGGATAAGT | |
| | ACAATATTAGCCCTTGGAGACCAAT | |
| | TGGGGAGGAAAGCACCCCAAGTCCT | |
| | GTATGATTTTGTAGCAACCCTTGAG | |
| | TCATTTGCATACGCAGCGGTTCAAC | |
| | TTCATGACAACAATCCTACCTACGG | |
| | TGGGGCCTTCTTTGAATTCAACATC | |
| | CAAGAGTTAGAATCGATTCTGTCCC | |
| | CTGCACTTAGTAAGGATCAGGTCAA | |
| | CTTCTACATAAGTCAAGTTGTCTCA | |
| | GCGTACAGTAACCTTCCTCCATCCG | |
| | AATCGGCAGAGCTGCTGTGCCTGTT | |
| | ACGCCTGTGGGGTCATCCCTTGCTA | |
| | AACAGCCTTGATGCAGCAAAGAAAG | |
| | TCAGGGAGTCTATGTGCGCCGGGAA | |
| | GGTTCTCGATTACAACGCCATTCGA | |
| | CTTGTCTTGTCTTTTTATCATACGT | |
| | TGCTAATCAATGGGTACCGGAAGAA | |
| | ACACAAGGGTCGCTGGCCAAATGTG | |
| | AATCAACATTCACTTCTCAACCCGA | |
| | TAGTGAGGCAGCTTTATTTTGATCA | |
| | GGAGGAGATCCCACACTCTGTTGCC | |
| | CTTGAGCACTATTTGGATGTTTCAA | |
| | TGATAGAATTTGAAAAAACTTTTGA | |
| | AGTGGAACTATCTGACAGCCTAAGC | |
| | ATCTTCCTGAAGGATAAGTCGATAG | |
| | CTTTGGATAAGCAAGAATGGTATAG | |
| | TGGTTTTGTCTCAGAAGTGACTCCG | |
| | AAGCACCTGCGAATGTCCCGTCATG | |
| | ATCGCAAGTCTACCAATAGGCTCCT | |
| | GTTAGCCTTCATTAACTCCCCTGAA | |
| | TTCGATGTTAAGGAAGAGCTTAAAT | |
| | ACTTGACTACGGGTGAGTACGCCAC | |
| | TGACCCAAATTTCAATGTCTCATAC | |
| | TCACTTAAAGAGAAGGAGGTAAAGA | |
| | AAGAAGGGCGCATTTTCGCAAAAAT | |
| | GTCACAAAAGATGAGAGCGTGCCAG | |
| | GTTATTTGTGAAGAATTGCTAGCAC | |
| | ATCATGTGGCTCCTTTGTTTAAAGA | |
| | GAATGGTGTTACTCAATCAGAGCTA | |
| | TCCCTGACAAAAAATTTGTTGGCTA | |
| | TTAGCCAACTGAGTTACAACTCGAT | |
| | GGCCGCTAAGGTTCGATTGCTGCGG | |
| | CCAGGGGACAAGTTCACTGCTGCAC | |
| | ACTATATGACCACAGACCTAAAAAA | |
| | GTACTGTCTTAATTGGCGGCACCAG | |
| | TCAGTCAAACTGTTCGCCAGAAGCC | |
| | TGGATCGACTGTTTGGGTTAGACCA | |
| | TGCTTTTTCTTGGATACATGTCCGT | |
| | CTCACCAACAGCACTATGTACGTTG | |
| | CTGACCCCTTTAATCCACCAGACTC | |
| | AGATGCATGCACAAATTTAGACGAC | |
| | AATAAGAATACCGGGATCTTTATTA | |
| | TAAGTGCACGAGGTGGTATAGAAGG | |
| | CCTCCAACAAAAGCTATGGACTGGC | |
| | ATATCAATTGCAATTGCCCAAGCGG | |
| | CAGCGGCCCTCGAAGGCTTACGAAT | |
| | TGCTGCTACTCTGCAGGGGGATAAC | |
| | CAAGTTTTGGCGATTACAAAGGAAT | |
| | TCATGACCCCAGTCCCAGAAGATGT | |
| | AATCCATGAGCAGCTATCTGAGGCG | |
| | ATGTCTCGATACAAAAGGACTTTCA | |
| | CATACCTCAATTATTTAATGGGACA | |
| | TCAGTTGAAGGATAAGGAAACCATC | |
| | CAATCTAGTGATTTCTTTGTTTACT | |
| | CCAAAAGAATCTTCTTCAATGGATC | |
| | AATCTTAAGTCAATGCCTCAAGAAC | |
| | TTCAGTAAACTCACTACTAATGCCA | |
| | CTACCCTTGCTGAGAATACTGTGGC | |
| | CGGCTGCAGTGACATCTCTTCATGC | |
| | ATTGCCCGTTGTGTGGAAAACGGGT | |
| | TGCCAAAGGATGCCGCATACATCCA | |
| | GAATATAATCATGACTCGGCTTCAA | |
| | CTATTGCTAGATCATTACTATTCAA | |
| | TGCATGGCGGCATAAACTCAGAGTT | |
| | AGAGCAGCCAACGTTAAGTATCTCT | |
| | GTTCGAAACGCAACCTACTTACCAT | |
| | CTCAACTAGGCGGTTACAATCATTT | |
| | AAATATGACTCGACTATTCTGCCGC | |
| | AATATCGGCGACCCGCTTACCAGTT | |
| | CTTGGGCAGAGTCAAAAAGACTAAT | |
| | GGATGTTGGTCTCCTCAGTCGTAAG | |
| | TTCTTGGAGGGGATATTATGGAGAC | |
| | CCCCGGGAAGTGGGACGTTTTCAAC | |
| | ACTCATGCTTGATCCGTTCGCACTT | |
| | AACATTGATTACCTGAGGCCGCCAG | |
| | AGACAATTATCCGAAAACACACCCA | |
| | AAAAGTCTTATTGCAAGATTGTCCA | |
| | AACCCCCTATTAGCAGGTGTCGTTG | |
| | ACCCAAACTACAACCAAGAATTAGA | |
| | GCTGTTAGCTCAGTTCTTGCTTGAT | |
| | CGGGAAACCGTTATTCCCAGGGCTG | |
| | CCCATGCCATCTTTGAGTTGTCTGT | |
| | CTTGGGGAGGAAAAAACATATACAA | |
| | GGATTGGTAGATACTACAAAAACAA | |
| | TTATTCAGTGCTCATTGGAAAGACA | |
| | GCCATTGTCCTGGAGGAAAGTTGAG | |
| | AACATTGTTACCTACAACGCGCAGT | |
| | ATTTCCTCGGGGCCACCCAACAGGC | |
| | TGACACTAATGTCTCAGAAGGGCAG | |
| | TGGGTGATGCCAGGTAACTTCAAGA | |
| | AGCTTGTGTCCCTTGACGATTGCTC | |
| | GGTCACGTTGTCTACCGTATCACGG | |
| | CGCATATCGTGGGCCAATCTACTGA | |
| | ACTGGAGAGCTATAGACGGTTTGGA | |
| | AACCCCGGATGTGATAGAGAGTATC | |
| | GATGGCCGCCTTGTACAATCATCCA | |
| | ATCAATGTGGCCTATGTAATCAAGG | |
| | GTTGGGGTCCTACTCCTGGTTCTTC | |
| | TTGCCCTCTGGGTGTGTGTTCGACC | |
| | GTCCACAAGATTCCCGGGTGGTTCC | |
| | AAAGATGCCATATGTGGGGTCCAAA | |
| | ACAGATGAGAGACAGACTGCATCAG | |
| | TGCAAGCTATACAAGGATCCACTTG | |
| | TCACCTCAGGGCGGCATTGAGGCTT | |
| | GTATCACTCTACCTATGGGCCTATG | |
| | GGGATTCTGACATATCATGGCTAGA | |
| | AGCTGCGACACTGGCTCAAACACGG | |
| | TGCAACGTTTCTCTTGATGACTTGC | |
| | GAATCTTGAGCCCTCTCCCTTCTTC | |
| | GGCGAATTTACACCACAGATTAAAT | |
| | GACGGGGTAACACAGGTTAAATTCA | |
| | TGCCCGCCACATCGAGCCGAGTGTC | |
| | AAAGTTCGTCCAAATTTGCAATGAC | |
| | AACCAGAATCTTATCCGTGACGATG | |
| | GAAGTGTTGATTCCAATATGATTTA | |
| | TCAACAGGTTATGATATTAGGGCTT | |
| | GGGGAGATTGAATGCTTGTTAGCTG | |
| | ACCCAATTGATACAAACCCAGAACA | |
| | ATTGATTCTTCATCTACACTCTGAT | |
| | AATTCTTGCTGTCTCCGGGAGATGC | |
| | CAACGACCGGCTTTGTACCAGCTCT | |
| | AGGACTGACCCCATGTTTAACTGTC | |
| | CCAAAGCACAATCCTTACATATATG | |
| | ATGATAGCCCAATACCTGGTGATTT | |
| | GGATCAGAGGCTCATTCAGACCAAA | |
| | TTTTTCATGGGTTCTGACAATTTGG | |
| | ATAATCTTGATATCTACCAACAGCG | |
| | AGCTTTACTGAGTAGGTGTGTGGCT | |
| | TATGATGTTATCCAATCGATCTTTG | |
| | CTTGTGATGCACCAGTCTCTCAGAA | |
| | GAATGACGCAATCCTTCACACTGAC | |
| | TATCATGAGAATTGGATCTCAGAGT | |
| | TCCGATGGGGTGACCCTCGTATTAT | |
| | CCAAGTAACGGCAGGCTACGAGTTA | |
| | ATTCTGTTCCTTGCATACCAGCTTT | |
| | ATTATCTCAGAGTGAGAGGTGATCG | |
| | TGCAATCCTGTGTTATGTTGACAGG | |
| | ATACTCAATAGGATGGTATCTTCCA | |
| | ATCTAGGCAGTCTCATCCAGACACT | |
| | CTCTCATCCAGAGATTAGGAGGAGA | |
| | TTCTCGTTGAGTGATCAAGGGTTCC | |
| | TTGTTGAGAGGGAACTAGAGCCAAG | |
| | TAAGCCCTTGGTTAAACAAGCGGTT | |
| | ATGTTCTTGAGGGACTCAGTCCGCT | |
| | GCGCTCTAGCTACTATCAAGGCAGG | |
| | AATTGAGCCTGAGATCTCCCGAGGT | |
| | GGCTGTACTCAGGATGAGCTAAGCT | |
| | TTACTCTTAAGCACTTACTGTGTCG | |
| | GCGTCTCTGTGTAATCGCTCTCATG | |
| | CATTCAGAGGCAAAGAACTTGGTTA | |
| | AGGTTAGAAACCTTCCTGTAGAAGA | |
| | GAAAACCGCCTTACTGTATCAGATG | |
| | TTGGTCACTGAGGCCAATGCTAGGA | |
| | AATCAGGATCTGCTAGCATTATCAT | |
| | AAACCTAGTATCGGCACCCCAGTGG | |
| | GATATTCATACACCAGCATTGTATT | |
| | TTGTGTCAAAGAAAATGTTAGGGAT | |
| | GCTTAAGAGGTCAACCACACCCTTG | |
| | GATATAAGTGACCTCTCTGAGAGCC | |
| | AGAATCCCGCACCGGCAGAGCTGAA | |
| | TGATGTTCCTGATCACATGGCAGAA | |
| | GAATTTCCCTGTTTGTTTAGTAGTT | |
| | ATAACGCTACATATGAAGACACAAT | |
| | CACTTACAATCCAATGACTGAAAAA | |
| | CTCGCCTTGCACTTGGACAATAGTT | |
| | CCACCCCATCCAGAGCACTTGGTCG | |
| | TCACTACATCCTGCGGCCTCTTGGG | |
| | CTTTACTCATCTGCATGGTACCGGT | |
| | CTGCAGCACTACTAGCATCAGGGGC | |
| | CCTAAATGGGTTGCCTGAGGGGTCA | |
| | AGCCTGTATCTAGGAGAAGGGTACG | |
| | GGACCACCATGACTCTGCTTGAGCC | |
| | CGTTGTCAAGTCTTCAACTGTTTAC | |
| | TACCACACATTGTTTGACCCAACCC | |
| | GGAATCCTTCACAGCGGAACTATAA | |
| | ACCAGAACCACGGGTATTCACGGAT | |
| | TCTATTTGGTACAAGGATGATTTCA | |
| | CACGGCCACCTGGTGGTATTATCAA | |
| | CCTGTGGGGTGAAGATATACGTCAG | |
| | AGTGATATCACACAGAAAGACACGG | |
| | TCAACTTCATACTATCTCAGATCCC | |
| | GCCAAAGTCACTTAAGTTGATACAC | |
| | GTTGATATTGAATTCTCACCAGACT | |
| | CCGATGTACGGACACTACTTTCTGG | |
| | CTATTCTCATTGTGCATTATTGGCC | |
| | TACTGGCTATTGCAACCTGGAGGGC | |
| | GATTTGCGGTTAGGGTTTTCTTAAG | |
| | TGACCATGTCATAGTAAACTTGGTC | |
| | ACTGCAATTCTGTCTGCTTTTGACT | |
| | CTAATTTGGTGTGCATTGCATCAGG | |
| | ATTGACACACAAGGATGATGGGGCA | |
| | GGTTATATTTGCGCAAAGAAGCTTG | |
| | CAAATGTTGAGGCTTCAAGGATTGA | |
| | ATACTACCTGAGGATGGTCCATGGT | |
| | TGTGTTGACTCATTAAAGATCCCTC | |
| | ATCAATTAGGAATCATTAAATGGGC | |
| | CGAGGGTGAGGTGTCCCAACTTACC | |
| | AGAAAGGCAGATGATGAAATAAATT | |
| | GGCGGTTAGGTGATCCGGTTACCAG | |
| | ATCATTTGATCCAGTTTCTGAGCTA | |
| | ATCATTGCACGAACAGGGGGGTCTG | |
| | TATTGATGGAATACGGGGCTTTTAC | |
| | TAACCTCAGGTGTGCGAACTTGGCA | |
| | GATACATACAAACTTCTGGCTTCAA | |
| | TTGTAGAGACCACCTTAATGGAAAT | |
| | AAGGGTTGAACAAGACCAGTTGGAA | |
| | GATAATTCGAGGAGGCAAATCCAAA | |
| | TAGTCCCCGCTTTTAACACGAGATC | |
| | TGGGGGAAGGATCCGTACACTGATT | |
| | GAGTGTGCTCAGCTGCAGATTATAG | |
| | ATGTTATTTGTGTAAACATAGATCA | |
| | CCTCTTTCCTAGACACCGACATGTT | |
| | CTTGTCACGCAACTTACCTACCAGT | |
| | CGGTGTGCCTTGGGGACTTGATTGA | |
| | AGGCCCCCAAATTAAGACGTATCTG | |
| | AGGGCCAGAAAGTGGATCCAACGTC | |
| | GGGGACTCAATGAGACAGTTAACCA | |
| | TATCATCACTGGACAAGTGTCACGG | |
| | AATAAAGCAAGGGATTTTTTCAAGA | |
| | GGCGCCTGAAGTTGGTTGGCTTTTC | |
| | ACTCTGCGGTGGTTGGAGCTACCTC | |
| | TCACTTTAACTGTTCAAGTTGTTGA | |
| | TTATTATGAATAATCGGAGTCGGAA | |
| | TCGTAAATAGTAAGCCACAAAGTCG | |
| | TGAATAAACAATGATTGCATTAGTA | |
| | TTTAATAAAAAATATGTCTTTTATT | |
| | TCGT | |
| Avian | ACGAAAAAGAAGAATAAAAGGCAGA | SEQ ID |
| paramyxovir | AGCCTTTTAAAAGGAACCCTGGGCT | NO: 6 |
| us 4 isolate | GTCGTAGGTGTGGGAAGGTTGTATT | |
| APMV- | CCGAGCGCGCCTCCGAGGCATCTAC | |
| 4/Egyptian | TCTACACCTATCACAATGGCTGGTG | |
| goose/South | TCTTCTCCCAATATGAGAGGTTTGT | |
| Africa/N146 | GGACAATCAATCCCAAGTGTCAAGG | |
| 8/2010, | AAGGATCATCGGTCCCTGGCAGGGG | |
| complete | GATGCCTTAAAGTCAACATTCCTAT | |
| genome | GCTTGTCACTGCATCTGAAGATCCC | |
| Genbank: | ACCACTCGTTGGCAACTAGCGTGTT | |
| JX133079.1 | TATCTTTGAGGCTCTTGATCTCCAA | |
| | CTCATCAACCAGTGCTATCCGCCAG | |
| | GGGGCAATACTGACTCTCATGTCAC | |
| | TACCATCACAAAATATGAGAGCAAC | |
| | GGCAGCTATTGCTGGTTCCACAAAT | |
| | GCAGCTGTTATCAACACTATGGAAG | |
| | TCTTGAGTGTCAATGACTGGACCCC | |
| | ATCCTTCGACCCTAGGAGCGGTCTC | |
| | TCTGAAGAGGATGCTCAGGTTTTCA | |
| | GAGACATGGCAAAGGACCTGCCCCC | |
| | TCAGTTCACCTCCGGATCACCCTTT | |
| | ACATCAGCATTGGCGGAGGGGTTTA | |
| | CCCCAGAAGACACCACACGACCTAA | |
| | TGGAGGCCTTGACTAGTGTGCTGAT | |
| | ACAGATCTGGATCCTGGTGGCTAAG | |
| | GCCATGACCAACATTGATGGCTCTG | |
| | GAGAGGCCAATGAGAGACGTCTTGC | |
| | AAAGTACATCCAGAAGGGACAACTC | |
| | AATCGCCAGTTTGCAATTGGTAATC | |
| | CTGCTCGTCTGATAATCCAACAGAC | |
| | GATCAAAAGCTCCTTAACTGTCCGC | |
| | AGATTCTTGGTCTCTGAACTTCGTG | |
| | CATCACGAGGTGCGGTGAAAGAAGG | |
| | ATCCCCTTACTATGCAGCTGTTGGG | |
| | GACATCCACGCTTACATCTTTAACG | |
| | CAGGACTGACACCATTCTTGACTAC | |
| | CTTAAGATATGGGATCGGCACCAAG | |
| | TATGCTGCAGTTGCACTCAGTGTGT | |
| | TCGCTGCAGACATTGCAAAATTAAA | |
| | GAGCCTACTTACCCTATATCAAGAC | |
| | AAGGGTGTGGAGGCTGGATACATGG | |
| | CACTCCTTGAAGATCCAGACTCCAT | |
| | GCACTTTGCACCTGGAAACTTCCCA | |
| | CACATGTACTCCTACGCGATGGGGG | |
| | TGGCTTCTTACCATGACCCCAGCAT | |
| | GCGCCAGTACCAATATGCTAGGAGG | |
| | TTCCTCAGCCGACCTTTCTACTTGC | |
| | TAGGGAGGGACATGGCCGCCAAGAA | |
| | CACAGGCACGCTGGATGAGCAACTG | |
| | GCAAAGGAACTGCAAGTGTCAGAAA | |
| | GAGACCGCGCCGCATTGTCCGCTGC | |
| | GATTCAGTCAGCAATAGAGGGGGGA | |
| | GAATCCGACGACTTCCCACTGTCGG | |
| | GATCCATGCCGGCTCTCTCCGACAA | |
| | TGCGCAACCAGTTACCCCAAGAACC | |
| | CAACAGTCCCAGCCCTCCCCTCCCC | |
| | AATCATCAAGCATGTCTCAATCAGC | |
| | ACCCAAGACCCCGGACTACCAGCCT | |
| | GATTTTGAACTGTAGGCTGCATCAG | |
| | TGCACCAACAGCAGGCCAAAGGGAC | |
| | CACCCTCCTCCCCACACATCCCACC | |
| | CAATCACCCGCTGAGACCCAATCCA | |
| | ACACCCCAGCATCCCCCTCATTTAA | |
| | TTAAAAACTGACCAATAGGGTGGGG | |
| | AAGGAGAGCTGTTGGCTATCGCCAA | |
| | GATCGTGCAGCGATGGATTTTACCG | |
| | ATATTGATGCTGTCAACTCATTAAT | |
| | TGAATCATCATCAGCAATCATAGAT | |
| | TCCATACAGCATGGAGGGCTGCAAC | |
| | CATCAGGTACTGTTGGCCTATCGCA | |
| | AATCCCCAAGGGGATAACCAGCGCT | |
| | TTAACCAAGGCCTGGGAGGCTGAGA | |
| | CAGCAACTGCTGGCTACGGGGACAC | |
| | CCAACACAAATCTGACAGTCCGGAG | |
| | GATCATCAGGCCAACGACACAGACT | |
| | CCCCCGAAGACACAGGCACCAACCA | |
| | GACCATCCAGGAAGCCAACATCGTC | |
| | GAAACACCCCACCCCGAAGTTCTAT | |
| | CGGCAGCCAAAGCCAGACTCAAGAG | |
| | GCCCAAGGCAGGGAAGGACACCCAC | |
| | GACAATCCCCCTGCGCAACCCGATC | |
| | CCCTTTTAAAGGGGGGCCCCCTGAG | |
| | CCCACAACCAGCAGCACCGTGGGTG | |
| | CAAAATTCACCCATTCATGGAGGTC | |
| | CCGGCACCGCCGATCCCCGCCCATC | |
| | ACAAACTCAGGATCATTCCCTCACC | |
| | GGAGAGAGATGGCAATCGTCACCGA | |
| | TAAAGCAACCGGAGACATTGAACTG | |
| | GTGGAATGGTGCAACCCGGGGTGCA | |
| | CAGCAATCCGAACTGAACCAACCAG | |
| | ACTCGACTGTGTATGCGGATACTGC | |
| | CCCACCATCTGCAGCCTCTGCATGT | |
| | ATGACGACTGATCAGGTACAACTAT | |
| | TAATGAAGGAGGTTGCCGATATGAA | |
| | ATCACTCCTTCAGGCACTAGTGAGG | |
| | AACCTAGCTGTCCTGCCTCAACTAA | |
| | GGAACGAGGTTGCAGCAATCAGGAC | |
| | ATCACAGGCTATGATAGAGGGGACA | |
| | CTCAATTCAATCAAGATTCTCGACC | |
| | CTGGGAATTATCAAGAATCATCACT | |
| | GAACAGTTGGTTCAAACCACGCCAA | |
| | GATCACGCGGTTGCTGTGTCCGGAC | |
| | CAGGGAATCCATTGACCATGCCAAC | |
| | TCCAATCCAAGACAACACCATATTC | |
| | CTGGATGAACTGGCAAGACCTCATC | |
| | CTAGTTTGGTCAATCCGTCCCCGCC | |
| | CACTACCAACACTAATGTTGACCTT | |
| | GGCCCACAGAAGCAGGCTGCGATAG | |
| | CTTATATCTCAGCAAAATGCAAGGA | |
| | TCAAGGGAGACGAGATCAGCTCTCA | |
| | AAGCTCATCGAGCGAGCAACCACCT | |
| | TGAGTGAGATCAACAAAGTCAAAAG | |
| | ACAGGCCCTTGGCCTCTAGACCACT | |
| | CGACCACCCCCAGTAATGAACACAA | |
| | CAATAATCAGAACCTCCCTAAACCA | |
| | CACGGCCAACCCAGCACACCATCCA | |
| | CACCGCCCACCACTATCCCCCGCCA | |
| | AAAACTCCGCTGCAGCCGATTTATT | |
| | CAAAAGAAGCCACTTGATATGACTT | |
| | ATCAACCGCAAGGTAGGGTGGGGAA | |
| | GGTGCTTTGCCTGCAAGAGGGCTCC | |
| | CTCATCTTCAGACACGTACCCGCCA | |
| | ACCCACCAGTGACGCAATGGCAGAC | |
| | ATGGACACTGTATATATCAATCTGA | |
| | TGGCAGATGATCCAACCCACCAAAA | |
| | AGAACTGCTGTCCTTCCCTCTCATT | |
| | CCAGTGACTGGTCCCGACGGGAAAA | |
| | AGGAACTCCAACACCAGGTTCGGAC | |
| | TCAATCCTTGCTCGCCTCAGACAAG | |
| | CAAACTGAGAGGTTCATCTTCCTCA | |
| | ACACTTACGGGTTTATCTATGACAC | |
| | TACACCGGACAAGACAACTTTTTCC | |
| | ACCCCAGAGCATATCAATCAGCCCA | |
| | AGAGAACGATGGTGAGTGCTGCAAT | |
| | GATGACCATCGGCCTGGTCCCCGCC | |
| | AATATACCCTTGAACGAACTAACAG | |
| | CTACTGTGTTTGGCCTGAAAGTAAG | |
| | AGTGAGGAAGAGTGCGAGATATCGA | |
| | GAGGTGGTCTGGTATCAGTGCAACC | |
| | CTGTACCAGCCCTGCTTGCAGCCAC | |
| | CAGGTTTGGTCGCCAAGGAGGTCTC | |
| | GAATCGAGCACTGGAGTCAGTGTGA | |
| | AGGCCCCCGAGAAGATAGATTGCGA | |
| | GAAGGATTATACTTACTACCCTTAT | |
| | TTCCTATCTGTGTGCTACATCGCCA | |
| | CTTCTAACCTGTTCAAGGTACCAAA | |
| | AATGGTTGCTAATGCGACCAACAGT | |
| | CAATTATACCACCTGACGATGCAGG | |
| | TCACATTTGCCTTTCCAAAAAACAT | |
| | TCCCCCAGCTAACCAGAAACTCCTG | |
| | ACACAAGTGGATGAAGGATTCGAGG | |
| | GCACTGTGGACTGCCATTTTGGGAA | |
| | CATGCTGAAAAAGGATCGGAAAGGG | |
| | AATATGAGGACATTGTCGCAGGCGG | |
| | CAGATAAGGTCCGACGGATGAACAT | |
| | CCTTGTTGGTATCTTTGACTTGCAT | |
| | GGGCCGACACTCTTCCTGGAGTATA | |
| | CCGGGAAACTAACGAAAGCTCTGTT | |
| | AGGGTTCATGTCTACCAGCCGAACA | |
| | GCAATCATCCCCATATCTCAGCTCA | |
| | ATCCTATGCTGAGTCAACTCATGTG | |
| | GAGCAGTGATGCTCAGATAGTAAAA | |
| | TTAAGAGTGGTCATAACTACATCCA | |
| | AACGCGGCCCATGCGGGGGTGAGCA | |
| | GGAATATGTGCTGGACCCCAAATTC | |
| | ACAGTTAAAAAAGAAAAAGCCCGAC | |
| | TCAACCCTTTCAAGAAGGCAGCTTA | |
| | ATGATCAAATCTGCAGGATCTCAGG | |
| | AATCAGACCACTCTATACTATCTAC | |
| | TGATCAATAGATATGTAGCTATACA | |
| | GTTGATGAACCTATGAAGAATCAAT | |
| | TAGCAAACCGAATCCTTGCTAGGGT | |
| | GGGGAAGGAATTGATTGGGTGTCTA | |
| | AACAAAAGCACTTCTTTGCACCTAC | |
| | TCACCACAAAACAATCATAATGAGG | |
| | TTATCACGAACAATCCTGGCCCTGA | |
| | TTCTCGGCGCACTTACCGGCTATTT | |
| | AATGGATGCCCACTCCACCACTGTG | |
| | AATGAGAGACCAAAGTCTGAGGGGA | |
| | TTAGGGGTGACCTTATACCAGGTGC | |
| | AGGAATCTTTGTAACTCAAATCCGG | |
| | CAACTACAGATCTACCAACAATCTG | |
| | GGTATCATGACCTTGTCATCAGGTT | |
| | ATTACCTCTTTTACCGGCAGAACTC | |
| | AATGATTGCCAAAGGGAAGTTGTCA | |
| | CAGAGTACAACAATACAGTATCACA | |
| | GCTGTTGCAGCCTATCAAAACTAAC | |
| | CTGGATACCTTATTGGCTGATGGTG | |
| | GCACAAGGGATGCCGATATACAGCC | |
| | GCGGTTCATTGGGGCGATAATAGCC | |
| | ACAGGTGCCCTGGCAGTGGCTACGG | |
| | TAGCTGAGGTGACTGCAGCCCAAGC | |
| | ACTATCTCAGTCGAAAACGAACGCT | |
| | CAAAATATTCTCAAGTTGAGAGATA | |
| | GTATTCAGGCCACCAACCAGGCAGT | |
| | TTTTGAAATTTCACAAGGACTTGAG | |
| | GCAACTGCAACTGTACTATCAAAAC | |
| | TGCAAGCTGAGCTCAATGAGAACAT | |
| | TATCCCAAGTCTGAACAACTTGTCC | |
| | TGTGCTGCCATGGGGAATCGCCTTG | |
| | GTGTATCACTATCACTCTACTTGAC | |
| | CCTAATGACTACCCTATTTGGGGAC | |
| | CAGATCACAAACCCAGTGCTGACAC | |
| | CAATCTCCTATAGCACTTTATCGGC | |
| | AATGGCAGGTGGTCACATTGGCCCG | |
| | GTGATGAGTAAAATATTAGCCGGAT | |
| | CTGTCACAAGTCAGTTGGGGGCAGA | |
| | ACAGTTGATTGCTAGCGGCTTAATA | |
| | CAATCACAGGTAGTAGGTTATGATT | |
| | CCCAATATCAATTATTGGTTATCAG | |
| | GGTCAACCTTGTACGGATTCAAGAG | |
| | GTCCAGAATACGAGGGTCGTATCAC | |
| | TAAGAACACTAGCGGTCAATAGGGA | |
| | TGGTGGACTTTATAGAGCCCAGGTG | |
| | CCTCCCGAGGTAGTCGAACGGTCTG | |
| | GCATTGCAGAGCGATTTTATGCAGA | |
| | TGATTGTGTTCTTACTACAACTGAT | |
| | TACATTTGCTCCTCGATCCGATCTT | |
| | CTCGGCTTAATCCAGAGTTAGTCAA | |
| | ATGTCTCAGTGGGGCACTTGATTCA | |
| | TGCACATTTGAGAGGGAAAGTGCAT | |
| | TATTGTCAACCCCTTTCTTTGTATA | |
| | CAACAAGGCAGTTGTCGCAAATTGT | |
| | AAAGCGGCAACATGTAGATGCAATA | |
| | AACCGCCGTCTATTATTGCCCAATA | |
| | CTCTGCATCAGCTCTGGTCACCATC | |
| | ACCACCGACACCTGCGCCGACCTTG | |
| | AAATTGAGGGCTATCGCTTCAATAT | |
| | ACAGACTGAATCCAACTCATGGGTT | |
| | GCACCAAACTTCACTGTCTCGACTT | |
| | CACAGATTGTATCAGTTGATCCAAT | |
| | AGACATCTCCTCTGACATTGCTAAA | |
| | ATCAACAGTTCCATCGAGGCTGCAA | |
| | GAGAGCAGCTGGAACTAAGCAACCA | |
| | GATCCTTTCCCGAATTAACCCACGA | |
| | ATTGTGAATGATGAATCATTGATAG | |
| | CTATTATCGTGACAATTGTTGTGCT | |
| | TAGTCTCCTCGTAATCGGTCTGATT | |
| | GTTGTTCTCGGTGTGATGTATAAGA | |
| | ATCTTAAAAAAGTCCAACGAGCTCA | |
| | AGCTGCCATGATGATGCAGCAGATG | |
| | AGCTCATCACAGCCCGTGACCACTA | |
| | AATTAGGGACGCCCTTCTAGGATAA | |
| | TAATCATATCACTCTACTCAATGAT | |
| | GAGCAAGACGTACCAATCATCAATG | |
| | ATTGTGTCACAAGGCCGGTAGGGAA | |
| | TGCACCGAATTTCTCCCCTTTCTTT | |
| | TTAATTAAAAACATTTGTAGTGAGG | |
| | ATGAGAAGGGGAAAATGTTTGGTAG | |
| | GGTGGGGAAGGTAGCCAATTCCTGC | |
| | CTATTAGGCCGACCGTATCAAAAGA | |
| | ACTCAACAGAAGTCCAGATACAAGG | |
| | TAACATGGAGGGCAGCCGTGATAAT | |
| | CTTACAGTGGATGATGAATTAAAGA | |
| | CAACGTGGAGGTTAGCTTATAGAGT | |
| | TGTGTCCCTTCTATTGATGGTGAGC | |
| | GCTTTGATAATCTCTATAGTAATCC | |
| | TGACGAGAGATAACAGCCAAAGCGT | |
| | AATCACGGCGATCAACCAGTCATCT | |
| | GAAGCTGACTCCAAGTGGCAAACGG | |
| | GAATAGAAGGGAAAATCACCTCCAT | |
| | TATGACTGATACGCTCGATACCAGG | |
| | AATGCAGCCCTTCTCCACATTCCAC | |
| | TCCAGCTCAACTCGCTTGAGGCGAA | |
| | CCTATTGTCCGCCCTTGGGGGCAAC | |
| | ACAGGAATTGGCCCCGGAGATATAG | |
| | AGCACTGCCGTTACCCTGTTCATGA | |
| | CACCGCTTACCTGCATGGAGTTAAT | |
| | CGATTACTCATCAACCAGACAGCTG | |
| | ATTATACAGCAGAAGGCCCCCTAGA | |
| | TCATGTGAACTTCATTCCAGCCCCG | |
| | GTTACGACCACTGGATGCACAAGGA | |
| | TACCATCCTTTTCCGTGTCATCGTC | |
| | CATTTGGTGCTATACACACAACGTG | |
| | ATTGAAACCGGTTGCAATGACCACT | |
| | CAGGTAGTAACCAATATATCAGCAT | |
| | GGGAGTCATTAAGAGAGCGGGCAAC | |
| | GGCCTACCTTACTTCTCAACAGTTG | |
| | TAAGTAAGTATCTGACTGATGGGTT | |
| | GAATAGGAAAAGCTGTTCTGTAGCT | |
| | GCCGGATCTGGGCATTGCTACCTCC | |
| | TTTGCAGCTTGGTGTCGGAGCCCGA | |
| | ATCTGATGACTATGTGTCACCTGAT | |
| | CCTACACCGATGAGGTTAGGGGTGC | |
| | TAACGTGGGATGGGTCTTACACTGA | |
| | GCAGGTGGTACCCGAAAGAATATTC | |
| | AAGAACATATGGAGTGCAAACTACC | |
| | CAGGAGTAGGGTCAGGTGCTATAGT | |
| | AGGAAATAAGGTGTTATTCCCATTT | |
| | TACGGCGGAGTGAGTAATGGATCGA | |
| | CCCCGGAGGTGATGAATAGGGGAAG | |
| | ATATTACTACATCCAGGATCCAAAT | |
| | GACTATTGCCCTGACCCGCTGCAAG | |
| | ATCAGATCTTAAGGGCGGAACAATC | |
| | GTATTACCCAACTCGATTCGGTAGG | |
| | AGGATGGTGATGCAAGGGGTCCTAG | |
| | CATGTCCAGTATCCAACAATTCAAC | |
| | AATAGCAAGCCAATGTCAATCTTAC | |
| | TATTTTAATAACTCATTAGGGTTCA | |
| | TTGGGGCAGAATCTAGGATCTATTA | |
| | CCTCAATGATAACATTTATCTTTAC | |
| | CAGAGAAGCTCGAGCTGGTGGCCTC | |
| | ACCCCCAGATTTACCTGCTTGATTC | |
| | TAGGATTGCAAGTCCGGGTACTCAG | |
| | AACATTGACTCAGGTGTCAATCTCA | |
| | AGATGTTAAATGTCACTGTAATTAC | |
| | ACGACCATCATCTGGTTTTTGTAAT | |
| | AGTCAGTCACGATGCCCTAATGACT | |
| | GCTTATTCGGGGTCTACTCGGATAT | |
| | CTGGCCTCTTAGCCTTACCTCAGAT | |
| | AGCATATTCGCATTCACAATGTATT | |
| | TACAGGGGAAGACAACACGTATTGA | |
| | CCCGGCTTGGGCGCTATTCTCCAAT | |
| | CATGCGATTGGGCATGAGGCTCGTC | |
| | TGTTTAATAAGAAGGTTAGTGCTGC | |
| | TTATTCTACCACCACTTGTTTTTCG | |
| | GACACCGTCCAAAATCAGGTGTATT | |
| | GCCTGAGTATACTTGAGGTCAGGAG | |
| | TGAGCTCTTGGGAGCATTCAAAATA | |
| | GTACCATTCCTCTATCGCGTCTTGT | |
| | AGGCATCCATTCAGCCAGAAAACTT | |
| | GAGTGACCATGATATTAACACCTGA | |
| | TCCCCCTCAAAGACACCTATCTAAA | |
| | TTACTGTTCTAGACTCATGATTAGG | |
| | TACCTTCTTAATCAATCATTTGGTT | |
| | TTTAATTAAAAATGAAAAAATAGGC | |
| | CTAGTTCCAAGAGAGGGCTGGAACC | |
| | CATTAGGGTGGGGAAGGATTGCTTT | |
| | GCTCCTTGACTCACACACACGTACA | |
| | CTCGATCAGACTCCTGTTTAAAAGG | |
| | AATCCTTCTCAAACTCGCCCCACGA | |
| | TGTCCAATCAGGCGGCTGAGATTAT | |
| | ACTACCCACCTTCCATCTAGAATCA | |
| | CCCTTAATCGAAAATAAGTGCTTCT | |
| | ATTATATGCAATTACTTGGTCTCGT | |
| | GTTGCCACATGATCACTGGAGATGG | |
| | AGGGCATTCGTTAACTTTACAGTGG | |
| | ATCAGGTGCACCTTAAAAATCGTAA | |
| | TCCCCGCTTGATGGCCCACATCGAC | |
| | TACACTAAGGATAGATTAAGGACTC | |
| | ATGGTGTCTTAGGTTTCCACCAGAC | |
| | TCAGACAAGTTTGAGCCGTTATCGT | |
| | GTTTTGCTCCATCCTGAAACCTTAT | |
| | CTTGGCTATCAGCCATGGGGGGATG | |
| | CATCAATCAGGTTCCTAAAGCATGG | |
| | CGGAACACTCTGAAATCGATCGAGC | |
| | ACAGTGTAAAGCAGGAGGCACCTCA | |
| | ACTAAAGCTACTCATGGAGAGAACC | |
| | TCATTAAAATTAACTGGAGTACCTT | |
| | ACTTGTTCTCTAATTGCAATCCCGG | |
| | GAAAACCACAGCAGGTACTATGCCT | |
| | GTCCTAAGTGAGATGGCATCGGAAC | |
| | TCTTGTCGAATCCTATCTCCCAATT | |
| | CCAATCAACATGGGGGTGTGCTGCT | |
| | TCGGGGTGGCACCATGTAGTCAGTA | |
| | TCATGAGGCTCCAACAATACCAAAG | |
| | AAGGACAGGTAAAGAAGAGAAAGCG | |
| | ATCACTGAAGTTCAGTATGGCACAG | |
| | ACACCTGTCTCATTAATGCAGACTA | |
| | CACTGTTGTGTTTTCCACACAGAAC | |
| | CGTATCATAACAGTCTTGCCTTTTG | |
| | ATGTTGTCCTCATGATGCAAGACCT | |
| | GCTCGAATCCCGACGGAATGTCCTG | |
| | TTCTGTGCCCGCTTTATGTATCCCA | |
| | GAAGCCAACTTCATGAGAGGATAAG | |
| | TACAATATTAGCTCTTGGAGACCAA | |
| | CTGGGGAGAAAAGCACCCCAAGTCC | |
| | TGTATGATTTCGTAGCAACCCTTGA | |
| | GTCATTTGCATACGCGGCTGTTCAA | |
| | CTTCATGACAACAATCCTACCTACG | |
| | GTGGGGCCTTCTTTGAATTCAATAT | |
| | CCAAGAGTTAGAATCCATTCTGTCC | |
| | CCTGCACTTAGTAAGGATCAGGTCA | |
| | ACTTCTACATAAATCAAGTTGTCTC | |
| | AGCGTACAGTAACCTTCCCCCATCT | |
| | GAATCGGCAGAATTGCTGTGCCTGT | |
| | TACGCCTGTGGGGTCACCCCCTGCT | |
| | AAACAGCCTTGATGCAGCAAAGAAA | |
| | GTCAGGGAGTCTATGTGCGCCGGGA | |
| | AGGTTCTCGATTACAACGCCATTCG | |
| | ACTTGTCTTGTCTTTTTATCATACG | |
| | TTGCTAATCAACGGATACCGGAAGA | |
| | AACACAAGGGTCGCTGGCCAAATGT | |
| | GAATCAACATTCACTCCTCAACCCG | |
| | ATAGTGAGGCAGCTTTATTTTGATC | |
| | AGGAGGAGATCCCACACTCTGTTGC | |
| | TCTTGAGCACTATTTGGACGTCTCA | |
| | ATGGTAGAATTTGAAAAAACTTTTG | |
| | AAGTGGAATTATCTGACAGCCTAAG | |
| | CATCTTCCTAAAGGATAAGTCGATA | |
| | GCTTTGGATAAGCAAGAGTGGTACA | |
| | GTGGTTTTGTCTCAGAAGTGACTCC | |
| | GAAGCACCTGCGAATGTCCCGTCAT | |
| | GATCGCAAGTCTACCAATAGGCTCC | |
| | TGTTAGCCTTCATTAACTCCCCTGA | |
| | ATTCGATGTTAAGGAAGAGCTTAAA | |
| | TACTTGACTACGGGTGAGTACGCCA | |
| | CTGACCCAAATTTCAATGTCTCATA | |
| | CTCACTTAAAGAGAAGGAAGTAAAG | |
| | AAAGAGGGGCGCATTTTCGCAAAAA | |
| | TGTCACAAAAGATGAGAGCATGCCA | |
| | GGTTATTTGTGAAGAATTGCTAGCA | |
| | CATCATGTGGCTCCTTTGTTTAAAG | |
| | AGAATGGTGTTACTCAATCAGAGCT | |
| | ATCCCTGACAAAAAATTTGTTGGCT | |
| | ATTAGCCAACTGAGTTACAACTCGA | |
| | TGGCCGCTAAGGTGCGATTGCTGAG | |
| | ACCAGGGGACAAGTTCACTGCTGCA | |
| | CACTATATGACCACAGACCTAAAAA | |
| | AGTACTGTCTTAATTGGCGGCACCA | |
| | GTCAGTCAAACTGTTCGCCAGAAGC | |
| | CTGGATCGACTGTTTGGGTTAGACC | |
| | ATGCTTTTTCTTGGATACATGTCCG | |
| | CCTCACCAACAGCACTATGTACGTT | |
| | GCTGACCCCTTCAATCCACCAGACT | |
| | CAGATGCATGCATTAATTTAGACGA | |
| | CAATAAGAACACTGGGATTTTTATT | |
| | ATAAGTGCACGAGGTGGTATAGAAG | |
| | GCCTCCAACAAAAACTATGGACTGG | |
| | CATATCAATTGCAATTGCCCAAGCG | |
| | GCAGCGGCCCTCGAAGGCTTACGAA | |
| | TTGCTGCTACTCTGCAGGGGGATAA | |
| | CCAAGTTTTGGCGATTACAAAGGAA | |
| | TTCATGACCCCAGTCCCAGAGGATG | |
| | TAATCCATGAGCAGCTATCTGAGGC | |
| | GATGTCTCGATACAAAAGGACTTTC | |
| | ACATACCTCAATTATTTAATGGGAC | |
| | ATCAATTGAAGGATAAGGAAACCAT | |
| | CCAATCCAGTGATTTCTTTGTCTAT | |
| | TCCAAAAGAATCTTCTTCAATGGAT | |
| | CAATCTTAAGTCAATGCCTCAAGAA | |
| | CTTCAGTAAACTCACTACTAATGCC | |
| | ACTACCCTTGCTGAGAATACTGTGG | |
| | CCGGCTGCAGTGACATCTCTTCATG | |
| | CATTGCCCGTTGTGTGGAAAACGGG | |
| | TTGCCTAAGGATGCCGCATATATCC | |
| | AGAATATAATCATGACTCGGCTTCA | |
| | ATTATTGCTAGATCATTACTATTCA | |
| | ATGCATGGCGGCATAAACTCAGAAT | |
| | TAGAGCAGCCAACTTTAAGTATCTC | |
| | TGTTCGAAACGCAACCTACTTACCA | |
| | TCTCAACTAGGCGGTTACAATCATC | |
| | TAAATATGACCCGACTATTCTGCCG | |
| | CAATATCGGCGACCCGCTTACCAGT | |
| | TCTTGGGCGGAGTCAAAAAGACTAA | |
| | TGGATGTTGGTCTCCTCAGTCGTAA | |
| | GTTCTTGGAGGGGATATTATGGAGA | |
| | CCCCCGGGAAGTGGGACGTTTTCAA | |
| | CACTCATGCTTGACCCGTTCGCACT | |
| | TAACATTGATTACCTGAGGCCGCCA | |
| | GAAACAATTATCCGAAAACACACCC | |
| | AAAAAGTCTTGTTGCAAGATTGCCC | |
| | AAACCCCCTATTAGCAGGTGTCGTT | |
| | GACCCAAACTACAACCAAGAATTAG | |
| | AGCTGTTAGCTCAGTTCTTGCTTGA | |
| | TCGGGAGACCGTTATTCCCAGGGCT | |
| | GCCCATGCCATCTTTGAGTTGTCTG | |
| | TCTTGGGGAGGAAAAAACATATACA | |
| | AGGATTGGTGGACACTACAAAAACA | |
| | ATTATTCAGTGCTCATTGGAAAGAC | |
| | AGCCATTGTCCTGGAGGAAAGTTGA | |
| | GAACATTGTTACCTACAACGCGCAG | |
| | TATTTCCTCGGGGCCACCCAACAGG | |
| | CTGATACTAATGTCTCAGAAGGGCA | |
| | GTGGGTGATGCCAGGTAACTTCAAG | |
| | AAGCTTGTGTCCCTTGACGATTGCT | |
| | CGGTCACGTTGTCTACTGTATCACG | |
| | GCGCATATCGTGGGCCAATCTACTG | |
| | AACTGGAGAGCTATAGATGGTTTGG | |
| | AAACCCCGGATGTGATAGAGAGTAT | |
| | TGATGGCCGCCTTGTACAATCATCA | |
| | AATCAATGTGGCCTATGTAATCAAG | |
| | GGTTGGGGTCCTACTCTTGGTTCTT | |
| | CTTGCCCTCTGGGTGTGTGTTCGAC | |
| | CGTCCACAAGATTCCCGGGTAGTTC | |
| | CAAAGATGCCATACGTGGGGTCCAA | |
| | AACAGATGAGAGACAGACTGCATCA | |
| | GTGCAAGCTATACAAGGATCCACTT | |
| | GTCACCTCAGGGCAGCATTGAGGCT | |
| | TGTATCACTCTACTTATGGGCTTAT | |
| | GGAGATTCTGACATATCATGGCTAG | |
| | AAGCTGCGACACTGGCTCAAACACG | |
| | GTGCAATGTTTCTCTTGATGACTTG | |
| | CGAATCTTGAGCCCTCTCCCTTCTT | |
| | CGGCGAATTTACACCACAGATTAAA | |
| | TGACGGGGTAACACAGGTTAAATTC | |
| | ATGCCCGCCACATCGAGCCGAGTGT | |
| | CAAAGTTCGTCCAAATTTGCAATGA | |
| | CAACCAAAATCTTATCCGTGATGAT | |
| | GGGAGTGTTGATTCCAATATGATTT | |
| | ATCAACAGGTTATGATATTAGGGCT | |
| | TGGGGAGATTGAATGCTTGTTAGCT | |
| | GACCCAATTGATACAAACCCAGAAC | |
| | AATTGATTCTTCATCTACACTCTGA | |
| | TAATTCTTGCTGTCTCCGGGAGATG | |
| | CCAACGACTGGCTTTGTACCTGCTC | |
| | TAGGACTGACCCCATGTTTAACTGT | |
| | CCCAAAGCACAATCCTTACATTTAT | |
| | GATGATAGCCCAATACCTGGTGATT | |
| | TGGATCAGAGGCTCATTCAGACCAA | |
| | ATTTTTCATGGGTTCTGACAATTTG | |
| | GATAATCTTGATATCTACCAACAGC | |
| | GAGCTTTACTGAGCAGGTGTGTGGC | |
| | TTATGATGTTATCCAATCGATCTTT | |
| | GCCTGTGATGCACCAGTCTCTCAGA | |
| | AGAATGACGCAATCCTTCACACTGA | |
| | CTATCATGAGAATTGGATCTCAGAG | |
| | TTCCGATGGGGTGACCCTCGTATTA | |
| | TCCAAGTAACGGCAGGCTACGAGTT | |
| | AATTCTGTTCCTTGCATACCAGCTT | |
| | TATTATCTCAGAGTGAGGGGTGACC | |
| | GTGCAATCCTGTGTTATATTGACAG | |
| | GATACTCAATAGGATGGTATCTTCC | |
| | AATCTAGGCAGTCTCATCCAGACAC | |
| | TCTCTCATCCAGAGATTAGGAGGAG | |
| | ATTCTCATTGAGTGATCAAGGGTTC | |
| | CTTGTTGAAAGGGAATTAGAGCCAG | |
| | GTAAGCCCTTGGTTAAGCAAGCGGT | |
| | TATGTTCTTGAGGGACTCGGTCCGC | |
| | TGCGCTTTAGCAACTATCAAGGCAG | |
| | GAATTGAGCCTGAGATCTCCCGAGG | |
| | TGGCTGTACTCAGGATGAGCTGAGC | |
| | TTTACTCTTAAGCACTTACTATGCC | |
| | GGCGTCTCTGTGTAATCGCTCTCAT | |
| | GCATTCAGAAGCAAAGAACTTGGTT | |
| | AAAGTCAGAAACCTTCCTGTAGAGG | |
| | AGAAAACCGCCTTACTGTACCAAAT | |
| | GTTGGTCACTGAGGCCAATGCTAGG | |
| | AAGTCAGGATCTGCTAGCATTATCA | |
| | TAAACCTAGTCTCGGCACCCCAGTG | |
| | GGACATTCATACACCAGCACTGTAT | |
| | TTTGTGTCAAAGAAAATGCTAGGGA | |
| | TGCTTAAGAGGTCAACCACACCCTT | |
| | GGATATAAGTGACCTCTCCGAGAGC | |
| | CAGAATTCCGCACCTGCAGAGCTGA | |
| | CTGATGTTCCTGGTCACATGGCAGA | |
| | AGAGTTTCCCTGTTTGTTTAGTAGT | |
| | TATAACGCCACATATGAAGACACAA | |
| | TTACTTACAATCCAACGACTGAAAA | |
| | ACTCGCCTTGCACTTGGACAACAGT | |
| | TCCACCCCATCCAGAGCACTTGGCC | |
| | GTCACTACATCCTGCGGCCTCTTGG | |
| | GCTTTATTCATCCGCATGGTACCGG | |
| | TCTGCAGCACTACTAGCGTCAGGGG | |
| | CCTTGAATGGGTTGCCTGAGGGGTC | |
| | AAGCCTGTATCTAGGAGAAGGGTAC | |
| | GGGACCACCATGACTCTGCTTGAGC | |
| | CCGTTGTCAAGTCTTCAACTGTTTA | |
| | CTACCATACATTGTTTGACCCAACC | |
| | CGGAATCCTTCTCAGCGGAACTATA | |
| | AGCCAGAACCACGGGTATTCACGGA | |
| | TTCTATTTGGTACAAGGATGATTTC | |
| | ACACGGCCACCTGGTGGTATTATCA | |
| | ACCTGTGGGGTGAAGATATACGGCA | |
| | GAGTGATATCACACAGAAAGACACG | |
| | GTCAACTTCATACTATCTCAGATCC | |
| | CGCCAAAATCACTTAAGTTGATACA | |
| | CGTTGATATTGAATTCTCACCAGAC | |
| | TCCGATGTACGGACACTACTATCTG | |
| | GCTATTCTCATTGTGCACTATTAGC | |
| | CTACTGGCTATTGCAACCTGGAGGG | |
| | CGATTTGCAGTTAGGGTTTTCTTAA | |
| | GTGACCATATCATAGTAAACTTAGT | |
| | CACTGCAATTCTGTCTGCTTTTGAC | |
| | TCTAATTTGGTGTGCATTGCATCAG | |
| | GATTGACACACAAGGATGATGGGGC | |
| | AGGTTATATTTGCGCAAAGAAGCTT | |
| | GCAAATGTTGAGGCTTCAAGGATTG | |
| | AGCACTACTTGAGGATGGTCCATGG | |
| | TTGCGTTGACTCATTAAAGATCCCT | |
| | CATCAATTAGGAATCATTAAATGGG | |
| | CCGAGGGTGAGGTGTCCCAACTTAC | |
| | CAGAAAGGCGGATGATGAAATAAAT | |
| | TGGCGGTTAGGCGATCCTGTTACCA | |
| | GATCATTTGATCCAGTTTCTGAGCT | |
| | AATCATTGCACGAACAGGGGGGTCT | |
| | GTATTAATGGAATACGGGGCTTTTA | |
| | CTAACCTCAGGTGTGCGAACTTGGC | |
| | AGATACATACAAGCTTCTGGCTTCA | |
| | ATTGTAGAGACCACCCTAATGGAAA | |
| | TAAGGGTTGAGCAAGATCAGTTGGA | |
| | AGATAATTCGAGGAGACAAATCCAA | |
| | GTAGTCCCCGCTTTCAACACGAGAT | |
| | CTGGGGGAAGGATCCGTACGCTGAT | |
| | TGAGTGTGCTCAGCTGCAGATTATA | |
| | GATGTTATTTGTGTAAACATAGACC | |
| | ACCTCTTTCCTAAACACCGACATGT | |
| | TCTTGTCACGCAACTTACCTACCAG | |
| | TCGGTGTGCCTTGGGGACCTGATTG | |
| | AAGGCCCCCAAATTAAGACGTATCT | |
| | AAGGGCCAGAAAGTGGATCCAACGT | |
| | CAGGGACTCAATGAGACAGTTAACC | |
| | ATATCATCACTGGACAAGTGTCACG | |
| | GAATAAAGCAAGGGATTTTTTCAAG | |
| | AGGCGCTTGAAGTTGGTTGGGTTTT | |
| | CACTCTGCGGTGGTTGGAGCTACCT | |
| | CTCACTTTAGCTGTTCAGGTTGTCG | |
| | ATTATTATGAATAATCGGAGTCGGA | |
| | ATCGCAAATAGGAAGCCACAAAGTT | |
| | GTGGAGAAACAATGATTGCATTAGT | |
| | ATTTAATAAAAAATATGTCTTTTAT | |
| | TTCGT | |
| Avian | ACGAAAAAGAAGAATAAAAGGCAGA | SEQ ID |
| paramyxovir | AGCCTTTTAAAAGGAACCCTGGGCT | NO: 7 |
| us 4 strain | GTCGTAGGTGTGGGAAGGTTGTATT | |
| APMV4/ | CCGAGTGCGCCTTCGAGGCATCTAC | |
| duck/China/ | TCTACACCTATCACAATGGCTGGTG | |
| G302/2012, | TCTTCTCCCAGTATGAGAGGTTTGT | |
| complete | GGACAATCAATCCCAAGTGTCAAGG | |
| genome | AAGGATCATCGTTCCCTGGCAGGGG | |
| Genbank: | GATGCCTAAAAGTCAACATCCCTAT | |
| KC439346.1 | GCTTGTCACTGCATCTGAAGATCCC | |
| | ACCACTCGTTGGCAACTAGCATGTT | |
| | TATCCTTAAGGCTCTTGGTCTCCAA | |
| | CTCATCAACCAGTGCTATCCGCCAG | |
| | GGGGCGATACTGACTCTCATGTCAC | |
| | TACCATCACAAAATATGAGAGCAAC | |
| | GGCAGCTATTGCTGGTTCCACAAAT | |
| | GCGGCTGTTATCAACACTATGGAAG | |
| | TCTTGAGTGTCAACGACTGGACCCC | |
| | ATCCTTCGACCCCAGGAGCGGTCTC | |
| | TCTGAAGAGGATGCTCAGGTTTTCA | |
| | GAGACATGGCAAGGGACCTGCCCCC | |
| | TCAGTTCACCTCCGGGTCACCCTTT | |
| | ACATCGGCATTGGCGGAGGGGTTTA | |
| | CCCCGGAGGACACCCACGACCTAAT | |
| | GGAGGCCCTGACCAGTGTGCTGATA | |
| | CAGATCTGGATCCTGGTGGCTAAGG | |
| | CCATGACCAACATTGATGGCTCTGG | |
| | GGAAGCCAATGAGAGACGTCTTGCA | |
| | AAGTACATCCAGAAGGGACAGCTTA | |
| | ATCGCCAGTTTGCAATTGGTAATCC | |
| | TGCTCGTCTGATAATCCAACAGACG | |
| | ATCAAAAGCTCCTTAACTGTCCGCA | |
| | GGTTCTTGGTCTCTGAGCTTCGTGC | |
| | ATCACGAGGTGCGGTGAAAGAAGGA | |
| | TCCCCTTACTATGCGGCTGTTGGGG | |
| | ATATCCACGCTTACATCTTTAACGC | |
| | AGGACTGACACCATTCTTGACTACC | |
| | TTAAGATACGGGATAGGCACCAAAT | |
| | ATGCTGCTGTTGCACTCAGTGTGTT | |
| | CGCTGCAGACATTGCAAAATTAAAG | |
| | AGTCTACTTACCCTATACCAGGACA | |
| | AGGGTGTGGAGGCCGGATACATGGC | |
| | ACTCCTCGAAGATCCAGACTCTATG | |
| | CACTTTGCGCCTGGAAACTTCCCAC | |
| | ACATGTACTCCTACGCGATGGGGGT | |
| | GGCTTCTTACCATGACCCCAGCATG | |
| | CGCCAGTACCAATATGCTAGGAGGT | |
| | TCCTCAGCCGTCCTTTCTACTTGCT | |
| | AGGGAGGGACATGGCTGCCAAGAAC | |
| | ACAGGCACGCTGGATGAGCAACTGG | |
| | CAAAGGAACTACAAGTGTCAGAAAG | |
| | AGACCGTGCCGCATTGTCCGCTGCG | |
| | ATTCAATCAGCAATGGAGGGGGGAG | |
| | AATCTGACGACTTCCCACTATCGGG | |
| | ATCCATGCCGGCTCTCTCCGACAAT | |
| | GCGCAACCAGTTACCCCAAGAACTC | |
| | AACAGTCCCAGCTCTCCCCTCCCCA | |
| | ATCATCAAGCATGTCTCAATCAGCG | |
| | CCCAGGACCCCGGACTACCAGCCTG | |
| | ATTTTGAACTGTAGGCTGCATCCAC | |
| | GCACCAACAGCAGGCCAAAGAAACC | |
| | ACCCCCCTCCTCACACATCCCACCC | |
| | AATCACCCGCCAAGACCCAATCCAA | |
| | CACCCCAGCATCCCCCTCATTTAAT | |
| | TAAAAACTGACCAATAGGGTGGGGA | |
| | AGGAGAGTTATTGGCTATTGCCAAG | |
| | TTCGTGCAGCAATGGATTTTACCGA | |
| | TATTGATGCTGTCAACTCATTAATT | |
| | GAATCATCATCAGCAATCATAGATT | |
| | CCATACAGCATGGAGGGCTGCAACC | |
| | ATCAGGCACTGTCGGCCTATCACAA | |
| | ATCCCAAAGGGGATAACCAGCGCCT | |
| | TAACCAAGGCCTGGGAGGCCGAGGC | |
| | AGCAACTGCTGGCAACGGGGACACC | |
| | CAACACAAATCTGACAGTCCGGAAG | |
| | ACCATCAGGCCAACGACGCAGACTC | |
| | CCCCGAAGACACAGGCACCAACCAG | |
| | ACCATCCAAGAAGCCAATATCGTTG | |
| | AAACACCCCACCCCGAAGTGCTATC | |
| | GGCAGCCAAAGCCAGACTCAAGAGG | |
| | CCCAAGACAGGGAGGGACACCCACG | |
| | ACAATCCCTCTGCGCAACCTGATCA | |
| | TCTTTTAAAGGGGGGCCCCCTGAGC | |
| | CCACAACCAGCGGCACCGTGGGTGA | |
| | AAGATCCATCCATTCATGGAGGTCC | |
| | CGGCACCGCCGATCCCCGCCCATCA | |
| | CAAACTCAGGATCATTCCCTCACCG | |
| | GAGAGAGATGGCAATCGTCACCGAC | |
| | AAAGCAACCGGAGACATCGAACTGG | |
| | TGGAATGGTGCAACCCGGGGTGCAC | |
| | AGCTATCCGAGCTGAACCAACCAGA | |
| | CTCGACTGTGTATGCGGACACTGCC | |
| | CCACCATCTGCAGCCTCTGCATGTA | |
| | TGACGACTGATCAGGTACAACTATT | |
| | AATGAAGGAGGTTGCCGACATGAAA | |
| | TCACTCCTTCAGGCACTAGTGAGGA | |
| | ACCTAGCTGTCCTGCCTCAACTAAG | |
| | GAATGAGGTTGCAGCAATCAGGACA | |
| | TCACAGGCCATGATAGAGGGGACAC | |
| | TCAATTCAATCAAGATTCTCGACCC | |
| | TGGGAATTATCAAGAATCATCACTA | |
| | AACAGTTGGTTCAAACCACGCCAAG | |
| | ATCACGCGGTTGTTGTGTCCGGACC | |
| | AGGGAATCCATTGGCCATGCCAACC | |
| | CCGATCCAAGACAACACCATATTCC | |
| | TAGATGAACTGGCAAGACCTCATCC | |
| | TAGTTTGGTCAATCCGTCCCCGCCC | |
| | GCTACCAACACCAATGCTGATCTTG | |
| | GCCCACAGAAGCAGGCTGCGATAGC | |
| | TTATATCTCAGCAAAATGCAAGGAT | |
| | CAAGGGAAACGAGACCAGCTCTCAA | |
| | AGCTCATCGAGCGAGCAACCACCCT | |
| | GAGCGAGATCAACAAAGTCAAAAGA | |
| | CAGGCCCTTGGCCTCTAGACCACTC | |
| | GACCACCCCCAGTGATGAATACAAC | |
| | AATAATCAGAACCTCCCTAAACCAC | |
| | ATGGCCAACCCAGCGCACCATCCAC | |
| | ACCACCTATTACTACCCTTCGCCAG | |
| | AAACTCCGCCGCAGCCGATTTATTC | |
| | AAAAGAAGCCACTCGATATGACTTA | |
| | GCAACCGCAAGATAGGGTGGGGAAG | |
| | GTGCTTTACCTGCAAGAGGGCTCCC | |
| | TCATCTTCAGACACGCACCCGCCAA | |
| | CCCACCAGTGACGCAATGGCAGACA | |
| | TGGACACTGTATATATCAATCTGAT | |
| | GGCAGATGATCCAACCCACCAAAAA | |
| | GAACTGCTGTCCTTTCCCCTCATTC | |
| | CCGTGACTGGTCCTGACGGGAAAAA | |
| | GGAACTCCAACACCAGGTCCGGACT | |
| | CAATCCTTGCTCGCCTCAGACAAGC | |
| | AAACTGAGAGGTTCATCTTCCTCAA | |
| | CACTTACGGGTTTATCTATGACACT | |
| | ACACCGGACAAGACAACTTTTTCTA | |
| | CCCCAGAGCATATCAATCAACCCAA | |
| | GAGAACGATGGTGAGTGCTGCAATG | |
| | ATGACCATCGGCCTGGTCCCCGCCA | |
| | ATATACCCTTGAACGAACTAACAGC | |
| | TACTGTGTTTGGCCTGAAAATAAGA | |
| | GTGAGGAAGAGTGCGAGATATCGAG | |
| | AGGTGGTCTGGTACCAGTGCAACCC | |
| | TGTACCAGCCCTGCTTGCAGCCACA | |
| | AGGTTTGGTCGCCAAGGAGGTCTCG | |
| | AATCGAGCACTGGAGTTAGTGTAAG | |
| | GGCCCCCGAGAAGATAGACTGCGAG | |
| | AAGGATTATACTTACTACCCTTATT | |
| | TCCTATCTGTGTGCTACATCGCCAC | |
| | TTCCAACCTGTTCAAGGTACCAAAA | |
| | ATGGTCGCTAATGCGACCAACAGTC | |
| | AATTATACCACCTGACCATGCAGAT | |
| | CACATTTGCCTTTCCAAAAAACATC | |
| | CCCCCAGCTAACCAGAAACTCCTGA | |
| | CACTAGTGGATGAAGGATTCGAGGG | |
| | CACTGTGGACTGCCATTTTGGGAAC | |
| | ATGCTGAAAAAGGATCGGAAAGGGA | |
| | ACATGAGGACACTGTCGCAGGCGGC | |
| | AGACAAGGTCAGACGGATGAACATC | |
| | CTTGTTGGTATCTTTGACTTGCATG | |
| | GGCCAACACTCTTCCTGGAGTACAC | |
| | CGGGAAGCTAACAAAAGCTCTGTTA | |
| | GGGTTCATGTCTACCAGCCGAACAG | |
| | CAATCATCCCCATATCTCAGCTCAA | |
| | TCCTATGCTGAGTCAACTCATGTGG | |
| | AGCAGTGATGCCCAGATAGTAAAAT | |
| | TAAGAGTGGTCATAACTACATCCAA | |
| | ACGCGGCCCATGCGGGGGTGAGCAG | |
| | GAGTATGTGCTGGATCCCAAATTCA | |
| | CTGTTAAAAAAGAGAAAGCCCGACT | |
| | CAACCCTTTCAAGAAGGCAGCCCAA | |
| | TGATCAAATCTACAAGATCTCAGGA | |
| | ATCAGACCACTCTATACTATCCACT | |
| | GATCAATAGACATGTAGCTATACAG | |
| | TTGATGAACCTATGAAGAATCAGTT | |
| | AGAAAACCGAATCCTTACTAGGGTG | |
| | GGGAAGGAGTTGATTGGGTGTCTAA | |
| | ACAAAAACATTCCTTTACACCTCCT | |
| | CGCCACGAAACAACCATAATGAGGT | |
| | TATCACGCACAATCCTGACCTTGAT | |
| | TCTCGGCACACTTACTGATTATTTA | |
| | ATGGGTGCTCACTCCACCAATGTAA | |
| | CTGAGAGACCAAAGTCTGAGGGGAT | |
| | TAGGGGTGATCTTACACCAGGCGCA | |
| | GGTATCTTTGTAACTCAAGTCCGAC | |
| | AACTACAGATCTACCAACAGTCTGG | |
| | GTATCATGACCTTGTCATCAGATTA | |
| | TTACCTCTTCTACCGGCAGAACTCA | |
| | ATGATTGTCAAAGGGAAGTTGTCAC | |
| | AGAGTACAACAATACGGTATCACAG | |
| | CTGTTGCAGCCTATCAAAACCAACC | |
| | TGGATACCTTACTGGCTGGTGGTGG | |
| | CACAAGGGATGCCGATATACAGCCG | |
| | CGGTTCATTGGGGCAATCATAGCCA | |
| | CAGGTGCCCTGGCGGTGGCTACGGT | |
| | AGCTGAGGTGACTGCAGCCCAAGCA | |
| | CTATCTCAGTCGAAAACAAACGCTC | |
| | AAAATATTCTCAAGTTGAGGGATAG | |
| | TATTCAGGCCACCAACCAGGCAGTT | |
| | TTCGAAATTTCACAAGGACTCGAGG | |
| | CAACTGCAACTGTGCTATCAAAACT | |
| | GCAAACTGAGCTCAATGAGAACATT | |
| | ATCCCAAGCCTGAACAACTTGTCCT | |
| | GTGCTGCCATGGGTAATCGCCTTGG | |
| | TGTATCACTATCACTCTACTTGACC | |
| | TTAATGACCACCCTATTTGGGGACC | |
| | AGATCACAAACCCAGTGCTGACACC | |
| | GATCTCCTATAGCACTCTATCGGCA | |
| | ATGGCAGGTGGTCATATTGGCCCGG | |
| | TAATGAGTAAAATATTAGCCGGATC | |
| | TATCACAAGTCAGTTGGGGGCGGAA | |
| | CAGTTGATTGCTAGCGGCTTAATAC | |
| | AGTCACAGGTAGTAGGTTATGATTC | |
| | CCAATACCAATTATTGGTTATCAGG | |
| | GTCAACCTTGTACGGATTCAAGAGG | |
| | TCCAGAATACGAGAGTCGTATCACT | |
| | AAGAACACTAGCAGTCAATAGGGAC | |
| | GGTGGACTCTATAGAGCCCAGGTGC | |
| | CTCCCGAGGTAGTTGAACGGTCTGG | |
| | CATTGCAGAACGATTTTATGCAGAT | |
| | GATTGTGTTCTTACTACAACCGATT | |
| | ACATTTGCTCATCGATCCGATCTTC | |
| | TCGGCTTAATCCAGAGTTAGTTAGA | |
| | TGTCTCAGTGGGGCACTTGATTCAT | |
| | GCACATTTGAGAGGGAAAGTGCATT | |
| | ATTGTCAACCCCTTTCTTTGTATAC | |
| | AACAAGGCAGTTGTCGCAAATTGTA | |
| | AAGCAGCAACATGTAGATGTAATAA | |
| | ACCGCCGTCTATTATTGCCCAATAC | |
| | TCTGCATCAGCTCTGGTCACCATCA | |
| | CCACCGACACCTGTGCCGACCTCGA | |
| | AATTGAGGGTTATCGCTTCAACATA | |
| | CAGACTGAATCCAACTCATGGGTTG | |
| | CACCAAACTTCACTGTCTCGACTTC | |
| | ACAGATTGTATCAGTTGATCCCATA | |
| | GACATCTCTTCTGACATTGCCAAAA | |
| | TCAACAGTTCCATCGAGGCTGCAAG | |
| | AGAGCAGCTGGAACTAAGCAACCAG | |
| | ATCCTTTCCCGGATCAACCCACGAA | |
| | TCGTGAATGATGAATCACTGATAGC | |
| | TATTATCGTGACAATTGTTGTGCTT | |
| | AGTCCCCTCGTAATCGGTCTGATTG | |
| | TTGTTCTCGGTGTGATGTATAAGAA | |
| | TCTTAGGAAAGTCCAACGAGCTCAA | |
| | GCTGCCATGATGATGCAGCAAATGA | |
| | GCTCATCACAGCCTGTGACCACTAA | |
| | ATTAGGGACGCCTTTCTAGGAGAAC | |
| | AACCATATCACTCCACTCAATGATG | |
| | AGCAAGACGTACCAATCATCAATGA | |
| | TTGTGTCACAAGGCCGGTTGGGAAT | |
| | GCATCGAATCTCTCCCCTTTCTTTT | |
| | TAATTAAAAACATTTGAAGTGAAGA | |
| | TGAGAGGGGGGAAGTGTATGGTAGG | |
| | GTGGGGAAGGCAGCCAATTCCTGCC | |
| | CATTAGGCCGACCGTATCAAAAGGA | |
| | TTCAATAGAAGTCTAGGTACAGGGT | |
| | AACATGGAGGGCAGCCGCGATAATC | |
| | TTACAGTGGATGATGAATTAAAGAC | |
| | AACATGGAGGTTAGCTTATAGAGTT | |
| | GTGTCTCTTCTATTGATGGTGAGCG | |
| | CTTTGATAATCTCTATAGTAATCCT | |
| | GACGAGAGATAACAGCCAAAGCATA | |
| | ATCACGGCGATCAACCAGTCATCTG | |
| | ACGCAGACTCTAAGTGGCAAACGGG | |
| | AATAGAAGGGAAAATCACCTCCATT | |
| | ATGGCTGATACGCTCGATACCAGGA | |
| | ATGCAGTTCTTCTCCACATTCCACT | |
| | CCAGCTCAACACTCTTGAGGCGAAC | |
| | CTATTGTCTGCCCTTGGGGGCAACA | |
| | CAGGAATTGGCCCCGGAGATCTAGA | |
| | GCACTGCCGTTACCCTGTTCATGAC | |
| | ACCGCTTACCTGCATGGAGTTAATC | |
| | GATTACTCATCAATCAGACAGCTGA | |
| | TTATACAGCAGAAGGCCCCCTAGAT | |
| | CATGTGAACTTCATTCCAGCCCCGG | |
| | TTACGACTACTGGATGCACAAGGAT | |
| | ACCATCCTTTTCCGTGTCATCGTCC | |
| | ATTTGGTGCTATACACATAACGTGA | |
| | TTGAAACCGGTTGCAATGACCACTC | |
| | AGGTAGTAATCAATATATCAGCATG | |
| | GGAGTCATTAAGAGAGCGGGCAACG | |
| | GCCTACCTTACTTCTCAACAGTTGT | |
| | AAGTAAGTATCTGACTGATGGGTTG | |
| | AATAGGAAAAGCTGTTCTGTGGCTG | |
| | CCGGATCTGGGCATTGCTACCTCCT | |
| | TTGCAGCTTAGTGTCGGAGCCCGAA | |
| | CCTGATGACTATGTGTCACCTGATC | |
| | CTACACCGATGAGGTTAGGGGTGCT | |
| | AACGTGGGATGGATCTTACACTGAA | |
| | CAGGTGGTACCCGAAAGAATATTCA | |
| | GGAACATATGGAGTGCAAACTACCC | |
| | AGGAGTAGGGTCAGGTGCTATAGTA | |
| | GGAAATAAGGTGTTATTCCCATTTT | |
| | ACGGCGGAGTGAGGAATGGATCGAC | |
| | CCCGGAGGTGATGAATAGGGGAAGG | |
| | TACTACTACATCCAGGATCCAAATG | |
| | ACTATTGCCCTGACCCGCTGCAAGA | |
| | TCAGATCTTAAGGGCGGAACAATCG | |
| | TATTACCCAACTCGATTCGGTAGGA | |
| | GGATGATAATGCAGGGGGTCCTAGC | |
| | ATGTCCAGTATCCAACAATTCAACA | |
| | ATAGCAAGCCAATGTCAATCTTACT | |
| | ATTTTAATAACTCATTAGGGTTCAT | |
| | TGGAGCAGAATCTAGAATCTATTAC | |
| | CTCAATAGTAACATTTACCTTTATC | |
| | AGAGGAGCTCGAGCTGGTGGCCTCA | |
| | CCCCCAGATTTACCTGCTTGATTCT | |
| | AGGATTGCAAGTCCGGGTACTCAGA | |
| | ACATTGACTCAGGTGTCAATCTCAA | |
| | GATGTTAAACGTCACTGTGATTACA | |
| | CGACCATCATCTGGTTTTTGTAATA | |
| | GTCAGTCACGATGCCCTAATGACTG | |
| | CTTATTCGGGGTCTACTCGGATATC | |
| | TGGCCTCTTAGCCTTACCTCGGATA | |
| | GCATATTCGCGTTCACTATGTATTT | |
| | ACAGGGGAAGACAACACGTATTGAC | |
| | CCGGCTTGGGCGCTATTCTCCAATC | |
| | ATGCGATTGGGCATGAGGCTCGTCT | |
| | GTTTAATAAGGAGGTTAGTGCTGCT | |
| | TATTCTACCACCACTTGTTTTTTGG | |
| | ACACCATCCAAAACCAGGTGTATTG | |
| | CCTGAGTATACTTGAGGTCAGGAGT | |
| | GAGCTCTTGGGAGCATTCAAAATAG | |
| | TACCATTCCTCTATCGTGTCTTGTA | |
| | GGCATCCATTCGGCCAAAAAACTTG | |
| | AGTGACTATGAGGTTAACACTTGAT | |
| | CCCCCTTAAAGACACCTATCTAAAT | |
| | TACTGTCCTAGACCCATGATTAGGT | |
| | ACCTTTTAAATCAATCATTTGGTTT | |
| | TTAATTAAAAATGAAAAAATGGGCC | |
| | TAGTTTCAAGAGAGGGCTGGAACCC | |
| | ACTAGGGTGGGGAAGGATTGCTTTG | |
| | CTCCTTGACTCACACCCACGTATAC | |
| | TCGATCTCACTTCTGTAAAGAAGGG | |
| | ATCCTTCTCAAACTCGCCCCACAAT | |
| | GTCCAATCAGGCAGCTGAGATTATA | |
| | CTACCCACCTTCCATCTAGAATCAC | |
| | CCTTAATCGAGAATAAGTGCTTTTA | |
| | TTATATGCAATTACTTGGTCTCGTG | |
| | TTGCCACATGATCATTGGAGATGGA | |
| | GGGCATTCGTTAACTTTACAGTGGA | |
| | TCAGGTGCACCTTAAAAATCGTAAT | |
| | CCCCGCTTAATGGCCCATATCGACC | |
| | ACACTAAAGATAGATTAAGGACTCA | |
| | TGGTGTCTTAGGTTTCCACCAGACT | |
| | CAGACAAGTTTGAGCCGTTATCGTG | |
| | TTTTGCTCCATCCTGAAACCTTACC | |
| | TTGGCTATCAGCCATGGGAGGATGC | |
| | ATCAATCAGGTTCCTAAAGCATGGC | |
| | GGAATACTCTGAAATCGATCGAGCA | |
| | TAGTGTAAAGCAGGAGGCACCTCAA | |
| | CTAAAGCTACTCATGGAGAGAACCT | |
| | CATTAAAATTAACTGGAGTACCTTA | |
| | CTTGTTCTCTAATTGCAATCCCGGG | |
| | AAAACCACAGCAGGAACTATGCCTG | |
| | TCCTAAGTGAGATGGCATCGGAACT | |
| | CTTGTCAAATCCTATCTCCCAATTC | |
| | CAATCAACATGGGGGTGTGCTGCTT | |
| | CGGGGTGGCACCATGTAGTCAGTAT | |
| | CATGAGGCTCCAACAATATCAAAGA | |
| | AGGACAGGTAAGGAAGAGAAAGCAA | |
| | TCACCGAAGTTCAGTATGGCACAGA | |
| | CACTTGTCTCATTAACGCAGACTAT | |
| | ACCGTTGTTTTTTCCACACAGAACC | |
| | GTGTTATAACGGTCTTGCCCTTCGA | |
| | TGTTGTCCTCATGATGCAAGACCTA | |
| | CTCGAATCCCGACGGAATGTTCTGT | |
| | TCTGTGCCCGCTTTATGTATCCCAG | |
| | AAGCCAACTTCATGAGAGGATAAGT | |
| | GCAATATTAGCCCTTGGAGACCAAC | |
| | TGGGGAGAAAAGCACCCCAAGTCCT | |
| | GTATGATTTCGTGGCGACCCTCGAG | |
| | TCATTTGCATACGCAGCTGTTCAAC | |
| | TTCATGACAACAATCCTACCTACGG | |
| | TGGGGCCTTCTTTGAATTCAATATC | |
| | CAAGAGTTAGAATCTATTCTGTCCC | |
| | CTGCACTTAGTAAGGATCAGGTCAA | |
| | CTTCTACATAGGTCAAGTTGTCTCA | |
| | GCGTACAGTAACCTTCCTCCATCTG | |
| | AATCGGCAGAATTGTTGTGCCTGCT | |
| | ACGCCTGTGGGGTCATCCCTTGCTA | |
| | AACAGCCTTGATGCAGCAAAGAAAG | |
| | TCAGGGAGTCTATGTGTGCCGGGAA | |
| | GGTTCTCGATTACAACGCCATTCGA | |
| | CTCGTCTTGTCTTTTTACCATACAT | |
| | TGTTAATCAATGGGTACCGAAAGAA | |
| | ACACAAGGGTCGCTGGCCAAATGTG | |
| | AATCAACATTCACTCCTCAACCCGA | |
| | TAGTGAGGCAGCTCTATTTTGATCA | |
| | GGAAGAGATCCCACACTCTGTTGCC | |
| | CTTGAGCACTATTTGGATGTCTCAA | |
| | TGATAGAATTTGAAAAAACTTTTGA | |
| | AGTGGAACTATCTGACAGCCTAAGC | |
| | ATCTTCCTGAAGGATAAGTCGATAG | |
| | CTTTGGATAAGCAAGAATGGTACAG | |
| | TGGTTTTGTCTCAGAAGTGACTCCG | |
| | AAGCACCTACGAATGTCTCGTCATG | |
| | ATCGCAAGTCTACCAATAGGCTCCT | |
| | GTTAGCTTTCATTAACTCCCCTGAA | |
| | TTCGACGTTAAGGAGGAGCTTAAGT | |
| | ACTTGACTACGGGTGAGTACGCCAC | |
| | TGACCCAAATTTCAATGTCTCATAC | |
| | TCACTTAAAGAGAAGGAAGTAAAAA | |
| | AAGAAGGGCGCATATTCGCAAAAAT | |
| | GTCACAAAAGATGAGAGCATGCCAG | |
| | GTTATTTGTGAAGAATTGCTAGCAC | |
| | ATCATGTGGCTCCTTTGTTTAAAGA | |
| | GAATGGTGTTACTCAATCAGAGCTA | |
| | TCCCTGACAAAAAATTTGTTGGCTA | |
| | TTAGCCAACTGAGTTACAACTCGAT | |
| | GGCTGCTAAGGTGCGATTGCTGAGG | |
| | CCAGGGGACAAGTTCACTGCTGCAC | |
| | ACTATATGACCACAGACCTAAAGAA | |
| | GTACTGTCTCAATTGGCGGCACCAG | |
| | TCAGTCAAACTGTTCGCCAGAAGCC | |
| | TGGATCGACTGTTTGGATTAGACCA | |
| | TGCGTTTTCTTGGATACATGTCCGT | |
| | CTCACCAACAGCACTATGTACGTTG | |
| | CTGACCCCTTCAATCCACCAGACTC | |
| | AGAGGCATGCACAGATTTAGACGAC | |
| | AATAAGAACACCGGGATTTTTATTA | |
| | TAAGTGCAAGAGGTGGTATAGAAGG | |
| | CCTCCAACAAAAATTATGGACTGGC | |
| | ATATCGATTGCAATTGCCCAAGCGG | |
| | CAGCGGCCCTCGAAGGCTTACGAAT | |
| | TGCTGCTACTCTGCAGGGGGATAAC | |
| | CAAGTTTTGGCGATTACGAAGGAAT | |
| | TCATGACCCCAGTCCCAGAGGATGT | |
| | AATCCATGAGCAGCTATCTGAGGCG | |
| | ATGTCTCGATACAAAAGGACTTTCA | |
| | CATACCTCAATTATTTAATGGGGCA | |
| | TCAGTTGAAGGATAAAGAAACCATC | |
| | CAATCCAGTGACTTCTTTGTTTATT | |
| | CCAAAAGAATCTTCTTCAATGGATC | |
| | GATCTTAAGTCAATGCCTCAAAAAC | |
| | TTCAGTAAACTCACTACTAATGCCA | |
| | CTACCCTTGCTGAGAATACTGTGGC | |
| | CGGCTGCAGTGACATCTCTTCATGC | |
| | ATTGCCCGTTGTGTGGAAAACGGGT | |
| | TGCCTAAGGATGCCGCATATATCCA | |
| | GAATATAATCATGACTCGGCTTCAA | |
| | CTATTGCTAGATCATTACTATTCAA | |
| | TGCATGGCGGCATAAATTCAGAATT | |
| | AGAGCAGCCAACTTTAAGTATCTCT | |
| | GTTCGAAACGCAACCTACTTACCAT | |
| | CTCAACTAGGCGGTTACAATCATTT | |
| | GAATATGACCCGACTATTCTGCCGC | |
| | AATATCGGCGACCCGCTTACCAGTT | |
| | CTTGGGCGGAGTCAAAAAGACTAAT | |
| | GGATGTTGGTCTCCTCAGTCGTAAG | |
| | TTCTTAGAGGGGATATTATGGAGAC | |
| | CCCCGGGAAGTGGGACGTTTTCAAC | |
| | ACTCATGCTTGACCCGTTCGCACTT | |
| | AACATTGATTACCTGAGGCCGCCAG | |
| | AGACAATTATCCGAAAACACACCCA | |
| | AAAAGTCTTGTTGCAAGATTGCCCA | |
| | AATCCCCTATTAGCAGGTGTCGTTG | |
| | ACCCGAACTACAACCAAGAATTAGA | |
| | GCTGTTAGCTCAGTTCTTGCTTGAT | |
| | CGGGAAACCGTTATTCCCAGGGCTG | |
| | CCCATGCCATCTTCGAGTTATCTGT | |
| | CTTGGGAAGGAAAAAACATATACAA | |
| | GGATTGGTAGATACTACAAAGACAA | |
| | TTATTCAGTGCTCATTGGAAAGACA | |
| | GCCATTGTCTTGGAGGAAAGTTGAG | |
| | AACATTGTTACCTACAACGCGCAGT | |
| | ATTTCCTCGGGGCCACCCAACAGGC | |
| | TGATACTAATGTCTCAGAAGGGCAG | |
| | TGGGTGATGCCAGGTAACCTTAAGA | |
| | AGCTTGTGTCCCTCGACGATTGCTC | |
| | GGTCACGCTGTCTACTGTATCACGG | |
| | CGCATATCATGGGCCAATCTACTGA | |
| | ACTGGAGAGCTATAGATGGTCTGGA | |
| | AACCCCGGATGTGATAGAGAGTATT | |
| | GATGGTCGCCTTGTACAATCATCCA | |
| | ATCAATGTGGCCTATGTAATCAAGG | |
| | GTTGGGATCCTACTCCTGGTTTTTC | |
| | TTGCCCTCTGGGTGTGTGTTCGACC | |
| | GTCCACAAGATTCTCGGGTAGTTCC | |
| | AAAGATGCCATACGTGGGGTCCAAA | |
| | ACAGATGAGAGACAGACTGCATCAG | |
| | TGCAAGCTATACAAGGATCCACTTG | |
| | TCACCTCAGGGCAGCATTGAGGCTT | |
| | GTATCACTCTACCTATGGGCCTATG | |
| | GAGATTCTGACATATCATGGCTAGA | |
| | AGCTGCAACGCTGGCTCAAACACGG | |
| | TGCAATGTCTCTCTCGATGATTTGC | |
| | GAATCTTGAGCCCTCTTCCTTCTTC | |
| | GGCGAATTTACACCACAGATTAAAT | |
| | GACGGGGTAACACAGGTTAAATTCA | |
| | TGCCCGCCACATCTAGCCGAGTGTC | |
| | AAAGTTCGTCCAAATTTGCAATGAC | |
| | AACCAGAATCTTATCCGTGATGATG | |
| | GGAGTGTTGATTCCAATATGATTTA | |
| | TCAACAGGTTATGATATTAGGGCTT | |
| | GGAGAGATTGAATGCTTGTTAGCTG | |
| | ACCCAATTGATACAAACCCAGAACA | |
| | ATTGATTCTTCATCTACACTCTGAT | |
| | AATTCTTGCTGTCTCCGGGAGATGC | |
| | CAACGACCGGCTTTGTACCTGCTCT | |
| | AGGACTAACCCCATGTTTAACTGTC | |
| | CCAAAGCATAATCCTTACATTTATG | |
| | ACGATAGCCCAATACCCGGTGATTT | |
| | GGATCAGAGGCTCATTCAGACCAAA | |
| | TTTTTCATGGGGTCTGACAATTTGG | |
| | ATAATCTTGATATCTACCAGCAGCG | |
| | AGCTTTACTGAGTAGGTGTGTAGCT | |
| | TATGATGTCATCCAATCGATCTTTG | |
| | CCTGTGATGCACCAGTCTCTCAGAA | |
| | GAATGACGCAATCCTTCACACTGAT | |
| | TACCATGAGAATTGGATCTCAGAGT | |
| | TCCGATGGGGTGACCCTCGTATTAT | |
| | CCAAGTAACGGCAGGCTATGAGTTA | |
| | ATTCTGTTCCTTGCATACCAGCTTT | |
| | ATTATCTCAGAGTGAGGGGTGACCG | |
| | TGCAATCCTGTGCTATATCGACAGG | |
| | ATACTCAATAGGATGGTATCTTCCA | |
| | ATCTAGGTAGTCTCATCCAGACACT | |
| | CTCTCATCCAGAGATTAGGAGGAGA | |
| | TTCTCGTTGAGTGATCAAGGGTTTC | |
| | TTGTTGAAAGAGAACTAGAGCCAGG | |
| | TAAGCCCTTGGTTAAACAAGCGGTT | |
| | ATGTTCTTAAGGGACTCGGTCCGCT | |
| | GCGCTTTAGCAACTATCAAGGCAGG | |
| | AATTGAGCCTGAAATCTCCCGAGGT | |
| | GGTTGTACTCAGGATGAGCTGAGCT | |
| | TTACTCTTAAGCACTTACTATGTCG | |
| | GCGTCTCTGTGTAATCGCTCTCATG | |
| | CATTCAGAAGCAAAGAACTTGGTTA | |
| | AAGTTAGAAACCTTCCTGTAGAAGA | |
| | GAAAACCGCCTTATTGTACCAGATG | |
| | TTGGTCACTGAGGCCAATGCTAGGA | |
| | AATCAGGGTCTGCCAGCATTATCAT | |
| | AAACCTAGTCTCGGCACCCCAGTGG | |
| | GACATTCATACACCAGCATTGTATT | |
| | TTGTGTCAAAGAAAATGCTAGGGAT | |
| | GCTTAAGAGGTCAACCACACCCTTG | |
| | GATATAAGTGACCTCTCTGAGAACC | |
| | AGAACCCCGCACCTGCAGAGCTTAG | |
| | TGATGCTCCTGGTCACATGGCAGAA | |
| | GAATTCCCCTGTTTGTTTAGTAGTT | |
| | ATAACGCTACATATGAAGACACAAT | |
| | CACTTACAATCCAATGACTGAAAAA | |
| | CTCGCCTTGCATTTGGACAACAGTT | |
| | CCACCCCATCCAGAGCACTTGGTCG | |
| | TCACTACATCCTGCGGCCTCTTGGG | |
| | CTTTACTCATCCGCATGGTACCGGT | |
| | CTGCGGCACTACTAGCGTCAGGGGC | |
| | CCTAAATGGGTTGCCTGAGGGGTCG | |
| | AGCCTGTATTTAGGAGAAGGGTACG | |
| | GGACCACCATGACTCTGCTTGAGCC | |
| | CGTTGTCAAGTCTTCAACTGTTTAC | |
| | TACCATACATTGTTTGACCCAACCC | |
| | GGAACCCTTCACAGCGGAACTATAA | |
| | ACCAGAACCACGGGTATTCACGGAT | |
| | TCTATTTGGTACAAGGATGATTTCA | |
| | CACGGCCACCCGGTGGTATTATCAA | |
| | CCTGTGGGGTGAAGATATACGTCAG | |
| | AGTGATATCACACAGAAAGACACGG | |
| | TCAACTTCATACTATCTCAGATCCC | |
| | GCCAAAATCACTTAAGTTGATACAC | |
| | GTTGATATTGAGTTCTCACCAGACT | |
| | CCGATGTACGGACACTACTATCCGG | |
| | CTATTCTCATTGTGCACTATTGGCC | |
| | TACTGGCTATTGCAACCTGGAGGGC | |
| | GATTCGCAGTTAGGGTTTTCTTAAG | |
| | TGACCATATCATAGTTAACTTGGTC | |
| | ACTGCGATCCTGTCTGCTTTTGACT | |
| | CCAATTTGGTGTGCATTGCGTCAGG | |
| | ATTGACACACAAGGATGATGGGGCA | |
| | GGTTATATTTGCGCGAAAAAGCTTG | |
| | CAAATGTTGAGGCTTCAAGAATTGA | |
| | GTACTACTTGAGGATGGTCCATGGT | |
| | TGTGTTGACTCATTAAAGATCCCTC | |
| | ATCAATTAGGAATCATTAAATGGGC | |
| | CGAGGGTGAGGTGTCCCAGCTTACC | |
| | AGAAAGGCGGATGATGAAATAAATT | |
| | GGCGGTTAGGTGATCCAGTTACCAG | |
| | ATCATTTGATCCAGTTTCTGAGCTA | |
| | ATAATTGCACGAACAGGGGGGTCTG | |
| | TATTAATGGAATACGGGGCTTTTAC | |
| | TAACCTCAGGTGTGCGAACTTGGTA | |
| | GATACATACAAACTTCTGGCTTCAA | |
| | TTGTAGAGACCACCCTAATGGAAAT | |
| | AAGGGTTGAGCAAGATCAGTTGGAA | |
| | GATAGTTCGAGGAGACAAATCCAAG | |
| | TAATCCCCGCTTTCAACACAAGATC | |
| | TGGGGGAAGGATCCGTACACTGATT | |
| | GAGTGTGCTCAGCTGCAGATTATAG | |
| | ATGTTATTTGTGTAAACATAGATCA | |
| | CCTCTTTCCTAAACACCGACATGTT | |
| | CTTGTCACACAACTTACCTACCAGT | |
| | CGGTGTGCCTTGGGGATTTGATTGA | |
| | AGGTCCCCAAATTAAGACGTATCTA | |
| | AGGGCCAGAAAGTGGATCCAACGTC | |
| | GGGGACTCAATGAGACAGTTAACCA | |
| | TATCATCACTGGACAAGTGTCACGG | |
| | AATAAAGCAAGGGATTTTTTTAAGA | |
| | GGCGCCTGAAGTTGGTTGGCTTTTC | |
| | ACTCTGCGGAGGTTGGAGCTACCTC | |
| | TCACTTTAGCTGTTCAGGTTGCTGA | |
| | TCATCATGAACAATCGGAGTCGGAA | |
| | TCGTAAACAGAAAGTCACAAAATTG | |
| | TGGATAAACAATGATTGCATTAGTA | |
| | TTTAATAAAAAATATGTCTTTTATT | |
| | TCGT | |
| Avian | ACGAAAAAGAAGAATAAAAGGCAGA | SEQ ID |
| paramyxovir | AGCCTTTTAAAAGGAACCCTGGGCT | NO: 8 |
| us 4 strain | GTCGTAGGTGTGGGAAGGTTGTATT | |
| APMV- | CCGAGTGCGCCTCCGAGGCATCTAC | |
| 4/duck/ | TCTACACCTATCACAATGGCTGGTG | |
| Delaware/ | TCTTTTCCCAGTATGAGAGGTTTGT | |
| 549227/ | GGACAATCAATCTCAGGTGTCAAGG | |
| 2010, | AAGGATCATCGGTCCTTAGCAGGAG | |
| complete | GGTGCCTTAAAGTGAACATCCCTAT | |
| genome | GCTTGTCACTGCATCCGAAGACCCC | |
| Genbank: | ACCACGCGTTGGCAACTAGCATGCT | |
| JX987283.1 | TATCTCTGAGGCTCTTGATTTCCAA | |
| | TTCATCAACCAGTGCTATCCGCCAG | |
| | GGAGCAATACTGACCCTCATGTCAT | |
| | TGCCATCGCAAAACATGAGAGCAAC | |
| | AGCAGCTATTGCTGGGTCCACGAAT | |
| | GCGGCTGTTATCAACACTATGGAAG | |
| | TCTTAAGTGTCAATGACTGGACCCC | |
| | ATCTTTTGACCCAAGAAGTGGTCTA | |
| | TCTGAGGAGGACGCTCAGGTGTTCA | |
| | GAGACATGGCAAGAGATCTGCCTCC | |
| | TCAGTTCACTTCTGGATCACCCTTT | |
| | ACATCAGCATTGGCGGAGGGGTTTA | |
| | CTCCCGAGGACACTCATGACCTGAT | |
| | GGAGGCACTGACTAGTGTACTGATA | |
| | CAGATCTGGATTCTGGTGGCCAAGG | |
| | CCATGACCAATATTGATGGATCTGG | |
| | GGAGGCTAACGAAAGACGCCTTGCA | |
| | AAATACATCCAAAAGGGACAGCTCA | |
| | ATCGTCAGTTTGCAATTGGCAATCC | |
| | TGCCCGTCTGATAATCCAACAGACA | |
| | ATCAAAAGCTCATTAACTGTCCGCA | |
| | GGTTCTTGGTCTCTGAGCTCCGCGC | |
| | ATCACGTGGTGCAGTAAAGGAGGGT | |
| | TCCCCTTACTATGCAGCCGTTGGGG | |
| | ATATCCACGCTTACATCTTCAATGC | |
| | AGGATTGACACCATTCTTGACCACC | |
| | CTGAGATATGGCATTGGCACCAAGT | |
| | ACGCCGCTGTCGCACTCAGTGTGTT | |
| | TGCTGCAGACATTGCAAAATTGAAG | |
| | AGTCTACTCACCCTGTATCAAGACA | |
| | AAGGTGTAGAAGCTGGATACATGGC | |
| | ACTCCTTGAAGATCCAGATTCCATG | |
| | CACTTTGCACCTGGAAACTTCCCAC | |
| | ACATGTATTCCTATGCGATGGGAGT | |
| | GGCCTCCTATCACGACCCTAGCATG | |
| | CGCCAATACCAGTATGCCAGGAGGT | |
| | TTCTCAGTCGTCCCTTCTACCTGCT | |
| | AGGAAGAGACATGGCTGCTAAGAAC | |
| | ACAGGAACTCTGGATGAGCAGCTGG | |
| | CGAAAGAACTGCAAGTGTCAGAGAG | |
| | GGACCGCGCTGCACTGTCTGCCGCG | |
| | ATTCAATCAGCAATGGAGGGGGGAG | |
| | AGTCAGATGACTTCCCATTGTCAGG | |
| | ATCCATGCCGGCCCTCTCTGAGAGC | |
| | ACACAACCGGTCACCCCCAGGACTC | |
| | AACAGTCCCAGCTCTCTCCTCCTCA | |
| | ATCATCAAACATGTCCCAATCGGCG | |
| | CCTAGGACCCCGGACTATCAACCCG | |
| | ACTTTGAGCTGTAGACTATATCCAC | |
| | ACACCGACAATAGCTCCAGAAGACC | |
| | CCCTTCCCCCCCATACACCCCACCC | |
| | GGTCATCCACAAAGACCCAGTCCAA | |
| | CATCCCAGCACTATTCCCTTTTAAT | |
| | TAAAAACTGGCCGACAGGGTGGGGA | |
| | AGGAGGACTGTTAGCTGCCACCAAC | |
| | GGTGTGCAGCAATGGATTTTACAGA | |
| | CATTGACGCTGTCAACTCACTGATT | |
| | GAGTCATCATCGGCAATTATAGACT | |
| | CCATACAGCATGGAGGGCTGCAACC | |
| | AGCAGGCACTGTTGGCTTATCTCAA | |
| | ATTCCAAAAGGGATAACCAGTGCAC | |
| | TGAATAAAGCCTGGGAAGCTGAGGC | |
| | GGCAACTGCCGGCAGTGGAGACACC | |
| | CAACACAAACCCGATGACCCAGAGG | |
| | ACCACCAGGCTAGGGACACGGAGTC | |
| | CCTGGAAGACACAGGCAACGACCCG | |
| | GCCACACAGGGGACTAACATTGTTG | |
| | AGACACCCCACCCAGAAGTACTGTC | |
| | AGCAGCCAAAGCTAGACTCAAGAGA | |
| | CCCAAAGCAGGGAAAGACACCCATG | |
| | GCAATCCCCCCACTCAACCCGATCA | |
| | CTTTTTAAAGGGGGGCCTCCCGAGT | |
| | CCACAACCGACAGCACCGCGGATGC | |
| | AAAGTCCACCCAACCATGGAAGCTC | |
| | CAGCACCGCCGATCCCCGCCAATCA | |
| | CAAACTCAGGATCATTCCCCCACCG | |
| | GAGAGAAATGGCAATTGTCACCGAC | |
| | AAAGCAACCGGAGACATCGAACTGG | |
| | TGGAGTGGTGCAACCCAGGGTGTAC | |
| | AGCAGTCCGAATTGAACCAGCCAGA | |
| | CTTGACTGTGTATGCGGACACTGCC | |
| | CCACCATCTGCAGTCTCTGCATGTA | |
| | TGACGACTGATCAGGTACAGTTGTT | |
| | GATGAAGGAGGTTGCTGACATAAAA | |
| | TCACTCCTCCAGGCACTAGTAAGGA | |
| | ATCTAGCTGTCTTGCCCCAACTAAG | |
| | GAATGAGGTTGCAGCAATCAGAACA | |
| | TCACAGGCCATGATAGAGGGGACAC | |
| | TCAATTCAATTAAGATTCTTGATCC | |
| | TGGAAATTATCAGGAATCATCACTA | |
| | AACAGTTGGTTCAAACCTCGCCAGG | |
| | AACACACTGTTATTGTGTCAGGACC | |
| | AGGGAATCCACTGGCCATGCCGACT | |
| | CCAGTTCAGGACAGTACCATATTCT | |
| | TAGATGAGCTAGCAAGACCTCATCC | |
| | TAATTTGGTCAATCCGTCTCCGCCC | |
| | GTCACCAGCACCAATGTTGACCTTG | |
| | GCCCACAGAAGCAGGCTGCAATAGC | |
| | CTACGTTTCCGCCAAGTGCAAGGAC | |
| | CCAGGGAAACGGGACCAGCTTTCAA | |
| | GGCTTATTGAACGGGCGGCTACCTT | |
| | GAGTGAGATCAACAAGGTTAAAAGA | |
| | CAGGCTCTCGGGCTCTAAATTAATC | |
| | AACCACCCGTTGCAACGATCGAGAC | |
| | AACAATAAAAATCCCCCTGAATCAC | |
| | ATGACCAAATCTGCATACCACTCAC | |
| | ATCATCCGCCTATACCCCTCACCAT | |
| | AAATACCACCTTAGCCGATTTATTT | |
| | AAAAGAAATCATTCATCACAACCTG | |
| | GTAATCATAAACTAGGGTGGGGAAG | |
| | GTCTCTTGTCTGCAGGAAGGCTCCT | |
| | CTGTCTCCAGGCACGCACCCGTCAA | |
| | CCCACCAATAACACAATGGCGGACA | |
| | TGGACACGATATACATCAACTTGAT | |
| | GGCAGATGATCCAACCCATCAAAAA | |
| | GAATTGCTGTCATTCCCTCTGATTC | |
| | CAGTGACTGGACCTGATGGGAAGAA | |
| | AGTGCTCCAACACCAGATCCGGACC | |
| | CAATCCTTGCTCACCTCAGACAAAC | |
| | AAACGGAGAGGTTCATCTTTCTCAA | |
| | CACTTACGGGTTCATCTATGACACA | |
| | ACCCCGGACAAGACAACTTTTTCCA | |
| | CCCCTGAGCATATCAATCAGCCTAA | |
| | GAGGACAATGGTGAGTGCTGCGATG | |
| | ATGACTATTGGTCTGGTTCCTGCTA | |
| | CAATACCCCTGAATGAATTGACGGC | |
| | CACTGTGTTTAACCTTAAAGTAAGA | |
| | GTGAGGAAAAGTGCGAGGTATCGAG | |
| | AAGTGGTTTGGTACCAGTGCAACCC | |
| | CGTACCAGCTCTGCTCGCAGCCACC | |
| | AGATTTGGCCGCCAAGGGGGTCTTG | |
| | AGTCGAGCACCGGAGTCAGTGTAAA | |
| | GGCACCTGAGAAGATTGATTGTGAG | |
| | AAAGATTATACTTACTACCCTTATT | |
| | TCCTATCTGTGTGCTACATCGCCAC | |
| | TTCCAACCTCTTTAAGGTACCGAAG | |
| | ATGGTTGCCAATGCAACCAACAGTC | |
| | AATTGTATCACCTAACCATGCAGGT | |
| | CACATTTGCATTTCCGAAAAACATT | |
| | CCCCCAGCCAATCAGAAACTCCTGA | |
| | CACAGGTAGATGAAGGATTTGAGGG | |
| | TACCGTGGATTGCCATTTTGGGAAC | |
| | ATGCTAAAAAAGGATAGGAAAGGGA | |
| | ACATGAGGACTTTGTCTCAAGCAGC | |
| | AGATAAGGTCAGAAGAATGAATATC | |
| | CTTGTGGGAATATTTGACTTGCACG | |
| | GACCTACACTATTCCTGGAATATAC | |
| | TGGGAAATTGACAAAAGCCCTGTTG | |
| | GGGTTCATGTCCACCAGCCGAACAG | |
| | CAATCATCCCCATATCACAACTCAA | |
| | TCCTATGCTGAGTCAACTCATGTGG | |
| | AGCAGTGACGCCCAGATAGTAAAGT | |
| | TACGGGTGGTCATCACTACATCTAA | |
| | ACGTGGCCCGTGTGGGGGCGAGCAG | |
| | GAATATGTGCTGGATCCTAAATTCA | |
| | CAGTTAAGAAAGAAAAGGCTCGACT | |
| | CAATCCATTCAAGAAGGCAGCCTAA | |
| | TAATTAAACCTACAAGATCCCAAGA | |
| | ATTAAACAGCTCTATACAATTCATA | |
| | GGTTGATAGAAATGCCACTACACAG | |
| | CTAATGATTTTCCAGAAAATCACTT | |
| | AGAAAACCAAATCCTTATTAGGGTG | |
| | GGGAAGTAGTTGATTGGGTGTCTAA | |
| | ACAAAAGTGCTTCTTTGCAACTCCC | |
| | CACCCCGAAGCAATCACAATGAGAC | |
| | CATTAAACACGCTTTTGACCGTGAT | |
| | TCTTATCATACTCATCAGCTATTTG | |
| | GTGATTGTTCATTCTAGTGATGCGG | |
| | TTGAGAGGCCAAGGACTGAGGGAAT | |
| | TAGGGGCGACCTCATTCCAGGTGCG | |
| | GGTATCTTCGTGACTCAAGTCCGAC | |
| | AACTGCAAATCTATCAGCAGTCAGG | |
| | GTACCACGACCTTGTCATAAGATTA | |
| | TTACCCCTTTTACCAACGGAACTCA | |
| | ATGATTGCCAAAAAGAAGTAGTCAC | |
| | AGAATACAATAATACAGTATCACAA | |
| | TTGTTGCAGCCTATCAAAACCAACT | |
| | TGGATACCCTATTAGCAGATGGTAA | |
| | TACGAGGGAAGCGGATATACAGCCG | |
| | CGGTTTATTGGAGCAATAATAGCCA | |
| | CAGGTGCCTTGGCGGTAGCAACAGT | |
| | GGCAGAAGTAACTGCAGCTCAGGCA | |
| | CTCTCCCAGTCCAAAACAAATGCTC | |
| | AAAATATTCTCAAGCTAAGAGATAG | |
| | TATCCAGGCCACCAACCAAGCGGTC | |
| | TTTGAAATTTCACAAGGGCTTGAGG | |
| | CAACTGCAACTGTGCTATCGAAACT | |
| | ACAGACAGAGCTCAATGAGAATATT | |
| | ATCCCAAGCCTGAACAATTTATCCT | |
| | GTGCTGCCATGGGGAATCGTCTTGG | |
| | TGTATCACTCTCACTCTATTTAACT | |
| | CTAATGACTACCCTCTTTGGGGACC | |
| | AAATTACGAACCCAGTGCTGACACC | |
| | AATTTCTTACAGCACACTATCGGCA | |
| | ATGGCAGGTGGTCATATTGGCCCAG | |
| | TGATGAGTAAAATATTAGCCGGATC | |
| | GGTCACGAGCCAGTTGGGGGCAGAA | |
| | CAATTGATTGCTAGTGGCTTAATAC | |
| | AATCACAGGTGGTAGGCTATGATTC | |
| | CCAGTATCAATTATTGGTAATCAGG | |
| | GTTAACCTTGTTCGGATTCAGGAAG | |
| | TCCAGAATACCAGGGTTGTATCATT | |
| | AAGAACGCTAGCTGTCAATAGAGAT | |
| | GGTGGACTTTATAGAGCCCAAGTTC | |
| | CACCTGAGGTAGTCGAACGATCCGG | |
| | CATTGCAGAGCGGTTTTACGCAGAT | |
| | GATTGTGTTCTCACCACGACCGACT | |
| | ATATTTGCTCATCAATCAGATCCTC | |
| | TCGGCTTAATCCAGAATTAGTCAAG | |
| | TGTCTCAGTGGGGCACTTGATTCAT | |
| | GTACATTCGAGAGGGAGAGTGCCCT | |
| | GTTATCAACTCCTTTCTTTGTGTAC | |
| | AATAAGGCTGTCGTAGCAAATTGCA | |
| | AAGCGGCAACATGCAGATGCAACAA | |
| | ACCACCGTCAATTATTGCTCAATAT | |
| | TCTGCATCAGCTCTAGTAACCATCA | |
| | CCACTGACACCTGTGCCGATCTCGA | |
| | AATTGAGGGTTACCGTTTCAACATA | |
| | CAGACTGAATCTAACTCGTGGGTTG | |
| | CACCTAACTTTACTGTCTCAACCTC | |
| | ACAGATAGTGTCAGTTGATCCAATA | |
| | GACATATCCTCTGACATCGCAAAAA | |
| | TCAACAATTCGATTGAGGCCGCACG | |
| | AGAGCAGCTAGAACTGAGCAACCAG | |
| | ATCCTATCCCGGATTAACCCCCGAA | |
| | TCGTGAATGACGAATCACTGATAGC | |
| | TATTATCGTGACAATTGTTGTGCTT | |
| | AGTCTCCTTGTAGTCGGTCTTATCA | |
| | TTGTTCTCGGCGTGATGTATAAAAA | |
| | TCTCAAGAAGGTCCAACGAGCTCAG | |
| | GCTGCTATGATGATGCAGCAAATGA | |
| | GTTCATCGCAGCCTGTAACCACAAA | |
| | ACTGGGGACACCCTTCTAGGTGAAT | |
| | AAATGCATCACCTCTTTCCTTGATG | |
| | AGCGAGATGTCTTAATCATTGATAA | |
| | TTATGCCGTAAGGCTGGTAGGGAAT | |
| | GTGCTGAATCTCTCCTCTTCCTTTT | |
| | TAATTAAAAACGGTTGAACTGAGGG | |
| | GGAGAATGTGCATGGTAGGGTGGGG | |
| | AAGGTGTCTGATTCCTACCTATCGG | |
| | GCCAACTGTACCAGTAGAAGCTAAC | |
| | AGGAATTCTAATGCAGAGTGACATG | |
| | GAGGGCAGTCGTGATAACCTCACAG | |
| | TGGATGATGAGTTAAAGACAACATG | |
| | GAGGTTAGCTTACAGAGTTGTATCT | |
| | CTCCTATTAATGGTGAGTGCTTTGA | |
| | TAATTTCTATAGTAATCTTGACGAG | |
| | GGATAACAGCCAAAGCATAATCACG | |
| | GCAATCAACCAGTCATATGATGCAG | |
| | ACTCAAAGTGGCAAACAGGGATAGA | |
| | GGGGAAAATCACCTCTATCATGACT | |
| | GATACGCTTGATACTAGGAATGCAG | |
| | CTCTCCTCCACATTCCACTCCAACT | |
| | TAATACACTTGAAGCAAACCTATTA | |
| | TCAGCCCTCGGTGGCAACACAGGAA | |
| | TCGGCCCCGGGGATCTAGAGCATTG | |
| | CCGTTATCCAGTTCATGATTCTGCT | |
| | TACCTGCATGGAGTCAACCGATTAC | |
| | TTATCAATCAAACGGCTGATTATAC | |
| | AGCAGAGGGTCCACTAGATCATGTG | |
| | AACTTCATACCGGCACCAGTTACGA | |
| | CCACTGGATGCACTAGGATACCATC | |
| | TTTTTCCGTGTCCTCATCCATTTGG | |
| | TGTTATACTCACAATGTGATTGAAA | |
| | CTGGTTTTAATGATCACTCAGGCAG | |
| | CAATCAGTATATTAGCATGGGGGTG | |
| | ATTAAGAGGGCTGGCAACGGCTTGC | |
| | CTTATTTCTCAACCGTTGTGAGTAA | |
| | GTATCTGACCGACGGATTGAATAGG | |
| | AAAAGTTGTTCTGTGGCTGCTGGGT | |
| | CTGGGCATTGCTATCTTCTCTGCAG | |
| | CCTAGTATCAGAGCCCGAGCCTGAC | |
| | GACTATGTATCACCAGACCCCACAC | |
| | CGATGAGGTTAGGGGTTCTGACATG | |
| | GGATGGGTCCTATACTGAACAGGTG | |
| | GTGCCTGAAAGGATATTCAAAAACA | |
| | TATGGAGTGCAAATTACCCTGGGGT | |
| | GGGATCAGGTGCTATTGTGGGAAAT | |
| | AAGGTGTTGTTCCCATTTTACGGAG | |
| | GAGTGAGGAATGGGTCGACACCTGA | |
| | GGTTATGAATAGGGGAAGGTATTAC | |
| | TACATTCAAGATCCTAATGATTATT | |
| | GTCCTGATCCACTGCAAGACCAAAT | |
| | CTTAAGGGCAGAACAATCATATTAT | |
| | CCTACACGGTTTGGTAGGAGGATGG | |
| | TGATGCAGGGTGTCTTAGCGTGCCC | |
| | AGTGTCCAACAACTCAACAATTGCC | |
| | AGCCAATGCCAGTCCTACTATTTCA | |
| | ACAACTCATTAGGGTTCATTGGGGC | |
| | GGAATCTAGGATTTATTACCTAAAT | |
| | GGGAACCTCTACCTTTACCAAAGAA | |
| | GCTCGAGCTGGTGGCCCCACCCCCA | |
| | GATTTATCTGCTTGACCCCAGAATT | |
| | GCAAGCCCGGGCACTCAGAACATCG | |
| | ACTCAGGCATTAATCTCAAGATGTT | |
| | GAATGTTACCGTTATTACACGACCG | |
| | TCATCTGGTTTTTGTAATAGTCAGT | |
| | CAAGATGCCCTAATGACTGCTTATT | |
| | CGGGGTCTATTCAGACGTCTGGCCT | |
| | CTTAGCCTAACCTCAGATAGTATAT | |
| | TCGCATTCACGATGTATTTACAAGG | |
| | GAAGACAACACGTATTGACCCGGCG | |
| | TGGGCACTGTTCTCCAATCACGCAA | |
| | TTGGGCATGAAGCTCGTCTATTCAA | |
| | CAAGGAGGTCAGTGCTGCTTACTCC | |
| | ACTACCACTTGCTTTTCGGACACCA | |
| | TCCAAAACCAGGTGTATTGCCTGAG | |
| | TATACTTGAAGTTAGAAGTGAGCTT | |
| | TTGGGGCCATTCAAGATAGTACCAT | |
| | TCCTCTACCGTGTCCTATAGGTGCC | |
| | TGCTCGATCGAGAACTCCAAATAAT | |
| | CGTGGAATTAGTACTTAATCTTCCC | |
| | TATGGATATCTGCCTTAATTACTGT | |
| | CCTAGGTCTCTGGATTAGCGCCCTT | |
| | TAAACCAGTTTTTTGATTTTTAATT | |
| | AAAAATAGAAGATTAGACCTGGACT | |
| | CGGGGAGGGAGAAGAACCTATTAGG | |
| | GTGGGGAAGGATTACTTTACTCCAT | |
| | GACTCACAATCGCACACACCTGACC | |
| | TCATTTCCACTGAGAAGGAACCCTC | |
| | CTCAAATTTGATTTGCAATGTCCAA | |
| | TCAAGCAGCTGAGATTATACTCCCT | |
| | ACCTTTCACCTAGAGTCACCCTTAA | |
| | TCGAGAACAAATGCTTCTACTATAT | |
| | GCAATTACTTGGTCTTATGTTGCCG | |
| | CATGATCATTGGAGATGGAGGGCAT | |
| | TTGTCAACTTTACAGTGGATCAAGC | |
| | ACACCTTAGAAACCGTAATCCTCGC | |
| | TTGATGGCCCACATCGACCACACTA | |
| | AGGATAAACTAAGGGCTCATGGTGT | |
| | CTTAGGTTTCCATCAGACCCAAACA | |
| | GGTGAGAGCCGTTTCCGTGTCTTGC | |
| | TTCACCCGGAAACCTTACCATGGCT | |
| | ATCAGCAATGGGAGGATGCATAAAC | |
| | CAAGTCCCCAAAGCATGGCGGAACA | |
| | CTCTGAAGTCCATCGAGCACAGTGT | |
| | GAAGCAGGAGGCAACACAACTACAA | |
| | TCGCTTATGAAAAAAACCTCATTGA | |
| | AATTAACAGGAGTACCCTACTTATT | |
| | TTCCAACTGTAATCCCGGGAAAACC | |
| | ACAACAGGCACTATGCCTGTATTAA | |
| | GCGAGATGGCATCAGAGCTCCTATC | |
| | AAATCCCATCTCCCAATTCCAATCA | |
| | ACATGGGGGTGTGCTGCTTCAGGGT | |
| | GGCACCATATTGTTAGCATCATGAG | |
| | GCTTCAACAGTATCAAAGAAGGACA | |
| | GGTAAAGAGGAGAAGGCGATCACTG | |
| | AGGTTCATTTTGGTTCAGACACCTG | |
| | TCTCATTAATGCAGACTACACCGTT | |
| | ATCTTTTCCTTACAGAGCCGTGTAA | |
| | TAACAGTTTTACCTTTTGACGTTGT | |
| | CCTCATGATGCAAGACCTGCTCGAA | |
| | TCTCGACGAAATGTCCTGTTCTGTG | |
| | CCCGCTTTATGTACCCCAGAAGCCA | |
| | ATTGCATGAGAGGATAAGCATGATA | |
| | CTAGCTCTCGGAGATCAACTTGGGA | |
| | AAAAGGCACCCCAAGTTCTATATGA | |
| | CTTTGTTGCAACCCTTGAATCATTT | |
| | GCATACGCAGCTGTCCAACTTCATG | |
| | ACAATAACCCTATCTACGGTGGGAC | |
| | TTTCTTTGAATTCAATATCCAAGAA | |
| | TTAGAATCTATCTTGTCTCCTGCGC | |
| | TTAGCAAGGACCAGGTCAACTTCTA | |
| | CATTAGTCAGGTTGTCTCAGCATAC | |
| | AGTAACCTCCCCCCATCTGAATCGG | |
| | CAGAATTGCTATGCCTGTTACGCCT | |
| | ATGGGGTCACCCTTTACTAAATAGC | |
| | CTCGATGCAGCAAAGAAAGTCAGAG | |
| | AATCAATGTGTGCCGGGAAGGTTCT | |
| | TGACTACAATGCCATTCGATTAGTC | |
| | TTGTCTTTTTACCATACATTATTGA | |
| | TCAATGGATATCGGAAGAAACACAA | |
| | GGGACGCTGGCCAAATGTGAATCAA | |
| | CATTCACTACTCAACCCAATAGTGA | |
| | GGCAGCTTTACTTTGATCAAGAAGA | |
| | GATCCCACATTCTGTCGCCCTCGAA | |
| | CATTACTTAGACATCTCAATGATAG | |
| | AATTTGAGAAAACTTTTGAGGTTGA | |
| | ACTATCTGACAGCCTAAGCATCTTT | |
| | TTGAAAGACAAGTCGATTGCCTTGG | |
| | ACAAACAAGAGTGGTACAGCGGTTT | |
| | TGTTTCAGAAGTGACCCCAAAGCAC | |
| | TTGCGGATGTCTCGTCATGACCGCA | |
| | AGTCCACCAACAGGCTCCTGCTGGC | |
| | CTTTATCAACTCCCCTGAATTCGAT | |
| | GTTAAAGAAGAGCTAAAATACTTGA | |
| | CTACAGGTGAGTATGCTACTGATCC | |
| | AAATTTCAACGTTTCTTACTCACTT | |
| | AAAGAGAAGGAAGTAAAGAAAGAAG | |
| | GACGAATCTTTGCAAAAATGTCACA | |
| | AAAGATGAGAGCGTGCCAGGTTATT | |
| | TGTGAAGAGTTGCTAGCACATCATG | |
| | TAGCCCCTTTGTTTAAAGAGAATGG | |
| | TGTCACACAGTCGGAACTATCTCTG | |
| | ACAAAAAATCTGCTAGCTATCAGTC | |
| | AGTTGAGTTATAACTCAATGGCTGC | |
| | TAAGGTGCGGTTGCTGAGACCAGGG | |
| | GACAAATTCACTGCCGCACACTATA | |
| | TGACCACAGACCTGAAAAAGTACTG | |
| | CCTTAATTGGCGTCACCAGTCAGTC | |
| | AAACTGTTTGCCAGAAGCCTAGATC | |
| | GACTGTTCGGGCTAGATCATGCTTT | |
| | TTCTTGGATACATGTCCGCCTCACC | |
| | AACAGCACCATGTATGTGGCTGATC | |
| | CATTCAATCCACCAGACTCAGATGC | |
| | ATGCCCAAACTTAGACGACAACAAA | |
| | AACACGGGAATTTTCATCATAAGTG | |
| | CACGAGGTGGGATAGAAGGCCTCCA | |
| | ACAAAAACTGTGGACCGGCATATCA | |
| | ATCGCAATCGCGCAAGCAGCTGCAG | |
| | CCCTCGAAGGCTTGAGAATTGCTGC | |
| | TACTTTGCAGGGGGACAACCAGGTT | |
| | CTAGCGATCACGAAGGAATTTGTAA | |
| | CCCCAGTCCCGGAAGGTGTCCTCCA | |
| | TGAGCAATTATCTGAGGCGATGTCC | |
| | CGATATAAAAAGACTTTCACATACC | |
| | TTAATTACTTAATGGGGCATCAACT | |
| | GAAAGATAAAGAGACAATCCAATCC | |
| | AGTGATTTCTTTGTTTACTCTAAAA | |
| | GGATATTCTTTAATGGGTCCATTCT | |
| | GAGTCAATGTCTCAAAAACTTCAGT | |
| | AAGCTCACCACTAATGCCACCACCC | |
| | TTGCCGAGAACACTGTAGCCGGCTG | |
| | CAGTGACATCTCATCATGCATCGCT | |
| | CGTTGTGTAGAAAACGGGTTGCCAA | |
| | AGGATGCTGCATACATCCAGAACAT | |
| | AGTCATGACTCGACTTCAACTGTTG | |
| | CTAGATCACTACTATTCCATGCATG | |
| | GTGGCATAAACTCAGAATTAGAACA | |
| | GCCGACCCTAAGTATTTCTGTTCGG | |
| | AATGCAACCTATTTACCATCTCAGT | |
| | TGGGCGGTTACAATCATCTAAATAT | |
| | GACCCGACTATTTTGCCGCAACATC | |
| | GGTGACCCGCTCACTAGTTCCTGGG | |
| | CAGAAGCAAAGAGACTAATGGAAGT | |
| | TGGCCTGCTCAATCGTAAATTCCTG | |
| | GAGGGAATATTGTGGCGACCTCCGG | |
| | GAAGTGGGACATTCTCAACACTTAT | |
| | GCTTGACCCGTTTGCGCTGAACATT | |
| | GATTACCTCAGACCACCAGAGACAA | |
| | TAATCCGAAAGCATACCCAGAAGGT | |
| | CTTGCTGCAAGATTGCCCTAATCCC | |
| | CTATTAGCCGGTGTGGTTGATCCGA | |
| | ACTACAACCAGGAACTGGAACTATT | |
| | AGCGCAGTTCTTGCTCGACCGAGAG | |
| | ACCGTTATTCCCAGGGCAGCTCATG | |
| | CTATCTTTGAGCTGTCTGTCTTGGG | |
| | GAGGAAAAAACATATACAAGGGTTG | |
| | GTGGACACTACAAAAACGATTATCC | |
| | AGTGTTCGCTGGAAAGACAACCATT | |
| | GTCCTGGAGGAAAGTTGAGAACATT | |
| | ATCACCTATAATGCGCAGTATTTCC | |
| | TTGGAGCCACTCAGCAGATTGATAC | |
| | AGATTCCCCTGAAAAGCAGTGGGTG | |
| | ATGCCAAGCAACTTCAAGAAGCTCG | |
| | TGTCTCTTGACGATTGTTCAGTCAC | |
| | ATTGTCTACTGTTTCCCGGCGTATA | |
| | TCTTGGGCCAACCTACTTAATTGGA | |
| | GGGCAATAGATGGCTTGGAAACCCC | |
| | AGATGTGATAGAAAGTATTGATGGG | |
| | CGCCTTGTGCAATCATCCAATCAGT | |
| | GTGGCCTATGTAATCAAGGATTAAG | |
| | TTCCTACTCCTGGTTCTTCCTCCCC | |
| | TCCGGATGTGTGTTTGATCGTCCAC | |
| | AAGACTCCAGGGTAGTACCGAAAAT | |
| | GCCGTATGTGGGATCCAAGACAGAT | |
| | GAGAGGCAGACTGCGTCGGTACAAG | |
| | CTATACAGGGATCCACATGTCACCT | |
| | TAGAGCAGCATTGAGACTTGTATCA | |
| | CTCTACCTTTGGGCTTATGGGGATT | |
| | CTGATATATCATGGCTGGAAGCCGC | |
| | GACACTAGCCCAAACACGGTGCAAT | |
| | ATTTCCCTTGATGATCTGCGAATCC | |
| | TGAGCCCTCTACCTTCCTCGGCAAA | |
| | TTTACACCACAGATTAAATGACGGG | |
| | GTAACACAAGTGAAATTCATGCCTG | |
| | CTACATCAAGCCGAGTATCAAAGTT | |
| | TGTCCAGATTTGCAATGACAACCAG | |
| | AATCTTATCCGTGATGATGGGAGTG | |
| | TGGATTCCAATATGATTTATCAGCA | |
| | AGTCATGATATTAGGACTTGGGGAA | |
| | TTTGAGTGCTTGTTGGCCGACCCAA | |
| | TCGATACTAACCCAGAGCAATTGAT | |
| | TCTTCATCTACACTCTGACAATTCT | |
| | TGCTGCCTCCGGGAGATGCCAACAA | |
| | CCGGCTTTGTGCCTGCTTTGGGATT | |
| | AACCCCATGCTTAACTGTACCAAAG | |
| | CAAAATCCATATATTTATGACGAGA | |
| | GTCCAATACCTGGTGACCTGGATCA | |
| | ACGGCTCATCCAAACAAAGTTTTTC | |
| | ATGGGTTCTGATAATCTAGACAACC | |
| | TTGATATCTATCAGCAACGAGCGTT | |
| | ACTAAGTCGGTGTGTGGCTTATGAT | |
| | GTTATCCAATCAGTATTTGCTTGTG | |
| | ATGCACCAGTTTCTCAGAAGAATGA | |
| | TGCAATCCTCCATACTGACTATCAT | |
| | GAGAATTGGATCTCAGAGTTCCGAT | |
| | GGGGTGACCCTCGGATAATTCAAGT | |
| | GACAGCAGGTTATGAATTGATCTTG | |
| | TTTCTTGCTTACCAGCTTTATTACC | |
| | TTAGAGTGAGGGGTGACCGTGCAAT | |
| | CCTGTGCTATATTGATAGGATACTG | |
| | AATAGGATGGTGTCATCAAATCTAG | |
| | GCAGCCTTATCCAGACACTCTCCCA | |
| | TCCGGAGATTAGGAGGAGGTTTTCA | |
| | TTAAGTGATCAAGGATTCCTTGTTG | |
| | AAAGGGAACTAGAGCCAGGCAAACC | |
| | TTTGGTAAAACAAGCAGTCATGTTC | |
| | CTAAGGGACTCAGTCCGATGTGCTT | |
| | TAGCAACTATCAAGGCAGGAGTCGA | |
| | GCCGGAGATCTCCCGAGGTGGCTGT | |
| | ACCCAAGATGAGTTGAGTTTCACCC | |
| | TCAAGCACTTGCTATGTCGACGTCT | |
| | CTGTATAATTGCTCTCATGCATTCA | |
| | GAAGCAAAGAACTTGGTCAAGGTCA | |
| | GAAATCTCCCAGTAGAGGAAAAATC | |
| | TGCTTTACTATACCAGATGTTGGTC | |
| | ACCGAAGCTAATGCCCGGAAATCAG | |
| | GATCTGCTAGCATCATCATAGGCTT | |
| | AATTTCGGCACCTCAGTGGGATATC | |
| | CATACCCCAGCACTGTACTTTGTAT | |
| | CAAAGAAGATGCTAGGAATGCTCAA | |
| | AAGGTCAACTACACCATTGGATGTA | |
| | AATGATCTGTCTGAGAGCCAGGACC | |
| | TTATGCCAACAGAGTTGAGTGATGG | |
| | TCCTGGTCACATGGCAGAGGGATTT | |
| | CCCTGTCTATTTAGTAGTTTTAACG | |
| | CTACATATGAAGACACAATTGTTTA | |
| | TAATCCGATGACTGAAAAGCCTGCA | |
| | GTACATTTGGACAATGGATCCACCC | |
| | CATCCAGGGCGCTAGGTCGCCACTA | |
| | CATCTTGCGGCCCCTCGGGCTTTAC | |
| | TCGTCTGCATGGTACCGGTCTGCAG | |
| | CACTCTTAGCATCAGGTGCTCTCAA | |
| | TGGGTTACCGGAGGGATCAAGCCTA | |
| | TACTTGGGAGAAGGGTATGGGACCA | |
| | CCATGACTCTGCTCGAACCCGTCGT | |
| | CAAGTCCTCAACTGTTTATTACCAC | |
| | ACATTGTTTGACCCGACCCGGAATC | |
| | CCTCACAGCGGAATTACAAACCAGA | |
| | GCCGCGAGTCTTCACTGATTCCATC | |
| | TGGTACAAGGATGACTTCACACGAC | |
| | CGCCTGGTGGCATTGTAAATCTATG | |
| | GGGTGAAGATGTGCGTCAGAGTGAC | |
| | GTCACACAGAAAGACACAGTTAATT | |
| | TCATATTATCCCGGATCCCACCCAA | |
| | ATCACTCAAACTGATCCATGTTGAC | |
| | ATTGAATTCTCACCAGACTCCAATG | |
| | TACGGACACTACTATCTGGTTACTC | |
| | CCATTGCGCATTATTGGCCTACTGG | |
| | CTATTGCAACCTGGAGGGCGATTTG | |
| | CGGTTAGGGTCTTCCTGAGTGACCA | |
| | TCTCTTAGTAAACTTGGTCACTGCT | |
| | ATTCTGTCTGCTTTCGACTCTAATC | |
| | TACTGTGTATTGCATCTGGATTGAC | |
| | ACACAAAGATGATGGGGCAGGTTAC | |
| | ATTTGTGCTAAGAAGCTTGCCAATG | |
| | TTGAGGCATCAAGGATTGAGCACTA | |
| | CTTAAGGATGGTCCATGGTTGCGTT | |
| | GATTCATTAAAGATCCCCCACCAAC | |
| | TAGGGATCATTAAGTGGGCTGAAGG | |
| | TGAGGTGTCTCGGCTCACAAAAAAG | |
| | GCAGATGAAGAAATAAATTGGCGAT | |
| | TAGGTGACCCGGTTACTAGATCATT | |
| | TGATCCAGTTTCCGAGTTAATAATC | |
| | GCACGGACAGGGGGGTCTGTATTAA | |
| | TGGAATATGGGACTTTCATTAATCT | |
| | CAGGTGTTCAAACCTGGCAGATACA | |
| | TATAAACTTTTGGCTTCAATCGTGG | |
| | AGACCACCTTGATGGAGATAAGGGT | |
| | TGAACAAGATCAATTGGAAGACAAC | |
| | TCAAGAAGACAAATTCAGGTGGTCC | |
| | CCGCCTTTAATACGAGATCCGGGGG | |
| | GAGGATCCGTACATTGATTGAGTGT | |
| | GCCCAGCTGCAGGTTATAGATGTCA | |
| | TATGTGTAAACATAGATCACCTCTT | |
| | CCCCAAACATCGACATGTTCTTGTT | |
| | ACACAACTCACTTACCAGTCAGTGT | |
| | GCCTTGGAGACTTGATCGAGGGGCC | |
| | CCAAATTAAGATGTATCTAAGGGCC | |
| | AGGAAGTGGATCCAACGTAGAGGAC | |
| | TCAATGAGACAATTAACCATATCAT | |
| | CACTGGACAGATATCACGAAATAAG | |
| | GCAAGGGATTTCTTCAAGAGGCGCC | |
| | TGAAGTTGGTTGGCTTCTCGCTTTG | |
| | CGGCGGTTGGAGTTACCTCTCACTT | |
| | TAGTTACTTAGGTTGTTGATCATTG | |
| | TGAAAAATCGGAGTCGGAATCGCAA | |
| | ATAAAAACATACAAAATTGCAAATT | |
| | TACAATAATCGCATTAATATTTAAT | |
| | AAAAAATATGTCTTTTATTTCGT | |
| Avian | ACCAAACAAGGAAACCATATGCTTG | SEQ ID |
| paramyxovir | GGGACTTTACGAGAGCGCTTGTAAA | No: 9 |
| us 6 strain | ACCGTGAGGGGGAAGCTGGTGGACT | |
| APMV- | CCGGGTCCGGAGTCGGTGGACCTGA | |
| 6/duck/ | GTCTAGTAGCTTCCCTGCTGTGTCA | |
| HongKong/ | AGATGTCGTCAGTGTTCACTGATTA | |
| 18/199/77, | CGCTAAGCTGCAAGATGCCCTTGTG | |
| complete | GCCCCTTCGAAGAGGAAGGTAGATA | |
| genome | GTGCACCAAGCGGATTGTTAAGGGT | |
| Genbank: | TGGGATCCCTGTGTGTGTCCTACTC | |
| EU622637.2 | TCCGAAGATCCCGAAGAGCGATGGA | |
| | GCTTCGTTTGCTTTTGCATGAGATG | |
| | GGTGGTGAGCGATTCAGCCACAGAA | |
| | GCGATGCGTGTTGGTGCAATGCTAT | |
| | CCATTCTCAGCGCACACGCCAGCAA | |
| | TATGCGGAGCCACGTTGCACTTGCA | |
| | GCGAGGTGTGGTGACGCCGACATCA | |
| | ACATACTTGAGGTTGAGGCAATTGA | |
| | CCACCAGAACCAGACCATTCGCTTC | |
| | ACTGGGCGCAGCAATGTGACTGACG | |
| | GGAGAGCACGCCAGATGTACGCAAT | |
| | TGCCCAAGATTTGCCTCCTTCCTAT | |
| | AACAATGGCAGCCCTTTTGTAAATA | |
| | GAGACATTGAGGACAATTATCCAAC | |
| | TGACATGTCTGAGCTGCTCAATATG | |
| | GTTTACAGTGTCGCAACTCAAATCT | |
| | GGGTGGCAGCTATGAAGAGCATGAC | |
| | TGCTCCAGACACATCCTCGGAGTCT | |
| | GAGGGGAGGCGGCTGGCCAAATACA | |
| | TCCAGCAAAACAGAGTAATTCGGAG | |
| | CACGATTCTAGCTCCCGCAACCCGC | |
| | GGTGAATGCACCCGAATAATACGGA | |
| | GCTCCCTAGTCATCCGCCACTTCCT | |
| | AATAACTGAGATCAAGCGTGCCACA | |
| | TCAATGGGTTCCAACACGACACGAT | |
| | ATTATGCCACAGTTGGGGATGCCGC | |
| | AGCTTACTTCAAGAATGCGGGTATG | |
| | GCTGCATTCTTCTTAACTCTGAGGT | |
| | TTGGAATTGGGACCAAGTACTCCAC | |
| | ACTTGCAGTTTCGGCGCTGTCTGCT | |
| | GACATGAAGAAACTCCAGAGCTTGA | |
| | TCCGAGTATACCAGAGCAAAGGTGA | |
| | GGATGGACCCTACATGGCATTTCTG | |
| | GAAGACTCCGACCTTATGAGCTTCG | |
| | CCCCTGGAAACTATCCACTCATGTA | |
| | TTCATATGCAATGGGAGTAGGGTCC | |
| | ATTCTTGAGGCAAGTATTGCTAGAT | |
| | ATCAGTTTGCGCGATCATTCATGAA | |
| | TGACACATTCTATCGATTGGGTGTT | |
| | GAAACTGCACAACGAAACCAAGGTT | |
| | CACTTGATGAGAATTTAGCAAAGGA | |
| | GCTGCAACTATCCGGGGCTGAACGA | |
| | AGGGCTGTGCAGGAACTTGTGACCA | |
| | GCCTGGATCTAGCAGGAGAGGCCCC | |
| | AGTGCCCCAGCGCCAACCAACATTC | |
| | CTCAATGACCAGGAGTATGAGGATG | |
| | ATCCCCCTGCTAGGAGACAGAGAAT | |
| | CGAGGATACTCCAGACGATGATGGA | |
| | GCCAGTCAAGCTCCACCCACACCAG | |
| | GAGCAGGTCTCACCCCATACTCTGA | |
| | TAATGCCAGTGGCCTGGACATCTAA | |
| | ATGACCACTACTCAATATGACAAGT | |
| | AATCAAGGTTGATCCAAAGCATGCA | |
| | AATCCAACACTACAATCGACAACAA | |
| | AATCACATGTAGACTTTAAGAAAAA | |
| | ACAAGGGTGAGGGGGAAGTTCCTGG | |
| | TGCGCGGGTTGGGCCCCTAGTGACT | |
| | CAGCCAGCACCATGGACTTCTCCAA | |
| | TGACCAAGAGATTGCAGAATTACTC | |
| | GAGCTGAGTTCAGATGTGATAAAGA | |
| | GCATCCAACACGCCGAGACCCAGCC | |
| | AGCGCACACTGTCGGCAAATCTGCC | |
| | ATTCGGAAAGGAAACACATCCGAGC | |
| | TGCGAGCAGCCTGGGAAGCCGAGAC | |
| | ACAACCAGCCCGAGCAGAAAACAAG | |
| | CCCGAGGAACACCCAGAGCAAGCCG | |
| | CCCGGGATCTCGACAGCAAGGGCAA | |
| | CACGGAAAGCCCACAACTACGATCC | |
| | AATGCAGATGAGACACCCCAACCAG | |
| | AAAGCCACGACAGGCAAGCCACTGC | |
| | CCCATCCCCAGACACCACAATAGGG | |
| | GTCAACGGGACTAATGGACTTGAAG | |
| | CTGCTCTAAAAAAGCTAGAAAAACA | |
| | AGGGAAAGGTCCTGGGAAAGGCCAA | |
| | GTGGATCGCAACACTCCTCAGAGAG | |
| | ATCCAACCACTGCTTCGGGTTCAAA | |
| | AAAGGGGAAAGGGGGCGAGCCAAGG | |
| | AACAATGCCCTTCATCAGGGCCACC | |
| | CACAGGGGACCAACCTGATCCTGCC | |
| | CACTCAGAAGCCCTCTCATGCCAGA | |
| | CTGGCGCAGCAAGCATCACAGGAGA | |
| | TAACTCGCCATGCACTGCAACCCCA | |
| | GGATTCCGGCGGCATAGAAGGGAAT | |
| | TCTCCATTTCTTGGAGACACGGCCA | |
| | GTGCATCTTGGCTGAGTGGTGCAAC | |
| | CCAGTCTGCGCACCCGTCACACCTG | |
| | AACCCAGAACATTCAAATGCATTTG | |
| | CGGGAGATGCCCTCGGGTATGCATC | |
| | AACTGTCGCAATGATAGTGGAGACT | |
| | CTGAAATTTGTAGTTAGCAGGTTAG | |
| | AAGCACTTGAGAATAGGGTGGCGGA | |
| | GCTTACCAAGTTTGTCTCTCCCATT | |
| | CAGCAAATCAAAGCAGACATGCAGA | |
| | TTGTAAAGACATCCTGCGCTGTCAT | |
| | TGAGGGCCAACTTGCCACAGTGCAA | |
| | ATATTGGAGCCGGGCCACTCATCGA | |
| | TCCGCTCACTTGAAGAAATGAAGCA | |
| | ATATACCAAGCCAGGGGTTGTCGTC | |
| | CAAACAGGGACGACTCAAGACATGG | |
| | GCGCCGTCATGAGGGACGGCACGAT | |
| | CGTGAAAGATGCTCTTGCCCGCCCA | |
| | GTCAATCCGGACAGGTGGTCAGCAA | |
| | CAATCAACGCTCAATCAACAACAAC | |
| | AAAGGTGACTCAAGAGGATATAAAG | |
| | ACAGTGTATACACTATTGGACAATT | |
| | TTGGCATCACCGGCCCGAAAAGAGC | |
| | GAAAATCGAGGCAGAACTGGCTAAT | |
| | GTCAGTGACCGGGACGCACTAGTAA | |
| | GGATAAAGAAACGTGTTATGAATGC | |
| | ATAAACAGCAAGAAGATCACAACAA | |
| | TCAGTACAGATGACATCCCAATATC | |
| | AGATCATGATTCTATTGCCAAATCA | |
| | CAGCATTTTTTTCTCCTGATCACAC | |
| | CTAACAATTTGCTTCAGACACCCTT | |
| | GACACTGATTAATAAAAAAGTGAGG | |
| | GGGAACTGGTGGTGTCCGGACTGGG | |
| | CCATCCAGAGTCACCCAGTCCGAAC | |
| | CAAACACCCGCCAGTTCCTCCGCCG | |
| | GCACAGCGCGCCACCAACTGCCCCA | |
| | ACTCCAACCATGGCCACATCAGAAC | |
| | TCAACCTCTACATCGACAAAGACTC | |
| | ACCCCAGGTGAGATTGCTAGCATTC | |
| | CCCATCATCATGAAACCCAAAGAAA | |
| | GTGGGGTTAGAGAGCTGCAACCGCA | |
| | ATTGAGGACCCAGTACCTCGGTGAC | |
| | GTTACCGGAGGAAAGAAAAGCGCGA | |
| | TATTTGTGAATTGCTATGGGTTCGT | |
| | GGAAGATCACGGGGGGCGAGACAGC | |
| | GGATTCTCACCCATCAGCGAGGAAT | |
| | CCAAAGGATCGACAGTCACTGCAGC | |
| | TTGCATCACTCTCGGCAGCATCGAG | |
| | TATGATAGTGACATCAAGGAGGTGG | |
| | CAAAGGCCTGCTATAATCTTCAGGT | |
| | GTCAGTCAGGATGTCCGCTGATTCA | |
| | ACTCAGAAGGTAGTTTACACAATCA | |
| | ATGCCAAACCTGCACTGTTGTTCTC | |
| | CTCCCGTGTTGTCAGGGCTGGGGGT | |
| | TGTGTGGTTGCAGCAGAAGGTGCAA | |
| | TCAAGTGCCCCGAGAAAATGACATC | |
| | TGATCGCCTCTACAAATTCCGCGTA | |
| | ATGTTTGTGTCATTGACCTTCCTAC | |
| | ATCGCAGCAGCCTTTTTAAAGTTAG | |
| | CCGTACAGTGCTGTCAATGAGGAAT | |
| | TCTGCTCTAATAGCAGTACAGGCCG | |
| | AAGTGAAGCTGGGGTTCGATCTGCC | |
| | ACTGGACCATCCGATGGCAAAATAT | |
| | TTGAGCAAAGAGGATGGACAGCTAT | |
| | TTGCAACTGTGTGGGTACACTTGTG | |
| | CAACTTTAAGCGCACAGACAGACGC | |
| | GGAGTAGACCGATCGGTGGAGAACA | |
| | TCAGGAACAAAGTACGAGCCATGGG | |
| | GCTGAAGCTCACCTTGTGTGATCTA | |
| | TGGGGTCCCACACTTGTTTGTGAAG | |
| | CCACGGGGAAGATGAGCAAGTACGC | |
| | GCTAGGTTTCTTCTCGGAGACTAAG | |
| | GTTGGCTGTCACCCAATCTGGAAAT | |
| | GCAACTCGACTGTCGCAAAGATCAT | |
| | GTGGTCATGCACAACTTGGATCGCA | |
| | TCAGCAAAGGCCATCATACAGGCCT | |
| | CCTCTGCTCGTACCTTGTTGACATC | |
| | AGAGGACATAGAAGCCAAGGGGGCC | |
| | ATCTCCACTGACAAGAAGAAAACAG | |
| | ATGGATTCAATCCCTTCATCAAGAC | |
| | AGCAAAGTAGTCATCTGGATTTCAT | |
| | CAATGAACCCACTGGCCTATGTTCA | |
| | GCTGTACCTTCCTTGATAATCACTA | |
| | AATCAATACACAGAGTGCCATTTGA | |
| | TTAAGATATTGATTGTGCCAGTATG | |
| | TGGATCACTTATACTTTGAAGATTG | |
| | ACCTTCCTAGCTGTTCCTCCCTTAG | |
| | AAGTCCTGTCATATTAATCAAAAAA | |
| | ATCAGTTTGCTGGTAAAATAGTATG | |
| | CTGCAGGATCCAATACCTCCCACCA | |
| | ATGAGCAGCCGAGGGGGAAGGCATG | |
| | GGAGCCCGACTGGGGCCCTTTACAA | |
| | TGGCACCCGGCCGGTATGTGATTAT | |
| | TTTCAACCTCATCCTTCTCCACAAG | |
| | GTTGTGTCACTAGACAATTCAAGAT | |
| | TACTACAGCAGGGGATTATGAGTGC | |
| | AACCGAAAGAGAAATCAAAGTGTAC | |
| | ACAAACTCCATAACTGGAAGCATTG | |
| | CTGTGAGATTGATTCCCAACCTACC | |
| | TCAAGAAGTGCTTAAATGTTCTGCT | |
| | GGGCAGATCAAATCATACAATGACA | |
| | CCCTTAATCGAATTTTCACACCTAT | |
| | CAAGGCGAATCTTGAGAGGTTACTG | |
| | GCTACACCGAGTATGCTTGAACACA | |
| | ACCAGAACCCTGCCCCAGAACCTCG | |
| | CCTGATTGGAGCAATTATAGGCACA | |
| | GCAGCACTGGGGCTGGCAACAGCAG | |
| | CTCAGGTTACAGCTGCACTCGCCCT | |
| | TAACCAGGCCCAGGATAATGCTAAG | |
| | GCCATCTTAAACCTCAAAGAGTCCA | |
| | TAACAAAAACAAATGAAGCTGTGCT | |
| | TGAGCTTAAGGATGCAACAGGGCAA | |
| | ATTGCGATAGCGCTAGATAAGACTC | |
| | AAAGATTCATAAATGACAATATCTT | |
| | ACCGGCAATCAATAATCTGACATGT | |
| | GAAGTAGCAGGTGCTAAAGTAGGTG | |
| | TGGAACTATCATTATACTTGACCGA | |
| | GTTAAGCACTGTGTTTGGGTCGCAG | |
| | ATAACCAATCCAGCACTCTCCACTC | |
| | TATCCATTCAAGCCCTCATGTCACT | |
| | CTGCGGTAATGATTTTAATTACCTC | |
| | CTGAACCTAATGGGGGCCAAACACT | |
| | CCGATCTGGGTGCACTTTATGAGGC | |
| | AAACTTAATCAATGGCAGAATCATT | |
| | CAATATGACCAAGCAAGCCAAATCA | |
| | TGGTTATCCAGGTCTCCGTGCCTAG | |
| | CATATCATCGATTTCGGGGTTGCGA | |
| | CTGACAGAATTGTTTACTCTGAGCA | |
| | TTGAAACACCTGTCGGTGAGGGCAA | |
| | GGCAGTGGTACCTCAGTTTGTTGTA | |
| | GAATCTGGCCAGCTTCTTGAAGAGA | |
| | TCGACACCCAGGCATGCACACTCAC | |
| | TGACACCACCGCTTACTGTACTATA | |
| | GTTAGAACAAAACCATTGCCAGAAC | |
| | TAGTCGCACAATGTCTCCGAGGGGA | |
| | TGAGTCTAGATGCCAATATACGACT | |
| | GGAATCGGTATGCTTGAATCTCGAT | |
| | TTGGGGTATTTGATGGACTTGTTAT | |
| | TGCTAATTGTAAGGCCACCATCTGC | |
| | CGATGTCTAGCCCCTGAGATGATAA | |
| | TAACTCAAAACAAGGGACTCCCCCT | |
| | TACAGTCATATCACAAGAAACTTGC | |
| | AAGAGAATCCTGATAGATGGGGTTA | |
| | CTCTGCAGATAGAAGCTCAAGTTAG | |
| | CGGATCGTATTCCAGGAATATAACG | |
| | GTCGGGAACAGCCAAATTGCCCCAT | |
| | CTGGACCCCTTGACATCTCAAGCGA | |
| | ACTCGGAAAGGTCAACCAGAGTCTA | |
| | TCTAATGTCGAGGATCTTATTGACC | |
| | AGAGCAATCAGCTCTTGAATAGGGT | |
| | GAATCCAAACATAGTAAACAACACC | |
| | GCAATTATAGTCACAATAGTATTGC | |
| | TAGTTATCCTGGTATTATGGTGTTT | |
| | GGCCCTAACGATTAGTATCTTGTAT | |
| | GTATCAAAACATGCTGTGCGAATGA | |
| | TAAAGACAGTTCCGAATCCGTATGT | |
| | AATGCAAGCAAAGTCGCCGGGAAGT | |
| | GCCACACAGTTCTAACAGTATAGCT | |
| | AGTCCTAATGATTAAACCATATACT | |
| | TGATTACATAATAACACTATGTCAA | |
| | GGGATGACATTAATGAGACTCCTTA | |
| | TTCTCTCTCAAACCGAGACAGTGAT | |
| | CCATCAAGAATGCAACGATCCTACC | |
| | TTCTCTGCTTTAATCAAAAAATGCA | |
| | GAATAATCTAACAGCCCAACCAAAC | |
| | CACCCAGGAGAGAACGCCTGAGGGG | |
| | GGAAGGAGGTTGACTACAACCTCTA | |
| | CTGATCAGAGGTTGTAGTATCAATT | |
| | CTTAACAACCCCCAAGATGAGACCA | |
| | CAAGTGGCAATTTGGGGCTTGCGCT | |
| | TATTGGCTACCGGCCTAGCTATGGT | |
| | CTCCTTAGTGTTCTGCCTAAACCAG | |
| | GTAATCATGCAGGTGCTAATTAGGG | |
| | ACATTAGAGGCTTGTTGACATCCTC | |
| | GGACATCAAGACTACACATGAGGCG | |
| | CTGCGTGAGCATCTCTCATCTATTA | |
| | CTCTTTTCATGTCGTTTGCGTTGAC | |
| | TTGCTCAATAAGTGGGTGTGTTCTT | |
| | AGCCTGGTCGCCTTATATCCAAGCA | |
| | AGAATACTAGCGGCACTAATCCTCA | |
| | GCCGCAAGTAGAGGAGGCTAGATCG | |
| | GAAAACCTGTCTCACTCTTCCATGC | |
| | ACACGATCAATAGGCCAGCAACCCC | |
| | TCCCCCACCGTATTATGTTGCAATA | |
| | CAGCTCAGCGCTGAGATGCAACCTG | |
| | GGTACCATTCAAGTGATTGATCCCC | |
| | TTGACGCACTGGCAGAGTCTACCCC | |
| | ACCAAGATCCGTTCTTGTCCTACTT | |
| | GTTTGATTTAAGAAAAAATTGTAAT | |
| | TTATACAGAAAGATAATAGCTGAGG | |
| | GGGAAGCCTGGTGTCACCGCTGGTG | |
| | ACCATTCCCCAGCCGGTGGCAATGG | |
| | CTTCCTCAGGCGATATGAGACAGAG | |
| | TCAGGCAACTCTATATGAGGGTGAC | |
| | CCTAACAGCAAAAGGACATGGAGGA | |
| | CTGTGTACCGGGTTGTCACCATATT | |
| | GCTAGATATAACCGTCCTTTGTGTT | |
| | GGCATAGTGGCAATAGTTAGGATGT | |
| | CAACCATTACAACAAAAGATATTGA | |
| | TAACAGTATCTCATCATCTATTACA | |
| | TCCCTGAGTGCCGATTACCAGCCAA | |
| | TATGGTCAGATACCCATCAGAAAGT | |
| | TAACAGTATTTTCAAGGAAGTTGGA | |
| | ATCACTATCCCTGTCACACTCGACA | |
| | AGATGCAAGTAGAAATGGGAACAGC | |
| | GGTTAACATAATCACTGATGCTGTA | |
| | AGACAACTACAAGGAGTCAATGGGT | |
| | CAGCAGGATTTAGCATTACCAATTC | |
| | CCCAGAGTATAGTGGAGGGATAGAC | |
| | ACACTGATATACCCTCTTAATTCAC | |
| | TTAATGGAAAGGCTCTAGCTGTATC | |
| | AGACTTACTAGAACACCCGAGCTTC | |
| | ATACCGACGCCTACCACCTCTCACG | |
| | GTTGTACCCGCATTCCTACATTCCA | |
| | CCTAGGGTACCGTCATTGGTGTTAT | |
| | AGTCACAACACGATAGAGTCTGGTT | |
| | GTCACGATGCAGGAGAAAGCATTAT | |
| | GTACGTATCCATGGGTGCGGTAGGG | |
| | GTCGGCCATCGCGGGAAACCTGTGT | |
| | TTACGACAAGTGCAGCGACAATCCT | |
| | AGATGATGGAAGGAACAGGAAAAGT | |
| | TGTAGCATCATAGCAAACCCTAATG | |
| | GGTGTGATGTCTTATGCAGCTTGGT | |
| | TAAGCAGACAGAAAATGAAGGCTAC | |
| | GCTGACCCTACACCGACCCCAATGA | |
| | TCCACGGTAGGCTCCACTTCAATGG | |
| | CACATACACTGAGTCTGAACTTGAC | |
| | CCTGGCCTATTTAATAACCATTGGG | |
| | TCGCTCAATATCCAGCAGTTGGTAG | |
| | CGGTGTCGTCAGCCACAGAAAACTA | |
| | TTTTTCCCGCTCTACGGAGGGATAT | |
| | CACCGAAGTCAAAACTGTTCAATGA | |
| | GCTCAAGTCATTTGCTTACTTTACT | |
| | CATAATGCTGAATTGAAATGTGAGA | |
| | ACCTGACAGAGAGACAGAAGGAAGA | |
| | CCTTTATAACGCATATAGGCCTGGG | |
| | AAAATAGCAGGATCTCTCTGGGCTC | |
| | AAGGGGTTGTAACATGTAATCTGAC | |
| | CAATTTAGCTGATTGCAAAGTTGCA | |
| | ATTGCGAACACGAGCACCATGATGA | |
| | TGGCTGCCGAGGGGAGGTTACAGCT | |
| | TGTGCAAGATAAGATTGTCTTCTAC | |
| | CAAAGATCCTCATCATGGTGGCCAG | |
| | TCCTAATATATTATGATATCCCTAT | |
| | TAGTGACCTTATCAGTGCCGATCAT | |
| | TTAGGGATAGTGAACTGGACTCCGT | |
| | ATCCACAGTCTAAGTTTCCGAGGCC | |
| | CACCTGGACAAAGGGCGTATGTGAG | |
| | AAACCGGCGATATGCCCCGCTGTAT | |
| | GTGTAACGGGTGTTTACCAAGATGT | |
| | TTGGGTAGTTAGTATAGGGTCACAG | |
| | AGCAATGAGACTGTTGTGGTTGGCG | |
| | GGTACTTAGATGCTGCAGCAGCCCG | |
| | TCAGGATCCATGGATTGCAGCAGCT | |
| | AACCAGTACAACTGGCTGGTTAGGC | |
| | GTCGCCTCTTTACATCCCAAACTAA | |
| | AGCAGCATACTCATCAACCACTTGC | |
| | TTCAGAAACACGAAGCAGGATAGAG | |
| | TGTTCTGCCTGACTATAATGGAAGT | |
| | CACAGACAACCTACTCGGAGACTGG | |
| | AGGATCGCCCCGCTGTTGTATGAAG | |
| | TTACTGTGGCTGATAAGCAGCAGGG | |
| | CAATCGCAATTACGTGCCTATGGGG | |
| | AGGGTGGGGACAGATAAGTTCCAAT | |
| | ATTATACCCCAGGTGACAGATATAC | |
| | TCCTCAGCATTGATGACTCACTGCA | |
| | GCTTATACATAACAATTTTCTCATT | |
| | TCCTCTATTCGCAGAGTGAATCAGT | |
| | AGAATGACGGTCAGTGATTGACCAA | |
| | GCTCAATTAGATAATGAAGTGCAGC | |
| | CCGCAATTGTCTTGATTTAATAAAA | |
| | AATTGAGGGGCTGTTATAACATAGC | |
| | AGACTGACGGGGCAAGACCCGCTGA | |
| | GAAAAAAAATGCAGTGAGGGGGAAG | |
| | GCAGGCTGAGATCACGTCCCAGTTG | |
| | TAGCCTTCCCCGATTCAATTTACTT | |
| | AGTATTAACAAGTCAATTCTGCTCA | |
| | CAGAGGTCATCTCTAAGGGCCGCTG | |
| | TGATGGATCCACAAGTCCAAATACA | |
| | CCATATCATCAAGCCAGAGTGCCAT | |
| | CTCAACTCACCTGTTGTGGAAAAGA | |
| | AACTGACATTATTATGGAAGCTCAC | |
| | AGGTTTACCGTTGCCACCCGACCTT | |
| | AACGGTTGCGTCACACACAAAGACG | |
| | TGACGTGGGATGAAGTGCTCCGGTT | |
| | GGAGGCTAATTTGACGAAGGAGTTA | |
| | CGGCAATTAGTACGAAGCCTGACCA | |
| | ATAGAATGCATGAAAAGGGGGAGTT | |
| | CATTGACACATATAAACCTTTATGT | |
| | CATCCACGGACATTAAGTTGGTTGA | |
| | CCAATATCAACTTGATCAAGAGTGA | |
| | CAACATTCTAGCAAGCCACAAGAAA | |
| | ATGTTGATCCGAATCGGCAGTATGC | |
| | TGCATGAACCAACAGACCAATCGTT | |
| | TGTCACTCTTGGCAGGAAATTAGCA | |
| | GGCGACCCTTGCTTGTTCCATCAAC | |
| | TAGGCCATCTACCTGGATGCCCACC | |
| | TAATTCCAGATTTGAAGAACAGGTA | |
| | GGAGACTGCAGTTTGTGGTCACCCA | |
| | TAAGCGATCCAGCTCTAGTCACAGG | |
| | TGGTGAATACGCTAACTGTGTGTAT | |
| | GCGTGGTACTTAATACGTCAGACCA | |
| | TGCGGTACATGGCCCTCCAGAGAAA | |
| | GCAAACAAGAGTGCAATCACAGCAG | |
| | AATGTTCTAATTGGATCAGATACTA | |
| | TCGTGGGAATCCATCCAGAATTAGT | |
| | GATAATTACTGGAATTAGAGACAGG | |
| | GTATTCACCTGTTTGACTTTTGATA | |
| | TGGTGCTAATGTATGCAGATGTGGT | |
| | GGAAGGTCGTGCCATGACAAAGTTG | |
| | GTTGCACTCACTGAGCCAACAATGG | |
| | TAGAAGTCATTCAGAGAGTCGAAAA | |
| | ATTGTGGTTCTTAGTTGACAACATC | |
| | TTCGAGGAAATCGGTGGTGCAGGTT | |
| | ACAATATTGTTGCATCTCTGGAGAG | |
| | CTTGGCATATGGTACTGTTCAACTG | |
| | TGGGATAAATCACTGGAACATGCTG | |
| | GTGAGTTCTTTTCATTCAATCTTAC | |
| | CGAGATAAAGAGTGAGCTAGAGAAC | |
| | CATTTAGATCCTGGTATGGCATTTA | |
| | GAGTAGTCGAGCAGGTGCGGTTGCT | |
| | ATATACTGGACTAAGTGTGAACCAA | |
| | GCAGGTGAGATGTTATGCATTTTAC | |
| | GTCACTGGGGGCATCCCTTACTATG | |
| | CGCTGTGAAGGCGGCAAAGAAAGTC | |
| | AGAGAGTCAATGTGTGCACCAAAAT | |
| | TAACCTCTCTAGACACCACACTCAA | |
| | GGTGTTAGCATTCTTTATTGCAGAT | |
| | ATCATCAATGGACATAGACGATCAC | |
| | ATTCAGGGTTATGGCCAAGCGTCAG | |
| | ACAGGAGTCATTAGTGTCTCCATTG | |
| | CTCCAGAACCTCTATAGAGAATCTG | |
| | CCGAGCTTCAATACGCAGTTGTGCT | |
| | TAAGCACTATAGAGAAGTATCCCTT | |
| | ATAGAATTCCAAAAAAGTATTGATT | |
| | TTGACTTAGTTGAAGATCTAAGTGT | |
| | GTTCCTTAAGGATAAAGCCATTTGT | |
| | CGACCGAAGAGTAACTGGTTAGCTG | |
| | TATTCAGGAAATCCCTACTCCCTGG | |
| | ACATTTGAAAGATAAACTGCAATCT | |
| | GAGGGCCCTTCTAACCGGCTTCTGC | |
| | TTGACTTTTTGCAATCAAGCGAATT | |
| | TGACCCGGCTAAAGAATTCGAATAC | |
| | GTGACATCGCTGGAGTATCTTCAGG | |
| | ATCCAGAGTTCTGCGCATCTTATTC | |
| | CTTAAAAGAGCGGGAAGTCAAAACT | |
| | GATGGGCGCATATTTGCAAAAATGA | |
| | CTAGAAAAATGAGGAACTGCCAAGT | |
| | CTTGTTAGAGAGTCTGCTCGCATGC | |
| | CATGTATGCGATTACTTCAAGGAGA | |
| | ACGGAGTAGTACAAGAGCAAATCAG | |
| | TTTAACAAAATCACTGCTTGCAATG | |
| | TCGCAACTTGCTCCTCGTGTGTCTG | |
| | AGTATCAAGGGAGAGTTCTCCGCTC | |
| | GACTGATAGGTGCAGTAGAGCTACA | |
| | GCCACACCTAGTCAGGACACAGGCC | |
| | CAGGCGAGGGGGTCAGGCGACGGAA | |
| | AACAATTATAGCATCATTCTTGACT | |
| | ACTGACCTACAGAAGTATTGTCTCA | |
| | ATTGGAGGTACACCGTAATAAAACC | |
| | TTTTGCCCAGAGGCTTAACCAGTTA | |
| | TTTGGGATACCCCACGGCTTTGAGT | |
| | GGATTCACCTCCGCTTGATGAACAC | |
| | AACTATGTTTGTAGGAGACCCACAT | |
| | AATGTCCCTCAGTTTTCATCGACAC | |
| | ACGACTTAGAATCCCAAGAGAACGA | |
| | TGGAATATTTATTGTGTCACCTCGG | |
| | GGTGGTATAGAAGGGCTATGCCAAA | |
| | AAATGTGGACCATGATCTCCATTGC | |
| | GGCAATTCATCTAGCAGCCACAGAA | |
| | TCGGGTTGTCGGGTTGCATCCATGG | |
| | TCCAGGGGGACAACCAAGCAATTGC | |
| | AATTACTACGGAGATCGAAGAGGGT | |
| | GAGGACGCGTCTGTAGCATCAATAA | |
| | GGTTGAAAGAGATATCTGAGAGGTT | |
| | CTTTAGGGTGTTCAGAGAGATCAAC | |
| | AGGGGTATAGGACACAACTTAAAAG | |
| | TCCAAGAAACAATTCATAGTGAGTC | |
| | ATTCTTCGTGTACTCAAAACGGATC | |
| | TTCTTTGAGGGGAAGATCCTCAGCC | |
| | AGCTACTGAAAAATGCAAGCAGGTT | |
| | GGTGTTGGTATCCGAGACTGTGGGT | |
| | GAGAATTGTGTTGGCAATTGCTCAA | |
| | ATATCAGTTCCACAGTTGCTAGACT | |
| | CATTGAAAATGGATTAGATAAGAGA | |
| | GTCGCATGGGGGCTCAATATCCTGA | |
| | TGATCGTAAAACAAATTCTTTTTGA | |
| | CATTGATTTTTCCTTGGAGCCTGAA | |
| | CCATCTCAGGGCTTGAGTCATGCTA | |
| | TTCGCCAAGACCCAAACAACATGAA | |
| | AAACATCTCTATCACTCCTGCTCAG | |
| | TTAGGTGGATTAAATTTTCTGGCCC | |
| | TATCTCGGCTATTTACAAGGAACAT | |
| | AGGAGACCCCGTCTCATCAGCCATG | |
| | GCAGATATGAAGTTCTATATACAGG | |
| | TCGGATTATTATCCCCTCATCTGCT | |
| | GAGGAATGCAATTTTCAGAGAACCC | |
| | GGAGATGGAACATGGACAACACTGT | |
| | GTGCCGACCCGTACTCATTAAACCA | |
| | ACCATATGTGCAATTACCAACGTCA | |
| | TACTTAAAAAAGCACACACAACGTA | |
| | TGCTGCTCACTGCCTCAACAAACCC | |
| | TTTATTGCAAGGTACCCGGGTAGAG | |
| | AATCAATACACTGAGGAAGAAAGAC | |
| | TAGCAAAGTTCCTTCTGGACCGAGA | |
| | ATTGGTTATGCCACGTGTGGCACAT | |
| | ACAGTCTTTGAGACCACTGTTGCCG | |
| | GGAGACGAAAGCATCTGCAAGGGTT | |
| | AATTGACACTACACCGACTATTATT | |
| | AAATATGCCCTTCATCACCACCCTA | |
| | TTTCTTTCAAGAAAAGTATGCTGAT | |
| | ATCATCTTACTCAGCTGACTACATT | |
| | ATGTCGTTTATTGAGACTATCGCAA | |
| | CAGTGGAATACCCAAAGCGTGACAC | |
| | CATGCAGCTCTGGAACAGAGGACTA | |
| | ATTGGTGTCGACACTTGCGCGGTCA | |
| | CACTTGCGGATTACGCAAGAACATA | |
| | TTCGTGGTGGGAGATCCTGAAGGGT | |
| | AGGTCAATAAAGGGAGTTACCACAC | |
| | CTGATACATTAGAACTTTGCTCTGG | |
| | GAGCTTAATAGAGCAAGGCCATCCA | |
| | TGTTCTCAGTGCACAATGGGTGATG | |
| | AATCCTTTTCATGGTTCTTCCTCCC | |
| | AGGGAATATTGATATTGAAAGACCG | |
| | GACTTTTCTAGGGTGGCCCAGAGAA | |
| | TCGCTTATGTCGGCTCAAAAACGGA | |
| | AGAAAGGCGGGCAGCTTCGTTGACG | |
| | ACAATCAAAGGGATGTCAACTCACC | |
| | TTAGGGCGGCACTAAGAGGGGCGAG | |
| | TGTTTACATCTGGGCGTATGGAGAC | |
| | AGCGACAAAAATTGGGACGACGCTA | |
| | CAAAGCTTGCTAACACAAGATGTGT | |
| | AATATCTGAAGACCATCTGCGTGCC | |
| | CTTTGCCCAATCCCGAGTTCAGCAA | |
| | ACATACAGCATAGGCTGATGGATGG | |
| | GATAAGCGTAACGAAGTTCACTCCC | |
| | GCATCCCTAGCAAGAGTGTCATCGT | |
| | ATATTCATATTTCGAATGACCGGCA | |
| | TCAGAGTAGAATTGACGGTCAAGTG | |
| | ATCGAATCAAATGTGATTTTCCAAC | |
| | AAGTTATGCTTCTCGGTCTCGGTAT | |
| | TTTTGAGACATTTCACCCCTTGTCT | |
| | CACAGGTTTGTGACTAACCCCATGA | |
| | CACTCCACTTACACACAGGGTACTC | |
| | GTGTTGCATAAGGGAAGCTGATAAT | |
| | GGTGATTTCTTAGAATCCCCGGCTA | |
| | GTGTACCAGACATGACTATCACGAC | |
| | TGGTAATAAGTTCCTTTTTGACCCC | |
| | GTGCCCATTCAAGATGACGATGCTG | |
| | CAAAACTACAGGTATCTTCATTCAA | |
| | GTACTGTGAGATGGGCCTCGAAGTG | |
| | CTTGACCCACCAGGACTTGTAACCC | |
| | TACTATCTCTAGTGACTGCACGTAT | |
| | CTCTATTGATACATCTATAGGGGAG | |
| | AGTGCATACAACTCGATACACAATG | |
| | ATGCTATTGTCTCATTCGACAATTC | |
| | CATCAATTGGATATCTGAGTACACA | |
| | TACTGTGATCTTAGACTACTGGCAG | |
| | TAGCAATGGCTCGGGAGTTTTGTGA | |
| | CAACCTCTCTTATCAGCTTTACTAT | |
| | CTGAGGGTTAAAGGGCGACGGGCAA | |
| | TCCGGGATTATATCCGCCAAGCCCT | |
| | CTCGAGGATACCAGGGTTACAACTT | |
| | GCTAATATAGCCTTGACTATATCTC | |
| | ATCCGGGAATTTGGGCAAGACTGAG | |
| | GCTAATTGGGGCAGTAAGTGCTGGA | |
| | AATAGTCCCATCAGTGCAACCGTAA | |
| | ATTATCCTGCTGCTGTGTGTGAGCT | |
| | CATATTATGGGGTTACGAACAATAT | |
| | ACTGCACAACTACTAGATGGTTACG | |
| | AGTTAGAAATTATAGTCCCGAATTA | |
| | TAAGGATGATGACCTGAACAGGAAG | |
| | GTTGAACATATACTAGCAAGACGGG | |
| | CTTGCCTGCTGAGTCTGCTGTGTGA | |
| | GTATCCAGGAAAATACCCGAATATT | |
| | AAAGACCTTGAACCTATTGAGAAAT | |
| | GCACTGCTCTGTCTGACCTGAATAA | |
| | ATTGTGGATGGCGACAGATCACAGA | |
| | ACTCGGGAATGTTTTTCCGGGATAT | |
| | CTCAGATATTTGATTCCCCCAAATT | |
| | AAATCCGTTCATCACTAATCTTTAC | |
| | TTCTTGAGTAGAAAGCTGCTCAACG | |
| | CGATTATAAGCAGCACGGACTGTAG | |
| | GGCCTACGTTGAGAACCTTTATGAA | |
| | GATATCGACATTGAACTAACATCTC | |
| | TCACTGAGGTTTTGCCCTTAGGAGA | |
| | GGATGATCAAATGATCACTGGGCCT | |
| | CTGCGCTTTGACCTTGAACTAAAAG | |
| | AACTCACCCCGGATTTTACTATCAC | |
| | TTGGTGTTGTTTTGACTCTACAGCA | |
| | GCACTGATGTCACGGTGCATTAATC | |
| | ATGCCACAGAAGGCGCAGAGCGCTA | |
| | CATCCGAAGAACGGTTGGGACAGCT | |
| | TCAACATCTTGGTATAAAGCAGCAG | |
| | GAATATTAACTACACCTGGCTTTCT | |
| | CAACCTCCCTAAAGGCAATGGCTTA | |
| | TATCTAGCTGAGTCATCAGGGGCCA | |
| | TCATGACTGTGATGGAGCATCTTGT | |
| | CTGCTCTAATAAAATATGGTATAAC | |
| | ACCTTGTTTAGCAATGAGCTCAACC | |
| | CACCTCAGAGGAATTTTGGTCCCAA | |
| | CCCAATTCAATTTGAAGAAAGTATC | |
| | GTGGGTAAACATATTGCAGCCGGGA | |
| | TTCCTTGCAAGGCAGGACATGTGCA | |
| | AGAGTTTGAGGTACTTTGGAGAGAG | |
| | GTAGATGAAGAGACAGATCTGACCT | |
| | CCATGAGATGTGTGAATTTTATCAT | |
| | GTCGAAAGTTGAACAGCACTCGTGT | |
| | CATATTGTATGCTGTGACTTAGAAT | |
| | TGGCTATGGGGACTCCCTTAGAAGT | |
| | GGCCCAATCTGCATATACGCATATT | |
| | GTAACCCTCGCCTTGCATTGCCTAA | |
| | TGATTAGCGGAAAATTAGTACTAAA | |
| | GTTGTATTTCTCACAAAATGCCCTC | |
| | TTACACCATGTTCTCTCTTTATTGC | |
| | TTGTATTGCCATTCCATGTAACAAT | |
| | CCACACTAACGGTTATTGCTCTCAC | |
| | CGAGGCTCTGAAGGGTATATCATTG | |
| | CCACGAGAACAGGAGTTGCTCTGGG | |
| | TTCAAATGTGTCCCAAGTACTAGGT | |
| | GGTGTGACTGAGATGGTACGGAAAG | |
| | GTCAGACCCTTGTCCCTGTAAAGGT | |
| | ACTTACAGCGATCTCCAATGGGTTC | |
| | AGAACTGTGTCAAGCTCTTTAGGCA | |
| | GACTAAGGGGTGAGCTCTATTCGCC | |
| | ATCGTGTAGCATTCCGCAGTCAGCT | |
| | ACCGACATGTTCCTCATTCAACTTG | |
| | GAGGGAAGGTGCAGTCAGATTGGAA | |
| | TACGAACTCTCGAGGCTATAGAGTG | |
| | GGTGAGACTGATCTCGTATTACAGG | |
| | ACATTATATCAATATTGAGCACACT | |
| | ACTTAAAGAAATAATACACGTAAGG | |
| | GAATCCAGGGAGTCAGTGGACAGGG | |
| | TTCTGTTGCTCGGGGCATACAACCT | |
| | ACAGGTGTCTGGAAAAGTAAGAACA | |
| | ATGGCCGCGGCTGCAACAAGGAACA | |
| | TATTGCATCTACATATAGTTAGACT | |
| | TATTGGAGACTCAATGTCCAATGTA | |
| | AGGAGACTAGTACCTCTGCTAGATA | |
| | AGGGCTTTATAGTAATATCAGACAT | |
| | GTATAGTGTGAAAGATTTCTTGAGA | |
| | AAAACTGAGTCCCCTAAGTACTTCT | |
| | TAAACAAGCTAGGCAAGAGCGAGAT | |
| | TGCACAGCTATTTGAGATAGAGTCC | |
| | AAGATTATTCTGAGCAGGGCAGAGA | |
| | TCAAGAATATTTTGAAGACAATAGG | |
| | GATTGTGGCTAAACAGCACTCAGAG | |
| | TGATCTCTCCAACCTTGCACCATTT | |
| | GAATTCTGGACTGTGGACGCGCATG | |
| | CCTAAGCGCACCAACTTGCCGTGAC | |
| | GATTGATGTAATCCTTGATATGAAC | |
| | TACTAATCATTTGGAATTTATTTAC | |
| | TTCCCGAAATCACCCATAGACCGGA | |
| | ATCGATACCGGAGATTATTTTTTAA | |
| | TAAAAAACCTGGAAAGTCGACAAGG | |
| | ATCATAGTCAAAAAGCTTATGATTT | |
| | CCTTGTTTGGT | |
| Avian | ACCAAACAAGGACTGCATAAGCAGT | SEQ ID |
| paramyxovir | GTAAAACTTTTAATAAAAAATAACT | NO: 10 |
| us 7 | TTCGTGAGGGTGAATCGATCATCGC | |
| strain | TCGAAGCCGATATCGACTCACCCAA | |
| APMV-7/dove/ | ATTAGCTGCTTGTATAAGGATCCGA | |
| Tennessee/ | ATATCAATTGGAATCATGTCATCGA | |
| 4/75, | TTTTTACTGATTATACCAATTTGCA | |
| complete | AGAGCAATTAGTCAGACCGGTAGGC | |
| genome | CGGAAGGTTGATAATGCTTCAAGTG | |
| Genbank: | GCTTGTTGAAAGTTGAGATACCAGT | |
| FJ231524.1 | CTGCGTCCTGAATTCACAGGACCCA | |
| | GTTGAGAGACACCAGTTCGCAGTAT | |
| | TATGTACAAGGTGGATCTCAAGTTC | |
| | AATTGCCACAACTCCTGTCAAGCAA | |
| | GGTGCCCTGCTTTCTCTTCTCAGTT | |
| | TGCACACAGAAAACATGCGAGCGCA | |
| | TGTTCTATTAGCAGCCCGGTCAGGA | |
| | GATGCTAATATAACAATTCTAGAAG | |
| | TTGATCATGTAGATGTTGAAAAGGG | |
| | AGAATTACAATTTAATGCAAGGAGT | |
| | GGTGTCTCATCTGATAAAGCTGATC | |
| | GGCTGCTGGCTGTCGCAATGAATCT | |
| | TATTGCAGGTTGTCAGAATAACTCA | |
| | CCATTTGTCGACCCATCGATTGAGG | |
| | GTGATGAACCAACTGATATGACTGA | |
| | ATTTTTAGAGCTGGCTTATGGGTTA | |
| | GCGGTTCAAGCATGGGTAGCTGCAA | |
| | TAAAGAGTATGACGGCACCAGATAC | |
| | TGCTGCGGAGAGTGAGGGGCGGCGA | |
| | TTAGCAAAATACCAGCAGCAAGGTC | |
| | GTTTAACACGACGTGCTGCTCTTCA | |
| | AGCAACCGTGAGGGGGGAGTTGCAG | |
| | CGGATAATCAGGGGTTCTCTGGTAG | |
| | TTCGACACTTCCTTATAGGAGAAAT | |
| | CAGAAGAGCAGGAAGTATGGGAGAA | |
| | CAGACAACAGCCTATTATGCCATGG | |
| | TGGGAGATGTCAGCCAATACATAAA | |
| | GAATTCAGGAATGACTGCATTCTTC | |
| | CTGACATTACGATTTGGGGTGGGTA | |
| | CCAAGTATCCTCCCCTTGCAATGGC | |
| | TGCATTTTCAGGAGATCTCACTAAA | |
| | CTCCAGAGCCTGATCAGACTATATC | |
| | GAAATAAAGGTGACATAGGGCCTTA | |
| | TATGGCCCTACTCGAAGATCCTGAC | |
| | ATGGGCAACTTTGCTCCTGCAAATT | |
| | ACACCTTGCTCTATTCATATGCAAT | |
| | GGGCATTGGTTCTGTATTGGAGGCT | |
| | AGTATCGGTAGATACCAGTATGCGA | |
| | GAACATTCCTGAATGAATCATTCTT | |
| | TAGGTTGGGGGCCTCAACTGCTCAA | |
| | CAGCAACAAGGAGCACTGGATGAGA | |
| | AATTGGCTAACGAGATGGGGCTATC | |
| | AGACCAGGCAAGGGCAGCAGTTTCC | |
| | AGATTAGTTAATGAGATGGATATGG | |
| | ATCAGCAAGTAGCCCCCACACCAGT | |
| | TAATCCAGTCTTTGCAGGAGATCAA | |
| | GCAGCCCCACAGGCAAATCCTCCAG | |
| | CCCAACCAAGACAGAATGACACACC | |
| | ACAGCAGCCTGCTCCTCTTCAGCAG | |
| | CCAATTCGAATTGCCATGCCTCAAA | |
| | ATTATGATGATATGCCAGACTTAGA | |
| | GATGTAGACAGAACCCCAATCAAGC | |
| | AACAATTGGCATTAAGATCTAAGCT | |
| | GAATGTATGAGCACACGAGTACCCA | |
| | AGTATATTTGTTAGCAGTTGCATGA | |
| | AATCATTATCCATATTATTGATTTG | |
| | CAATATAGAAAATTACTGATAAACA | |
| | ATTAAGAATCATTTAATAAAAAAAT | |
| | TCCACAAAAATTAAAAAAATTGTGA | |
| | GGGGGAACACCTTTCAGTCGGTCAA | |
| | CTGCTGCTAATAACCTGCAATTATC | |
| | ACGTGGATTGAATATGGAATTCAGT | |
| | AATGATGCCGAGGTTGCCGCGCTCC | |
| | TGGATCTTGGAGATAGCATCATTCA | |
| | GGGCATTCAGCATGCAACAATGGCT | |
| | GATCCGGGAACACTAGGGAAGTCAG | |
| | CTATTCCTGCAGGTAATACCAAACG | |
| | CTTAGAGAAATTATGGGAGAAAGAA | |
| | TCTGTTCCTAATCATGATAATATGA | |
| | TTCACTCTTCCATGAGTGCAGAACC | |
| | TATAAGCGGGGAACTACCTGAGGAA | |
| | AACGCTAAAACTGAACCAACAGGGA | |
| | CTCAAGAAATGCCAGAACAAATTCA | |
| | AAAGAATGACAATCTCCAACCTGCA | |
| | TCCATCGATAACATATTGAGCAGCA | |
| | TTAATGCATTAGAGTCAAAACAGGT | |
| | TAAAAAAGGGTTAGTGCTATCGCCC | |
| | CAATCACTGAAAGGTGTGTCCCCCT | |
| | TAATCAAGAACCAGGATCTGAAGAA | |
| | CACCATGCAGGACCTGGAAACCAAA | |
| | CCCAAGGCTGTAACGACTGTAAATC | |
| | CATTAGCAAACCGACAAGTGTCACC | |
| | TGGAAGCCTGGTCATAGACGAGAGT | |
| | ATTCCTTTGCTTGGAGTGCAGGAAC | |
| | AAACAAATTTATTGTCTCCTCGTGG | |
| | TGTAACCCAACTTGCGCCCCAATCA | |
| | GACCCTATCCTACAGTCGAACGATG | |
| | CAGGTGCGGGAATTGCCCAAAATTC | |
| | TGCCCTGGATGTCAATCAGCTCTGG | |
| | GATGTAATCAATCAGCAACACAAGA | |
| | TGCTGATAAACCTACAAAATCAAGT | |
| | AACAAAGATCACTGAGCTGGTTGCT | |
| | TTAATTCCAATTCTTCGAAGTGATA | |
| | TTCAGGCTGTAAAGGGAAGTTGCGC | |
| | ATTATTAGAAGCACAGCTAGCATCT | |
| | ATAAGAATACTAGATCCTGGGAACA | |
| | TCGGGGTATCTTCATTAGATGATCT | |
| | TAAAACAGCAGGGAAACAAAGTGTA | |
| | GTTATTAATCAAGGGAGCTATACTG | |
| | ATGCAAAGGATCTGATGGTTGGGGG | |
| | AGGATTGATTCTTGATGAACTTGCT | |
| | AGACCTACTAAATTAGTCAATCCAA | |
| | AGCCACAACAATCTTCCAAAATATT | |
| | GGATCAGGCAGAAATTGAAAGTGTC | |
| | AAGGCCCTAATCCATACCTACACTC | |
| | ACGATGATAAGAAGCGGAACAAATT | |
| | CTTAACTGCACTTGACAAGGTGACA | |
| | ACCCAGGATCAGCTAACTCGCATCA | |
| | AGCAGCAAGTATTAAATCAATAGAT | |
| | AGACAATTAGCATTCATTCAAGCTA | |
| | TACTCATTTAAGTGCTTTGATTGTG | |
| | TTGCGGAAACTATATTGAGATAATT | |
| | TAGTCTTACATGCAAAATAACATTA | |
| | AAAATTAATTATGAGCAATCTTGAT | |
| | TTTTCTAACTCATAATCAACCTCCT | |
| | TCTCTATAAAGGCATACTTAGTATT | |
| | GCAAAAAGAGAAAATTAAGAAAAAA | |
| | AGAAAAAGAAAATTGAGGGAGACCG | |
| | CTTGATAGATCTGTGATCGGTCTCA | |
| | TAACCTCAAATTAAAATGGAATCTA | |
| | TATCTCTGGGGTTATATGTTGATGA | |
| | AAGTGATCCAGCATGCTCATTACTT | |
| | GCATTCCCCATAATCATGCAGACTA | |
| | CAAGTGAAGGAAAGAAGGTCTTACA | |
| | ACCGCAAGTCAGAATAAACCGTCTA | |
| | GGGAGTATATCGATAGAAGGAGTTC | |
| | GGGCAATGTTCATAAATACATATGG | |
| | CTTCATTGAGGAGAGGCCTACGGAA | |
| | AGGACAGGTTTCTTTCAGCCAGGCG | |
| | AAAAAAATCAGCAGCAAGTTGTGAC | |
| | AGCTGGTATGCTGACATTGGGCCAA | |
| | ATAAGGACCAATATAGACCCGGACG | |
| | AAATTGGAGAGGCATGCTTGAGACT | |
| | CAAAGTGAATGCTAAAAAATCAGCA | |
| | GCAAGTGAGGAGAAGATAGTATTTA | |
| | GCATTCTTGAAAAGCCTCCCGCCCT | |
| | GATGACTGCACCTGTAGTACAAGAT | |
| | GGGGGCTTAATTGCTAAAGCAGAAG | |
| | GATCAATCAAATGCCCAGGTAAGAT | |
| | GATGAGTGAAATTCACTACTCATTT | |
| | AGAGTAATGTTTGTGAGTATCACAA | |
| | TGCTGGATAATCAGAGCCTATACAG | |
| | AGTACCAACAGCCATCAGCTCGTTC | |
| | AAAAATAAAGCTCTATATTCTATTC | |
| | AGTTAGAGGTATTGCTGGAAGTTGA | |
| | TGTGAAGCCTGAGAGCCCCCAGTGT | |
| | AAATTTCTAGCAGACCAGAAAGGGA | |
| | AGAAAGTTGCTTCTGTATGGTTCCA | |
| | TCTCTGCAATTCTAAAAAGACGAAT | |
| | GCCAGCGGGAAACCGAGATCATTAG | |
| | AGGATATGAGAAAGAAGGTCCGAGA | |
| | TATGGGAATCAAAGTGTCTCTGGCC | |
| | GACCTTTGGGGCCCTACGATCATCG | |
| | TCAGGGCCACAGGGAAGATGAGTAA | |
| | ATATATGCTAGGATTTTTCTCTACC | |
| | TCAGGGACTTCATGTCATCCAGTAA | |
| | CAAAGAGTTCACCAGATTTGGCAAA | |
| | AATATTATGGTCATGCTCAAGCACA | |
| | ATCATCAAAGCAAATGCCATTGTTC | |
| | AAGGGTCAGTCAAAGTCGATGTCCT | |
| | GACCCTCGAAGATATCCAAGTTTCC | |
| | AGTGCTGCAAAAATCAACAAATCAG | |
| | GAATAGGGAAGTTTAATCCATTTAA | |
| | GAAATAAAGTCATATGCAGATTAAA | |
| | ATTTGATCAAGATTGGTCTTAGCAA | |
| | ATTAACTGAATGTAATTATAAAATA | |
| | CCTCAGTAAAATGCTAATGAATCAG | |
| | TGGATGATATTGAATTAGCAGATTG | |
| | AAAATTAAAGAAAACCTTATGAGGG | |
| | CGAATGAGCTTAGATGATTTAATAA | |
| | AGGAGACTAATCCAACATTTCCCTC | |
| | AAATTAACAAAATCAGAAAGTAAAA | |
| | AGAAAGGGAGCAATGAGAGTACGAC | |
| | CTTTAATAATAATCCTGGTGCTTTT | |
| | AGTGTTGCTGTGGTTAAATATTCTA | |
| | CCCGTAATTGGCTTAGACAATTCAA | |
| | AGATTGCACAAGCAGGTATTATCAG | |
| | TGCACAAGAATATGCAGTTAATGTG | |
| | TATTCACAGAGTAATGAGGCTTACA | |
| | TTGCACTGCGCACTGTGCCATATAT | |
| | ACCTCCACACAATCTCTCTTGTTTC | |
| | CAGGATTTAATCAACACATACAATA | |
| | CAACGATTCAAAACATATTCTCACC | |
| | AATTCAGGATCAAATCACATCTATA | |
| | ACATCGGCGTCAACGCTCCCCTCAT | |
| | CAAGATTTGCAGGATTAGTAGTCGG | |
| | TGCAATCGCTCTCGGAGTAGCGACA | |
| | TCTGCACAAATAACTGCAGCCGTGG | |
| | CACTCACAAAGGCACAGCAGAACGC | |
| | TCAAGAAATAATACGATTACGTGAT | |
| | TCTATCCAAAATACTATCAATGCTG | |
| | TGAATGACATAACAGTAGGGTTAAG | |
| | TTCAATAGGAGTAGCACTAAGCAAG | |
| | GTCCAAAACTACTTGAATGATGTGA | |
| | TAAACCCTGCTCTGCAGAACCTGAG | |
| | CTGCCAGGTTTCTGCATTAAACTTA | |
| | GGGATCCAATTAAATCTTTATTTAA | |
| | CCGAAATTACAACTATCTTTGGACC | |
| | GCAAATTACAAATCCATCATTGACC | |
| | CCATTGTCAATTCAGGCATTATACA | |
| | CCCTAGCAGGAGATAACCTGATGCA | |
| | ATTTCTTACCAGGTATGGCTATGGA | |
| | GAGACAAGTGTTAGCAGTATTCTCG | |
| | AGTCAGGACTAATATCAGCACAAAT | |
| | TGTATCTTTTGATAAACAGACAGGC | |
| | ATTGCAATATTGTATGTCACATTAC | |
| | CATCAATTGCGACTCTTTCCGGTTC | |
| | TAGAGTTACCAAATTGATGTCAGTT | |
| | AGTGTCCAAACTGGAGTTGGAGAGG | |
| | GTTCTGCTATTGTACCATCATACGT | |
| | TATTCAGCAGGGAACAGTAATAGAA | |
| | GAATTTATTCCTGACAGTTGCATCT | |
| | TCACAAGATCAGATGTTTATTGTAC | |
| | TCAATTGTACAGTAAATTATTGCCT | |
| | GATAGCATATTGCAATGCCTCCAGG | |
| | GATCAATGGCAGATTGCCAATTTAC | |
| | TCGCTCATTGGGTTCATTTGCAAAC | |
| | AGATTCATGACCGTTGCAGGTGGGG | |
| | TGATAGCAAATTGTCAGACAGTCCT | |
| | GTGCCGATGCTATAATCCAGTTATG | |
| | ATTATTCCCCAGAACAATGGAATTG | |
| | CTGTCACTCTGATAGATGGTAGTTT | |
| | ATGTAAAGAACTTGAATTGGAGGGG | |
| | ATAAGACTAACAATGGCAGACCCAG | |
| | TATTTGCTTCATACTCTCGTGATCT | |
| | GATTATAAATGGGAATCAATTTGCT | |
| | CCGTCTGATGCTTTAGACATTAGTA | |
| | GCGAATTAGGTCAACTGAATAACTC | |
| | AATTAGCTCAGCAACTGATAATTTA | |
| | CAGAAGGCACAGGAATCATTGAATA | |
| | AGAGTATCATTCCAGCTGCGACTTC | |
| | CAGCTGGTTAATTATATTACTATTT | |
| | GTATTAGTATCAATCTCATTAGTGA | |
| | TAGGATGTATCTCCATTTATTTTAT | |
| | ATATAAACATTCAACCACAAATAGA | |
| | TCACGAAATCTCTCAAGTGACATCA | |
| | TCAGTAATCCTTATATACAGAAAGC | |
| | TAATTGATGAATTAATTTCTAAAAA | |
| | ATAATTTGATGTTCTAATAGGAGAA | |
| | TGCAATATCAATATGTCCATTATAA | |
| | TATACTTGATTGATTGAAAGATCTG | |
| | ATAATAATAGTTTATAAGACACTAA | |
| | GTAAGAGTTAAATGCTAAAGCAAGT | |
| | TGATTCCTAAATTTCTGCACAATAG | |
| | GACCATACTATATCATATTAGATAA | |
| | TTAATAAAAAACGCCCTATCCTGAG | |
| | GGCGAAAGGCCGATCATTAGTGACT | |
| | TTAACCGTTGCTCTCCCAATTTAAA | |
| | ATATATTTCACATGGAGTCAATCGG | |
| | GAAAGGAACCTGGAGAACTGTGTAT | |
| | AGAGTCCTTACGATTCTATTAGATG | |
| | TAGTGATCATTATTCTCTCTGTGAT | |
| | TGCTCTGATTTCATTGGGTCTGAAG | |
| | CCAGGTGAGAGGATCATCAATGAAG | |
| | TCAATGGATCTATCCATAATCAACT | |
| | TGTTCCCTTATCGGGGATTACTTCC | |
| | GATATTCAGGCAAAAGTCAGCAGCA | |
| | TATATCGGAGCAACTTGCTAAGTAT | |
| | CCCACTACAACTTGATCAAATCAAC | |
| | CAGGCAATATCATCATCTGCTAGGC | |
| | AAATTGCTGATACAATCAACTCGTT | |
| | TCTCGCTCTGAATGGCAGTGGAACT | |
| | TTTATTTATACAAATTCACCTGAGT | |
| | TTGCAAATGGTTTCAATAGAGCAAT | |
| | GTTCCCAACCCTAAATCAAAGCTTA | |
| | AATATGCTAACACCTGGTAATCTAA | |
| | TTGAATTTACTAATTTTATTCCAAC | |
| | TCCAACAACAAAATCAGGATGTATC | |
| | AGAATACCATCATTTTCAATGTCAT | |
| | CAAGTCACTGGTGTTATACCCATAA | |
| | TATCATTGCTAGTGGATGTCAGGAT | |
| | CATTCAACCAGTAGTGAATACATAT | |
| | CGATGGGGGTTGTTGAAGTGACTGA | |
| | TCAGGCTTACCCGAACTTTCGGACA | |
| | ACTCTTTCTATTACATTAGCTGATA | |
| | ATCTAAACAGAAAGTCATGTAGCAT | |
| | TGCAGCAACTGGGTTCGGGTGTGAT | |
| | ATATTATGTAGTGTTGTCACTGAGA | |
| | CAGAAAATGATGATTATCAATCACC | |
| | AGAACCGACTCAGATGATCTATGGA | |
| | AGATTATTTTTTAATGGCACATATT | |
| | CAGAGATGTCATTGAATGTGAACCA | |
| | AATGTTCGCAGATTGGGTTGCAAAT | |
| | TATCCAGCAGTTGGATCAGGAGTAG | |
| | AGTTAGCAGATTTTGTCATTTTCCC | |
| | ACTCTATGGAGGTGTTAAAATCACT | |
| | TCAACCCTAGGAGCATCTTTAAGCC | |
| | AGTATTACTATATTCCCAAGGTGCC | |
| | CACAGTCAATTGCTCTGAGACAGAT | |
| | GCACAACAAATAGAGAAGGCAAAAG | |
| | CATCCTATTCACCACCTAAAGTGGC | |
| | TCCAAATATCTGGGCTCAGGCAGTC | |
| | GTTAGGTGCAATAAATCTGTTAATC | |
| | TTGCAAATTCATGTGAAATTCTGAC | |
| | ATTTAACACTAGCACTATGATGATG | |
| | GGTGCTGAGGGAAGACTCTTGATGA | |
| | TAGGAAAGAATGTATACTTTTATCA | |
| | ACGATCTAGTTCGTATTGGCCAGTG | |
| | GGAATTATATATAAATTAGATCTAC | |
| | AAGAATTGACAACATTTTCATCAAA | |
| | TCAATTGCTGTCAACAATACCAATT | |
| | CCATTTGAGAAATTCCCTAGACCTG | |
| | CATCTACTGCTGGTGTATGTTCAAA | |
| | ACCAAATGTGTGTCCTGCAGTATGC | |
| | CAGACTGGTGTTTATCAAGATCTCT | |
| | GGGTACTATATGATCTTGGCAAATT | |
| | AGAAAATACCACAGCAGTAGGATTG | |
| | TATCTAAACTCAGCAGTAGGCCGAA | |
| | TGAACCCTTTTATTGGGATTGCAAA | |
| | TACGCTATCTTGGTATAATACAACT | |
| | AGATTATTCGCACAGGGTACTCCAG | |
| | CATCATATTCAACAACGACCTGCTT | |
| | CAAAAATACTAAGATTGACACGGCA | |
| | TACTGCTTATCAATATTAGAATTAA | |
| | GTGATTCTTTGTTAGGATCATGGAG | |
| | AATTACACCATTATTGTACAATATC | |
| | ACTTTAAGTATTATGAGCTAGATCC | |
| | TGTTTTAACATTGAATCGTATGAAC | |
| | TTATAAGACTGAAGGATGTCTGTTG | |
| | GTATTAAGCATCATAAAACACGGTT | |
| | GTTTTTGATTTGACACCTAATCGTA | |
| | CTCAATACTCTCCATAGATTTAATC | |
| | TAACAGATTTAGATACTATTGATCA | |
| | TATAGGCATAGATGGTATATGGGCA | |
| | ATTAGATTGAACTGAGTTAAATCCG | |
| | ATTGATACTTATCAAATTAAGATCT | |
| | AGATTATTTAATAAAAAATCTAAGT | |
| | TAGAAAATGAGGGGGACCTCATTAT | |
| | GGAGTTCAGACAATCTGATCAAATA | |
| | ATACATCCTGAAGTGCATCTAGATT | |
| | CACCTATTATTGGGAATAAAATACT | |
| | CTATTTATGGCGAATTACAGGCTTA | |
| | CCTACTCCGCCTGTTCTTGAGCTTA | |
| | ACTCTACTATATCGCCTGAAGTCTG | |
| | GACAAACTTGAAAGCCAATGATCCT | |
| | AGAGTAGCCTTTAAATGGGACAAAC | |
| | TAAGACCACGGTTGCTAACATGGGC | |
| | AGCACATCAAGGGATATCACTATCG | |
| | GATCTGATCCCTATTACACATCCTG | |
| | AGTCATTGCAGTGGTTAACAACAAT | |
| | ATCCTGTCCTAAAATTGATGAAAAT | |
| | TTTGCGTTAATTAAGAAGTGCCTTC | |
| | TTAGAACAAGGGACTATACAGCATC | |
| | AGGATTTAAGAATTTATTCCAAATG | |
| | ATCTCACAGAAATTGACGTCGACGA | |
| | ATATTCTATTTTGCGCAGAAAATCC | |
| | GACAACTCCCCCCATCTCCGACGAA | |
| | GCATCCTGGGCATTAAAGAATCCTG | |
| | AGCACTGGTTTAATACACCTTGGTC | |
| | ATCTTGTTGTATGTTTTGGTTACAT | |
| | GTGAAACAGACTATGAGGAACTTAA | |
| | TTAGAATACAACGATCTCAACCAGA | |
| | ATCACAAAGCATATACAGTATCACG | |
| | GTTGATAACTTGTTTGTTGGATTGA | |
| | CTCCTGACTTGTGTGTCATAGCTGA | |
| | TTCTCAAAGACAATCAATTACAGTA | |
| | CTGTCATTTGAGTGTGTATTGATGT | |
| | ATTGTGACTTAATTGAAGGTCGTAA | |
| | CAATGTTTATGACCTCTGTCAATTG | |
| | TCTCCTGTGCTAAGTCCTCTTCAAG | |
| | ATAGAATTTTACTTTTACTGAGATT | |
| | AATTGATTCTTTAGCATATGACATC | |
| | GGAGCGCCAATTTTTGATGTAATTG | |
| | CTTCTCTTGAATCTTTAGCATATGG | |
| | AGCTATTCAGCTATATGATTACGAC | |
| | ACAGAGGCAGCCGGTGATTTTTTCT | |
| | CATTTAATTTAAGAGAAATTTCCCA | |
| | GGTCATAGAAGAGAGCAAATGTAGG | |
| | AATCAAACCCATACTATAATCAGTG | |
| | CAATTAGTAAGATTTACACAGGGAT | |
| | CAATCCTGATCAAGCAGCTGAAATG | |
| | CTGTGTATCATGAGACTGTGGGGTC | |
| | ACCCATTGCTTTATGCATCCAAGGC | |
| | TGCATCTAAGGTTCGCGAGTCAATG | |
| | TGTGCACCTAAAGTTATCCAATTTG | |
| | ATGCAATGCTGCTTGTATTAGCATT | |
| | CTTTAAGAGAAGCATCATAAATGGA | |
| | TATAGACGAAAGCATGGTGGGCTAT | |
| | GGCCGAACATCATAGTTGAGTCACT | |
| | TCTTTCTGCAGAACTTGTCGCGGCA | |
| | CATCATGATGCAGTTGAATTGACAG | |
| | ACACTTTTGTTATTAAACACTATAG | |
| | AGAAGTAGCCATGATTGACTTCAAA | |
| | AAATCATTCGACTACGATATAGGGG | |
| | ATGACTTAAGTTTATACCTCAAGGA | |
| | TAAAGCAATTTGTCGACAGAAATCA | |
| | GAGTGGCTTAATATCTTCAAGGGTC | |
| | AATTGCTTGAGCCCGCTGTACGATC | |
| | GAAGCGAATTCGTGGAATAGGTGAA | |
| | AACCGATTACTGTTACATTTCTTGA | |
| | ATTCAGTCGATTTTGATCCTGAACA | |
| | AGAATTCAAATACGTCACTGATATG | |
| | GAGTACCTCTACGATGAAACATTCT | |
| | GTGCATCCTATTCACTGAAGGAAAA | |
| | AGAAGTGAAAAGAGATGGAAGAATA | |
| | TTCGCAAAAATGACACCAAAAATGA | |
| | GAAGCTGTCAAGTTTTATTAGAGGC | |
| | ATTGTTAGCAAAACATGTAAGCGAA | |
| | CTTTTCAAGGAGAATGGAGTCTCAA | |
| | TGGAGCAGATATCCCTCACAAAGTC | |
| | ATTGGTAGCCATGTCACAATTAGCT | |
| | CCCCGAGTGAATATGAGAGGTGGGA | |
| | GAGCAGCTAGATCAACAGACGTTAA | |
| | AATCAATCAACGAAGGGTCAAGTCA | |
| | ATCAAAGAGCATGTTAAATCGAGAA | |
| | ATGATTCGAATCAAGAGAAAATTGT | |
| | AATTGCAGGTTATCTGACTACTGAT | |
| | TTACAAAAATACTGCCTCAATTGGA | |
| | GATATGAATCAATAAAATTATTTGC | |
| | AAGAGCACTTAACCAATTATTTGGA | |
| | ATACCCCATGGATTTGAATGGATAC | |
| | ACTTAAGGCTCATAAGAAGTACAAT | |
| | GTTTGTTGGGGATCCTTACAATCCT | |
| | CCTGCATCAATCCAATCTTTGGATC | |
| | TCGATGAACAGCCTAATGATGATAT | |
| | TTTTATTGTCTCGCCACGTGGTGGG | |
| | ATTGAAGGATTATGTCAGAAGATGT | |
| | GGACACTCATCTCAATTGCATTAAT | |
| | TCAAGCTGCAGCTGCAAAAATAGGA | |
| | TGTCGGGTTACAAGTATGGTACAGG | |
| | GAGATAATCAGGTTATTGCTATCAC | |
| | CAGAGAAGTGCGAGTGGGGGAACCT | |
| | GTGAGGGAGGCGTCACGAGAACTCA | |
| | GATTATTGTGTGATGAGTTCTTCAC | |
| | TGAATTCAAACAATTAAACTACGGA | |
| | ATAGGGCACAATCTTAAAGCAAAAG | |
| | AAACTATCAAGAGTCAATCGTTTTT | |
| | TGTATATAGCAAGAGAGTTTTCTTT | |
| | GAGGGAAGAGTGTTAAGTCAGATAT | |
| | TGAAGAATGCCTCAAAATTGAATCT | |
| | AATTTCTGACTGTCTGGCTGAAAAT | |
| | ACAGTTGCTTCATGTAGCAATATTT | |
| | CTTCTACTGTAGCAAGGCTAATAGA | |
| | GAATGGCCTTGGGAAAGACGTAGCC | |
| | TTCATTTTAAACTTTCAGACTATTA | |
| | TAAGGCAACTGATTTTTGATGAAGT | |
| | ATATACGATTTCATTGAACTATAGT | |
| | ACAGCAAGACGGCAGGTGGGAAGCG | |
| | AGAATCCTCACGCATTGGCTATAGC | |
| | CGCTTTGATTCCTGGTCAACTTGGG | |
| | GGATTCAATTTCCTAAACGTTGCTA | |
| | GGTTATTTACACGGAATATCGGGGA | |
| | TCCAATCACTTGCTCATTGAGTGAT | |
| | ATCAAATGGTTTGCAAAAGTTGGAT | |
| | TGATGCCTGAGTACATCCTTAAAAA | |
| | CATTGTTTTGAGGGCACCAGGTTCA | |
| | GGAACATGGACAACTTTAGTCGCTG | |
| | ATCCCTACTCCTTAAACATTACGTA | |
| | CACAAAATTGCCTACGTCGTACCTA | |
| | AAGAAACATACACAGAGGACATTAG | |
| | TTGCTGATTCCCCTAATCCGTTGCT | |
| | TCAGGGGGTGTTTCTATTAAATCAG | |
| | CAGCAGGAGGATGAAGCATTATGTA | |
| | AATTTCTTCTTGACCGAGAACAAGT | |
| | GATGCCACGAGCTGCCCATGTAATC | |
| | TATGATCAGTCAGTTCTCGGCCGGA | |
| | GGAAATATTTACAAGGGCTTGTTGA | |
| | TACTACACAGACAATCATAAGGTAT | |
| | GCACTCCAAAAAATGCCGGTATCAT | |
| | ACAAAAAGAGTGAAAAAATCCAAAA | |
| | TTACAATCTCCTCTACATACAATCA | |
| | CTTTTTGATGAGGTCTTGACACAGA | |
| | ATGTCATTCATAGTGGATTGGATAC | |
| | TATATGGAAAAGAGATCTAATTAGC | |
| | ATTGAGACCTGTTCTGTCACACTTG | |
| | CCAATTTTACGAGGACTTGCTCGTG | |
| | GTCTAATATTCTACAGGGCAGGCAA | |
| | ATTGTTGGAGTTACAACTCCAGACA | |
| | CGATAGAATTGTGTACCGGTTCTTT | |
| | GATTTCTTGCAACAGTGCATGTGAG | |
| | TTTTGTAGAATTGGAGATAAAAGCT | |
| | ACTCTTGGTTTCATACACCAGGGGG | |
| | TATCTCATTTGATACAATGAGCCCT | |
| | GGCAATCTGATTCAAAGAGTGCCGT | |
| | ACCTAGGATCAAAGACTGATGAACA | |
| | GCGAGCTGCCTCTCTAACAACCATC | |
| | AAGGGGATGGATTACCATCTGAGAC | |
| | AAGCTCTTCGAGGAGCATCATTGTA | |
| | TGTGTGGGCATATGGAGAGACTGAT | |
| | CAGAATTGGTTAGATGCGCTGAAGT | |
| | TAGCAAACACCCGGTGCAATGTAAC | |
| | ATTACAAGCTTTGACTGCACTCTGC | |
| | CCAATACCGAGTACCGCAAATCTAC | |
| | AACACCGGCTTGCGGATGGAATAAG | |
| | TACAGTTAAATTCACACCTGCAAGT | |
| | TTGTCACGAATAGCAGCTTATATTC | |
| | ACATTTGTAATGACCAACAAAAGCA | |
| | TGATAACCTAGGGAATAGTTTTGAA | |
| | TCAAATCTGATTTACCAGCAAATAA | |
| | TGCTTCTTGGAACAGGAATATTTGA | |
| | AACAATTTTCCCACTATCAGTTCAA | |
| | TATATCCACGAGGAACAAACACTTC | |
| | ACTTGCACACTGGATTTTCCTGTTG | |
| | TGTCAGGGAAGCTGACACAATGATT | |
| | ATAGATGAGAGCAGAACTGGATTCC | |
| | CAGGATTGACAGTGACTAAGAGTAA | |
| | TAAGTTTTTATTCAACCCTGACCCT | |
| | ATTCCTGCAGTGTGGGCAGATAAAA | |
| | TATTCACGACTGAATTTAGATTCTT | |
| | CGAGTACAATATAGAGAATCAAGGA | |
| | ACTTATGAACTAATAAAATTTCTTT | |
| | CTTCTTGCTGCGCGAAAGTTGTTAC | |
| | AGAATCGCTAGTTCAGGATACTTTC | |
| | CATAGTTCTGTCAAAAATGATGCAA | |
| | TAATTGCGTATGACAATTCAATTAA | |
| | TTACATCAGTGAGCTACAACAATGT | |
| | GACATTGTTCTGTTTAGCAGTGAAC | |
| | TTGGAAAGGAATTACTTCTAGATTT | |
| | AGCTTACCAGCTGTACTACCTTCGA | |
| | ATTAGATCGAAACGAGGTATAATTA | |
| | GTTACTTGAAGGTACTGCTGACTCG | |
| | GCTTCCAATTATTCAGTTTGCACCG | |
| | CTTGCGTTGACAATATCACATCCTG | |
| | TAATCTACGAGCGATTACGCCAACG | |
| | GAGGTTGGTTATGGAACCGTTGCAA | |
| | CCTTATTTGGCTTCGATAGATTATG | |
| | TCAAAGCCGCAAGAGAGCTTGTTTT | |
| | GATTGGTGCTTCTTCTTACCTCTCA | |
| | ATGCTTGAGACAGGTTTAGATACCA | |
| | CTTACAACATATACAGTCATTTAGA | |
| | CGGGGATTCAGAGGGCAAGATTGAT | |
| | CAGGCGATGGCAAGGAGACTGTGCC | |
| | TAATCACATTATTAGTGAATCCTGG | |
| | ATATGCATTACCTGTGATCAAAGGA | |
| | CTAACTGCAATTGAGAAATGTAGAC | |
| | TATTAACAGATTTTTTACAATCAGA | |
| | TATCATTTCTGTTTCTTTATCTGAG | |
| | CAGATTGCAACACTTATTCTAACAC | |
| | CAAAGATTGAAGTGCACCCGACAAA | |
| | TTTATACTATATGATGCGGAAGACC | |
| | TTGAATCTAATCCGGTCACGAGATG | |
| | ATACAGTTGTGATCATGGCAGAATT | |
| | GTATAATATAGATCAAGAGTCTGCG | |
| | ATAATGAGGGTTGAATCAGAAGAGG | |
| | ACGGCCCTGTAGACAAAATGAATCT | |
| | TGCACCCATACTAAGGCTTGTGCCA | |
| | ATCACATTCAAATCAATGGACTTGC | |
| | ATGCCTTAACTGGGCTAGGTAGAAA | |
| | AGAGGTGGAACTGATGGGTAGCCCA | |
| | GTTTGCAAAATCACTCAGAGATTAG | |
| | ATAAGTACATCTATCGCACAATTGG | |
| | CACCATATCTACTGCATGGTATAAA | |
| | GCAAGTAGTTTAATCGCCAGTGACA | |
| | TACTTAAGGGGGGCCCATTGGGGGA | |
| | CAGCTTATATTTATGTGAGGGAAGT | |
| | GGTAGTAGTATGACATGTTTGGAAT | |
| | ATTGTTTCCCTTCGAAAACAATCTG | |
| | GTATAATTCATTCTTCTCAAATGAG | |
| | CTAAATCCACCTCAACGGAACATCG | |
| | GCCCATTACCAACACAATTTTGTTC | |
| | AAGCATTGTCTATCACAATTTGAAT | |
| | GCTGAAGTCCCGTGCTCTGCAGGGT | |
| | TTATCCAAGATTTCAAAGTACTCTG | |
| | GGCCGACAAATCAGTGGAGACTGAT | |
| | ATTTCTACAACTGAATGTGTGAATT | |
| | TCATCCTAAGCAAAGTTGAACTTGA | |
| | AACATGCAAATTGATACATGCAGAC | |
| | CTTGATCTACCTATTGAGACCCCAA | |
| | GATCTGTCTGGATGGCTTGTGTCAC | |
| | AAATACATTCATTTTGGGAAATGCC | |
| | TTATTGAAGTCAGGAGGGAAATTGG | |
| | TCATGAAATTATATGCAGTAGATGA | |
| | GCTCCTCTTTTCATCTTGCTTAGGA | |
| | TTCGCATGGTGCCTTATGGACGATA | |
| | TAAATATCCTCCGAAATGGCTACTT | |
| | CAATGACAAATCAAAGGAATGCTAC | |
| | CTCATTGGGACAAAAAAGGTGACAA | |
| | TCCCGCACCAGAAAATCCAGGATAT | |
| | CCAGCAGCAAATAAATAAGATTGCT | |
| | AGTCAAGGGTTAAGTGTCATACCTG | |
| | AAGCTGTAATTCATGACATTTACAA | |
| | CCAGCTTGAGGACAGTATTAGATGT | |
| | GAGAAAAAATTCAAAAATGATAATG | |
| | CACCGACTTGGTCCAATGGGATCCT | |
| | CAATTCGACAGATCTATTACTAATA | |
| | AGACTTGGAGGGAAACCAATTGGGG | |
| | AATCACTATTAGAGTTAACATCCAT | |
| | ACAAGGCATGGATTATGATGATTTA | |
| | ACAGGGGATATAATTCAAGTAATAG | |
| | ACACAGCGCTAAATGAGATTATTCA | |
| | CCTCAAGTCTGATACTTCGAGCTTA | |
| | GATCTTGTACTGCTAATGTCTCCTT | |
| | ACAATCTGGCACTTGGAGGGAAAAT | |
| | AAGCACAATTCTGAAATCTGTTGTT | |
| | CACCAGACTCTAATACTCAGGATTA | |
| | TCCAATCTAGGCAGAATAAGGATAT | |
| | ACCATTAAAAGGATGGTTGTCTCTG | |
| | TTGAATCAAGGAGTCATCTCACTAT | |
| | CTTCATTGATCCCGTTGCATGATTA | |
| | TCTGAGGAAGAGTAAGTTGAGAAAA | |
| | TTTATAGTTCAAAAATTAGGCCAAC | |
| | AGGAATTACAAGCATTTTGGCAGAG | |
| | CAGGTCTCAACAAATGCTGAGTAGA | |
| | AGTGAGACCAAGTTGCTAATAAAAG | |
| | TGCTGAGTGCTGCTTGGAAGGGATT | |
| | GTTGTAAAATTGTAAATATACACTG | |
| | CATGTATATAAATTGGTTGCTACCC | |
| | TTATCAGCTAACCACAGGTGTAAAT | |
| | TTTCATATGGAATGCATATCAATAA | |
| | AGATAGGCATTTAAATTATACAATG | |
| | ATAACATATTTTAGGTTGACAACAA | |
| | TCATTGATATAATCACCAATAGTAG | |
| | CTCTATTACTTATTTGTTAATAATA | |
| | AATGGTACACTTTGAATTTAAGAAA | |
| | AAATTAGAATTGCTATATTTTATCG | |
| | CTATAGTGGGCCTGTCGGCTGCGTT | |
| | AGCGGTAAGACAAAGAGGACTTGTC | |
| | TTTTAAAAATTTATTAAAAAATCAT | |
| | TAATTGATCATATTGCTTTCCTTGT | |
| | TTGGT | |
| Avian | ACCAAACAAGGAATGCAAGACCAAC | SEQ ID |
| paramyxovir | GGGAACTTTAAATAAAACAATCGAA | NO: 11 |
| us 8 isolate | TCATTGGGGGCGAAGCAAGTGGATC | |
| APMV- | TCGGGCTCGAGGCCGAAACACTGGA | |
| 8/Goose/ | TTTCGCTGGAGGTTTTGAATAGGTC | |
| Delaware/ | GCTATAAGACTCAATATGTCATCTG | |
| 1053/76, | TATTCAATGAATATCAGGCACTTCA | |
| complete | AGAACAACTTGTAAAGCCGGCTGTC | |
| genome | AGGAGACCTGATGTTGCCTCAACAG | |
| Genbank: | GTTTACTCAGGGCGGAAATACCTGT | |
| FJ619036.1 | CTGTGTTACATTGTCTCAAGACCCC | |
| | GGTGAGAGATGGAGCCTTGCTTGCC | |
| | TTAATATCCGATGGCTTGTGAGTGA | |
| | TTCATCAACCACACCAATGAAGCAG | |
| | GGAGCAATATTGTCACTGCTGAGTC | |
| | TACATTCAGACAATATGCGAGCTCA | |
| | CGCAACATTAGCAGCAAGGTCTGCA | |
| | GATGCTTCACTCACCATACTTGAGG | |
| | TAGATGAAGTAGATATTGGCAACTC | |
| | CCTAATCAAATTCAACGCTAGAAGT | |
| | GGTGTATCTGATAAACGATCAAATC | |
| | AATTGCTTGCAATTGCGGATGACAT | |
| | CCCCAAAAGTTGCAGTAATGGGCAT | |
| | CCATTTCTTGACACAGACATTGAGA | |
| | CCAGAGACCCGCTCGATCTATCAGA | |
| | GACCATAGACCGCCTGCAGGGTATT | |
| | GCAGCTCAGATATGGGTGTCAGCCA | |
| | TAAAGAGCATGACAGCGCCTGACAC | |
| | CGCATCAGAGTCAGAAAGTAAGAGG | |
| | CTGGCCAAATACCAACAACAAGGCC | |
| | GACTGGTTAAGCAAGTACTTTTGCA | |
| | TTCTGTAGTCAGGACAGAATTTATG | |
| | AGAGTTATTCGGGGCAGCTTGGTAC | |
| | TGCGCCAGTTTATGGTTAGCGAGTG | |
| | CAAGAGGGCTTCAGCCATGGGCGGA | |
| | GACACATCTAGGTACTATGCTATGG | |
| | TGGGTGACATCAGTCTGTACATCAA | |
| | GAATGCAGGATTGACTGCATTTTTC | |
| | CTCACCCTGAAGTTCGGGGTTGGTA | |
| | CCCAGTATCCAACCTTAGCAATGAG | |
| | TGTTTTCTCCAGTGACCTTAAAAGA | |
| | CTTGCTGCACTCATCAGGCTGTACA | |
| | AAACCAAGGGAGACAATGCACCATA | |
| | CATGGCATTCCTGGAGGACTCCGAT | |
| | ATGGGAAATTTTGCTCCAGCAAATT | |
| | ATAGCACAATGTACTCTTATGCCAT | |
| | GGGCATTGGGACGATTCTGGAAGCA | |
| | TCTGTATCTCGATACCAGTATGCTA | |
| | GAGACTTTACCAGTGAGAATTATTT | |
| | CCGTCTTGGAGTTGAGACAGCCCAA | |
| | AGCCAGCAGGGAGCGTTTGACGAGA | |
| | GAACAGCCCGAGAGATGGGCTTGAC | |
| | TGAGGAATCCAAACAGCAGGTTAGA | |
| | TCACTGCTAATGTCAGTAGACATGG | |
| | GTCCCAGTTCAGTTCGCGAGCCATC | |
| | CCGCCCTGCATTCATCAGTCAAGAA | |
| | GAAAATAGGCAGCCTGCCCAGAATT | |
| | CTTCAGATACTCAGGGTCAGACCAA | |
| | GCCAGTCCCGAATCAACCCGCACCA | |
| | AGGGCCGACCCAGATGACATTGATC | |
| | CATACGAGAACGGGCTAGAATGGTA | |
| | ATTCAATCACCTCGACACATCCACC | |
| | TATACACCAATTCTGTGACATATTA | |
| | ACCTAATCAAACATTTCATAAACTA | |
| | TAGTAGTCATTGATTTAAGAAAAAA | |
| | TTGGGGGCGACCTCAACTGTGAAAC | |
| | ACGCCAGATCTGTCCACAACACCAC | |
| | TCAACAACCCACACAAGATGGACTT | |
| | CGCCAATGATGAAGAAATTGCAGAA | |
| | CTTCTGAACCTCAGCACCACTGTAA | |
| | TCAAGGAGATTCAGAAATCTGAACT | |
| | CAAGCCTCCCCAAACCACTGGGCGA | |
| | CCACCTGTCAGTCAAGGGAACACAA | |
| | GAAATCTAACTGATCTATGGGAAAA | |
| | GGAGACTGCAAGTCAGAACAAGACA | |
| | TCGGCTCAATCTCCACAAACCACAC | |
| | AAGTTCAGTCTGATGGAAATGAGGA | |
| | GGAAGAAATCAAATCAGAGTCAATT | |
| | GATGGCCACATCAGTGGAACTGTTA | |
| | ATCAATTAGAGCAAGTCCCAGAACA | |
| | AAACCAGAGCAGATCTTCACCAGGT | |
| | GATGATCTCGACAGAGCTCTCAACA | |
| | AGCTTGAAGGGAGAATCAACTCAAT | |
| | CAGCTCAATGGATAAAGAAATTAAA | |
| | AAGGGCCCTCGCATCCAGAATCTCC | |
| | CTGGGTCCCAAGCAGCAACTCAACA | |
| | GGCGACCCACCCATTGGCAGGGGAC | |
| | ACCCCGAACATGCAGGCACGGACAA | |
| | AACCCCTGACCAAGCCACATCAAGA | |
| | GGCAATCAATCCTGGCAACCAGGAC | |
| | ACAGGAGAGAATATTCATTTACCAC | |
| | CTTCCATGGCACCACCAGAGTCATT | |
| | AGTTGGTGCAATCCGCAATGTACCC | |
| | CAATTCGTGCCAGACCAATCTATGA | |
| | CGAATGTAGATGCGGGGAGTGTCCA | |
| | ACTACATGCATCATGTGCAGAGATG | |
| | ATAAGTAGAATGCTTGTAGAAGTTA | |
| | TATCTAAGCTTGATAAACTCGAGTC | |
| | GAGACTGAATGATATAGCAAAAGTT | |
| | GTAAACACCACCCCCCTTATCAGGA | |
| | ATGATATTAACCAACTTAAGGCCAC | |
| | AACTGCACTGATGTCCAACCAAATT | |
| | GCTTCCATACAAATTCTTGACCCAG | |
| | GGAATGCAGGGGTGAGGTCCCTCTC | |
| | TGAAATGAGATCTGTGACGAAGAAA | |
| | GCTGCTGTTGTAATTGCAGGATTTG | |
| | GAGACGACCCAACTCAAATTATTGA | |
| | AGAAGGTATCATGGCCAAAGATGCT | |
| | CTTGGAAAACCTGTGCCTCCAACAT | |
| | CTGTTATCGCAGCCAAAGCTCAGAC | |
| | TTCTTCCGGTGTGAGTAAGGGTGAA | |
| | ATAGAAGGATTGATTGCATTGGTGG | |
| | AAACATTAGTTGACAATGACAAGAA | |
| | GGCAGCGAAACTGATTAAAATGATT | |
| | GATCAAGTTAAATCCCACGCCGATT | |
| | ACGCCCGAGTCAAGCAGGCAATATA | |
| | TAATGCATAATATTGTAATTATACA | |
| | AACAATCAATACTGCTGTCGGTTGC | |
| | ACCCACCTTAGCAAATCAATAATCT | |
| | TTTAAAATTGATTGATTAAGAAAAA | |
| | ATTGACTACAATAAGGAAAGAACAC | |
| | CAAGTTGGGGGCGAAGTCACGATTG | |
| | ACCACAGTCGCTATCTGTAAGGCTC | |
| | CTCACCAAAAATGGCATATACAACA | |
| | CTAAAACTGTGGGTGGATGAGGGTG | |
| | ACATGTCGTCTTCGCTTCTATCATT | |
| | CCCGTTGGTACTAAAAGAGACAGAC | |
| | AGAGGCACAAAGAAGCTTCAACCAC | |
| | AGGTAAGGGTAGATTCAATTGGCGA | |
| | TGTGCAGAATGCCAAAGAGTCCTCG | |
| | ATATTCGTGACTCTATATGGTTTCA | |
| | TCCAAGCAATTAAGGAGAATTCAGA | |
| | TCGATCGAAATTCTTCCATCCAAAA | |
| | GATGACTTCAAACCTGAGACAGTCA | |
| | CTGCAGGACTGGTAGTAGTGGGTGC | |
| | AATCCGAATGATGGCTGATGTCAAT | |
| | ACCATCTCTAATGATGCACTAGCGC | |
| | TGGAGATCACTGTTAAGAAATCTGC | |
| | AACTTCTCAAGAGAAAATGACGGTG | |
| | ATGTTCCACAATAGCCCCCCTTCAT | |
| | TGAGAACTGCAATAACTATCCGAGC | |
| | AGGAGGTTTCATCTCGAATGCAGAC | |
| | GAAAATATAAAATGTGCCAGCAAGT | |
| | TGACTGCAGGAGTGCAGTACATATT | |
| | CCGTCCAATGTTTGTTTCAATCACT | |
| | AAATTACACAATGGCAAACTATATA | |
| | GGGTGCCCAAAAGTATCCACAGCAT | |
| | CTCGTCTACCCTACTGTATAGTGTG | |
| | ATGTTGGAGGTAGGATTCAAAGTGG | |
| | ACATCGGGAAGGATCATCCCCAGGC | |
| | AAAAATGCTGAAGAGGGTCACAATT | |
| | GGCGATGCAGACACATACTGGGGAT | |
| | TTGCATGGTTCCACCTGTGCAATTT | |
| | CAAAAAGACATCCTCTAAGGGAAAG | |
| | CCGAGAACGCTAGACGAACTGAGGA | |
| | CAAAAGTCAAAAATATGGGGTTGAA | |
| | ATTGGAGTTACATGACCTATGGGGT | |
| | CCGACTATTGTGGTCCAAATCACTG | |
| | GCAAGAGCAGCAAATATGCTCAAGG | |
| | ATTTTTTTCTTCCAATGGTACTTGT | |
| | TGCCTCCCAATCAGCAGATCTGCAC | |
| | CAGAGCTTGGGAAGCTTCTGTGGTC | |
| | CTGCTCAGCAACTATTGGTGACGCA | |
| | ACAGTTGTTATCCAATCAAGCGAGA | |
| | AGGGGGAACTCCTAAGGTCTGATGA | |
| | TCTCGAGATACGAGGTGCTGTGGCC | |
| | TCCAAGAAAGGTAGACTGAGCTCAT | |
| | TTCACCCCTTCAAAAAATGATGCAG | |
| | GACATAGTACAGAGAATGAAAGGGC | |
| | CATCAGACGTGCGAAAAAAACTAAA | |
| | TCTGAAAAAAACTGCCCAGACTCCA | |
| | CATTAATCTAGGTTGCAGGGAAATA | |
| | ATACCCGACATGCACAATACTATCA | |
| | CGGTCACCAGCAATCAGCAAAGTTG | |
| | ATCAATCACTATATAAGGAATCAAG | |
| | TGGGATAACAATTATTAATCCAATT | |
| | TCATAATTATAAAAAATTGCTTTAA | |
| | AGGTTACTGACGAGTCGGGGGCGAA | |
| | ACCTTGCCACTTAAGCTGCAGTCAA | |
| | TTTTAGAATCTACATATTGAATTAT | |
| | GGGTAAAATATCAATATATCTAATT | |
| | AATAGCGTGCTATTATTGCTGGTAT | |
| | ATCCTGTGAATTCGATTGACAATAC | |
| | ACTCGTTGCCCCAATCGGAGTCGCC | |
| | AGCGCAAATGAATGGCAGCTTGCTG | |
| | CATATACAACATCACTTTCAGGGAC | |
| | AATTGCCGTGCGATTCCTACCTGTG | |
| | CTCCCGGATAATATGACTACCTGTC | |
| | TTAGAGAAACAATAACTACATATAA | |
| | TAATACTGTCAACAACATCTTAGGC | |
| | CCACTCAAATCCAATCTGGATGCAC | |
| | TGCTCTCATCTGAGACTTATCCCCA | |
| | GACAAGATTAATTGGGGCAGTTATA | |
| | GGTTCAATTGCTCTTGGTGTTGCAA | |
| | CATCGGCTCAAATCACTGCTGCAGT | |
| | CGCTCTCAAGCAAGCACAAGATAAT | |
| | GCAAGAAACATACTGGCACTCAAAG | |
| | AGGCACTGTCCAAAACTAATGAGGC | |
| | GGTCAAGGAGCTTAGCAGTGGATTG | |
| | CAACAAACAGCTATTGCACTTGGTA | |
| | AGATACAGAGCTTTGTGAATGAGGA | |
| | AATTCTGCCATCTATCAACCAACTG | |
| | AGCTGCGAGGTGACAGCCAATAAAC | |
| | TTGGGGTGTATTTATCTCTGTATCT | |
| | CACAGAACTGACCACTATATTCGGT | |
| | GCACAGTTGACTAACCCTGCATTGA | |
| | CTTCATTATCATATCAAGCGCTGTA | |
| | CAACCTGTGTGGTGGCAACATGGCA | |
| | ATGCTTACTCAGAAGATTGGAATTA | |
| | AACAGCAAGACGTTAATTCGCTATA | |
| | TGAAGCCGGACTAATCACAGGACAA | |
| | GTCATTGGTTATGACTCTCAGTACC | |
| | AGCTGCTGGTCATCCAGGTCAATTA | |
| | TCCAAGCATTTCTGAGGTAACTGGT | |
| | GTGCGTGCGACAGAATTAGTCACTG | |
| | TTAGTGTAACAACAGACAAGGGTGA | |
| | AGGGAAAGCAATTGTACCCCAATTT | |
| | GTAGCTGAAAGTCGGGTGACTATTG | |
| | AGGAGCTTGATGTAGCATCTTGTAA | |
| | ATTCAGCAGCACAACCCTATACTGC | |
| | AGGCAGGTCAACACAAGGGCACTTC | |
| | CCCCGCTAGTGGCTAGCTGTCTCCG | |
| | AGGTAACTATGATGATTGTCAATAT | |
| | ACCACAGAGATTGGAGCATTATCAT | |
| | CCCGGTATATAACACTAGATGGAGG | |
| | GGTCTTAGTCAATTGTAAGTCAATT | |
| | GTTTGTAGGTGCCTTAATCCAAGTA | |
| | AGATCATCTCTCAAAATACAAATGC | |
| | TGCAGTAACATATGTTGATGCTACA | |
| | ATATGCAAAACAATTCAATTGGATG | |
| | ACATACAACTCCAGTTGGAAGGGTC | |
| | ACTATCATCAGTTTATGCAAGGAAC | |
| | ATCTCAATTGAGATCAGTCAGGTGA | |
| | CTACCTCCGGTTCTTTGGATATCAG | |
| | CAGTGAGATAGGGAACATCAATAAT | |
| | ACGGTGAATCGTGTGGAGGATTTAA | |
| | TCCACCAATCGGAGGAATGGCTGGC | |
| | AAAAGTTAACCCACACATTGTTAAT | |
| | AATACTACACTAATTGTACTCTGTG | |
| | TGTTAAGTGCGCTTGCTGTGATCTG | |
| | GCTGGCAGTATTAACGGCTATTATA | |
| | ATATACTTGAGAACAAAGTTGAAGA | |
| | CTATATCGGCATTGGCTGTAACCAA | |
| | TACAATACAGTCTAATCCCTATGTT | |
| | AACCAAACGAAACGTGAATCTAAGT | |
| | TTTGATCATTCAGGCCAAAACAGAG | |
| | GGTCTAGGCTCGGGTTAATAAAAGT | |
| | TCAATCAATGTTTGATTTATTAGGC | |
| | TTTCCCTACTAATTATTAATGTATT | |
| | TGTGATTATATGATAACGTTAAAAG | |
| | TCTTAAATATTTAATAAAAAATGTA | |
| | ACCTGGGGGCGACCTATTTACAGGC | |
| | TAGTATATATTAGGAAGTCCTCATA | |
| | TTGCACTATAATCTCAAACAATTAT | |
| | ATTACCTCGTATCCACCTTGTCTAA | |
| | AGACATCATGAGTAACATTGCATCC | |
| | AGTTTAGAAAATATTGTGGAGCAGG | |
| | ATAGTCGAAAAACAACTTGGAGGGC | |
| | CATCTTTAGATGGTCCGTTCTTCTT | |
| | ATTACAACAGGATGCTTAGCCTTAT | |
| | CCATTGTTAGCATAGTTCAAATTGG | |
| | GAATTTGAAAATTCCTTCTGTAGGG | |
| | GATCTGGCGGACGAGGTGGTAACAC | |
| | CTTTGAAAACCACTCTGTCTGATAC | |
| | ACTCAGGAATCCAATTAACCAGATA | |
| | AATGACATATTCAGGATTGTTGCCC | |
| | TTGATATTCCATTGCAAGTAACTAG | |
| | TATCCAAAAAGACCTCGCAAGTCAA | |
| | TTTAGCATGTTGATAGATAGTTTAA | |
| | ATGCTATCAAATTGGGCAACGGGAC | |
| | CAACCTTATCATACCTACATCAGAT | |
| | AAGGAGTATGCAGGAGGAATTGGAA | |
| | ACCCTGTCTTTACTGTCGATGCTGG | |
| | AGGTTCTATAGGATTCAAGCAATTT | |
| | AGCTTAATAGAACATCCGAGCTTTA | |
| | TTGCTGGACCTACAACGACCCGAGG | |
| | CTGTACAAGAATACCCACTTTTCAC | |
| | ATGTCAGAAAGTCATTGGTGCTACT | |
| | CACACAACATCATCGCTGCTGGCTG | |
| | TCAAGATGCCAGTGCATCTAGTATG | |
| | TATATCTCAATGGGGGTTCTCCATG | |
| | TGTCTTCATCTGGCACTCCTATCTT | |
| | TCTTACTACTGCAAGTGAACTGATA | |
| | GACGATGGAGTTAATCGTAAGTCAT | |
| | GCAGTATTGTAGCAACCCAATTCGG | |
| | CTGTGACATTTTGTGCAGTATTGTC | |
| | ATAGAGAAGGAGGGAGATGATTATT | |
| | GGTCTGATACTCCGACTCCAATGCG | |
| | CCACGGCCGTTTTTCATTCAATGGG | |
| | AGTTTTGTAGAAACCGAACTACCCG | |
| | TGTCCAGTATGTTCTCGTCATTCTC | |
| | TGCCAACTACCCTGCTGTGGGATCA | |
| | GGCGAAATTGTAAAAGATAGAATAT | |
| | TATTCCCAATTTACGGAGGTATAAA | |
| | GCAGACTTCACCAGAGTTTACCGAA | |
| | TTAGTGAAATATGGACTCTTTGTGT | |
| | CAACACCTACAACTGTATGTCAGAG | |
| | TAGCTGGACTTATGACCAGGTAAAA | |
| | GCAGCGTATAGGCCAGATTACATAT | |
| | CAGGCCGGTTCTGGGCACAAGTGAT | |
| | ACTCAGCTGCGCTCTTGATGCAGTC | |
| | GACTTATCAAGTTGTATTGTAAAGA | |
| | TTATGAATAGCAGCACAGTGATGAT | |
| | GGCAGCAGAAGGAAGGATAATAAAG | |
| | ATAGGGATTGATTACTTTTACTATC | |
| | AGCGGTCATCTTCTTGGTGGCCATT | |
| | GGCATTTGTTACAAAACTAGACCCG | |
| | CAAGAGTTAGCAGACACAAACTCGA | |
| | TATGGCTGACCAATTCCATACCAAT | |
| | CCCACAATCAAAGTTCCCTCGGCCT | |
| | TCATATTCAGAAAATTATTGCACAA | |
| | AGCCAGCAGTTTGCCCTGCTACTTG | |
| | TGTCACTGGTGTATACTCTGATATT | |
| | TGGCCCTTGACCTCATCTTCATCAC | |
| | TCCCGAGCATAATTTGGATCGGCCA | |
| | GTACCTTGATGCCCCTGTTGGAAGG | |
| | ACTTATCCCAGATTTGGAATTGCAA | |
| | ATCAATCACACTGGTACCTTCAAGA | |
| | AGATATTCTACCCACCTCCACTGCA | |
| | AGTGCGTATTCAACCACTACATGTT | |
| | TTAAGAATACTGCCAGGAATAGAGT | |
| | GTTCTGCGTCACCATTGCTGAATTT | |
| | GCAGATGGGTTGTTTGGAGAGTACA | |
| | GGATAACACCTCAGTTGTATGAATT | |
| | AGTGAGAAATAATTGAATCACGATA | |
| | ATTTTGGGACTCATTTAATTGCAGA | |
| | GTGAAATTGTCATCTTAGGAAATAA | |
| | TCAATTCCATGATTTTTATTGAACA | |
| | TGATCAAGCAATCATGTGGGAAATT | |
| | TATTATCACATAACTTCTAATAGTT | |
| | TTAAATGACGAATTAAGAAAAAATG | |
| | GAGGGCGACCTCTACACAAACATGG | |
| | ATGTAAAACAAGTTGACCTAATAAT | |
| | ACAACCCGAGGTTCATCTCGATTCA | |
| | CCCATCATATTGAATAAACTGGCAC | |
| | TATTATGGCGCTTGAGTGGTTTACC | |
| | CATGCCTGCAGACTTACGACAAAAA | |
| | TCCGTAGTGATGCACATCCCAGACC | |
| | ACATCTTAGAAAAATCAGAATATCG | |
| | GATCAAGCACCGTCTAGGGAAAATC | |
| | AAGAGTGACATAGCACATTACTGTC | |
| | AGTATTTTAATATTAATTTGGCAAA | |
| | TCTTGATCCGATAACCCACCCCAAA | |
| | AGTTTGTATTGGTTATCCAGACTAA | |
| | CAATAGCTAGTGCTGGAACCTTTAG | |
| | ACATATGAAAGATAGAATCTTATGT | |
| | ACAGTTGGCTCCGAATTCGGACACA | |
| | AAATTCAAGATTTATTTTCACTGCT | |
| | GAGCCATAAATTAGTAGGTAACGGT | |
| | GATTTATTTAATCAAAGTCTCTCAG | |
| | GTACACGTTTGACTGCGAGTCCGTT | |
| | ATCCCCTTTATGCAATCAATTTGTC | |
| | TCTGACATCAAGTCTGCAGTCACGA | |
| | CACCCTGGTCAGAAGCTCGTTGGTC | |
| | TTGGCTTCATATCAAACAAACAATG | |
| | AGATACCTGATAAAACAATCACGCA | |
| | CTACAAATTCAGCTCATTTAACAGA | |
| | AATTATAAAAGAGGAATGGGGTTTA | |
| | GTAGGTATTACTCCAGATCTTGTCA | |
| | TTCTTTTTGACAGAGTCAATAATAG | |
| | TCTAACTGCATTAACATTTGAGATG | |
| | GTTCTAATGTATTCAGATGTATTAG | |
| | AATCCCGTGACAATATTGTGCTAGT | |
| | GGGGCGATTATCTACTTTTCTGCAG | |
| | CCAGTAGTTAGTAGACTGGAGGTGT | |
| | TGTTTGATCTAGTAGATTCATTGGC | |
| | AAAAACCTTAGGTGACACAATATAC | |
| | GAAATTATTGCGGTGTTAGAGAGCT | |
| | TGTCTTATGGGTCCGTTCAACTACA | |
| | TGATGCAAGTCACTCTCATGCAGGG | |
| | TCTTTCTTTTCATTTAACATGAATG | |
| | AACTTGATAACACACTATCAAAGAG | |
| | GGTGGATCCGAAACACAAGAACACC | |
| | ATAATGAGCATTATAAGACAATGCT | |
| | TTTCTAATCTAGATGTTGATCAAGC | |
| | TGCAGAGATGCTATGCCTGATGAGA | |
| | TTATTTGGACACCCAATGTTAACTG | |
| | CACCGGATGCAGCAGCCAAAGTAAG | |
| | GAAAGCAATGTGTGCTCCAAAACTT | |
| | GTTGAACATGACACCATCTTGCAGA | |
| | CATTATCCTTCTTCAAGGGAATAAT | |
| | TATAAATGGGTACAGAAGATCACAC | |
| | TCTGGCCTGTGGCCCAATGTAGAGC | |
| | CGTCTTCAATCTATGATGATGATCT | |
| | CAGACAGCTGTACTTAGAGTCAGCA | |
| | GAGATTTCCCATCATTTCATGCTTA | |
| | AAAACTACAAGAGTTTGAGCATGAT | |
| | AGAATTCAAGAAGAGCATAGACTAC | |
| | GATCTTCACGACGACTTAAGTACTT | |
| | TCTTAAAGGATAGAGCAATTTGCCG | |
| | GCCAAAATCCCAGTGGGATGTTATA | |
| | TTCCGTAAGTCTTTACGCAGATCCC | |
| | ACACGCGGTCCCAGTATATGGACGA | |
| | AATTAAGAGCAACCGATTGCTAATT | |
| | GATTTTCTTGATTCTGCTGATTTTG | |
| | ACCCTGAAAAGGAATTTGCATATGT | |
| | AACCACAATGGATTATTTGCACGAT | |
| | AATGAATTTTGTGCTTCATATTCTC | |
| | TAAAGGAAAAGGAGATCAAAACTAC | |
| | CGGGAGGATATTTGCAAAAATGACA | |
| | CGCAATATGAGAAGTTGCCAAGTGA | |
| | TACTTGAATCTCTGTTATCAAAACA | |
| | TATATGCAAGTTCTTCAAAGAGAAC | |
| | GGCGTTTCGATGGAGCAATTGTCAT | |
| | TGACCAAGAGTCTACTTGCAATGTC | |
| | TCAACTCTCACCAAAAGTCTCGACT | |
| | CTGCAGGACACTGCATCACGTCATG | |
| | TAGGCAACTCAAAATCTCAGATCGC | |
| | AACCAGCAACCCATCTCGGCATCAC | |
| | TCAACAACCAATCAGATGTCACTCT | |
| | CAAATCGGAAAACGGTTGTAGCAAC | |
| | TTTCTTAACAACTGATTTGGAAAAA | |
| | TACTGCCTGCAGTGGCGATACTCGA | |
| | CTATTAAGTTGTTTGCACAAGCTCT | |
| | AAATCAACTCTTTGGGATTGATCAC | |
| | GGATTTGAATGGATACATTTAAGAC | |
| | TCATGAACAGCACCTTATTTGTCGG | |
| | TGATCCTTACTCGCCTCCTGAAGAT | |
| | CCAACACTAGAGGATATAGATAAAG | |
| | CACCAAATGACGATATCTTCATAGT | |
| | TTCTCCAAGGGGAGGCATAGAGGGT | |
| | TTATGTCAGAAGATGTGGACCATGA | |
| | TATCAATTAGTGCGATACACTGTGT | |
| | AGCAGAGAAAATTGGTGCACGAGTG | |
| | GCAGCAATGGTGCAGGGTGATAATC | |
| | AAGTAATAGCTATCACCAAAGAACT | |
| | ATTCAGAGGAGAGAAAGCCTGTGAT | |
| | GTCAGAGATGAGTTAGACGAGCTCG | |
| | GTCAGGTGTTTTTTGATGAGTTCAA | |
| | GAGGCACAATTATGCAATTGGACAC | |
| | AACCTTAAGCTAAATGAGACAATAC | |
| | AAAGCCAATCCTTTTTTGTATATTC | |
| | CAAACGAATATTCTTTGAAGGGCGA | |
| | TTGCTTAGTCAAGTCCTCAAAAATG | |
| | CTGCCAAGTTATGTATGGTTGCTGA | |
| | CCATCTAGGTGAAAACACAGTATCT | |
| | TCCTGTAGCAACCTGAGCTCTACAA | |
| | TTGCCCGGTTGGTGGAAAATGGGTT | |
| | TGAGAAGGACACTGCTTTTGTGTTG | |
| | AACCTAGTCTACATCATGACTCAAA | |
| | TTCTTTTTGATGAGCATTACTCGAT | |
| | TGTATGCGATCACAATAGTGTCAAA | |
| | AGCTTGATCGGATCAAAAAACTATC | |
| | GGAATCTATTGTACTCATCTCTAAT | |
| | ACCAGGTCAGCTCGGTGGTTTCAAC | |
| | TTCCTCAATATAAGTCGGTTGTTCA | |
| | CTAGGAATATAGGTGACCCAGTAAC | |
| | ATGTAGTCTGTCTGATCTCAAATGC | |
| | TTCATAGCCGCAGGTCTCCTTCCAC | |
| | CCTATGTACTTAAAAATGTGGTTCT | |
| | GCGTGAGCCTGGTCCTGGGACATGG | |
| | TTGACGTTGTGCTCTGATCCTTACA | |
| | CCCTTAACATACCATACACACAGCT | |
| | ACCAACCACATATCTCAAAAAGCAC | |
| | ACCCAGCGATCGTTGCTTTCACGTG | |
| | CAGTAAATCCTTTATTAGCAGGTGT | |
| | ACAAGTGCCAAATCAGCATGAGGAA | |
| | GAAGAGATGTTGGCTCGCTTTCTCC | |
| | TTGATCGTGAATATGTGATGCCCCG | |
| | CGTTGCTCATGTAACACTAGAAACA | |
| | TCGGTCCTTGGCAAACGGAAACAAA | |
| | TCCAAGGCTTAATTGATACAACTCC | |
| | AACTATCATTAGAACATCTCTAGTC | |
| | AATCTACCAGTGTCTAGGAAGAAAT | |
| | GCGAAAAAATAATCAATTATTCTCT | |
| | CAATTATATTGCTGAGTGTCATGAC | |
| | TCCTTACTTAGTCAGATCTGCTTCA | |
| | GTGATAATAAGGAATACTTGTGGTC | |
| | CACCTCCTTAATATCAGTTGAGACC | |
| | TGTAGTGTGACAATTGCGGACTATT | |
| | TGAGAGCTGTCAGCTGGTCTAATAT | |
| | ATTAGGGGGAAGAAGCATATCCGGG | |
| | GTGACTACACCTGATACTATTGAAT | |
| | TAATTCAAGGTTGTTTAATAGGTGA | |
| | AAATTCCAGTTGTACTCTTTGTGAA | |
| | TCGCATGACGACGCATTCACATGGA | |
| | TGCACTTGCCTGGCCCACTTTACAT | |
| | CCCTGAACCATCAGTTACTAACTCT | |
| | AAAATGCGTGTGCCATATCTGGGTT | |
| | CAAAAACAGAGGAGCGTAAAACAGC | |
| | TTCAATGGCAGCAATAAAAGGAATG | |
| | TCACATCACCTGCGTGCAGTCTTAA | |
| | GAGGTACATCCGTATTTATTTGGGC | |
| | ATCTGGGGACACAGATATTAATTGG | |
| | GATAATGCATTGCAGATTGCCCAAT | |
| | CACGGTGTAACATCACATTGGATCA | |
| | AATGAGATTACTTACACCAATTCCT | |
| | AGCAGTTCAAATATCCAACGTAGAC | |
| | TCGATGACGGAATCAGCACGCAGAA | |
| | ATTTACTCCTGCAAGCCTTGCTCGA | |
| | ATCACATCCTCTGTTCACATCTGTA | |
| | ATGACAGCCAAAGGTTAGAGAAGGA | |
| | TGGCTCCTCTGTCGACTCAAACTTG | |
| | ATTTACCAGCAAATTATGTTACTTG | |
| | GACTCAGCATCTTTGAAACAATGTA | |
| | CTCAATGGACCAAAAGTGGGTATTC | |
| | AATAACCATACCTTACATTTGCACA | |
| | CTGGACACTCCTGTTGTCCAAGGGA | |
| | ACTAGACATAAGTTTAGTGAACCCG | |
| | CCAAGACATCAGACCCCGGAGCTGA | |
| | CTAGCACAACAACCAACCCGTTCCT | |
| | ATATGATCAGCTCCCACTAAATCAG | |
| | GATAATCTGACAACACTTGAGATTA | |
| | AGACATTCAAATTTAATGAGCTCAA | |
| | CATTGATGGTTTAGATTTTGGTGAA | |
| | GGAATACAATTATTGAGTCGTTGTA | |
| | CTGCAAGATTAATGGCAGAATGTAT | |
| | TCTAGAGGAGGGAATAGGCTCGTCA | |
| | GTTAAAAATGAAGCAATTGTCAATT | |
| | TTGATAATTCAGTCAATTGGATTTC | |
| | AGAGTGCCTAATGTGTGATATTCGC | |
| | TCACTTTGTGTTAATTTAGGTCAAG | |
| | AGATACTATGTAGCCTGGCATACCA | |
| | AATGTATTACTTGCGAATCAGGGGT | |
| | AGAAGGGCCATTCTTAATTACTTGG | |
| | ACACAACTTTGCAAAGGATCCCTGT | |
| | GATACAGTTAGCCAACATTGCACTC | |
| | ACCATTTCACACCCTGAGATATTTC | |
| | GCAGAATTGTCAACACCGGGATCCA | |
| | TAACCAGATTAAGGGCCCATATGTG | |
| | GCAACAACAGATTTCATAGCTGCAA | |
| | GTAGAGATATCATATTATCAGGTGC | |
| | AAGGGAGTATCTATCTTATCTAAGC | |
| | AGTGGACAGGAAGACTGTTACACAT | |
| | TCTTCAACTGTCAAGATGGGGATCT | |
| | TACTCCAAAAATGGAACAGTATCTT | |
| | GCAAGGAGGGCATGCCTTTTAACAT | |
| | TACTGTATAATACTGGGCACCAGAT | |
| | CCCCATTATCCGATCACTGACACCA | |
| | ATAGAGAAGTGCAAGGTGCTCACAG | |
| | AATACAATCAACAAATTGAGTATGC | |
| | AGATCAAGAGTTTAGCTCTGTATTG | |
| | AAAGTGGTCAATGCACTACTACAAA | |
| | ATCCTAATATAGATGCATTGGTTTC | |
| | AAATCTCTACTTCACCACCAGACGT | |
| | GTTTTATCAAACCTCAGATCATGTG | |
| | ATAAGGCTATATCATATATTGAATA | |
| | TTTGTACACTGAGGACTTCGGAGAA | |
| | AAAGAAGATACAGTACAATATGACA | |
| | TCATGACAACAAACGATATCATACT | |
| | TACTCATGGTCTATTCACACAGATC | |
| | GAAATATCTTACCAAGGGAGTAGTC | |
| | TCCATAAATTCCTAACTCCGGATAA | |
| | CGCGCCTGGATCATTGATCCCATTC | |
| | TCTATTTCACCAAATTCGCTTGCAT | |
| | GTGATCCTCTTCACCACTTACTCAA | |
| | GTCGGTCGGTACATCAAGCACAAGC | |
| | TGGTACAAGTATGCAATCGCCTATG | |
| | CAGTGTCTGAAAAGAGGTCGGCTCG | |
| | ATTAGGAGGGAGCTTGTACATTGGT | |
| | GAAGGGAGCGGAAGTGTGATGACTT | |
| | TGCTAGAGTATCTTGAGCCATCTGT | |
| | TGACATATTTTACAATTCACTCTTC | |
| | TCAAATGGTATGAACCCACCACAAC | |
| | GAAATTATGGGCTTATGCCACTACA | |
| | ATTTGTGAATTCGGTGGTTTATAAG | |
| | AACTTAACGGCTAAATCAGAATGTA | |
| | AGCTAGGATTTGTCCAGCAATTTAA | |
| | ACCGTTGTGGAGAGACATAGACATT | |
| | GAGACTAATGTTACAGATCCATCAT | |
| | TTGTCAATTTTGCATTGAATGAAAT | |
| | CCCAATGCAATCATTAAAACGAGTA | |
| | AATTGTGATGTGGAATTTGACCGTG | |
| | GTATGCCGATTGAACGGGTTATTCA | |
| | GGGTTACACTCATATCTTACTTGTT | |
| | GCTACTTACGGATTGCAGCAAGATT | |
| | CAATACTGTGGGTGAAAGTATATAG | |
| | GACATCTGAAAAAGTATTTCAGTTC | |
| | TTACTGAGTGCCATGATCATGATCT | |
| | TTGGTTATGTCAAAATCCACAGGAA | |
| | TGGTTATATGTCGGCAAAGGATGAG | |
| | GAGTACATATTGATGTCTGACTGCA | |
| | AGGAACCTGTAAACTATACAGCTGT | |
| | CCCTAACATTCTTACACGTGTAAGT | |
| | GATTTAGTGTCGAAGAATCTGAGTC | |
| | TTATCCATCCAGAAGACCTCAGAAA | |
| | GGTAAGGTGTGAAACAGATTCCCTG | |
| | AATTTGAAGTGCAATCATATTTATG | |
| | AGAAAATAATTGCTAGAAAAATTCC | |
| | ATTACAGGTGTCATCAACTGATTCT | |
| | TTGCTCCTCCAGTTAGGCGGTGTCA | |
| | TCAACTCGGTGGGCTCAACTGATCC | |
| | TAGAGAGGTTGCAACGTTATCTTCC | |
| | ATTGAGTGTATGGACTATGTTGTCT | |
| | CATCAATTGATTTGGCTATATTAGA | |
| | GGCAAATATTGTGATCTCAGAGAGT | |
| | GCTGATCTTGACCTCGCTTTAATGT | |
| | TAGGCCCATTCAACTTGAATAAGCT | |
| | TAAGAAAATTGACACAATCCTTAAG | |
| | TCAAGCACCTATCAGCTAATCCCGT | |
| | ATTGGTTGCGCTATGAGTACTCTAT | |
| | TAATCCGAGATCTTTGTCATTTCTA | |
| | ATCACTAAATTACAACAATGCCGAA | |
| | TTTCATGGTCAGATATGATAACAAT | |
| | CTCTGAATTTTGCAAGAAATCCAAG | |
| | CGGCCTATATTTATTAAACGAGTAA | |
| | TAGGGAATCAACGGCTGAAATCATT | |
| | CTTTAATGAAAGCTCAAGTATTGTT | |
| | TTGACCCGGGCTGAAGTCAAAGTCT | |
| | GTATAAAGTTCCTCGGTGCGATCAT | |
| | CAAGTTGAAATAATTTCTGTGTTTT | |
| | TTAAGGGGTATAGTATTCTAAGTTG | |
| | CACTTGAAGTAATATAGCTTGTAAT | |
| | CATTCGCTAGGGGATAGAATAATTC | |
| | CTATAATCTCTGAATATATATCTCT | |
| | AGGTTATAACAAATATATACATAAT | |
| | AAAATTGATTTTAAGAAAAAATCCG | |
| | ACTTTCAAAGAAGATTGGTGCCTGT | |
| | AATATTCTTCTTGCCAGATGATTAT | |
| | GGAGGGTCTAGCCTAACTTAAAACA | |
| | ATCGTATTCGATAGGGAAGAATGAC | |
| | ATATAAAGTAACTAATAAAAAATTG | |
| | TATTAGTGAAAATTACCGTATTTCC | |
| | TGTATTCCATTTCTGGT | |
| Avian | ACCAAACAAAGAAATTGTAAGATAC | SEQ ID |
| paramyxovir | GTTAAAGACCGAAGTAGCAACTGAC | NO: 12 |
| us 9 strain | TTCGTACGGGTAGAAGGATTGAATC | |
| duck/New | TCGAGTGCGAACACGACGCTGTGAT | |
| York/22/ | TCGAAGGTCCGTACTACCATCATGT | |
| 1978, | CCTCTATATTCAATGAGTATGAGAG | |
| complete, | TCTGCTTGAAAGTCAACTCAAACCG | |
| genome | ACGGGCTCGAACGTCTTAGGAGAGA | |
| Genbank: | AAGGTGACACTCCAAAAGTCGAGAT | |
| NC_025390.1 | CCCTGTATTTGTGCTCAACAGTGAC | |
| | AACCCTGAAGATCGCTGGAACTTTA | |
| | CTACCTTCTGTCTCAGAGTCGCTGT | |
| | GAGCGAGGATGCTAATAGGCCTTTG | |
| | CGTCAGGGGGCACTCATCTCTCTAC | |
| | TTTGCGCTCATTCTCAGGTGATGAA | |
| | GAATCATGTGGCCATAGCAGGAAAG | |
| | CAGGATGAGGCTCTGATTGTAGTTC | |
| | TAGAGATTGATACTATTAATGATGG | |
| | TGTTCCAGCCTTCAACAATAGGAGC | |
| | GGTGTCACAGAGGAACGAGCTCAGC | |
| | GTTTCGCTATGATAGCTCAAGCATT | |
| | ACCCCGTGCTTGTGCAAATGGGACA | |
| | CCGTTCACCGTCCAAGATGCAGAAG | |
| | ATGATCCAGTCGAAGACATAACAGA | |
| | CGCCCTTGATCGCATATTGTCAATC | |
| | CAGGCGCAAGTATGGGTGACCGTCG | |
| | CAAAATCCATGACAGCGTACGAGAC | |
| | TGCAGATGAATCAGAACAGAAGCGA | |
| | TTGACCAAGTATGTTCAGCAAGGTC | |
| | GAGTGCAGAAGAAATGCATGATCTA | |
| | CCCTGTATGTCGGAGCATGCTGCAG | |
| | CAGATCATAAGGCAATCTTTAGCAG | |
| | TCCGACGGTTCATTGTCAGTGAGCT | |
| | GAAACGAGCTCGGAATACAGCAGGA | |
| | GGAACATCCACGTATTATAACTTCG | |
| | TTGCTGATGTAGATTCCTACATTAG | |
| | GAATGCTGGGTTAACTGCATTCTTC | |
| | TTGACCCTTAAGTATGGTGTGAATA | |
| | CAAAGACTTCTGTCCTTGCCCTTAG | |
| | CAGCTTGGCAGGCGATCTTCAAACT | |
| | GTCAAACAGTTGATGCGGCTGTATA | |
| | AAGCCAAAGGAGATGATGCACCATA | |
| | CATGACTATACTGGGAGACGGAGAC | |
| | CAGATGAGATTTGCACCTGCTGAAT | |
| | ACGCACAGCTATACTCATACGCTAT | |
| | GGGAATGGCATCAGTCATAGACAAA | |
| | GGGACCTCAAGGTATCAGTACGCTC | |
| | GTGACTTCCTAAACCCCAGCTTCTG | |
| | GAGGCTGGGAGTGGAGTATGCCCAG | |
| | ACTCAAGGAAGCAACATCAACGAAG | |
| | AGATGGCATCAGAACTGAAACTCAG | |
| | CCCAATAGCTAGAAGGATGCTGACC | |
| | ACTGCCGTCACAAAAGTAGCAACCG | |
| | GAGCGTCTGATTATTCGGTACCTCA | |
| | GCATACAGCAGGAGTCCTAACTGGC | |
| | TTGAATTCAACAGACGGCAACCTTG | |
| | GGTCTCAGAAGCTGCCCACCTCAAT | |
| | TCAGCAGGATCAGAATGATGATACT | |
| | GCCATGTTGAACTTCATGAGGGCCG | |
| | TAGCACAAGGAATGAAGGAGACACC | |
| | AATTCAGGCTCCTCCCACCCCTGGA | |
| | TTCGGATCTCAACAGGCCGCAGACG | |
| | ACGATGACTCGCGGGATCAAGCAGA | |
| | CTCCTGGGGGCTCTAATGAAATACG | |
| | GAGGTTGACTCCAGCCCAAACGAAC | |
| | CTCTAGCAACTCCTAATCCCTCATC | |
| | CACCTACAAACTCCACATCTACATG | |
| | ACCAATCCGCTCACACAACACGGCG | |
| | GAAGACACCATCCATCCCCAACTGT | |
| | CCCAACCCGAAGAACATCCTCAACT | |
| | TAGCCCGCTAATTTCACGAACCATT | |
| | ACAAAAAACTTATCAACAGAAAAAA | |
| | CTACGGGTAGAACTGTCTGCCACTG | |
| | CGAGAAAGCAAACGCATCAACGCAG | |
| | TCAGCACTCATCGCAGCTCTCCATC | |
| | ACACCAATTCTAGCTCAGGCACACG | |
| | CCTCCAGAGAGAACCATGGCATCCT | |
| | TCACAGACGACGAGATATCAGATCT | |
| | GATGGAACAAAGTGGTCTTGTAATA | |
| | GATGAGATCATGACATCCCAAGGGA | |
| | TGCCTAAAGAGACCCTAGGGCGAAG | |
| | TGCAATCCCACCAGGGAAAACTCAG | |
| | GCCCTAACTGATGCCTGGGAGAAAC | |
| | ACAACAAGTCACAGAGATCCAATGC | |
| | GGATCACAGCACCGGATCAAATAAC | |
| | AAAACTGATGTCAACACACCCCACA | |
| | ATGCTGAGCCGCCACAATCCACCGG | |
| | CGATCCCTCCGCATCTCCAGAAATG | |
| | GACGGCGACACAACCCCACTCCCAA | |
| | AGCAGGAAACCGCCGAAAAGCACCC | |
| | CTGCAAAGAAGGGGCCACTGGAGGG | |
| | CTGCTGGATATGCTTGACCGGATTG | |
| | CTGCCAAGCAGGATAGAGCTAAAAA | |
| | AGGGCTCAATCCGAGATCACAAGAC | |
| | ACGGGCACCCTGCACTCAGGCCAAT | |
| | TCCCTACGCAGACGCAAGACCCGAC | |
| | ATCCCGCCGATCAACCAACTCATCG | |
| | GGACACAGCATGGAGTCCAGAACGC | |
| | CCGCCCAGCTGCCAATCCCGAGGAG | |
| | AGACGACAGCCCGCATCAGGTAAGA | |
| | AGAGAGGAGGAGGGCATCGCAGAGA | |
| | ACACAGCATGGTCTGGAATGCAAAC | |
| | GGGATTGTCACCATCAGCTGGTGCA | |
| | ACCCAGTTTGCTCTCCAGTCACCTA | |
| | CGAACCAAGAGAATTCACATGTTCA | |
| | TGCGGGAGCTGCCCTACAGAATGCC | |
| | GACTTTGTGCAGGCTCTCATAGGGA | |
| | TATTAGAAAGCATTCAGCAGAGAGT | |
| | GAGTAAAATGGAATATCAGATGGAT | |
| | TTAGTCCTGCGTCACCTGTCTAGTA | |
| | TGCCAGCCATTCGAAATGACATTCA | |
| | ACAAGTTAAGACCGCTATGGCAGTG | |
| | CTTGAGGCCAACATTGGGATGATGA | |
| | AAATCCTTGACCCTGGATCAGCACA | |
| | TATTTCTTCGCTCAATGATCTTCGA | |
| | GCAGTTGCAAGGTATCATCCAGTCC | |
| | TTGTAGCAGGCCCCGGTGACCCCAA | |
| | TAAAACAATTGCTGATGATAAAACC | |
| | ATCACTGTCAATCGGCTCTCCCAGC | |
| | CGGTAACTGATCAGCGCAGCTTGGT | |
| | AAGAGAACTCACACCCCCTTCCGGT | |
| | GATTTCGAGGCAGAAAAATGCGCAA | |
| | TCAAGGCGTTATTAGCTGCGAGACC | |
| | ACTACATCCATCGGCTGCAAAACGA | |
| | ATGTCTGATAGGTTAGATGCAGCCA | |
| | AGACATGTGAAGAATTGAGGAAGGT | |
| | GAAGAGACAGATTCTGAATAACTGA | |
| | CCCAAATAGTGTGGTTTCCGCCAAT | |
| | GATCAAGCGTGATCCGCCTTGGACA | |
| | ACTTTTTTGCCGATCTTAAGGAGAG | |
| | ACAAATCAATTTACACCGATCTAAA | |
| | ATATCATCAGACACCCTCAAATCAA | |
| | GAAAACATAGATGACAGTCTGCTTG | |
| | ACTCATCTCTTGCATCTGATGCTAT | |
| | CAATTGCCCTAAAATACCACCTGAC | |
| | ATAAATACCAGATTATCTCTAGACC | |
| | TCCTTGGTTGTTAAGAAAAAAAAGT | |
| | AAGTACGGGTAGAAACAGGACTCAA | |
| | CCGACCTACCACCATGGATGCTTCT | |
| | AGGATGATCAGTCTATATGTAGACC | |
| | CCACTAGCAGTTCTAGTTCAATACT | |
| | CGCATTCCCAATAGTCATGGAAGCC | |
| | ACAGGAGACGGACGAAAGCAAATTT | |
| | CACCCCAATATCGCATTCAGAGATT | |
| | AGATCACTGGTCAGACAGCAGTCGA | |
| | GATGCAGTATTCATCACCACATATG | |
| | GGTTTATATTTGGATACCCTAAATC | |
| | ACGTGCTGATCGAGGCCAGCTTAAT | |
| | GAAGAAATTAGGCCTGTGCTGCTCT | |
| | CTGCTGCAACGCTATGTCTGGGCAG | |
| | TGTGGCGAATACTGGAGATCAGGTT | |
| | GCAATTGCTCGGGCATGCTTGTCAC | |
| | TACAAATATCTTGCAAAAAGAGTGC | |
| | TACTAGTGAGGAGAAAATGATATTT | |
| | GCAATCACCCAAGCTCCGCAGATTT | |
| | TACAATCATGTCGTGCTGTTTCGCA | |
| | AAAATTCGTCTCCGTTGGATCAAAT | |
| | AAATGTGTGAAAGCACCTGAAAGAA | |
| | TCGAGGGAGGCCAGCAGTATGACTA | |
| | TAAGGTCAACTTCGTGTCTCTCACT | |
| | ATAGTACCAAAAGATGACGTATATA | |
| | GGGTCCCAAAACCTGTCCTATCAGT | |
| | CAGCAGTCCCACTCTATTCCGCCTT | |
| | GCCCTGAGTGTTAACATCGCAATCG | |
| | ACATCAATGCCGACAATCCTTTGTC | |
| | TAAGACGCTTATTAAGACCGAAAGC | |
| | GGCTTTGAAGCAAATTTGTTCCTGC | |
| | ATGTGGGTATTCTCTCAAACATTGA | |
| | CAAGCGGGGAAAGAAGGTGACGTTC | |
| | GAGAAGTTAGAGAAGAAAATCCGGC | |
| | GGATGGAACTGACTGCAGGATTAAG | |
| | TGATATGTTTGGTCCGTCCATCATC | |
| | CTGAAGGCCAAAGGGCCGAGGACAA | |
| | AGTTGATGTCAGCATTCTTTTCTAA | |
| | TACGGGAACAGCGTGTTATCCGATC | |
| | GCACAAGCATCTCCTCCAGTATCGA | |
| | AGATCTTGTGGAGCCAAAGCGGACA | |
| | CCTCCAGGAGGTTAAGATACTTGTA | |
| | CAATCGGGAACCTCGAAAATGATTG | |
| | CATTAACAGCCGATCAAGAAATCAC | |
| | AACAACAAAGCTCGATCAGCACGCC | |
| | AAGATTCAATCATTTAACCCATTCA | |
| | AAAAGTAAGTTGCATGGCTCACGAA | |
| | TAGCTCAGGTCTTCTTGCCTTAAAA | |
| | TCAGCCAATGAATATGTGATAGGAT | |
| | ATTCAGTGTCTCGAATCATTACCGA | |
| | TCAAAAAACCCCATTAAATCATACA | |
| | CCTGATCATTAGACAAGAGGTAATC | |
| | CAAATAGCATTAAAAAAAATCCCCA | |
| | AAAGAATTAAAACTAAAACACAGCA | |
| | CGGGTAGAAAGTGAGCTGTATATCA | |
| | CTCAATCCACAATCTACCATAGTGA | |
| | CACAATGGGGTACTTCCACCTATTA | |
| | CTTATACTAACAGCGATTGCCATAT | |
| | CTGCGCACCTCTGCTATACCACGAC | |
| | ATTGGATGGTAGAAAACTGCTTGGT | |
| | GCAGGCATAGTGATAACAGAAGAGA | |
| | AGCAAGTTAGGGTGTACACAGCTGC | |
| | GCAATCAGGAACAATTGTCTTAAGG | |
| | TCTTTCCGTGTGGTCTCCTTAGACA | |
| | GATACTCGTGCATGGAATCCACTAT | |
| | TGAGTCATATAACAAGACTGTATAT | |
| | AACATACTTGCACCTCTGGGCGATG | |
| | CAATCCGCCGAATACAGGCAAGTGG | |
| | TGTATCGGTTGAGCGTATCCGAGAG | |
| | GGCCGCATATTTGGTGCCATCCTTG | |
| | GGGGAGTTGCCTTAGGTGTAGCCAC | |
| | CGCAGCACAGATAACAGCTGCAATT | |
| | GCTTTGATTCAGGCTAACGAGAACG | |
| | CAAAAAACATCCTGCGTATTAAAGA | |
| | CAGTATAACTAAGACCAACGAGGCA | |
| | GTGAGAGATGTAACTAATGGCGTGT | |
| | CGCAGTTAACTATCGCTGTAGGTAA | |
| | ATTACAGGACTTCGTCAATAAGGAA | |
| | TTCAATAAGACAACTGAGGCCATTA | |
| | ATTGTGTACAGGCAGCTCAACAATT | |
| | AGGTGTGGAGCTAAGCCTCTATCTG | |
| | ACCGAGATCACTACAGTCTTCGGAC | |
| | CTCAGATAACCTCTCCTGCTTTAAG | |
| | CAAATTGACTATCCAAGCGCTGTAT | |
| | AATTTGGCGGGCGTAAGCTTGGATG | |
| | TACTACTGGGAAGGCTCGGAGCAGA | |
| | CAATTCACAGTTATCATCTTTGGTT | |
| | AGTAGTGGTCTTATTACCGGACAGC | |
| | CCATTCTCTACGACTCGGAATCTCA | |
| | AATATTGGCACTGCAAGTGTCACTA | |
| | CCCTCCATTAGTGACTTAAGGGGAG | |
| | TGAGAGCGACATACTTAGACACGTT | |
| | GGCTGTCAACACTGCAGCAGGACTT | |
| | GCATCTGCTATGATTCCAAAGGTAG | |
| | TAATCCAATCTAATAATATAGTTGA | |
| | AGAATTAGATACTACAGCATGTATA | |
| | GCAGCAGAAGCTGACTTATACTGTA | |
| | CGAGGATTACTACATTCCCCATTGC | |
| | GTCGGCTGTATCAGCCTGCATTCTT | |
| | GGGGATGTATCGCAATGCCTTTATT | |
| | CAAAGACTAATGGCGTCTTAACCAC | |
| | TCCATATGTAGCAGTAAAGGGGAAA | |
| | ATTGTAGCCAATTGTAAGCATGTCA | |
| | CATGTAGGTGTGTAGATCCTACATC | |
| | CATCATATCTCAAAATTACGGTGAA | |
| | GCAGCGACTCTTATCGATGATCAGC | |
| | TATGCAAGGTAATCAACTTAGATGG | |
| | TGTGTCCATACAGCTGAGCGGCACA | |
| | TTTGAATCGACTTATGTGCGCAACG | |
| | TCTCGATAAGTGCAAACAAGGTCAT | |
| | TGTCTCAAGCAGTATAGATATATCT | |
| | AATGAGCTGGAGAATGTTAACAGCT | |
| | CTTTAAGTTCGGCTCTGGAAAAACT | |
| | GGATGAAAGTGACGCTGCGCTAAGC | |
| | AAAGTAAATGTTCACTTAACTAGCA | |
| | CCTCAGCTATGGCCACATACATTGT | |
| | TCTAACTGTAATTGCTCTTATCTTG | |
| | GGGTTTGTCGGCCTAGGATTGGGTT | |
| | GCTTTGCTATGATAAAAGTAAAGTC | |
| | TCAAGCAAAGACACTACTATGGCTT | |
| | GGTGCACATGCTGACCGATCATATA | |
| | TACTCCAGAGTAAGCCGGCTCAATC | |
| | GTCCACATAATACAACAACAATCAA | |
| | TCCTGACTATCATATAATACATGAA | |
| | TCATTTCTTCTTCCGATTATAAAAA | |
| | AATAAGAAACCTAATTAGGCCAATA | |
| | CGGGTAGAACAGGCTTCCACCCCGT | |
| | ATTTCTTCGGCTGTGATCCTGTACC | |
| | TGAGTTCTTCCCACCAACACCAGGA | |
| | CCTCTCCTAAATTGCATCACCATGG | |
| | AATCAGGAATCAGCCAGGCATCTCT | |
| | TGTCAATGACAACATAGAATTAAGG | |
| | AATACGTGGCGCACGGCCTTCCGTG | |
| | TGGTCTCCTTATTACTCGGCTTCAC | |
| | CAGCTTGGTGCTCACTGCTTGCGCT | |
| | TTACACTTCGCTTTGAATGCCGCTA | |
| | CCCCTGCGGATCTCTCTAGTATCCC | |
| | AGTCGCTGTTGACCAAAGTCATCAT | |
| | GAAATTCTACAAACCTTGAGTCTGA | |
| | TGAGCGACATTGGCAATAAGATTTA | |
| | CAAGCAGGTAGCACTAGATAGTCCA | |
| | GTGGCGCTGCTCAACACTGAATCAA | |
| | CCTTAATGAGCGCAATTACATCACT | |
| | ATCTTATCAGATTAACAATGCAGCG | |
| | AATAACTCAGGTTGTGGCGCCCCTG | |
| | TGCATGATAAGGATTTTATCAATGG | |
| | AGTGGCAAAGGAATTATTTGTAGGG | |
| | TCTCAATACAATGCCTCGAACTATC | |
| | GACCCTCCAGGTTCCTTGAGCATCT | |
| | AAATTTCATCCCCGCCCCTACTACG | |
| | GGAAAAGGTTGCACCAGAATTCCGT | |
| | CCTTTGATCTAGCTGCAACACATTG | |
| | GTGTTATACTCACAATGTGATTCTT | |
| | AATGGTTGTAATGATCATGCTCAAT | |
| | CTTATCAATACATATCCCTCGGGAT | |
| | ACTCAAGGTGTCAGCCACGGGAAAC | |
| | GTGTTCTTATCTACTCTCAGATCTA | |
| | TCAACCTGGATGATGATGAAAACCG | |
| | GAAATCATGTAGCATATCAGCAACG | |
| | CCACTAGGGTGTGACTTACTTTGTG | |
| | CTAAAGTCACTGAGAGAGAAGAGGC | |
| | AGATTACAATTCAGATGCAGCGACG | |
| | AGATTAGTTCATGGCAGGTTAGGTT | |
| | TTGATGGGGTATACCATGAGCAGGC | |
| | CCTGCCTGTAGAATCATTGTTCAGT | |
| | GACTGGGTTGCAAACTATCCGTCAG | |
| | TCGGCGGAGGCAGTTACTTTGATAA | |
| | TAGGGTATGGTTTGGCGTGTATGGG | |
| | GGGATCAGACCTGGCTCTCAGACTG | |
| | ATCTGCTCCAGTCTGAGAAGTACGC | |
| | GATATATCGTAGGTACAATAATACC | |
| | TGCCCTGATAATAATCCCACCCAGA | |
| | TTGAGCGGGCCAAATCATCTTATCG | |
| | TCCGCAGCGGTTTGGCCAGCGGCTT | |
| | GTACAACAAGCAATTCTATCAATTA | |
| | GAGTGGAGCCATCTTTGGGTAATGA | |
| | TCCTAAACTATCTGTGTTAGATAAT | |
| | ACAGTCGTGTTGATGGGGGCGGAAG | |
| | CAAGGATAATGACATTTGGCCACGT | |
| | GGCATTAATGTATCAAAGAGGGTCA | |
| | TCATATTTTCCTTCTGCACTATTAT | |
| | ACCCTCTCAGTTTAACAAATGGTAG | |
| | TGCAGCAGCATCCAAGCCTTTCATA | |
| | TTCGAGCAATATACAAGGCCAGGTA | |
| | GCCCACCTTGTCAGGCCACTGCAAG | |
| | ATGTCCAAATTCATGTGTTACTGGT | |
| | GTCTACACAGACGCATACCCGTTAT | |
| | TTTGGTCTGAAGATCATAAAGTGAA | |
| | TGGTGTATATGGTATGATGTTAGAT | |
| | GACATCACATCACGGTTAAACCCGG | |
| | TAGCAGCTATATTTGATAGGTATGG | |
| | TAGGAGTAGAGTGACTAGGGTTAGC | |
| | AGTAGCAGCACGAAGGCAGCTTACA | |
| | CTACAAATACATGCTTTAAGGTTGT | |
| | CAAAACAAAGAGAGTATACTGCTTG | |
| | AGCATTGCCGAGATAGAGAATACAC | |
| | TGTTTGGAGAATTCAGAATAACCCC | |
| | TTTACTCTCCGAGATAATATTTGAC | |
| | CCAAACCTTGAACCCTCAGACACGA | |
| | GCCGTAACTGAGGAAAATCCGTTCT | |
| | GGCAGACAGTGGTTGGATAGACCTT | |
| | GCGTCGATAGCCCTCACTGTTGGCA | |
| | CTGCGTCGTCCCTATATTCAAACAC | |
| | CACATTAGCGGAGTATACAGATAGT | |
| | CGGCCATGATGAATCAAATGTCATG | |
| | CGATTTGAGCATAACCGAAGCAGAA | |
| | TCAGGATATACCCGGCTCTACCATA | |
| | TCAGGGAGAACAGCTGGTAAGCTGT | |
| | AATCCTCAATAATCCTAAAAACTGC | |
| | AGGTAATACAAAAGGATCAGCCTAT | |
| | AGGGAGCTTCAACAATCGTTAGAAA | |
| | AAAACGGGTAGAACATGGATAATCC | |
| | AGGACAATCTCGCCCTGATCATCAA | |
| | GTGATTCTACCCGAAGCGCATCTTT | |
| | CCTCACCGATCGTAAGGCATAAGTT | |
| | ATATTATTTCTGGAGACTAACAGGA | |
| | GTACCACTACCCCACTCAGCAGAAT | |
| | TTGATACGCTAGTCCTATCCAGACC | |
| | ATGGAACAAAATATTGCAGAGCAAC | |
| | TCGCCAGAAGTACTGAGGATGAAGC | |
| | GGCTAGGTGCGAACGTCCACGCGAC | |
| | TCTAGATCACTCTCGACCAATAAAG | |
| | GCTTTGATCCACCCGGAGACTTTAG | |
| | CATGGCTAACTGATCTGTCTATAGG | |
| | GGTATCTATCTCTAGATTTAGAGGA | |
| | ATAGAAAAGAAAGTATCTCGCCTGC | |
| | TCCATGACAATAGAGAGAAATTTTG | |
| | TACACTTGTTTCTCAGATTCATGAA | |
| | GGATTGTTCGGTGGTGTAGGAGGGG | |
| | TTCGGAATAATCTGTCACCAGAGTT | |
| | TGAAAGTTTGCTCAATGGAACTAAC | |
| | TTCTGGTTTGGCGGGAAATATTCAA | |
| | ACACAAAATTCACTTGGCTTCACAT | |
| | TAAACAATTGCAGAGACATCTTATA | |
| | CTCACAGCGCGTATGAGATCTGGGC | |
| | AGCAACTTTACATCCAATTAAAGCA | |
| | TACAAGGGGTTATGTCCATATAACT | |
| | CCAGAGTTAACTATGATTACATGCA | |
| | ACGGAAAAAACCTTGTTACAGCACT | |
| | TACACCTGAGATGGTCTTAATGTAT | |
| | AGTGACATGCTAGAAGGAAGAGATA | |
| | TGGTCATAAGTGTTGCACAGCTTGT | |
| | GAATGGCCTGAATGTCCTAGCAGAT | |
| | AGGATTGAGTGTCTTCTTGACTTGA | |
| | TTGACCAATTGGCGTGCTTGATAAA | |
| | GGATGCTATATATGAAATAATTGGG | |
| | ATTTTGGAGGGTTTAGCTTATGCAG | |
| | CAGTCCAGCTGCTGGAGCCGTCCGG | |
| | AAAATTCGCAGGGGATTTCTTTGAA | |
| | TTCAATCTCAGAGAGATAGCTGCCA | |
| | TATTGCGAGAACACATAGACCCTGT | |
| | GTTAGCTAACAGGGTACTTGAGTCT | |
| | ATTACCTGGATTTACAGTGGTCTGA | |
| | CAGACAACCAAGCAGCAGAGATGCT | |
| | CTGTATCCTCCGCTTGTGGGGCCAC | |
| | CCTACATTAGAGTCCAGAACAGCTG | |
| | CAGCTGCAGTGCGAAAGCAAATGTG | |
| | CGCGCCAAAACTCATTGACTTCGAC | |
| | ATGATCCAACAAGTATTGGCTTTCT | |
| | TTAAAGGGACAATCATCAATGGATA | |
| | TAGAAGACAAAACTCAGGAGTCTGG | |
| | CCAAGAGTTAAAAAGGATACTATCT | |
| | ATGGATCAACACTCCAACAGTTGCA | |
| | TGCTGACTATGCAGAGATATCACAC | |
| | GAATTAATGCTGAAAGAATACAAGC | |
| | GTCTAGCAATGCTTGAGTTTGAGAA | |
| | GTGTATTGACATAGACCCAGTATCC | |
| | AATTTAAGCATGTTCTTGAAGGACA | |
| | AGGCTATAGCACACACGCGACCAAA | |
| | TTGGCTGGCATCTTTTAAAAGAACT | |
| | TTGTTATCCGATAGACAGCAGCTCT | |
| | TAGCAAAGGATGCAACTTCGACCAA | |
| | TCGTCTGCTGATAGAATTCCTAGAA | |
| | TCTAGCAACTTTGACCCATATCAGG | |
| | AGATGACCTATTTGACAAGTCTTGA | |
| | ATTTCTTAGAGATAATGACGTGGCA | |
| | GTATCATATTCGTTAAAGGAGAAAG | |
| | AAGTTAAGCCCAATGGTAGAATCTT | |
| | CGCAAAGCTTACCAAACGACTCAGA | |
| | AATTGTCAGGTGATGGCAGAGAATA | |
| | TCCTAGCAGACGAAATTGCACCTTT | |
| | TTTCCAAGGGAATGGAGTCATTCAA | |
| | AGCAGCATCTCTCTGACGAAAAGTA | |
| | TGTTAGCAATGAGTCAACTGTCATT | |
| | TAATTGCAACAGATTCTCGATCGGA | |
| | AACCGCAGAGAAGGGATCAAAGAGA | |
| | ATAGGACACGACACCGTGAACGAAA | |
| | GCGAAGAAGGCGAGTAGCTACATAT | |
| | ATCACAACTGACCTGCAGAAGTACT | |
| | GTCTCAATTGGAGGTATCAGACCAT | |
| | CAAGCCTTTTGCCCATGCGATTAAT | |
| | CAGCTGACAGGGCTTGATTTGTTTT | |
| | TTGAGTGGATCCACCTTCGTCTAAT | |
| | GGATACCACTATGTTCGTTGGAGAT | |
| | CCATACAACCCACCCTCTGATCCAA | |
| | CAATTGAAAACCTGGATGATGCACC | |
| | CAATGATGATATCTTTATTGTAAGC | |
| | GGAAGAGGAGGGATCGAGGGATTAT | |
| | GTCAAAAGCTTTGGACTACCATATC | |
| | AATATCCGCAATACAATTAGCAGCC | |
| | ACCCGGTCAAAGTGTAGGGTAGCCT | |
| | GTATGGTGCAAGGTGACAATCAGGT | |
| | GATCGCAGTGACCCGAGAAGTAAAT | |
| | CCAGATGACTCAGAAGATGCGGTCT | |
| | TAGATGAATTACATAAGGCCAGCGA | |
| | CAGATTCTTTGAGGAACTCACTCAC | |
| | GTGAATCATCTGATCGGACATAACC | |
| | TGAAAGATAGAGAGACCATACGCTC | |
| | AGATACTTGTTTTATCTATAGCAAG | |
| | CGAGTATTCAAGGATGGTAAGATAC | |
| | TTTCTCAGGCCCTCAAGAATGCTGC | |
| | AAAGCTCGTCTTAATATCTGGGGAG | |
| | ATTGGGGAGAACACTCCTATGTCAT | |
| | GCGGGAATATTGCTTCTACAGTGTC | |
| | TCGTCTGTGTGAAAATGGGCTGCCC | |
| | AAAGATGCCTGCTATATGATCAATT | |
| | ATATATTAACCTGTATACAATTTTT | |
| | CTTTGACAATGAGTTTTCCATTGTC | |
| | CCCGCTTCTCAGCGTGGATCCACAG | |
| | TTGAATGGGTGGATAACCTTTCATT | |
| | TGTACACGCGTATGCACTGTGGCCA | |
| | GGCCAATTTGGAGGATTGAACAACT | |
| | TACAATATTCTAGATTGTTTACTCG | |
| | CAATATCGGGGACCCATGCACTACT | |
| | GCACTTGCAGAGATTAAGAGATTAG | |
| | AGAGAGCTCAACTAATACCAGGGAA | |
| | GCTAATCAAGAACTTGCTTGCTAGG | |
| | AAGCCAAGCAATGGAACATGGGCGT | |
| | CTCTTTGTAATGATCCTTATTCACT | |
| | CAATATTGAAACAGCACCAAGCCCA | |
| | AATCTCATCCTCAAGAAACATACTC | |
| | AGAGAGTACTATTTGAATCCTGCAC | |
| | CAATCCCCTATTACAAGGGGTTTAT | |
| | AGTGAAGAAAATGATACGGAAGAAG | |
| | CAGAATTAGCAGAATTCTTGCTCAA | |
| | TCAAGAAGCTATACATCCGCGCGTG | |
| | GCACACGTTATAATGGAGGCCAGCG | |
| | CAGTCGGTAGAAAGAAGCAAATTCA | |
| | GGGACTAATCGATACAACTAACACC | |
| | ATCATAAAGATTGCACTTGGGCGGC | |
| | GTCCTCTTGGTGCAAGGAGGTTAAG | |
| | GAAGATAAACAGTTATTCTTCTATG | |
| | CACATGTTGATCTTCCTGGATGATA | |
| | TATTCCTACCTAACCATCCTCCATC | |
| | TCCCTTCGTCTCCTCAGTGATGTGT | |
| | TCTGTTGCCCTAGCGGATTACCTAC | |
| | GTCAGATTACCTGGTTGCCTCTGAC | |
| | AAATGGTAGGAAGATATTAGGTGTA | |
| | AATAATCCAGATACCCTTGAGTTAG | |
| | TATCAGGATCGATGCTGAATCTAAA | |
| | CGGATATTGTGACTTATGTAATAGT | |
| | GGAGATAACCAATTTACGTGGTTCC | |
| | ATCTCCCAGCAGATATAGAGCTAGC | |
| | GGACAGTTCATCATCCAACCCTCCA | |
| | ATGCGTATACCTTATGTGGGATCCA | |
| | AGACCCAGGAAAGGAGAAATGCATC | |
| | AATGGCCAAGATTAGCAACATGTCC | |
| | CCTCATATGAAGGCAGCATTGAGAT | |
| | TGGCGTCTGTGAAGGTAAGGGCTTA | |
| | CGGTGATAATGAGCATAATTGGCAA | |
| | GTTGCATGGCAGCTAGCAAATACTC | |
| | GATGTGCGATATCCCTTGAACATCT | |
| | AAAACTTCTAGCCCCTCTACCAACT | |
| | GCAGGGAACCTTCAGCATCGATTGG | |
| | ATGATAGCATAACCCAGATGACCTT | |
| | TACTCCCGCTTCTCTCTATCGGGTG | |
| | GCACCTTATATCCACATCTCCAATG | |
| | ACTCACAAAGAATGTTTTCTGATGA | |
| | GGGGGTTAAGGAGAGCAACATCATC | |
| | TATCAGCAGATAATGTTATTGGGTC | |
| | TATCAGCTATCGAATCATTGTTCCC | |
| | CTTGACCACTAATCATGTATATGAA | |
| | GAAGTGACACTACACCTTCATACTC | |
| | AATTCAGCTGCTGCCTGAGAGAGGC | |
| | GGCCCTTGCGGTCCCATTTGAGCTC | |
| | CAGGGCAAAGTACCTAGGATTCGTG | |
| | CTGCTGAGGGGAACCAATTCGTGTA | |
| | TGACTCATCCCCACTTTTGGAACCT | |
| | GAGGCTCTTCAACTCGATGTGGCTA | |
| | CTTTCAAGAACTATGAGTTGGACTT | |
| | AGACCATTATTCAACGATAGACTTG | |
| | ATGCATGTACTTGAGGTTACGTGTG | |
| | GAAAGCTAATAGGTCAGTCGGTGAT | |
| | TTCATACAATGAGGACACTTCTATA | |
| | AAGAATGATGCAATTATTGTATACG | |
| | ATAATACCCGGAATTGGATCAGTGA | |
| | GGCCCAAAATTGTGACCTGGTGAAG | |
| | TTATTTGAGTATGCTGCACTAGAAA | |
| | TCTTGCTGGACTGCGCATTCCAAAT | |
| | GTATTATCTAAGGGTTCGCGGATAC | |
| | AAGAACATCCTAATATACATGGCAG | |
| | ACCTAATTCGTAATATGCCCGGTAT | |
| | ATTGCTCTCTAATATTGCTGCCACA | |
| | ATCTCCCATCCCATTATCCATACTA | |
| | GACTATACAATGCAGGGTTGCTGGA | |
| | TCATGGGAGTGCGCACCAACTTGCA | |
| | AGCATTGATTTTATTGAATTATCAG | |
| | CTAATTTATTGGTAACATGTATAGC | |
| | TCGTGTATGTACTACACTTCTATCC | |
| | GGTGAAACCCTGATGCTTGCATTTC | |
| | CATCCGTTCTAGACGAGAATTTGAC | |
| | GGAGAAAATGTTTCTTCTAATCGCT | |
| | CGATACTGCTCTTTGTTAGCGTTGT | |
| | TGTACTCATCTAAGGTTCCTATACC | |
| | AAATATTAGGGGCCTGACTGCCGAA | |
| | GATAAGTGCCGGATGCTCACAAATC | |
| | ATCTCATGAACCTTCCATCTGAATT | |
| | TCGGCTGACCGAAAATCAGGTACGA | |
| | AATGTACTGCAACCAGCACTGACAA | |
| | CTTTCCCAGCAAACCTCTATTATAT | |
| | GTCAAGAAAGAGTCTTAATATCATC | |
| | AGAGAGAGGGAGATAAAGATGCTAT | |
| | TATTCAAATGTTGTTCCCTGCCGGG | |
| | GATGAAGCTACAAGCACGGTGGCAG | |
| | TTAATTTGGGATACGAAAGTAAATG | |
| | ACCCCATTGTTAAGTGGCGACGCAT | |
| | TGAATTCTTATGCGAGCTCGATCTC | |
| | TCTGGTCAGGCAAGGTTTGGAGTCA | |
| | TACTGGATGAATGCATCTCTGATGT | |
| | TGATAAAAACGGACAGGGCATCCTC | |
| | GACTTTGTCCCAATGACTCGATACC | |
| | TATTCAGGGGTGTAGGCCAGGCATC | |
| | CTCATCATGGTATAAAGCTGCCAAT | |
| | TTATTGTCACTTCCTGAAGTGCGCC | |
| | AGGCACGTTTCGGTAACTCATTGTA | |
| | CTTAGCAGAAGGTAGCGGTGCAATA | |
| | ATGAGTCTGTTAGAGCTCCACGTAC | |
| | CACATGAGAAGATTTACTACAATAC | |
| | TCTCTTTTATAACGAGATGAACCCC | |
| | CCGCAAAGACATTTCGGCCCAACGC | |
| | CAACTCAATTCCTTGCATCGGTCGT | |
| | TTACAAGAACCTTCAGGCAGGTATA | |
| | GTCTGCAAAGATGGGTATGTTCAGG | |
| | AGTTCTGCCCTTTATGGAGAGACGT | |
| | TGCCGATGAAAGTGATCTTGCTTCA | |
| | GATAGGTGTGTCTCATTCATTACAT | |
| | CAGAGGTGCCTGGAGGCACTGTATC | |
| | TCTACTCCATTGTGACATAGAAACA | |
| | ACCCTGGAACCAAGCTGGGCTTACT | |
| | TGGAGCAATTAGCCACTAATATCTC | |
| | TCTAATCGGGATGCACGTCCTGCGA | |
| | GAGAATGGAGTGTTCATCATCAAAG | |
| | TACTATACACCCAGAGTTTCTTTTT | |
| | TCATCTATTGCTGGCAATCTTAGCT | |
| | CCTTGTAGTAAAAGGATACGGATCA | |
| | TATCCAATGGATACTCAGTACGGGG | |
| | AGATTTTGAGTGCTACCTAGTCGCG | |
| | ACAATCAGTTATACAGGGGGGCATG | |
| | TCTTCATGCAAGAGGTGATCCGCTC | |
| | TGCCAAGGCGTTAGTTAGAGGGGGC | |
| | GGTAGTATCATGACAAAACAAGATG | |
| | AACAACAATTGAATCTTGCTTTCCA | |
| | GAGGCAGCTCAACAGGATTCGTGGG | |
| | ATACTGGGACAGAGGATATCGATAA | |
| | TGATACGCTACTTGCAGCATACTAT | |
| | TGATATGGCATTGATTGAAGCGGGA | |
| | GGCCAACCTGTAAGACCGAGCAATG | |
| | TTGGAATCAACAAGGCACTCGACTT | |
| | AGGAGATGAGACATATGAGGAAATC | |
| | ATGATACAGCATATTGACACAACAC | |
| | TTAAGACAGCAATCTTCCTAGAACA | |
| | AGAAGAAGAACTGGCAGACACAGTC | |
| | TTTGTGTTAACACCTTATAACCTAA | |
| | CGGCAAGAGGAAAATGTAATACAGT | |
| | ACTTATTGCATGCACTAAACATCTA | |
| | TTTGAAACAACTATATTACAGACTA | |
| | CACGAGACGACATGGATAAGATAGA | |
| | GAAATTGTTGTCCCTTATCTTACAA | |
| | GGTCATATCTCGCTTCAGGATCTCC | |
| | TGCCACTCAAGTCATATCTTAAACG | |
| | TAGCAATTGTCCCAAGTACCTCCTC | |
| | GATTCACTAGGACGTATCAGGCTAA | |
| | AAGAGGTATTTGAACACTCATCCCG | |
| | CATGGTACTAACCAGACCGATGCAA | |
| | AAGATGTATCTCAAATGTCTCGGAA | |
| | ATGCTATTAAGGGATACCTTGCAGT | |
| | GGATGCATCTCATTGCAATTGAATC | |
| | ATGACGCAATCTCTTTTATACATCA | |
| | TACTCGTAATCAATCATAGTTACCA | |
| | TCATTTTTAAGAAAAACAGTAACGA | |
| | TTTATGGTGTCACGTATGTTGCCAA | |
| | ATCTTTGTTTGGT | |
| Newcastle | ACCAAACAGAGAATCCGTGAGTTAC | SEQ ID |
| disease | GATAAAAGGCGAAAGAGCAATTGAA | NO: 13 |
| virus | GTCACACGGGTAGAAGGTGTGAATC | |
| strain | TCGAGTGCGAGCCCGAAGCACAAAC | |
| LaSota, | TCGAGAAAGCCTTCTGCCAACATGT | |
| complete | CCTCCGTATTTGATGAGTACGAACA | |
| genome | GCTCCTCGCGGCTCAGACTCGCCCC | |
| with | AACGGAGCTCATGGAGGGGGAGAAA | |
| modification | AAGGGAGTACCTTAAAAGTAGACGT | |
| in 5408- | CCCGGTATTCACTCTTAACAGTGAT | |
| 5409-5410 | GACCCAGAAGATAGATGGAGCTTTG | |
| nucleotides | TGGTATTCTGCCTCCGGATTGCTGT | |
| resulting in | TAGCGAAGATGCCAACAAACCACTC | |
| L289A | AGGCAAGGTGCTCTCATATCTCTTT | |
| substitution | TATGCTCCCACTCACAGGTAATGAG | |
| | GAACCATGTTGCCCTTGCAGGGAAA | |
| | CAGAATGAAGCCACATTGGCCGTGC | |
| | TTGAGATTGATGGCTTTGCCAACGG | |
| | CACGCCCCAGTTCAATAATAGGAGT | |
| | GGAGTGTCTGAAGAGAGAGCACAGA | |
| | GATTTGCGATGATAGCAGGATCTCT | |
| | CCCTCGGGCATGCAGCAACGGAACC | |
| | CCGTTCGTCACAGCCGGGGCCGAAG | |
| | ATGATGCACCAGAAGACATCACCGA | |
| | TACCCTGGAGAGGATCCTCTCTATC | |
| | CAGGCTCAAGTATGGGTCACAGTAG | |
| | CAAAAGCCATTACTGCGTATGAGAC | |
| | TGCAGATGAGTCGGAAACAAGGCGA | |
| | ATCAATAAGTATATGCAGCAAGGCA | |
| | GGGTCCAAAAGAAATACATCCTCTA | |
| | CCCCGTATGCAGGAGCACAATCCAA | |
| | CTCACGATCAGACAGTCTCTTGCAG | |
| | TCCGCATCTTTTTGGTTAGCGAGCT | |
| | CAAGAGAGGCCGCAACACGGCAGGT | |
| | GGTACCTCTACTTATTATAACCTGG | |
| | TAGGGGACGTAGACTCATACATCAG | |
| | GAATACCGGGCTTACTGCATTCTTC | |
| | TTGACACTCAAGTACGGAATCAACA | |
| | CCAAGACATCAGCCCTTGCACTTAG | |
| | TAGCCTCTCAGGCGACATCCAGAAG | |
| | ATGAAGCAGCTCATGCGTTTGTATC | |
| | GGATGAAAGGAGATAATGCGCCGTA | |
| | CATGACATTACTTGGTGATAGTGAC | |
| | CAGATGAGCTTTGCGCCTGCCGAGT | |
| | ATGCACAACTTTACTCCTTTGCCAT | |
| | GGGTATGGCATCAGTCCTAGATAAA | |
| | GGTACTGGGAAATACCAATTTGCCA | |
| | GGGACTTTATGAGCACATCATTCTG | |
| | GAGACTTGGAGTAGAGTACGCTCAG | |
| | GCTCAGGGAAGTAGCATTAACGAGG | |
| | ATATGGCTGCCGAGCTAAAGCTAAC | |
| | CCCAGCAGCAAGGAGGGGCCTGGCA | |
| | GCTGCTGCCCAACGGGTCTCCGAGG | |
| | AGACCAGCAGCATAGACATGCCTAC | |
| | TCAACAAGTCGGAGTCCTCACTGGG | |
| | CTTAGCGAGGGGGGGTCCCAAGCTC | |
| | TACAAGGCGGATCGAATAGATCGCA | |
| | AGGGCAACCAGAAGCCGGGGATGGG | |
| | GAGACCCAATTCCTGGATCGGATGA | |
| | GAGCGGTAGCAAATAGCATGAGGGA | |
| | GGCGCCAAACTCTGCACAGGGCACT | |
| | CCCCAATCGGGGCCTCCCCCAACTC | |
| | CTGGGCCATCCCAAGATAACGACAC | |
| | CGACTGGGGGTATTGATGGACAAAA | |
| | CCCAGCCTGCTTCCACAAAAACATC | |
| | CCAATGCCCTCACCCGTAGTCGACC | |
| | CCTCGATTTGCGGCTCTATATGACC | |
| | ACACCCTCAAACAAACATCCCCCTC | |
| | TTTCCTCCCTCCCCCTGCTGTACAA | |
| | CTCCGCACGCCCTAGATACCACAGG | |
| | CACAATGCGGCTCACTAACAATCAA | |
| | AACAGAGCCGAGGGAATTAGAAAAA | |
| | AGTACGGGTAGAAGAGGGATATTCA | |
| | GAGATCAGGGCAAGTCTCCCGAGTC | |
| | TCTGCTCTCTCCTCTACCTGATAGA | |
| | CCAGGACAAACATGGCCACCTTTAC | |
| | AGATGCAGAGATCGACGAGCTATTT | |
| | GAGACAAGTGGAACTGTCATTGACA | |
| | ACATAATTACAGCCCAGGGTAAACC | |
| | AGCAGAGACTGTTGGAAGGAGTGCA | |
| | ATCCCACAAGGCAAGACCAAGGTGC | |
| | TGAGCGCAGCATGGGAGAAGCATGG | |
| | GAGCATCCAGCCACCGGCCAGTCAA | |
| | GACAACCCCGATCGACAGGACAGAT | |
| | CTGACAAACAACCATCCACACCCGA | |
| | GCAAACGACCCCGCATGACAGCCCG | |
| | CCGGCCACATCCGCCGACCAGCCCC | |
| | CCACCCAGGCCACAGACGAAGCCGT | |
| | CGACACACAGCTCAGGACCGGAGCA | |
| | AGCAACTCTCTGCTGTTGATGCTTG | |
| | ACAAGCTCAGCAATAAATCGTCCAA | |
| | TGCTAAAAAGGGCCCATGGTCGAGC | |
| | CCCCAAGAGGGGAATCACCAACGTC | |
| | CGACTCAACAGCAGGGGAGTCAACC | |
| | CAGTCGCGGAAACAGTCAGGAAAGA | |
| | CCGCAGAACCAAGTCAAGGCCGCCC | |
| | CTGGAAACCAGGGCACAGACGTGAA | |
| | CACAGCATATCATGGACAATGGGAG | |
| | GAGTCACAACTATCAGCTGGTGCAA | |
| | CCCCTCATGCTCTCCGATCAAGGCA | |
| | GAGCCAAGACAATACCCTTGTATCT | |
| | GCGGATCATGTCCAGCCACCTGTAG | |
| | ACTTTGTGCAAGCGATGATGTCTAT | |
| | GATGGAGGCGATATCACAGAGAGTA | |
| | AGTAAGGTCGACTATCAGCTAGATC | |
| | TTGTCTTGAAACAGACATCCTCCAT | |
| | CCCTATGATGCGGTCCGAAATCCAA | |
| | CAGCTGAAAACATCTGTTGCAGTCA | |
| | TGGAAGCCAACTTGGGAATGATGAA | |
| | GATTCTGGATCCCGGTTGTGCCAAC | |
| | ATTTCATCTCTGAGTGATCTACGGG | |
| | CAGTTGCCCGATCTCACCCGGTTTT | |
| | AGTTTCAGGCCCTGGAGACCCCTCT | |
| | CCCTATGTGACACAAGGAGGCGAAA | |
| | TGGCACTTAATAAACTTTCGCAACC | |
| | AGTGCCACATCCATCTGAATTGATT | |
| | AAACCCGCCACTGCATGCGGGCCTG | |
| | ATATAGGAGTGGAAAAGGACACTGT | |
| | CCGTGCATTGATCATGTCACGCCCA | |
| | ATGCACCCGAGTTCTTCAGCCAAGC | |
| | TCCTAAGCAAGTTAGATGCAGCCGG | |
| | GTCGATCGAGGAAATCAGGAAAATC | |
| | AAGCGCCTTGCTCTAAATGGCTAAT | |
| | TACTACTGCCACACGTAGCGGGTCC | |
| | CTGTCCACTCGGCATCACACGGAAT | |
| | CTGCACCGAGTTCCCCCCCGCAGAC | |
| | CCAAGGTCCAACTCTCCAAGCGGCA | |
| | ATCCTCTCTCGCTTCCTCAGCCCCA | |
| | CTGAATGATCGCGTAACCGTAATTA | |
| | ATCTAGCTACATTTAAGATTAAGAA | |
| | AAAATACGGGTAGAATTGGAGTGCC | |
| | CCAATTGTGCCAAGATGGACTCATC | |
| | TAGGACAATTGGGCTGTACTTTGAT | |
| | TCTGCCCATTCTTCTAGCAACCTGT | |
| | TAGCATTTCCGATCGTCCTACAAGA | |
| | CACAGGAGATGGGAAGAAGCAAATC | |
| | GCCCCGCAATATAGGATCCAGCGCC | |
| | TTGACTTGTGGACTGATAGTAAGGA | |
| | GGACTCAGTATTCATCACCACCTAT | |
| | GGATTCATCTTTCAAGTTGGGAATG | |
| | AAGAAGCCACTGTCGGCATGATCGA | |
| | TGATAAACCCAAGCGCGAGTTACTT | |
| | TCCGCTGCGATGCTCTGCCTAGGAA | |
| | GCGTCCCAAATACCGGAGACCTTAT | |
| | TGAGCTGGCAAGGGCCTGTCTCACT | |
| | ATGATAGTCACATGCAAGAAGAGTG | |
| | CAACTAATACTGAGAGAATGGTTTT | |
| | CTCAGTAGTGCAGGCACCCCAAGTG | |
| | CTGCAAAGCTGTAGGGTTGTGGCAA | |
| | ACAAATACTCATCAGTGAATGCAGT | |
| | CAAGCACGTGAAAGCGCCAGAGAAG | |
| | ATTCCCGGGAGTGGAACCCTAGAAT | |
| | ACAAGGTGAACTTTGTCTCCTTGAC | |
| | TGTGGTACCGAAGAAGGATGTCTAC | |
| | AAGATCCCTGCTGCAGTATTGAAGG | |
| | TTTCTGGCTCGAGTCTGTACAATCT | |
| | TGCGCTCAATGTCACTATTAATGTG | |
| | GAGGTAGACCCGAGGAGTCCTTTGG | |
| | TTAAATCTCTGTCTAAGTCTGACAG | |
| | CGGATACTATGCTAACCTCTTCTTG | |
| | CATATTGGACTTATGACCACCGTAG | |
| | ATAGGAAGGGGAAGAAAGTGACATT | |
| | TGACAAGCTGGAAAAGAAAATAAGG | |
| | AGCCTTGATCTATCTGTCGGGCTCA | |
| | GTGATGTGCTCGGGCCTTCCGTGTT | |
| | GGTAAAAGCAAGAGGTGCACGGACT | |
| | AAGCTTTTGGCACCTTTCTTCTCTA | |
| | GCAGTGGGACAGCCTGCTATCCCAT | |
| | AGCAAATGCTTCTCCTCAGGTGGCC | |
| | AAGATACTCTGGAGTCAAACCGCGT | |
| | GCCTGCGGAGCGTTAAAATCATTAT | |
| | CCAAGCAGGTACCCAACGCGCTGTC | |
| | GCAGTGACCGCCGACCACGAGGTTA | |
| | CCTCTACTAAGCTGGAGAAGGGGCA | |
| | CACCCTTGCCAAATACAATCCTTTT | |
| | AAGAAATAAGCTGCGTCTCTGAGAT | |
| | TGCGCTCCGCCCACTCACCCAGATC | |
| | ATCATGACACAAAAAACTAATCTGT | |
| | CTTGATTATTTACAGTTAGTTTACC | |
| | TGTCTATCAAGTTAGAAAAAACACG | |
| | GGTAGAAGATTCTGGATCCCGGTTG | |
| | GCGCCCTCCAGGTGCAAGATGGGCT | |
| | CCAGACCTTCTACCAAGAACCCAGC | |
| | ACCTATGATGCTGACTATCCGGGTT | |
| | GCGCTGGTACTGAGTTGCATCTGTC | |
| | CGGCAAACTCCATTGATGGCAGGCC | |
| | TCTTGCAGCTGCAGGAATTGTGGTT | |
| | ACAGGAGACAAAGCCGTCAACATAT | |
| | ACACCTCATCCCAGACAGGATCAAT | |
| | CATAGTTAAGCTCCTCCCGAATCTG | |
| | CCCAAGGATAAGGAGGCATGTGCGA | |
| | AAGCCCCCTTGGATGCATACAACAG | |
| | GACATTGACCACTTTGCTCACCCCC | |
| | CTTGGTGACTCTATCCGTAGGATAC | |
| | AAGAGTCTGTGACTACATCTGGAGG | |
| | GGGGAGACAGGGGCGCCTTATAGGT | |
| | GCCATTATTGGCGGTGTGGCTCTTG | |
| | GGGTTGCAACTGCCGCACAAATAAC | |
| | AGCGGCCGCAGCTCTGATACAAGCC | |
| | AAACAAAATGCTGCCAACATCCTCC | |
| | GACTTAAAGAGAGCATTGCCGCAAC | |
| | CAATGAGGCTGTGCATGAGGTCACT | |
| | GACGGATTATCGCAACTAGCAGTGG | |
| | CAGTTGGGAAGATGCAGCAGTTTGT | |
| | TAATGACCAATTTAATAAAACAGCT | |
| | CAGGAATTAGACTGCATCAAAATTG | |
| | CACAGCAAGTTGGTGTAGAGCTCAA | |
| | CCTGTACCTAACCGAATTGACTACA | |
| | GTATTCGGACCACAAATCACTTCAC | |
| | CCGCTTTAAACAAGCTGACTATTCA | |
| | GGCACTTTACAATCTAGCTGGTGGA | |
| | AATATGGATTACTTATTGACTAAGT | |
| | TAGGTGTAGGGAACAATCAACTCAG | |
| | CTCATTAATCGGTAGCGGCTTAATC | |
| | ACCGGTAACCCTATTCTATACGACT | |
| | CACAGACTCAACTCTTGGGTATACA | |
| | GGTAACTGCCCCTTCAGTCGGGAAC | |
| | CTAAATAATATGCGTGCCACCTACT | |
| | TGGAAACCTTATCCGTAAGCACAAC | |
| | CAGGGGATTTGCCTCGGCACTTGTC | |
| | CCAAAAGTGGTGACACAGGTCGGTT | |
| | CTGTGATAGAAGAACTTGACACCTC | |
| | ATACTGTATAGAAACTGACTTAGAT | |
| | TTATATTGTACAAGAATAGTAACGT | |
| | TCCCTATGTCCCCTGGTATTTATTC | |
| | CTGCTTGAGCGGCAATACGTCGGCC | |
| | TGTATGTACTCAAAGACCGAAGGCG | |
| | CACTTACTACACCATACATGACTAT | |
| | CAAAGGTTCAGTCATCGCCAACTGC | |
| | AAGATGACAACATGTAGATGTGTAA | |
| | ACCCCCCGGGTATCATATCGCAAAA | |
| | CTATGGAGAAGCCGTGTCTCTAATA | |
| | GATAAACAATCATGCAATGTTTTAT | |
| | CCTTAGGCGGGATAACTTTAAGGCT | |
| | CAGTGGGGAATTCGATGTAACTTAT | |
| | CAGAAGAATATCTCAATACAAGATT | |
| | CTCAAGTAATAATAACAGGCAATCT | |
| | TGATATCTCAACTGAGCTTGGGAAT | |
| | GTCAACAACTCGATCAGTAATGCTT | |
| | TGAATAAGTTAGAGGAAAGCAACAG | |
| | AAAACTAGACAAAGTCAATGTCAAA | |
| | CTGACTAGCACATCTGCCCTCATTA | |
| | CCTATATCGTTTTGACTATCATATC | |
| | TCTTGTTTTTGGTATACTTAGCCTG | |
| | ATTCTAGCATGCTACCTAATGTACA | |
| | AGCAAAAGGCGCAACAAAAGACCTT | |
| | ATTATGGCTTGGGAATAATACTCTA | |
| | GATCAGATGAGAGCCACTACAAAAA | |
| | TGTGAACACAGATGAGGAACGAAGG | |
| | TTTCCCTAATAGTAATTTGTGTGAA | |
| | AGTTCTGGTAGTCTGTCAGTTCAGA | |
| | GAGTTAAGAAAAAACTACCGGTTGT | |
| | AGATGACCAAAGGACGATATACGGG | |
| | TAGAACGGTAAGAGAGGCCGCCCCT | |
| | CAATTGCGAGCCAGGCTTCACAACC | |
| | TCCGTTCTACCGCTTCACCGACAAC | |
| | AGTCCTCAATCATGGACCGCGCCGT | |
| | TAGCCAAGTTGCGTTAGAGAATGAT | |
| | GAAAGAGAGGCAAAAAATACATGGC | |
| | GCTTGATATTCCGGATTGCAATCTT | |
| | ATTCTTAACAGTAGTGACCTTGGCT | |
| | ATATCTGTAGCCTCCCTTTTATATA | |
| | GCATGGGGGCTAGCACACCTAGCGA | |
| | TCTTGTAGGCATACCGACTAGGATT | |
| | TCCAGGGCAGAAGAAAAGATTACAT | |
| | CTACACTTGGTTCCAATCAAGATGT | |
| | AGTAGATAGGATATATAAGCAAGTG | |
| | GCCCTTGAGTCTCCGTTGGCATTGT | |
| | TAAAAACTGAGACCACAATTATGAA | |
| | CGCAATAACATCTCTCTCTTATCAG | |
| | ATTAATGGAGCTGCAAACAACAGTG | |
| | GGTGGGGGGCACCTATCCATGACCC | |
| | AGATTATATAGGGGGGATAGGCAAA | |
| | GAACTCATTGTAGATGATGCTAGTG | |
| | ATGTCACATCATTCTATCCCTCTGC | |
| | ATTTCAAGAACATCTGAATTTTATC | |
| | CCGGCGCCTACTACAGGATCAGGTT | |
| | GCACTCGAATACCCTCATTTGACAT | |
| | GAGTGCTACCCATTACTGCTACACC | |
| | CATAATGTAATATTGTCTGGATGCA | |
| | GAGATCACTCACATTCATATCAGTA | |
| | TTTAGCACTTGGTGTGCTCCGGACA | |
| | TCTGCAACAGGGAGGGTATTCTTTT | |
| | CTACTCTGCGTTCCATCAACCTGGA | |
| | CGACACCCAAAATCGGAAGTCTTGC | |
| | AGTGTGAGTGCAACTCCCCTGGGTT | |
| | GTGATATGCTGTGCTCGAAAGTCAC | |
| | GGAGACAGAGGAAGAAGATTATAAC | |
| | TCAGCTGTCCCTACGCGGATGGTAC | |
| | ATGGGAGGTTAGGGTTCGACGGCCA | |
| | GTACCACGAAAAGGACCTAGATGTC | |
| | ACAACATTATTCGGGGACTGGGTGG | |
| | CCAACTACCCAGGAGTAGGGGGTGG | |
| | ATCTTTTATTGACAGCCGCGTATGG | |
| | TTCTCAGTCTACGGAGGGTTAAAAC | |
| | CCAATTCACCCAGTGACACTGTACA | |
| | GGAAGGGAAATATGTGATATACAAG | |
| | CGATACAATGACACATGCCCAGATG | |
| | AGCAAGACTACCAGATTCGAATGGC | |
| | CAAGTCTTCGTATAAGCCTGGACGG | |
| | TTTGGTGGGAAACGCATACAGCAGG | |
| | CTATCTTATCTATCAAGGTGTCAAC | |
| | ATCCTTAGGCGAAGACCCGGTACTG | |
| | ACTGTACCGCCCAACACAGTCACAC | |
| | TCATGGGGGCCGAAGGCAGAATTCT | |
| | CACAGTAGGGACATCTCATTTCTTG | |
| | TATCAACGAGGGTCATCATACTTCT | |
| | CTCCCGCGTTATTATATCCTATGAC | |
| | AGTCAGCAACAAAACAGCCACTCTT | |
| | CATAGTCCTTATACATTCAATGCCT | |
| | TCACTCGGCCAGGTAGTATCCCTTG | |
| | CCAGGCTTCAGCAAGATGCCCCAAC | |
| | CCGTGTGTTACTGGAGTCTATACAG | |
| | ATCCATATCCCCTAATCTTCTATAG | |
| | AAACCACACCTTGCGAGGGGTATTC | |
| | GGGACAATGCTTGATGGTGTACAAG | |
| | CAAGACTTAACCCTGCGTCTGCAGT | |
| | ATTCGATAGCACATCCCGCAGTCGC | |
| | ATTACTCGAGTGAGTTCAAGCAGTA | |
| | CCAAAGCAGCATACACAACATCAAC | |
| | TTGTTTTAAAGTGGTCAAGACTAAT | |
| | AAGACCTATTGTCTCAGCATTGCTG | |
| | AAATATCTAATACTCTCTTCGGAGA | |
| | ATTCAGAATCGTCCCGTTACTAGTT | |
| | GAGATCCTCAAAGATGACGGGGTTA | |
| | GAGAAGCCAGGTCTGGCTAGTTGAG | |
| | TCAATTATAAAGGAGTTGGAAAGAT | |
| | GGCATTGTATCACCTATCTTCCACG | |
| | ACATCAAGAATCAAACCGAATGCCG | |
| | GCGCGTGCTCGAATTCCATGTTGCC | |
| | AGTTGACCACAATCAGCCAGTGCTC | |
| | ATGCGATCAGATTAAGCCTTGTCAA | |
| | TAGTCTCTTGATTAAGAAAAAATGT | |
| | AAGTGGCAATGAGATACAAGGCAAA | |
| | ACAGCTCATGGTAAATAATACGGGT | |
| | AGGACATGGCGAGCTCCGGTCCTGA | |
| | AAGGGCAGAGCATCAGATTATCCTA | |
| | CCAGAGTCACACCTGTCTTCACCAT | |
| | TGGTCAAGCACAAACTACTCTATTA | |
| | CTGGAAATTAACTGGGCTACCGCTT | |
| | CCTGATGAATGTGACTTCGACCACC | |
| | TCATTCTCAGTCGACAATGGAAAAA | |
| | AATACTTGAATCGGCCTCTCCTGAT | |
| | ACTGAGAGAATGATAAAACTCGGAA | |
| | GGGCAGTACACCAAACTCTTAACCA | |
| | CAATTCCAGAATAACCGGAGTGCTC | |
| | CACCCCAGGTGTTTAGAAGAACTGG | |
| | CTAATATTGAGGTCCCAGATTCAAC | |
| | CAACAAATTTCGGAAGATTGAGAAG | |
| | AAGATCCAAATTCACAACACGAGAT | |
| | ATGGAGAACTGTTCACAAGGCTGTG | |
| | TACGCATATAGAGAAGAAACTGCTG | |
| | GGGTCATCTTGGTCTAACAATGTCC | |
| | CCCGGTCAGAGGAGTTCAGCAGCAT | |
| | TCGTACGGATCCGGCATTCTGGTTT | |
| | CACTCAAAATGGTCCACAGCCAAGT | |
| | TTGCATGGCTCCATATAAAACAGAT | |
| | CCAGAGGCATCTGATGGTGGCAGCT | |
| | AGGACAAGGTCTGCGGCCAACAAAT | |
| | TGGTGATGCTAACCCATAAGGTAGG | |
| | CCAAGTCTTTGTCACTCCTGAACTT | |
| | GTCGTTGTGACGCATACGAATGAGA | |
| | ACAAGTTCACATGTCTTACCCAGGA | |
| | ACTTGTATTGATGTATGCAGATATG | |
| | ATGGAGGGCAGAGATATGGTCAACA | |
| | TAATATCAACCACGGCGGTGCATCT | |
| | CAGAAGCTTATCAGAGAAAATTGAT | |
| | GACATTTTGCGGTTAATAGACGCTC | |
| | TGGCAAAAGACTTGGGTAATCAAGT | |
| | CTACGATGTTGTATCACTAATGGAG | |
| | GGATTTGCATACGGAGCTGTCCAGC | |
| | TACTCGAGCCGTCAGGTACATTTGC | |
| | AGGAGATTTCTTCGCATTCAACCTG | |
| | CAGGAGCTTAAAGACATTCTAATTG | |
| | GCCTCCTCCCCAATGATATAGCAGA | |
| | ATCCGTGACTCATGCAATCGCTACT | |
| | GTATTCTCTGGTTTAGAACAGAATC | |
| | AAGCAGCTGAGATGTTGTGTCTGTT | |
| | GCGTCTGTGGGGTCACCCACTGCTT | |
| | GAGTCCCGTATTGCAGCAAAGGCAG | |
| | TCAGGAGCCAAATGTGCGCACCGAA | |
| | AATGGTAGACTTTGATATGATCCTT | |
| | CAGGTACTGTCTTTCTTCAAGGGAA | |
| | CAATCATCAACGGGTACAGAAAGAA | |
| | GAATGCAGGTGTGTGGCCGCGAGTC | |
| | AAAGTGGATACAATATATGGGAAGG | |
| | TCATTGGGCAACTACATGCAGATTC | |
| | AGCAGAGATTTCACACGATATCATG | |
| | TTGAGAGAGTATAAGAGTTTATCTG | |
| | CACTTGAATTTGAGCCATGTATAGA | |
| | ATATGACCCTGTCACCAACCTGAGC | |
| | ATGTTCCTAAAAGACAAGGCAATCG | |
| | CACACCCCAACGATAATTGGCTTGC | |
| | CTCGTTTAGGCGGAACCTTCTCTCC | |
| | GAAGACCAGAAGAAACATGTAAAAG | |
| | AAGCAACTTCGACTAATCGCCTCTT | |
| | GATAGAGTTTTTAGAGTCAAATGAT | |
| | TTTGATCCATATAAAGAGATGGAAT | |
| | ATCTGACGACCCTTGAGTACCTTAG | |
| | AGATGACAATGTGGCAGTATCATAC | |
| | TCGCTCAAGGAGAAGGAAGTGAAAG | |
| | TTAATGGACGGATCTTCGCTAAGCT | |
| | GACAAAGAAGTTAAGGAACTGTCAG | |
| | GTGATGGCGGAAGGGATCCTAGCCG | |
| | ATCAGATTGCACCTTTCTTTCAGGG | |
| | AAATGGAGTCATTCAGGATAGCATA | |
| | TCCTTGACCAAGAGTATGCTAGCGA | |
| | TGAGTCAACTGTCTTTTAACAGCAA | |
| | TAAGAAACGTATCACTGACTGTAAA | |
| | GAAAGAGTATCTTCAAACCGCAATC | |
| | ATGATCCGAAAAGCAAGAACCGTCG | |
| | GAGAGTTGCAACCTTCATAACAACT | |
| | GACCTGCAAAAGTACTGTCTTAATT | |
| | GGAGATATCAGACAATCAAATTGTT | |
| | CGCTCATGCCATCAATCAGTTGATG | |
| | GGCCTACCTCACTTCTTCGAATGGA | |
| | TTCACCTAAGACTGATGGACACTAC | |
| | GATGTTCGTAGGAGACCCTTTCAAT | |
| | CCTCCAAGTGACCCTACTGACTGTG | |
| | ACCTCTCAAGAGTCCCTAATGATGA | |
| | CATATATATTGTCAGTGCCAGAGGG | |
| | GGTATCGAAGGATTATGCCAGAAGC | |
| | TATGGACAATGATCTCAATTGCTGC | |
| | AATCCAACTTGCTGCAGCTAGATCG | |
| | CATTGTCGTGTTGCCTGTATGGTAC | |
| | AGGGTGATAATCAAGTAATAGCAGT | |
| | AACGAGAGAGGTAAGATCAGACGAC | |
| | TCTCCGGAGATGGTGTTGACACAGT | |
| | TGCATCAAGCCAGTGATAATTTCTT | |
| | CAAGGAATTAATTCATGTCAATCAT | |
| | TTGATTGGCCATAATTTGAAGGATC | |
| | GTGAAACCATCAGGTCAGACACATT | |
| | CTTCATATACAGCAAACGAATCTTC | |
| | AAAGATGGAGCAATCCTCAGTCAAG | |
| | TCCTCAAAAATTCATCTAAATTAGT | |
| | GCTAGTGTCAGGTGATCTCAGTGAA | |
| | AACACCGTAATGTCCTGTGCCAACA | |
| | TTGCCTCTACTGTAGCACGGCTATG | |
| | CGAGAACGGGCTTCCCAAAGACTTC | |
| | TGTTACTATTTAAACTATATAATGA | |
| | GTTGTGTGCAGACATACTTTGACTC | |
| | TGAGTTCTCCATCACCAACAATTCG | |
| | CACCCCGATCTTAATCAGTCGTGGA | |
| | TTGAAGACATCTCTTTTGTGCACTC | |
| | ATATGTTCTGACTCCTGCCCAATTA | |
| | GGGGGACTGAGTAACCTTCAATACT | |
| | CAAGGCTCTACACTAGAAATATCGG | |
| | TGACCCGGGGACTACTGCTTTTGCA | |
| | GAGATCAAGCGACTAGAAGCAGTGG | |
| | GATTACTGAGTCCTAACATTATGAC | |
| | TAATATCTTAACTAGGCCGCCTGGG | |
| | AATGGAGATTGGGCCAGTCTGTGCA | |
| | ACGACCCATACTCTTTCAATTTTGA | |
| | GACTGTTGCAAGCCCAAATATTGTT | |
| | CTTAAGAAACATACGCAAAGAGTCC | |
| | TATTTGAAACTTGTTCAAATCCCTT | |
| | ATTGTCTGGAGTGCACACAGAGGAT | |
| | AATGAGGCAGAAGAGAAGGCATTGG | |
| | CTGAATTCTTGCTTAATCAAGAGGT | |
| | GATTCATCCCCGCGTTGCGCATGCC | |
| | ATCATGGAGGCAAGCTCTGTAGGTA | |
| | GGAGAAAGCAAATTCAAGGGCTTGT | |
| | TGACACAACAAACACCGTAATTAAG | |
| | ATTGCGCTTACTAGGAGGCCATTAG | |
| | GCATCAAGAGGCTGATGCGGATAGT | |
| | CAATTATTCTAGCATGCATGCAATG | |
| | CTGTTTAGAGACGATGTTTTTTCCT | |
| | CCAGTAGATCCAACCACCCCTTAGT | |
| | CTCTTCTAATATGTGTTCTCTGACA | |
| | CTGGCAGACTATGCACGGAATAGAA | |
| | GCTGGTCACCTTTGACGGGAGGCAG | |
| | GAAAATACTGGGTGTATCTAATCCT | |
| | GATACGATAGAACTCGTAGAGGGTG | |
| | AGATTCTTAGTGTAAGCGGAGGGTG | |
| | TACAAGATGTGACAGCGGAGATGAA | |
| | CAATTTACTTGGTTCCATCTTCCAA | |
| | GCAATATAGAATTGACCGATGACAC | |
| | CAGCAAGAATCCTCCGATGAGGGTA | |
| | CCATATCTCGGGTCAAAGACACAGG | |
| | AGAGGAGAGCTGCCTCACTTGCAAA | |
| | AATAGCTCATATGTCGCCACATGTA | |
| | AAGGCTGCCCTAAGGGCATCATCCG | |
| | TGTTGATCTGGGCTTATGGGGATAA | |
| | TGAAGTAAATTGGACTGCTGCTCTT | |
| | ACGATTGCAAAATCTCGGTGTAATG | |
| | TAAACTTAGAGTATCTTCGGTTACT | |
| | GTCCCCTTTACCCACGGCTGGGAAT | |
| | CTTCAACATAGACTAGATGATGGTA | |
| | TAACTCAGATGACATTCACCCCTGC | |
| | ATCTCTCTACAGGGTGTCACCTTAC | |
| | ATTCACATATCCAATGATTCTCAAA | |
| | GGCTGTTCACTGAAGAAGGAGTCAA | |
| | AGAGGGGAATGTGGTTTACCAACAG | |
| | ATCATGCTCTTGGGTTTATCTCTAA | |
| | TCGAATCGATCTTTCCAATGACAAC | |
| | AACCAGGACATATGATGAGATCACA | |
| | CTGCACCTACATAGTAAATTTAGTT | |
| | GCTGTATCAGAGAAGCACCTGTTGC | |
| | GGTTCCTTTCGAGCTACTTGGGGTG | |
| | GTACCGGAACTGAGGACAGTGACCT | |
| | CAAATAAGTTTATGTATGATCCTAG | |
| | CCCTGTATCGGAGGGAGACTTTGCG | |
| | AGACTTGACTTAGCTATCTTCAAGA | |
| | GTTATGAGCTCAATCTGGAGTCATA | |
| | TCCCACGATAGAGCTAATGAACATT | |
| | CTTTCAATATCCAGCGGGAAGTTGA | |
| | TTGGCCAGTCTGTGGTTTCTTATGA | |
| | TGAAGATACCTCCATAAAGAATGAC | |
| | GCCATAATAGTGTATGACAATACCC | |
| | GAAATTGGATCAGTGAAGCTCAGAA | |
| | TTCAGATGTGGTCCGCCTATTTGAA | |
| | TATGCAGCACTTGAAGTGCTCCTCG | |
| | ACTGTTCTTACCAACTCTATTACCT | |
| | GAGAGTAAGAGGCCTAGACAATATT | |
| | GTCTTATATATGGGTGATTTATACA | |
| | AGAATATGCCAGGAATTCTACTTTC | |
| | CAACATTGCAGCTACAATATCTCAT | |
| | CCCGTCATTCATTCAAGGTTACATG | |
| | CAGTGGGCCTGGTCAACCATGACGG | |
| | ATCACACCAACTTGCAGATACGGAT | |
| | TTTATCGAAATGTCTGCAAAACTAT | |
| | TAGTATCTTGCACCCGACGTGTGAT | |
| | CTCCGGCTTATATTCAGGAAATAAG | |
| | TATGATCTGCTGTTCCCATCTGTCT | |
| | TAGATGATAACCTGAATGAGAAGAT | |
| | GCTTCAGCTGATATCCCGGTTATGC | |
| | TGTCTGTACACGGTACTCTTTGCTA | |
| | CAACAAGAGAAATCCCGAAAATAAG | |
| | AGGCTTAACTGCAGAAGAGAAATGT | |
| | TCAATACTCACTGAGTATTTACTGT | |
| | CGGATGCTGTGAAACCATTACTTAG | |
| | CCCCGATCAAGTGAGCTCTATCATG | |
| | TCTCCTAACATAATTACATTCCCAG | |
| | CTAATCTGTACTACATGTCTCGGAA | |
| | GAGCCTCAATTTGATCAGGGAAAGG | |
| | GAGGACAGGGATACTATCCTGGCGT | |
| | TGTTGTTCCCCCAAGAGCCATTATT | |
| | AGAGTTCCCTTCTGTGCAAGATATT | |
| | GGTGCTCGAGTGAAAGATCCATTCA | |
| | CCCGACAACCTGCGGCATTTTTGCA | |
| | AGAGTTAGATTTGAGTGCTCCAGCA | |
| | AGGTATGACGCATTCACACTTAGTC | |
| | AGATTCATCCTGAACTCACATCTCC | |
| | AAATCCGGAGGAAGACCACTTAGTA | |
| | CGATACTTGTTCAGAGGGATAGGGA | |
| | CTGCATCTTCCTCTTGGTATAAGGC | |
| | ATCTCATCTCCTTTCTGTACCCGAG | |
| | GTAAGATGTGCAAGACACGGGAACT | |
| | CCTTATACTTAGCTGAAGGGAGCGG | |
| | AGCCATCATGAGTCTTCTCGAACTG | |
| | CATGTACCACATGAAACTATCTATT | |
| | ACAATACGCTCTTTTCAAATGAGAT | |
| | GAACCCCCCGCAACGACATTTCGGG | |
| | CCGACCCCAACTCAGTTTTTGAATT | |
| | CGGTTGTTTATAGGAATCTACAGGC | |
| | GGAGGTAACATGCAAAGATGGATTT | |
| | GTCCAAGAGTTCCGTCCATTATGGA | |
| | GAGAAAATACAGAGGAAAGTGACCT | |
| | GACCTCAGATAAAGCAGTGGGGTAT | |
| | ATTACATCTGCAGTGCCCTACAGAT | |
| | CTGTATCATTGCTGCATTGTGACAT | |
| | TGAAATTCCTCCAGGGTCCAATCAA | |
| | AGCTTACTAGATCAACTAGCTATCA | |
| | ATTTATCTCTGATTGCCATGCATTC | |
| | TGTAAGGGAGGGCGGGGTAGTAATC | |
| | ATCAAAGTGTTGTATGCAATGGGAT | |
| | ACTACTTTCATCTACTCATGAACTT | |
| | GTTTGCTCCGTGTTCCACAAAAGGA | |
| | TATATTCTCTCTAATGGTTATGCAT | |
| | GTCGAGGAGATATGGAGTGTTACCT | |
| | GGTATTTGTCATGGGTTACCTGGGC | |
| | GGGCCTACATTTGTACATGAGGTGG | |
| | TGAGGATGGCAAAAACTCTGGTGCA | |
| | GCGGCACGGTACGCTTTTGTCTAAA | |
| | TCAGATGAGATCACACTGACCAGGT | |
| | TATTCACCTCACAGCGGCAGCGTGT | |
| | GACAGACATCCTATCCAGTCCTTTA | |
| | CCAAGATTAATAAAGTACTTGAGGA | |
| | AGAAATTGACACTGCGCTGATTGAA | |
| | GCCGGGGGACAGCCCGTCCGTCCAT | |
| | TCTGTGCGGAGAGTCTGGTGAGCAC | |
| | GCTAGCGAACATAACTCAGATAACC | |
| | CAGATCATCGCTAGCCACATTGACA | |
| | CAGTTATCCGGTCTGTGATATATAT | |
| | GGAAGCTGAGGGTGATCTCGCTGAC | |
| | ACAGTATTTCTATTTACCCCTTACA | |
| | ATCTCTCTACTGACGGGAAAAAGAG | |
| | GACATCACTTAAACAGTGCACGAGA | |
| | CAGATCCTAGAGGTTACAATACTAG | |
| | GTCTTAGAGTCGAAAATCTCAATAA | |
| | AATAGGCGATATAATCAGCCTAGTG | |
| | CTTAAAGGCATGATCTCCATGGAGG | |
| | ACCTTATCCCACTAAGGACATACTT | |
| | GAAGCATAGTACCTGCCCTAAATAT | |
| | TTGAAGGCTGTCCTAGGTATTACCA | |
| | AACTCAAAGAAATGTTTACAGACAC | |
| | TTCTGTACTGTACTTGACTCGTGCT | |
| | CAACAAAAATTCTACATGAAAACTA | |
| | TAGGCAATGCAGTCAAAGGATATTA | |
| | CAGTAACTGTGACTCTTAACGAAAA | |
| | TCACATATTAATAGGCTCCTTTTTT | |
| | GGCCAATTGTATTCTTGTTGATTTA | |
| | ATCATATTATGTTAGAAAAAAGTTG | |
| | AACCCTGACTCCTTAGGACTCGAAT | |
| | TCGAACTCAAATAAATGTCTTAAAA | |
| | AAAGGTTGCGCACAATTATTCTTGA | |
| | GTGTAGTCTCGTCATTCACCAAATC | |
| | TTTGTTTGGT | |
| APMV- | ACGAAAAAGAAGAATAAAAGGCAGA | SEQ ID |
| 4_hIL12_ | AGCCTTTTAAAAGGAACCCTGGGCT | NO: 14 |
| SCC_AGS | GTCGTAGGTGTGGGAAGGTTGTATT | |
| | CCGAGTGCGCCTCCGAGGCATCTAC | |
| | TCTACACCTATCACAATGGCTGGTG | |
| | TCTTCTCCCAGTATGAGAGGTTTGT | |
| | GGACAATCAATCCCAAGTGTCAAGG | |
| | AAGGATCATCGGTCCTTAGCAGGAG | |
| | GATGCCTTAAAGTTAACATCCCTAT | |
| | GCTTGTCACTGCATCTGAAGACCCC | |
| | ACCACTCGTTGGCAACTAGCATGCT | |
| | TATCTCTAAGGCTCCTGATCTCCAA | |
| | CTCATCAACCAGTGCTATCCGTCAG | |
| | GGGGCAATACTGACTCTCATGTCAT | |
| | TACCATCACAAAACATGAGAGCAAC | |
| | AGCAGCTATTGCTGGTTCCACAAAT | |
| | GCAGCTGTTATCAACACCATGGAAG | |
| | TCTTAAGTGTCAACGACTGGACCCC | |
| | ATCCTTCGACCCTAGGAGCGGTCTT | |
| | TCTGAGGAAGATGCTCAAGTTTTCA | |
| | GAGACATGGCAAGAGATCTGCCCCC | |
| | TCAGTTCACCTCTGGATCACCCTTC | |
| | ACATCAGCATTGGCGGAGGGGTTCA | |
| | CTCCTGAAGATACTCATGACCTGAT | |
| | GGAGGCCTTGACCAGTGTGCTGATA | |
| | CAGATCTGGATCCTGGTGGCTAAGG | |
| | CCATGACCAACATTGACGGCTCTGG | |
| | GGAGGCCAATGAAAGACGTCTTGCA | |
| | AAGTACATCCAAAAAGGACAGCTTA | |
| | ATCGTCAGTTTGCAATTGGTAATCC | |
| | TGCCCGTCTGATAATCCAACAGACA | |
| | ATCAAAAGCTCCTTAACTGTCCGTA | |
| | GGTTCTTGGTCTCTGAGCTTCGTGC | |
| | GTCACGAGGTGCAGTAAAAGAAGGA | |
| | TCCCCTTACTATGCAGCTGTTGGGG | |
| | ATATCCACGCTTACATCTTTAATGC | |
| | GGGATTGACACCATTCTTGACCACC | |
| | TTAAGATACGGGATAGGCACCAAGT | |
| | ACGCCGCTGTTGCACTCAGTGTGTT | |
| | CGCTGCAGATATTGCAAAGTTGAAG | |
| | AGCCTACTTACCCTGTACCAGGACA | |
| | AGGGTGTAGAAGCTGGATACATGGC | |
| | ACTCCTTGAGGATCCAGACTCCATG | |
| | CACTTTGCACCTGGAAACTTCCCAC | |
| | ACATGTACTCCTATGCAATGGGGGT | |
| | AGCTTCTTACCATGATCCTAGCATG | |
| | CGCCAATACCAATACGCCAGGAGGT | |
| | TCCTCAGCCGTCCTTTCTACTTACT | |
| | AGGAAGGGACATGGCCGCCAAGAAC | |
| | ACAGGCACGCTGGATGAGCAACTGG | |
| | CGAAGGAACTGCAAGTATCAGAGAG | |
| | AGATCGCGCCGCATTATCCGCTGCG | |
| | ATTCAATCAGCGATGGAGGGGGGAG | |
| | AGTCCGACGACTTCCCACTGTCGGG | |
| | ATCCATGCCGGCTCTCTCTGAGAAT | |
| | GCGCAACCAGTTACCCCCAGACCTC | |
| | AACAGTCCCAGCTCTCTCCCCCCCA | |
| | ATCATCAAACATGCCCCAATCAGCA | |
| | CCCAGGACCCCAGACTATCAACCCG | |
| | ACTTTGAACTGTAGGCTTCATCACC | |
| | GCACCAACAACAGCCCAAGAAGACC | |
| | ACCCCTCCCCCCACACATCTCACCC | |
| | AGCCACCCATAAAGACTCAGTCCCA | |
| | CGCCCCAGCATCTCCTTCATTTAAT | |
| | TAAAAACCGACCAACAGGGTGGGGA | |
| | AGGAGAGTCATTGGCTACTGCCAAT | |
| | TGTGTGCAGCAATGGATTTTACTGA | |
| | CATTGATGCTGTCAACTCATTGATC | |
| | GAATCATCATCGGCAATCATAGACT | |
| | CCATACAGCATGGAGGGCTGCAACC | |
| | AGCGGGCACCGTCGGCCTATCGCAG | |
| | ATCCCAAAAGGGATAACCAGCGCAT | |
| | TAACCAAGGCCTGGGAGGCTGAGGC | |
| | GGCAACTGCCGGTAATGGGGACACC | |
| | CAACACAAATCTGACAGTCCGGAGG | |
| | ATCATCAGGCCAACGACACAGATTC | |
| | CCCTGAAGACACAGGTACTGACCAG | |
| | ACCACCCAGGAGGCCAACATCGTTG | |
| | AGACACCCCACCCCGAGGTGCTGTC | |
| | AGCAGCCAAAGCCAGACTCAAGAGG | |
| | CCCAAAGCAGGGAGGGACACCCGCG | |
| | ACAACTCCCCTGCGCAACCCGATCA | |
| | TCTTTTAAAGGGGGGCCTCCTGAGC | |
| | CCACAACCAGCAGCATCATGGGTGC | |
| | AAAATCCACCCAGTCATGGAGGTCC | |
| | CGGCACCGCCGATCCCCGCCCATCA | |
| | CAAACTCAGGATCATTCCCCCACCG | |
| | GAGAGAAATGGCGATTGTCACCGAC | |
| | AAAGCAACCGGAGACATTGAACTGG | |
| | TGGAGTGGTGCAACCCGGGGTGCAC | |
| | AGCAGTCCGAATTGAACCCACCAGA | |
| | CTCGACTGTGTATGCGGACACTGCC | |
| | CCACCATCTGTAGCCTCTGCATGTA | |
| | TGACGACTGATCAGGTACAACTACT | |
| | AATGAAGGAGGTTGCTGACATAAAA | |
| | TCACTCCTTCAGGCGTTAGTGAGGA | |
| | ACCTCGCTGTCTTGCCCCAATTGAG | |
| | GAATGAGGTTGCAGCAATCAGAACA | |
| | TCACAGGCCATGATAGAGGGGACAC | |
| | TCAATTCGATCAAGATTCTTGACCC | |
| | TGGGAATTATCAGGAATCATCACTA | |
| | AACAGTTGGTTCAAACCTCGCCAAG | |
| | ATCACACTGTTGTTGTGTCTGGACC | |
| | AGGGAATCCATTGGCCATGCCAACC | |
| | CCAGTCCAAGACAACACCATATTCC | |
| | TGGACGAGCTAGCCAGACCTCATCC | |
| | TAGTGTGGTCAATCCTTCCCCACCC | |
| | ATCACCAACACCAATGTTGACCTTG | |
| | GCCCACAGAAGCAGGCTGCAATAGC | |
| | CTATATCTCCGCTAAATGCAAGGAT | |
| | CCGGGGAAACGAGATCAGCTATCAA | |
| | GGCTCATTGAGCGAGCAACCACCCC | |
| | AAGTGAGATCAACAAAGTTAAAAGA | |
| | CAAGCCCTTGGGCTCTAGATCACTC | |
| | GATCACCCCTCATGGTGATCACAAC | |
| | AATAATCAGAACCCTTCCGAACCAC | |
| | ATGACCAACCCAGCCCACCGCCCAC | |
| | ACCGTCCATCacgcgtGTAGCTGAT | |
| | TTATTCAAAACCGCCACCATGTGCC | |
| | ATCAGCAGCTGGTCATCTCATGGTT | |
| | CTCCCTGGTGTTTCTGGCCTCACCT | |
| | CTGGTCGCAATCTGGGAACTGAAAA | |
| | AGGATGTGTACGTGGTGGAGCTGGA | |
| | CTGGTATCCCGATGCCCCTGGCGAG | |
| | ATGGTGGTGCTGACCTGCGACACAC | |
| | CCGAGGAGGATGGCATCACCTGGAC | |
| | ACTGGATCAGAGCTCCGAGGTGCTG | |
| | GGAAGCGGCAAGACCCTGACAATCC | |
| | AGGTGAAGGAGTTCGGCGACGCCGG | |
| | CCAGTACACCTGTCACAAGGGAGGA | |
| | GAGGTGCTGAGCCACTCCCTGCTGC | |
| | TGCTGCACAAGAAGGAGGATGGCAT | |
| | CTGGTCCACAGACATCCTGAAGGAT | |
| | CAGAAGGAGCCAAAGAACAAGACCT | |
| | TCCTGCGGTGCGAGGCCAAGAATTA | |
| | TAGCGGCCGGTTCACCTGTTGGTGG | |
| | CTGACCACAATCTCCACCGATCTGA | |
| | CATTTTCTGTGAAGTCTAGCAGGGG | |
| | ATCCTCTGACCCACAGGGAGTGACA | |
| | TGCGGAGCAGCCACCCTGAGCGCCG | |
| | AGAGGGTGCGCGGCGATAACAAGGA | |
| | GTACGAGTATTCCGTGGAGTGCCAG | |
| | GAGGACTCTGCCTGTCCAGCAGCAG | |
| | AGGAGTCCCTGCCTATCGAAGTGAT | |
| | GGTGGATGCCGTGCACAAGCTGAAG | |
| | TACGAGAATTATACCAGCTCCTTCT | |
| | TTATCCGGGACATCATCAAGCCCGA | |
| | TCCCCCTAAGAACCTGCAGCTGAAG | |
| | CCTCTGAAGAATAGCAGACAGGTGG | |
| | AGGTGTCCTGGGAGTACCCTGACAC | |
| | CTGGAGCACACCACACTCCTATTTC | |
| | TCTCTGACCTTTTGCGTGCAGGTGC | |
| | AGGGCAAGTCCAAGCGGGAGAAGAA | |
| | GGACAGAGTGTTCACCGATAAGACA | |
| | TCTGCCACCGTGATCTGTAGAAAGA | |
| | ACGCCTCTATCAGCGTGAGGGCCCA | |
| | GGACCGCTACTATTCTAGCTCCTGG | |
| | TCCGAGTGGGCCTCTGTGCCTTGCA | |
| | GCGGCGGAGGAGGAGGAGGATCTAG | |
| | GAATCTGCCAGTGGCAACCCCTGAC | |
| | CCAGGCATGTTCCCCTGCCTGCACC | |
| | ACAGCCAGAACCTGCTGAGGGCCGT | |
| | GTCCAATATGCTGCAGAAGGCCCGC | |
| | CAGACACTGGAGTTTTACCCTTGTA | |
| | CCAGCGAGGAGATCGACCACGAGGA | |
| | CATCACAAAGGATAAGACCTCCACA | |
| | GTGGAGGCCTGCCTGCCACTGGAGC | |
| | TGACCAAGAACGAGTCCTGTCTGAA | |
| | CAGCCGGGAGACAAGCTTCATCACC | |
| | AACGGCTCCTGCCTGGCCTCTAGAA | |
| | AGACAAGCTTTATGATGGCCCTGTG | |
| | CCTGTCTAGCATCTACGAGGACCTG | |
| | AAGATGTATCAGGTGGAGTTCAAGA | |
| | CCATGAACGCCAAGCTGCTGATGGA | |
| | CCCCAAGAGGCAGATCTTTCTGGAT | |
| | CAGAATATGCTGGCCGTGATCGACG | |
| | AGCTGATGCAGGCCCTGAACTTCAA | |
| | TAGCGAGACAGTGCCTCAGAAGTCC | |
| | TCTCTGGAGGAGCCAGATTTCTACA | |
| | AGACCAAGATCAAGCTGTGCATCCT | |
| | GCTGCACGCCTTTCGGATCAGAGCC | |
| | GTGACAATCGACCGCGTGATGTCCT | |
| | ATCTGAATGCTTCCTAATGACCCac | |
| | gcgtCATCCCTTGCCAAACATCCTG | |
| | CCGTAGCTGATTTATTCAAAAGAGC | |
| | TCATTTGATATGACCTGGTAATCAT | |
| | AAAATAGGGTGGGGAAGGTGCTTTG | |
| | CCTGTAAGGGGGCTCCCTCATCTTC | |
| | AGACACGTGCCCGCCATCTCACCAA | |
| | CAGTGCAATGGCAGACATGGACACG | |
| | GTGTATATCAATCTGATGGCAGATG | |
| | ACCCAACCCACCAAAAAGAACTGCT | |
| | GTCCTTTCCTCTCATCCCTGTGACC | |
| | GGTCCTGACGGGAAGAAGGAACTCC | |
| | AACACCAGATCCGGACCCAATCCTT | |
| | GCTCGCCTCAGACAAACAAACTGAA | |
| | CGGTTCATCTTCCTCAACACTTACG | |
| | GATTCATCTATGACACCACACCGGA | |
| | CAAGACAACTTTTTCCACCCCAGAG | |
| | CATATTAATCAGCCTAAGAGGACGA | |
| | CGGTGAGTGCCGCGATGATGACCAT | |
| | TGGCCTGGTTCCCGCCAATATACCC | |
| | CTGAACGAACTAACGGCTACTGTGT | |
| | TCAGCCTTAAAGTAAGAGTGAGGAA | |
| | AAGTGCGAGGTATCGGGAAGTGGTC | |
| | TGGTATCAATGCAATCCAGTACCGG | |
| | CCCTGCTTGCAGCCACCAGGTTTGG | |
| | TCGCCAAGGAGGTCTCGAGTCGAGC | |
| | ACTGGAGTCAGTGTAAAGGCTCCCG | |
| | AGAAGATAGATTGTGAGAAGGATTA | |
| | TACCTACTACCCTTATTTCTTATCT | |
| | GTGTGCTACATCGCCACCTCCAACC | |
| | TGTTCAAGGTACCGAGGATGGTTGC | |
| | TAATGCAACCAACAGTCAATTATAC | |
| | CACCTTACCATGCAGGTCACATTTG | |
| | CCTTTCCAAAAAACATCCCTCCAGC | |
| | CAACCAGAAACTCCTGACACAGGTG | |
| | GATGAGGGATTCGAGGGCACTGTGG | |
| | ATTGCCATTTTGGGAACATGCTGAA | |
| | AAAGGATCGGAAAGGGAACATGAGG | |
| | ACACTGTCCCAGGCGGCAGATAAGG | |
| | TCAGACGAATGAATATTCTTGTTGG | |
| | TATCTTTGACTTGCATGGGCCAACG | |
| | CTCTTCCTGGAGTATACCGGGAAAC | |
| | TGACAAAGGCTCTGCTAGGGTTCAT | |
| | GTCCACCAGCCGAACAGCAATCATC | |
| | CCCATATCTCAGCTCAATCCCATGC | |
| | TGAGTCAACTCATGTGGAGCAGTGA | |
| | TGCCCAGATAGTAAAGTTAAGGGTT | |
| | GTCATAACTACATCCAAACGCGGCC | |
| | CGTGCGGGGGTGAGCAGGAGTATGT | |
| | GCTGGATCCCAAATTCACAGTTAAG | |
| | AAAGAAAAGGCTCGACTCAACCCTT | |
| | TCGAGAAGGCAGCCTAATGATTTAA | |
| | TCCGCAAGATCCCAGAAATCAGACC | |
| | ACTCTATACTATCCACTGATCACTG | |
| | GAAATGTAATTGTACAGTTGATGAA | |
| | TCTGTGAAGAATCAATTAAAAAACC | |
| | GGATCCTTATTAGGGTGGGGAAGTA | |
| | GTTGATTGGGTGTCTAAACAAAAGC | |
| | ATTTCTTCACACCTCCCCGCCACGA | |
| | AACAACCACAATGAGGCTATCAAAC | |
| | ACAATCTTGACCTTGATTCTCATCA | |
| | TACTTACCGGCTATTTGATAGGTGT | |
| | CCACTCCACCGATGTGAATGAGAAA | |
| | CCAAAGTCCGAAGGGATTAGGGGTG | |
| | ATCTTACACCAGGTGCGGGTATTTT | |
| | CGTAACTCAAGTCCGACAGCTCCAG | |
| | ATCTACCAACAGTCTGGGTACCATG | |
| | ATCTTGTCATCAGATTGTTACCTCT | |
| | TCTACCAACAGAGCTTAATGATTGT | |
| | CAAAGGGAAGTTGTCACAGAGTACA | |
| | ATAACACTGTATCACAGCTGTTGCA | |
| | GCCTATCAAAACCAACCTGGATACT | |
| | TTGTTGGCAGATGGTAGCACAAGGG | |
| | ATGTTGATATACAGCCGCGATTCAT | |
| | TGGGGCAATAATAGCCACAGGTGCC | |
| | CTGGCTGTAGCAACGGTAGCTGAGG | |
| | TAACTGCAGCTCAAGCACTATCTCA | |
| | GTCAAAAACGAATGCTCAAAATATT | |
| | CTCAAGTTGAGAGATAGTATTCAGG | |
| | CCACCAACCAAGCAGTTTTTGAAAT | |
| | TTCACAGGGACTCGAAGCAACTGCA | |
| | ACCGTGCTATCAAAACTGCAAACTG | |
| | AGCTCAATGAGAATATCATCCCAAG | |
| | TCTGAACAACTTGTCCTGTGCTGCC | |
| | ATGGGGAATCGCCTTGGTGTATCAC | |
| | TCTCACTCTATTTGACCTTAATGAC | |
| | CACTCTATTTGGGGACCAGATCACA | |
| | AACCCAGTGCTGACGCCAATCTCTT | |
| | ACAGCACCCTATCGGCAATGGCGGG | |
| | TGGTCACATTGGTCCAGTGATGAGT | |
| | AAGATATTAGCCGGATCTGTCACAA | |
| | GTCAGTTGGGGGCAGAACAACTGAT | |
| | TGCTAGTGGCTTAATACAGTCACAG | |
| | GTAGTAGGTTATGATTCCCAGTATC | |
| | AGCTGTTGGTTATCAGGGTCAACCT | |
| | TGTACGGATTCAGGAAGTCCAGAAT | |
| | ACTAGGGTTGTATCACTAAGAACAC | |
| | TAGCAGTCAATAGGGATGGTGGACT | |
| | TTACAGAGCCCAGGTGCCACCCGAG | |
| | GTAGTTGAGCGATCTGGCATTGCAG | |
| | AGCGGTTTTATGCAGATGATTGTGT | |
| | TCTAACTACAACTGATTACATCTGC | |
| | TCATCGATCCGATCTTCTCGGCTTA | |
| | ATCCAGAGTTAGTCAAGTGTCTCAG | |
| | TGGGGCACTTGATTCATGCACATTT | |
| | GAGAGGGAAAGTGCATTACTGTCAA | |
| | CTCCCTTCTTTGTATACAACAAGGC | |
| | AGTCGTCGCAAATTGTAAAGCAGCG | |
| | ACATGTAGATGTAATAAACCGCCAT | |
| | CTATCATTGCCCAATACTCTGCATC | |
| | AGCTCTAGTAACCATCACCACCGAC | |
| | ACTTGTGCTGACCTTGAAATTGAGG | |
| | GTTATCGTTTCAACATACAGACTGA | |
| | ATCCAACTCATGGGTTGCACCAAAC | |
| | TTCACGGTCTCAACCTCACAAATAG | |
| | TATCGGTTGATCCAATAGACATATC | |
| | CTCTGACATTGCCAAAATTAACAAT | |
| | TCTATCGAGGCTGCGCGAGAGCAGC | |
| | TGGAACTGAGCAACCAGATCCTTTC | |
| | CCGAATCAACCCACGGATTGTGAAC | |
| | GACGAATCACTAATAGCTATTATCG | |
| | TGACAATTGTTGTGCTTAGTCTCCT | |
| | TGTAATTGGTCTTATTATTGTTCTC | |
| | GGTGTGATGTACAAGAATCTTAAGA | |
| | AAGTCCAACGAGCTCAAGCTGCTAT | |
| | GATGATGCAGCAAATGAGCTCATCA | |
| | CAGCCTGTGACCACCAAATTGGGGA | |
| | CACCCTTCTAGGTGAATAATCATAT | |
| | CAATCCATTCAATAATGAGCGGGAC | |
| | ATACCAATCACCAACGACTGTGTCA | |
| | CAAGGCCGGTTAGGAATGCACCGGA | |
| | TCTCTCTCCTTCCTTTTTAATTAAA | |
| | AACGGTTGAACTGAGGGTGAGGGGG | |
| | GGGGTGTGCATGGTAGGGTGGGGAA | |
| | GGTAGCCAATTCCTGCCCATTGGGC | |
| | CGACCGTACCAAGAGAAGTCAACAG | |
| | AAGTATAGATGCAGGGCGACATGGA | |
| | GGGTAGCCGTGATAACCTCACAGTA | |
| | GATGATGAATTAAAGACAACATGGA | |
| | GGTTAGCTTATAGAGTTGTATCCCT | |
| | CCTATTGATGGTGAGTGCCTTGATA | |
| | ATCTCTATAGTAATCCTGACGAGAG | |
| | ATAACAGCCAAAGCATAATCACGGC | |
| | GATCAACCAGTCGTATGACGCAGAC | |
| | TCAAAGTGGCAAACAGGGATAGAAG | |
| | GGAAAATCACCTCAATCATGACTGA | |
| | TACGCTCGATACCAGGAATGCAGCT | |
| | CTTCTCCACATTCCACTCCAGCTCA | |
| | ATACACTTGAGGCAAACCTGTTGTC | |
| | CGCCCTCGGAGGTTACACGGGAATT | |
| | GGCCCCGGAGATCTAGAGCACTGTC | |
| | GTTATCCGGTTCATGACTCCGCTTA | |
| | CCTGCATGGAGTCAATCGATTACTC | |
| | ATCAATCAAACAGCTGACTACACAG | |
| | CAGAAGGCCCCCTGGATCATGTGAA | |
| | CTTCATTCCGGCACCAGTTACGACT | |
| | ACTGGATGCACAAGGATCCCATCCT | |
| | TTTCTGTATCATCATCCATTTGGTG | |
| | CTATACACACAATGTGATTGAAACA | |
| | GGTTGCAATGACCACTCAGGTAGTA | |
| | ATCAATATATCAGTATGGGGGTGAT | |
| | TAAGAGGGCTGGCAACGGCTTACCT | |
| | TACTTCTCAACAGTCGTGAGTAAGT | |
| | ATCTGACCGATGGGTTGAATAGAAA | |
| | AAGCTGTTCCGTAGCTGCGGGATCC | |
| | GGGCATTGTTACCTCCTTTGTAGCC | |
| | TAGTGTCAGAGCCCGAACCTGATGA | |
| | CTATGTGTCACCAGATCCCACACCG | |
| | ATGAGGTTAGGGGTGCTAACAAGGG | |
| | ATGGGTCTTACACTGAACAGGTGGT | |
| | ACCCGAAAGAATATTTAAGAACATA | |
| | TGGAGCGCAAACTACCCTGGGGTAG | |
| | GGTCAGGTGCTATAGCAGGAAATAA | |
| | GGTGTTATTCCCATTTTACGGCGGA | |
| | GTGAAGAATGGATCAACCCCTGAGG | |
| | TGATGAATAGGGGAAGATATTACTA | |
| | CATCCAGGATCCAAATGACTATTGC | |
| | CCTGACCCGCTGCAAGATCAGATCT | |
| | TAAGGGCAGAACAATCGTATTATCC | |
| | TACTCGATTTGGTAGGAGGATGGTA | |
| | ATGCAGGGAGTCCTAACATGTCCAG | |
| | TATCCAACAATTCAACAATAGCCAG | |
| | CCAATGCCAATCTTACTATTTCAAC | |
| | AACTCATTAGGATTCATCGGGGCGG | |
| | AATCTAGGATCTATTACCTCAATGG | |
| | TAACATTTACCTTTATCAAAGAAGC | |
| | TCGAGCTGGTGGCCTCACCCCCAAA | |
| | TTTACCTACTTGATTCCAGGATTGC | |
| | AAGTCCGGGTACGCAGAACATTGAC | |
| | TCAGGCGTTAACCTCAAGATGTTAA | |
| | ATGTTACTGTCATTACACGACCATC | |
| | ATCTGGCTTTTGTAATAGTCAGTCA | |
| | AGATGCCCTAATGACTGCTTATTCG | |
| | GGGTTTATTCAGATGTCTGGCCTCT | |
| | TAGCCTTACCTCAGACAGCATATTT | |
| | GCATTTACAATGTACTTACAAGGGA | |
| | AGACGACACGTATTGACCCAGCTTG | |
| | GGCGCTATTCTCCAATCATGTAATT | |
| | GGGCATGAGGCTCGTTTGTTCAACA | |
| | AGGAGGTTAGTGCTGCTTATTCTAC | |
| | CACCACTTGTTTTTCGGACACCATC | |
| | CAAAACCAGGTGTATTGTCTGAGTA | |
| | TACTTGAAGTCAGAAGTGAGCTCTT | |
| | GGGGGCATTCAAGATAGTGCCATTC | |
| | CTCTATCGTGTCTTATAGGCACCTG | |
| | CTTGGTCAAGAACCCTGAGCAGCCA | |
| | TAAAATTAACACTTGATCTTCCTTA | |
| | AAAACACCTATCTAAATTACTGTCT | |
| | GAGATCCCTGATTAGTTACCCTTTC | |
| | AATCAATCAATTAATTTTTAATTAA | |
| | AAACGGAAAAATGGGCCTAGTTCCA | |
| | AGGAAAGGATGGGACCCATTAGGGT | |
| | GGGGAAGGATTACTTTGTTCCTTGA | |
| | CTCGCACCCACGTACACCCAATCCC | |
| | ATTCCTGTCAAGAAGGAACCCTTCC | |
| | CAAACTCACCTTGCAATGTCCAATC | |
| | AGGCAGCTGAGATTATACTACCCAC | |
| | CTTCCATCTTTTATCACCCTTGATC | |
| | GAGAATAAGTGCTTCTACTACATGC | |
| | AATTACTTGGTCTCGTGTTACCACA | |
| | TGATCACTGGAGATGGAGGGCATTC | |
| | GTCAATTTTACAGTGGATCAAGCAC | |
| | ACCTTAAAAATCGTAATCCCCGCTT | |
| | AATGGCCCACATCGATCACACTAAG | |
| | GATAGACTAAGGGCTCATGGTGTCT | |
| | TGGGTTTCCACCAGACTCAGACAAG | |
| | TGAGAGCCGTTTCCGTGTCTTGCTC | |
| | CATCCTGAAACTTTACCTTGGCTAT | |
| | CAGCAATGGGAGGATGCATCAACCA | |
| | GGTTCCCAAGGCATGGCGGAACACT | |
| | CTGAAATCTATCGAGCACAGTGTGA | |
| | AGCAGGAGGCGACTCAACTGAAGTT | |
| | ACTCATGGAAAAAACCTCACTAAAG | |
| | CTAACAGGAGTATCTTACTTATTCT | |
| | CCAATTGCAATCCCGGGAAAACTGC | |
| | AGCGGGAACTATGCCCGTACTAAGT | |
| | GAGATGGCATCAGAACTCTTGTCAA | |
| | ATCCCATCTCCCAATTCCAATCAAC | |
| | ATGGGGGTGTGCTGCTTCAGGGTGG | |
| | CACCATGTAGTCAGCATCATGAGGC | |
| | TCCAACAGTATCAAAGAAGGACAGG | |
| | TAAGGAAGAGAAAGCAATCACTGAA | |
| | GTTCAGTATGGCTCGGACACCTGTC | |
| | TCATTAATGCAGACTACACCGTCGT | |
| | TTTTTCCGCACAGGACCGTGTCATA | |
| | GCAGTCTTGCCTTTCGATGTTGTCC | |
| | TCATGATGCAAGACCTGCTTGAATC | |
| | CCGACGGAATGTCTTGTTCTGTGCC | |
| | CGCTTTATGTATCCCAGAAGCCAAC | |
| | TACATGAGAGGATAAGTACAATACT | |
| | GGCCCTTGGAGACCAACTCGGGAGA | |
| | AAAGCACCCCAAGTCCTGTATGATT | |
| | TCGTAGCTACCCTCGAATCATTTGC | |
| | ATACGCTGCTGTCCAACTTCATGAC | |
| | AACAACCCTATCTACGGTGGGGCTT | |
| | TCTTTGAGTTCAATATCCAAGAACT | |
| | GGAAGCTATTTTGTCCCCTGCACTT | |
| | AATAAGGATCAAGTCAACTTCTACA | |
| | TAAGTCAAGTTGTCTCAGCATACAG | |
| | TAACCTTCCCCCATCTGAATCAGCA | |
| | GAATTGCTATGCTTACTACGCCTGT | |
| | GGGGTCATCCCTTGCTAAACAGTCT | |
| | TGATGCAGCAAAGAAAGTCAGAGAA | |
| | TCTATGTGTGCTGGGAAGGTTCTTG | |
| | ATTATAATGCTATTCGACTAGTTTT | |
| | GTCTTTTTATCATACGTTATTAATC | |
| | AATGGGTATCGGAAGAAACATAAGG | |
| | GTCGCTGGCCAAATGTGAATCAACA | |
| | TTCACTACTCAACCCGATAGTGAAG | |
| | CAGCTTTACTTTGATCAGGAGGAGA | |
| | TCCCACACTCTGTTGCCCTTGAGCA | |
| | CTATTTAGATATCTCGATGATAGAA | |
| | TTTGAGAAGACTTTTGAAGTGGAAC | |
| | TATCTGATAGTCTAAGCATCTTTCT | |
| | GAAGGATAAGTCGATAGCTTTGGAT | |
| | AAACAAGAATGGCACAGTGGTTTTG | |
| | TCTCAGAAGTGACTCCAAAGCACCT | |
| | ACGAATGTCTCGTCATGATCGCAAG | |
| | TCTACCAATAGGCTATTGTTAGCCT | |
| | TTATTAACTCCCCTGAATTCGATGT | |
| | TAAGGAAGAGCTTAAATATTTGACT | |
| | ACAGGTGAGTATGCCACTGACCCAA | |
| | ATTTCAATGTCTCTTACTCACTGAA | |
| | AGAGAAGGAAGTTAAGAAAGAAGGG | |
| | CGCATTTTCGCAAAGATGTCACAGA | |
| | AAATGAGAGCATGCCAGGTTATTTG | |
| | TGAAGAGTTACTAGCACATCATGTG | |
| | GCTCCTTTGTTTAAAGAGAATGGTG | |
| | TTACACAATCGGAGCTATCCCTGAC | |
| | AAAGAATTTGTTGGCTATTAGCCAA | |
| | CTGAGTTACAACTCGATGGCCGCTA | |
| | AGGTGCGATTGCTGAGGCCAGGGGA | |
| | CAAGTTCACCGCTGCACACTATATG | |
| | ACCACAGACCTAAAAAAGTACTGCC | |
| | TTAACTGGCGGCACCAGTCAGTCAA | |
| | ATTGTTCGCCAGAAGCCTGGATCGA | |
| | CTATTTGGGTTAGACCATGCTTTTT | |
| | CTTGGATACACGTCCGTCTCACCAA | |
| | TAGCACTATGTACGTTGCTGACCCA | |
| | TTCAATCCACCAGACTCAGATGCAT | |
| | GCACAAATTTAGACGACAATAAGAA | |
| | CACTGGGATTTTTATTATAAGTGCT | |
| | CGAGGTGGTATAGAAGGCCTTCAAC | |
| | AGAAACTATGGACTGGCATATCAAT | |
| | TGCAATCGCCCAGGCGGCAGCAGCC | |
| | CTCGAGGGCTTACGAATTGCTGCCA | |
| | CTTTGCAGGGGGATAACCAGGTTTT | |
| | AGCGATTACGAAAGAATTCATGACC | |
| | CCAGTCTCGGAGGATGTAATCCACG | |
| | AGCAGCTATCTGAAGCGATGTCGCG | |
| | ATACAAGAGGACTTTCACATACCTT | |
| | AATTATTTAATGGGGCACCAATTGA | |
| | AGGATAAAGAAACCATCCAATCCAG | |
| | TGACTTCTTCGTTTACTCCAAAAGG | |
| | ATCTTCTTCAATGGGTCAATCCTAA | |
| | GTCAATGCCTCAAGAACTTCAGTAA | |
| | ACTCACTACCAATGCCACTACCCTT | |
| | GCTGAGAACACTGTAGCCGGCTGCA | |
| | GTGACATCTCCTCATGCATAGCCCG | |
| | TTGTGTGGAAAACGGGTTGCCTAAG | |
| | GATGCTGCATATGTTCAGAATATAA | |
| | TCATGACTCGGCTTCAACTGTTGCT | |
| | AGATCACTACTATTCTATGCATGGT | |
| | GGCATAAACTCAGAGTTAGAGCAGC | |
| | CAACTCTAAGTATCCCTGTCCGAAA | |
| | CGCAACCTATTTACCATCTCAATTA | |
| | GGCGGTTACAATCATTTGAATATGA | |
| | CCCGACTATTCTGTCGCAATATCGG | |
| | TGACCCGCTTACTAGTTCTTGGGCA | |
| | GAGTCAAAAAGACTAATGGATGTTG | |
| | GCCTTCTCAGTCGTAAGTTCTTAGA | |
| | GGGGATATTATGGAGACCCCCGGGA | |
| | AGTGGGACATTTTCAACACTCATGC | |
| | TTGATCCGTTCGCACTTAACATTGA | |
| | TTACTTAAGGCCACCAGAGACAATA | |
| | ATCCGAAAACACACCCAAAAAGTCT | |
| | TGTTGCAGGATTGTCCTAATCCTCT | |
| | ATTAGCAGGTGTAGTTGACCCGAAC | |
| | TACAACCAGGAATTAGAATTATTAG | |
| | CTCAGTTCCTGCTTGATCGGGAAAC | |
| | CGTTATTCCCAGGGCTGCCCATGCC | |
| | ATCTTTGAACTGTCTGTCTTGGGAA | |
| | GGAAAAAACATATACAAGGATTGGT | |
| | TGATACTACAAAAACAATTATTCAG | |
| | TGCTCATTAGAAAGACAGCCACTGT | |
| | CCTGGAGGAAAGTTGAGAACATTGT | |
| | AACCTACAATGCGCAGTATTTCCTC | |
| | GGGGCCACCCAGCAGGTTGACACCA | |
| | ATATCTCAGAAAGGCAGTGGGTGAT | |
| | GCCAGGTAATTTCAAGAAGCTTGTA | |
| | TCTCTTGACGATTGCTCAGTCACGT | |
| | TGTCCACTGTGTCACGGCGCATTTC | |
| | TTGGGCCAATCTACTTAACTGGAGG | |
| | GCTATAGATGGTTTGGAAACTCCAG | |
| | ATGTGATAGAGAGTATTGATGGCCG | |
| | CCTTGTGCAATCATCCAATCAATGC | |
| | GGCCTATGTAATCAAGGATTGGGCT | |
| | CCTACTCCTGGTTCTTCTTGCCCTC | |
| | CGGGTGTGTGTTCGACCGTCCACAA | |
| | GATTCTCGAGTGGTTCCAAAGATGC | |
| | CATACGTGGGATCCAAAACGGATGA | |
| | GAGACAGACTGCGTCAGTGCAGGCT | |
| | ATACAGGGATCCACATGTCACCTTA | |
| | GAGCAGCATTGAGACTTGTATCACT | |
| | CTACCTTTGGGCCTATGGAGATTCT | |
| | GACATATCATGGCTAGAAGCCGCGA | |
| | CATTGGCTCAAACACGGTGCAATAT | |
| | TTCTCTTGATGACCTGCGGATCCTG | |
| | AGCCCTCTTCCTTCCTCGGCAAATT | |
| | TACACCACAGATTGAATGACGGGGT | |
| | AACACAAGTGAAATTCATGCCCGCC | |
| | ACATCGAGCCGGGTGTCAAAGTTCG | |
| | TCCAAATTTGCAATGACAACCAGAA | |
| | TCTTATCCGTGATGATGGGAGTGTT | |
| | GATTCCAATATGATTTATCAGCAGG | |
| | TTATGATATTAGGGCTTGGAGAGAT | |
| | TGAATGTTTGTTAGCTGACCCAATC | |
| | GATACAAACCCAGAACAACTGATTC | |
| | TTCACCTACACTCTGATAATTCTTG | |
| | CTGTCTCCGGGAGATGCCAACGACC | |
| | GGTTTTGTACCTGCTTTAGGATTGA | |
| | CCCCATGCTTAACTGTCCCAAAGCA | |
| | CAATCCGTATATTTATGATGATAGC | |
| | CCAATACCCGGTGATTTGGATCAGA | |
| | GGCTCATTCAAACCAAATTCTTTAT | |
| | GGGTTCTGACAATCTAGATAATCTT | |
| | GATATCTACCAGCAGCGAGCTTTAC | |
| | TGAGTCGGTGTGTGGCTTATGACAT | |
| | TATCCAATCAGTATTCGCTTGCGAT | |
| | GCACCAGTATCTCAGAAGAATGATG | |
| | CAATCCTTCACACTGACTACCATGA | |
| | AAATTGGATCTCAGAGTTCCGATGG | |
| | GGTGACCCTCGCATAATCCAAGTAA | |
| | CAGCAGGTTACGAGTTAATTCTGTT | |
| | CCTTGCATACCAGCTTTATTATCTC | |
| | AGAGTGAGGGGTGACCGTGCAATCC | |
| | TGTGTTATATTGATAGGATACTCAA | |
| | CAGGATGGTATCTTCCAATCTAGGC | |
| | AGTCTCATCCAGACGCTCTCTCATC | |
| | CGGAGATTAGGAGGAGATTTTCATT | |
| | GAGTGATCAAGGGTTCCTTGTCGAA | |
| | AGGGAGCTAGAGCCAGGTAAGCCAC | |
| | TGGTAAAACAAGCGGTTATGTTCCT | |
| | AAGGGACTCAGTCCGCTGCGCTTTA | |
| | GCAACTATCAAGGCAGGAATTGAGC | |
| | CTGAGATCTCCCGAGGTGGCTGTAC | |
| | CCAGGATGAGCTGAGCTTTACCCTT | |
| | AAGCACTTACTATGTCGGCGTCTCT | |
| | GTATAATTGCTCTCATGCATTCGGA | |
| | AGCAAAGAACTTGGTCAAAGTTAGA | |
| | AACCTTCCAGTAGAGGAAAAAACCG | |
| | CCTTACTATACCAGATGTTGATCAC | |
| | TGAGGCCAATGCCAGGAGATCAGGG | |
| | TCTGCTAGTATCATCATAAGCTTAG | |
| | TTTCAGCACCCCAGTGGGACATTCA | |
| | TACACCAGCGTTGTATTTTGTATCA | |
| | AAGAAAATGCTGGGGATGCTCAAAA | |
| | GGTCAACCACACCCTTGGATATAAG | |
| | TGACCTTTCTGAGAGCCAGAACCTC | |
| | ACACCAACAGAATTGAATGATGTTC | |
| | CTGGTCACATGGCAGAGGAATTTCC | |
| | CTGTTTGTTTAGCAGTTATAACGCT | |
| | ACATATGAAGACACAATTACTTACA | |
| | ATCCAATGACTGAAAAACTCGCAGT | |
| | GCACTTGGACAATGGTTCCACCCCT | |
| | TCCAGAGCGCTTGGTCGTCACTACA | |
| | TCCTGCGACCCCTTGGGCTTTACTC | |
| | GTCTGCATGGTACCGGTCTGCAGCA | |
| | CTATTAGCGTCAGGGGCCCTCAGTG | |
| | GGTTGCCTGAGGGGTCAAGCCTGTA | |
| | CTTGGGAGAGGGGTATGGGACCACC | |
| | ATGACTCTACTTGAGCCCGTTGTCA | |
| | AGTCCTCAACTGTTTACTACCATAC | |
| | ATTGTTTGACCCAACCCGGAATCCT | |
| | TCACAGCGGAACTACAAACCAGAAC | |
| | CGCGGGTATTCACTGATTCCATTTG | |
| | GTACAAGGATGATTTCACACGACCA | |
| | CCTGGTGGCATTGTAAATCTATGGG | |
| | GTGAAGACGTACGTCAGAGTGATAT | |
| | TACACAGAAAGACACGGTTAATTTC | |
| | ATATTATCTCGGGTCCCGCCAAAAT | |
| | CACTCAAATTGATACACGTTGATAT | |
| | TGAGTTCTCCCCAGACTCTGATGTA | |
| | CGGACGCTACTATCTGGCTATTCCC | |
| | ATTGTGCACTATTGGCCTACTGGCT | |
| | ACTGCAACCTGGAGGGCGATTTGCG | |
| | GTTAGAGTTTTCTTAAGTGACCATA | |
| | TCATAGTCAACTTGGTCACTGCCAT | |
| | TCTGTCCGCTTTTGACTCTAATCTG | |
| | GTGTGCATTGCGTCAGGATTGACAC | |
| | ACAAGGATGATGGGGCAGGTTATAT | |
| | TTGTGCAAAGAAGCTTGCAAATGTT | |
| | GAGGCTTCAAGAATTGAGTATTACT | |
| | TGAGGATGGTCCACGGCTGTGTTGA | |
| | CTCATTAAAAATTCCTCATCAATTA | |
| | GGAATCATTAAATGGGCTGAGGGTG | |
| | AAGTGTCCCGACTTACCAAAAAGGC | |
| | GGATGATGAAATAAACTGGCGGTTA | |
| | GGTGATCCAGTTACCAGATCATTTG | |
| | ATCCGGTTTCTGAGCTAATAATTGC | |
| | GCGAACAGGGGGATCAGTATTAATG | |
| | GAATACGGGACTTTTACTAACCTCA | |
| | GGTGTGCGAACTTGGCAGATACATA | |
| | TAAACTTTTGGCTTCAATTGTAGAG | |
| | ACCACCTTAATGGAAATAAGGGTTG | |
| | AGCAAGATCAGTTGGAAGATGATTC | |
| | GAGGAGACAAATCCAGGTAGTCCCT | |
| | GCTTTTAATACAAGATCCGGGGGAA | |
| | GGATCCGTACATTGATTGAGTGTGC | |
| | TCAGCTGCAGGTCATAGATGTTATC | |
| | TGTGTGAACATAGATCACCTCTTTC | |
| | CCAAACACCGACATGCTCTTGTCAC | |
| | ACAACTTACTTACCAGTCAGTGTGC | |
| | CTTGGGGACTTGATTGAAGGCCCCC | |
| | AAATTAAGACATATCTAAGGGCCAG | |
| | GAAGTGGATCCAACGTAGGGGACTC | |
| | AATGAGACAATTAACCATATCATCA | |
| | CTGGACAAGTGTCGCGGAATAAGGC | |
| | AAGGGATTTTTTCAAGAGGCGCCTG | |
| | AAGTTGGTTGGCTTTTCGCTCTGTG | |
| | GCGGTTGGGGCTACCTCTCACTTTA | |
| | GCTGCTTAGATTGTTGATTATTATG | |
| | AATAATCGGAGTCGAAATCGTAAAT | |
| | AGAAAGACATAAAATTGCAAATAAG | |
| | CAATGATCGTATTAATATTTAATAA | |
| | AAAATATGTCTTTTATTTCGT | |

TABLE 3
HETEROLOGOUS SEQUENCES

| | | SEQ ID |
|---|---|---|
| Description | Sequence | NO. |
| Homo sapiens | AGTTCCCTATCACTCTCTTTAATCACTACTCACAGTAACCTCAACTC | SEQ ID |
| interleukin 2 | CTGCCACAATGTACAGGATGCAACTCCTGTCTTGCATTGCACTAAG | NO: 15 |
| (IL2) | TCTTGCACTTGTCACAAACAGTGCACCTACTTCAAGTTCTACAAAG | |
| Genbank: | AAAACACAGCTACAACTGGAGCATTTACTGCTGGATTTACAGATGA | |
| NG_016779.1 | TTTTGAATGGAATTAATGTAAGTATATTTCCTTTCTTACTAAAATTA | |
| | TTACATTTAGTAATCTAGCTGGAGATCATTTCTTAATAACAATGCAT | |
| | TATACTTTCTTAGAATTACAAGAATCCCAAACTCACCAGGATGCTC | |
| | ACATTTAAGTTTTACATGCCCAAGAAGGTAAGTACAATATTTTATGT | |
| | TCAATTTCTGTTTTAATAAAATTCAAAGTAATATGAAAATTTGCACA | |
| | GATGGGACTAATAGCAGCTCATCTGAGGTAAAGAGTAACTTTAATT | |
| | TGTTTTTTTGAAAACCCAAGTTTGATAATGAAGCCTCTATTAAAACA | |
| | GTTTTACCTATATTTTTAATATATATTTGTGTGTTGGTGGGGGTGGG | |
| | AAGAAAACATAAAAATAATATTCTCACTTTATCGATAAGACAATTC | |
| | TAAACAAAAATGTTCATTTATGGTTTCATTTAAAAATGTAAAACTCT | |
| | AAAATATTTGATTATGTCATTTTAGTATGTAAAATACCAAAATCTAT | |
| | TTCCAAGGAGCCCACTTTTAAAAATCTTTTCTTGTTTTAGGAAAGGT | |
| | TTCTAAGTGAGAGGCAGCATAACACTAATAGCACAGAGTCTGGGGC | |
| | CAGATATCTGAAGTGAAATCTCAGCTCTGCCATGTCCTAGCTTTCAT | |
| | GATCTTTGGCAAATTACCTACTCTGTTTGTGATTCAGTTTCATGTCT | |
| | ACTTAAATGAATAACTGTATATACTTAATATGGCTTTGTGAGAATTA | |
| | GTAAGTAAATGTAAAGCACTCAGAACCGTGTCTGGCATAAGGTAAA | |
| | TACCATACAAGCATTAGCTATTATTAGTAGTATTAAAGATAAAATT | |
| | TTCACTGAGAAATACAAAGTAAAATTTTGGACTTTATCTTTTTACCA | |
| | ATAGAACTTGAGATTTATAATGCTATATGACTTATTTTCCAAGATTA | |
| | AAAGCTTCATTAGGTTGTTTTTGGATTCAGATAGAGCATAAGCATA | |
| | ATCATCCAAGCTCCTAGGCTACATTAGGTGTGTAAAGCTACCTAGT | |
| | AGCTGTGCCAGTTAAGAGAGAATGAACAAAATCTGGTGCCAGAAA | |
| | GAGCTTGTGCCAGGGTGAATCCAAGCCCAGAAAATAATAGGATTTA | |
| | AGGGGACACAGATGCAATCCCATTGACTCAAATTCTATTAATTCAA | |
| | GAGAAATCTGCTTCTAACTACCCTTCTGAAAGATGTAAAGGAGACA | |
| | GCTTACAGATGTTACTCTAGTTTAATCAGAGCCACATAATGCAACT | |
| | CCAGCAACATAAAGATACTAGATGCTGTTTTCTGAAGAAAATTTCT | |
| | CCACATTGTTCATGCCAAAAACTTAAACCCGAATTTGTAGAATTTGT | |
| | AGTGGTGAATTGAAAGCGCAATAGATGGACATATCAGGGGATTGG | |
| | TATTGTCTTGACCTACCTTTCCCACTAAAGAGTGTTAGAAAGATGA | |
| | GATTATGTGCATAATTTAGGGGGTGGTAGAATTCATGGAAATCTAA | |
| | GTTTGAAACCAAAAGTAATGATAAACTCTATTCATTTGTTCATTTAA | |
| | CCCTCATTGCACATTTACAAAAGATTTTAGAAACTAATAAAAATAT | |
| | TTGATTCCAAGGATGCTATGTTAATGCTATAATGAGAAAGAAATGA | |
| | AATCTAATTCTGGCTCTACCTACTTATGTGGTCAAATTCTGAGATTT | |
| | AGTGTGCTTATTTATAAAGTGGAGATGATACTTCACTGCCTACTTCA | |
| | AAAGATGACTGTGAGAAGTAAATGGGCCTATTTTGGAGAAAATTCT | |
| | TTTAAATTGTAATATACCATAGAAATATGAAATATTATATATAATAT | |
| | AGAATCAAGAGGCCTGTCCAAAAGTCCTCCCAAAGTATTATAATTT | |
| | TTTATTTCACTGGGACAAACATTTTTAAAATGCATCTTAATGTAGTG | |
| | ATTGTAGAAAAGTAAAAATTTAAGACATATTTAAAAATGTGTCTTG | |
| | CTCAAGGCTATATTGAGAGCCACTACTACATGATTATTGTTACCTAG | |
| | TGTAAAATGTTGGGATTGTGATAGATGGCATCCAAGAGTTCCTTCT | |
| | CTCTCAACATTCTGTGATTCTTAACTCTTAGACTATCAAATATTATA | |
| | ATCATAGAATGTGATTTTTATGCTTCCACATTCTAACTCATCTGGTT | |
| | CTAATGATTTTCTATGCAGATTGGAAAAGTAATCAGCCTACATCTGT | |
| | AATAGGCATTTAGATGCAGAAAGTCTAACATTTTGCAAAGCCAAAT | |
| | TAAGCTAAAACCAGTGAGTCAACTATCACTTAACGCTAGTCATAGG | |
| | TACTTGAGCCCTAGTTTTTCCAGTTTTATAATGTAAACTCTACTGGT | |
| | CCATCTTTACAGTGACATTGAGAACAGAGAGAATGGTAAAAACTAC | |
| | ATACTGCTACTCCAAATAAAATAAATTGGAAATTAATTTCTGATTCT | |
| | GACCTCTATGTAAACTGAGCTGATGATAATTATTATTCTAGGCCAC | |
| | AGAACTGAAACATCTTCAGTGTCTAGAAGAAGAACTCAAACCTCTG | |
| | GAGGAAGTGCTAAATTTAGCTCAAAGCAAAAACTTTCACTTAAGAC | |
| | CCAGGGACTTAATCAGCAATATCAACGTAATAGTTCTGGAACTAAA | |
| | GGTAAGGCATTACTTTATTTGCTCTCCTGGAAATAAAAAAAAAAAA | |
| | GTAGGGGGAAAAGTACCACATTTTAAAGTGACATAACATTTTTGGT | |
| | ATTTGTAAAGTACCCATGCATGTAATTAGCCTACATTTTAAGTACAC | |
| | TGTGAACATGAATCATTTCTAATGTTAAATGATTAACTGGGGAGTA | |
| | TAAGCTACTGAGTTTGCACCTACCATCTACTAATGGACAAGCCTCA | |
| | TCCCAAACTCCATCACCTTTCATATTAACACAAAACTGGGAGTGAG | |
| | AGAAGGTACTGAGTTGAGTTTCACAGAAAGCAGGCAGATTTTACTA | |
| | TATATTTTTCAATTCCTTCAGATCATTTACTGGAATAGCCAATACTG | |
| | ATTACCTGAAAGGCTTTTCAAATGGTGTTTCCTTATCATTTGATGGA | |
| | AGGACTACCCATAAGAGATTTGTCTTAAAAAAAAAAACTGGAGCC | |
| | ATTAAAATGGCCAGTGGACTAAACAAACAACAATCTTTTTAGAGGC | |
| | AATCCCCACTTTCAGAATCTTAAGTATTTTTAAATGCACAGGAAGC | |
| | ATAAAATATGCAAGGGACTCAGGTGATGTAAAAGAGATTCACTTTT | |
| | GTCTTTTTATATCCCGTCTCCTAAGGTATAAAATTCATGAGTTAATA | |
| | GGTATCCTAAATAAGCAGCATAAGTATAGTAGTAAAAGACATTCCT | |
| | AAAAGTAACTCCAGTTGTGTCCAAATGAATCACTTATTAGTGGACT | |
| | GTTTCAGTTGAATTAAAAAAATACATTGAGATCAATGTCATCTAGA | |
| | CATTGACAGATTCAGTTCCTTATCTATGGCAAGAGTTTTACTCTAAA | |
| | ATAATTAACATCAGAAAACTCATTCTTAACTCTTGATACAAATTTAA | |
| | GACAAAACCATGCAAAAATCTGAAAACTGTGTTTCAAAAGCCAAA | |
| | CACTTTTTAAAATAAAAAAATCCCAAGATATGACAATATTTAAACA | |
| | ATTATGCTTAAGAGGATACAGAACACTGCAACAGTTTTTTAAAAGA | |
| | GAATACTTATTTAAAGGGAACACTCTATCTCACCTGCTTTTGTTCCC | |
| | AGGGTAGGAATCACTTCAAATTTGAAAAGCTCTCTTTTAAATCTCA | |
| | CTATATATCAAAATATTTCCTCCTTAGCTTATCAACTAGAGGAAGCG | |
| | TTTAAATAGCTCCTTTCAGCAGAGAAGCCTAATTTCTAAAAAGCCA | |
| | GTCCACAGAACAAAATTTCTAATGTTTAAACTTTTAAAAGTTGGCA | |
| | AATTCACCTGCATTGATACTATGATGGGGTAGGGATAGGTGTAAGT | |
| | ATTTATGAAGATGTTCTTCACACAAATTTATCCCAAACAGAAGCAT | |
| | GTCCTAGCTTACTCTAGTGTAGTTCTGTTCTGCTTTGGGGAAAATAT | |
| | AAGGAGATTCACTTAAGTAGAAAAATAGGAGACTCTAATCAAGATT | |
| | TAGAAAAGAAGAAAGTATAATGTGCATATCAATTCATACATTTAAC | |
| | TTACACAAATATAGGTGTACATTCAGAGGAAAAGCGATCAAGTTTA | |
| | TTTCACATCCAGCATTTAATATTTGTCTAGATCTATTTTTATTTAAAT | |
| | CTTTATTTGCACCCAATTTAGGGAAAAAATTTTTGTGTTCATTGACT | |
| | GAATTAACAAATGAGGAAAATCTCAGCTTCTGTGTTACTATCATTT | |
| | GGTATCATAACAAAATATGTAATTTTGGCATTCATTTTGATCATTTC | |
| | AAGAAAATGTGAATAATTAATATGTTTGGTAAGCTTGAAAATAAAG | |
| | GCAACAGGCCTATAAGACTTCAATTGGGAATAACTGTATATAAGGT | |
| | AAACTACTCTGTACTTTAAAAAATTAACATTTTTCTTTTATAGGGAT | |
| | CTGAAACAACATTCATGTGTGAATATGCTGATGAGACAGCAACCAT | |
| | TGTAGAATTTCTGAACAGATGGATTACCTTTTGTCAAAGCATCATCT | |
| | CAACACTGACTTGATAATTAAGTGCTTCCCACTTAAAACATATCAG | |
| | GCCTTCTATTTATTTAAATATTTAAATTTTATATTTATTGTTGAATGT | |
| | ATGGTTTGCTACCTATTGTAACTATTATTCTTAATCTTAAAACTATA | |
| | AATATGGATCTTTTATGATTCTTTTTGTAAGCCCTAGGGGCTCTAAA | |
| | ATGGTTTCACTTATTTATCCCAAAATATTTATTATTATGTTGAATGTT | |
| | AAATATAGTATCTATGTAGATTGGTTAGTAAAACTATTTAATAAATT | |
| | TGATAAATATAAA | |
| hIL-12V3 | ATGGGTCACCAGCAGTTGGTCATCTCTTGGTTTTCCCTGGTTT | SEQ ID |
| | TTCTGGCATCTCCCCTCGTGGCCATATGGGAACTGAAGAAAG | NO: 16 |
| | ATGTTTATGTCGTAGAATTGGATTGGTATCCGGATGCCCCTG | |
| | GAGAAATGGTGGTCCTCACCTGTGACACCCCTGAAGAAGAT | |
| | GGTATCACCTGGACCTTGGACCAGAGCAGTGAGGTCTTAGGC | |
| | TCTGGCAAAACCCTGACCATCCAAGTCAAAGAGTTTGGAGAT | |
| | GCTGGCCAGTACACCTGTCACAAAGGAGGCGAGGTTCTAAG | |
| | CCATTCGCTCCTGCTGCTTCACAAAAAGGAAGATGGAATTTG | |
| | GTCCACTGATATTTTAAAGGACCAGAAAGAACCCAAAAATA | |
| | AGACCTTTCTAAGATGCGAGGCCAAGAATTATTCTGGACGTT | |
| | TCACCTGCTGGTGGCTGACGACAATCAGTACTGATTTGACAT | |
| | TCAGTGTCAAAAGCAGCAGAGGCTCTTCTGACCCCCAAGGG | |
| | GTGACGTGCGGAGCTGCTACACTCTCTGCAGAGAGAGTCAG | |
| | AGGGGACAACAAGGAGTATGAGTACTCAGTGGAGTGCCAGG | |
| | AGGACAGTGCCTGCCCAGCTGCTGAGGAGAGTCTGCCCATTG | |
| | AGGTCATGGTGGATGCCGTTCACAAGCTCAAGTATGAAAACT | |
| | ACACCAGCAGCTTCTTCATCAGGGACATCATCAAACCTGACC | |
| | CACCCAAGAACTTGCAGCTGAAGCCATTAAAGAATTCTCGGC | |
| | AGGTGGAGGTCAGCTGGGAGTACCCTGACACCTGGAGTACT | |
| | CCACATTCCTACTTCTCCCTGACATTCTGCGTTCAGGTCCAGG | |
| | GCAAGAGCAAGAGAGAAAAGAAAGATAGAGTCTTCACGGAC | |
| | AAGACCTCAGCCACGGTCATCTGCCGCAAAAATGCCAGCATT | |
| | AGCGTGCGGGCCCAGGACCGCTACTATAGCTCATCTTGGAGC | |
| | GAATGGGCATCTGTGCCCTGCAGTGGTGGCGGTGGCGGCG | |
| | GATCTAGAAACCTCCCCGTGGCCACTCCAGACCCAGGAATG | |
| | TTCCCATGCCTTCACCACTCCCAAAACCTGCTGAGGGCCGTC | |
| | AGCAACATGCTCCAGAAGGCCAGACAAACTCTAGAATTTTAC | |
| | CCTTGCACTTCTGAAGAGATTGATCATGAAGATATCACAAAA | |
| | GATAAAACCAGCACAGTGGAGGCCTGTTTACCATTGGAATTA | |
| | ACCAAGAATGAGAGTTGCCTAAATTCCAGAGAGACCTCTTTC | |
| | ATAACTAATGGGAGTTGCCTGGCCTCCAGAAAGACCTCTTTT | |
| | ATGATGGCCCTGTGCCTTAGTAGTATTTATGAAGACTCGAAG | |
| | ATGTACCAGGTGGAGTTCAAGACCATGAATGCAAAGCTTCTG | |
| | ATGGATCCTAAGAGGCAGATCTTTCTAGATCAAAACATGCTG | |
| | GCAGTTATTGATGAGCTGATGCAGGCCCTGAATTTCAACAGT | |
| | GAGACTGTGCCACAAAAATCCTCCCTTGAAGAACCGGATTTT | |
| | TATAAAACTAAAATCAAGCTCTGCATACTTCTTCATGCTTTCA | |
| | GAATTCGGGCAGTGACTATTGATAGAGTGATGAGCTATCTGA | |
| | ATGCTTCCTAAT | |
| OPT hIL 12 | ATGTGCCATCAGCAGCTGGTCATCTCATGGTTCTCCCTGGTGTTTCT | SEQ ID |
| | GGCCTCACCTCTGGTCGCAATCTGGGAACTGAAAAAGGATGTGTAC | NO: 17 |
| | GTGGTGGAGCTGGACTGGTATCCCGATGCCCCTGGCGAGATGGTGG | |
| | TGCTGACCTGCGACACACCCGAGGAGGATGGCATCACCTGGACACT | |
| | GGATCAGAGCTCCGAGGTGCTGGGAAGCGGCAAGACCCTGACAAT | |
| | CCAGGTGAAGGAGTTCGGCGACGCCGGCCAGTACACCTGTCACAA | |
| | GGGAGGAGAGGTGCTGAGCCACTCCCTGCTGCTGCTGCACAAGAA | |
| | GGAGGATGGCATCTGGTCCACAGACATCCTGAAGGATCAGAAGGA | |
| | GCCAAAGAACAAGACCTTCCTGCGGTGCGAGGCCAAGAATTATAG | |
| | CGGCCGGTTCACCTGTTGGTGGCTGACCACAATCTCCACCGATCTG | |
| | ACATTTTCTGTGAAGTCTAGCAGGGGATCCTCTGACCCACAGGGAG | |
| | TGACATGCGGAGCAGCCACCCTGAGCGCCGAGAGGGTGCGCGGCG | |
| | ATAACAAGGAGTACGAGTATTCCGTGGAGTGCCAGGAGGACTCTGC | |
| | CTGTCCAGCAGCAGAGGAGTCCCTGCCTATCGAAGTGATGGTGGAT | |
| | GCCGTGCACAAGCTGAAGTACGAGAATTATACCAGCTCCTTCTTTA | |
| | TCCGGGACATCATCAAGCCCGATCCCCCTAAGAACCTGCAGCTGAA | |
| | GCCTCTGAAGAATAGCAGACAGGTGGAGGTGTCCTGGGAGTACCCT | |
| | GACACCTGGAGCACACCACACTCCTATTTCTCTCTGACCTTTTGCGT | |
| | GCAGGTGCAGGGCAAGTCCAAGCGGGAGAAGAAGGACAGAGTGTT | |
| | CACCGATAAGACATCTGCCACCGTGATCTGTAGAAAGAACGCCTCT | |
| | ATCAGCGTGAGGGCCCAGGACCGCTACTATTCTAGCTCCTGGTCCG | |
| | AGTGGGCCTCTGTGCCTTGCAGCGGCGGAGGAGGAGGAGGATCTA | |
| | GGAATCTGCCAGTGGCAACCCCTGACCCAGGCATGTTCCCCTGCCT | |
| | GCACCACAGCCAGAACCTGCTGAGGGCCGTGTCCAATATGCTGCAG | |
| | AAGGCCCGCCAGACACTGGAGTTTTACCCTTGTACCAGCGAGGAGA | |
| | TCGACCACGAGGACATCACAAAGGATAAGACCTCCACAGTGGAGG | |
| | CCTGCCTGCCACTGGAGCTGACCAAGAACGAGTCCTGTCTGAACAG | |
| | CCGGGAGACAAGCTTCATCACCAACGGCTCCTGCCTGGCCTCTAGA | |
| | AAGACAAGCTTTATGATGGCCCTGTGCCTGTCTAGCATCTACGAGG | |
| | ACCTGAAGATGTATCAGGTGGAGTTCAAGACCATGAACGCCAAGCT | |
| | GCTGATGGACCCCAAGAGGCAGATCTTTCTGGATCAGAATATGCTG | |
| | GCCGTGATCGACGAGCTGATGCAGGCCCTGAACTTCAATAGCGAGA | |
| | CAGTGCCTCAGAAGTCCTCTCTGGAGGAGCCAGATTTCTACAAGAC | |
| | CAAGATCAAGCTGTGCATCCTGCTGCACGCCTTTCGGATCAGAGCC | |
| | GTGACAATCGACCGCGTGATGTC CTATCTGAATGCTTCCTAATGA | |
| hIL-15Ra-IL15 | ATGGCGCCGCGCCGCGCGCGCGGCTGCCGCACCCTGGG | SEQ ID |
| (signal sequence | CCTGCCGGCGCTGCTGCTGCTGCTGCTGCTGCGCCCGCC | NO: 18 |
| underlined, flag- | GGCGACCCGCGGCGATTATAAAGATGATGATGATAAA | |
| tag in bold, | ATTGAAGGCCGCATTACCTGCCCGCCGCCGATGAGCGT | |
| linker double | GGAACATGCGGATATTTGGGTGAAAAGCTATAGCCTGT | |
| underlined and | ATAGCCGCGAACGCTATATTTGCAACAGCGGCTTTAAA | |
| human IL-15 in | CGCAAAGCGGGCACCAGCAGCCTGACCGAATGCGTGCT | |
| italics) | GAACAAAGCGACCAACGTGGCGCATTGGACCACCCCGA | |
| | GCCTGAAATGCATTCGCGATCCGGCGCTGGTGCATCAG | |
| | 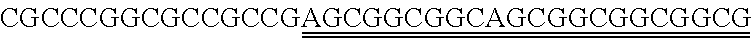 | |
| | 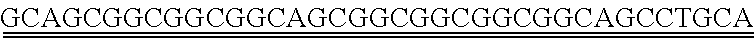 | |
| | GATGCGCATTAGCAAACCGCATCTGCGCAGCATTAGCATTC | |
| | AGTGCTATCTGTGCCTGCTGCTGAACAGCCATTTTCTGACC | |
| | GAAGCGGGCATTCATGTGTTTATTCTGGGCTGCTTTAGCGC | |
| | GGGCCTGCCGAAAACCGAAGCGAACTGGGTGAACGTGATT | |
| | AGCGATCTGAAAAAAATTGAAGATCTGATTCAGAGCATGCAT | |
| | ATTGATGCGACCCTGTATACCGAAAGCGATGTGCATCCGAG | |
| | CTGCAAAGTGACCGCGATGAAATGCTTTCTGCTGGAACTGC | |
| | AGGTGATTAGCCTGGAAAGCGGCGATGCGAGCATTCATGA | |
| | TACCGTGGAAAACCTGATTATTCTGGCGAACAACAGCCTGA | |
| | GCAGCAACGGCAACGTGACCGAAAGCGGCTGCAAAGAATG | |
| | CGAAGAACTGGAAGAAAAAAACATTAAAGAATTTCTGCAGA | |
| | GCTTTGTGCATATTGTGCAGATGTTTATTAACACCAGC | |
| HPV16 E6 | ATGCACCAAAAGAGAACTGCAATGTTTCAGGACCCACAGGAGCGA | SEQ ID |
| | CCCAGAAAGTTACCACAGTTATGCACAGAGCTGCAAACAACTATAC | NO: 19 |
| | ATGATATAATATTAGAATGTGTGTACTGCAAGCAACAGTTACTGCG | |
| | ACGTGAGGTATATGACTTTGCTTTTCGGGATTTATGCATAGTATATA | |
| | GAGATGGGAATCCATATGCTGTATGTGATAAATGTTTAAAGTTTTA | |
| | TTCTAAAATTAGTGAGTATAGACATTATTGTTATAGTTTGTATGGAA | |
| | CAACATTAGAACAGCAATACAACAAACCGTTGTGTGATTTGTTAAT | |
| | TAGGTGTATTAACTGTCAAAAGCCACTGTGTCCTGAAGAAAAGCAA | |
| | AGACATCTGGACAAAAAGCAAAGATTCCATAATATAAGGGGTCGG | |
| | TGGACCGGTCGATGTATGTCTTGTTGCAGATCATCAAGAACACGTA | |
| | GAGAAACCCAGCTGTAA | |
| HPV16 E7 | ATGCATGGAGATACACCTACATTGCATGAATATATGTTAGATTTGC | SEQ ID |
| | AACCAGAGACAACTGATCTCTACTGTTATGAGCAATTAAATGACAG | NO: 20 |
| | CTCAGAGGAGGAGGATGAAATAGATGGTCCAGCTGGACAAGCAGA | |
| | ACCGGACAGAGCCCATTACAATATTGTAACCTTTTGTTGCAAGTGT | |
| | GACTCTACGCTTCGGTTGTGCGTACAAAGCACACACGTAGACATTC | |
| | GTACTTTGGAAGACCTGTTAATGGGCACACTAGGAATTGTGTGCCC | |
| | CATCTGTTCTCAGAAACCATAA | |
| Human gene for | TTCTCAGAGTGGCTGCAGTCTCGCTGCTGGATGTGCACATGGTGGT | SEQ ID |
| granulocyte- | CATTCCCTCTGCTCACAGGGGCAGGGGTCCCCCCTTACTGGACTGA | NO: 21 |
| macrophage | GGTTGCCCCCTGCTCCAGGTCCTGGGTGGGAGCCCATGTGAACTGT | |
| colony | CAGTGGGGCAGGTCTGTGAGAGCTCCCCTCACACTCAAGTCTCTCT | |
| stimulating | CACAGTGGCCAGAGAAGAGGAAGGCTGGAGTCAGAATGAGGCACC | |
| factor (GM-CSF) | AGGGCGGGCATAGCCTGCCCAAAGGCCCCTGGGATTACAGGCAGG | |
| GenBank: | ATGGGGAGCCCTATCTAAGTGTCTCCCACGCCCCACCCCAGCCATT | |
| X03021.1 | CCAGGCCAGGAAGTCCAAACTGTGCCCCTCAGAGGGAGGGGGCAG | |
| | CCTCAGGCCCATTCAGACTGCCCAGGGAGGGCTGGAGAGCCCTCAG | |
| | GAAGGCGGGTGGGTGGGCTGTCGGTTCTTGGAAAGGTTCATTAATG | |
| | AAAACCCCCAAGCCTGACCACCTAGGGAAAAGGCTCACCGTTCCCA | |
| | TGTGTGGCTGATAAGGGCCAGGAGATTCCACAGTTCAGGTAGTTCC | |
| | CCCGCCTCCCTGGCATTTTGTGGTCACCATTAATCATTTCCTCTGTG | |
| | TATTTAAGAGCTCTTTTGCCAGTGAGCCCAGCTACACAGAGAGAAA | |
| | GGCTAAAGTTCTCTGGAGGATGTGGCTGCAGAGCCTGCTGCTCTTG | |
| | GGCACTGTGGCCTGCAGCATCTCTGCACCCGCCCGCTCGCCCAGCC | |
| | CCAGCACGCAGCCCTGGGAGCATGTGAATGCCATCCAGGAGGCCC | |
| | GGCGTCTCCTGAACCTGAGTAGAGACACTGCTGCTGAGATGGTAAG | |
| | TGAGAGAATGTGGGCCTGTGCTAGGCACCAGTGGCCCTGACTGGCC | |
| | ACGCCTGTCAGCTTGATAACATGACATTTTCCTTTTCTACAGAATGA | |
| | AACAGTAGAAGTCATCTCAGAAATGTTTGACCTCCAGGTAAGATGC | |
| | TTCTCTCTGACATAGCTTTCCAGAAGCCCCTGCCCTGGGGTGGAGGT | |
| | GGGGACTCCATTTTAGATGGCACCACACAGGGTTGTCCACTTTCTCT | |
| | CCAGTCAGCTGGCTGCAGGAGGAGGGGGTAGCAACTGGGTGCTCA | |
| | AGAGGCTGCTGGCCGTGCCCCTATGGCAGTCACATGAGCTCCTTTA | |
| | TCAGCTGAGCGGCCATGGGCAGACCTAGCATTCAATGGCCAGGAGT | |
| | CACCAGGGGACAGGTGGTAAAGTGGGGGTCACTTCATGAGACAGG | |
| | AGCTGTGGGTTTGGGGCGCTCACTGTGCCCCGAGACCAAGTCCTGT | |
| | TGAGACAGTGCTGACTACAGAGAGGCACAGAGGGGTTTCAGGAA | |
| | CAACCCTTGCCCACCCAGCAGGTCCAGGTGAGGCCCCACCCCCCTC | |
| | TCCCTGAATGATGGGGTGAGAGTCACCTCCTTCCCTAAGGCTGGGC | |
| | TCCTCTCCAGGTGCCGCTGAGGGTGGCCTGGGCGGGGCAGTGAGAA | |
| | GGGCAGGTTCGTGCCTGCCATGGACAGGGCAGGGTCTATGACTGGA | |
| | CCCAGCCTGTGCCCCTCCCAAGCCCTACTCCTGGGGGCTGGGGGCA | |
| | GCAGCAAAAAGGAGTGGTGGAGAGTTCTTGTACCACTGTGGGCACT | |
| | TGGCCACTGCTCACCGACGAACGACATTTTCCACAGGAGCCGACCT | |
| | GCCTACAGACCCGCCTGGAGCTGTACAAGCAGGGCCTGCGGGGCA | |
| | GCCTCACCAAGCTCAAGGGCCCCTTGACCATGATGGCCAGCCACTA | |
| | CAAGCAGCACTGCCCTCCAACCCCGGTGAGTGCCTACGGCAGGGCC | |
| | TCCAGCAGGAATGTCTTAATCTAGGGGGTGGGGTCGACATGGGGAG | |
| | AGATCTATGGCTGTGGCTGTTCAGGACCCCAGGGGGTTTCTGTGCC | |
| | AACAGTTATGTAATGATTAGCCCTCCAGAGAGGAGGCAGACAGCCC | |
| | ATTTCATCCCAAGGAGTCAGAGCCACAGAGCGCTGAAGCCCACAGT | |
| | GCTCCCCAGCAGGAGCTGCTCCTATCCTGGTCATTATTGTCATTACG | |
| | GTTAATGAGGTCAGAGGTGAGGGCAAACCCAAGGAAACTTGGGGC | |
| | CTGCCCAAGGCCCAGAGGAAGTGCCCAGGCCCAAGTGCCACCTTCT | |
| | GGCAGGACTTTCCTCTGGCCCCACATGGGGTGCTTGAATTGCAGAG | |
| | GATCAAGGAAGGGAGGCTACTTGGAATGGACAAGGACCTCAGGCA | |
| | CTCCTTCCTGCGGGAAGGGAGCAAAGTTTGTGGCCTTGACTCCACT | |
| | CCTTCTGGGTGCCCAGAGACGACCTCAGCCCAGCTGCCCTGCTCTG | |
| | CCCTGGGACCAAAAAGGCAGGCGTTTGACTGCCCAGAAGGCCAAC | |
| | CTCAGGCTGGCACTTAAGTCAGGCCCTTGACTCTGGCTGCCACTGG | |
| | CAGAGCTATGCACTCCTTGGGGAACACGTGGGTGGCAGCAGCGTCA | |
| | CCTGACCCAGGTCAGTGGGTGTGTCCTGGAGTGGGCCTCCTGGCCT | |
| | CTGAGTTCTAAGAGGCAGTAGAGAAACATGCTGGTGCTTCCTTCCC | |
| | CCACGTTACCCACTTGCCTGGACTCAAGTGTTTTTTATTTTTCTTTTT | |
| | TTAAAGGAAACTTCCTGTGCAACCCAGATTATCACCTTTGAAAGTTT | |
| | CAAAGAGAACCTGAAGGACTTTCTGCTTGTCATCCCCTTTGACTGCT | |
| | GGGAGCCAGTCCAGGAGTGAGACCGGCCAGATGAGGCTGGCCAAG | |
| | CCGGGGAGCTGCTCTCTCATGAAACAAGAGCTAGAAACTCAGGATG | |
| | GTCATCTTGGAGGGACCAAGGGGTGGGCCACAGCCATGGTGGGAG | |
| | TGGCCTGGACCTGCCCTGGGCACACTGACCCTGATACAGGCATGGC | |
| | AGAAGAATGGGAATATTTTATACTGACAGAAATCAGTAATATTTAT | |
| | ATATTTATATTTTTAAAATATTTATTTATTTATTTATTTAAGTTCATA | |
| | TTCCATATTTATTCAAGATGTTTTACCGTAATAATTATTATTAAAAA | |
| | TATGCTTCTACTTGTCCAGTGTTCTAGTTTGTTTTTAACCATGAGCA | |
| | AATGCCAT | |
| Human IL-12 | MGHQQLVISWFSLVFLASPLVAIWELKKDVYVVELDWYP | SEQ ID |
| fusion protein | DAPGEMVVLTCDTPEEDGITWTLDQSSEVLGSGKTLTIQV | NO: 34 |
| (Linker | KEFGDAGQYTCHKGGEVLSHSLLLLHKKEDGIWSTDILKD | |
| underlined) | QKEPKNKTFLRCEAKNYSGRFTCWWLTTISTDLTFSVKSS | |
| | RGSSDPQGVTCGAATLSAERVRGDNKEYEYSVECQEDSA | |
| | CPAAEESLPIEVMVDAVHKLKYENYTSSFFIRDIIKPDPPKN | |
| | LQLKPLKNSRQVEVSWEYPDTWSTPHSYFSLTFCVQVQG | |
| | KSKREKKDRVFTDKTSATVICRKNASISVRAQDRYYSSSW | |
| | 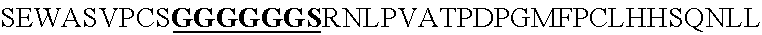 | |
| | RAVSNMLQKARQTLEFYPCTSEEIDHEDITKDKTSTVEAC | |
| | LPLELTKNESCLNSRETSFITNGSCLASRKTSFMMALCLSSI | |
| | YEDSKMYQVEFKTMNAKLLMDPKRQIFLDQNMLAVIDEL | |
| | MQALNFNSETVPQKSSLEEPDFYKTKIKLCILLHAFRIRAV | |
| | TIDRVMSYLNAS | |
| Human IL-15Ra- | MAPRRARGCRTLGLPALLLLLLLRPPATRGDYKDDDDKI | SEQ ID |
| IL15 (signal | EGRITCPPPMSVEHADIWVKSYSLYSRERYICNSGFKRKA | NO: 37 |
| sequence | GTSSLTECVLNKATNVAHWTTPSLKCIRDPALVHQRPAPP | |
| underlined, flag- | 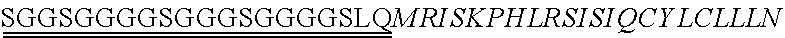 | |
| tag in bold, | SHFLTEAGIHVFILGCFSAGLPKTEANWVNVISDLKKIEDLIQS | |
| linker sequence | MHIDATLYTESDVHPSCKVTAMKCELLELQVISLESGDASIHD | |
| double | TVENLIILANNSLSSNGNVTESGCKECEELEEKNIKEFLQSFVH | |
| underlined, | IVQMFINTS | |
| human IL-15 | | |
| italics) | | |
| Human IL-15Ra- | ITCPPPMSVEHADIWVKSYSLYSRERYICNSGFKRKAGTSS | SEQ ID |
| sushi | LTECVLNKATNVAHWTTPSLKCIRDPALVHQRPAPP | NO: 39 |
| Human IL-15 | MRISKPHLRSISIQCYLCLLLNSHFLTEAGIHVFILGCFSAGL | SEQ ID |
| | PKTEANWVNVISDLKKIEDLIQSMHIDATLYTESDVHPSCK | NO: 40 |
| | VTAMKCFLLELQVISLESGDASIHDTVENLIILANNSLSSNG | |
| | NVTESGCKECEELEEKNIKEFLQSFVHIVQMFINTS | |
| Human IL-12 | MGHQQLVISWFSLVFLASPLVAIWELKKDVYVVELDWYP | SEQ ID |
| p40 subunit | DAPGEMVVLTCDTPEEDGITWTLDQSSEVLGSGKTLTIQV | NO: 46 |
| | KEFGDAGQYTCHKGGEVLSHSLLLLHKKEDGIWSTDILKD | |
| | QKEPKNKTFLRCEAKNYSGRFTCWWLTTISTDLTFSVKSS | |
| | RGSSDPQGVTCGAATLSAERVRGDNKEYEYSVECQEDSA | |
| | CPAAEESLPIEVMVDAVHKLKYENYTSSFFIRDIIKPDPPKN | |
| | LQLKPLKNSRQVEVSWEYPDTWSTPHSYFSLTFCVQVQG | |
| | KSKREKKDRVFTDKTSATVICRKNASISVRAQDRYYSSSW | |
| | SEWASVPCS | |
| Human IL-12 | ATGGGTCACCAGCAGTTGGTCATCTCTTGGTTTTCCCTGGTTT | SEQ ID |
| p40 subunit | TTCTGGCATCTCCCCTCGTGGCCATATGGGAACTGAAGAAAG | NO: 47 |
| | ATGTTTATGTCGTAGAATTGGATTGGTATCCGGATGCCCCTG | |
| | GAGAAATGGTGGTCCTCACCTGTGACACCCCTGAAGAAGAT | |
| | GGTATCACCTGGACCTTGGACCAGAGCAGTGAGGTCTTAGGC | |
| | TCTGGCAAAACCCTGACCATCCAAGTCAAAGAGTTTGGAGAT | |
| | GCTGGCCAGTACACCTGTCACAAAGGAGGCGAGGTTCTAAG | |
| | CCATTCGCTCCTGCTGCTTCACAAAAAGGAAGATGGAATTTG | |
| | GTCCACTGATATTTTAAAGGACCAGAAAGAACCCAAAAATA | |
| | AGACCTTTCTAAGATGCGAGGCCAAGAATTATTCTGGACGTT | |
| | TCACCTGCTGGTGGCTGACGACAATCAGTACTGATTTGACAT | |
| | TCAGTGTCAAAAGCAGCAGAGGCTCTTCTGACCCCCAAGGG | |
| | GTGACGTGCGGAGCTGCTACACTCTCTGCAGAGAGAGTCAG | |
| | AGGGGACAACAAGGAGTATGAGTACTCAGTGGAGTGCCAGG | |
| | AGGACAGTGCCTGCCCAGCTGCTGAGGAGAGTCTGCCCATTG | |
| | AGGTCATGGTGGATGCCGTTCACAAGCTCAAGTATGAAAACT | |
| | ACACCAGCAGCTTCTTCATCAGGGACATCATCAAACCTGACC | |
| | CACCCAAGAACTTGCAGCTGAAGCCATTAAAGAATTCTCGGC | |
| | AGGTGGAGGTCAGCTGGGAGTACCCTGACACCTGGAGTACT | |
| | CCACATTCCTACTTCTCCCTGACATTCTGCGTTCAGGTCCAGG | |
| | GCAAGAGCAAGAGAGAAAAGAAAGATAGAGTCTTCACGGAC | |
| | AAGACCTCAGCCACGGTCATCTGCCGCAAAAATGCCAGCATT | |
| | AGCGTGCGGGCCCAGGACCGCTACTATAGCTCATCTTGGAGC | |
| | GAATGGGCATCTGTGCCCTGCAGT | |
| Human IL-12 | RNLPVATPDPGMFPCLHHSQNLLRAVSNMLQKARQTLEF | SEQ ID |
| p35 subunit | YPCTSEEIDHEDITKDKTSTVEACLPLELTKNESCLNSRETS | NO: 48 |
| | FITNGSCLASRKTSFMMALCLSSIYEDSKMYQVEFKTMNA | |
| | KLLMDPKRQIFLDQNMLAVIDELMQALNFNSETVPQKSSL | |
| | EEPDFYKTKIKLCILLHAFRIRAVTIDRVMSYLNAS | |
| Human IL-12 | AGAAACCTCCCCGTGGCCACTCCAGACCCAGGAATGTT | SEQ ID |
| p35 subunit | CCCATGCCTTCACCACTCCCAAAACCTGCTGAGGGCCGT | NO: 49 |
| | CAGCAACATGCTCCAGAAGGCCAGACAAACTCTAGAAT | |
| | TTTACCCTTGCACTTCTGAAGAGATTGATCATGAAGATA | |
| | TCACAAAAGATAAAACCAGCACAGTGGAGGCCTGTTTA | |
| | CCATTGGAATTAACCAAGAATGAGAGTTGCCTAAATTC | |
| | CAGAGAGACCTCTTTCATAACTAATGGGAGTTGCCTGG | |
| | CCTCCAGAAAGACCTCTTTTATGATGGCCCTGTGCCTTA | |
| | GTAGTATTTATGAAGACTCGAAGATGTACCAGGTGGAG | |
| | TTCAAGACCATGAATGCAAAGCTTCTGATGGATCCTAA | |
| | GAGGCAGATCTTTCTAGATCAAAACATGCTGGCAGTTA | |
| | TTGATGAGCTGATGCAGGCCCTGAATTTCAACAGTGAG | |
| | ACTGTGCCACAAAAATCCTCCCTTGAAGAACCGGATTTT | |
| | TATAAAACTAAAATCAAGCTCTGCATACTTCTTCATGCT | |
| | TTCAGAATTCGGGCAGTGACTATTGATAGAGTGATGAG | |
| | CTATCTGAATGCTTCCTAA | |
| Human IL-15Ra | ATTACCTGCCCGCCGCCGATGAGCGTGGAACATGCGGA | SEQ ID |
| sushi domain | TATTTGGGTGAAAAGCTATAGCCTGTATAGCCGCGAAC | NO: 50 |
| | GCTATATTTGCAACAGCGGCTTTAAACGCAAAGCGGGC | |
| | ACCAGCAGCCTGACCGAATGCGTGCTGAACAAAGCGAC | |
| | CAACGTGGCGCATTGGACCACCCCGAGCCTGAAATGCA | |
| | TTCGCGATCCGGCGCTGGTGCATCAGCGCCCGGCGCCG | |
| | CCG | |
| Human IL-15 | ATGCGCATTAGCAAACCGCATCTGCGCAGCATTAGCAT | SEQ ID |
| | TCAGTGCTATCTGTGCCTGCTGCTGAACAGCCATTTTCT | NO: 51 |
| | GACCGAAGCGGGCATTCATGTGTTTATTCTGGGCTGCTT | |
| | TAGCGCGGGCCTGCCGAAAACCGAAGCGAACTGGGTGA | |
| | ACGTGATTAGCGATCTGAAAAAAATTGAAGATCTGATT | |
| | CAGAGCATGCATATTGATGCGACCCTGTATACCGAAAG | |
| | CGATGTGCATCCGAGCTGCAAAGTGACCGCGATGAAAT | |
| | GCTTTCTGCTGGAACTGCAGGTGATTAGCCTGGAAAGC | |
| | GGCGATGCGAGCATTCATGATACCGTGGAAAACCTGAT | |
| | TATTCTGGCGAACAACAGCCTGAGCAGCAACGGCAACG | |
| | TGACCGAAAGCGGCTGCAAAGAATGCGAAGAACTGGA | |
| | AGAAAAAAACATTAAAGAATTTCTGCAGAGCTTTGTGC | |
| | ATATTGTGCAGATGTTTATTAACACCAGC | |

TABLE 6
OTHER SEQUENCES

| | | |
|---|---|---|
| Linker | VPGXG, wherein X is any | SEQ ID |
| | amino acid except | NO: 22 |
| | proline | |
| Elastin-like | VPGXGVPGXG, wherein X | SEQ ID |
| polypeptide | is any amino acid | NO: 23 |
| sequence | except proline | |
| APMV-1 | G-R-Q-G-R↓L | SEQ ID |
| LaSota | | NO: 24 |
| APMV-2 | K-P-A-S-R↓F | SEQ ID |
| Yucaipa | | NO: 25 |
| APMV-3 | R-P-S-G-R↓L | SEQ ID |
| Wisconsin | | NO: 26 |
| APMV-4 | D-I-Q-P-R↓F | SEQ ID |
| Hong-Kong | | NO: 27 |
| APMV-6 | K-R-K-K-R↓F | SEQ ID |
| Hong-Kong | | NO: 28 |
| APMV-7 | L-P-S-S-R↓F | SEQ ID |
| Tennessee | | NO: 29 |
| APMV-8 | Y-P-Q-T-R↓L | SEQ ID |
| Delaware | | NO: 30 |
| APMV-9 | I-R-E-G-R↓I | SEQ ID |
| New York | | NO: 31 |
| Mlu I | ACGCGT | SEQ ID |
| restriction | | NO: 32 |
| site | | |
| Kozak | CCGCCACC | SEQ ID |
| sequence | | NO: 33 |
| Linker | GGGGGGS | SEQ ID |
| | | NO: 35 |
| Linker | SGGSGGGGSGGGSGGGGS | SEQ ID |
| | LQ | NO: 36 |
| Flag tag | DYKDDDDKIEGR | SEQ ID |
| | | NO: 38 |
| Signal | MAPRRARGCRTLGLPAL | SEQ ID |
| sequence | LLLLLLRPPATRG | NO: 41 |
| (IL-15 | | |
| signal | | |
| sequence) | | |
| Linker | AGCGGCGGCAGCGGCGGCG | SEQ ID |
| | GCGGCAGCGGCGGCGGCAG | NO: 42 |
| | CGGCGGCGGCGGCAGCCTG | |
| | CAG | |
| Signal | ATGGCGCCGCGCCGCGCGC | SEQ ID |
| sequence | GCGGCTGCCGCACCCTGGG | NO: 43 |
| | CCTGCCGGCGCTGCTGCTG | |
| | CTGCTGCTGCTGCGCCCGC | |
| | CGGCGACCCGCGGC | |
| Flag tag | GATTATAAAGATGATGATG | SEQ ID |
| | ATAAAATTGAAGGCCGC | NO: 44 |
| Linker | GGTGGCGGTGGCGGCGGAT | SEQ ID |
| | CT | NO: 45 |

### 6. EXAMPLE: ANTI-TUMOR PROPERTIES OF AVIAN PARAMYXOVIRUSES

This example demonstrates the efficacy of using APMV strains (especially, APMV-4 strains) to treat cancer. In particular, this example demonstrates that the use of APMV-4 Duck/Hong Kong/D3/1975 results in statistically significant anti-tumor activity in different animal models for various tumors.

### 6.1 Materials & Methods

### 6.1.1 Cell lines, Antibodies and Other Reagents

B16-F10 (mouse skin melanoma cells; ATCC Cat # CRL-6475, 2016), TC-1 (lung carcinoma; Johns Hopkins University, Baltimore, MD) and CT26 (murine colon carcinoma; ATCC Cat# CRL-2639, 2016) were maintained in DMEM or RPMI medium supplemented with 10% FBS (fetal bovine serum) and 2% penicillin and streptomycin). B16-F10, CT26 and TC-1 master cell-banks were created after purchase and early-passage cells were thawed in every experimental step. Once in culture, cells were maintained not longer than 8 weeks to guarantee genotypic stability and were monitored by microscopy. Required IMPACT test for in vivo experiments of the master-cell bank was performed by the Center for Comparative Medicine and Surgery at Icahn School of Medicine at Mt Sinai (Mount Sinai Hospital, New York, N.Y.). Reduced serum media Opti-MEM™ (Gibco™) was used as an in vitro viral infection medium. Rabbit polyclonal serum to NDV was previously described [14]. Avian paramyxovirus serotype-specific antiserums (type-2 471-ADV, type-3 473-ADV, type-4 475-ADV, type-6 479-ADV, type-7 481-ADV, type-8 483-ADV and type-9 485-ADV, 2017) were purchased from the National Veterinary Services Laboratories, United States Department of Agriculture (USDA, Ames, Iowa). Goat anti-chicken, Alexa-conjugated secondary antibody (Alexa-568, A-11041) was from Thermo Fisher. Hoechst 33258 nuclear staining reagent was purchased from Invitrogen (Molecular Probes, Eugene, Oreg.). CellTiter-Fluor™ cell viability assay (G608) was purchased from Promega.

### 6.1.2 Viruses

Modified Newcastle disease virus LaSota-L289A was generated in house and already tested as a therapeutic vector [43]. APMVs prototypes APMV-2 Chicken/California/Yucaipa/1956 (171ADV9701), APMV-3 Turkey/Wisconsin/1968 (173ADV9701), APMV-4 Duck/Hong Kong/D3/1975 (175ADV0601), APMV-6 Duck/Hong Kong/199/1977 (176ADV8101), APMV-7 Dove/Tennessee/4/1975(181ADV8101), APMV-8 Goose/Delaware/1053/1976 (none; 10/27/1986) and APMV-9 Duck/New york/22/1978 (185ADV 0301) were obtained from National Veterinary Services Laboratories, United States Department of Agriculture (USDA, Ames, Iowa). Viral stocks were propagated in 8 or 9 days embryonated chicken eggs and clear purified from the allantoic fluid. Viral titers were calculated by Hemagglutination assay (HA) using chicken blood (Lampire laboratories).

### 6.1.3 In Vitro Cell Viability Assay

B16-F10 cells were cultured at a confluence of 80% in 96 well dishes and infected at an MOI of 1 PFU/cell of the indicated virus. Viral suspension was removed lh post infection and cells were incubated in 100 μl of supplemented DMEM. 24 hours after infection, equal volume of the CellTiter-Fluor™ reagent (100 μl) was added to each well and cells were subsequently incubated 1 hour at 37° C. under restricted light conditions. The resulting fluorescence of each sample was recorded (400 nmEx/505 nmEmwavelength) using a Synergy H1 micro-plate reader (BioTek). Survival rate was calculated in reference to the viability of mock-infected cells (negative control). Survival rate (%)=[Fluor₅₀₅ₙₘ infected-sample/Fluor₅₀₅ₙₘ mock-infected sample]×100.

### 6.1.4 Fluorescence Microscopy

For indirect immunofluorescence staining, cells seeded in 96-well standard plates were infected for 1 h at an MOI of 1 PFU/cell in Opti-MEM™, after which the inoculum was removed and replaced with 100 μl of DMEM-FBS-P/S. At 20 hours post-infection cells were fixed with 2.5% paraformaldehyde for 15 minutes. Cell-membrane permeabilization was carried out using 0.2% Triton-PBS and blocked in PBS 1% BSA for 1 h. Primary antibodies were incubated with the samples for 1 h at room temperature. Secondary antibodies (goat anti-chicken Alexa Fluor 568, goat anti-rabbit Alexa Fluor 488; purchased from Invitrogen, USA) were used at a 1:1000 dilution for 45 minutes prior to imaging using an EVOS FL cell imagine system (Thermo Fisher).

### 6.1.5 Syngeneic Tumor Model

BALBc and C57/BL6J female mice 4-6 weeks of age used in all in vivo studies were purchased from Jackson Laboratory (Bar Harbor, ME). A B16-F10, TC-1 and CT26 cell suspension of 2.5×10⁵ cells (in 100 μl of PBS) was intradermally implanted into the flank of the right posterior leg of each C57B1/6 (melanoma and lung carcinoma) or BALBc (colon carcinoma) mouse. After 7-10 days, the mice were treated by intratumoral injection of 5×10⁶ PFU of the indicated virus or PBS. The intratumoral injections were administered every 24 hours for a total of four treatment doses. Tumor volume was monitored every 48 hours or every 24 hours when the last volume estimation was approaching the experimental endpoint of 1000 mm³. Mice were humanely euthanized the day in which the volume exceeded the predefined endpoint. Tumor measurement was determined using a digital caliper and total volume was calculated using the formula: Tumor volume (V)=L×W², where L, or tumor length, is the larger diameter, and W, or tumor width, is the smaller diameter.

### 6.1.6 Statistical Analysis

Statistical significance between results from triplicate samples was determined by one way-Anova (Dunnett's Multiple comparisons test). The results are expressed as mean value and standard deviations (SD). Comparative of survival curves for in syngeneic tumor models was performed using the long-rank (Mantel-Cox) test.

### 6.2 Results

### 6.2.1 Infectivity and Cytotoxicity of APMVs in B16-F10 Murine Melanoma Cancer Cell Line

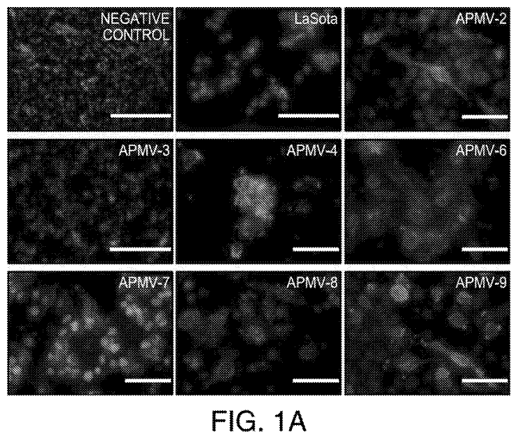

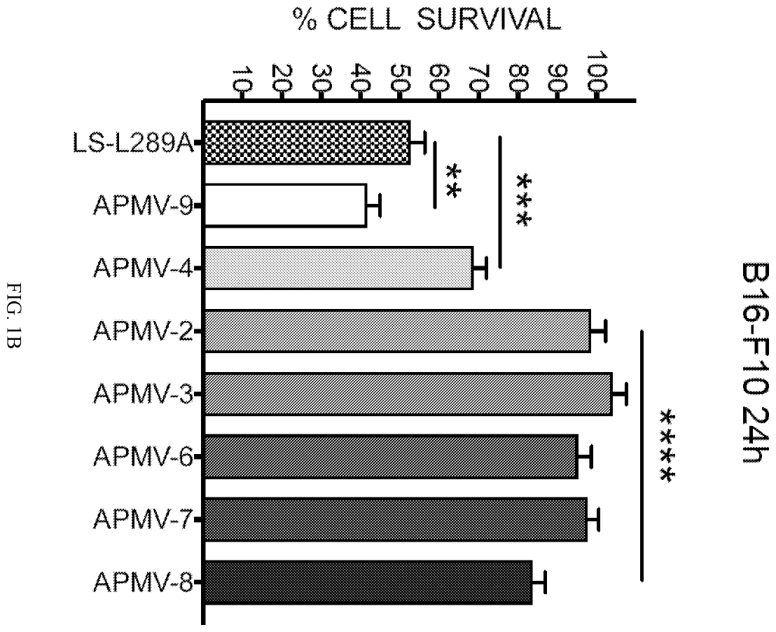

The capacity of the selected representative APMV strains (Table 4) to infect B16-F10 murine melanoma cancer cells was assessed. B16-F10 monolayers were exposed over 20 hours to a viral suspension containing 2×10⁵ ffu/ml of each of the chosen viruses (the equivalent to an MOI or multiplicity of infection of 1). The previously characterized lentogenic LaSota virus (APMV-1 serotype) was used as positive reference of infectivity and mock-infected cells were used as a negative control. After 20 hours of incubation, the samples were processed to detect the presence of viral antigens in infected cells by immunostaining. Positive fluorescence signal was detected in all the samples treated with the selected APMVs (FIG. 1A), demonstrating the susceptibility of the murine B16-F10 cancer cell line to be infected by avian avulaviruses other than NDV.

To evaluate the cytotoxic effect attained by the different serotypes, B16-F10 monolayers were infected at an MOI of 1 and incubated for 24 hours. Loss of viability was quantified as described above. Fluorometric analysis of the samples show that only APMV-9 and -4 prototypes were able to reduce cell viability to a similar extent as the LaSota virus, whereas the rest of the tested strains did not show relevant impact in cell viability at 24 hours after infection (FIG. 1B).

TABLE 4
APMV Serotypes and Prototype Viruses Included in the Study

| | | SEQUENCE | HA |
|---|---|---|---|
| SERO- | | ACCESSION | TI- |
| TYPE | STRAIN | NUMBER | TERS* |
| APMV-2 | Chicken/California/Yucaipa/1956 | EU338414.1 | 6-7 |
| APMV-3 | Turkey/Wisconsin/1968 | EU782025.1 | 7 |
| APMV-4 | Duck/Hong Kong/D3/1975 | FJ177514.1 | 7 |
| APMV-6 | Duck/Hong Kong/199/1977 | EU622637.2 | 7-8 |
| APMV-7 | Dove/Tennessee/4/1975 | FJ231524.1 | 8 |
| APMV-8 | Goose/Delaware/1053/1976 | FJ619036.1 | 7 |
| APMV-9 | Duck/New York/22/1978 | NC_025390.1 | 7-8 |

*Chicken red blood cells
Viruses were propagated in the allantoic cavity of embryonated, 8 days old, chicken eggs (SPF)

The pathogenicity in chickens of the selected APMVs included in the study are detailed in Table 5.

TABLE 5
Pathogenicity associated to the selected APMVS included in the study

| | F PROTEIN | |
|---|---|---|
| SEROTYPE | CLEAVAGE | |
| STRAIN | SITE | PATHOGENICITY IN CHICKENS |
| APMV-1 | G-R-Q-G-R↓L | Avirulent: no neurodegenerative disease, |
| LaSota | (SEQ ID NO: 24) | mild respiratory complications, |
| | | drop in egg production: Could grow |
| | | to 2¹⁰HA units in eggs. [84] |
| | | MDT: 112 h |
| | | ICP: 0 |
| APMV-2 | K-P-A-S-R↓F | Avirulent: no neurodegenerative disease |
| Yucaipa | (SEQ ID NO: 25) | (ICP in 1 day old chickens); mild |
| | | respiratory complications, drop in |
| | | egg production; Could grow to 2¹²HA |
| | | units in eggs. [85] |
| | | MDT > 168 h |
| | | ICP: 0 |
| APMV-3 | R-P-S-G-R↓L | No natural infections in chickens; |
| Wisconsin | (SEQ ID NO: 26) | Could grow to 2⁸HA units in 9 days |
| | | old eggs [86] |
| | | MDT > 168 h |
| | | ICP: 0 |
| APMV-4 | D-I-Q-P-R↓F | Avirulent; No disease in a day or |
| Hong-Kong | (SEQ ID NO: 27) | three-week-old chickens. Could growth |
| | | to high titers in eggs. [84] |
| | | MDT > 144 h |
| | | ICP: 0 |
| APMV-6 | K-R-K-K-R↓F | Avirulent. [84] |
| Hong-Kong | (SEQ ID NO: 28) | MDT > 168 h |
| | | ICP:0 |
| APMV-7 | L-P-S-S-R↓F | Av irulent. [84] |
| Tennessee | (SEQ ID NO: 29) | MDT > 144 h |
| | | ICP: 0 |
| APMV-8 | Y-P-Q-T-R↓L | Avirulent; Could grow to 2⁸HA units |
| Delaware | (SEQ ID NO: 30) | in eggs. [84] |
| | | MDT > 144 h |
| | | ICP: 0 |
| APMV-9 | I-R-E-G-R↓I | Avirulent: [84] |
| New York | (SEQ ID NO: 31) | MDT in eggs is more than 120 h |
| | | ICP: 0 |

MDT: Mean embryo Death Time is the mean time in hours for the minimal lethal dose to kill inoculated embryos. Virulent, 60 h; intermediate 60-90 h; avirulent > 90 h
ICP: Intracerebral pathogenicity index: evaluation of disease and death following intracerebral inoculation in 1-day-old SPF chicks. Virulent 1,5-2; intermediate 0.7-1.5; avirulent strains 0.7-0.0.

### 6.2.2 In Vivo Anti-Tumor Activities of APMVs in a Syngeneic Murine Melanoma Model

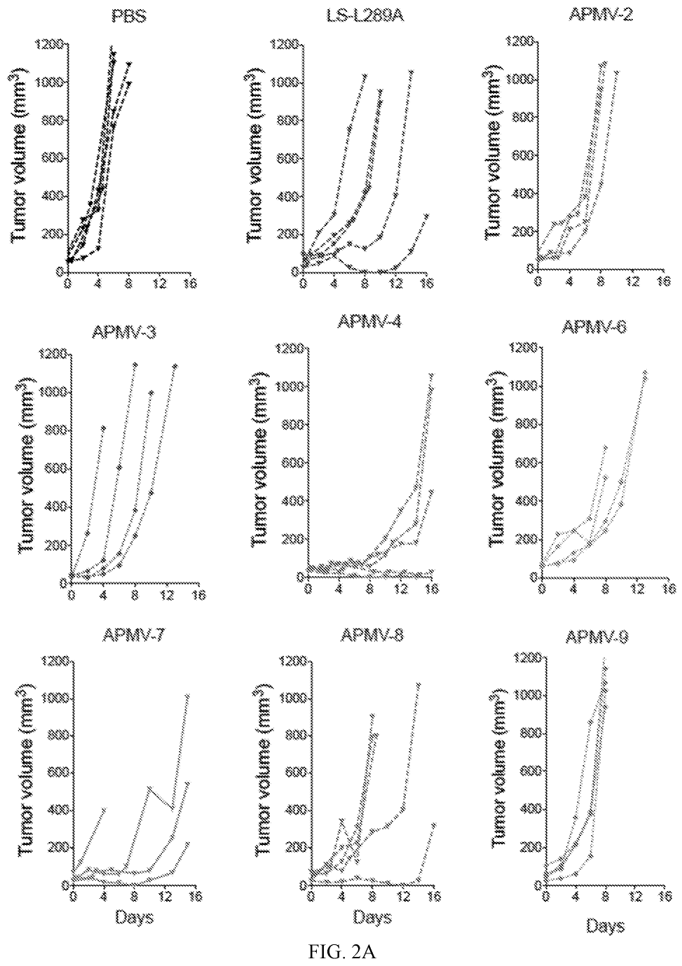

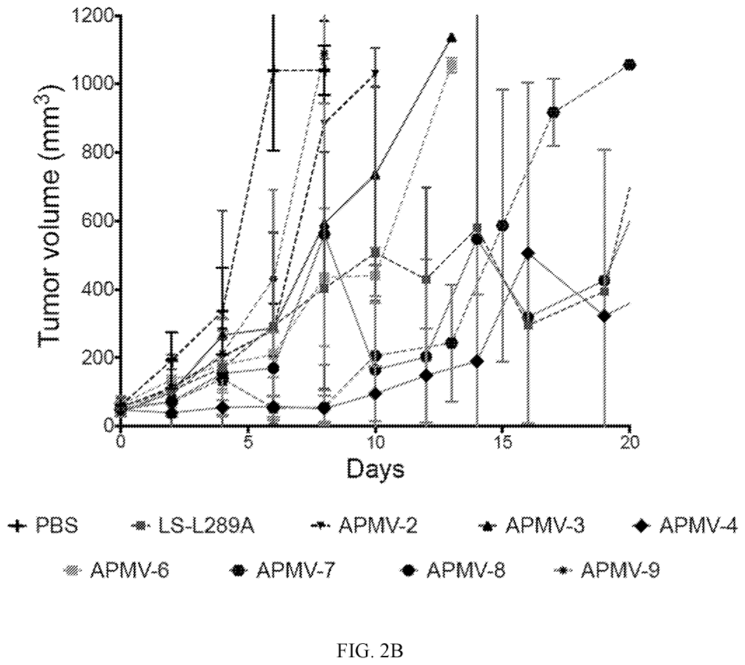

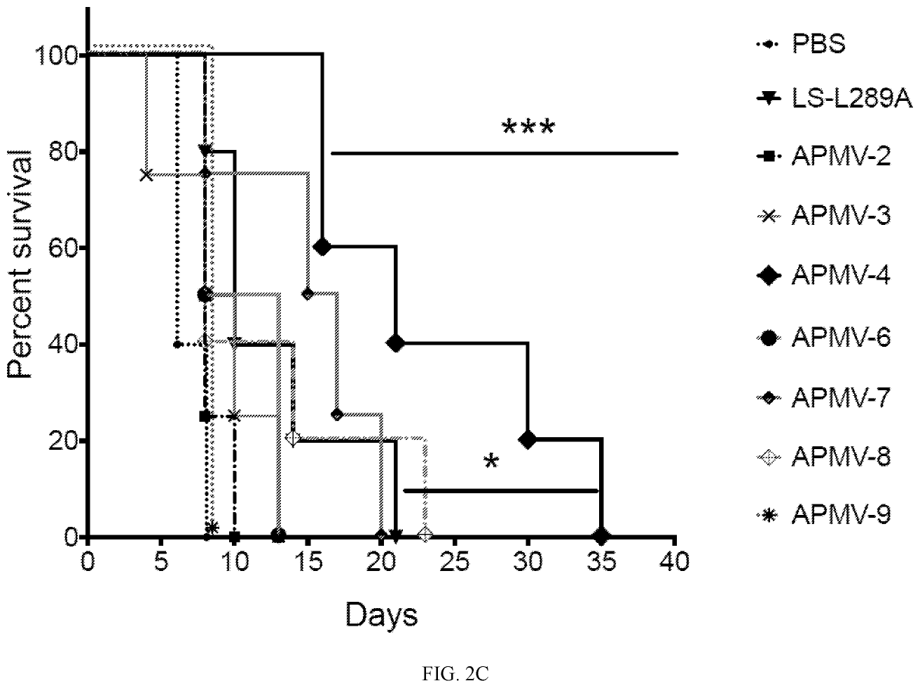

B16-F10 murine melanoma cells were intradermally implanted in the flank of the posterior right leg of C57BL/6 female mice. Tumors were allowed to develop for 10 days after which time the animals were intratumorally treated every other day with a total of four doses of 5×10⁶ PFU of La Sota-L289A or APMVs prototypes, or PBS for control mice (days 0, 2, 4 and 6; n=5 for each treatment group). The previously characterized LaSota-L289A virus (APMV-1 serotype) was used as positive reference of anti-tumor activity and a PBS mock-treated group was used as control of tumor growth. Tumor volume was monitored every 48 hours or every 24 hours when approaching the experimental end point of 1,000 mm³, after which mice were euthanized. FIG. 2A depicts tumor volume of individual mice at the indicated time points. FIG. 2B depicts the average tumor volume per experimental group at the indicated time points. Administration of the avulavirus prototypes controlled to some extent tumor growth early during treatment when compared to the PBS treated group, with the only exception being APMV-9. Only three of the avulavirus serotypes exerted prolonged anti-tumor activity: APMV -7, APMV-8, and APMV-4. APMV-7 and -8 treated groups showed delayed tumor growth and extended survival as compared to control at a similar rate as the reference LaSota-L289A virus. APMV-4 treated mice exhibited a profound inhibition in tumor growth and a statistically significant increase in survival time when compared to the reference LaSota-L289A virus (FIG. 2C). Error bars correspond to standard deviation of each group. (*, p<0.03).

### 6.2.3 Oncolytic Capacity of APMVs in a Syngeneic Murine Colon Carcinoma Model

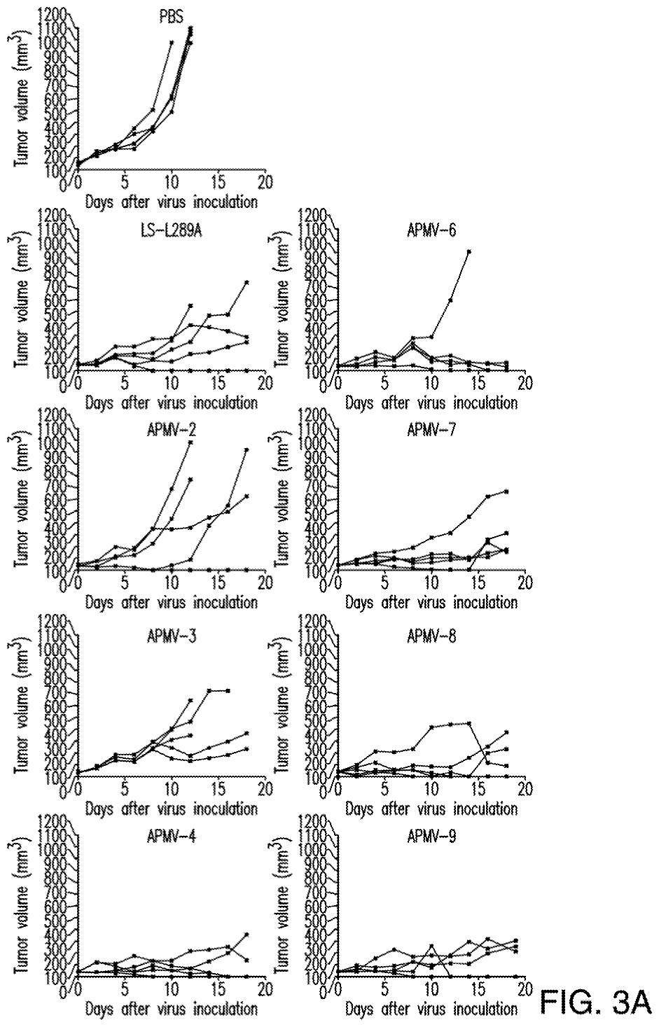

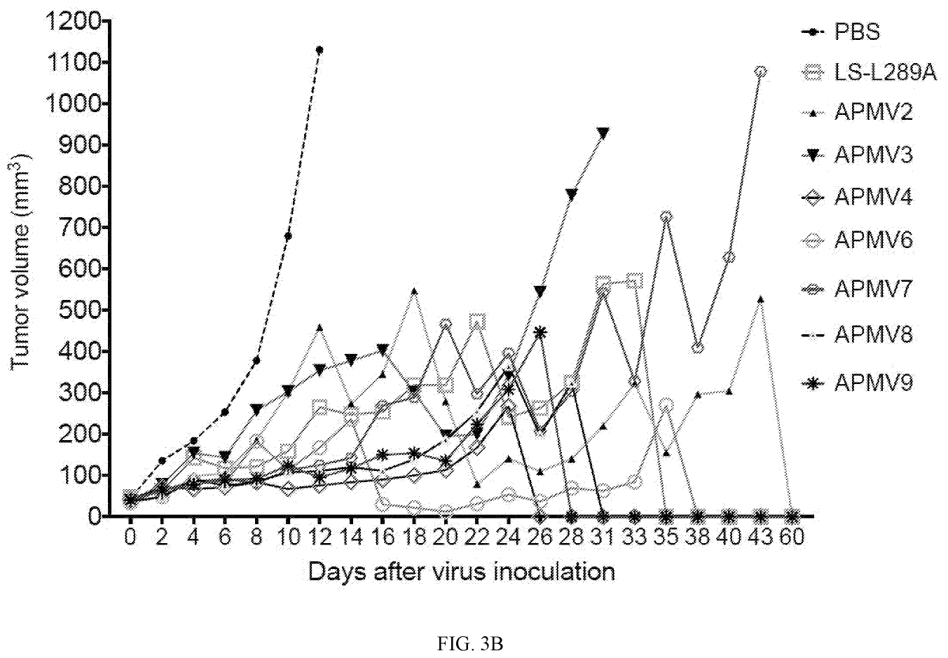

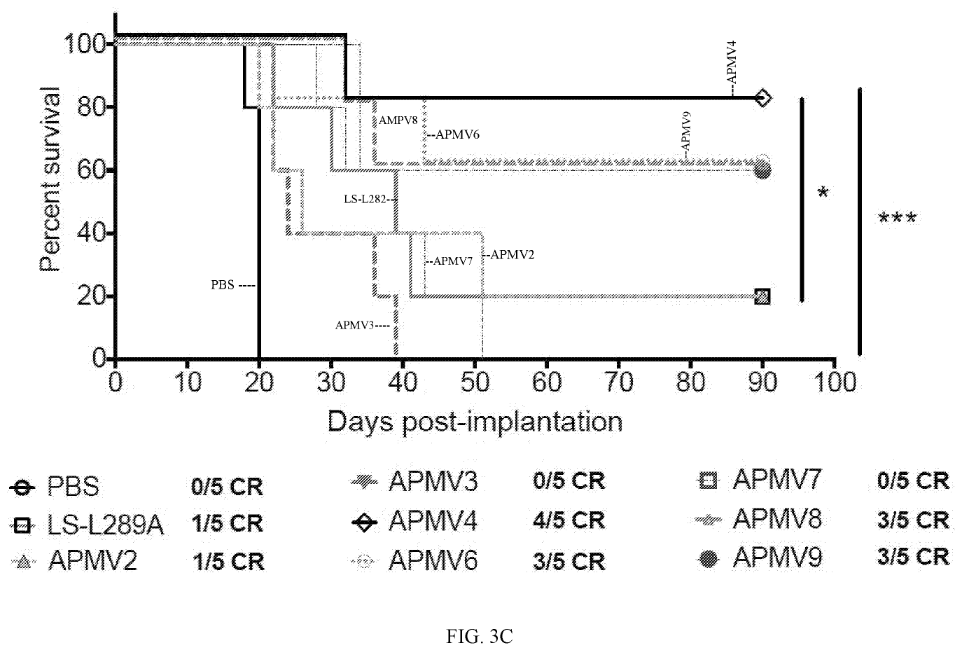

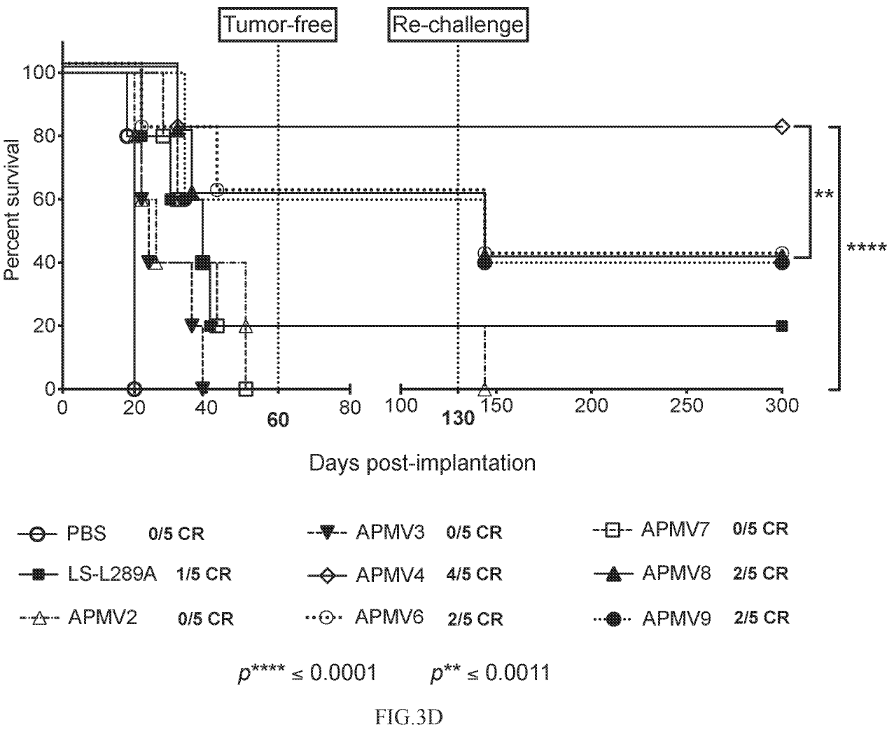

CT26 cells were implanted in the flank of the posterior right leg of BALBc mice. Starting on day 7 after tumor cell line injection, the animals were intratumorally treated every other day with a total of four doses of 5×10⁶ PFU of La Sota-L289A or APMVs prototypes, or PBS for control mice (days 0, 2, 4 and 6; n=5 for each treatment group). Tumor volume was monitored every 48 hours and then every 24 hours when approaching the experimental end point of 1,000 mm³, after which mice were euthanized. FIG. 3A depicts tumor growth of individual mice at the indicated time points. FIG. 3B depicts the average tumor volume of each treatment group at the indicated time points. Murine colon carcinoma was more susceptible to APMV induced-therapy than the melanoma model discussed above. All the APMV-treated groups exhibit a beneficial clinical response as demonstrated by the control of tumor growth and extended survival, when compared to the mock treated PBS group (FIGS. 3A and 3B). Furthermore, with the exception of APMV-3 and APMV-7, treatment with the selected APMV virus strains induced complete tumor remission (CR) in at least one animal in each treatment group. The APMV-4 and APMV-8 groups exhibited the best therapeutic response of the strains tested, where 4 out of 5 mice administered APMV-4 exhibited complete tumor remission and 3 out of 5 mice administered APMV-8 exhibited complete tumor remission (FIG. 3C).

On experimental day 130, tumor-free survivors were re-challenged by intradermal injection of 5×10⁵ CT26 cells in the flank of the posterior left leg (contralateral). As shown in FIG. 3D, APMV-4 re-challenged mice (4 out of 4) as well as LS-L289A′ single survivor displayed full protection against colon carcinoma development, which lasted for the extent of the long-term survival study (day 300). Contralateral tumor development was observed in 1 out of 3 of the re-challenge mice within the APMV-6, APMV-8 and APMV-9 experimental groups. No protection against re-challenge was observed in the APMV-2 treated group.

### 6.2.4 Oncolytic Capacity of APMV-4 in a Syngeneic Murine Lung Carcinoma Model

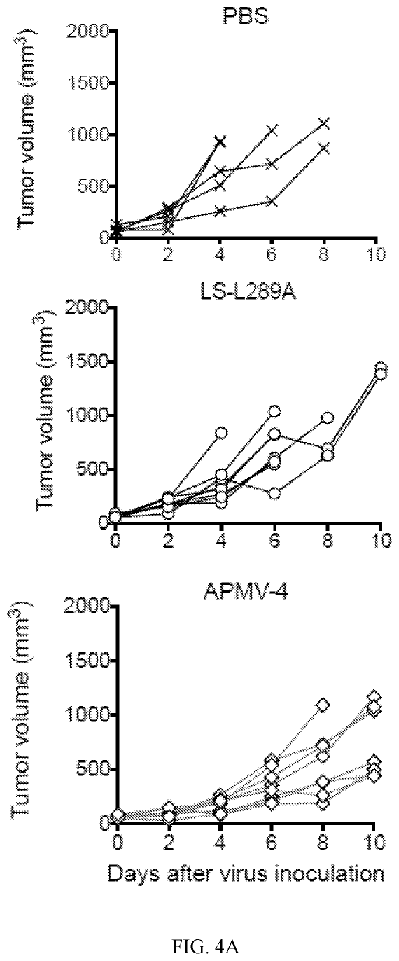

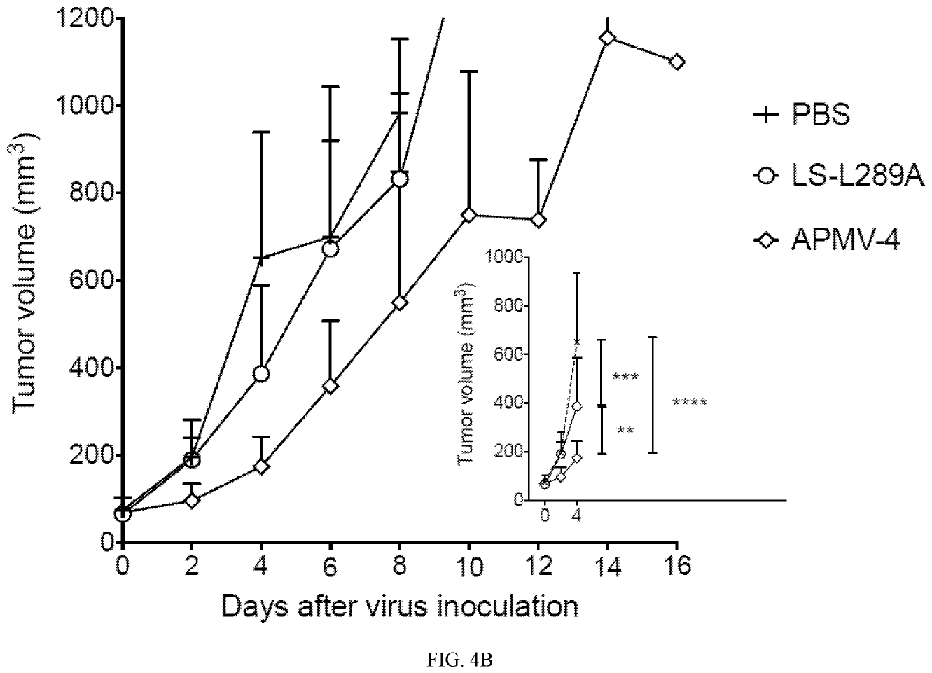

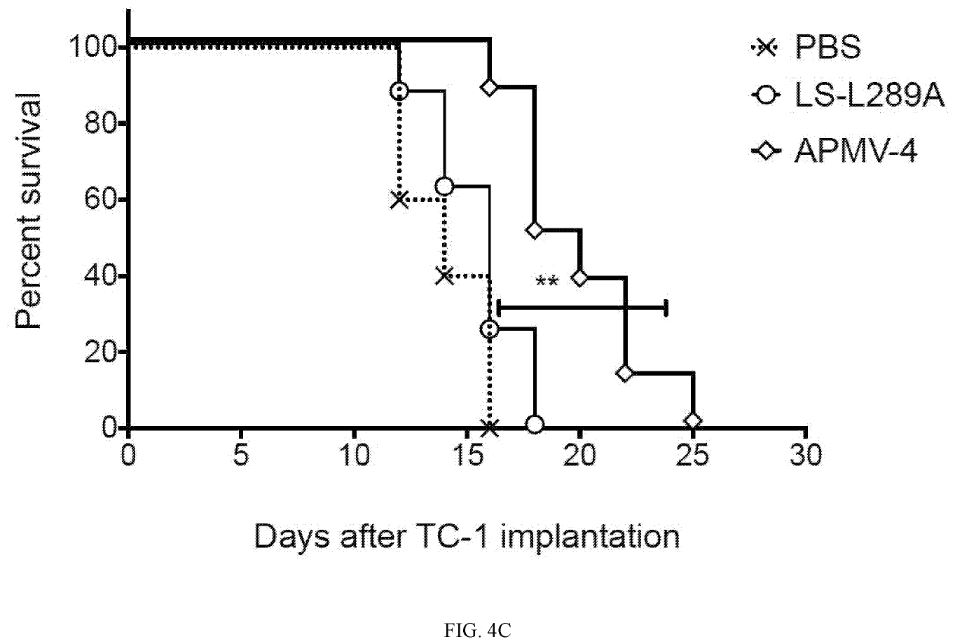

TC-1 cells were implanted in the flank of the posterior right leg of C57BL/6 mice. Starting on day 10 after tumor cell line injection, the animals were intratumorally treated every other day with a total of four doses of 5×10⁶ PFU of La Sota-L289A or APMV-4 Duck/Hong Kong/D3/1975, or PBS for control mice (days 0, 2, 4 and 6; n=5 for each treatment group). Tumor volume was monitored every 48 hours and then 24 hours when approaching the experimental end point of 1,000 mm³, at which time the mice were euthanized. FIG. 4A depicts tumor growth of individual mice at the indicated time points. FIG. 4B depicts the average tumor volume of each treatment group at the indicated time points. The overall survival of treated TC-1 tumor-bearing mice is shown in FIG. 4C (**, p<0.03). These data demonstrate that treatment with APMV-4 Duck/Hong Kong/D3/1975 strain results in enhanced antitumor response when compared to the LaSota-L289A APMV-1 strain and mock PBS treated groups. In this refractory tumor model, the response to APMV-4 oncolytic therapy features statistically significant control of tumor growth and prolonged survival.

### 6.2.5 References Cited in Background (Section 2) and Section 6

1. Lamb R A, & Parks, G. D. 2013. Paramyxoviridae: the viruses and their replication, 6th ed, vol 1. Lippincott, Williams, and Wilkins, Philadelphia.
2. Shnyrova A V, Ayllon J, Mikhalyov, II, Villar E, Zimmerberg J, Frolov V A. 2007. Vesicle formation by self-assembly of membrane-bound matrix proteins into a fluidlike budding domain. J Cell Biol 179:627-633.
3. Alexander D. 2003. Paramyxoviridae, 11th ed. Iowa State University Press, Iowa.
4. Afonso C L, Amarasinghe G K, Banyai K, Bao Y, Basler C F, Bavari S, Bejerman N, Blasdell K R, Briand F X, Briese T, Bukreyev A, Calisher C H, Chandran K, Cheng J, Clawson A N, Collins P L, Dietzgen R G, Dolnik O, Domier L L, Durrwald R, Dye J M, Easton A J, Ebihara H, Farkas S L, Freitas-Astua J, Formenty P, Fouchier R A, Fu Y, Ghedin E, Goodin M M, Hewson R, Hone M, Hyndman T H, Jiang D, Kitajima E W, Kobinger G P, Kondo H, Kurath G, Lamb R A, Lenardon S, Leroy E M, Li CX, Lin X D, Liu L, Longdon B, Marton S, Maisner A, Muhlberger E, Netesov S V, Nowotny N, et al. 2016. Taxonomy of the order Mononegavirales: update 2016. Arch Virol, 161:2351-2360.
5. Gogoi P, Ganar K, Kumar S. 2017. Avian Paramyxovirus: A Brief Review. Transbound Emerg Dis 64:53-67.
6. Hines N L, Miller C L. 2012. Avian paramyxovirus serotype-1: a review of disease distribution, clinical symptoms, and laboratory diagnostics. Vet Med Int 2012:708216.
7. Ganar K, Das M, Sinha S, Kumar S. 2014. Newcastle disease virus: current status and our understanding. Virus Res 184:71-81.
8. Senne D A, King D J, Kapczynski D R. 2004. Control of Newcastle disease by vaccination. Dev Biol (Basel) 119:165-170.
9. Dortmans J C, Peeters B P, Koch G. 2012. Newcastle disease virus outbreaks: vaccine mismatch or inadequate application? Vet Microbiol 160:17-22.
10. Dortmans J C, Koch G, Rottier P J, Peeters B P. 2011. Virulence of Newcastle disease virus: what is known so far? Vet Res 42:122.
11. Elmberg J, Berg C, Lerner H, Waldenstrom J, Hessel R. 2017. Potential disease transmission from wild geese and swans to livestock, poultry and humans: a review of the scientific literature from a One Health perspective. Infect Ecol Epidemiol 7:1300450.
12. Park M S, Shaw M L, Munoz-Jordan J, Cros J F, Nakaya T, Bouvier N, Palese P, Garcia-Sastre A, Basler C F. 2003. Newcastle disease virus (NDV)-based assay demonstrates interferon-antagonist activity for the NDV V protein and the Nipah virus V, W, and C proteins. J Virol 77:1501-1511.
13. Wilden H, Fournier P, Zawatzky R, Schirrmacher V. 2009. Expression of RIG-I, IRF3, IFN-beta and IRF7 determines resistance or susceptibility of cells to infection by Newcastle Disease Virus. Int J Oncol 34:971-982.
14. Park M S, Garcia-Sastre A, Cros J F, Basler C F, Palese P. 2003. Newcastle disease virus V protein is a determinant of host range restriction. J Virol 77:9522-9532.
15. Jarahian M, Watzl C, Fournier P, Arnold A, Djandji D, Zahedi S, Cerwenka A, Paschen A, Schirrmacher V, Momburg F. 2009. Activation of natural killer cells by newcastle disease virus hemagglutinin-neuraminidase. J Virol 83:8108-8121.
16. Ginting T E, Suryatenggara J, Christian S, Mathew G. 2017. Proinflammatory response induced by Newcastle disease virus in tumor and normal cells. Oncolytic Virother 6:21-30.
17. Schirrmacher V, Fournier P. 2009. Newcastle disease virus: a promising vector for viral therapy, immune therapy, and gene therapy of cancer. Methods Mol Biol 542:565-605.
18. Kapczynski D R, Afonso C L, Miller P J. 2013. Immune responses of poultry to Newcastle disease virus. Dev Comp Immunol 41:447-453.
19. Schirrmacher V, Ahlert T, Probstle T, Steiner H H, Herold-Mende C, Gerhards R, Hagmuller E, Steiner H H. 1998. Immunization with virus-modified tumor cells. Semin Oncol 25:677-696.
20. Romer-Oberdorfer A, Mundt E, Mebatsion T, Buchholz U J, Mettenleiter T C. 1999. Generation of recombinant lentogenic Newcastle disease virus from cDNA. J Gen Virol 80 (Pt 11):2987-2995.
21. Peeters B P, de Leeuw O S, Koch G, Gielkens A L. 1999. Rescue of Newcastle disease virus from cloned cDNA: evidence that cleavability of the fusion protein is a major determinant for virulence. J Virol 73:5001-5009.
22. Nakaya T, Cros J, Park M-S, Nakaya Y, Zheng H, Sagrera A, Villar E, Garcia-Sastre A, Palese P. 2001. Recombinant Newcastle disease virus as a vaccine vector. J Virol 75:11868-11873.
23. Maamary J, Array F, Gao Q, Garcia-Sastre A, Steinman R M, Palese P, Nchinda G. 2011. Newcastle disease virus expressing a dendritic cell-targeted HIV gag protein induces a potent gag-specific immune response in mice. J Virol 85:2235-2246.
24. Park M S, Steel J, Garcia-Sastre A, Swayne D, Palese P. 2006. Engineered viral vaccine constructs with dual specificity: avian influenza and Newcastle disease. Proc Natl Acad Sci USA 103:8203-8208.
25. Swayne D E, Suarez D L, Schultz-Cherry S, Tumpey T M, King D J, Nakaya T, Palese P, Garcia-Sastre A. 2003. Recombinant paramyxovirus type 1-avian influenza-H7 virus as a vaccine for protection of chickens against influenza and Newcastle disease. Avian Dis 47:1047-1050.
26. Martinez-Sobrido L, Gitiban N, Fernandez-Sesma A, Cros J, Mertz S E, Jewell N A, Hammond S, Flano E, Durbin R K, Garcia-Sastre A, Durbin J E. 2006. Protection against respiratory syncytial virus by a recombinant Newcastle disease virus vector. J Virol 80:1130-1139.
27. Fournier P, Arnold A, Schirrmacher V. 2009. Polarization of human monocyte-derived dendritic cells to DC1 by in vitro stimulation with Newcastle Disease Virus. J BUON 14 Suppl 1:S111-122.
28. Carnero E, Li W, Borderia A V, Moltedo B, Moran T, Garcia-Sastre A. 2009. Optimization of human immunodeficiency virus gag expression by newcastle disease virus vectors for the induction of potent immune responses. J Virol 83:584-597.
29. Schirrmacher V. 2016. Fifty Years of Clinical Application of Newcastle Disease Virus: Time to Celebrate! Biomedicines 4.
30. Cuadrado-Castano S, Sanchez-Aparicio M T, Garcia-Sastre A, Villar E. 2015. The therapeutic effect of death: Newcastle disease virus and its antitumor potential. Virus Res 209:56-66.
31. Fiola C, Peeters B, Fournier P, Arnold A, Bucur M, Schirrmacher V. 2006. Tumor selective replication of Newcastle disease virus: association with defects of tumor cells in antiviral defence. Int J Cancer 119:328-338.
32. Washburn B, Schirrmacher V. 2002. Human tumor cell infection by Newcastle Disease Virus leads to upregulation of HLA and cell adhesion molecules and to induction of interferons, chemokines and finally apoptosis. Int J Oncol 21:85-93.
33. Lam H Y, Yeap S K, Rasoli M, Omar A R, Yusoff K, Suraini A A, Alitheen N B. 2011. Safety and clinical usage of newcastle disease virus in cancer therapy. J Biomed Biotechnol 2011:718710.
34. Schirrmacher V, Haas C, Bonifer R, Ahlert T, Gerhards R, Ertel C. 1999. Human tumor cell modification by virus infection: an efficient and safe way to produce cancer vaccine with pleiotropic immune stimulatory properties when using Newcastle disease virus. Gene Ther 6:63-73.
35. Cassel W A, Garrett R E. 1965. Newcastle Disease Virus as an Antineoplastic Agent. Cancer 18:863-868.
36. Wheelock E F, Dingle J H. 1964. Observations on the Repeated Administration of Viruses to a Patient with Acute Leukemia. A Preliminary Report. N Engl J Med 271:645-651.
37. Pecora A L, Rizvi N, Cohen G I, Meropol N J, Sterman D, Marshall J L, Goldberg S, Gross P, O'Neil J D, Groene W S, Roberts M S, Rabin H, Bamat M K, Lorence R M. 2002. Phase I trial of intravenous administration of PV701, an oncolytic virus, in patients with advanced solid cancers. J Clin Oncol 20:2251-2266.
38. Csatary L K, Gosztonyi G, Szeberenyi J, Fabian Z, Liszka V, Bodey B, Csatary C M. 2004. MTH-68/H oncolytic viral treatment in human high-grade gliomas. J Neurooncol 67:83-93.
39. Freeman A I, Zakay-Rones Z, Gomori J M, Linetsky E, Rasooly L, Greenbaum E, Rozenman-Yair S, Panet A, Libson E, Irving C S, Galun E, Siegal T. 2006. Phase I/II trial of intravenous NDV-HUJ oncolytic virus in recurrent glioblastoma multiforme. Mol Ther 13:221-228.
40. Heicappell R, Schirrmacher V, von Hoegen P, Ahlert T, Appelhans B. 1986. Prevention of metastatic spread by postoperative immunotherapy with virally modified autologous tumor cells. I. Parameters for optimal therapeutic effects. Int J Cancer 37:569-577.
41. Lorence R M, Rood P A, Kelley K W. 1988. Newcastle disease virus as an antineoplastic agent: induction of tumor necrosis factor-alpha and augmentation of its cytotoxicity. J Natl Cancer Inst 80:1305-1312.
42. Steiner H H, Bonsanto M M, Beckhove P, Brysch M, Geletneky K, Ahmadi R, Schuele-Freyer R, Kremer P, Ranaie G, Matejic D, Bauer H, Kiessling M, Kunze S, Schirrmacher V, Herold-Mende C. 2004. Antitumor vaccination of patients with glioblastoma multiforme: a pilot study to assess feasibility, safety, and clinical benefit. J Clin Oncol 22:4272-4281.
43. Liang W, Wang H, Sun T M, Yao W Q, Chen L L, Jin Y, Li C L, Meng F J. 2003. Application of autologous tumor cell vaccine and NDV vaccine in treatment of tumors of digestive tract. World J Gastroenterol 9:495-498.
44. Karcher J, Dyckhoff G, Beckhove P, Reisser C, Brysch M, Ziouta Y, Helmke B H, Weidauer H, Schirrmacher V, Herold-Mende C. 2004. Antitumor vaccination in patients with head and neck squamous cell carcinomas with autologous virus-modified tumor cells. Cancer Res 64:8057-8061.
45. Pomer S, Schirrmacher V, Thiele R, Lohrke H, Brkovic D, Staehler G. 1995. Tumor response and 4 year survival-data of patients with advanced renal-cell carcinoma treated with autologous tumor vaccine and subcutaneous R-IL-2 and IFN-alpha(2b). Int J Oncol 6:947-954.
46. Bohle W, Schlag P, Liebrich W, Hohenberger P, Manasterski M, Moller P, Schirrmacher V. 1990. Postoperative active specific immunization in colorectal cancer patients with virus-modified autologous tumor-cell vaccine. First clinical results with tumor-cell vaccines modified with live but avirulent Newcastle disease virus. Cancer 66:1517-1523.
47. Bai L, Koopmann J, Fiola C, Fournier P, Schirrmacher V. 2002. Dendritic cells pulsed with viral oncolysates potently stimulate autologous T cells from cancer patients. Int J Oncol 21:685-694.
48. Schirrmacher V, Fournier P. 2014. Multimodal cancer therapy involving oncolytic Newcastle disease virus, autologous immune cells, and bi-specific antibodies. Front Oncol 4:224.
49. Schirrmacher V, Bihari A S, Stucker W, Sprenger T. 2014. Long-term remission of prostate cancer with extensive bone metastases upon immuno- and virotherapy: A case report. Oncol Lett 8:2403-2406.
50. Zamarin D, Holmgaard R B, Subudhi S K, Park J S, Mansour M, Palese P, Merghoub T, Wolchok J D, Allison J P. 2014. Localized oncolytic virotherapy overcomes systemic tumor resistance to immune checkpoint blockade immunotherapy. Sci Transl Med 6:226ra232.
51. Zamarin D, Holmgaard R B, Ricca J, Plitt T, Palese P, Sharma P, Merghoub T, Wolchok J D, Allison J P. 2017. Intratumoral modulation of the inducible co-stimulator ICOS by recombinant oncolytic virus promotes systemic anti-tumour immunity. Nat Commun 8:14340.
52. Li P, Chen C H, Li S, Givi B, Yu Z, Zamarin D, Palese P, Fong Y, Wong R J. 2011. Therapeutic effects of a fusogenic newcastle disease virus in treating head and neck cancer. Head Neck 33:1394-1399.
53. Zamarin D, Palese P. 2012. Oncolytic Newcastle disease virus for cancer therapy: old challenges and new directions. Future Microbiol 7:347-367.
54. Cuadrado-Castano S, Ayllon J, Mansour M, de la Iglesia-Vicente J, Jordan S, Tripathi S, Garcia-Sastre A, Villar E. 2015. Enhancement of the proapoptotic properties of newcastle disease virus promotes tumor remission in syngeneic murine cancer models. Mol Cancer Ther 14:1247-1258.
55. Zamarin D, Vigil A, Kelly K, Garcia-Sastre A, Fong Y. 2009. Genetically engineered Newcastle disease virus for malignant melanoma therapy. Gene Ther 16:796-804.
56. Zamarin D, Martinez-Sobrido L, Kelly K, Mansour M, Sheng G, Vigil A, Garcia-Sastre A, Palese P, Fong Y. 2009. Enhancement of oncolytic properties of recombinant newcastle disease virus through antagonism of cellular innate immune responses. Mol Ther 17:697-706.
57. Zhao H, Janke M, Fournier P, Schirrmacher V. 2008. Recombinant Newcastle disease virus expressing human interleukin-2 serves as a potential candidate for tumor therapy. Virus Res 136:75-80.
58. Vigil A, Martinez O, Chua M A, Garcia-Sastre A. 2008. Recombinant Newcastle disease virus as a vaccine vector for cancer therapy. Mol Ther 16:1883-1890.
59. Vigil A, Park M S, Martinez O, Chua M A, Xiao S, Cros J F, Martinez-Sobrido L, Woo S L, Garcia-Sastre A. 2007. Use of reverse genetics to enhance the oncolytic properties of Newcastle disease virus. Cancer Res 67:8285-8292.
60. Sergel T A, McGinnes L W, Morrison T G. 2000. A single amino acid change in the Newcastle disease virus fusion protein alters the requirement for HN protein in fusion. J Virol 74:5101-5107.
61. Doyle, T., 1927:A hitherto unrecorded disease of fowls due to a filter-passing virus. J. Comp. Pathol. Ther. 40,144-169.
62. Bankowski, R. A., J. Almquist and J. Dombrucki, 1981: Effect of paramyxovirus Yucaipa on fertility, hatchability, and poult yield of turkeys. AvianDis. 25, 517-520.
63. Tumova, B., J. H. Robinson, and B. C. Easterday, 1979: A hitherto unreported paramyxovirus of turkeys. Res. Vet. Sci. 27,135-140.
64. Andral, B., and D. Toquin, 1984: Isolation of avian paramyxovirus 2 and 3 from turkeys in Brittany. Vet. Rec. 114,570-571.
65. Alexander, D. J., and N. J. Chettle, 1978: Relationship of parakeet/Netherlands/449/75 virus to other avianparamyxovirus-es. Res. Vet. Sci. 25,105-106.
66. Webster, R. G., M. Morita, C. Pridgen and B. Tumova, 1976: Ortho-and paramyxoviruses from migrating feral ducks: characterization of a new group of influenza A viruses. J. Gen. Virol. 32,217-225.
67. Abolnik, C., M. de Castro and J. Rees, 2012: Full genomic sequence of an African avian paramyxovirus type 4 strain isolated from a wild duck. VirusGenes 45,537-541.
68. Mustaffa Babjee, A., P. B. Spradbrow and J. L. Samuel, 1974: A pathogenic paramyxovirus from a budgerigar (Melopsittacus undulatus). AvianDis. 18, 226-230.
69. Boisseau, J., 1993: Basis for the evaluation of the microbiological risks due to veterinary drug residues in food. Vet. Microbiol. 35,187-192.
70. Shortridge, K. F., D. J. Alexander, and M. S. Collins, 1980: Isolation and properties of viruses from poultry in HongKong which represent a new (sixth) distinct group of avian para-myxoviruses. J. Gen. Virol. 49,255-262.
71. Stanislawek, W. L., C. R. Wilks, J. Meers, G. W. Horner, D. J. Alexander, R. J. Manvell, J. A. Kattenbelt and A. R. Gould, 2002: Avian paramyxoviruses and influenza viruses isolated from mallard ducks (Anasplatyrhynchos) in New Zealand. Arch. Virol. 147,1287-1302.
72. Alexander, D. J., V. S. Hinshaw and M. S. Collins, 1981: Characterization of viruses from doves representing a new serotype of avian paramyxoviruses. Arch. Virol.68,265-269.
73. Saif, Y. M., R. Mohan, L. Ward, D. A. Senne, B. Panigrahy and R. N. Dearth, 1997: Natural and experimental infection of turkeys with avian paramyxovirus-7. AvianDis. 41,326-329.
74. Woolcock, P. R., J. D. Moore, M. D. McFarland and B. Panigrahy, 1996: Isolation of paramyxovirus serotype 7 from ostriches(Struthiocamelus). AvianDis.40,945-949.
75. Yamane, N., J. Arikawa, T. Odagiri and N. Ishida, 1982: Characterization of avian paramyxoviruses isolated from feral ducks in northern Japan: the presence of three distinct viruses innature. Microbiol. Immunol. 26,557-568.
76. Cloud, S., and J. Rosenberger, 1980: Characterization of nine avian paramyxoviruses. AvianDis. 24,139-152.
77. Capua, I., R. DeNardi, M. S. Beato, C. Terregino, M. Scremin and V. Guberti, 2004: Isolation of an avian paramyxovirus type 9 from migratory waterfowl in Italy. Vet. Rec. 155,156.
78. Sandhu, T. and V. Hinshaw, 1981: Influenza A virus infection of domestic ducks. AvianDis. 47,93-99.
79. Miller, P. J., C. L. Afonso, E. Spackman, M. A. Scott, J. C. Pedersen, D. A. Senne, J. D. Brown, C. M. Fuller, M. M. Uhart, W. B. Karesh, I. H. Brown, D. J. Alexander and D. E. Swayne, 2010: Evidence for a new avian paramyxovirus serotype 10 detected in rockhopper penguins from the Falkland Islands. J.Virol. 84,11496-11504.
80. Briand, F. X., A. Henry, P. Massin and V. Jestin, 2012: Complete genome sequence of a novel avian paramyxovirus. J. Virol. 86,7710.
81. Terregino, C., E. W. Aldous, A. Heidari, C. M. Fuller, R. DeNardi, R. J. Manvell, M. S. Beato, W. M. Shell, I. Monne, I. H. Brown, D. J. Alexander and I. Capua, 2013: Antigenic and genetic analyses of isolate APMV/wigeon/Italy/3920-1/2005 indicate that it represents a new avian paramyxovirus (APMV-12). Arch. Virol. 158,2233-2243.
82. Yamamoto, E., Ito, H., Tomioka, Y. and Ito, T., 2015: Characterization of novel avian paramyxovirus strain APMV/Shimane67 isolated from migratory wild geese in Japan. Journal of Veterinary Medical Science, 77(9), 1079-1085.
83. Karamendin, K., Kydyrmanov, A., Seidalina, A., Asanova, S., Sayatov, M., Kasymbekov, E., Zhumatov, K., 2016: Complete Genome Sequence of a Novel Avian Paramyxovirus (APMV-13) Isolated from a Wild Bird in Kazakhstan. Genome Announcements, 4(3), e00167-16.
84. Kim S H, Xiao S, Shive H, Collins P L, Samal S K., 2012: Replication, Neurotropism, and Pathogenicity of Avian Paramyxovirus Serotypes 1-9 in Chickens and Ducks. PLoS ONE:7(4):e34927.
85. Subbiah, M., Xiao, S., Khattar, S. K., Dias, F. M., Collins, P. L., & Samal, S. K., 2010: Pathogenesis of two strains of Avian Paramyxovirus serotype 2, Yucaipa and Bangor, in chickens and turkeys. Avian Diseases, 54(3), 1050-1057.
86. Kumar S, Militino Dias F, Nayak B, Collins P L, Samal S. K., 2010: Experimental avian paramyxovirus serotype-3 infection in chickens and turkeys. Veterinary Research.;41(5):72.

### 7. DEVELOPMENT OF RECOMBINANT APMV-4 ENCODING HUMAN IL-12

The nucleotide sequence CATCGA (SEQ ID NO:52) in the P-M intergenic region of APMV-4/Duck/Hong Kong/D3/1975 strain (residues 2932-2938 of the cDNA sequence of the APMV-4 genome) is altered to form the Mlu I restriction site (ACGCGT (SEQ ID NO:32)). A transgene comprising a Mlu I restriction site, a Kozak sequence (CCGCCACC (SEQ ID NO:33)), a nucleotide sequence encoding human IL-12 protein (e.g., a transgene comprising the nucleotide sequence of SEQ ID NO:16 or 17), and nucleotides CCC is inserted between the P and M genes (the P-M intergenic region; 34 nt from 2979 to 3013) of the APMV-4 strain. As a result of performing this methodology using SEQ ID NO:16 for the nucleotide sequence encoding IL-12 protein, a recombinant APMV-4 comprising a packaged genome is produced. In particular, the recombinant APMV-4-hIL-12 comprising a packaged genome is produced, wherein the packaged genome comprises (or consists of) the negative sense RNA transcribed from the cDNA sequence set forth in SEQ ID NO:14.

### 8. EMBODIMENTS

Provided herein are the following exemplary embodiments:

1. A method for treating cancer, comprising administering to a human subject in need thereof a naturally occurring avian paramyxovirus serotype 4 (APMV-4), wherein the APMV-4 has an intracerebral pathogenicity index in day-old chicks of the Gallus gallus species of less than 0.7.

2. A method for treating cancer, comprising administering to a human subject in need thereof a recombinant APMV-4, wherein the recombinant APMV-4 has an intracerebral pathogenicity index in day-old chicks of the Gallus gallus species of less than 0.7.

3. The method of embodiment 1 or 2, wherein administration of the APMV-4 decreases tumor growth and increases survival in a B16-F10 syngeneic murine melanoma model as compared to tumor growth and survival in B16-F10 syngeneic murine melanoma model administered phosphate buffered saline (PBS).

4. The method of embodiment 1 or 2, wherein administration of the APMV-4 results in a greater decrease in tumor growth and a longer survival time in a B16-F10 syngeneic murine melanoma model as compared to tumor growth and survival time in a B16-F10 syngeneic murine melanoma model administered a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A.

5. The method of embodiment 4, wherein the packaged genome of the modified NDV LaSota comprises the negative sense RNA transcribed from the cDNA sequence set forth in SEQ ID NO:13.

6. The method of embodiment 1 or 2, wherein administration of the APMV-4 decreases tumor growth and increases survival in a BALBc syngeneic murine colon carcinoma tumor model as compared to tumor growth and survival in BALBc syngeneic murine colon carcinoma tumor model administered phosphate buffered saline (PBS).

7. The method of embodiment 1 or 2, wherein administration of the APMV-4 results in a greater decrease in tumor growth and a longer survival time in a BALBc syngeneic murine colon carcinoma tumor model as compared to tumor growth and survival time in the BALBc syngeneic murine colon carcinoma tumor model administrated a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A.

8. The method of embodiment 7, wherein the packaged genome of the modified NDV LaSota comprises the negative sense RNA transcribed from the cDNA sequence set forth in SEQ ID NO:13.

9. The method of embodiment 1 or 2, wherein administration of the APMV-4 decreases tumor growth and increases survival in a C57BL/6 syngeneic murine lung carcinoma tumor model as compared to tumor growth and survival in a C57BL/6 syngeneic murine lung carcinoma tumor model administered phosphate buffered saline (PBS).

10. The method of embodiment 1 or 2, wherein administration of the APMV-4 results in a greater decrease in tumor growth and a longer survival time in a C57BL/6 syngeneic murine lung carcinoma tumor model as compared to tumor growth and survival time in a C57BL/6 syngeneic murine lung carcinoma tumor model administered a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A.

11. The method of embodiment 10, wherein the packaged genome of the modified NDV LaSota comprises the negative sense RNA transcribed from the cDNA sequence set forth in SEQ ID NO:13.

12. The method of any one of embodiments 1 to 11, wherein the APMV-4 is administered to the human subject intratumorally.

13. The method of any one of embodiments 1 to 12, wherein the APMV-4 is administered at a dose of 10⁶ to 10¹² pfu.

14. A recombinant APMV-4 comprising a packaged genome, wherein the packaged genome comprises a transgene comprising a nucleotide sequence encoding interleukin-12 (IL-12), interleukin-2 (IL-2), granulocyte-macrophage colony-stimulating factor (GM-CSF), interleukin-15 (IL-15) receptor alpha (IL-15Ra)-IL-15, human papillomavirus (HPV)-16 E6 protein or HPV-16 E7 protein, and wherein the APMV-4 has an intracerebral pathogenicity index in day-old chicks of the Gallus gallus species of less than 0.7.

15. The recombinant APMV-4 of embodiment 14, wherein the transgene is inserted between the AMPV-4 M and P transcription units of the packaged genome.

16. The recombinant APMV-4 of embodiment 14 or 15, wherein the transgene comprises a nucleotide sequence encoding IL-12.

17. The recombinant APMV-4 of embodiment 16, wherein the nucleotide sequence encoding IL-12 comprises the negative sense RNA transcribed from the nucleotide sequence of SEQ ID NO:16 or 17.

18. The recombinant APMV-4 of embodiment 16, wherein the packaged genome of the APMV-4 comprises the negative sense RNA transcribed from the cDNA sequence set forth in SEQ ID NO:14.

19. The recombinant APMV-4 of embodiment 14 or 15, wherein the transgene comprises a nucleotide sequence encoding IL-2.

20. The recombinant APMV-4 of embodiment 19, wherein the nucleotide sequence encoding IL-2 comprises the negative sense RNA transcribed from the nucleotide sequence of SEQ ID NO:15.

21. The recombinant APMV-4 of embodiment 14 or 15, wherein the transgene comprises a nucleotide sequence encoding IL-15Ra-IL15.

22. The recombinant APMV-4 of embodiment 21, wherein the nucleotide sequence encoding IL-15Ra-IL-15 comprises the negative sense RNA transcribed from the nucleotide sequence of SEQ ID NO:18.

23. The recombinant APMV-4 of embodiment 14 or 15, wherein the transgene comprises a nucleotide sequence encoding GM-CSF.

24. The recombinant APMV-4 of embodiment 23, wherein the nucleotide sequence encoding GM-CSF comprises the negative sense RNA transcribed from the nucleotide sequence of SEQ ID NO:21.

25. The recombinant APMV-4 of embodiment 14 or 15, wherein the transgene comprises a nucleotide sequence encoding HPV-16 E6 protein.

26. The recombinant APMV-4 of embodiment 25, wherein the nucleotide sequence encoding the HPV-16 E6 protein comprises the negative sense RNA transcribed from the nucleotide sequence of SEQ ID NO:19.

27. The recombinant APMV-4 of embodiment 14 or 15, wherein the transgene comprises a nucleotide sequence encoding HPV-16 E7 protein.

28. The recombinant APMV-4 of embodiment 27, wherein the nucleotide sequence encoding the HPV-16 E7 protein comprises the negative sense RNA transcribed from the nucleotide sequence of SEQ ID NO:20.

29. The recombinant APMV-4 of any one of embodiments 14 to 17 or 19 to 28, wherein the recombinant APMV-4 comprises an APMV-4 Duck/Hong Kong/D3/1975 strain backbone.

30. The recombinant APMV-4 of any one of embodiments 14 to 17 or 19 to 28, wherein the recombinant APMV-4 comprises an APMV-4 Duck/China/G302/2012 strain backbone, APMV4/mallard/Belgium/15129/07 strain backbone; APMV4Uriah-aalge/Russia/Tyuleniy_Island/115/2015 strain backbone, APMV4/Egyptian goose/South Africa/NJ468/2010 strain backbone, or APMV4/duck/Delaware/549227/2010 strain backbone.

31. A method for treating cancer, comprising administering to a human subject in need thereof a naturally occurring avian paramyxovirus serotype 8 (APMV-8), wherein the APMV-8 has an intracerebral pathogenicity index in day-old chicks of the Gallus gallus species of less than 0.7.

32. The method of embodiment 31, wherein the APMV-8 is APMV-8 Goose/Delaware/1053/1976.

33. The method of embodiment 31 or 32, wherein administration of the APMV-8 decreases tumor growth and increases survival in a BALBC syngeneic murine colon carcinoma tumor model as compared to tumor growth and survival in a BALBc syngeneic murine colon carcinoma tumor model administered phosphate buffered saline (PBS).

34. The method of embodiment 31 or 32, wherein administration of the APMV-8 results in a greater decrease in tumor growth and a longer survival time in a BALBc syngeneic murine colon carcinoma tumor model as compared to tumor growth and survival time in a BALBc syngeneic murine colon carcinoma tumor model administered a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A.

35. The method of embodiment 34, wherein the packaged genome of the modified NDV LaSota comprises the negative sense RNA transcribed from the cDNA sequence set forth in SEQ ID NO:13.

36. A recombinant APMV comprising a packaged genome, wherein the packaged genome comprises a transgene comprising a nucleotide sequence encoding interleukin-12 (IL-12), interleukin-2 (IL-2), granulocyte-macrophage colony-stimulating factor (GM-CSF), interleukin-15 (IL-15) receptor alpha (IL-15Ra)-IL-15, human papillomavirus (HPV)-16 E6 protein or HPV-16 E7 protein, and wherein the recombinant APMV has an intracerebral pathogenicity index in day-old chicks of the Gallus gallus species of less than 0.7, and the recombinant APMV comprises the APMV-6, APMV-7, APMV-8 or APMV-9 backbone.

37. The recombinant APMV of embodiment 36, wherein the recombinant APMV comprises the APMV-8 backbone.

38. The recombinant APMV of embodiment 37, wherein the recombinant APMV comprises the APMV-8 Goose/Delaware/1053/1976 backbone.

39. The recombinant APMV of embodiment 36, wherein the recombinant APMV comprises the APMV-7 backbone.

40. The recombinant APMV of embodiment 39, wherein the recombinant APMV comprises the APMV-7 Dove/Tennessee/4/1975 backbone.

41. The recombinant APMV of embodiment 36, wherein the recombinant APMV comprises the APMV-6 backbone.

42. The recombinant APMV of embodiment 41, wherein the APMV comprises the APMV-6 Duck/Hong Kong/199/1977 backbone.

43. The recombinant APMV of embodiment 36, wherein the recombinant APMV comprises the APMV-9 backbone.

44. The recombinant APMV of embodiment 43, wherein the recombinant APMV comprises the APMV-9 Duck/New York/22/1978 backbone.

45. The recombinant APMV of any one of embodiments 36 to 44, wherein the transgene is inserted between the AMPV M and P transcription units of the APMV packaged genome.

46. The recombinant APMV of any one of embodiments 36 to 45, wherein the transgene comprises a nucleotide sequence encoding IL-12.

47. The recombinant APMV of embodiment 46, wherein the nucleotide sequence encoding IL-12 comprises the negative sense RNA transcribed from the nucleotide sequence of SEQ ID NO:16 or 17.

48. The recombinant APMV of any one of embodiments 36 to 45, wherein the transgene comprises a nucleotide sequence encoding IL-2.

49. The recombinant APMV of embodiment 48, wherein the nucleotide sequence encoding IL-2 comprises the negative sense RNA transcribed from the nucleotide sequence of SEQ ID NO:15.

50. The recombinant APMV of any one of embodiments 36 to 45, wherein the transgene comprises a nucleotide sequence encoding IL-15Ra-IL15.

51. The recombinant APMV of embodiment 50, wherein the nucleotide sequence encoding IL-15Ra-IL-15 comprises the negative sense RNA transcribed from the nucleotide sequence of SEQ ID NO:18.

52. The recombinant APMV of any one of embodiments 36 to 45, wherein the transgene comprises a nucleotide sequence encoding GM-CSF.

53. The recombinant APMV of embodiment 52, wherein the nucleotide sequence encoding GM-CSF comprises the negative sense RNA transcribed from the nucleotide sequence of SEQ ID NO:21.

54. The recombinant APMV of any one of embodiments 36 to 45, wherein the transgene comprises a nucleotide sequence encoding HPV-16 E6 protein.

55. The recombinant APMV of embodiment 54, wherein the nucleotide sequence encoding the HPV-16 E6 protein comprises the negative sense RNA transcribed from the nucleotide sequence of SEQ ID NO:19.

56. The recombinant APMV of any one of embodiments 36 to 45, wherein the transgene comprises a nucleotide sequence encoding HPV-16 E7 protein.

57. The recombinant APMV of embodiment 56, wherein the nucleotide sequence encoding the HPV-16 E7 protein comprises the negative sense RNA transcribed from the nucleotide sequence of SEQ ID NO:20.

58. A method for treating cancer, comprising administering to a human subject in need thereof a recombinant APMV-4 of any one of embodiments 14 to 30.

59. The method of embodiment 58, wherein the recombinant APMV-4 is administered to the human subject intratumorally.

60. The method of embodiment 58 or 59, wherein the recombinant APMV-4 is administered at a dose of 10⁶ to 10¹² pfu.

61. A method for treating cancer, comprising administering to a human subject in need thereof a recombinant APMV of any one of embodiments 36 to 57.

62. The method of embodiment 61, wherein the recombinant APMV is administered to the human subject intratumorally.

63. The method of embodiment 61 or 62, wherein the recombinant APMV is administered at a dose of 10⁶ to 10¹² pfu.

64. The method of any one of embodiments 31 to 35, wherein the APMV-8 is administered to the human subject intratumorally.

65. The method of any one of embodiments 31 to 35, or 64, wherein the APMV-8 is administered at a dose of 10⁶ to 10¹² pfu.

66. A method of treating cancer, comprising administering a naturally occurring avian paramyxovirus serotype 6 (APMV-6) or 9 (APMV-9), wherein the APMV-6 or APMV-9 has an intracerebral pathogenicity index in day-old chicks of the Gallus gallus species of less than 0.7.

67. The method of embodiment 66, wherein the APMV-6 is APMV-6 Duck/Hong Kong/199/1977.

68. The method of embodiment 66, wherein the APMV-9 is APMV-9 Duck/New York/22/1978.

69. The method of embodiment 66, 67 or 68, wherein administration of the APMV-6 or APMV-9 decreases tumor growth and increases survival in a BALBC syngeneic murine colon carcinoma tumor model as compared to tumor growth and survival in a BALBc syngeneic murine colon carcinoma tumor model administered phosphate buffered saline (PBS).

70. The method of embodiment 66, 67 or 68, wherein administration of the APMV-6 or APMV-9 results in a greater decrease in tumor growth and a longer survival time in a BALBc syngeneic murine colon carcinoma tumor model as compared to tumor growth and survival time in a BALBc syngeneic murine colon carcinoma tumor model administered a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A.

71. The method of embodiment 70, wherein the packaged genome of the modified NDV LaSota comprises the negative sense RNA transcribed from the cDNA sequence set forth in SEQ ID NO:13.

72. The method of any one of embodiments 1 to 13, 31 to 35, or 58 to 71, wherein the cancer is melanoma, lung carcinoma, colon carcinoma, B-cell lymphoma, T-cell lymphoma, or breast cancer.

73. The method of any one of embodiments 1 to 13, 31 to 35, or 58 to 72, wherein the cancer is metastatic.

74. The method of any one of embodiments 1 to 13, 31 to 35, or 58 to 73, wherein the cancer is unresectable.

75. The method of any one of embodiments 1 to 13, 31 to 35, or 58 to 74 further comprising administering the subject a checkpoint inhibitor.

76. The method of any one of embodiments 1 to 13, 31 to 35, or 58 to 75 further comprising administering the subject a monoclonal antibody that specifically binds to PD-1 and blocks the binding of PD-1 to PD-L1 and PD-L2.

The invention is not to be limited in scope by the specific embodiments described herein. Indeed, various modifications of the invention in addition to those described will become apparent to those skilled in the art from the foregoing description and accompanying Figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

1.-76. (canceled)

77. A method of treating melanoma in a subject in need thereof, the method comprising administering to the subject a recombinant avian paramyxovirus serotype 4 (APMV-4) comprising a packaged genome, wherein the packaged genome comprises a transgene.

78. The method of claim 77, wherein the transgene comprises a nucleotide sequence encoding interleukin-12 (IL-12).

79. The method of claim 77, wherein the transgene comprises a nucleotide sequence encoding interleukin-2 (IL-2).

80. The method of claim 77, wherein the transgene comprises a nucleotide sequence encoding granulocyte-macrophage colony-stimulating factor (GM-CSF).

81. The method of claim 77, wherein the transgene comprises a nucleotide sequence encoding interleukin-15 (IL-15).

82. The method of claim 77, wherein the transgene comprises a nucleotide sequence encoding human papillomavirus (HPV)-16 E6 protein.

83. The method of claim 77, wherein the transgene comprises a nucleotide sequence encoding human papillomavirus (HPV)-16 E7 protein.

84. The method of claim 77, wherein the transgene is inserted between AMPV-4 M and P transcription units of the packaged genome.

85. The method of claim 77, wherein the recombinant APMV-4 comprises an APMV-4 Duck/Hong Kong/D3/1975 strain backbone.

86. The method of claim 77, wherein the recombinant APMV-4 comprises an APMV-4 Duck/China/G302/2012 strain backbone.

87. The method of claim 77, wherein the recombinant APMV-4 comprises an APMV4/mallard/Belgium/15129/07 strain backbone.

88. The method of claim 77, wherein the recombinant APMV-4 comprises an APMV4Uriah-aalge/Russia/Tyuleniy_Island/115/2015 strain backbone.

89. The method of claim 77, wherein the recombinant APMV-4 comprises an APMV4/Egyptian goose/South Africa/NJ468/2010 strain backbone.

90. The method of claim 77, wherein the recombinant APMV-4 comprises an APMV4/duck/Delaware/549227/2010 strain backbone.

91. The method of claim 77, wherein administration is intratumoral.

92. The method of claim 77, wherein administration is intravenous.

93. The method of claim 77, wherein the subject is human.

94. The method of claim 77, wherein the recombinant APMV-4 has an intracerebral pathogenicity index in day-old chicks of the Gallus gallus species of less than 0.7.

95. The method of claim 77, wherein administration of the recombinant APMV-4 decreases tumor growth and increases survival in a B16-F10 syngeneic murine melanoma model as compared to tumor growth and survival in B16-F10 syngeneic murine melanoma model administered phosphate buffered saline (PBS).

96. The method of claim 77, wherein administration of the recombinant APMV-4 results in a greater decrease in tumor growth and a longer survival time in a B16-F10 syngeneic murine melanoma model as compared to tumor growth and survival time in a B16-F10 syngeneic murine melanoma model administered a genetically modified Newcastle disease virus (NDV), wherein the genetically modified NDV is the NDV LaSota strain comprising a packaged genome, wherein the packaged genome comprises a nucleotide sequence encoding a mutated NDV LaSota F protein, wherein the mutated LaSota F protein has the mutation L289A.


SEQUENCE LISTING (52 sequences)

SEQ ID NO: 1 - DNA, 14904 bp, Avian paramyxovirus 2
accaaacaaggaataggtaagcaacgtaaatcttagataaaaccatagaatccgtgggggcgacatcgcctgaagccgatctcgagatcgataactccggttaattggtctcagcgtgaggagcttatctgtctgtggcaatgtcttctgtgttttcagaataccaggctcttcaggaccaactggtcaagcctgccactcgaagggctgatgtggcatcgactggattgttgagagcggagataccagtttgtgtaaccttgtctcaggacccaactgatagatggaacctcgcatgtctcaatctgcgatggctgataagtgagtcctctactactcccatgagacaaggggcgatcctgtcactgctgagcttgcactctgacaacatgcgagctcacgcaacccttgcagcgagatccgctgatgctgccatcactgtgcttgaggttgacgccatagacatggcggatggcacaatcacttttaatgccagaagtggagtatccgagaggcgcagcacacagctcatggcaatcgcaaaagatctgccccgctcttgttccaatgactcaccattcaaagatgacactatcgaggatcgcgacccccttgacctgtccgagactatcgatagactgcaggggattgctgcccaaatctggatagcggccatcaagagcatgactgccccggatactgctgcggagtcagaaggcaagaggcttgcaaagtaccaacaacaaggccgcttggtgcgacaggtgttagtgcatgatgcggtgcgtgcggaattcctacgtgtcatcagaggcagcctggtcttacggcaattcatggtatcagaatgtaagagggcagcatccatgggtagcgagacatctaggtactatgccatggtgggtgacatcagcctctacatcaagaatgcaggacttaccgccttcttcttgacactcagatttggtattgggacacactaccccactcttgccatgagtgtgttctctggagaactgaagaagatgtcgtccttgatcaggctgtataagtcaaaaggggaaaatgctgcatacatggcattcctggaggatgcggacatgggaaactttgcgcctgctaactttagtactctctactcctatgcaatgggggtaggtacagtgctggaagcatcagttgcgaaataccagttcgctcgagagttcaccagtgagacatacttcaggcttggggttgagaccgcacagaaccaacagtgcgctctagatgaaaagaccgccaaggagatggggcttactgatgaagccagaaagcaggtgcaagcattggctagcaacatcgagcaggggcaacattcaatgcccatgcaacaacagcccacattcatgagtcagccctaccaggatgacgatcgtgaccagccaagcaccagcagaccagagccaagaccatcgcaattgacaagccaatcagcagcacaggacaatgatgcggcctcattagattggtgaccgcaatcagctcagccaagccattgttggacgcaggacattcaaatcatacattgccctaagagtattaaagtgatttaagaaaaaaggaccctgggggcgaagttgtcccaatccaggcaggcgctgaaaccgaatccctccaacctccgagccccaggcgaccatggagttcaccgatgatgccgaaattgctgagctgttggacctcgggacctcagtgatccaagagctgcagcgagccgaagtcaagggcccgcaaacaaccggaaagcccaaagttcccccggggaacactaagagcctggctactctctgggagcatgagactagcacccaagggagtgcattgggcacacccgagaacaacacccaggcacccgatgacaacaacgcaggtgcagatacgccagcgactaccgacgtccatcgcactctggataccatagacaccgacacaccaccggaagggagcaagcccagctccactaactcccaacccggtgatgaccttgacaaggctctttcgaagctagaggcgcgcgccaagctcggaccagatagggccagacaggttaaaaaggggaaggagatcgggtcgagcacagggacgagggaggcagccagtcaccacatggaagggagccgacagtcggagccaggagcgggcagccgagcacagccacaaggccatggcgaccgggacacaggagggagtactcattcatctctcgagatgggagactggaagtcacaagctggtgcaacccagtctgctctcccattagaagcgagcccaggagagaaaagtgcacatgtggaacttgcccagaatcctgcattttatgcaggcaacccaactgatgcaattatggggttgacaaagaaagtcaatgatctagagacaaaattggctgaggtattgcgtctgttaggaatactccccggaataaagaatgagattagtcagctgaaagcaaccgtggctctgatgtcaaatcagattgcctccattcagattcttgatcctgggaatgccggagtcaaatcccttaatgagatgaaagccctgtcaaaagcagccagcatagttgtggcaggtccaggagtccttcctcctgaggtcacagaaggaggactgatcgcgaaagatgagctagcaaggcccatccccatccaaccgcaacgagactccaaacccaaagacgacccgcacacatcaccaaatgatgtccttgctgtacgcgctatgatcgacacccttgtggatgatgagaagaagagaaagagattaaaccaggcccttgacaaggcaaagaccaaggatgacgtcttaagggtcaagcggcagatatacaatgcctaggagtccatttgtctaaagaacctccaatcatatcaccagtttcgtgccacatgcttccctgccgagaatctagccgacacaaaaactaaatcatagtttaacaaaaaagaagtttgggggcgaagtctcacatcatagagcacccttgcattctaaaatggctcaaacaaccgtcaggctgtatatcgatgaagctagtcccgacattgaactgttgtcttacccactgataatgaaagacacaggacatgggaccaaagagttgcagcagcaaatcagagttgcagagatcggtgcattgcagggagggaagaatgaatcagttttcatcaatgcatatggctttgttcagcaatgcaaagttaaaccgggggcaacccaattcttccaggtagatgcagctacaaagccagaagtggtcactgcagggatgattataatcggtgcagtcaagggggtggcaggcatcactaagctggcagaagaggtgttcgagctggacatctccatcaagaagtccgcatcattccatgagaaggttgcggtgtcctttaatactgtgccactatcactcatgaattcgaccgcatgcagaaatctgggttatgtcacaaacgctgaggaggcgatcaaatgcccgagcaaaatacaagcgggtgtgacgtacaaatttaagataatgtttgtctccttgacacgactgcataacgggaaattgtaccgtgtccccaaggcagtgtatgctgtagaggcatcagctctatataaagtgcaactggaagtcgggttcaagcttgacgtggccaaggatcacccacacgttaagatgttgaagaaagtggaacggaatggtgagactctgtatcttggttatgcatggttccacctgtgcaacttcaagaagacaaatgccaagggtgagtcccggacaatctccaacctagaagggaaagtcagagctatggggatcaaggtttccttgtacgacttatgggggcctactttggtggtgcaaatcacaggtaagaccagcaagtatgcacaaggtttcttttcaaccacaggtacctgctgcctcccagtgtcgaaggctgcccctgagctggccaaacttatgtggtcctgcaatgcaacaatcgttgaagctgcagtgattatccaagggagtgataggagggcagtcgtgacctcagaggacttggaagtatacggggcagttgcaaaagagaagcaggctgcaaaaggatttcacccgttccgcaagtgacacgtggggccgcacacctcattaccccagaagcccgggcaactgcaaattcacgcttatataatccaattaccatgatctagaactgcaatcgatactaatcgctcattgatcgtattaagaaaaaacttaactacataacttcaacattgggggcgacagctccagactaagtgggtggctaagctctgactgataaggaatcatgaatcaagcactcgtgattttgttggtatctttccagctcggcgttgccttagataactcagtgttggctccaataggagtagctagcgcacaggagtggcaactggcggcatatacaacgaccctcacagggaccatcgcagtgagatttatcccggtcctgcctgggaacctatcaacatgtgcacaggagacgctgcaggaatataatagaactgtgactaatatcttaggcccgttgagagagaacttggatgctctcctatctgacttcgataaacctgcatcgaggttcgtgggcgccatcattgggtcggtggccttgggggtagcaacagctgcacaaatcacagccgccgtggctctcaatcaagcacaagagaatgcccggaatatatggcgtctcaaggaatcgataaagaaaaccaatgcggctgtgttggaattgaaggatggacttgcaacgactgctatagctttggacaaagtgcaaaagtttatcaatgatgatattataccacagattaaggacattgactgccaggtagttgcaaataaattaggcgtctacctctccttatacttaacagagcttacaactgtatttggttctcagatcactaatcctgcattatcaacgctctcttaccaggcgctgtacagcttatgtggaggggatatgggaaagctaactgagctgatcggtgtcaatgcaaaggatgtgggatccctctacgaggctaacctcataaccggccaaatcgttggatatgaccctgaactacagataatcctcatacaagtatcttacccaagtgtgtctgaagtgacaggagtccgggctactgagttagtcactgtcagtgtcactacaccaaaaggagaagggcaggcaattgttccgagatatgtggcacagagtagagtgctgacagaggagttggatgtctcgacttgtaggtttagcaaaacaactctttattgtaggtcgattctcacacggcccctaccaactttgatcgccagctgcctgtcagggaagtacgacgattgtcagtacacaacagagataggagcgctatcttcgagattcatcacagtcaatggtggagtccttgcaaactgcagagcaattgtgtgtaagtgtgtctcacccccgcatataataccacaaaacgacattggctccgtaacagttattgactcaagtatatgcaaggaagttgtcttagagagtgtgcagcttaggttagaaggaaagctgtcatcccaatacttctccaacgtgacaattgacctttcccaaatcacaacgtcagggtcgctggatataagcagtgaaattggtagcattaacaacacagttaatcgggtcgacgagttaatcaaggaatccaacgagtggctgaacgctgtgaacccccgccttgtgaacaatacgagcatcatagtcctctgtgtccttgccgccctgattattgtctggctaatagcgctgacagtatgcttctgttactccgcaagatactcagctaagtcaaaacagatgaggggcgctatgacagggatcgataatccatatgtaatacagagtgcaactaagatgtagagaggttgaataagcctaaacatgatatgatttaagaaaaaattggaaggtgggggcgacagcccattcaatgaagggtgtacactccaacttgatcttgtgacttgatcatcatactcgaggcaccatggatttcccatctagggagaacctggcagcaggtgacatatcggggcggaagacttggagattactgttccggatcctcacattgagcataggtgtggtctgtcttgccatcaatattgccacaattgcaaaattggatcacctggataacatggcttcgaacacatggacaacaactgaggctgaccgtgtgatatctagcatcacgactccgctcaaagtccctgtcaaccagattaatgacatgtttcggattgtagcgcttgacctacctctgcagatgacatcattacagaaagaaataacatcccaagtcgggttcttggctgaaagtatcaacaatgttttatccaagaatggatctgcaggcctggttcttgttaatgaccctgaatatgcaggggggatcgctgtcagcttgtaccaaggagatgcatctgcaggcctaaatttccagcccatttctttaatagaacatccaagttttgtccctggtcctactactgctaagggctgtataaggatcccgaccttccatatgggcccttcacattggtgttactcacataacatcattgcatcaggttgccaggatgcgagccactccagtatgtatatctctctgggggtgctgaaagcatcgcagaccgggtcgcctatcttcttgacaacggccagccatctcgtggatgacaacatcaaccggaagtcatgcagcatcgtagcctcaaaatacggttgtgatatcctatgcagtattgtgattgaaacagagaatgaggattataggtctgatccggctactagcatgattataggtaggctgttcttcaacgggtcatacacagagagcaagattaacacagggtccatcttcagtctattctctgctaactaccctgcggtggggtcgggtattgtagtcggggatgaagccgcattcccaatatatggtggggtcaagcagaacacatggttgttcaaccagctcaaggattttggttacttcacccataatgatgtgtacaagtgcaatcggactgatatacagcaaactatcctggatgcatacaggccacctaaaatctcaggaaggttatgggtacaaggcatcctattgtgcccagtttcactgagacctgatcctggctgtcgcttaaaggtgttcaataccagcaatgtgatgatgggggcagaagcgaggttgatccaagtaggctcaaccgtgtatctataccaacgctcatcctcatggtgggtggtaggactgacttacaaattagatgtgtcagaaataacttcacagacaggtaacacactcaaccatgtagaccccattgcccatacaaagttcccaagaccatctttcaggcgagatgcgtgtgcgaggccaaacatatgccctgctgtctgtgtctccggagtttatcaggacatttggccgatcagtacagccaccaataacagcaacattgtgtgggttggacagtacttagaagcattctattccaggaaagacccaagaatagggatagcaacccagtatgagtggaaagtcaccaaccagctgttcaattcgaatactgagggagggtactcaaccacaacatgcttccggaacaccaaacgggacaaggcatattgtgtagtgatatcagagtacgctgatggggtgttcggatcatacaggatcgttcctcagcttatagagattagaacaaccaccggtaaatctgagtgatgcatcaatcctaaattggaatgaccaatcaaaagctacgtagtgtctaacagcattgcgaagcctggtttaagaaaaaacttgggggcgaatgcccatcaaccatggatcaaactcaagctgacactataatacaacctgaagtccatctgaattcaccacttgttcgcgcaaaattggttcttctatggaaattgactgggttacctttgccgtctgatttgagatcatttgtactaactacacatgcagctgatgaccaaatcgcaaaaaatgagactaggatcaaggccaaaattaattccctaatcgataacttaatcaaacactgcaaggcaaggcaagtggcactttcagggttgacacctgtcgtacatccaacaactctacagtggttgctatccatcacatgtgaacgagcagaccaccttgcaaaagtacgcgagaaatcagttaagcaagcaatgtcagagaagcaacacgggtttagacatctcttttcggcagtaagtcatcagttagttggaaacgccacactgttctgtgcacaagactctagcaccgtgaatgtcgactctccttgctcatcaggttgtgagaggctgataatagactctattggagccttacaaacacgatggacaagatgtaggtgggcttggcttcacattaaacaggtaatgagataccaggtgcttcagagtcgcctacacgctcatgccaattctgttagcacatggtctgaggcgtgggggttcattgggatcacaccagatatagtccttattgtagactataagagcaaaatgtttactatcctgaccttcgaaatgatgctgatgtattcagatgtcatagagggtcgtgataatgtggtagctgtaggaagtatgtcaccaaacctacagcctgtggtggagaggattgaggtgctgtttgatgtagtggacaccttggcgaggaggattcatgatcctatttatgatctggttgctgccttagaaagcatggcatacgctgccgtccaattgcacgatgctagtgagacacacgcaggggaattcttttcgttcaatttgacagaaatagagtccactcttgcccccttgctggatcctggccaagtcctatcggtgatgaggactatcagttattgttacagtgggctatcgcctgaccaagctgcagagttgctctgtgtgatgcgcttatttggacaccctctgctctccgcacaacaagcagccaaaaaagtccgggagtctatgtgtgcccctaaactgttagagcatgatgcaatactgcaaactctatctttcttcaagggaatcataatcaatggctacaggaaaagtcattctggagtatggcctgcaattgacccagattctatagtggacgatgaccttagacagctgtattacgagtcggcagaaatttcacatgctttcatgcttaagaaatatcggtaccttagtatgattgagttccgcaagagcatagagtttgacttaaatgatgacctgagcacattccttaaagacaaagcaatctgcaggccaaaagatcaatgggcacgcatcttccggaaatcattgttcccttgcaaaacgaaccttggcactagtatagatgttaaaagtaatcgactgttgatagattttttggagtcacatgacttcaatcctgaggaagaaatgaagtatgtgactacgctagcatacctggcagataatcaattctcagcatcatattcactgaaggagaaagagatcaagactactggccggatcttcgccaaaatgaccaggaaaatgaggagctgtcaagtaatattggaatcactattgtccagtcacgtctgcaaattctttaaggagaacggtgtgtcaatggaacaactgtctttgacaaagagcttgcttgcaatgtcacagttagcacccaggatatcttcagttcgccaggcgacagcacgtagacaggacccaggactcagccactctaatggttgtaatcacattgtaggagacttaggcccacaccagcaggacagaccggcccggaagagtgtagtcgcaaccttccttacaacagatcttcaaaaatattgcttgaattggcgatatgggagtatcaagcttttcgcccaagccttaaaccagctattcggaatcgagcatgggtttgaatggatacacctgagactgatgaatagcaccctgtttgtcggggacccattctcgcctcctgaaagcaaagtgctgagtgatcttgatgatgcgcccaattcagacatatttatcgtgtccgccagaggggggattgaagggttatgccagaagctgtggaccatgatttcaataagcataatccattgcgtggctgagaagataggagcaagggttgcggcgatggttcagggagataatcaggtaattgcaatcacgagagagctgtataagggagagacttacacgcagattcagccggagttagatcgattaggcaatgcattttttgctgaattcaaaagacacaactatgcaatgggacataatctgaagcccaaagagacaatccaaagtcaatcattctttgtgtattcgaaacggattttctgggaagggagaattcttagtcaagcactgaagaatgctaccaaactatgcttcattgcagatcacctcggggataatactgtctcatcatgcagcaatctagcctctacgataacccgcttggttgagaatgggtatgaaaaggacacagcattcattctgaatatcatctcagcaatgactcagttgctgattgatgagcaatattccctacaaggagactactcagctgtgagaaaactgattgggtcatcaaattaccgtaatctcttagtggcgtcgctcatgcctggtcaggttggcggctataatttcttgaatatcagtcgcctattcacacgcaatattggtgatccagtaacatgcgccatagcagatctgaagtggttcattaggagcgggttaatcccagagttcatcctgaagaatatattactacgagatcccggagacgatatgtggagtactctatgtgctgacccttacgcattaaatatcccctacactcagctacccacaacatacctgaagaagcatactcagagggcattactatccgattctaataatccgcttcttgcaggggtgcaattggacaatcaatacattgaagaggaggagtttgcacgattccttttggatcgggaatccgtgatgcctcgagtggcacacacaatcatggagtcaagtatactagggaagagaaagaacatccagggtttaatcgacactacccctacaatcattaagactgcactcatgaggcagcccatatctcgtagaaagtgtgataaaatagttaattactcgattaactacctgactgagtgccacgattcattattgtcctgtaggacattcgagccaaggaaggaaataatatgggagtcagctatgatctcagtagaaacttgcagtgtcacaattgcggagttcctgcgcgccaccagctggtccaacatcctgaacggtaggactatttcgggtgtaacatctccagacactatagagctgctcaaggggtcattaattggagagaatgcccattgtattctttgtgagcagggagacgagacattcacgtggatgcacttagccgggcccatctatataccagacccgggggtgaccgcatccaagatgagagtgccgtatcttgggtcaaagacagaggaaaggcgtacggcatccatggccaccattaagggcatgtctcaccacctaaaggccgctttgcgaggagcctctgtgatggtgtgggcctttggtgatactgaagaaagttgggaacatgcctgccttgtggccaatacaaggtgcaagattaatcttccgcagctacgcctgctgaccccgacaccaagcagctctaacatccaacatcgactaaatgatggtatcagcgtgcaaaaatttacacctgctagcttatcccgagtggcgtcatttgttcacatttgcaacgatttccaaaagctagagagagatggatcttccgtagactctaacttgatatatcagcaaatcatgctgactggtctaagtattatggagacacttcatcctatgcacgtctcatgggtatacaacaatcagacaattcacttacataccggaacatcgtgttgtcctagggaaatagagacaagcattgttaatcccgctaggggagaattcccaacaataactctcacaactaacaatcagtttctgtttgattgtaatcccatacatgatgaggcacttacaaaactgtcagtaagtgagttcaagttccaggagcttaatatagactcaatgcagggttacagtgctgtgaacctgctgagcagatgtgtggctaagctgataggggaatgcattctggaagacggtatcggatcgtcaatcaagaatgaagcaatgatatcatttgataactctatcaactggatttctgaagcactcaatagtgacctgcgtttggtattcctccagctggggcaagaactactttgtgacctggcgtaccaaatgtactatctgagggtcatcggctatcattccatcgtggcatatctgcagaatactctagaaagaattcctgttatccaactcgcaaacatggcactcaccatatcccacccagaagtatggaggagagtgacagtgagcggattcaaccaaggttaccggagtccctatctggccactgtcgactttatcgccgcatgtcgtgatatcattgtgcaaggtgcccagcattatatggctgatttgttgtcaggagtagagtgccaatatacattctttaatgttcaagacggcgatctgacaccgaagatggaacaatttttagcccggcgcatgtgcttgtttgtattgttaactgggacgatccgaccactcccaatcatacgatcccttaatgcgattgagaaatgtgcaattctcactcagttcttgtattacctaccgtcagtcgacatggcagtagcagacaaggctcgtgtgttatatcaactgtcaataaatccgaaaatagatgctttagtctccaacctttatttcaccacaaggaggttgctttcaaatatcaggggagattcttcttcacgagcgcaaattgcattcctctacgaggaggaagtaatcgttgatgtgcctgcatctaatcaatttgatcagtaccatcgtgaccccatcctaagaggaggtctatttttctctctctccttaaaaatggaaaggatgtctctgaaccgatttgcagtacagaccctgccaacccaggggtctaactcgcagggttcacgacagaccttgtggcgtgcctcaccgttagcacactgccttaaatcagtagggcaggtaagtaccagctggtacaagtatgctgtagtgggggcgtctgtagagaaagtccaaccaacaagatcaacaagcctctacatcggggagggcagtgggagtgtcatgacattattagagtatctggaccctgctacaattatcttctacaactcgctattcagcaatagcatgaaccctccacaaaggaatttcggactgatgcccacacagtttcaggactcagtcgtgtataaaaacatatcagcaggagttgactgcaagtacgggtttaagcaagtctttcaaccattatggcgtgatgtagatcaagaaacaaatgtggtagagacggcgttcctaaactatgtgatggaagtagtgccagtccactcttcgaagcgtgtcgtatgtgaagttgagtttgacagggggatgcctgacgagatagtaataacagggtacatacacgtgctgatggtgaccgcatacagtctgcatcgaggagggcgtctaataatcaaggtctatcgtcactccgaggctgtattccaattcgtactctctgcgatagtcatgatgtttggggggcttgatatacaccggaactcgtacatgtcaactaacaaagaggagtacatcatcatagctgcggcgccggaggcattaaactattcctctgtaccagcaatattgcagagggtgaagtctgttattgaccagcagcttacattaatctctcctatagatctagaaagattgcgccatgagactgagtctctccgtgagaaggagaataatctagtaatatctctgacgagagggaagtatcaactccggccgacacagactgatatgcttctatcatacctaggtgggagattcatcaccctattcggacagtctgctagggatttgatggccactgatgttgctgaccttgatgctaggaagattgcattagttgatctactgatggtggaatccaacattattttaagtgagagcacagacttggaccttgcactgttgctgagcccgtttaacttagacaaagggcggaagatagttaccctagcaaaggctactacccgccaattgctgcccgtgtatatcgcatcagagataatgtgcaatcggcaggcattcacacacctgacatcaattatacagcgtggtgtcataagaatagaaaacatgcttgctacaacggaatttgtccgacagtcagttcgcccccagttcataaaggaggtgataactatagcccaagtcaaccaccttttttcagatctatccaaactcgtgctttctcgatctgaagtcaagcaagcacttaaatttgtcggttgctgtatgaagttcagaaatgcaagcaattaaacaggattgttattgtcaaatcaccggttactatagtcaaattaatatgtaaagttccctctttcaagagtgattaagaaaaaacgcgtcaaaggtggcggtttcactgatttgctcttggaagttgggcatcctccagccaatatatcggtgccgaaatcgaaagtctgacagctgatttggaatataagcactgcataatcactgagttacgttgctttgctattccatgtctggt

SEQ ID NO: 2 - DNA, 16182 bp, Avian paramyxovirus 3
actaaacagaaagttaataagtgtttgtaacgtccgattaagtagccagattaataggagcggaagtcctaaattccgcgtccgactgcgaatttcaataactatggcaggtatcttcaatacatatgagttgttcgtcaaggaccaaacatgcatgcacaagcgggcagcaagtctcatatcaggggggcagctcaaaagcaacatcccagtattcattaccaccagggatgacccggccgtgaggtggaatcttgtttgctttaatctaaggttaattgtcagtgagtcctcaacatcagttattcgccaaggagcaatgatctcacttttgtcagtcacagcaagtaacatgagggctttagcagcaatcgctggtcagacagatgagtcaatgattaatataattgaagttgttgatttcaatgggttagagccacaatgtgatccaaggagtggccttgatgctcagaagcaagacatgtttaaagacattgcaagtgatatgccgaaggttctcggaagtggcacacctttccagaatgtaagtgcagagaccaacaatccagaggatacacacatgttcttacgctcagcaatcagcgtcctgactcaaatctggattttggtagcaaaagccatgactaatatcgaaggtagtcatgaggccagtgatagaaggcttgcgaaatacacccagcagaacagaattgaccggcgctttatgctggcccaagccactcggactgcatgccagcaaataataaaggactcactaacaattagaaggtttctggtcacggaacttcggaagtcgcgaggggctcttcatagtgggtcatcatattatgcaatggtaggagatatgcaagcatacatctttaatgctggacttactcctttcctcacaacactcaggtatggtattggtaccaaataccacgctctcgcaatcagttctctgacgggagaccttaataagattaagggattgctaacactgtacaaggaaaaggggagtgacgcagggtatatggcattattagaggatgcagattgcatgcaatttgcaccagggaactatgcgttgctgtactcgtatgcaatgggagttgccagtgtccatgatgaaggcatgagaaactaccagtatgcaaggcggtttctgcacaaaggcatgtaccagtttggaagagacattgcaacacaacaccagcatgcattggatgagtctcttgctcaggaaatgagaatcaccgaggcggaccgggccaatctcaaagtaatgatggcaaatatcggtgaggcttcccattacagtgatattcccagtgcgggccccagtggcataccagcatttaacgatccaccagaagagttatttggagagccctcatacaggaagttgcccgaagagcctcaagttgtagaactacaagaccgggatgacgatgagcaagatgaatatgatatgtaatccttcaggagaacacccccaccacccaacagcccccgaaaattaaaaacactccctccccgacaacccgcacaccccacggccatcacccccccatcagcacccaatcccaagcgcagacaggccaccgcctccacccagaaccccaggacccaaatccccactatatctttaagaaaaaaagacctgatgtgtacgaggagaaaaataattgatgacaagcggagaaaataggagcggaagtatccctcctaacaagatagacacaattatcatggatcttgaattcagcagtgaggaggcagttgcagctttgctcgacgtgagttcatccactatcacagagttcctaagcaaacaaagcatccccgatccgggattcctaaattcaccttcccagtcaagcagtccctcccctgaaccaagcacctctactaccggtgacttcctctcacagctatcaggtgatatccctgataccaccacatcaggtgtagaaccatcagcacctctagatacaggtgacacctcgttggtacaacatattgaggagggactgccctcagacttctacatacccaaagtcaacaactatcattcgaacctttttaaagggggctcctccctgctcgcaacggcggaatcccctggtctgacagtgacccacaaagatacgactacaccggagtccacaccggttatggcgaagaagaagaagaagcagaagcactgcaaagtgcccgcatcttcggcgtaccaacacatagacaatctgggcaccggagagagtactccattgcatgggatgcaagatcaggaaccttccaaaccgaaacatggtgtaaccccgcatgttccccagtcacagccctcccaaagcagtatagatgtgcttgccgacaatgtcccaaattctgtgacctctgtttcaatcccgctgactatggtggaatcattgatctcgcaagtgtcaaagttatcggaccaagtctctcagatccagaaattggtgagcacacttccccaaattaagaccgacatagcatcaatcaggaacatgcaggcggccctagaaggtcaaattagtatgataaggatactcgaccccggcaacaacacagagtcatccctaaataccctccgcaactctggaaatcgggctccagtagtgatttgcggaccgggcgaccctcaccgcagtctgatcaaaagcgagaacccgactatctgcctggatgaactagctcggccaactcaagccaacagtcctccaaaatctcaagataaccaaagggatctatccgctcaacgacacgcaatcacagctctgctagaaacccgcgttgcacccggacctaagagagatcgcctgatggaaatggtagtagcagcgaaatcagcaagtgatctcatcaaagtcaagagaatggcaattcttggtcaataaaccgactcagcaccacattgtctgtgactctacacttgtgcggcaaaccaacattgacctccaaacacttttctgcagtacgcaaggcttaacacaatcagcagcatgcatatcgagcggcccaccctcacaacccatctagctctcttattttatctattgctttataaaaaaccaaaatgattataactaaacaatctcaacaatttgcaatgataacaacaccatacgatcactaggggcggaagcccaaaataacccaaggaccaatctccgagtccaggccagacacaggcaacccatcagcacagagccaagcaaccaaaatggcagcacaccccaaccatgccaacccatcctcgtcaatcagcctcatgcatgatgatccatccatccagacgcaacttcttgcctttccgctgatcagtgaaaagaccgagacgggcactaccaaacttcaacctcaagtcagaatgcagtcatttctctcaactgacagccaaaagtaccacctggtattcataaatacgtatggtttcatagccgaggacttcaactgtagtcctaccaatggattcgttcctgcgttgtttcaaccgaaatctaaggtattgtcttcagcaatggttacccttggtgcagttcctgcagatacagtcctgcaggacttacaaaaagaccttatagccatgcgatttaaggtcaggaagagtgcatctgctaaagaactcatactattctctactgataatattccagcaacacttacaggatcatctgtttggaaaaacaggggtgttattgcagacaccgccacatccgtgaaggcccccggcagaatctcctgtgatgcagtctgcagttattgcattactttcatatcattctgtttcttccactcatctgccttattcaaggtgcccaagccactgcttaattttgagacagccgttgcctattctctagtcctgcaggttgaattggaattcccgaacataaaggacaccctacatgagaaatatttaaagaacaaggactctaaatggtactgtaccattgacatacacatagggaacctcctgaaaaggactgcaaaacagagaaggcgtacaccatctgaaatcactcaaaaggtgcgcagaatgggctttcggattggactctacgatctttggggccctacaatagtggtcgaattaactggctcatcgagcaaatcgctccagggattcttctccagtgagagactggcttgccatcctatttcacaatacaacccacatgtcggtcaactgatttgggcacatgatgtttcaataacaggctgtcatatgataatatctgaacttgagaaaaagaaagctttggccatggctgacctcactgtaagtgatgcagttgctatcaatactacaataaaggagttggttcctttccgcttgttcaggaaataaatcactcactgccgccagcttaccactagtaacaaattacaaccatcacctataacctaacaaaccaaatgcatgcacctaaccttctgggttgaatgagaagcttggattatattcatgattagctaacacgaatttattgcttaaattgcttataccggtaataactcaaatattccactaaccaaatttaattaaaaatattaataatcattagcaacatccgatcggaatcttcaggggcggaaggaccaccgccacaacaccccaccacaccagacctccccgcgcccccacaagaccggccacaccaaacaaaaagcccccccaaccccccacaccctccccgacagcccgacaaaaaacccccccaaaaaacagatcgcccacacacagatcagaatggcctccccaatggtcccactactcatcataacggtagtacccgcactcatttcaagtcaatcagctaatattgataagctcattcaagcagggattatcatgggctcagggaaggaactccacatttatcaagaatctggctctcttgatttgtatcttagactattgccagttatcccttcaaatctttctcattgccagagtgaagtaataacacaatataactcgactgtaacgagactattatcaccaattgcaaaaaatctaaaccatttgctacaaccgagaccgtctggcaggttatttggcgctgtaattggatcgattgccttaggggtagctacatccgcacagatttcagctgctatagcattggtccgtgctcaacagaatgcaaacgatatcctcgctcttaaagctgcaatacaatctagtaatgaggcaataaaacaacttacttatggccaagaaaagcaactactagcaatatcaaaaatacaaaaagccgtaaatgaacaagtaatccctgcattgactgcacttgactgtgcagttcttggaaataaactagctgcacaactgaacctctacctcattgaaatgacgactatttttggtgaccaaataaataacccagtcctaactccaataccactcagttatctcctgcggttgacaggctctgagttaaatgatgtattattacaacagactcgatcctctttgagcctaatccaccttgtctctaaaggcttattaagtggtcagattataggatatgacccttcagtacaaggcatcattatcagaataggactgatcaggactcaaagaatagatcggtcactagttttccwaccttacgtattaccaattactattagttctaacatagccacaccaattatacccgactgtgtggtcaagaagggagtaataattgagggaatgcttaagagtaattgtatagaattggaacgagatataatttgcaagactatcaacacataccaaataactaaggaaactagagcatgcttacaaggtaatataacaatgtgtaagtaccagcagtccaggacacagttgagcaccccctttattacatataatggagttgtaattgcaaattgtgatttggtatcatgccgatgcataagaccccctatgattatcacacaagtaaaaggttaccctctgacaattataaataggaatttatgtaccgagttgtcggtggataatttaattttaaatattgaaacaaaccataacttttcattaaaccctactattatagattcacaatcccggcttatagctactagtccattagaaatagatgcccttattcaagatgcgcaacatcacgcggctgcggcccttcttaaagtagaagaaagcaatgctcacttattaagagttacagggctgggctcatcaagttggcacatcatacttatattaacattgcttgtatgcaccatagcatggctcattggtttatctatttatgtctgccgcattaaaaatgatgactcgaccgacaaagaacctacaacccaatcatcgaaccgcggcattggggttggatctatacaatatatgacataatgagccgcctgtatatcaagcccaagtatcgacccctcccaccatcctcgaccgccgccactagcagcacaggaagtaatcagttacagtggcatcagcagtcccatgttgagacacaccagtacaccctagtttctagtaaaacccccagttctattttctgcattccattaatttataaaaaaatgccatgatactcgtgcgagtgtaacatagtaactaggggcggaagcctaccgccaaatcagcacacacccccccaacatggagccgacaggatcaaaagttgacattgtcccttcccaaggtaccaagagaacatgtcgaaccttttatcgcctcttaattcttattttgaatcttattataattatattaacaattatcagtatttatgtctctatctcaacagatcaacacaaattgtgcaataatgaggctgactcacttttacactcaatagtagaacccataacagtccccctaggaacagactcggatgttgaggatgaattacgtgagattcgacgtgatacaggcataaatattcctatccaaattgacaacacagagaacatcatattaactacattagcaagtatcaactctaacattgcacgccttcataacgccaccgatgaaagcccaacatgcctgtcaccagttaatgatcccaggtttatagcagggattaataagataaccaaagggtcgatgatatataggaatttcagcaatttgatagaacatgttaactttataccatctccaacgacattatcaggctgtacaagaattccatctttttcactatctaaaacacattggtgttactcgcataatgtaatatctactggttgtcaagaccatgctgcgagttcacagtatatttccataggaatagtagatacaggattgaataatgagccctatttgcgtacaatgtcttcacgcttgctaaatgatggcctaaatagaaagagctgctctgtcacagccggcgctggtgtctgttggctattgtgtagtgttgtaacagaaagtgaatcagctgactacagatcaagagcccccactgcaatgattctcggaaggttcaatttttatggtgattacactgaatcccctgttcctgcatctttgttcagcggtcgtttcactgctaattaccctggagttggctcaggaacccaattaaatgggaccctttattttccaatatatgggggtgttgttaacgactctgatattgagttatcgaaccgagggaagtcattcagacctaggaaccctacaaacccatgtccagatcctgaggtgacccaaagtcagagggctcaggcaagttactatccgacaaggtttggcaggctgctcatacaacaagcaatactagcttgtcgtattagtgacactacatgcactgattattatcttctatactttgataataatcaagtcatgatgggtgcagaagcccgaatttattatttaaacaatcagatgtacttatatcaaagatcttcgagttggtggccgcatccgcttttttacagattctcactgcctcattgtgaacctatgtctgtctgtatgatcaccgatacacacttaatattgacatatgctacctcacgccctggcacttcaatttgtacaggggcctcgcgatgtcctaataactgtgttgatggtgtctatacagacgtttggcccttgactgagggtacaacacaagatccagattcctactacacagtattcctcaacagtcccaaccgcaggatcagtcctacaattagcatttacagctacaaccagaagattagctctcgtctggctgtaggaagtgaaataggagctgcttacacgaccagtacatgttttagcaggacagacactggggcactatactgcatcactataatagaagctgtaaacacaatctttggacaataccgaatagtaccgatccttgttcaactaattagtgactaggaaatgatgtttaattactcgatgttgagtaaatgatcctagaacttctccttagaatgatatacatcgcttgtactataatcaagtaacgggcagcgggtgatccatattaaataatatatgcattaagcagatacaaatcttcactttgtcaatcagaattgattattgcacctttgccacgtagataactaagcatttaagaaaaaacttcactatcactctttgagtcgctgaagtgagatttcagaaaggtatgcatctaagaagtaggagcggaagtgctcttgttcataatgtcttcccacaatattatcttacctgaccatcacttaaattctcctatagtactaaataaattaatgtattactgcaaattgctcaatgtattgcctgggcctgattctccttggtttgagaaaacaagaggatggactaattgctgtatccgtctttctgactgcaaccgcttaactctagcacgcgcctcaagaattagagatcaattagcaacaatgggaatatattcaaagaatcaatcaacatgttttaaaacaattattcatccacaatccttgcaaccaattatgcatagtgcatcagaattaggacggactctacctacatggtcgcgaatgagaagcgaggtgtcatacagtgtaacaacacaatcagcaaaatttggagacctattccaaggcatatctactgatctaacagggaagacaaatttgtttggcggattctgcgatttaaatcactcccttagcccacctgcacatgcattaatgactaagcctgggatgtatctagagactagtgatgcttacgcttgccaatttttgttccacattaaaacttgtcaacgagagttgatcttactcatgaggcaaaatgcaacagccgaactgattaagcaattccagtatccaggattgacaattataaccacacctgaatattcagtttgggtcttccatgaaagcaaacaagtcactatccttacttttgattgccttttaatgtactgtgatctcgctgatgggcgtcacaatatcctctttacatgccaattacttccgcacttaaatcatctaggtataaggatccgagacctcttagggctaatagataatctcgggaagaatcatcccttgattgtgtatgatgttgttgctagtttagaatcattggcatatggggccatacaactccatgacaaagttgttgattatgcaggtaccttcttcactttcattctggctgagatatatgaatctttagagtcctctctaccaagtggaaatagtgaagcgattgttactcaaattaggaacatatatacagggttaacagtaaatgaagcagctgagctcttatgtgtaatgagactctgggggcatcctgcattaagcagtatagatgcagcaaataaggtgcggcaaagtatgtgcgcagggaaactgttaaaatttgatacgatccaactggtattagccttcttcaatacgttaattatcaatggctatcgcaggaaacatcatggtaggtggccaaatgtggatagtaattcaatcttaggaacagatcttaagaggatgtattatgatcaatgtgaaatcccccatgagtttacacttaaacattatcatactgtgagtctaattgagtttgattgtacgtttccaatcgagctatccgacaaattaaacatatttcttaaagataaggcaattgcattccctaagtcaaagtggacatctccttttaaagccgatatcacacctaaacaattactcatccctcccgaatttaaagttcgtgcaaatcgccttctcttgactttcctgcagttagatgagttttctatcgaatcagaattagaatatgttacaaccaaagcatatctcgaagatgatgagttcaatgtatcatactctctcaaggagaaagaagtgaagacagatggtcgcatatttgctaaattaactcgtaagatgaggagttgtcaagtaatctttgaagagctccttgccgaacatgtgtccccccttttcaaagacaacggtgtaactatggctgaattatcattgaccaaaagcctacttgcaataagcaatttaagttccacattgtttgagacacaaacccgtcagggcgacagaaattcaagatttactcatgctcattttattacaactgacttacaaaagtactgtcttaattggagatatcaaagcgtgaagctctttgcacgccaattgaatcgtctattcgggttacagcatggttttgaatggatccattgtatcctcatgcagtccaccatgtatgtagctgatcccttcaatcctccaaacgggaacgcaagcccaaatttagatgataacccaaataatgacatctttattgtatcacctcgaggagcaattgagggcctgtgtcagaagatgtggacaattatatcaatctcagcaattcatgcagctgcagctgtagcaggcctaagagtcgcatcaatggttcaaggtgacaaccaggttatcggtgtcactcgagaattccttgcaggacatgatcaaagtcatgtggatagtcaacttactgcatcattagaaaactttacacaaatattcaaggagataaattatgggcttggccataacctcaaattacgggaaacaattaagtctagtcacatgttcatttattctaaaagaattttttacgatgggaggattctccctcaattgttaaagaatataagtaaactaactttgtcggcaactacaacaggggagaattgcttaactagctgtggggacttatcttcatgtattacccgctgtattgagaatggtttcccaaaggatgctgcattcattctaaatcagcttacaattaggactcagatacttgcagaccatttttactcaatacttggtgggtgcttcactgggctaaatcaacatgatattcgcttactgctctctgatggttctatattgccagctcagctggggggatttaacaacttgaatatatcccgattattctgtagaaatataggtgaccctctagtagcctcaattgcagatacaaaacgctatgtgaaatgcggccttttgactccatctatacttgactcagtcgtctccatcactgataggaaaggctcatttactaccctgatgatggatccctattcaatcaatctcgattatattcaacagccagaaacccgcttaaaacgtcatgtgcagaaagttctccttcaagaatcagtaaatcctctactgcagggcgtatttctcgagactcagcaggatgaagaggaagcactagctgcgtttttattagacagagatattgtgatgccccgtgtagctcacgcaatttttgaatgtacgagtctcggacgccgtagacacatacaggggctgattgatacaacaaagactataatagccctggcattggacacacagaatctgagtcacactaagcgtgagcaaatagttacgtataatgcaacctatatgaggtccttaacacaaatgcttaaattaagcagaactgttcataaggggatgaccaggatgctgcctattttcaatatcaatgattgttctgtaatactagcacaacaagttaggcgtgcaagctgggctccgctgctaaattggcgcaccttggaagggcttgaggtccctgatccaattgaatccgtgtctggataccttggtcttgactccaacaattgcttcctctgttgccatgaacaaaatagctactcttggtttttcctccccaaattgtgccattttgacgattcgagacaatcatactcaacccaacgtgtaccttatataggttcaaaaacagatgagagacaaatgtctacaattaacctcctagagaaaacaacctgtcatgcccgtgccgcaacaaggttagcgtcattatatatatgggcatatggtgattcggaagacagctgggatgcagtagaatcactatcaaatagccgatgccaaattacacgagagcaattgcaggccctttgccccatgccgtcatcagtaaatttacatcatagactcaatgacggtattacccaagttaagttcatgccatcaacaaacagcagagtatccagatttgtacatatttctaatgacaggcagaattacgtcctggacgacactgtcactgatagtaacttgatatatcagcaggtcatgcttttgggtttgagcatattggagacatactttcgagaaccaacaactgtgaacttgtcgagtatcgtcctccatttgcatactgacgtgtcctgttgtctccgtgaatgccctatgacacagtatgcaccaccactcagagacctccctgaactaaccataacaatgacaaatccattcctttatgaccaagcacctatcagtgaagcagatctatgtcggctttcgaaggtagccttccgtaaagcaggagacaattatgaactatatgatcaattccaactgcgatccacactctcttcaaccacagggaaggatgttgcggcaactatttttggaccacttgcggcagtatctgcaaaaaatgatgcaattgttactaatgactacagtggtaactggatctcagagttcaggtacagtgattactacctactgagtacgagtttgggttacgagattttactaatatttgcttaccaactctactatctaaggattaggtataagcaaaacatcatttgttacatggagtctgtattccgccgttgccactcattatgcttaggtgacctgattcaaacaatctcccactcagaaatactgactggattaaatgctgcaggcttcaacttgatgttggataggagtgatttgaagaataaccaattgtctcgcctagccgtcaagtatctcacgctctgtgtccaggctgccattaacaacttggaggttggctcagaacctctctgtattattggaggtcaactcgatgatgacatctcgtttcaggtagcgcattttctatgtagaaggctttgcattctaagtcttgtacactcaaatttacagaatctccccacgatccgtgataatgaggttgatgtgaaatctaaattaatttatgaccatctcaaactggttgctacaactttgaatgatcgagaccaatcgtatctgttaaagctgttaaataacccaaatttggaattacacacaccgcaagtctacttcataatgaggaagtgtctaggtttgctcaaggcgtatggcgcagtaccatacaaacaaccttttccaacatcacctattgtaccattccctaatctgagtgggtctaagtggcaccttgaacgtgttatagacagtattgaggcaccaaaatcttacacttgggttcctaacacaacactcccactggccaaggatcatgtatcccccaatccaagcagaattcttgacaaaatcaacttgtttagatcactgagccccagacactcagtttggtaccgtaatcgtcaatacaaacttatcctttcccagctgagtcatgatattcttgggggctctacactttacctaggtgaaggagggggctcaactatcctcacaattgaaccccacattagaagtgacaaaatatactaccatacatacttccctgccgatcagagtccggctcaacgcaactttataccccagcctacgacattcttgagatctaacttttatcactttgaactggaaccatcaggatgtgagtttgtaaattgctggtctgaggatgcaaacgccacaaatcttacagaacttaggtgtattaaccacatcatgacagtgataccagttggctcgttaaacagaatcatatgtgacatagagctagctagagacacatcaatcaagtcgatagccmcmgtttatcttaatctaggaattctagctcatgcattgcttagtccagggggaatctgcatatgcaggtgccatttactgaacgcttcaaatcttgcgattgtatcttttgtactaaaaacattgtcaagcaagctggcaatttcattctctggatttagcggtgtgaatgatccttcttgtgtggttggaactaccaaggaaagcactattagcttagatgttctcagttcaattgcttctgcattcataaacgaattgacatcgaatgaagtaccgattccccaagaggtattgacattactatcttgttacacagagcagctagggaacttagggcaattgattgagaaaacctggatccgcgagatacggaaaccgcatttaatgcagtgtgaaatggagtggatcgggcttttgggaaatgatgcattgagtgacgtagacaatttcctgaactattacaacccatcatgctcatcagttccagaactaattacacctacagttagttcattgctttttgaactggttagcctaactccagaagtctgctcttacgatgaatctaattataaacgaacaattcaggtagggcaggcatataacattacagtttctggcaaagtaagcactatgataaggacctgttgcgaacaatgcattaagcttctaatagctaatagtgaagtactaattgatactgatttggcgtatcttgttagaggcattcgcgatgggtcattcactctaggctcgatcataagccaaaaccaaatactaaaagcatccagagcaccacgttacctcaaaacacccaaaattcaattatgggtatcaacactgttagccattaggattgaggaagtcttctcacgccattatagaaaggtcctcttacgatcaatccgccttttgtcactctacaagtatctccaggacaagacgaagtagataaccatttatcatagagtcagacgggttctagttcaatccctgcgttattcttcgctcacagaatcttggattccatccggggctgtgctgacataatatgtaaatatgtaatatattggttactggacataatcaatgaggcttctgtagtatttatcccaactccttaatattagtttcaaaatgagaacattatatgttaataaaaaactaaaaatgataaccagttgaatctggaccgaactggcaattgcataaaaaataaaaaatttattaaaattaaaattgaaatcatataacaacacgtttaaggggaataaaaacaagattgggaataaaaataataataataaaaggaataaaacaaaaaataaaaataaaaatgggaataaaaataaaaataaaaataaagaaaaaaatgggagaaaagctccaattaacaaacaaatcaaaactaaacttaagattacaactaaaaatacaaatattaacaaaaatagactgagaagtagaatcgtaaataagaccggcagtcagtttagtatggaaaataagacccagattacttacacatcctgccttagtttcccccttatttaattttaagtggatttagggagtcactgatccagctaagaacctattttcttatagctaaaatctcaatcttgatgtctccaatcaattaaaaccggttgtttaattaagttgttcctaatcaattcacctcagtagatccagtgtgaatcgcactggtccaatccaacatgggtctaattaaataaaacgactgtaataggtcgaatgcggcctcgatcaacagagtaacaaacattacaaattacaaatcagagttgttaattaaaccatttatataactttttgtttagt

SEQ ID NO: 3 - DNA, 15054 bp, Avian paramyxovirus 4
gcgaaaaagaagaataaaaggcagaagccttttaaaaggaaccctgggctgtcgtaggtgtgggaaggttgtattccgagtgcgcctccgaggcatctactctacacctatcacaatggctggtgtcttctcccagtatgagaggtttgtggacaatcaatcccaagtatcaaggaaggatcatcggtccctggcagggggatgccttaaagtcaacatccctatgcttgtcactgcatctgaagatcccaccactcgttggcaactagcatgtttatctctaaggctcttgatctccaactcatcaaccagtgctatccgacagggggcaatactgactctcatgtcactaccgtcacaaaatatgagagcaacggcagctattgctggttccacaaatgcagctgttatcaacactatggaagtcttgagtgtcaatgactggaccccatccttcgaccctaggagcggtctctctgaagaggatgctcaggttttcagagacatggcaagggacctgccccctcagttcacctccggatcaccctttacatcagcattggcggaggggtttaccccagaagacacccacgacctaatggaggccttgaccagtgtgctgatacagatctggatcctggtggctaaggccatgaccaacattgatggctctggggaggccaatgagagacgtcttgcaaagtacatccaaaagggacagcttaatcgccagtttgcaattggtaatcctgctcgtctgataatccaacagacgatcaaaagctccttaactgtccgcaggttcttggtctctgagcttcgtgcatcacgaggtgcagtgaaagaaggatccccttactatgcagctgttggggatatccacgcttacatctttaacgcaggactgacaccattcttgactaccttaagatatgggataggcaccaagtatgctgctgttgcactcagtgtgttcgctgcagacattgcaaaattaaagagcctacttaccctgtaccaagacaagggtgtggaggccggatacatggcactccttgaagatccagattccatgcactttgcacccggaaatttcccacacatgtactcctatgcgatgggggtggcttcttaccatgaccccagcatgcgccaataccaatatgccaggaggttcctcagccgtcccttctacttgctagggagggacatggccgccaagaacacaggcacgctggatgagcaactggcaaaggaactgcaagtgtcagaaagagaccgcgccgcattgtccgctgcgattcaatcagcaatggaggggggagaatctgacgacttcccactgtcgggatccatgccggctctctccgacactgcgcaaccagttaccccaagaacccaacagtcccagctttcccctccacaatcatcaagcatgtctcaatcagcgcccaggaccccggactaccagcctgattttgaactgtaggctgcatccacgcaccaacaacaggcaaaagaaatcaccctcctccccacacatcccacccactcacccgccgagatccaatccaacaccccagcatccccatcatttaattaaaaactgaccaatagggtggggaaggagagttattggctgttgccaagtttgtgcagcaatggatttcaccgacattgatgctgtcaactcattaattgaatcatcatcagcaatcatagattccatacagcatggagggctgcaaccatcgggcactgtcggcctatcgcaaatcccaaaggggataaccagcgctttaactaaggcctgggaggctgaggcagcaactgctggcaatggggacacccaacacaaacctgacagtccggaggatcatcaggccaacgacacagactcccccgaagacacaggcaccaaccagaccatccaggaagccaatatcgttgaaacaccccaccccgaagtgctatcggcagccaaagccagactcaagaggcccaaggcagggagggacacccacgacaatccctctgcgcaacctgatcattttttaaaggggggccccctgagcccacaaccagcggcaccatgggtgcaaagtccacccattcatggaggtcccggcaccgtcgatccccgcccatcacaaactcaggatcattccctcaccggagagaaatggcaatcgtcaccgacaaagcaaccggagacattgaactggtggaatggtgcaacccggggtgcaccgcaatccgaactgaaccaaccagactcgactgtgtatgcggacactgccccaccatctgcagcctctgcatgtatgacgactgatcaggtacaactattaatgaaggaggttgccgatatgaaatcactccttcaggcattagtaaagaacctagctgtcctgcctcaactaaggaacgaggttgcagcaatcaggacatcacaggccatgatagaggggacactcaattcaatcaagattctcgatcctgggaattatcaagaatcatcactaaacagctggttcaaaccacgccaagatcacgcggttgttgtgtccggaccagggaatccattgaccatgccaaccccaatccaagacaacaccatattcctggatgaactggcaagacctcatcctagtttggtcaatccgtccccgcccactaccaacactaatgttgatcttggcccacagaagcaggctgcgatagcttatatctcagcaaaatgcaaggatccagggaaacgagatcagctctcaaagctcatcgagcgagcaaccaccttgagcgagatcaacaaagtcaaaagacaggccctcggcctctagatcactcgaccacccccagtaatgaatacaacaataatcagaacccccctaaaacacatggtcaacccaacacaccacccgcaccacccgctactatcctttgccagaaactccgccgcagccgatttattcaaaagaagccatttgatatgacttagcaaccgcaagatagggtggggaaggtgctttgcctgcaagagggctccctcatcttcagacacgtacccgccaacccaccagtgacgcaatggcagacatggacaccgtatatatcaatctgatggcagatgatccaacccaccaaaaagaactgctgtcctttcccctcgttcccgtgactggtcctgacgggaaaaaggaactccaacaccaggtccggactcaatccttgctcgcctcagacaagcaaactgagaggttcatcttcctcaacacttacgggtttatctatgacactacaccggacaagacaactttttccaccccagagcacatcaatcagcccaagagaacgatggtgagtgctgcgatgatgaccattggcctggtccccgccaatatacccttgaacgaattaacagctactgtgttcggcctgaaagtaagagtgaggaagagtgcgagatatcgagaggtggtctggtatcagtgcaatcctgtaccagccctgcttgcagccaccaggttcggtcgccaaggaggtctcgaatcaagcactggagtcagcgtaaaggcccccgagaagatagattgcgagaaggattatacttactacccttatttcctatctgtgtgctacatcgccacttccaacctgttcaaggtaccaaaaatggttgctaatgcgaccaacagtcaattataccacctgactatgcaggtcacatttgcctttccaaaaaacatccccccagctaaccagaaacttctgacacaagtggatgaaggattcgagggcactgtggactgccattttgggaacatgctgaaaaaggatcggaaagggaatatgaggacattgtcgcaggcggcagacaaggtcagacggatgaatatccttgttggtatctttgacttgcatgggccgacactcttcctggagtatactgggaaactaacaaaagctctgttagggttcatgtctactagccgaacagcaatcatccccatatctcagctcaatcctatgctgggtcaacttatgtggagcagtgatgcccagatagtaaaattaagagtggtcataactacatccaaacgcggcccatgcgggggtgagcaggagtatgtgctggatcccaaattcacagttaaaaaagagaaagcccgactcaaccctttcaagaaggcagcccaatgatcaaatctgcaggatctcaagaatcagaccactctatactattcaccgatcaatagacatgtaactatacagttgatggacctatgaagaatcaattagcaaaccgaatccttactagggtggggaaggagttgattgggtgtctaaacaaaagcattcctttacacctcctcgctacgaaacaaccataatgaggttatcacgcacaatcctgactttgattctcagcacacttaccggctatttaatgaatgcccactccaccaatgtgaatgagaaaccaaagtctgaggggattaggggggatcttataccaggcgcaggtatttttgtaactcaagtccgacaactacagatctaccaacagtctgggtatcatgaccttgtcatcaggttattacctcttctaccggcagaacttaatgattgtcaaagggaagttgtcacagagtacaacaacacggtatcacagctgttgcagcctatcaaaaccaacctggataccttattggctgatggtagcacaagggatgccgatatacagccacggttcattggggcaataatagccacaggtgccctggcggtggctacggtagctgaggtgactgcagcccaagcactatctcagtcgaaaacaaacgctcaaaatattctcaagttgagagatagtattcaggctaccaaccaagcagttttcgaaatttcacaaggactcgaggcaactgcaactgtgctatcaaaactgcaaactgagctcaatgagaacattatcccaagcctgaacaacttgtcctgtgctgccatggggaatcgccttggtgtatcactatcactctacttgaccttaatgaccactctatttggggaccagatcacaaacccagtgctgacaccaatctcctatagcactctatcggcaatggcaggcggtcacattggcccggtgatgagtaaaatattagctggatctgtcacaagtcagttgggggcagaacagttgattgctagcggcttaatacagtcacaggtagtaggttatgattcccaatatcaattattggttatcagggtcaaccttgtacggattcaagaggtccagaatacgagggtcgtatcactaagaacactagcggtcaatagggatggtggactttatagagcccaggtgcctcccgaggtagttgaacggtctggcattgcagagcgattttatgcagatgattgtgttcttactacaactgattacatttgctcatcgatccgatcttctcggcttaatccagagttagtcaagtgtctcagtggtgcacttgattcatgcacatttgagagggaaagtgcattattgtcgacccctttctttgtatacaacaaggcagtcgtcgcaaattgtaaagcagcaacatgtagatgtaataaaccgccatctattattgcccaatactctgcatcagctctagtcaccatcaccaccgacacctgtgccgaccttgaaattgagggttatcgcttcaacatacagactgaatccaactcatgggttgcaccaaacttcacggtctcgacttcacagattgtatcagttgatccaatagacatctcctctgacattgccaaaatcaacagttccatcgaggctgcgagagagcagctggaactgagcaaccagatcctttcccggatcaacccacgaattgtgaatgatgaatcactgatagctattatcgtgacaattgttgtgcttagtctccttgtaatcggtctgattgttgttctcggtgtgatgtataagaatcttaagaaagtccaacgagctcaagctgccatgatgatgcagcaaatgagctcatcacagcctgtgaccactaaattagggacgcctttctaggagaataatcatatcactctactcaatgatgagcaaaacgtaccaatcgtcaatgattgtgtcacgaggccggttgggaatgcatcgaatctctcccctttctttttaattaaaaacatttgaagtgagggtgagagggggggagtgtatggtagggtggggaaggtagccaattcctgcctattgggccgaccgtatcaaaagaactcaacagaagtctagatacagggtgacatggagggcagccgtgataatcttacagtggatgatgaattaaagacaacatggaggttagcttatagagttgtgtcccttctattgatggtgagcgctttgataatctctatagtaatcctgacaagagataacagccaaagcataatcacagcgatcaaccagtcatccgacgcagactcaaagtggcaaacgggaatagaagggaaaatcacctccattatgactgatacgctcgataccaggaatgcagcccttctccacattccactccagctcaacacgcttgaggcgaaccttttgtccgcccttgggggcaacacaggaattggtcccggggatctagatcactgccgttaccctgttcatgactccgcttacctgcatggagttaatcgattactcatcaaccagacagctgattacacagcagaaggccccctagatcatgtgaactttattccagccccggttacgaccactggatgcacaaggataccatccttttccgtgtcatcgtccatttggtgctatacacacaacgtgatcgaaaccggttgcaatgaccactcaggtagtaaccaatatatcagcatgggagtcattaagagagcgggcaacggcctaccttacttctcgacagttgtaagtaaatatctgactgatgggttgaataggaaaagctgttctgtagccgccggatccgggcattgctacctcctttgcagcttagtgtcggaacccgaacctgatgactatgtgtcacctgatcccacaccgatgaggttaggggtgctaacgtgggatgggtcttacactgaacaggtggtacccgaaagaatattcaagaacatatggagtgcaaactacccaggagtagggtcaggtgctatagtagggaataaggtgttattcccattttacggcggagtgagaaatggatcgaccccggaggtgatgaataggggaagatactactacatccaggatccaaatgactattgtcctgacccgctacaagatcagatcttaagggcggaacaatcgtattacccaactcgatttggtaggaggatggtaatgcaaggggtcctagcatgtccagtatccaacaattcaacaatagcaagccaatgtcaatcttactattttaataactcattaggattcattggggcagaatctagaatctattacctcaatggtaacatttacctttatcagagaagctcgagctggtggcctcatccccagatttacctgcttgattccaggattgcaagtccgggtactcagaacattgactcaggtgttaatctcaagatgttaaatgttactgtgattacacgaccatcatctggtttttgtaatagtcagtcacgatgccctaatgactgcttattcggggtctactcggatatctggcctcttagccttacctcagatagcatattcgcgttcacaatgtatttacaggggaagacaacacgtattgacccggcttgggcactattctccaatcatgcgattgggcatgaggctcgtctgttcaataagraggttagtgctgcttattctaccaccacttgtttttcggacactatccaaaatcaggtgtattgcctgagtatacttgaggtcaggagtgagctcttgggagcattcaaaatagtaccattcctctatcgcgtcttgtaggcatccattcagccaaaaaacttgagtgaccatgaggttaacacctgatccccttcaaaaacatctatcttaattaccgttctagatccatgattaggtacctttccaatcaatcatttggtttttaattaaaaacgaaagaatgggcctagttccaagaaagggctggaacccattagggtggggaaggattgctttgctccttgactcacacctgcgtacactcgatctcacttctataaagaaggaatccttctcaaattcgccccacaatgtccaatcaggcagctgagattatactacccaccttccatctagaatcacccttaatcgagaataagtgcttctattatatgcaattacttggtctcgtgttgccacatgatcactggagatggagggcattcgttaactttacagtggatcaggtgcaccttaaaaatcgtaatccccgcttaatggcccacatcgaccacactaaagatagattaaggactcatggtgtcttaggtttccaccagactcagacaagtatgagccgttaccgtgttttgcttcatcctgaaaccttaccttggctatcagccatgggaggatgcatcaatcaggttcctaaagcatggcggaacactctgaaatcgatcgagcacagtgtaaagcaggaggcacctcaactaaagttactcatggagagaacctcattaaaattaactggagtaccttacttgttctctaattgcaatcccgggaaaaccacagcaggaactatgcctgtcctaagtgagatggcatcggaactcttatcaaatcctatctcccaattccaatcaacatgggggtgtgctgcttcggggtggcaccatgtagtcagtatcatgaggctccaacaatatcaaagaaggacaggtaaggaagagaaagcaatcactgaagttcagtatggcacggacacctgtctcattaacgcagactacaccgttgttttttccacacagaaccgtgttataacggtcttgcctttcgatgttgtcctcatgatgcaagacctgctagaatcccgacggaatgtcctgttctgtgcccgctttatgtatcccagaagccaacttcatgagaggataagtacaatattagcccttggagaccaactggggagaaaagcaccccaagtcctgtatgattttgtagcaacccttgagtcatttgcatacgcagctgttcaacttcatgacaacaatcctacctacggtggggccttctttgaattcaatatccaagagttagaatctattctgtcccctgcacttagtaaggatcaggtcaacttctacataggtcaagtttgctcagcgtacagtaaccttcctccatctgaatcggcagaattgctgtgcctgctacgcctgtggggtcatcccttgctaaacagccttgatgcagcaaagaaagtcagggaatctatgtgtgccgggaaggttctcgattacaacgccattcgactcgtcttgtctttttatcatacgttactaatcaatgggtatcggaagaagcacaagggtcgctggccaaatgtgaatcaacattcactcctcaacccgatagtgaggcagctttattttgatcaggaggagatcccacactctgttgcccttgagcactatttggatgtctcaatgatagaatttgagaaaacttttgaagtggaactatctgacagcctaagcatcttcctgaaggataagtcgatagctttggacaagcaagaatggtacagtggttttgtctcagaagtgactccgaagcacctgcgaatgtcccgtcatgatcgcaagtctaccaataggctcctgttagccttcattaactcccctgaattcgatgttaaggaagagcttaaatacttgactacgggtgagtacgctactgacccaaatttcaatgtctcttactcactcaaagagaaggaagtaaagaaagaagggcgcattttcgcaaaaatgtcacaaaagatgagagcatgccaggttatttgtgaagaattgctagcacatcatgtggctcctttgtttaaagagaatggtgttactcaatcggagctatccctgacaaaaaatttgttggctattagccaactgagttacaactcgatggccgctaaggtgcgattgctgaggccaggggacaagttcactgctgcacactatatgaccacagacctaaagaagtactgtctcaattggcggcaccagtcagtcaaactgttcgccagaagcctggatcgactgtttgggctagaccatgctttttcttggatacatgtccgtctcaccaacagcactatgtacgttgctgaccccttcaatccaccagactcagatgcatgcacaaacttagacgacaataagaacaccgggatttttattataagtgcacgaggtggtatagaaggcctccaacaaaaactatggactggcatatcaatcgcaattgcccaagcagcagcagccctcgaaggcttacgaattgctgctactctgcagggggataaccaagttttggcgattacaaaggagttcatgaccccagtcccggaggatgtaatccatgagcagctatctgaggcgatgtcccgatacaaaaggactttcacatacctcaattatttaatggggcatcagttgaaggataaggaaaccatccaatccagtgatttctttgtgtactccaaaagaatcttcttcaatggatcaatcttaagtcaatgcctcaagaacttcagtaaactcactactaatgccactacccttgctgagaacactgtggccggctgcagtgacatctcttcatgcattgcccgttgtgtggaaaacgggttgcctaaggatgccgcatatattcagaatataatcatgactcggcttcaactattgctagatcattactattcaatgcatggcggcataaactcagaattagagcagccaactttaagtatctctgttcgaaacgcgacctacttaccatctcaactaggcggttacaatcatttgaatatgacccgactattctgccgcaatatcggcgacccgcttaccagttcttgggcggagtcaaaaagactaatggatgttggccttctcagtcgtaagttcttagaggggatattatggagacccccgggaagtgggacattttcaacactcatgcttgatccgttcgcacttaacattgattacctgaggccgccagagacaattatccgaaaacacacccaaaaagtcttgttgcaagattgcccaaatcccctattagcaggtgtcgttgacccgaactacaaccaagaattagagctattagctcagttcttgcttgatcgggaaaccgttatccccagggctgcccatgccatctttgaattgtctgtcttgggaaggaaaaaacatatacaaggattggtagatactacaaaaacaattattcagtgctcattggaaagacagccattgtcctggaggaaagttgagaacattgttacctacaacgcgcagtatttcctcggggccacccaacaggctgatactaatgtctcagaagggcagtgggtgatgccaggtaacttcaagaagcttgtgtcccttgacgattgctcagtcacgttgtccactgtatcgcggcgcatatcgtgggccaatctactgaactggagagctatagatggtttagaaaccccggatgtgatagagagtattgatggccgccttgtacaatcatccaatcaatgtggcctatgtaatcaagggttgggatcctactcctggttcttcttgccctctgggtgtgtgttcgaccgtccacaagattctcgggtagttccaaagatgccatacgtggggtccaaaacagatgagagacagactgcatcagtgcaagctatacagggatccacttgtcacctcagagcagcattgaggcttgtatcactctatctatgggcctatggagattctgacatatcatggctagaagctgcgacactggctcaaacacggtgcaatgtttctcttgatgacttgcgaatcttgagccctctcccttcttcggcgaatttacaccacagattaaatgacggggtaacacaggttaaattcatgcccgccacatcgagccgagtgtcaaagttcgtccaaatttgcaatgacaaccagaatcttatccgtgatgatgggagtgttgattccaatatgatttatcaacaagttatgatattggggcttggagagattgaatgcttgctagctgacccaatcgatacaaacccagaacaattgattcttcatctacactctgataattcttgctgtctccgggagatgccaacgaccggctttgtacctgctctaggactaaccccatgtttaactgtcccaaagcacaatccttacatttatgatgatagcccaatacccggtgatttggaccagaggctcatccagaccaaatttttcatgggttctgacaatttggataatcttgatatctaccaacagcgggctttattgagtaggtgtgtggcttatgatgttatccaatcgatatttgcttgtgatgcaccagtctctcagaagaatgacgcaatccttcacactgactatcatgagaattggatctcagagttccgatggggtgaccctcgtattatccaagtaacggcaggctacgagttaattctgttccttgcataccagctttattatctcagagtgaggggtgaccgtgcaatcctatgttatattgacaggatactcaacaggatggtatcttccaatctaggcagtctcatccagacactctctcatccagagattaggaggagattctcattgagtgatcaagggttccttgttgaaagggagctagagccaggtaagcccttggttaaacaagcggttatgttcttgagggactcggtccgctgcgctttagcaactatcaaggcaggaattgagcctgagatctcccgaggtggctgtactcaggatgagctgagctttactcttaagcacttactgtgtcggcgtctctgtgtaatcgctctcatgcattcagaagcaaagaacttggttaaagttagaaaccttcctgtagaagagaaaaccgccttactgtaccagatgttggtcactgaggccaatgctaggaaatcaggatctgctagcatcatcataaatctagtctcggcaccccagtgggacattcatacaccagcattgtattttgtatcaaagaaaatgctagggatgcttaaaaggtcaaccacacccttggatataagtgacctctccgagagccagaatcccgcacttgcagagctgaatgatgttcccggtcacatggcagaagaatttccctgtttgtttagtagttataacgccacatatgaagacacaattacttacaatccaatgactgaaaaactcgccttacacttggacaacagttccaccccatccagagcacttggtcgtcactacatcctgcggcctcttgggctctactcatccgcatggtaccggtctgcagcactactagcgtcaggggccctaaatgggttgcctgaggggtcgagcctgtacctaggagaagggtacgggaccaccatgactctgcttgagcccgttgtcaagtcttcaactgtttactaccatacattgtttgacccaacccggaatccttcacagcggaactataaaccagaaccacgggtattcacggattctatttggtacaaggatgatttcacacggccacctggtggtattatcaatctgtggggtgaagatatacgtcagagtgatatcacacagaaagacacggtcaacttcatactatctcagatcccgccaaaatcacttaagttgatacacgttgatattgagttctcaccagactccgatgtacggacactactatctggctattctcattgtgcactattggcctactggctattgcaacctggagggcgatttgcagttagagttttcttaagtgaccatatcatagtaaacttggtcactgcaatcctgtctgcttttgactctaatctggtgtgcattgcatcaggattgacacacaaggatgatggggcaggttatatttgcgcaaaaaagcttgcaaatgttgaggcttcaaggatcgagtactacttgaggatggtccatggttgtgttgactcattaaagatccctcatcaattaggaatcattaaatgggccgagggcgaggtgtcccaacttaccagaaaggcggatgatgaaataaattggcggttaggtgatccagttaccagatcatttgatccagtttctgagctaataattgcacgaacaggggggtctgtattaatggaatacggggcttttactaacctcaggtgtgcgaacttggcagatacatacaaacttctggcttcaattgtagagaccaccctaatggaaataagggttgagcaagatcaattagaagataattcgaggagacaaatccaagtagttcccgctttcaacactagatctgggggaaggatccgtacgctgattgagtgtgctcagctgcagattatagatgttatttgtgtaaacatagatcacctctttcctaaacaccgacatgttcttgtcacacaacttacctaccagtcagtgtgccttggggacttgattgaaggcccccaaattaagacgtatctaagggccaggaagtggatccaacgtcagggactcaatgagacagttaaccatatcatcactggacaagtgtcgcggaataaagcaagggattttttcaagaggcgtctgaagttggttggcttttcactctgcggtggttggagctacctctcactttagctgttcaggttgttgattattatgaataatcggagtcggaatcgtaaataggaagtcacaaagttgtgaataaacaatgattgcattagtatttaataaaaaatatgtcttttatttcgt

SEQ ID NO: 4 - DNA, 15054 bp, Avian paramyxovirus 4
acgaaaaagaagaataaaaggcagaagccttttaaaaggaaccctgggctgtcgtaggtgtgggaaggttgtattccgagtgcgcctccgaggcatctactctacacctatcacaatggctggtgtcttctcccagtatgagaggtttgtggacaatcaatcccaagtgtcaaggaaggatcatcggtccttagcaggaggatgccttaaagttaacatccctatgcttgtcactgcatctgaagaccccaccactcgttggcaactagcatgcttatctctaaggctcctgatctccaactcatcaaccagtgctatccgtcagggggcaatactgactctcatgtcattaccatcacaaaacatgagagcaacagcagctattgctggttccacaaatgcagctgttatcaacaccatggaagtcttaagtgtcaacgactggaccccatccttcgaccctaggagcggtctttctgaggaagatgctcaagttttcagagacatggcaagagatctgccccctcagttcacctctggatcacccttcacatcagcattggcggaggggttcactcctgaagatactcatgacctgatggaggccttgaccagtgtgctgatacagatctggatcctggtggctaaggccatgaccaacattgacggctctggggaggccaatgaaagacgtcttgcaaagtacatccaaaaaggacagcttaatcgtcagtttgcaattggtaatcctgcccgtctgataatccaacagacaatcaaaagctccttaactgtccgtaggttcttggtctctgagcttcgtgcgtcacgaggtgcagtaaaagaaggatccccttactatgcagctgttggggatatccacgcttacatctttaatgcgggattgacaccattcttgaccaccttaagatacgggataggcaccaagtacgccgctgttgcactcagtgtgttcgctgcagatattgcaaagttgaagagcctacttaccctgtaccaggacaagggtgtagaagctggatacatggcactccttgaggatccagactccatgcactttgcacctggaaacttcccacacatgtactcctatgcaatgggggtagcttcttaccatgatcctagcatgcgccaataccaatacgccaggaggttcctcagccgtcctttctacttactaggaagggacatggccgccaagaacacaggcacgctggatgagcaactggcgaaggaactgcaagtatcagagagagatcgcgccgcattatccgctgcgattcaatcagcgatggaggggggagagtccgacgacttcccactgtcgggatccatgccggctctctctgagaatgcgcaaccagttacccccagacctcaacagtcccagctctctcccccccaatcatcaaacatgccccaatcagcacccaggaccccagactatcaacccgactttgaactgtaggcttcatcaccgcaccaacaacagcccaagaagaccacccctccccccacacatctcacccagccacccataaagactcagtcccacgccccagcatctccttcatttaattaaaaaccgaccaacagggtggggaaggagagtcattggctactgccaattgtgtgcagcaatggattttactgacattgatgctgtcaactcattgatcgaatcatcatcggcaatcatagactccatacagcatggagggctgcaaccagcgggcaccgtcggcctatcgcagatcccaaaagggataaccagcgcattaaccaaggcctgggaggctgaggcggcaactgccggtaatggggacacccaacacaaatctgacagtccggaggatcatcaggccaacgacacagattcccctgaagacacaggtactgaccagaccacccaggaggccaacatcgttgagacaccccaccccgaggtgctgtcagcagccaaagccagactcaagaggcccaaagcagggagggacacccgcgacaactcccctgcgcaacccgatcatcttttaaaggggggcctcctgagcccacaaccagcagcatcatgggtgcaaaatccacccagtcatggaggtcccggcaccgccgatccccgcccatcacaaactcaggatcattcccccaccggagagaaatggcgattgtcaccgacaaagcaaccggagacattgaactggtggagtggtgcaacccggggtgcacagcagtccgaattgaacccaccagactcgactgtgtatgcggacactgccccaccatctgtagcctctgcatgtatgacgactgatcaggtacaactactaatgaaggaggttgctgacataaaatcactccttcaggcgttagtgaggaacctcgctgtcttgccccaattgaggaatgaggttgcagcaatcagaacatcacaggccatgatagaggggacactcaattcgatcaagattcttgaccctgggaattatcaggaatcatcactaaacagttggttcaaacctcgccaagatcacactgttgttgtgtctggaccagggaatccattggccatgccaaccccagtccaagacaacaccatattcctggacgagctagccagacctcatcctagtgtggtcaatccttccccacccatcaccaacaccaatgttgaccttggcccacagaagcaggctgcaatagcctatatctccgctaaatgcaaggatccggggaaacgagatcagctatcaaggctcattgagcgagcaaccaccccaagtgagatcaacaaagttaaaagacaagcccttgggctctagatcactcgatcacccctcatggtgatcacaacaataatcagaacccttccgaaccacatgaccaacccagcccaccgcccacaccgtccatcgacatcccttgccaaacatcctgccgtagctgatttattcaaaagagctcatttgatatgacctggtaatcataaaatagggtggggaaggtgctttgcctgtaagggggctccctcatcttcagacacgtgcccgccatctcaccaacagtgcaatggcagacatggacacggtgtatatcaatctgatggcagatgacccaacccaccaaaaagaactgctgtcctttcctctcatccctgtgaccggtcctgacgggaagaaggaactccaacaccagatccggacccaatccttgctcgcctcagacaaacaaactgaacggttcatcttcctcaacacttacggattcatctatgacaccacaccggacaagacaactttttccaccccagagcatattaatcagcctaagaggacgacggtgagtgccgcgatgatgaccattggcctggttcccgccaatatacccctgaacgaactaacggctactgtgttcagccttaaagtaagagtgaggaaaagtgcgaggtatcgggaagtggtctggtatcaatgcaatccagtaccggccctgcttgcagccaccaggtttggtcgccaaggaggtctcgagtcgagcactggagtcagtgtaaaggctcccgagaagatagattgtgagaaggattatacctactacccttatttcttatctgtgtgctacatcgccacctccaacctgttcaaggtaccgaggatggttgctaatgcaaccaacagtcaattataccaccttaccatgcaggtcacatttgcctttccaaaaaacatccctccagccaaccagaaactcctgacacaggtggatgagggattcgagggcactgtggattgccattttgggaacatgctgaaaaaggatcggaaagggaacatgaggacactgtcccaggcggcagataaggtcagacgaatgaatattcttgttggtatctttgacttgcatgggccaacgctcttcctggagtataccgggaaactgacaaaggctctgctagggttcatgtccaccagccgaacagcaatcatccccatatctcagctcaatcccatgctgagtcaactcatgtggagcagtgatgcccagatagtaaagttaagggttgtcataactacatccaaacgcggcccgtgcgggggtgagcaggagtatgtgctggatcccaaattcacagttaagaaagaaaaggctcgactcaaccctttcgagaaggcagcctaatgatttaatccgcaagatcccagaaatcagaccactctatactatccactgatcactggaaatgtaattgtacagttgatgaatctgtgaagaatcaattaaaaaaccggatccttattagggtggggaagtagttgattgggtgtctaaacaaaagcatttcttcacacctccccgccacgaaacaaccacaatgaggctatcaaacacaatcttgaccttgattctcatcatacttaccggctatttgataggtgtccactccaccgatgtgaatgagaaaccaaagtccgaagggattaggggtgatcttacaccaggtgcgggtattttcgtaactcaagtccgacagctccagatctaccaacagtctgggtaccatgatcttgtcatcagattgttacctcttctaccaacagagcttaatgattgtcaaagggaagttgtcacagagtacaataacactgtatcacagctgttgcagcctatcaaaaccaacctggatactttgttggcagatggtagcacaagggatgttgatatacagccgcgattcattggggcaataatagccacaggtgccctggctgtagcaacggtagctgaggtaactgcagctcaagcactatctcagtcaaaaacgaatgctcaaaatattctcaagttgagagatagtattcaggccaccaaccaagcagtttttgaaatttcacagggactcgaagcaactgcaaccgtgctatcaaaactgcaaactgagctcaatgagaatatcatcccaagtctgaacaacttgtcctgtgctgccatggggaatcgccttggtgtatcactctcactctatttgaccttaatgaccactctatttggggaccagatcacaaacccagtgctgacgccaatctcttacagcaccctatcggcaatggcgggtggtcacattggtccagtgatgagtaagatattagccggatctgtcacaagtcagttgggggcagaacaactgattgctagtggcttaatacagtcacaggtagtaggttatgattcccagtatcagctgttggttatcagggtcaaccttgtacggattcaggaagtccagaatactagggttgtatcactaagaacactagcagtcaatagggatggtggactttacagagcccaggtgccacccgaggtagttgagcgatctggcattgcagagcggttttatgcagatgattgtgttctaactacaactgattacatctgctcatcgatccgatcttctcggcttaatccagagttagtcaagtgtctcagtggggcacttgattcatgcacatttgagagggaaagtgcattactgtcaactcccttctttgtatacaacaaggcagtcgtcgcaaattgtaaagcagcgacatgtagatgtaataaaccgccatctatcattgcccaatactctgcatcagctctagtaaccatcaccaccgacacttgtgctgaccttgaaattgagggttatcgtttcaacatacagactgaatccaactcatgggttgcaccaaacttcacggtctcaacctcacaaatagtatcggttgatccaatagacatatcctctgacattgccaaaattaacaattctatcgaggctgcgcgagagcagctggaactgagcaaccagatcctttcccgaatcaacccacggattgtgaacgacgaatcactaatagctattatcgtgacaattgttgtgcttagtctccttgtaattggtcttattattgttctcggtgtgatgtacaagaatcttaagaaagtccaacgagctcaagctgctatgatgatgcagcaaatgagctcatcacagcctgtgaccaccaaattggggacacccttctaggtgaataatcatatcaatccattcaataatgagcgggacataccaatcaccaacgactgtgtcacaaggccggttaggaatgcaccggatctctctccttcctttttaattaaaaacggttgaactgagggtgagggggggggtgtgcatggtagggtggggaaggtagccaattcctgcccattgggccgaccgtaccaagagaagtcaacagaagtatagatgcagggcgacatggagggtagccgtgataacctcacagtagatgatgaattaaagacaacatggaggttagcttatagagttgtatccctcctattgatggtgagtgccttgataatctctatagtaatcctgacgagagataacagccaaagcataatcacggcgatcaaccagtcgtatgacgcagactcaaagtggcaaacagggatagaagggaaaatcacctcaatcatgactgatacgctcgataccaggaatgcagctcttctccacattccactccagctcaatacacttgaggcaaacctgttgtccgccctcggaggttacacgggaattggccccggagatctagagcactgtcgttatccggttcatgactccgcttacctgcatggagtcaatcgattactcatcaatcaaacagctgactacacagcagaaggccccctggatcatgtgaacttcattccggcaccagttacgactactggatgcacaaggatcccatccttttctgtatcatcatccatttggtgctatacacacaatgtgattgaaacaggttgcaatgaccactcaggtagtaatcaatatatcagtatgggggtgattaagagggctggcaacggcttaccttacttctcaacagtcgtgagtaagtatctgaccgatgggttgaatagaaaaagctgttccgtagctgcgggatccgggcattgttacctcctttgtagcctagtgtcagagcccgaacctgatgactatgtgtcaccagatcccacaccgatgaggttaggggtgctaacaagggatgggtcttacactgaacaggtggtacccgaaagaatatttaagaacatatggagcgcaaactaccctggggtagggtcaggtgctatagcaggaaataaggtgttattcccattttacggcggagtgaagaatggatcaacccctgaggtgatgaataggggaagatattactacatccaggatccaaatgactattgccctgacccgctgcaagatcagatcttaagggcagaacaatcgtattatcctactcgatttggtaggaggatggtaatgcagggagtcctaacatgtccagtatccaacaattcaacaatagccagccaatgccaatcttactatttcaacaactcattaggattcatcggggcggaatctaggatctattacctcaatggtaacatttacctttatcaaagaagctcgagctggtggcctcacccccaaatttacctacttgattccaggattgcaagtccgggtacgcagaacattgactcaggcgttaacctcaagatgttaaatgttactgtcattacacgaccatcatctggcttttgtaatagtcagtcaagatgccctaatgactgcttattcggggtttattcagatgtctggcctcttagccttacctcagacagcatatttgcatttacaatgtacttacaagggaagacgacacgtattgacccagcttgggcgctattctccaatcatgtaattgggcatgaggctcgtttgttcaacaaggaggttagtgctgcttattctaccaccacttgtttttcggacaccatccaaaaccaggtgtattgtctgagtatacttgaagtcagaagtgagctcttgggggcattcaagatagtgccattcctctatcgtgtcttataggcacctgcttggtcaagaaccctgagcagccataaaattaacacttgatcttccttaaaaacacctatctaaattactgtctgagatccctgattagttaccctttcaatcaatcaattaatttttaattaaaaacggaaaaatgggcctagttccaaggaaaggatgggacccattagggtggggaaggattactttgttccttgactcgcacccacgtacacccaatcccattcctgtcaagaaggaacccttcccaaactcaccttgcaatgtccaatcaggcagctgagattatactacccaccttccatcttttatcacccttgatcgagaataagtgcttctactacatgcaattacttggtctcgtgttaccacatgatcactggagatggagggcattcgtcaattttacagtggatcaagcacaccttaaaaatcgtaatccccgcttaatggcccacatcgatcacactaaggatagactaagggctcatggtgtcttgggtttccaccagactcagacaagtgagagccgtttccgtgtcttgctccatcctgaaactttaccttggctatcagcaatgggaggatgcatcaaccaggttcccaaggcatggcggaacactctgaaatctatcgagcacagtgtgaagcaggaggcgactcaactgaagttactcatggaaaaaacctcactaaagctaacaggagtatcttacttattctccaattgcaatcccgggaaaactgcagcgggaactatgcccgtactaagtgagatggcatcagaactcttgtcaaatcccatctcccaattccaatcaacatgggggtgtgctgcttcagggtggcaccatgtagtcagcatcatgaggctccaacagtatcaaagaaggacaggtaaggaagagaaagcaatcactgaagttcagtatggctcggacacctgtctcattaatgcagactacaccgtcgttttttccgcacaggaccgtgtcatagcagtcttgcctttcgatgttgtcctcatgatgcaagacctgcttgaatcccgacggaatgtcttgttctgtgcccgctttatgtatcccagaagccaactacatgagaggataagtacaatactggcccttggagaccaactcgggagaaaagcaccccaagtcctgtatgatttcgtagctaccctcgaatcatttgcatacgctgctgtccaacttcatgacaacaaccctatctacggtggggctttctttgagttcaatatccaagaactggaagctattttgtcccctgcacttaataaggatcaagtcaacttctacataagtcaagttgtctcagcatacagtaaccttcccccatctgaatcagcagaattgctatgcttactacgcctgtggggtcatcccttgctaaacagtcttgatgcagcaaagaaagtcagagaatctatgtgtgctgggaaggttcttgattataatgctattcgactagttttgtctttttatcatacgttattaatcaatgggtatcggaagaaacataagggtcgctggccaaatgtgaatcaacattcactactcaacccgatagtgaagcagctttactttgatcaggaggagatcccacactctgttgcccttgagcactatttagatatctcgatgatagaatttgagaagacttttgaagtggaactatctgatagtctaagcatctttctgaaggataagtcgatagctttggataaacaagaatggcacagtggttttgtctcagaagtgactccaaagcacctacgaatgtctcgtcatgatcgcaagtctaccaataggctattgttagcctttattaactcccctgaattcgatgttaaggaagagcttaaatatttgactacaggtgagtatgccactgacccaaatttcaatgtctcttactcactgaaagagaaggaagttaagaaagaagggcgcattttcgcaaagatgtcacagaaaatgagagcatgccaggttatttgtgaagagttactagcacatcatgtggctcctttgtttaaagagaatggtgttacacaatcggagctatccctgacaaagaatttgttggctattagccaactgagttacaactcgatggccgctaaggtgcgattgctgaggccaggggacaagttcaccgctgcacactatatgaccacagacctaaaaaagtactgccttaactggcggcaccagtcagtcaaattgttcgccagaagcctggatcgactatttgggttagaccatgctttttcttggatacacgtccgtctcaccaatagcactatgtacgttgctgacccattcaatccaccagactcagatgcatgcacaaatttagacgacaataagaacactgggatttttattataagtgctcgaggtggtatagaaggccttcaacagaaactatggactggcatatcaattgcaatcgcccaggcggcagcagccctcgagggcttacgaattgctgccactttgcagggggataaccaggttttagcgattacgaaagaattcatgaccccagtctcggaggatgtaatccacgagcagctatctgaagcgatgtcgcgatacaagaggactttcacataccttaattatttaatggggcaccaattgaaggataaagaaaccatccaatccagtgacttcttcgtttactccaaaaggatcttcttcaatgggtcaatcctaagtcaatgcctcaagaacttcagtaaactcactaccaatgccactacccttgctgagaacactgtagccggctgcagtgacatctcctcatgcatagcccgttgtgtggaaaacgggttgcctaaggatgctgcatatgttcagaatataatcatgactcggcttcaactgttgctagatcactactattctatgcatggtggcataaactcagagttagagcagccaactctaagtatccctgtccgaaacgcaacctatttaccatctcaattaggcggttacaatcatttgaatatgacccgactattctgtcgcaatatcggtgacccgcttactagttcttgggcagagtcaaaaagactaatggatgttggccttctcagtcgtaagttcttagaggggatattatggagacccccgggaagtgggacattttcaacactcatgcttgatccgttcgcacttaacattgattacttaaggccaccagagacaataatccgaaaacacacccaaaaagtcttgttgcaggattgtcctaatcctctattagcaggtgtagttgacccgaactacaaccaggaattagaattattagctcagttcctgcttgatcgggaaaccgttattcccagggctgcccatgccatctttgaactgtctgtcttgggaaggaaaaaacatatacaaggattggttgatactacaaaaacaattattcagtgctcattagaaagacagccactgtcctggaggaaagttgagaacattgtaacctacaatgcgcagtatttcctcggggccacccagcaggttgacaccaatatctcagaaaggcagtgggtgatgccaggtaatttcaagaagcttgtatctcttgacgattgctcagtcacgttgtccactgtgtcacggcgcatttcttgggccaatctacttaactggagggctatagatggtttggaaactccagatgtgatagagagtattgatggccgccttgtgcaatcatccaatcaatgcggcctatgtaatcaaggattgggctcctactcctggttcttcttgccctccgggtgtgtgttcgaccgtccacaagattctcgagtggttccaaagatgccatacgtgggatccaaaacggatgagagacagactgcgtcagtgcaggctatacagggatccacatgtcaccttagagcagcattgagacttgtatcactctacctttgggcctatggagattctgacatatcatggctagaagccgcgacattggctcaaacacggtgcaatatttctcttgatgacctgcggatcctgagccctcttccttcctcggcaaatttacaccacagattgaatgacggggtaacacaagtgaaattcatgcccgccacatcgagccgggtgtcaaagttcgtccaaatttgcaatgacaaccagaatcttatccgtgatgatgggagtgttgattccaatatgatttatcagcaggttatgatattagggcttggagagattgaatgtttgttagctgacccaatcgatacaaacccagaacaactgattcttcacctacactctgataattcttgctgtctccgggagatgccaacgaccggttttgtacctgctttaggattgaccccatgcttaactgtcccaaagcacaatccgtatatttatgatgatagcccaatacccggtgatttggatcagaggctcattcaaaccaaattctttatgggttctgacaatctagataatcttgatatctaccagcagcgagctttactgagtcggtgtgtggcttatgacattatccaatcagtattcgcttgcgatgcaccagtatctcagaagaatgatgcaatccttcacactgactaccatgaaaattggatctcagagttccgatggggtgaccctcgcataatccaagtaacagcaggttacgagttaattctgttccttgcataccagctttattatctcagagtgaggggtgaccgtgcaatcctgtgttatattgataggatactcaacaggatggtatcttccaatctaggcagtctcatccagacgctctctcatccggagattaggaggagattttcattgagtgatcaagggttccttgtcgaaagggagctagagccaggtaagccactggtaaaacaagcggttatgttcctaagggactcagtccgctgcgctttagcaactatcaaggcaggaattgagcctgagatctcccgaggtggctgtacccaggatgagctgagctttacccttaagcacttactatgtcggcgtctctgtataattgctctcatgcattcggaagcaaagaacttggtcaaagttagaaaccttccagtagaggaaaaaaccgccttactataccagatgttgatcactgaggccaatgccaggagatcagggtctgctagtatcatcataagcttagtttcagcaccccagtgggacattcatacaccagcgttgtattttgtatcaaagaaaatgctggggatgctcaaaaggtcaaccacacccttggatataagtgacctttctgagagccagaacctcacaccaacagaattgaatgatgttcctggtcacatggcagaggaatttccctgtttgtttagcagttataacgctacatatgaagacacaattacttacaatccaatgactgaaaaactcgcagtgcacttggacaatggttccaccccttccagagcgcttggtcgtcactacatcctgcgaccccttgggctttactcgtctgcatggtaccggtctgcagcactattagcgtcaggggccctcagtgggttgcctgaggggtcaagcctgtacttgggagaggggtatgggaccaccatgactctacttgagcccgttgtcaagtcctcaactgtttactaccatacattgtttgacccaacccggaatccttcacagcggaactacaaaccagaaccgcgggtattcactgattccatttggtacaaggatgatttcacacgaccacctggtggcattgtaaatctatggggtgaagacgtacgtcagagtgatattacacagaaagacacggttaatttcatattatctcgggtcccgccaaaatcactcaaattgatacacgttgatattgagttctccccagactctgatgtacggacgctactatctggctattcccattgtgcactattggcctactggctactgcaacctggagggcgatttgcggttagagttttcttaagtgaccatatcatagtcaacttggtcactgccattctgtccgcttttgactctaatctggtgtgcattgcgtcaggattgacacacaaggatgatggggcaggttatatttgtgcaaagaagcttgcaaatgttgaggcttcaagaattgagtattacttgaggatggtccacggctgtgttgactcattaaaaattcctcatcaattaggaatcattaaatgggctgagggtgaagtgtcccgacttaccaaaaaggcggatgatgaaataaactggcggttaggtgatccagttaccagatcatttgatccggtttctgagctaataattgcgcgaacagggggatcagtattaatggaatacgggacttttactaacctcaggtgtgcgaacttggcagatacatataaacttttggcttcaattgtagagaccaccttaatggaaataagggttgagcaagatcagttggaagatgattcgaggagacaaatccaggtagtccctgcttttaatacaagatccgggggaaggatccgtacattgattgagtgtgctcagctgcaggtcatagatgttatctgtgtgaacatagatcacctctttcccaaacaccgacatgctcttgtcacacaacttacttaccagtcagtgtgccttggggacttgattgaaggcccccaaattaagacatatctaagggccaggaagtggatccaacgtaggggactcaatgagacaattaaccatatcatcactggacaagtgtcgcggaataaggcaagggattttttcaagaggcgcctgaagttggttggcttttcgctctgtggcggttggggctacctctcactttagctgcttagattgttgattattatgaataatcggagtcgaaatcgtaaatagaaagacataaaattgcaaataagcaatgatcgtattaatatttaataaaaaatatgtcttttatttcgt

SEQ ID NO: 5 - DNA, 15054 bp, Avian paramyxovirus 4
acgaaaaagaagaataaaaggcagaagccttttaaaaggaaccctgggctgtcgtaggtgtgggaaggttgtattccgagtgcgcctccgaggcatctactctacacctatcacaatggctggtgtcttctcccagtatgagaggtttgtggataaccaatcccaagtgtcaaggaaggatcatcggtccctggcagggggatgcctcaaagtcaacatccctatgcttgtcactgcatctgaagatcccaccactcgttggcaactagcatgtttatctttaaggctcttgatctccaactcatcaaccagcgctatccgccagggggcaatactgactctcatgtcactaccatcacaaaatatgagagcaacggcagctattgctggttccacaaatgcagctgttatcaacactatggaagtcctaagtgtcaacgactggaccccatccttcgaccctaggagcggtctctctgaagaggatgctcaggtttttagagacatggcaagggatctgccccctcagttcacctccggatcaccctttacatcagctttggcggaggggtttaccccagaagacacccacgacctaatggaggccttgaccagtgtgctgatacagatctggatcctggtggctaaggccatgaccaacattgatggttctggggaggccaatgagagacgtcttgcaaagtatatccagaagggacagctcaatcgccagtttgcaattggtaatcctgctcgtctaataatccaacagacgatcaaaagctccttaactgtccgcaggttcttggtctctgagcttcgtgcatcacgaggtgcggtgaaagaaggatccccttattatgcagctgttggggatatccacgcatacatctttaacgcaggactgacaccattcttgactactttaagatatgggatcggcaccaagtatgctgctgttgcactcagtgtgttcgctgcagacattgcaaaattaaagagtctacttaccttataccaagataagggtgtggaggccggatacatggcactccttgaagatccagactccatgcactttgcacctggaaacttcccacacatgtactcctacgcgatgggggtggcttcttaccatgaccccagcatgcgccagtaccaatatgccaggaggttcctcagccgacccttctacttgctaggaagggacatggccgccaagaatacaggcacgctggatgagcaactggcaaaggaactgcaagtgtcagagagagaccgcgccgcactgtccgctgcgattcaatcagcaatggaagggggagaatccgacgacttcccactgtcgggatccatgccggctctctccgacaatgcacaaccagttaccccaagaacccaacagtcccagctctcccctccccaatcatcaagcatgtctcaatcagcgcccaggaccccggactaccagcctgattttgaactgtaggctgcatccatgcaccagcagcaggccaaagaaaccaccctcctctccacacatcccacccaatcacccgctgagactcaatccaacaccctagcatccccctcatttaattaaaaactgaccaatagggtggggaaggagagttattggctattgccaagttcgtgcagcaatggattttaccgatattgatgctgtcaactcattaatcgaatcatcatcagcaatcatagattccatacagcatggagggctgcaaccatcaggcactgtcggcctatcgcaaatcccaaaggggataaccagcgctttaaccaaagcctgggaggctgaggcagcaaatgctggcaatggggacacccaacaaaagtctgacagtctggaggatcatcaggccaacgacacagactcccccgaagacacaggcactaaccagaccatccaggaaaccaatatcgttgaaacaccccaccccgaagtgctatcggcagccaaagccagactcaagaggcccaaggcagggaaggacacccacgacaatccctctgcgcaacctgatcatcttttaaaggggggccccttgagcccacaaccagtggcaccgtgggtgcaaaatccgcccattcatggaggtcccggcaccgccgatccccgcccatcacaaactcaggatcattccctcaccggagagagatggcaatcgtcaccgacaaagcaaccggagccatcgaactggtggaatggtgcaacccggggtgcacagcaatccgaattgaacctaccagactcgactgtgtatgcggacactgccccaccatctgcagcctctgcatgtatgacgactgatcaggtacaactattaatgaaggaggttgccgatatgaaatcactccttcaggcactagtgaggaacctagctgtcctgcctcaactaaggaacgaggttgcagcaatcaggacatcacaggctatgatagaggggacacttaattcaatcaagattctcgaccctgggaattatcaggaatcatcactaaacagttggttcaaaccacgacaagatcacgcggttgttgtgtccggaccagggaatccattgaccatgccaaccccaatccaggacaataccatattcctggatgaattggcaagacctcatcctagtttggtcaatccgtccccgcccactaccaacactaatgttgatcttggcccacagaagcaggctgcgatagcttatatctcagcaaaatgcaaggatcaagggaaacgagatcagctctcaaagctcatcgagcgagcaaccaccttgagtgagatcaacaaagttaaaagacaggctcttggcctctagatcacccaatcacccccagtaatgagtacaacaataatcagaacctccctaaaccacatggccaaccaagcacaccatccacaccaccccttactatcctttgccagaaactccgccgcagctgatttattcaaaagaagccacttggtataacctagcaaccgcaagatagggtggggaaggtgctttgcctgcaagagggctccctcatcttcagacacttacccgccaacccaccagtgacacaatggcagacatggacactgtatatatcaatctgatggcagatgatccaacccaccaaaaagaactgctgtcctttcccctcattccagtgactggtcccgacgggaaaaaggaactccaacaccaggttcggactcaatccttgctcgcctcagacaagcaaactgagaggttcatcttcctcaacacttacgggtttatctatgacactacaccggacaagacaactttttccaccccagagcatatcaatcagcccaagagaacgatggtgagtgctgcaatgatgaccatcggcctggtccccgccaatatacccttgaacgaactaacagctactgtgtttggcctgaaggtgagagtgaggaagagtgcgagatatcgagaggtggtctggtatcagtgcaaccctgtaccagccctgctggcagccaccaggttcggtcgccaagggggtctcgaatcgagcactggagtcagtgtgaaggcccctgagaagatagattgtgagaaggattatacttactacccttatttcctatctgtgtgctacatcgctacttccaacctgttcaaggtaccaaaaatggttgctaatgcgaccaacagtcaattataccatctgaccatgcaggtcacatttgcctttccaaaaaacatccccccagctaaccagaaactcctgacacaagtggatgaaggattcgagggcactgtggactgccattttgggaacatgctgaaaaaggatcggaaagggaatatgaggacattgtcgcaggcggcagataaggtcagacggatgaacatccttgttggtatctttgacttgcatgggccgacactcttcctggagtataccgggaaactaacaaaagctctgctagggttcatgtctaccagccgaacagcaatcatccccatatctcagctcaatcctatgctgagtcaactcatgtggagtagtgatgcccagatagtaaaattaagagtggtcataactacatccaaacgcggcccatgcgggggtgagcaggagtatgtgctggatcccaaattcacagttaaaaaagaaaaagcccgactcaatcctttcaagaaggcagcccaatgatcaaatctgcaggatctcagaaatcagaccactctatactatccactgattaatagacacgtagctatacagttgatgaacctatgaagaatcaattagcaaaccgaatccttgctagggtggggaaggagttgattgggtgtctaaacaaaagcactcctttgcacctcctcgccacgaaacaaccataatgaggttatcacgcacaatcctggccctgattctaggcacacttaccggctatttaatggatgcccactccaccactgtgaacgagagaccaaagtctgaagggattaggggtgatcttataccaggcgcaggtatctttgtaactcaagtccgacaactacagatctaccaacagtctgggtatcatgaccttgtcatcaggttattacctcttctaccggcagaactcaatgattgtcaaagggaagttgtcacagagtacaacaatacggtatcacagctgttgcagcctatcaaaaccaacctggataccttattggctgatggtggtacaagggatgccgatatacagccgcggttcattggggcgataatagccacaggtgccctggcggtggctacggtagctgaggtgactgcagcccaagcactatcgcagtcgaaaacgaacgctcaaaatattctcaagttgagagatagtattcaggccaccaaccaggcagtttttgaaatttcacaaggacttgaggcaactgcaactgtgctatcaaaactgcaaactgagctcaatgagaacattatcccaagcctgaacaacttgtcctgtgctgctatggggaatcgccttggtgtatcactatcactctacttgaccttaatgaccaccctatttggggaccagatcacaaacccagtgctgacaccaatctcctatagcactctatcggcaatggcaggtggtcacattggcccggtgatgagtaagatattagccggatctgtcacaagtcagttgggggcagaacagttgattgctagcggcttaatacagtcacaagtagtgggttatgattcccaatatcaattattggttatcagggtcaatcttgtacggattcaagaggtccagaatacgagggtcgtatcactaagaacactagcggtcaatagggatggtggactttatagagcccaggtgcctcctgaggtagttgaacggtctggcattgcagagcgattttacgcagatgattgcgttcttactacaactgattacatttgctcatcgatccgatcttctcggcttaatccagagttagtcaagtgtctcagtggggcacttgattcatgcacatttgagagggaaagtgcattattgtcaacccctttctttgtatacaacaaggcagttgtcgcaaattgtaaagcagcaacatgtagatgtaataaaccgccgtctattattgcccaatactctgcatcggctctggtcaccatcaccactgacacctgcgccgaccttgaaattgagggttatcgcttcaacatacagactgaatccaactcatgggttgcaccaaacttcactgtctcgacttcacagattgtatcagttgatccaatagacatctcctctgacattgccaaaatcaacagttccatcgaggctgcaagagagcagctggaactaagcaaccagatcctctcccggattaacccacgaatcgtgaatgatgaatcactgatagctattatcgtgacaattgttgtgcttagtctcctcgtaatcggtctgattgttgttctcggtgtgatgtataagaatcttaagaaagtccaacgagctcaagctgccatgatgatgaagcaaatgagctcatcacagcctgtgaccactaaattagggacgcctttctaggaggataatcatattactctactcaatgatgagcaagacgtaccaattatcaatgattgtgtcacaaggccggttgggaatgcaccgaatctctcccctttctttttaattaaaaacatttgaagtgaggataagaggggggaagagtatggtagggtggggaaggtagccaatccctgcctattaggctgatcgtatcaaaagaacccaacagaagtctagatacagggcaacatggagggcagccgtgataatctaacagtggatgatgaattaaagacaacatggaggttagcttatagagttgtgtccctcctattgatggtgagcgctttgataatctctatagtaatcctgacaagagataacagccaaagcataatcacggcgatcaaccagtcatctgacgcagactctaagtggcaaacgggaatagaagggaaaatcacctccattatgactgatacgctcgataccagaaatgcagcccttctccacattccactccagctcaacacgcttgcggcgaacctattgtccgcccttggaggcaacacaggaattggccccggagatctggaacactgccgttaccctgttcatgacaccgcttacctgcatggagttaatcgattactcatcaaccagacagctgattatacagcagaaggccccctagatcatgtgaacttcataccagccccggttacgaccactggatgcacaaggataccatccttttctgtgtcatcgtccatttggtgctatacacacaacgtgattgaaaccggttgcaatgaccactcaggtagtaaccaatatatcagcatgggagtcattaagagagcaggcaacggcttaccttacttctcaacagttgtaagtaagtatctgactgatgggttgaataggaagagctgttctgtagctgccggatctgggcattgctacctcctttgcagcttagtgtcggagcctgaacctgatgactatgtatcacctgatcccacaccgatgaggttaggggtgctaacgtgggatgggtcttacactgaacaggtggtacccgaaagaatattcaagaacatatggagtgcaaactacccgggagtagggtcaggtgctatagtaggaaataaagtgttattcccattttacggcggagtgaggaatggatcgaccccggaggtgatgaataggggaagatactactacatccaggatccaaatgactattgccctgacccgctgcaagatcagatcttaagagcggaacaatcgtattacccaactcgattcggtaggaggatggtaatgcaaggggtcctagcatgtccagtatccaacaattcaacaatagcaagccaatgtcaatcttactattttaataactcattagggttcatcggggcagaatctagaatctattatctcaatggtaacatttatctttatcagagaagctcgagttggtggcctcacccccaaatctacctgcttgattctagaattgcaagtccgggtactcagaccattgactcaggtgtcaatctcaaaatgttaaatgtcactgtgattacacgaccatcatctggtttttgtaatagtcagtcacgatgccctaatgattgcttattcggggtctattcggatatctggcctcttagccttacctcagatagcatattcgcattcacaatgtatttacaggggaagacaacacgtattgacccggcttgggcgctattctccaatcatgcaattgggcatgaggctcgtctgtttaataaggaagttagtgctgcttattctaccaccacttgtttttcggacaccatccaaaatcaggtgtattgcctgagtatacttgaggtcagaagtgagctcttgggagcattcaaaatagtaccattcctctaccgcgtcttgtaggcatccattcagccaaaaaacttgagtgaccatgagattgacacctgatccccctcaaagacacctatctaaattactgttctagacccatgattaggtaccttcttaatcaatcatttggtttttaattaaaaatggaaaaatggacctagttccaagagagggctggaacccattagggtggggaaggattgctttgctccttgactcacactcacgtacactcgatcagacttctgttaaaaaggaaaccttctcaaactcgccccacgatgtccaatcaggcagctgagattatactacctagcttccatctagaatcacccttaatcgagaataagtgcttctattatatgcaattacttggtctcgtgttgccacatgatcactggagatggagggcattcgttaactttacagtggatcaggtgcaccttaaaaatcgtaatccccgcttaatggcccacatcgactacactaaagatagattgaggactcatggtgtcttaggtttccaccagactcagacaagtttgagccgttatcgtgttttgctccatcctgaaaccttaccttggctgtcagccatgggaggatgcatcaatcaggtgcctaaagcatggcggaacaccctgaaatcgatcgagcacagtgtaaagcaggaggcacctcaactaaagctactcatggagagaacctcattaaaattaactggggtaccttacttgttctctaattgcaatcccgggaaaaccaaagcaggaactatacctgtcctaagtgagatggcatcggaactcttgtcaaatcctatctcccaattccaatcaacatggggatgtgctgcttcggggtggcaccatgtagtcagtatcatgaggcttcagcaatatcaaagaaggacaggtaaggaggaaaaagcaatcactgaagttcagtatggcacagacacctgtctcattaacgcagactacaccgttgttttttccacacagaaccgtatcataacggtcttgcctttcgatgttgtcctcatgatgcaagacctgctcgaatcccgacggaatgtcctgttctgtgcccgctttatgtatcccagaagccaacttcatgagaggataagtacaatattagcccttggagaccaattggggaggaaagcaccccaagtcctgtatgattttgtagcaacccttgagtcatttgcatacgcagcggttcaacttcatgacaacaatcctacctacggtggggccttctttgaattcaacatccaagagttagaatcgattctgtcccctgcacttagtaaggatcaggtcaacttctacataagtcaagttgtctcagcgtacagtaaccttcctccatccgaatcggcagagctgctgtgcctgttacgcctgtggggtcatcccttgctaaacagccttgatgcagcaaagaaagtcagggagtctatgtgcgccgggaaggttctcgattacaacgccattcgacttgtcttgtctttttatcatacgttgctaatcaatgggtaccggaagaaacacaagggtcgctggccaaatgtgaatcaacattcacttctcaacccgatagtgaggcagctttattttgatcaggaggagatcccacactctgttgcccttgagcactatttggatgtttcaatgatagaatttgaaaaaacttttgaagtggaactatctgacagcctaagcatcttcctgaaggataagtcgatagctttggataagcaagaatggtatagtggttttgtctcagaagtgactccgaagcacctgcgaatgtcccgtcatgatcgcaagtctaccaataggctcctgttagccttcattaactcccctgaattcgatgttaaggaagagcttaaatacttgactacgggtgagtacgccactgacccaaatttcaatgtctcatactcacttaaagagaaggaggtaaagaaagaagggcgcattttcgcaaaaatgtcacaaaagatgagagcgtgccaggttatttgtgaagaattgctagcacatcatgtggctcctttgtttaaagagaatggtgttactcaatcagagctatccctgacaaaaaatttgttggctattagccaactgagttacaactcgatggccgctaaggttcgattgctgcggccaggggacaagttcactgctgcacactatatgaccacagacctaaaaaagtactgtcttaattggcggcaccagtcagtcaaactgttcgccagaagcctggatcgactgtttgggttagaccatgctttttcttggatacatgtccgtctcaccaacagcactatgtacgttgctgacccctttaatccaccagactcagatgcatgcacaaatttagacgacaataagaataccgggatctttattataagtgcacgaggtggtatagaaggcctccaacaaaagctatggactggcatatcaattgcaattgcccaagcggcagcggccctcgaaggcttacgaattgctgctactctgcagggggataaccaagttttggcgattacaaaggaattcatgaccccagtcccagaagatgtaatccatgagcagctatctgaggcgatgtctcgatacaaaaggactttcacatacctcaattatttaatgggacatcagttgaaggataaggaaaccatccaatctagtgatttctttgtttactccaaaagaatcttcttcaatggatcaatcttaagtcaatgcctcaagaacttcagtaaactcactactaatgccactacccttgctgagaatactgtggccggctgcagtgacatctcttcatgcattgcccgttgtgtggaaaacgggttgccaaaggatgccgcatacatccagaatataatcatgactcggcttcaactattgctagatcattactattcaatgcatggcggcataaactcagagttagagcagccaacgttaagtatctctgttcgaaacgcaacctacttaccatctcaactaggcggttacaatcatttaaatatgactcgactattctgccgcaatatcggcgacccgcttaccagttcttgggcagagtcaaaaagactaatggatgttggtctcctcagtcgtaagttcttggaggggatattatggagacccccgggaagtgggacgttttcaacactcatgcttgatccgttcgcacttaacattgattacctgaggccgccagagacaattatccgaaaacacacccaaaaagtcttattgcaagattgtccaaaccccctattagcaggtgtcgttgacccaaactacaaccaagaattagagctgttagctcagttcttgcttgatcgggaaaccgttattcccagggctgcccatgccatctttgagttgtctgtcttggggaggaaaaaacatatacaaggattggtagatactacaaaaacaattattcagtgctcattggaaagacagccattgtcctggaggaaagttgagaacattgttacctacaacgcgcagtatttcctcggggccacccaacaggctgacactaatgtctcagaagggcagtgggtgatgccaggtaacttcaagaagcttgtgtcccttgacgattgctcggtcacgttgtctaccgtatcacggcgcatatcgtgggccaatctactgaactggagagctatagacggtttggaaaccccggatgtgatagagagtatcgatggccgccttgtacaatcatccaatcaatgtggcctatgtaatcaagggttggggtcctactcctggttcttcttgccctctgggtgtgtgttcgaccgtccacaagattcccgggtggttccaaagatgccatatgtggggtccaaaacagatgagagacagactgcatcagtgcaagctatacaaggatccacttgtcacctcagggcggcattgaggcttgtatcactctacctatgggcctatggggattctgacatatcatggctagaagctgcgacactggctcaaacacggtgcaacgtttctcttgatgacttgcgaatcttgagccctctcccttcttcggcgaatttacaccacagattaaatgacggggtaacacaggttaaattcatgcccgccacatcgagccgagtgtcaaagttcgtccaaatttgcaatgacaaccagaatcttatccgtgacgatggaagtgttgattccaatatgatttatcaacaggttatgatattagggcttggggagattgaatgcttgttagctgacccaattgatacaaacccagaacaattgattcttcatctacactctgataattcttgctgtctccgggagatgccaacgaccggctttgtaccagctctaggactgaccccatgtttaactgtcccaaagcacaatccttacatatatgatgatagcccaatacctggtgatttggatcagaggctcattcagaccaaatttttcatgggttctgacaatttggataatcttgatatctaccaacagcgagctttactgagtaggtgtgtggcttatgatgttatccaatcgatctttgcttgtgatgcaccagtctctcagaagaatgacgcaatccttcacactgactatcatgagaattggatctcagagttccgatggggtgaccctcgtattatccaagtaacggcaggctacgagttaattctgttccttgcataccagctttattatctcagagtgagaggtgatcgtgcaatcctgtgttatgttgacaggatactcaataggatggtatcttccaatctaggcagtctcatccagacactctctcatccagagattaggaggagattctcgttgagtgatcaagggttccttgttgagagggaactagagccaagtaagcccttggttaaacaagcggttatgttcttgagggactcagtccgctgcgctctagctactatcaaggcaggaattgagcctgagatctcccgaggtggctgtactcaggatgagctaagctttactcttaagcacttactgtgtcggcgtctctgtgtaatcgctctcatgcattcagaggcaaagaacttggttaaggttagaaaccttcctgtagaagagaaaaccgccttactgtatcagatgttggtcactgaggccaatgctaggaaatcaggatctgctagcattatcataaacctagtatcggcaccccagtgggatattcatacaccagcattgtattttgtgtcaaagaaaatgttagggatgcttaagaggtcaaccacacccttggatataagtgacctctctgagagccagaatcccgcaccggcagagctgaatgatgttcctgatcacatggcagaagaatttccctgtttgtttagtagttataacgctacatatgaagacacaatcacttacaatccaatgactgaaaaactcgccttgcacttggacaatagttccaccccatccagagcacttggtcgtcactacatcctgcggcctcttgggctttactcatctgcatggtaccggtctgcagcactactagcatcaggggccctaaatgggttgcctgaggggtcaagcctgtatctaggagaagggtacgggaccaccatgactctgcttgagcccgttgtcaagtcttcaactgtttactaccacacattgtttgacccaacccggaatccttcacagcggaactataaaccagaaccacgggtattcacggattctatttggtacaaggatgatttcacacggccacctggtggtattatcaacctgtggggtgaagatatacgtcagagtgatatcacacagaaagacacggtcaacttcatactatctcagatcccgccaaagtcacttaagttgatacacgttgatattgaattctcaccagactccgatgtacggacactactttctggctattctcattgtgcattattggcctactggctattgcaacctggagggcgatttgcggttagggttttcttaagtgaccatgtcatagtaaacttggtcactgcaattctgtctgcttttgactctaatttggtgtgcattgcatcaggattgacacacaaggatgatggggcaggttatatttgcgcaaagaagcttgcaaatgttgaggcttcaaggattgaatactacctgaggatggtccatggttgtgttgactcattaaagatccctcatcaattaggaatcattaaatgggccgagggtgaggtgtcccaacttaccagaaaggcagatgatgaaataaattggcggttaggtgatccggttaccagatcatttgatccagtttctgagctaatcattgcacgaacaggggggtctgtattgatggaatacggggcttttactaacctcaggtgtgcgaacttggcagatacatacaaacttctggcttcaattgtagagaccaccttaatggaaataagggttgaacaagaccagttggaagataattcgaggaggcaaatccaaatagtccccgcttttaacacgagatctgggggaaggatccgtacactgattgagtgtgctcagctgcagattatagatgttatttgtgtaaacatagatcacctctttcctagacaccgacatgttcttgtcacgcaacttacctaccagtcggtgtgccttggggacttgattgaaggcccccaaattaagacgtatctgagggccagaaagtggatccaacgtcggggactcaatgagacagttaaccatatcatcactggacaagtgtcacggaataaagcaagggattttttcaagaggcgcctgaagttggttggcttttcactctgcggtggttggagctacctctcactttaactgttcaagttgttgattattatgaataatcggagtcggaatcgtaaatagtaagccacaaagtcgtgaataaacaatgattgcattagtatttaataaaaaatatgtcttttatttcgt

SEQ ID NO: 6 - DNA, 15054 bp, Avian paramyxovirus 4
acgaaaaagaagaataaaaggcagaagccttttaaaaggaaccctgggctgtcgtaggtgtgggaaggttgtattccgagcgcgcctccgaggcatctactctacacctatcacaatggctggtgtcttctcccaatatgagaggtttgtggacaatcaatcccaagtgtcaaggaaggatcatcggtccctggcagggggatgccttaaagtcaacattcctatgcttgtcactgcatctgaagatcccaccactcgttggcaactagcgtgtttatctttgaggctcttgatctccaactcatcaaccagtgctatccgccagggggcaatactgactctcatgtcactaccatcacaaaatatgagagcaacggcagctattgctggttccacaaatgcagctgttatcaacactatggaagtcttgagtgtcaatgactggaccccatccttcgaccctaggagcggtctctctgaagaggatgctcaggttttcagagacatggcaaaggacctgccccctcagttcacctccggatcaccctttacatcagcattggcggaggggtttaccccagaagacacccacgacctaatggaggccttgactagtgtgctgatacagatctggatcctggtggctaaggccatgaccaacattgatggctctggagaggccaatgagagacgtcttgcaaagtacatccagaagggacaactcaatcgccagtttgcaattggtaatcctgctcgtctgataatccaacagacgatcaaaagctccttaactgtccgcagattcttggtctctgaacttcgtgcatcacgaggtgcggtgaaagaaggatccccttactatgcagctgttggggacatccacgcttacatctttaacgcaggactgacaccattcttgactaccttaagatatgggatcggcaccaagtatgctgcagttgcactcagtgtgttcgctgcagacattgcaaaattaaagagcctacttaccctatatcaagacaagggtgtggaggctggatacatggcactccttgaagatccagactccatgcactttgcacctggaaacttcccacacatgtactcctacgcgatgggggtggcttcttaccatgaccccagcatgcgccagtaccaatatgctaggaggttcctcagccgacctttctacttgctagggagggacatggccgccaagaacacaggcacgctggatgagcaactggcaaaggaactgcaagtgtcagaaagagaccgcgccgcattgtccgctgcgattcagtcagcaatagaggggggagaatccgacgacttcccactgtcgggatccatgccggctctctccgacaatgcgcaaccagttaccccaagaacccaacagtcccagccctcccctccccaatcatcaagcatgtctcaatcagcacccaagaccccggactaccagcctgattttgaactgtaggctgcatcagtgcaccaacagcaggccaaagggaccaccctcctccccacacatcccacccaatcacccgctgagacccaatccaacaccccagcatccccctcatttaattaaaaactgaccaatagggtggggaaggagagctgttggctatcgccaagatcgtgcagcgatggattttaccgatattgatgctgtcaactcattaattgaatcatcatcagcaatcatagattccatacagcatggagggctgcaaccatcaggtactgttggcctatcgcaaatccccaaggggataaccagcgctttaaccaaggcctgggaggctgagacagcaactgctggctacggggacacccaacacaaatctgacagtccggaggatcatcaggccaacgacacagactcccccgaagacacaggcaccaaccagaccatccaggaagccaacatcgtcgaaacaccccaccccgaagttctatcggcagccaaagccagactcaagaggcccaaggcagggaaggacacccacgacaatccccctgcgcaacccgatccccttttaaaggggggccccctgagcccacaaccagcagcaccgtgggtgcaaaattcacccattcatggaggtcccggcaccgccgatccccgcccatcacaaactcaggatcattccctcaccggagagagatggcaatcgtcaccgataaagcaaccggagacattgaactggtggaatggtgcaacccggggtgcacagcaatccgaactgaaccaaccagactcgactgtgtatgcggatactgccccaccatctgcagcctctgcatgtatgacgactgatcaggtacaactattaatgaaggaggttgccgatatgaaatcactccttcaggcactagtgaggaacctagctgtcctgcctcaactaaggaacgaggttgcagcaatcaggacatcacaggctatgatagaggggacactcaattcaatcaagattctcgaccctgggaattatcaagaatcatcactgaacagttggttcaaaccacgccaagatcacgcggttgctgtgtccggaccagggaatccattgaccatgccaactccaatccaagacaacaccatattcctggatgaactggcaagacctcatcctagtttggtcaatccgtccccgcccactaccaacactaatgttgaccttggcccacagaagcaggctgcgatagcttatatctcagcaaaatgcaaggatcaagggagacgagatcagctctcaaagctcatcgagcgagcaaccaccttgagtgagatcaacaaagtcaaaagacaggcccttggcctctagaccactcgaccacccccagtaatgaacacaacaataatcagaacctccctaaaccacacggccaacccagcacaccatccacaccgcccaccactatcccccgccaaaaactccgctgcagccgatttattcaaaagaagccacttgatatgacttatcaaccgcaaggtagggtggggaaggtgctttgcctgcaagagggctccctcatcttcagacacgtacccgccaacccaccagtgacgcaatggcagacatggacactgtatatatcaatctgatggcagatgatccaacccaccaaaaagaactgctgtccttccctctcattccagtgactggtcccgacgggaaaaaggaactccaacaccaggttcggactcaatccttgctcgcctcagacaagcaaactgagaggttcatcttcctcaacacttacgggtttatctatgacactacaccggacaagacaactttttccaccccagagcatatcaatcagcccaagagaacgatggtgagtgctgcaatgatgaccatcggcctggtccccgccaatatacccttgaacgaactaacagctactgtgtttggcctgaaagtaagagtgaggaagagtgcgagatatcgagaggtggtctggtatcagtgcaaccctgtaccagccctgcttgcagccaccaggtttggtcgccaaggaggtctcgaatcgagcactggagtcagtgtgaaggcccccgagaagatagattgcgagaaggattatacttactacccttatttcctatctgtgtgctacatcgccacttctaacctgttcaaggtaccaaaaatggttgctaatgcgaccaacagtcaattataccacctgacgatgcaggtcacatttgcctttccaaaaaacattcccccagctaaccagaaactcctgacacaagtggatgaaggattcgagggcactgtggactgccattttgggaacatgctgaaaaaggatcggaaagggaatatgaggacattgtcgcaggcggcagataaggtccgacggatgaacatccttgttggtatctttgacttgcatgggccgacactcttcctggagtataccgggaaactaacgaaagctctgttagggttcatgtctaccagccgaacagcaatcatccccatatctcagctcaatcctatgctgagtcaactcatgtggagcagtgatgctcagatagtaaaattaagagtggtcataactacatccaaacgcggcccatgcgggggtgagcaggaatatgtgctggaccccaaattcacagttaaaaaagaaaaagcccgactcaaccctttcaagaaggcagcttaatgatcaaatctgcaggatctcaggaatcagaccactctatactatctactgatcaatagatatgtagctatacagttgatgaacctatgaagaatcaattagcaaaccgaatccttgctagggtggggaaggaattgattgggtgtctaaacaaaagcacttctttgcacctactcaccacaaaacaatcataatgaggttatcacgaacaatcctggccctgattctcggcgcacttaccggctatttaatggatgcccactccaccactgtgaatgagagaccaaagtctgaggggattaggggtgaccttataccaggtgcaggaatctttgtaactcaaatccggcaactacagatctaccaacaatctgggtatcatgaccttgtcatcaggttattacctcttttaccggcagaactcaatgattgccaaagggaagttgtcacagagtacaacaatacagtatcacagctgttgcagcctatcaaaactaacctggataccttattggctgatggtggcacaagggatgccgatatacagccgcggttcattggggcgataatagccacaggtgccctggcagtggctacggtagctgaggtgactgcagcccaagcactatctcagtcgaaaacgaacgctcaaaatattctcaagttgagagatagtattcaggccaccaaccaggcagtttttgaaatttcacaaggacttgaggcaactgcaactgtactatcaaaactgcaagctgagctcaatgagaacattatcccaagtctgaacaacttgtcctgtgctgccatggggaatcgccttggtgtatcactatcactctacttgaccctaatgactaccctatttggggaccagatcacaaacccagtgctgacaccaatctcctatagcactttatcggcaatggcaggtggtcacattggcccggtgatgagtaaaatattagccggatctgtcacaagtcagttgggggcagaacagttgattgctagcggcttaatacaatcacaggtagtaggttatgattcccaatatcaattattggttatcagggtcaaccttgtacggattcaagaggtccagaatacgagggtcgtatcactaagaacactagcggtcaatagggatggtggactttatagagcccaggtgcctcccgaggtagtcgaacggtctggcattgcagagcgattttatgcagatgattgtgttcttactacaactgattacatttgctcctcgatccgatcttctcggcttaatccagagttagtcaaatgtctcagtggggcacttgattcatgcacatttgagagggaaagtgcattattgtcaacccctttctttgtatacaacaaggcagttgtcgcaaattgtaaagcggcaacatgtagatgcaataaaccgccgtctattattgcccaatactctgcatcagctctggtcaccatcaccaccgacacctgcgccgaccttgaaattgagggctatcgcttcaatatacagactgaatccaactcatgggttgcaccaaacttcactgtctcgacttcacagattgtatcagttgatccaatagacatctcctctgacattgctaaaatcaacagttccatcgaggctgcaagagagcagctggaactaagcaaccagatcctttcccgaattaacccacgaattgtgaatgatgaatcattgatagctattatcgtgacaattgttgtgcttagtctcctcgtaatcggtctgattgttgttctcggtgtgatgtataagaatcttaaaaaagtccaacgagctcaagctgccatgatgatgcagcagatgagctcatcacagcccgtgaccactaaattagggacgcccttctaggataataatcatatcactctactcaatgatgagcaagacgtaccaatcatcaatgattgtgtcacaaggccggtagggaatgcaccgaatttctcccctttctttttaattaaaaacatttgtagtgaggatgagaaggggaaaatgtttggtagggtggggaaggtagccaattcctgcctattaggccgaccgtatcaaaagaactcaacagaagtccagatacaaggtaacatggagggcagccgtgataatcttacagtggatgatgaattaaagacaacgtggaggttagcttatagagttgtgtcccttctattgatggtgagcgctttgataatctctatagtaatcctgacgagagataacagccaaagcgtaatcacggcgatcaaccagtcatctgaagctgactccaagtggcaaacgggaatagaagggaaaatcacctccattatgactgatacgctcgataccaggaatgcagcccttctccacattccactccagctcaactcgcttgaggcgaacctattgtccgcccttgggggcaacacaggaattggccccggagatatagagcactgccgttaccctgttcatgacaccgcttacctgcatggagttaatcgattactcatcaaccagacagctgattatacagcagaaggccccctagatcatgtgaacttcattccagccccggttacgaccactggatgcacaaggataccatccttttccgtgtcatcgtccatttggtgctatacacacaacgtgattgaaaccggttgcaatgaccactcaggtagtaaccaatatatcagcatgggagtcattaagagagcgggcaacggcctaccttacttctcaacagttgtaagtaagtatctgactgatgggttgaataggaaaagctgttctgtagctgccggatctgggcattgctacctcctttgcagcttggtgtcggagcccgaatctgatgactatgtgtcacctgatcctacaccgatgaggttaggggtgctaacgtgggatgggtcttacactgagcaggtggtacccgaaagaatattcaagaacatatggagtgcaaactacccaggagtagggtcaggtgctatagtaggaaataaggtgttattcccattttacggcggagtgagtaatggatcgaccccggaggtgatgaataggggaagatattactacatccaggatccaaatgactattgccctgacccgctgcaagatcagatcttaagggcggaacaatcgtattacccaactcgattcggtaggaggatggtgatgcaaggggtcctagcatgtccagtatccaacaattcaacaatagcaagccaatgtcaatcttactattttaataactcattagggttcattggggcagaatctaggatctattacctcaatgataacatttatctttaccagagaagctcgagctggtggcctcacccccagatttacctgcttgattctaggattgcaagtccgggtactcagaacattgactcaggtgtcaatctcaagatgttaaatgtcactgtaattacacgaccatcatctggtttttgtaatagtcagtcacgatgccctaatgactgcttattcggggtctactcggatatctggcctcttagccttacctcagatagcatattcgcattcacaatgtatttacaggggaagacaacacgtattgacccggcttgggcgctattctccaatcatgcgattgggcatgaggctcgtctgtttaataagaaggttagtgctgcttattctaccaccacttgtttttcggacaccgtccaaaatcaggtgtattgcctgagtatacttgaggtcaggagtgagctcttgggagcattcaaaatagtaccattcctctatcgcgtcttgtaggcatccattcagccagaaaacttgagtgaccatgatattaacacctgatccccctcaaagacacctatctaaattactgttctagactcatgattaggtaccttcttaatcaatcatttggtttttaattaaaaatgaaaaaataggcctagttccaagagagggctggaacccattagggtggggaaggattgctttgctccttgactcacacacacgtacactcgatcagactcctgtttaaaaggaatccttctcaaactcgccccacgatgtccaatcaggcggctgagattatactacccaccttccatctagaatcacccttaatcgaaaataagtgcttctattatatgcaattacttggtctcgtgttgccacatgatcactggagatggagggcattcgttaactttacagtggatcaggtgcaccttaaaaatcgtaatccccgcttgatggcccacatcgactacactaaggatagattaaggactcatggtgtcttaggtttccaccagactcagacaagtttgagccgttatcgtgttttgctccatcctgaaaccttatcttggctatcagccatggggggatgcatcaatcaggttcctaaagcatggcggaacactctgaaatcgatcgagcacagtgtaaagcaggaggcacctcaactaaagctactcatggagagaacctcattaaaattaactggagtaccttacttgttctctaattgcaatcccgggaaaaccacagcaggtactatgcctgtcctaagtgagatggcatcggaactcttgtcgaatcctatctcccaattccaatcaacatgggggtgtgctgcttcggggtggcaccatgtagtcagtatcatgaggctccaacaataccaaagaaggacaggtaaagaagagaaagcgatcactgaagttcagtatggcacagacacctgtctcattaatgcagactacactgttgtgttttccacacagaaccgtatcataacagtcttgccttttgatgttgtcctcatgatgcaagacctgctcgaatcccgacggaatgtcctgttctgtgcccgctttatgtatcccagaagccaacttcatgagaggataagtacaatattagctcttggagaccaactggggagaaaagcaccccaagtcctgtatgatttcgtagcaacccttgagtcatttgcatacgcggctgttcaacttcatgacaacaatcctacctacggtggggccttctttgaattcaatatccaagagttagaatccattctgtcccctgcacttagtaaggatcaggtcaacttctacataaatcaagttgtctcagcgtacagtaaccttcccccatctgaatcggcagaattgctgtgcctgttacgcctgtggggtcaccccctgctaaacagccttgatgcagcaaagaaagtcagggagtctatgtgcgccgggaaggttctcgattacaacgccattcgacttgtcttgtctttttatcatacgttgctaatcaacggataccggaagaaacacaagggtcgctggccaaatgtgaatcaacattcactcctcaacccgatagtgaggcagctttattttgatcaggaggagatcccacactctgttgctcttgagcactatttggacgtctcaatggtagaatttgaaaaaacttttgaagtggaattatctgacagcctaagcatcttcctaaaggataagtcgatagctttggataagcaagagtggtacagtggttttgtctcagaagtgactccgaagcacctgcgaatgtcccgtcatgatcgcaagtctaccaataggctcctgttagccttcattaactcccctgaattcgatgttaaggaagagcttaaatacttgactacgggtgagtacgccactgacccaaatttcaatgtctcatactcacttaaagagaaggaagtaaagaaagaggggcgcattttcgcaaaaatgtcacaaaagatgagagcatgccaggttatttgtgaagaattgctagcacatcatgtggctcctttgtttaaagagaatggtgttactcaatcagagctatccctgacaaaaaatttgttggctattagccaactgagttacaactcgatggccgctaaggtgcgattgctgagaccaggggacaagttcactgctgcacactatatgaccacagacctaaaaaagtactgtcttaattggcggcaccagtcagtcaaactgttcgccagaagcctggatcgactgtttgggttagaccatgctttttcttggatacatgtccgcctcaccaacagcactatgtacgttgctgaccccttcaatccaccagactcagatgcatgcattaatttagacgacaataagaacactgggatttttattataagtgcacgaggtggtatagaaggcctccaacaaaaactatggactggcatatcaattgcaattgcccaagcggcagcggccctcgaaggcttacgaattgctgctactctgcagggggataaccaagttttggcgattacaaaggaattcatgaccccagtcccagaggatgtaatccatgagcagctatctgaggcgatgtctcgatacaaaaggactttcacatacctcaattatttaatgggacatcaattgaaggataaggaaaccatccaatccagtgatttctttgtctattccaaaagaatcttcttcaatggatcaatcttaagtcaatgcctcaagaacttcagtaaactcactactaatgccactacccttgctgagaatactgtggccggctgcagtgacatctcttcatgcattgcccgttgtgtggaaaacgggttgcctaaggatgccgcatatatccagaatataatcatgactcggcttcaattattgctagatcattactattcaatgcatggcggcataaactcagaattagagcagccaactttaagtatctctgttcgaaacgcaacctacttaccatctcaactaggcggttacaatcatctaaatatgacccgactattctgccgcaatatcggcgacccgcttaccagttcttgggcggagtcaaaaagactaatggatgttggtctcctcagtcgtaagttcttggaggggatattatggagacccccgggaagtgggacgttttcaacactcatgcttgacccgttcgcacttaacattgattacctgaggccgccagaaacaattatccgaaaacacacccaaaaagtcttgttgcaagattgcccaaaccccctattagcaggtgtcgttgacccaaactacaaccaagaattagagctgttagctcagttcttgcttgatcgggagaccgttattcccagggctgcccatgccatctttgagttgtctgtcttggggaggaaaaaacatatacaaggattggtggacactacaaaaacaattattcagtgctcattggaaagacagccattgtcctggaggaaagttgagaacattgttacctacaacgcgcagtatttcctcggggccacccaacaggctgatactaatgtctcagaagggcagtgggtgatgccaggtaacttcaagaagcttgtgtcccttgacgattgctcggtcacgttgtctactgtatcacggcgcatatcgtgggccaatctactgaactggagagctatagatggtttggaaaccccggatgtgatagagagtattgatggccgccttgtacaatcatcaaatcaatgtggcctatgtaatcaagggttggggtcctactcttggttcttcttgccctctgggtgtgtgttcgaccgtccacaagattcccgggtagttccaaagatgccatacgtggggtccaaaacagatgagagacagactgcatcagtgcaagctatacaaggatccacttgtcacctcagggcagcattgaggcttgtatcactctacttatgggcttatggagattctgacatatcatggctagaagctgcgacactggctcaaacacggtgcaatgtttctcttgatgacttgcgaatcttgagccctctcccttcttcggcgaatttacaccacagattaaatgacggggtaacacaggttaaattcatgcccgccacatcgagccgagtgtcaaagttcgtccaaatttgcaatgacaaccaaaatcttatccgtgatgatgggagtgttgattccaatatgatttatcaacaggttatgatattagggcttggggagattgaatgcttgttagctgacccaattgatacaaacccagaacaattgattcttcatctacactctgataattcttgctgtctccgggagatgccaacgactggctttgtacctgctctaggactgaccccatgtttaactgtcccaaagcacaatccttacatttatgatgatagcccaatacctggtgatttggatcagaggctcattcagaccaaatttttcatgggttctgacaatttggataatcttgatatctaccaacagcgagctttactgagcaggtgtgtggcttatgatgttatccaatcgatctttgcctgtgatgcaccagtctctcagaagaatgacgcaatccttcacactgactatcatgagaattggatctcagagttccgatggggtgaccctcgtattatccaagtaacggcaggctacgagttaattctgttccttgcataccagctttattatctcagagtgaggggtgaccgtgcaatcctgtgttatattgacaggatactcaataggatggtatcttccaatctaggcagtctcatccagacactctctcatccagagattaggaggagattctcattgagtgatcaagggttccttgttgaaagggaattagagccaggtaagcccttggttaagcaagcggttatgttcttgagggactcggtccgctgcgctttagcaactatcaaggcaggaattgagcctgagatctcccgaggtggctgtactcaggatgagctgagctttactcttaagcacttactatgccggcgtctctgtgtaatcgctctcatgcattcagaagcaaagaacttggttaaagtcagaaaccttcctgtagaggagaaaaccgccttactgtaccaaatgttggtcactgaggccaatgctaggaagtcaggatctgctagcattatcataaacctagtctcggcaccccagtgggacattcatacaccagcactgtattttgtgtcaaagaaaatgctagggatgcttaagaggtcaaccacacccttggatataagtgacctctccgagagccagaattccgcacctgcagagctgactgatgttcctggtcacatggcagaagagtttccctgtttgtttagtagttataacgccacatatgaagacacaattacttacaatccaacgactgaaaaactcgccttgcacttggacaacagttccaccccatccagagcacttggccgtcactacatcctgcggcctcttgggctttattcatccgcatggtaccggtctgcagcactactagcgtcaggggccttgaatgggttgcctgaggggtcaagcctgtatctaggagaagggtacgggaccaccatgactctgcttgagcccgttgtcaagtcttcaactgtttactaccatacattgtttgacccaacccggaatccttctcagcggaactataagccagaaccacgggtattcacggattctatttggtacaaggatgatttcacacggccacctggtggtattatcaacctgtggggtgaagatatacggcagagtgatatcacacagaaagacacggtcaacttcatactatctcagatcccgccaaaatcacttaagttgatacacgttgatattgaattctcaccagactccgatgtacggacactactatctggctattctcattgtgcactattagcctactggctattgcaacctggagggcgatttgcagttagggttttcttaagtgaccatatcatagtaaacttagtcactgcaattctgtctgcttttgactctaatttggtgtgcattgcatcaggattgacacacaaggatgatggggcaggttatatttgcgcaaagaagcttgcaaatgttgaggcttcaaggattgagcactacttgaggatggtccatggttgcgttgactcattaaagatccctcatcaattaggaatcattaaatgggccgagggtgaggtgtcccaacttaccagaaaggcggatgatgaaataaattggcggttaggcgatcctgttaccagatcatttgatccagtttctgagctaatcattgcacgaacaggggggtctgtattaatggaatacggggcttttactaacctcaggtgtgcgaacttggcagatacatacaagcttctggcttcaattgtagagaccaccctaatggaaataagggttgagcaagatcagttggaagataattcgaggagacaaatccaagtagtccccgctttcaacacgagatctgggggaaggatccgtacgctgattgagtgtgctcagctgcagattatagatgttatttgtgtaaacatagaccacctctttcctaaacaccgacatgttcttgtcacgcaacttacctaccagtcggtgtgccttggggacctgattgaaggcccccaaattaagacgtatctaagggccagaaagtggatccaacgtcagggactcaatgagacagttaaccatatcatcactggacaagtgtcacggaataaagcaagggattttttcaagaggcgcttgaagttggttgggttttcactctgcggtggttggagctacctctcactttagctgttcaggttgtcgattattatgaataatcggagtcggaatcgcaaataggaagccacaaagttgtggagaaacaatgattgcattagtatttaataaaaaatatgtcttttatttcgt

SEQ ID NO: 7 - DNA, 15054 bp, Avian paramyxovirus 4
acgaaaaagaagaataaaaggcagaagccttttaaaaggaaccctgggctgtcgtaggtgtgggaaggttgtattccgagtgcgccttcgaggcatctactctacacctatcacaatggctggtgtcttctcccagtatgagaggtttgtggacaatcaatcccaagtgtcaaggaaggatcatcgttccctggcagggggatgcctaaaagtcaacatccctatgcttgtcactgcatctgaagatcccaccactcgttggcaactagcatgtttatccttaaggctcttggtctccaactcatcaaccagtgctatccgccagggggcgatactgactctcatgtcactaccatcacaaaatatgagagcaacggcagctattgctggttccacaaatgcggctgttatcaacactatggaagtcttgagtgtcaacgactggaccccatccttcgaccccaggagcggtctctctgaagaggatgctcaggttttcagagacatggcaagggacctgccccctcagttcacctccgggtcaccctttacatcggcattggcggaggggtttaccccggaggacacccacgacctaatggaggccctgaccagtgtgctgatacagatctggatcctggtggctaaggccatgaccaacattgatggctctggggaagccaatgagagacgtcttgcaaagtacatccagaagggacagcttaatcgccagtttgcaattggtaatcctgctcgtctgataatccaacagacgatcaaaagctccttaactgtccgcaggttcttggtctctgagcttcgtgcatcacgaggtgcggtgaaagaaggatccccttactatgcggctgttggggatatccacgcttacatctttaacgcaggactgacaccattcttgactaccttaagatacgggataggcaccaaatatgctgctgttgcactcagtgtgttcgctgcagacattgcaaaattaaagagtctacttaccctataccaggacaagggtgtggaggccggatacatggcactcctcgaagatccagactctatgcactttgcgcctggaaacttcccacacatgtactcctacgcgatgggggtggcttcttaccatgaccccagcatgcgccagtaccaatatgctaggaggttcctcagccgtcctttctacttgctagggagggacatggctgccaagaacacaggcacgctggatgagcaactggcaaaggaactacaagtgtcagaaagagaccgtgccgcattgtccgctgcgattcaatcagcaatggaggggggagaatctgacgacttcccactatcgggatccatgccggctctctccgacaatgcgcaaccagttaccccaagaactcaacagtcccagctctcccctccccaatcatcaagcatgtctcaatcagcgcccaggaccccggactaccagcctgattttgaactgtaggctgcatccacgcaccaacagcaggccaaagaaaccacccccctcctcacacatcccacccaatcacccgccaagacccaatccaacaccccagcatccccctcatttaattaaaaactgaccaatagggtggggaaggagagttattggctattgccaagttcgtgcagcaatggattttaccgatattgatgctgtcaactcattaattgaatcatcatcagcaatcatagattccatacagcatggagggctgcaaccatcaggcactgtcggcctatcacaaatcccaaaggggataaccagcgccttaaccaaggcctgggaggccgaggcagcaactgctggcaacggggacacccaacacaaatctgacagtccggaagaccatcaggccaacgacgcagactcccccgaagacacaggcaccaaccagaccatccaagaagccaatatcgttgaaacaccccaccccgaagtgctatcggcagccaaagccagactcaagaggcccaagacagggagggacacccacgacaatccctctgcgcaacctgatcatcttttaaaggggggccccctgagcccacaaccagcggcaccgtgggtgaaagatccatccattcatggaggtcccggcaccgccgatccccgcccatcacaaactcaggatcattccctcaccggagagagatggcaatcgtcaccgacaaagcaaccggagacatcgaactggtggaatggtgcaacccggggtgcacagctatccgagctgaaccaaccagactcgactgtgtatgcggacactgccccaccatctgcagcctctgcatgtatgacgactgatcaggtacaactattaatgaaggaggttgccgacatgaaatcactccttcaggcactagtgaggaacctagctgtcctgcctcaactaaggaatgaggttgcagcaatcaggacatcacaggccatgatagaggggacactcaattcaatcaagattctcgaccctgggaattatcaagaatcatcactaaacagttggttcaaaccacgccaagatcacgcggttgttgtgtccggaccagggaatccattggccatgccaaccccgatccaagacaacaccatattcctagatgaactggcaagacctcatcctagtttggtcaatccgtccccgcccgctaccaacaccaatgctgatcttggcccacagaagcaggctgcgatagcttatatctcagcaaaatgcaaggatcaagggaaacgagaccagctctcaaagctcatcgagcgagcaaccaccctgagcgagatcaacaaagtcaaaagacaggcccttggcctctagaccactcgaccacccccagtgatgaatacaacaataatcagaacctccctaaaccacatggccaacccagcgcaccatccacaccacctattactacccttcgccagaaactccgccgcagccgatttattcaaaagaagccactcgatatgacttagcaaccgcaagatagggtggggaaggtgctttacctgcaagagggctccctcatcttcagacacgcacccgccaacccaccagtgacgcaatggcagacatggacactgtatatatcaatctgatggcagatgatccaacccaccaaaaagaactgctgtcctttcccctcattcccgtgactggtcctgacgggaaaaaggaactccaacaccaggtccggactcaatccttgctcgcctcagacaagcaaactgagaggttcatcttcctcaacacttacgggtttatctatgacactacaccggacaagacaactttttctaccccagagcatatcaatcaacccaagagaacgatggtgagtgctgcaatgatgaccatcggcctggtccccgccaatatacccttgaacgaactaacagctactgtgtttggcctgaaaataagagtgaggaagagtgcgagatatcgagaggtggtctggtaccagtgcaaccctgtaccagccctgcttgcagccacaaggtttggtcgccaaggaggtctcgaatcgagcactggagttagtgtaagggcccccgagaagatagactgcgagaaggattatacttactacccttatttcctatctgtgtgctacatcgccacttccaacctgttcaaggtaccaaaaatggtcgctaatgcgaccaacagtcaattataccacctgaccatgcagatcacatttgcctttccaaaaaacatccccccagctaaccagaaactcctgacactagtggatgaaggattcgagggcactgtggactgccattttgggaacatgctgaaaaaggatcggaaagggaacatgaggacactgtcgcaggcggcagacaaggtcagacggatgaacatccttgttggtatctttgacttgcatgggccaacactcttcctggagtacaccgggaagctaacaaaagctctgttagggttcatgtctaccagccgaacagcaatcatccccatatctcagctcaatcctatgctgagtcaactcatgtggagcagtgatgcccagatagtaaaattaagagtggtcataactacatccaaacgcggcccatgcgggggtgagcaggagtatgtgctggatcccaaattcactgttaaaaaagagaaagcccgactcaaccctttcaagaaggcagcccaatgatcaaatctacaagatctcaggaatcagaccactctatactatccactgatcaatagacatgtagctatacagttgatgaacctatgaagaatcagttagaaaaccgaatccttactagggtggggaaggagttgattgggtgtctaaacaaaaacattcctttacacctcctcgccacgaaacaaccataatgaggttatcacgcacaatcctgaccttgattctcggcacacttactgattatttaatgggtgctcactccaccaatgtaactgagagaccaaagtctgaggggattaggggtgatcttacaccaggcgcaggtatctttgtaactcaagtccgacaactacagatctaccaacagtctgggtatcatgaccttgtcatcagattattacctcttctaccggcagaactcaatgattgtcaaagggaagttgtcacagagtacaacaatacggtatcacagctgttgcagcctatcaaaaccaacctggataccttactggctggtggtggcacaagggatgccgatatacagccgcggttcattggggcaatcatagccacaggtgccctggcggtggctacggtagctgaggtgactgcagcccaagcactatctcagtcgaaaacaaacgctcaaaatattctcaagttgagggatagtattcaggccaccaaccaggcagttttcgaaatttcacaaggactcgaggcaactgcaactgtgctatcaaaactgcaaactgagctcaatgagaacattatcccaagcctgaacaacttgtcctgtgctgccatgggtaatcgccttggtgtatcactatcactctacttgaccttaatgaccaccctatttggggaccagatcacaaacccagtgctgacaccgatctcctatagcactctatcggcaatggcaggtggtcatattggcccggtaatgagtaaaatattagccggatctatcacaagtcagttgggggcggaacagttgattgctagcggcttaatacagtcacaggtagtaggttatgattcccaataccaattattggttatcagggtcaaccttgtacggattcaagaggtccagaatacgagagtcgtatcactaagaacactagcagtcaatagggacggtggactctatagagcccaggtgcctcccgaggtagttgaacggtctggcattgcagaacgattttatgcagatgattgtgttcttactacaaccgattacatttgctcatcgatccgatcttctcggcttaatccagagttagttagatgtctcagtggggcacttgattcatgcacatttgagagggaaagtgcattattgtcaacccctttctttgtatacaacaaggcagttgtcgcaaattgtaaagcagcaacatgtagatgtaataaaccgccgtctattattgcccaatactctgcatcagctctggtcaccatcaccaccgacacctgtgccgacctcgaaattgagggttatcgcttcaacatacagactgaatccaactcatgggttgcaccaaacttcactgtctcgacttcacagattgtatcagttgatcccatagacatctcttctgacattgccaaaatcaacagttccatcgaggctgcaagagagcagctggaactaagcaaccagatcctttcccggatcaacccacgaatcgtgaatgatgaatcactgatagctattatcgtgacaattgttgtgcttagtcccctcgtaatcggtctgattgttgttctcggtgtgatgtataagaatcttaggaaagtccaacgagctcaagctgccatgatgatgcagcaaatgagctcatcacagcctgtgaccactaaattagggacgcctttctaggagaacaaccatatcactccactcaatgatgagcaagacgtaccaatcatcaatgattgtgtcacaaggccggttgggaatgcatcgaatctctcccctttctttttaattaaaaacatttgaagtgaagatgagaggggggaagtgtatggtagggtggggaaggcagccaattcctgcccattaggccgaccgtatcaaaaggattcaatagaagtctaggtacagggtaacatggagggcagccgcgataatcttacagtggatgatgaattaaagacaacatggaggttagcttatagagttgtgtctcttctattgatggtgagcgctttgataatctctatagtaatcctgacgagagataacagccaaagcataatcacggcgatcaaccagtcatctgacgcagactctaagtggcaaacgggaatagaagggaaaatcacctccattatggctgatacgctcgataccaggaatgcagttcttctccacattccactccagctcaacactcttgaggcgaacctattgtctgcccttgggggcaacacaggaattggccccggagatctagagcactgccgttaccctgttcatgacaccgcttacctgcatggagttaatcgattactcatcaatcagacagctgattatacagcagaaggccccctagatcatgtgaacttcattccagccccggttacgactactggatgcacaaggataccatccttttccgtgtcatcgtccatttggtgctatacacataacgtgattgaaaccggttgcaatgaccactcaggtagtaatcaatatatcagcatgggagtcattaagagagcgggcaacggcctaccttacttctcaacagttgtaagtaagtatctgactgatgggttgaataggaaaagctgttctgtggctgccggatctgggcattgctacctcctttgcagcttagtgtcggagcccgaacctgatgactatgtgtcacctgatcctacaccgatgaggttaggggtgctaacgtgggatggatcttacactgaacaggtggtacccgaaagaatattcaggaacatatggagtgcaaactacccaggagtagggtcaggtgctatagtaggaaataaggtgttattcccattttacggcggagtgaggaatggatcgaccccggaggtgatgaataggggaaggtactactacatccaggatccaaatgactattgccctgacccgctgcaagatcagatcttaagggcggaacaatcgtattacccaactcgattcggtaggaggatgataatgcagggggtcctagcatgtccagtatccaacaattcaacaatagcaagccaatgtcaatcttactattttaataactcattagggttcattggagcagaatctagaatctattacctcaatagtaacatttacctttatcagaggagctcgagctggtggcctcacccccagatttacctgcttgattctaggattgcaagtccgggtactcagaacattgactcaggtgtcaatctcaagatgttaaacgtcactgtgattacacgaccatcatctggtttttgtaatagtcagtcacgatgccctaatgactgcttattcggggtctactcggatatctggcctcttagccttacctcggatagcatattcgcgttcactatgtatttacaggggaagacaacacgtattgacccggcttgggcgctattctccaatcatgcgattgggcatgaggctcgtctgtttaataaggaggttagtgctgcttattctaccaccacttgttttttggacaccatccaaaaccaggtgtattgcctgagtatacttgaggtcaggagtgagctcttgggagcattcaaaatagtaccattcctctatcgtgtcttgtaggcatccattcggccaaaaaacttgagtgactatgaggttaacacttgatcccccttaaagacacctatctaaattactgtcctagacccatgattaggtaccttttaaatcaatcatttggtttttaattaaaaatgaaaaaatgggcctagtttcaagagagggctggaacccactagggtggggaaggattgctttgctccttgactcacacccacgtatactcgatctcacttctgtaaagaagggatccttctcaaactcgccccacaatgtccaatcaggcagctgagattatactacccaccttccatctagaatcacccttaatcgagaataagtgcttttattatatgcaattacttggtctcgtgttgccacatgatcattggagatggagggcattcgttaactttacagtggatcaggtgcaccttaaaaatcgtaatccccgcttaatggcccatatcgaccacactaaagatagattaaggactcatggtgtcttaggtttccaccagactcagacaagtttgagccgttatcgtgttttgctccatcctgaaaccttaccttggctatcagccatgggaggatgcatcaatcaggttcctaaagcatggcggaatactctgaaatcgatcgagcatagtgtaaagcaggaggcacctcaactaaagctactcatggagagaacctcattaaaattaactggagtaccttacttgttctctaattgcaatcccgggaaaaccacagcaggaactatgcctgtcctaagtgagatggcatcggaactcttgtcaaatcctatctcccaattccaatcaacatgggggtgtgctgcttcggggtggcaccatgtagtcagtatcatgaggctccaacaatatcaaagaaggacaggtaaggaagagaaagcaatcaccgaagttcagtatggcacagacacttgtctcattaacgcagactataccgttgttttttccacacagaaccgtgttataacggtcttgcccttcgatgttgtcctcatgatgcaagacctactcgaatcccgacggaatgttctgttctgtgcccgctttatgtatcccagaagccaacttcatgagaggataagtgcaatattagcccttggagaccaactggggagaaaagcaccccaagtcctgtatgatttcgtggcgaccctcgagtcatttgcatacgcagctgttcaacttcatgacaacaatcctacctacggtggggccttctttgaattcaatatccaagagttagaatctattctgtcccctgcacttagtaaggatcaggtcaacttctacataggtcaagttgtctcagcgtacagtaaccttcctccatctgaatcggcagaattgttgtgcctgctacgcctgtggggtcatcccttgctaaacagccttgatgcagcaaagaaagtcagggagtctatgtgtgccgggaaggttctcgattacaacgccattcgactcgtcttgtctttttaccatacattgttaatcaatgggtaccgaaagaaacacaagggtcgctggccaaatgtgaatcaacattcactcctcaacccgatagtgaggcagctctattttgatcaggaagagatcccacactctgttgcccttgagcactatttggatgtctcaatgatagaatttgaaaaaacttttgaagtggaactatctgacagcctaagcatcttcctgaaggataagtcgatagctttggataagcaagaatggtacagtggttttgtctcagaagtgactccgaagcacctacgaatgtctcgtcatgatcgcaagtctaccaataggctcctgttagctttcattaactcccctgaattcgacgttaaggaggagcttaagtacttgactacgggtgagtacgccactgacccaaatttcaatgtctcatactcacttaaagagaaggaagtaaaaaaagaagggcgcatattcgcaaaaatgtcacaaaagatgagagcatgccaggttatttgtgaagaattgctagcacatcatgtggctcctttgtttaaagagaatggtgttactcaatcagagctatccctgacaaaaaatttgttggctattagccaactgagttacaactcgatggctgctaaggtgcgattgctgaggccaggggacaagttcactgctgcacactatatgaccacagacctaaagaagtactgtctcaattggcggcaccagtcagtcaaactgttcgccagaagcctggatcgactgtttggattagaccatgcgttttcttggatacatgtccgtctcaccaacagcactatgtacgttgctgaccccttcaatccaccagactcagaggcatgcacagatttagacgacaataagaacaccgggatttttattataagtgcaagaggtggtatagaaggcctccaacaaaaattatggactggcatatcgattgcaattgcccaagcggcagcggccctcgaaggcttacgaattgctgctactctgcagggggataaccaagttttggcgattacgaaggaattcatgaccccagtcccagaggatgtaatccatgagcagctatctgaggcgatgtctcgatacaaaaggactttcacatacctcaattatttaatggggcatcagttgaaggataaagaaaccatccaatccagtgacttctttgtttattccaaaagaatcttcttcaatggatcgatcttaagtcaatgcctcaaaaacttcagtaaactcactactaatgccactacccttgctgagaatactgtggccggctgcagtgacatctcttcatgcattgcccgttgtgtggaaaacgggttgcctaaggatgccgcatatatccagaatataatcatgactcggcttcaactattgctagatcattactattcaatgcatggcggcataaattcagaattagagcagccaactttaagtatctctgttcgaaacgcaacctacttaccatctcaactaggcggttacaatcatttgaatatgacccgactattctgccgcaatatcggcgacccgcttaccagttcttgggcggagtcaaaaagactaatggatgttggtctcctcagtcgtaagttcttagaggggatattatggagacccccgggaagtgggacgttttcaacactcatgcttgacccgttcgcacttaacattgattacctgaggccgccagagacaattatccgaaaacacacccaaaaagtcttgttgcaagattgcccaaatcccctattagcaggtgtcgttgacccgaactacaaccaagaattagagctgttagctcagttcttgcttgatcgggaaaccgttattcccagggctgcccatgccatcttcgagttatctgtcttgggaaggaaaaaacatatacaaggattggtagatactacaaagacaattattcagtgctcattggaaagacagccattgtcttggaggaaagttgagaacattgttacctacaacgcgcagtatttcctcggggccacccaacaggctgatactaatgtctcagaagggcagtgggtgatgccaggtaaccttaagaagcttgtgtccctcgacgattgctcggtcacgctgtctactgtatcacggcgcatatcatgggccaatctactgaactggagagctatagatggtctggaaaccccggatgtgatagagagtattgatggtcgccttgtacaatcatccaatcaatgtggcctatgtaatcaagggttgggatcctactcctggtttttcttgccctctgggtgtgtgttcgaccgtccacaagattctcgggtagttccaaagatgccatacgtggggtccaaaacagatgagagacagactgcatcagtgcaagctatacaaggatccacttgtcacctcagggcagcattgaggcttgtatcactctacctatgggcctatggagattctgacatatcatggctagaagctgcaacgctggctcaaacacggtgcaatgtctctctcgatgatttgcgaatcttgagccctcttccttcttcggcgaatttacaccacagattaaatgacggggtaacacaggttaaattcatgcccgccacatctagccgagtgtcaaagttcgtccaaatttgcaatgacaaccagaatcttatccgtgatgatgggagtgttgattccaatatgatttatcaacaggttatgatattagggcttggagagattgaatgcttgttagctgacccaattgatacaaacccagaacaattgattcttcatctacactctgataattcttgctgtctccgggagatgccaacgaccggctttgtacctgctctaggactaaccccatgtttaactgtcccaaagcataatccttacatttatgacgatagcccaatacccggtgatttggatcagaggctcattcagaccaaatttttcatggggtctgacaatttggataatcttgatatctaccagcagcgagctttactgagtaggtgtgtagcttatgatgtcatccaatcgatctttgcctgtgatgcaccagtctctcagaagaatgacgcaatccttcacactgattaccatgagaattggatctcagagttccgatggggtgaccctcgtattatccaagtaacggcaggctatgagttaattctgttccttgcataccagctttattatctcagagtgaggggtgaccgtgcaatcctgtgctatatcgacaggatactcaataggatggtatcttccaatctaggtagtctcatccagacactctctcatccagagattaggaggagattctcgttgagtgatcaagggtttcttgttgaaagagaactagagccaggtaagcccttggttaaacaagcggttatgttcttaagggactcggtccgctgcgctttagcaactatcaaggcaggaattgagcctgaaatctcccgaggtggttgtactcaggatgagctgagctttactcttaagcacttactatgtcggcgtctctgtgtaatcgctctcatgcattcagaagcaaagaacttggttaaagttagaaaccttcctgtagaagagaaaaccgccttattgtaccagatgttggtcactgaggccaatgctaggaaatcagggtctgccagcattatcataaacctagtctcggcaccccagtgggacattcatacaccagcattgtattttgtgtcaaagaaaatgctagggatgcttaagaggtcaaccacacccttggatataagtgacctctctgagaaccagaaccccgcacctgcagagcttagtgatgctcctggtcacatggcagaagaattcccctgtttgtttagtagttataacgctacatatgaagacacaatcacttacaatccaatgactgaaaaactcgccttgcatttggacaacagttccaccccatccagagcacttggtcgtcactacatcctgcggcctcttgggctttactcatccgcatggtaccggtctgcggcactactagcgtcaggggccctaaatgggttgcctgaggggtcgagcctgtatttaggagaagggtacgggaccaccatgactctgcttgagcccgttgtcaagtcttcaactgtttactaccatacattgtttgacccaacccggaacccttcacagcggaactataaaccagaaccacgggtattcacggattctatttggtacaaggatgatttcacacggccacccggtggtattatcaacctgtggggtgaagatatacgtcagagtgatatcacacagaaagacacggtcaacttcatactatctcagatcccgccaaaatcacttaagttgatacacgttgatattgagttctcaccagactccgatgtacggacactactatccggctattctcattgtgcactattggcctactggctattgcaacctggagggcgattcgcagttagggttttcttaagtgaccatatcatagttaacttggtcactgcgatcctgtctgcttttgactccaatttggtgtgcattgcgtcaggattgacacacaaggatgatggggcaggttatatttgcgcgaaaaagcttgcaaatgttgaggcttcaagaattgagtactacttgaggatggtccatggttgtgttgactcattaaagatccctcatcaattaggaatcattaaatgggccgagggtgaggtgtcccagcttaccagaaaggcggatgatgaaataaattggcggttaggtgatccagttaccagatcatttgatccagtttctgagctaataattgcacgaacaggggggtctgtattaatggaatacggggcttttactaacctcaggtgtgcgaacttggtagatacatacaaacttctggcttcaattgtagagaccaccctaatggaaataagggttgagcaagatcagttggaagatagttcgaggagacaaatccaagtaatccccgctttcaacacaagatctgggggaaggatccgtacactgattgagtgtgctcagctgcagattatagatgttatttgtgtaaacatagatcacctctttcctaaacaccgacatgttcttgtcacacaacttacctaccagtcggtgtgccttggggatttgattgaaggtccccaaattaagacgtatctaagggccagaaagtggatccaacgtcggggactcaatgagacagttaaccatatcatcactggacaagtgtcacggaataaagcaagggatttttttaagaggcgcctgaagttggttggcttttcactctgcggaggttggagctacctctcactttagctgttcaggttgctgatcatcatgaacaatcggagtcggaatcgtaaacagaaagtcacaaaattgtggataaacaatgattgcattagtatttaataaaaaatatgtcttttatttcgt

SEQ ID NO: 8 - DNA, 15048 bp, Avian paramyxovirus 4
acgaaaaagaagaataaaaggcagaagccttttaaaaggaaccctgggctgtcgtaggtgtgggaaggttgtattccgagtgcgcctccgaggcatctactctacacctatcacaatggctggtgtcttttcccagtatgagaggtttgtggacaatcaatctcaggtgtcaaggaaggatcatcggtccttagcaggagggtgccttaaagtgaacatccctatgcttgtcactgcatccgaagaccccaccacgcgttggcaactagcatgcttatctctgaggctcttgatttccaattcatcaaccagtgctatccgccagggagcaatactgaccctcatgtcattgccatcgcaaaacatgagagcaacagcagctattgctgggtccacgaatgcggctgttatcaacactatggaagtcttaagtgtcaatgactggaccccatcttttgacccaagaagtggtctatctgaggaggacgctcaggtgttcagagacatggcaagagatctgcctcctcagttcacttctggatcaccctttacatcagcattggcggaggggtttactcccgaggacactcatgacctgatggaggcactgactagtgtactgatacagatctggattctggtggccaaggccatgaccaatattgatggatctggggaggctaacgaaagacgccttgcaaaatacatccaaaagggacagctcaatcgtcagtttgcaattggcaatcctgcccgtctgataatccaacagacaatcaaaagctcattaactgtccgcaggttcttggtctctgagctccgcgcatcacgtggtgcagtaaaggagggttccccttactatgcagccgttggggatatccacgcttacatcttcaatgcaggattgacaccattcttgaccaccctgagatatggcattggcaccaagtacgccgctgtcgcactcagtgtgtttgctgcagacattgcaaaattgaagagtctactcaccctgtatcaagacaaaggtgtagaagctggatacatggcactccttgaagatccagattccatgcactttgcacctggaaacttcccacacatgtattcctatgcgatgggagtggcctcctatcacgaccctagcatgcgccaataccagtatgccaggaggtttctcagtcgtcccttctacctgctaggaagagacatggctgctaagaacacaggaactctggatgagcagctggcgaaagaactgcaagtgtcagagagggaccgcgctgcactgtctgccgcgattcaatcagcaatggaggggggagagtcagatgacttcccattgtcaggatccatgccggccctctctgagagcacacaaccggtcacccccaggactcaacagtcccagctctctcctcctcaatcatcaaacatgtcccaatcggcgcctaggaccccggactatcaacccgactttgagctgtagactatatccacacaccgacaatagctccagaagacccccttcccccccatacaccccacccggtcatccacaaagacccagtccaacatcccagcactattcccttttaattaaaaactggccgacagggtggggaaggaggactgttagctgccaccaacggtgtgcagcaatggattttacagacattgacgctgtcaactcactgattgagtcatcatcggcaattatagactccatacagcatggagggctgcaaccagcaggcactgttggcttatctcaaattccaaaagggataaccagtgcactgaataaagcctgggaagctgaggcggcaactgccggcagtggagacacccaacacaaacccgatgacccagaggaccaccaggctagggacacggagtccctggaagacacaggcaacgacccggccacacaggggactaacattgttgagacaccccacccagaagtactgtcagcagccaaagctagactcaagagacccaaagcagggaaagacacccatggcaatccccccactcaacccgatcactttttaaaggggggcctcccgagtccacaaccgacagcaccgcggatgcaaagtccacccaaccatggaagctccagcaccgccgatccccgccaatcacaaactcaggatcattcccccaccggagagaaatggcaattgtcaccgacaaagcaaccggagacatcgaactggtggagtggtgcaacccagggtgtacagcagtccgaattgaaccagccagacttgactgtgtatgcggacactgccccaccatctgcagtctctgcatgtatgacgactgatcaggtacagttgttgatgaaggaggttgctgacataaaatcactcctccaggcactagtaaggaatctagctgtcttgccccaactaaggaatgaggttgcagcaatcagaacatcacaggccatgatagaggggacactcaattcaattaagattcttgatcctggaaattatcaggaatcatcactaaacagttggttcaaacctcgccaggaacacactgttattgtgtcaggaccagggaatccactggccatgccgactccagttcaggacagtaccatattcttagatgagctagcaagacctcatcctaatttggtcaatccgtctccgcccgtcaccagcaccaatgttgaccttggcccacagaagcaggctgcaatagcctacgtttccgccaagtgcaaggacccagggaaacgggaccagctttcaaggcttattgaacgggcggctaccttgagtgagatcaacaaggttaaaagacaggctctcgggctctaaattaatcaaccacccgttgcaacgatcgagacaacaataaaaatccccctgaatcacatgaccaaatctgcataccactcacatcatccgcctatacccctcaccataaataccaccttagccgatttatttaaaagaaatcattcatcacaacctggtaatcataaactagggtggggaaggtctcttgtctgcaggaaggctcctctgtctccaggcacgcacccgtcaacccaccaataacacaatggcggacatggacacgatatacatcaacttgatggcagatgatccaacccatcaaaaagaattgctgtcattccctctgattccagtgactggacctgatgggaagaaagtgctccaacaccagatccggacccaatccttgctcacctcagacaaacaaacggagaggttcatctttctcaacacttacgggttcatctatgacacaaccccggacaagacaactttttccacccctgagcatatcaatcagcctaagaggacaatggtgagtgctgcgatgatgactattggtctggttcctgctacaatacccctgaatgaattgacggccactgtgtttaaccttaaagtaagagtgaggaaaagtgcgaggtatcgagaagtggtttggtaccagtgcaaccccgtaccagctctgctcgcagccaccagatttggccgccaagggggtcttgagtcgagcaccggagtcagtgtaaaggcacctgagaagattgattgtgagaaagattatacttactacccttatttcctatctgtgtgctacatcgccacttccaacctctttaaggtaccgaagatggttgccaatgcaaccaacagtcaattgtatcacctaaccatgcaggtcacatttgcatttccgaaaaacattcccccagccaatcagaaactcctgacacaggtagatgaaggatttgagggtaccgtggattgccattttgggaacatgctaaaaaaggataggaaagggaacatgaggactttgtctcaagcagcagataaggtcagaagaatgaatatccttgtgggaatatttgacttgcacggacctacactattcctggaatatactgggaaattgacaaaagccctgttggggttcatgtccaccagccgaacagcaatcatccccatatcacaactcaatcctatgctgagtcaactcatgtggagcagtgacgcccagatagtaaagttacgggtggtcatcactacatctaaacgtggcccgtgtgggggcgagcaggaatatgtgctggatcctaaattcacagttaagaaagaaaaggctcgactcaatccattcaagaaggcagcctaataattaaacctacaagatcccaagaattaaacagctctatacaattcataggttgatagaaatgccactacacagctaatgattttccagaaaatcacttagaaaaccaaatccttattagggtggggaagtagttgattgggtgtctaaacaaaagtgcttctttgcaactccccaccccgaagcaatcacaatgagaccattaaacacgcttttgaccgtgattcttatcatactcatcagctatttggtgattgttcattctagtgatgcggttgagaggccaaggactgagggaattaggggcgacctcattccaggtgcgggtatcttcgtgactcaagtccgacaactgcaaatctatcagcagtcagggtaccacgaccttgtcataagattattaccccttttaccaacggaactcaatgattgccaaaaagaagtagtcacagaatacaataatacagtatcacaattgttgcagcctatcaaaaccaacttggataccctattagcagatggtaatacgagggaagcggatatacagccgcggtttattggagcaataatagccacaggtgccttggcggtagcaacagtggcagaagtaactgcagctcaggcactctcccagtccaaaacaaatgctcaaaatattctcaagctaagagatagtatccaggccaccaaccaagcggtctttgaaatttcacaagggcttgaggcaactgcaactgtgctatcgaaactacagacagagctcaatgagaatattatcccaagcctgaacaatttatcctgtgctgccatggggaatcgtcttggtgtatcactctcactctatttaactctaatgactaccctctttggggaccaaattacgaacccagtgctgacaccaatttcttacagcacactatcggcaatggcaggtggtcatattggcccagtgatgagtaaaatattagccggatcggtcacgagccagttgggggcagaacaattgattgctagtggcttaatacaatcacaggtggtaggctatgattcccagtatcaattattggtaatcagggttaaccttgttcggattcaggaagtccagaataccagggttgtatcattaagaacgctagctgtcaatagagatggtggactttatagagcccaagttccacctgaggtagtcgaacgatccggcattgcagagcggttttacgcagatgattgtgttctcaccacgaccgactatatttgctcatcaatcagatcctctcggcttaatccagaattagtcaagtgtctcagtggggcacttgattcatgtacattcgagagggagagtgccctgttatcaactcctttctttgtgtacaataaggctgtcgtagcaaattgcaaagcggcaacatgcagatgcaacaaaccaccgtcaattattgctcaatattctgcatcagctctagtaaccatcaccactgacacctgtgccgatctcgaaattgagggttaccgtttcaacatacagactgaatctaactcgtgggttgcacctaactttactgtctcaacctcacagatagtgtcagttgatccaatagacatatcctctgacatcgcaaaaatcaacaattcgattgaggccgcacgagagcagctagaactgagcaaccagatcctatcccggattaacccccgaatcgtgaatgacgaatcactgatagctattatcgtgacaattgttgtgcttagtctccttgtagtcggtcttatcattgttctcggcgtgatgtataaaaatctcaagaaggtccaacgagctcaggctgctatgatgatgcagcaaatgagttcatcgcagcctgtaaccacaaaactggggacacccttctaggtgaataaatgcatcacctctttccttgatgagcgagatgtcttaatcattgataattatgccgtaaggctggtagggaatgtgctgaatctctcctcttcctttttaattaaaaacggttgaactgagggggagaatgtgcatggtagggtggggaaggtgtctgattcctacctatcgggccaactgtaccagtagaagctaacaggaattctaatgcagagtgacatggagggcagtcgtgataacctcacagtggatgatgagttaaagacaacatggaggttagcttacagagttgtatctctcctattaatggtgagtgctttgataatttctatagtaatcttgacgagggataacagccaaagcataatcacggcaatcaaccagtcatatgatgcagactcaaagtggcaaacagggatagaggggaaaatcacctctatcatgactgatacgcttgatactaggaatgcagctctcctccacattccactccaacttaatacacttgaagcaaacctattatcagccctcggtggcaacacaggaatcggccccggggatctagagcattgccgttatccagttcatgattctgcttacctgcatggagtcaaccgattacttatcaatcaaacggctgattatacagcagagggtccactagatcatgtgaacttcataccggcaccagttacgaccactggatgcactaggataccatctttttccgtgtcctcatccatttggtgttatactcacaatgtgattgaaactggttttaatgatcactcaggcagcaatcagtatattagcatgggggtgattaagagggctggcaacggcttgccttatttctcaaccgttgtgagtaagtatctgaccgacggattgaataggaaaagttgttctgtggctgctgggtctgggcattgctatcttctctgcagcctagtatcagagcccgagcctgacgactatgtatcaccagaccccacaccgatgaggttaggggttctgacatgggatgggtcctatactgaacaggtggtgcctgaaaggatattcaaaaacatatggagtgcaaattaccctggggtgggatcaggtgctattgtgggaaataaggtgttgttcccattttacggaggagtgaggaatgggtcgacacctgaggttatgaataggggaaggtattactacattcaagatcctaatgattattgtcctgatccactgcaagaccaaatcttaagggcagaacaatcatattatcctacacggtttggtaggaggatggtgatgcagggtgtcttagcgtgcccagtgtccaacaactcaacaattgccagccaatgccagtcctactatttcaacaactcattagggttcattggggcggaatctaggatttattacctaaatgggaacctctacctttaccaaagaagctcgagctggtggccccacccccagatttatctgcttgaccccagaattgcaagcccgggcactcagaacatcgactcaggcattaatctcaagatgttgaatgttaccgttattacacgaccgtcatctggtttttgtaatagtcagtcaagatgccctaatgactgcttattcggggtctattcagacgtctggcctcttagcctaacctcagatagtatattcgcattcacgatgtatttacaagggaagacaacacgtattgacccggcgtgggcactgttctccaatcacgcaattgggcatgaagctcgtctattcaacaaggaggtcagtgctgcttactccactaccacttgcttttcggacaccatccaaaaccaggtgtattgcctgagtatacttgaagttagaagtgagcttttggggccattcaagatagtaccattcctctaccgtgtcctataggtgcctgctcgatcgagaactccaaataatcgtggaattagtacttaatcttccctatggatatctgccttaattactgtcctaggtctctggattagcgccctttaaaccagttttttgatttttaattaaaaatagaagattagacctggactcggggagggagaagaacctattagggtggggaaggattactttactccatgactcacaatcgcacacacctgacctcatttccactgagaaggaaccctcctcaaatttgatttgcaatgtccaatcaagcagctgagattatactccctacctttcacctagagtcacccttaatcgagaacaaatgcttctactatatgcaattacttggtcttatgttgccgcatgatcattggagatggagggcatttgtcaactttacagtggatcaagcacaccttagaaaccgtaatcctcgcttgatggcccacatcgaccacactaaggataaactaagggctcatggtgtcttaggtttccatcagacccaaacaggtgagagccgtttccgtgtcttgcttcacccggaaaccttaccatggctatcagcaatgggaggatgcataaaccaagtccccaaagcatggcggaacactctgaagtccatcgagcacagtgtgaagcaggaggcaacacaactacaatcgcttatgaaaaaaacctcattgaaattaacaggagtaccctacttattttccaactgtaatcccgggaaaaccacaacaggcactatgcctgtattaagcgagatggcatcagagctcctatcaaatcccatctcccaattccaatcaacatgggggtgtgctgcttcagggtggcaccatattgttagcatcatgaggcttcaacagtatcaaagaaggacaggtaaagaggagaaggcgatcactgaggttcattttggttcagacacctgtctcattaatgcagactacaccgttatcttttccttacagagccgtgtaataacagttttaccttttgacgttgtcctcatgatgcaagacctgctcgaatctcgacgaaatgtcctgttctgtgcccgctttatgtaccccagaagccaattgcatgagaggataagcatgatactagctctcggagatcaacttgggaaaaaggcaccccaagttctatatgactttgttgcaacccttgaatcatttgcatacgcagctgtccaacttcatgacaataaccctatctacggtgggactttctttgaattcaatatccaagaattagaatctatcttgtctcctgcgcttagcaaggaccaggtcaacttctacattagtcaggttgtctcagcatacagtaacctccccccatctgaatcggcagaattgctatgcctgttacgcctatggggtcaccctttactaaatagcctcgatgcagcaaagaaagtcagagaatcaatgtgtgccgggaaggttcttgactacaatgccattcgattagtcttgtctttttaccatacattattgatcaatggatatcggaagaaacacaagggacgctggccaaatgtgaatcaacattcactactcaacccaatagtgaggcagctttactttgatcaagaagagatcccacattctgtcgccctcgaacattacttagacatctcaatgatagaatttgagaaaacttttgaggttgaactatctgacagcctaagcatctttttgaaagacaagtcgattgccttggacaaacaagagtggtacagcggttttgtttcagaagtgaccccaaagcacttgcggatgtctcgtcatgaccgcaagtccaccaacaggctcctgctggcctttatcaactcccctgaattcgatgttaaagaagagctaaaatacttgactacaggtgagtatgctactgatccaaatttcaacgtttcttactcacttaaagagaaggaagtaaagaaagaaggacgaatctttgcaaaaatgtcacaaaagatgagagcgtgccaggttatttgtgaagagttgctagcacatcatgtagcccctttgtttaaagagaatggtgtcacacagtcggaactatctctgacaaaaaatctgctagctatcagtcagttgagttataactcaatggctgctaaggtgcggttgctgagaccaggggacaaattcactgccgcacactatatgaccacagacctgaaaaagtactgccttaattggcgtcaccagtcagtcaaactgtttgccagaagcctagatcgactgttcgggctagatcatgctttttcttggatacatgtccgcctcaccaacagcaccatgtatgtggctgatccattcaatccaccagactcagatgcatgcccaaacttagacgacaacaaaaacacgggaattttcatcataagtgcacgaggtgggatagaaggcctccaacaaaaactgtggaccggcatatcaatcgcaatcgcgcaagcagctgcagccctcgaaggcttgagaattgctgctactttgcagggggacaaccaggttctagcgatcacgaaggaatttgtaaccccagtcccggaaggtgtcctccatgagcaattatctgaggcgatgtcccgatataaaaagactttcacataccttaattacttaatggggcatcaactgaaagataaagagacaatccaatccagtgatttctttgtttactctaaaaggatattctttaatgggtccattctgagtcaatgtctcaaaaacttcagtaagctcaccactaatgccaccacccttgccgagaacactgtagccggctgcagtgacatctcatcatgcatcgctcgttgtgtagaaaacgggttgccaaaggatgctgcatacatccagaacatagtcatgactcgacttcaactgttgctagatcactactattccatgcatggtggcataaactcagaattagaacagccgaccctaagtatttctgttcggaatgcaacctatttaccatctcagttgggcggttacaatcatctaaatatgacccgactattttgccgcaacatcggtgacccgctcactagttcctgggcagaagcaaagagactaatggaagttggcctgctcaatcgtaaattcctggagggaatattgtggcgacctccgggaagtgggacattctcaacacttatgcttgacccgtttgcgctgaacattgattacctcagaccaccagagacaataatccgaaagcatacccagaaggtcttgctgcaagattgccctaatcccctattagccggtgtggttgatccgaactacaaccaggaactggaactattagcgcagttcttgctcgaccgagagaccgttattcccagggcagctcatgctatctttgagctgtctgtcttggggaggaaaaaacatatacaagggttggtggacactacaaaaacgattatccagtgttcgctggaaagacaaccattgtcctggaggaaagttgagaacattatcacctataatgcgcagtatttccttggagccactcagcagattgatacagattcccctgaaaagcagtgggtgatgccaagcaacttcaagaagctcgtgtctcttgacgattgttcagtcacattgtctactgtttcccggcgtatatcttgggccaacctacttaattggagggcaatagatggcttggaaaccccagatgtgatagaaagtattgatgggcgccttgtgcaatcatccaatcagtgtggcctatgtaatcaaggattaagttcctactcctggttcttcctcccctccggatgtgtgtttgatcgtccacaagactccagggtagtaccgaaaatgccgtatgtgggatccaagacagatgagaggcagactgcgtcggtacaagctatacagggatccacatgtcaccttagagcagcattgagacttgtatcactctacctttgggcttatggggattctgatatatcatggctggaagccgcgacactagcccaaacacggtgcaatatttcccttgatgatctgcgaatcctgagccctctaccttcctcggcaaatttacaccacagattaaatgacggggtaacacaagtgaaattcatgcctgctacatcaagccgagtatcaaagtttgtccagatttgcaatgacaaccagaatcttatccgtgatgatgggagtgtggattccaatatgatttatcagcaagtcatgatattaggacttggggaatttgagtgcttgttggccgacccaatcgatactaacccagagcaattgattcttcatctacactctgacaattcttgctgcctccgggagatgccaacaaccggctttgtgcctgctttgggattaaccccatgcttaactgtaccaaagcaaaatccatatatttatgacgagagtccaatacctggtgacctggatcaacggctcatccaaacaaagtttttcatgggttctgataatctagacaaccttgatatctatcagcaacgagcgttactaagtcggtgtgtggcttatgatgttatccaatcagtatttgcttgtgatgcaccagtttctcagaagaatgatgcaatcctccatactgactatcatgagaattggatctcagagttccgatggggtgaccctcggataattcaagtgacagcaggttatgaattgatcttgtttcttgcttaccagctttattaccttagagtgaggggtgaccgtgcaatcctgtgctatattgataggatactgaataggatggtgtcatcaaatctaggcagccttatccagacactctcccatccggagattaggaggaggttttcattaagtgatcaaggattccttgttgaaagggaactagagccaggcaaacctttggtaaaacaagcagtcatgttcctaagggactcagtccgatgtgctttagcaactatcaaggcaggagtcgagccggagatctcccgaggtggctgtacccaagatgagttgagtttcaccctcaagcacttgctatgtcgacgtctctgtataattgctctcatgcattcagaagcaaagaacttggtcaaggtcagaaatctcccagtagaggaaaaatctgctttactataccagatgttggtcaccgaagctaatgcccggaaatcaggatctgctagcatcatcataggcttaatttcggcacctcagtgggatatccataccccagcactgtactttgtatcaaagaagatgctaggaatgctcaaaaggtcaactacaccattggatgtaaatgatctgtctgagagccaggaccttatgccaacagagttgagtgatggtcctggtcacatggcagagggatttccctgtctatttagtagttttaacgctacatatgaagacacaattgtttataatccgatgactgaaaagcctgcagtacatttggacaatggatccaccccatccagggcgctaggtcgccactacatcttgcggcccctcgggctttactcgtctgcatggtaccggtctgcagcactcttagcatcaggtgctctcaatgggttaccggagggatcaagcctatacttgggagaagggtatgggaccaccatgactctgctcgaacccgtcgtcaagtcctcaactgtttattaccacacattgtttgacccgacccggaatccctcacagcggaattacaaaccagagccgcgagtcttcactgattccatctggtacaaggatgacttcacacgaccgcctggtggcattgtaaatctatggggtgaagatgtgcgtcagagtgacgtcacacagaaagacacagttaatttcatattatcccggatcccacccaaatcactcaaactgatccatgttgacattgaattctcaccagactccaatgtacggacactactatctggttactcccattgcgcattattggcctactggctattgcaacctggagggcgatttgcggttagggtcttcctgagtgaccatctcttagtaaacttggtcactgctattctgtctgctttcgactctaatctactgtgtattgcatctggattgacacacaaagatgatggggcaggttacatttgtgctaagaagcttgccaatgttgaggcatcaaggattgagcactacttaaggatggtccatggttgcgttgattcattaaagatcccccaccaactagggatcattaagtgggctgaaggtgaggtgtctcggctcacaaaaaaggcagatgaagaaataaattggcgattaggtgacccggttactagatcatttgatccagtttccgagttaataatcgcacggacaggggggtctgtattaatggaatatgggactttcattaatctcaggtgttcaaacctggcagatacatataaacttttggcttcaatcgtggagaccaccttgatggagataagggttgaacaagatcaattggaagacaactcaagaagacaaattcaggtggtccccgcctttaatacgagatccggggggaggatccgtacattgattgagtgtgcccagctgcaggttatagatgtcatatgtgtaaacatagatcacctcttccccaaacatcgacatgttcttgttacacaactcacttaccagtcagtgtgccttggagacttgatcgaggggccccaaattaagatgtatctaagggccaggaagtggatccaacgtagaggactcaatgagacaattaaccatatcatcactggacagatatcacgaaataaggcaagggatttcttcaagaggcgcctgaagttggttggcttctcgctttgcggcggttggagttacctctcactttagttacttaggttgttgatcattgtgaaaaatcggagtcggaatcgcaaataaaaacatacaaaattgcaaatttacaataatcgcattaatatttaataaaaaatatgtcttttatttcgt

SEQ ID NO: 9 - DNA, 16236 bp, Avian paramyxovirus 6
accaaacaaggaaaccatatgcttggggactttacgagagcgcttgtaaaaccgtgagggggaagctggtggactccgggtccggagtcggtggacctgagtctagtagcttccctgctgtgtcaagatgtcgtcagtgttcactgattacgctaagctgcaagatgcccttgtggccccttcgaagaggaaggtagatagtgcaccaagcggattgttaagggttgggatccctgtgtgtgtcctactctccgaagatcccgaagagcgatggagcttcgtttgcttttgcatgagatgggtggtgagcgattcagccacagaagcgatgcgtgttggtgcaatgctatccattctcagcgcacacgccagcaatatgcggagccacgttgcacttgcagcgaggtgtggtgacgccgacatcaacatacttgaggttgaggcaattgaccaccagaaccagaccattcgcttcactgggcgcagcaatgtgactgacgggagagcacgccagatgtacgcaattgcccaagatttgcctccttcctataacaatggcagcccttttgtaaatagagacattgaggacaattatccaactgacatgtctgagctgctcaatatggtttacagtgtcgcaactcaaatctgggtggcagctatgaagagcatgactgctccagacacatcctcggagtctgaggggaggcggctggccaaatacatccagcaaaacagagtaattcggagcacgattctagctcccgcaacccgcggtgaatgcacccgaataatacggagctccctagtcatccgccacttcctaataactgagatcaagcgtgccacatcaatgggttccaacacgacacgatattatgccacagttggggatgccgcagcttacttcaagaatgcgggtatggctgcattcttcttaactctgaggtttggaattgggaccaagtactccacacttgcagtttcggcgctgtctgctgacatgaagaaactccagagcttgatccgagtataccagagcaaaggtgaggatggaccctacatggcatttctggaagactccgaccttatgagcttcgcccctggaaactatccactcatgtattcatatgcaatgggagtagggtccattcttgaggcaagtattgctagatatcagtttgcgcgatcattcatgaatgacacattctatcgattgggtgttgaaactgcacaacgaaaccaaggttcacttgatgagaatttagcaaaggagctgcaactatccggggctgaacgaagggctgtgcaggaacttgtgaccagcctggatctagcaggagaggccccagtgccccagcgccaaccaacattcctcaatgaccaggagtatgaggatgatccccctgctaggagacagagaatcgaggatactccagacgatgatggagccagtcaagctccacccacaccaggagcaggtctcaccccatactctgataatgccagtggcctggacatctaaatgaccactactcaatatgacaagtaatcaaggttgatccaaagcatgcaaatccaacactacaatcgacaacaaaatcacatgtagactttaagaaaaaacaagggtgagggggaagttcctggtgcgcgggttgggcccctagtgactcagccagcaccatggacttctccaatgaccaagagattgcagaattactcgagctgagttcagatgtgataaagagcatccaacacgccgagacccagccagcgcacactgtcggcaaatctgccattcggaaaggaaacacatccgagctgcgagcagcctgggaagccgagacacaaccagcccgagcagaaaacaagcccgaggaacacccagagcaagccgcccgggatctcgacagcaagggcaacacggaaagcccacaactacgatccaatgcagatgagacaccccaaccagaaagccacgacaggcaagccactgccccatccccagacaccacaataggggtcaacgggactaatggacttgaagctgctctaaaaaagctagaaaaacaagggaaaggtcctgggaaaggccaagtggatcgcaacactcctcagagagatccaaccactgcttcgggttcaaaaaaggggaaagggggcgagccaaggaacaatgcccttcatcagggccacccacaggggaccaacctgatcctgcccactcagaagccctctcatgccagactggcgcagcaagcatcacaggagataactcgccatgcactgcaaccccaggattccggcggcatagaagggaattctccatttcttggagacacggccagtgcatcttggctgagtggtgcaacccagtctgcgcacccgtcacacctgaacccagaacattcaaatgcatttgcgggagatgccctcgggtatgcatcaactgtcgcaatgatagtggagactctgaaatttgtagttagcaggttagaagcacttgagaatagggtggcggagcttaccaagtttgtctctcccattcagcaaatcaaagcagacatgcagattgtaaagacatcctgcgctgtcattgagggccaacttgccacagtgcaaatattggagccgggccactcatcgatccgctcacttgaagaaatgaagcaatataccaagccaggggttgtcgtccaaacagggacgactcaagacatgggcgccgtcatgagggacggcacgatcgtgaaagatgctcttgcccgcccagtcaatccggacaggtggtcagcaacaatcaacgctcaatcaacaacaacaaaggtgactcaagaggatataaagacagtgtatacactattggacaattttggcatcaccggcccgaaaagagcgaaaatcgaggcagaactggctaatgtcagtgaccgggacgcactagtaaggataaagaaacgtgttatgaatgcataaacagcaagaagatcacaacaatcagtacagatgacatcccaatatcagatcatgattctattgccaaatcacagcatttttttctcctgatcacacctaacaatttgcttcagacacccttgacactgattaataaaaaagtgagggggaactggtggtgtccggactgggccatccagagtcacccagtccgaaccaaacacccgccagttcctccgccggcacagcgcgccaccaactgccccaactccaaccatggccacatcagaactcaacctctacatcgacaaagactcaccccaggtgagattgctagcattccccatcatcatgaaacccaaagaaagtggggttagagagctgcaaccgcaattgaggacccagtacctcggtgacgttaccggaggaaagaaaagcgcgatatttgtgaattgctatgggttcgtggaagatcacggggggcgagacagcggattctcacccatcagcgaggaatccaaaggatcgacagtcactgcagcttgcatcactctcggcagcatcgagtatgatagtgacatcaaggaggtggcaaaggcctgctataatcttcaggtgtcagtcaggatgtccgctgattcaactcagaaggtagtttacacaatcaatgccaaacctgcactgttgttctcctcccgtgttgtcagggctgggggttgtgtggttgcagcagaaggtgcaatcaagtgccccgagaaaatgacatctgatcgcctctacaaattccgcgtaatgtttgtgtcattgaccttcctacatcgcagcagcctttttaaagttagccgtacagtgctgtcaatgaggaattctgctctaatagcagtacaggccgaagtgaagctggggttcgatctgccactggaccatccgatggcaaaatatttgagcaaagaggatggacagctatttgcaactgtgtgggtacacttgtgcaactttaagcgcacagacagacgcggagtagaccgatcggtggagaacatcaggaacaaagtacgagccatggggctgaagctcaccttgtgtgatctatggggtcccacacttgtttgtgaagccacggggaagatgagcaagtacgcgctaggtttcttctcggagactaaggttggctgtcacccaatctggaaatgcaactcgactgtcgcaaagatcatgtggtcatgcacaacttggatcgcatcagcaaaggccatcatacaggcctcctctgctcgtaccttgttgacatcagaggacatagaagccaagggggccatctccactgacaagaagaaaacagatggattcaatcccttcatcaagacagcaaagtagtcatctggatttcatcaatgaacccactggcctatgttcagctgtaccttccttgataatcactaaatcaatacacagagtgccatttgattaagatattgattgtgccagtatgtggatcacttatactttgaagattgaccttcctagctgttcctcccttagaagtcctgtcatattaatcaaaaaaatcagtttgctggtaaaatagtatgctgcaggatccaatacctcccaccaatgagcagccgagggggaaggcatgggagcccgactggggccctttacaatggcacccggccggtatgtgattattttcaacctcatccttctccacaaggttgtgtcactagacaattcaagattactacagcaggggattatgagtgcaaccgaaagagaaatcaaagtgtacacaaactccataactggaagcattgctgtgagattgattcccaacctacctcaagaagtgcttaaatgttctgctgggcagatcaaatcatacaatgacacccttaatcgaattttcacacctatcaaggcgaatcttgagaggttactggctacaccgagtatgcttgaacacaaccagaaccctgccccagaacctcgcctgattggagcaattataggcacagcagcactggggctggcaacagcagctcaggttacagctgcactcgcccttaaccaggcccaggataatgctaaggccatcttaaacctcaaagagtccataacaaaaacaaatgaagctgtgcttgagcttaaggatgcaacagggcaaattgcgatagcgctagataagactcaaagattcataaatgacaatatcttaccggcaatcaataatctgacatgtgaagtagcaggtgctaaagtaggtgtggaactatcattatacttgaccgagttaagcactgtgtttgggtcgcagataaccaatccagcactctccactctatccattcaagccctcatgtcactctgcggtaatgattttaattacctcctgaacctaatgggggccaaacactccgatctgggtgcactttatgaggcaaacttaatcaatggcagaatcattcaatatgaccaagcaagccaaatcatggttatccaggtctccgtgcctagcatatcatcgatttcggggttgcgactgacagaattgtttactctgagcattgaaacacctgtcggtgagggcaaggcagtggtacctcagtttgttgtagaatctggccagcttcttgaagagatcgacacccaggcatgcacactcactgacaccaccgcttactgtactatagttagaacaaaaccattgccagaactagtcgcacaatgtctccgaggggatgagtctagatgccaatatacgactggaatcggtatgcttgaatctcgatttggggtatttgatggacttgttattgctaattgtaaggccaccatctgccgatgtctagcccctgagatgataataactcaaaacaagggactcccccttacagtcatatcacaagaaacttgcaagagaatcctgatagatggggttactctgcagatagaagctcaagttagcggatcgtattccaggaatataacggtcgggaacagccaaattgccccatctggaccccttgacatctcaagcgaactcggaaaggtcaaccagagtctatctaatgtcgaggatcttattgaccagagcaatcagctcttgaatagggtgaatccaaacatagtaaacaacaccgcaattatagtcacaatagtattgctagttatcctggtattatggtgtttggccctaacgattagtatcttgtatgtatcaaaacatgctgtgcgaatgataaagacagttccgaatccgtatgtaatgcaagcaaagtcgccgggaagtgccacacagttctaacagtatagctagtcctaatgattaaaccatatacttgattacataataacactatgtcaagggatgacattaatgagactccttattctctctcaaaccgagacagtgatccatcaagaatgcaacgatcctaccttctctgctttaatcaaaaaatgcagaataatctaacagcccaaccaaaccacccaggagagaacgcctgaggggggaaggaggttgactacaacctctactgatcagaggttgtagtatcaattcttaacaacccccaagatgagaccacaagtggcaatttggggcttgcgcttattggctaccggcctagctatggtctccttagtgttctgcctaaaccaggtaatcatgcaggtgctaattagggacattagaggcttgttgacatcctcggacatcaagactacacatgaggcgctgcgtgagcatctctcatctattactcttttcatgtcgtttgcgttgacttgctcaataagtgggtgtgttcttagcctggtcgccttatatccaagcaagaatactagcggcactaatcctcagccgcaagtagaggaggctagatcggaaaacctgtctcactcttccatgcacacgatcaataggccagcaacccctcccccaccgtattatgttgcaatacagctcagcgctgagatgcaacctgggtaccattcaagtgattgatccccttgacgcactggcagagtctaccccaccaagatccgttcttgtcctacttgtttgatttaagaaaaaattgtaatttatacagaaagataatagctgagggggaagcctggtgtcaccgctggtgaccattccccagccggtggcaatggcttcctcaggcgatatgagacagagtcaggcaactctatatgagggtgaccctaacagcaaaaggacatggaggactgtgtaccgggttgtcaccatattgctagatataaccgtcctttgtgttggcatagtggcaatagttaggatgtcaaccattacaacaaaagatattgataacagtatctcatcatctattacatccctgagtgccgattaccagccaatatggtcagatacccatcagaaagttaacagtattttcaaggaagttggaatcactatccctgtcacactcgacaagatgcaagtagaaatgggaacagcggttaacataatcactgatgctgtaagacaactacaaggagtcaatgggtcagcaggatttagcattaccaattccccagagtatagtggagggatagacacactgatataccctcttaattcacttaatggaaaggctctagctgtatcagacttactagaacacccgagcttcataccgacgcctaccacctctcacggttgtacccgcattcctacattccacctagggtaccgtcattggtgttatagtcacaacacgatagagtctggttgtcacgatgcaggagaaagcattatgtacgtatccatgggtgcggtaggggtcggccatcgcgggaaacctgtgtttacgacaagtgcagcgacaatcctagatgatggaaggaacaggaaaagttgtagcatcatagcaaaccctaatgggtgtgatgtcttatgcagcttggttaagcagacagaaaatgaaggctacgctgaccctacaccgaccccaatgatccacggtaggctccacttcaatggcacatacactgagtctgaacttgaccctggcctatttaataaccattgggtcgctcaatatccagcagttggtagcggtgtcgtcagccacagaaaactatttttcccgctctacggagggatatcaccgaagtcaaaactgttcaatgagctcaagtcatttgcttactttactcataatgctgaattgaaatgtgagaacctgacagagagacagaaggaagacctttataacgcatataggcctgggaaaatagcaggatctctctgggctcaaggggttgtaacatgtaatctgaccaatttagctgattgcaaagttgcaattgcgaacacgagcaccatgatgatggctgccgaggggaggttacagcttgtgcaagataagattgtcttctaccaaagatcctcatcatggtggccagtcctaatatattatgatatccctattagtgaccttatcagtgccgatcatttagggatagtgaactggactccgtatccacagtctaagtttccgaggcccacctggacaaagggcgtatgtgagaaaccggcgatatgccccgctgtatgtgtaacgggtgtttaccaagatgtttgggtagttagtatagggtcacagagcaatgagactgttgtggttggcgggtacttagatgctgcagcagcccgtcaggatccatggattgcagcagctaaccagtacaactggctggttaggcgtcgcctctttacatcccaaactaaagcagcatactcatcaaccacttgcttcagaaacacgaagcaggatagagtgttctgcctgactataatggaagtcacagacaacctactcggagactggaggatcgccccgctgttgtatgaagttactgtggctgataagcagcagggcaatcgcaattacgtgcctatggggagggtggggacagataagttccaatattataccccaggtgacagatatactcctcagcattgatgactcactgcagcttatacataacaattttctcatttcctctattcgcagagtgaatcagtagaatgacggtcagtgattgaccaagctcaattagataatgaagtgcagcccgcaattgtcttgatttaataaaaaattgaggggctgttataacatagcagactgacggggcaagacccgctgagaaaaaaaatgcagtgagggggaaggcaggctgagatcacgtcccagttgtagccttccccgattcaatttacttagtattaacaagtcaattctgctcacagaggtcatctctaagggccgctgtgatggatccacaagtccaaatacaccatatcatcaagccagagtgccatctcaactcacctgttgtggaaaagaaactgacattattatggaagctcacaggtttaccgttgccacccgaccttaacggttgcgtcacacacaaagacgtgacgtgggatgaagtgctccggttggaggctaatttgacgaaggagttacggcaattagtacgaagcctgaccaatagaatgcatgaaaagggggagttcattgacacatataaacctttatgtcatccacggacattaagttggttgaccaatatcaacttgatcaagagtgacaacattctagcaagccacaagaaaatgttgatccgaatcggcagtatgctgcatgaaccaacagaccaatcgtttgtcactcttggcaggaaattagcaggcgacccttgcttgttccatcaactaggccatctacctggatgcccacctaattccagatttgaagaacaggtaggagactgcagtttgtggtcacccataagcgatccagctctagtcacaggtggtgaatacgctaactgtgtgtatgcgtggtacttaatacgtcagaccatgcggtacatggccctccagagaaagcaaacaagagtgcaatcacagcagaatgttctaattggatcagatactatcgtgggaatccatccagaattagtgataattactggaattagagacagggtattcacctgtttgacttttgatatggtgctaatgtatgcagatgtggtggaaggtcgtgccatgacaaagttggttgcactcactgagccaacaatggtagaagtcattcagagagtcgaaaaattgtggttcttagttgacaacatcttcgaggaaatcggtggtgcaggttacaatattgttgcatctctggagagcttggcatatggtactgttcaactgtgggataaatcactggaacatgctggtgagttcttttcattcaatcttaccgagataaagagtgagctagagaaccatttagatcctggtatggcatttagagtagtcgagcaggtgcggttgctatatactggactaagtgtgaaccaagcaggtgagatgttatgcattttacgtcactgggggcatcccttactatgcgctgtgaaggcggcaaagaaagtcagagagtcaatgtgtgcaccaaaattaacctctctagacaccacactcaaggtgttagcattctttattgcagatatcatcaatggacatagacgatcacattcagggttatggccaagcgtcagacaggagtcattagtgtctccattgctccagaacctctatagagaatctgccgagcttcaatacgcagttgtgcttaagcactatagagaagtatcccttatagaattccaaaaaagtattgattttgacttagttgaagatctaagtgtgttccttaaggataaagccatttgtcgaccgaagagtaactggttagctgtattcaggaaatccctactccctggacatttgaaagataaactgcaatctgagggcccttctaaccggcttctgcttgactttttgcaatcaagcgaatttgacccggctaaagaattcgaatacgtgacatcgctggagtatcttcaggatccagagttctgcgcatcttattccttaaaagagcgggaagtcaaaactgatgggcgcatatttgcaaaaatgactagaaaaatgaggaactgccaagtcttgttagagagtctgctcgcatgccatgtatgcgattacttcaaggagaacggagtagtacaagagcaaatcagtttaacaaaatcactgcttgcaatgtcgcaacttgctcctcgtgtgtctgagtatcaagggagagttctccgctcgactgataggtgcagtagagctacagccacacctagtcaggacacaggcccaggcgagggggtcaggcgacggaaaacaattatagcatcattcttgactactgacctacagaagtattgtctcaattggaggtacaccgtaataaaaccttttgcccagaggcttaaccagttatttgggataccccacggctttgagtggattcacctccgcttgatgaacacaactatgtttgtaggagacccacataatgtccctcagttttcatcgacacacgacttagaatcccaagagaacgatggaatatttattgtgtcacctcggggtggtatagaagggctatgccaaaaaatgtggaccatgatctccattgcggcaattcatctagcagccacagaatcgggttgtcgggttgcatccatggtccagggggacaaccaagcaattgcaattactacggagatcgaagagggtgaggacgcgtctgtagcatcaataaggttgaaagagatatctgagaggttctttagggtgttcagagagatcaacaggggtataggacacaacttaaaagtccaagaaacaattcatagtgagtcattcttcgtgtactcaaaacggatcttctttgaggggaagatcctcagccagctactgaaaaatgcaagcaggttggtgttggtatccgagactgtgggtgagaattgtgttggcaattgctcaaatatcagttccacagttgctagactcattgaaaatggattagataagagagtcgcatgggggctcaatatcctgatgatcgtaaaacaaattctttttgacattgatttttccttggagcctgaaccatctcagggcttgagtcatgctattcgccaagacccaaacaacatgaaaaacatctctatcactcctgctcagttaggtggattaaattttctggccctatctcggctatttacaaggaacataggagaccccgtctcatcagccatggcagatatgaagttctatatacaggtcggattattatcccctcatctgctgaggaatgcaattttcagagaacccggagatggaacatggacaacactgtgtgccgacccgtactcattaaaccaaccatatgtgcaattaccaacgtcatacttaaaaaagcacacacaacgtatgctgctcactgcctcaacaaaccctttattgcaaggtacccgggtagagaatcaatacactgaggaagaaagactagcaaagttccttctggaccgagaattggttatgccacgtgtggcacatacagtctttgagaccactgttgccgggagacgaaagcatctgcaagggttaattgacactacaccgactattattaaatatgcccttcatcaccaccctatttctttcaagaaaagtatgctgatatcatcttactcagctgactacattatgtcgtttattgagactatcgcaacagtggaatacccaaagcgtgacaccatgcagctctggaacagaggactaattggtgtcgacacttgcgcggtcacacttgcggattacgcaagaacatattcgtggtgggagatcctgaagggtaggtcaataaagggagttaccacacctgatacattagaactttgctctgggagcttaatagagcaaggccatccatgttctcagtgcacaatgggtgatgaatccttttcatggttcttcctcccagggaatattgatattgaaagaccggacttttctagggtggcccagagaatcgcttatgtcggctcaaaaacggaagaaaggcgggcagcttcgttgacgacaatcaaagggatgtcaactcaccttagggcggcactaagaggggcgagtgtttacatctgggcgtatggagacagcgacaaaaattgggacgacgctacaaagcttgctaacacaagatgtgtaatatctgaagaccatctgcgtgccctttgcccaatcccgagttcagcaaacatacagcataggctgatggatgggataagcgtaacgaagttcactcccgcatccctagcaagagtgtcatcgtatattcatatttcgaatgaccggcatcagagtagaattgacggtcaagtgatcgaatcaaatgtgattttccaacaagttatgcttctcggtctcggtatttttgagacatttcaccccttgtctcacaggtttgtgactaaccccatgacactccacttacacacagggtactcgtgttgcataagggaagctgataatggtgatttcttagaatccccggctagtgtaccagacatgactatcacgactggtaataagttcctttttgaccccgtgcccattcaagatgacgatgctgcaaaactacaggtatcttcattcaagtactgtgagatgggcctcgaagtgcttgacccaccaggacttgtaaccctactatctctagtgactgcacgtatctctattgatacatctataggggagagtgcatacaactcgatacacaatgatgctattgtctcattcgacaattccatcaattggatatctgagtacacatactgtgatcttagactactggcagtagcaatggctcgggagttttgtgacaacctctcttatcagctttactatctgagggttaaagggcgacgggcaatccgggattatatccgccaagccctctcgaggataccagggttacaacttgctaatatagccttgactatatctcatccgggaatttgggcaagactgaggctaattggggcagtaagtgctggaaatagtcccatcagtgcaaccgtaaattatcctgctgctgtgtgtgagctcatattatggggttacgaacaatatactgcacaactactagatggttacgagttagaaattatagtcccgaattataaggatgatgacctgaacaggaaggttgaacatatactagcaagacgggcttgcctgctgagtctgctgtgtgagtatccaggaaaatacccgaatattaaagaccttgaacctattgagaaatgcactgctctgtctgacctgaataaattgtggatggcgacagatcacagaactcgggaatgtttttccgggatatctcagatatttgattcccccaaattaaatccgttcatcactaatctttacttcttgagtagaaagctgctcaacgcgattataagcagcacggactgtagggcctacgttgagaacctttatgaagatatcgacattgaactaacatctctcactgaggttttgcccttaggagaggatgatcaaatgatcactgggcctctgcgctttgaccttgaactaaaagaactcaccccggattttactatcacttggtgttgttttgactctacagcagcactgatgtcacggtgcattaatcatgccacagaaggcgcagagcgctacatccgaagaacggttgggacagcttcaacatcttggtataaagcagcaggaatattaactacacctggctttctcaacctccctaaaggcaatggcttatatctagctgagtcatcaggggccatcatgactgtgatggagcatcttgtctgctctaataaaatatggtataacaccttgtttagcaatgagctcaacccacctcagaggaattttggtcccaacccaattcaatttgaagaaagtatcgtgggtaaacatattgcagccgggattccttgcaaggcaggacatgtgcaagagtttgaggtactttggagagaggtagatgaagagacagatctgacctccatgagatgtgtgaattttatcatgtcgaaagttgaacagcactcgtgtcatattgtatgctgtgacttagaattggctatggggactcccttagaagtggcccaatctgcatatacgcatattgtaaccctcgccttgcattgcctaatgattagcggaaaattagtactaaagttgtatttctcacaaaatgccctcttacaccatgttctctctttattgcttgtattgccattccatgtaacaatccacactaacggttattgctctcaccgaggctctgaagggtatatcattgccacgagaacaggagttgctctgggttcaaatgtgtcccaagtactaggtggtgtgactgagatggtacggaaaggtcagacccttgtccctgtaaaggtacttacagcgatctccaatgggttcagaactgtgtcaagctctttaggcagactaaggggtgagctctattcgccatcgtgtagcattccgcagtcagctaccgacatgttcctcattcaacttggagggaaggtgcagtcagattggaatacgaactctcgaggctatagagtgggtgagactgatctcgtattacaggacattatatcaatattgagcacactacttaaagaaataatacacgtaagggaatccagggagtcagtggacagggttctgttgctcggggcatacaacctacaggtgtctggaaaagtaagaacaatggccgcggctgcaacaaggaacatattgcatctacatatagttagacttattggagactcaatgtccaatgtaaggagactagtacctctgctagataagggctttatagtaatatcagacatgtatagtgtgaaagatttcttgagaaaaactgagtcccctaagtacttcttaaacaagctaggcaagagcgagattgcacagctatttgagatagagtccaagattattctgagcagggcagagatcaagaatattttgaagacaatagggattgtggctaaacagcactcagagtgatctctccaaccttgcaccatttgaattctggactgtggacgcgcatgcctaagcgcaccaacttgccgtgacgattgatgtaatccttgatatgaactactaatcatttggaatttatttacttcccgaaatcacccatagaccggaatcgataccggagattattttttaataaaaaacctggaaagtcgacaaggatcatagtcaaaaagcttatgatttccttgtttggt

SEQ ID NO: 10 - DNA, 15480 bp, Avian paramyxovirus 7
accaaacaaggactgcataagcagtgtaaaacttttaataaaaaataactttcgtgagggtgaatcgatcatcgctcgaagccgatatcgactcacccaaattagctgcttgtataaggatccgaatatcaattggaatcatgtcatcgatttttactgattataccaatttgcaagagcaattagtcagaccggtaggccggaaggttgataatgcttcaagtggcttgttgaaagttgagataccagtctgcgtcctgaattcacaggacccagttgagagacaccagttcgcagtattatgtacaaggtggatctcaagttcaattgccacaactcctgtcaagcaaggtgccctgctttctcttctcagtttgcacacagaaaacatgcgagcgcatgttctattagcagcccggtcaggagatgctaatataacaattctagaagttgatcatgtagatgttgaaaagggagaattacaatttaatgcaaggagtggtgtctcatctgataaagctgatcggctgctggctgtcgcaatgaatcttattgcaggttgtcagaataactcaccatttgtcgacccatcgattgagggtgatgaaccaactgatatgactgaatttttagagctggcttatgggttagcggttcaagcatgggtagctgcaataaagagtatgacggcaccagatactgctgcggagagtgaggggcggcgattagcaaaataccagcagcaaggtcgtttaacacgacgtgctgctcttcaagcaaccgtgaggggggagttgcagcggataatcaggggttctctggtagttcgacacttccttataggagaaatcagaagagcaggaagtatgggagaacagacaacagcctattatgccatggtgggagatgtcagccaatacataaagaattcaggaatgactgcattcttcctgacattacgatttggggtgggtaccaagtatcctccccttgcaatggctgcattttcaggagatctcactaaactccagagcctgatcagactatatcgaaataaaggtgacatagggccttatatggccctactcgaagatcctgacatgggcaactttgctcctgcaaattacaccttgctctattcatatgcaatgggcattggttctgtattggaggctagtatcggtagataccagtatgcgagaacattcctgaatgaatcattctttaggttgggggcctcaactgctcaacagcaacaaggagcactggatgagaaattggctaacgagatggggctatcagaccaggcaagggcagcagtttccagattagttaatgagatggatatggatcagcaagtagcccccacaccagttaatccagtctttgcaggagatcaagcagccccacaggcaaatcctccagcccaaccaagacagaatgacacaccacagcagcctgctcctcttcagcagccaattcgaattgccatgcctcaaaattatgatgatatgccagacttagagatgtagacagaaccccaatcaagcaacaattggcattaagatctaagctgaatgtatgagcacacgagtacccaagtatatttgttagcagttgcatgaaatcattatccatattattgatttgcaatatagaaaattactgataaacaattaagaatcatttaataaaaaaattccacaaaaattaaaaaaattgtgagggggaacacctttcagtcggtcaactgctgctaataacctgcaattatcacgtggattgaatatggaattcagtaatgatgccgaggttgccgcgctcctggatcttggagatagcatcattcagggcattcagcatgcaacaatggctgatccgggaacactagggaagtcagctattcctgcaggtaataccaaacgcttagagaaattatgggagaaagaatctgttcctaatcatgataatatgattcactcttccatgagtgcagaacctataagcggggaactacctgaggaaaacgctaaaactgaaccaacagggactcaagaaatgccagaacaaattcaaaagaatgacaatctccaacctgcatccatcgataacatattgagcagcattaatgcattagagtcaaaacaggttaaaaaagggttagtgctatcgccccaatcactgaaaggtgtgtcccccttaatcaagaaccaggatctgaagaacaccatgcaggacctggaaaccaaacccaaggctgtaacgactgtaaatccattagcaaaccgacaagtgtcacctggaagcctggtcatagacgagagtattcctttgcttggagtgcaggaacaaacaaatttattgtctcctcgtggtgtaacccaacttgcgccccaatcagaccctatcctacagtcgaacgatgcaggtgcgggaattgcccaaaattctgccctggatgtcaatcagctctgggatgtaatcaatcagcaacacaagatgctgataaacctacaaaatcaagtaacaaagatcactgagctggttgctttaattccaattcttcgaagtgatattcaggctgtaaagggaagttgcgcattattagaagcacagctagcatctataagaatactagatcctgggaacatcggggtatcttcattagatgatcttaaaacagcagggaaacaaagtgtagttattaatcaagggagctatactgatgcaaaggatctgatggttgggggaggattgattcttgatgaacttgctagacctactaaattagtcaatccaaagccacaacaatcttccaaaatattggatcaggcagaaattgaaagtgtcaaggccctaatccatacctacactcacgatgataagaagcggaacaaattcttaactgcacttgacaaggtgacaacccaggatcagctaactcgcatcaagcagcaagtattaaatcaatagatagacaattagcattcattcaagctatactcatttaagtgctttgattgtgttgcggaaactatattgagataatttagtcttacatgcaaaataacattaaaaattaattatgagcaatcttgatttttctaactcataatcaacctccttctctataaaggcatacttagtattgcaaaaagagaaaattaagaaaaaaagaaaaagaaaattgagggagaccgcttgatagatctgtgatcggtctcataacctcaaattaaaatggaatctatatctctggggttatatgttgatgaaagtgatccagcatgctcattacttgcattccccataatcatgcagactacaagtgaaggaaagaaggtcttacaaccgcaagtcagaataaaccgtctagggagtatatcgatagaaggagttcgggcaatgttcataaatacatatggcttcattgaggagaggcctacggaaaggacaggtttctttcagccaggcgaaaaaaatcagcagcaagttgtgacagctggtatgctgacattgggccaaataaggaccaatatagacccggacgaaattggagaggcatgcttgagactcaaagtgaatgctaaaaaatcagcagcaagtgaggagaagatagtatttagcattcttgaaaagcctcccgccctgatgactgcacctgtagtacaagatgggggcttaattgctaaagcagaaggatcaatcaaatgcccaggtaagatgatgagtgaaattcactactcatttagagtaatgtttgtgagtatcacaatgctggataatcagagcctatacagagtaccaacagccatcagctcgttcaaaaataaagctctatattctattcagttagaggtattgctggaagttgatgtgaagcctgagagcccccagtgtaaatttctagcagaccagaaagggaagaaagttgcttctgtatggttccatctctgcaattctaaaaagacgaatgccagcgggaaaccgagatcattagaggatatgagaaagaaggtccgagatatgggaatcaaagtgtctctggccgacctttggggccctacgatcatcgtcagggccacagggaagatgagtaaatatatgctaggatttttctctacctcagggacttcatgtcatccagtaacaaagagttcaccagatttggcaaaaatattatggtcatgctcaagcacaatcatcaaagcaaatgccattgttcaagggtcagtcaaagtcgatgtcctgaccctcgaagatatccaagtttccagtgctgcaaaaatcaacaaatcaggaatagggaagtttaatccatttaagaaataaagtcatatgcagattaaaatttgatcaagattggtcttagcaaattaactgaatgtaattataaaatacctcagtaaaatgctaatgaatcagtggatgatattgaattagcagattgaaaattaaagaaaaccttatgagggcgaatgagcttagatgatttaataaaggagactaatccaacatttccctcaaattaacaaaatcagaaagtaaaaagaaagggagcaatgagagtacgacctttaataataatcctggtgcttttagtgttgctgtggttaaatattctacccgtaattggcttagacaattcaaagattgcacaagcaggtattatcagtgcacaagaatatgcagttaatgtgtattcacagagtaatgaggcttacattgcactgcgcactgtgccatatatacctccacacaatctctcttgtttccaggatttaatcaacacatacaatacaacgattcaaaacatattctcaccaattcaggatcaaatcacatctataacatcggcgtcaacgctcccctcatcaagatttgcaggattagtagtcggtgcaatcgctctcggagtagcgacatctgcacaaataactgcagccgtggcactcacaaaggcacagcagaacgctcaagaaataatacgattacgtgattctatccaaaatactatcaatgctgtgaatgacataacagtagggttaagttcaataggagtagcactaagcaaggtccaaaactacttgaatgatgtgataaaccctgctctgcagaacctgagctgccaggtttctgcattaaacttagggatccaattaaatctttatttaaccgaaattacaactatctttggaccgcaaattacaaatccatcattgaccccattgtcaattcaggcattatacaccctagcaggagataacctgatgcaatttcttaccaggtatggctatggagagacaagtgttagcagtattctcgagtcaggactaatatcagcacaaattgtatcttttgataaacagacaggcattgcaatattgtatgtcacattaccatcaattgcgactctttccggttctagagttaccaaattgatgtcagttagtgtccaaactggagttggagagggttctgctattgtaccatcatacgttattcagcagggaacagtaatagaagaatttattcctgacagttgcatcttcacaagatcagatgtttattgtactcaattgtacagtaaattattgcctgatagcatattgcaatgcctccagggatcaatggcagattgccaatttactcgctcattgggttcatttgcaaacagattcatgaccgttgcaggtggggtgatagcaaattgtcagacagtcctgtgccgatgctataatccagttatgattattccccagaacaatggaattgctgtcactctgatagatggtagtttatgtaaagaacttgaattggaggggataagactaacaatggcagacccagtatttgcttcatactctcgtgatctgattataaatgggaatcaatttgctccgtctgatgctttagacattagtagcgaattaggtcaactgaataactcaattagctcagcaactgataatttacagaaggcacaggaatcattgaataagagtatcattccagctgcgacttccagctggttaattatattactatttgtattagtatcaatctcattagtgataggatgtatctccatttattttatatataaacattcaaccacaaatagatcacgaaatctctcaagtgacatcatcagtaatccttatatacagaaagctaattgatgaattaatttctaaaaaataatttgatgttctaataggagaatgcaatatcaatatgtccattataatatacttgattgattgaaagatctgataataatagtttataagacactaagtaagagttaaatgctaaagcaagttgattcctaaatttctgcacaataggaccatactatatcatattagataattaataaaaaacgccctatcctgagggcgaaaggccgatcattagtgactttaaccgttgctctcccaatttaaaatatatttcacatggagtcaatcgggaaaggaacctggagaactgtgtatagagtccttacgattctattagatgtagtgatcattattctctctgtgattgctctgatttcattgggtctgaagccaggtgagaggatcatcaatgaagtcaatggatctatccataatcaacttgttcccttatcggggattacttccgatattcaggcaaaagtcagcagcatatatcggagcaacttgctaagtatcccactacaacttgatcaaatcaaccaggcaatatcatcatctgctaggcaaattgctgatacaatcaactcgtttctcgctctgaatggcagtggaacttttatttatacaaattcacctgagtttgcaaatggtttcaatagagcaatgttcccaaccctaaatcaaagcttaaatatgctaacacctggtaatctaattgaatttactaattttattccaactccaacaacaaaatcaggatgtatcagaataccatcattttcaatgtcatcaagtcactggtgttatacccataatatcattgctagtggatgtcaggatcattcaaccagtagtgaatacatatcgatgggggttgttgaagtgactgatcaggcttacccgaactttcggacaactctttctattacattagctgataatctaaacagaaagtcatgtagcattgcagcaactgggttcgggtgtgatatattatgtagtgttgtcactgagacagaaaatgatgattatcaatcaccagaaccgactcagatgatctatggaagattattttttaatggcacatattcagagatgtcattgaatgtgaaccaaatgttcgcagattgggttgcaaattatccagcagttggatcaggagtagagttagcagattttgtcattttcccactctatggaggtgttaaaatcacttcaaccctaggagcatctttaagccagtattactatattcccaaggtgcccacagtcaattgctctgagacagatgcacaacaaatagagaaggcaaaagcatcctattcaccacctaaagtggctccaaatatctgggctcaggcagtcgttaggtgcaataaatctgttaatcttgcaaattcatgtgaaattctgacatttaacactagcactatgatgatgggtgctgagggaagactcttgatgataggaaagaatgtatacttttatcaacgatctagttcgtattggccagtgggaattatatataaattagatctacaagaattgacaacattttcatcaaatcaattgctgtcaacaataccaattccatttgagaaattccctagacctgcatctactgctggtgtatgttcaaaaccaaatgtgtgtcctgcagtatgccagactggtgtttatcaagatctctgggtactatatgatcttggcaaattagaaaataccacagcagtaggattgtatctaaactcagcagtaggccgaatgaacccttttattgggattgcaaatacgctatcttggtataatacaactagattattcgcacagggtactccagcatcatattcaacaacgacctgcttcaaaaatactaagattgacacggcatactgcttatcaatattagaattaagtgattctttgttaggatcatggagaattacaccattattgtacaatatcactttaagtattatgagctagatcctgttttaacattgaatcgtatgaacttataagactgaaggatgtctgttggtattaagcatcataaaacacggttgtttttgatttgacacctaatcgtactcaatactctccatagatttaatctaacagatttagatactattgatcatataggcatagatggtatatgggcaattagattgaactgagttaaatccgattgatacttatcaaattaagatctagattatttaataaaaaatctaagttagaaaatgagggggacctcattatggagttcagacaatctgatcaaataatacatcctgaagtgcatctagattcacctattattgggaataaaatactctatttatggcgaattacaggcttacctactccgcctgttcttgagcttaactctactatatcgcctgaagtctggacaaacttgaaagccaatgatcctagagtagcctttaaatgggacaaactaagaccacggttgctaacatgggcagcacatcaagggatatcactatcggatctgatccctattacacatcctgagtcattgcagtggttaacaacaatatcctgtcctaaaattgatgaaaattttgcgttaattaagaagtgccttcttagaacaagggactatacagcatcaggatttaagaatttattccaaatgatctcacagaaattgacgtcgacgaatattctattttgcgcagaaaatccgacaactccccccatctccgacgaagcatcctgggcattaaagaatcctgagcactggtttaatacaccttggtcatcttgttgtatgttttggttacatgtgaaacagactatgaggaacttaattagaatacaacgatctcaaccagaatcacaaagcatatacagtatcacggttgataacttgtttgttggattgactcctgacttgtgtgtcatagctgattctcaaagacaatcaattacagtactgtcatttgagtgtgtattgatgtattgtgacttaattgaaggtcgtaacaatgtttatgacctctgtcaattgtctcctgtgctaagtcctcttcaagatagaattttacttttactgagattaattgattctttagcatatgacatcggagcgccaatttttgatgtaattgcttctcttgaatctttagcatatggagctattcagctatatgattacgacacagaggcagccggtgattttttctcatttaatttaagagaaatttcccaggtcatagaagagagcaaatgtaggaatcaaacccatactataatcagtgcaattagtaagatttacacagggatcaatcctgatcaagcagctgaaatgctgtgtatcatgagactgtggggtcacccattgctttatgcatccaaggctgcatctaaggttcgcgagtcaatgtgtgcacctaaagttatccaatttgatgcaatgctgcttgtattagcattctttaagagaagcatcataaatggatatagacgaaagcatggtgggctatggccgaacatcatagttgagtcacttctttctgcagaacttgtcgcggcacatcatgatgcagttgaattgacagacacttttgttattaaacactatagagaagtagccatgattgacttcaaaaaatcattcgactacgatataggggatgacttaagtttatacctcaaggataaagcaatttgtcgacagaaatcagagtggcttaatatcttcaagggtcaattgcttgagcccgctgtacgatcgaagcgaattcgtggaataggtgaaaaccgattactgttacatttcttgaattcagtcgattttgatcctgaacaagaattcaaatacgtcactgatatggagtacctctacgatgaaacattctgtgcatcctattcactgaaggaaaaagaagtgaaaagagatggaagaatattcgcaaaaatgacaccaaaaatgagaagctgtcaagttttattagaggcattgttagcaaaacatgtaagcgaacttttcaaggagaatggagtctcaatggagcagatatccctcacaaagtcattggtagccatgtcacaattagctccccgagtgaatatgagaggtgggagagcagctagatcaacagacgttaaaatcaatcaacgaagggtcaagtcaatcaaagagcatgttaaatcgagaaatgattcgaatcaagagaaaattgtaattgcaggttatctgactactgatttacaaaaatactgcctcaattggagatatgaatcaataaaattatttgcaagagcacttaaccaattatttggaataccccatggatttgaatggatacacttaaggctcataagaagtacaatgtttgttggggatccttacaatcctcctgcatcaatccaatctttggatctcgatgaacagcctaatgatgatatttttattgtctcgccacgtggtgggattgaaggattatgtcagaagatgtggacactcatctcaattgcattaattcaagctgcagctgcaaaaataggatgtcgggttacaagtatggtacagggagataatcaggttattgctatcaccagagaagtgcgagtgggggaacctgtgagggaggcgtcacgagaactcagattattgtgtgatgagttcttcactgaattcaaacaattaaactacggaatagggcacaatcttaaagcaaaagaaactatcaagagtcaatcgttttttgtatatagcaagagagttttctttgagggaagagtgttaagtcagatattgaagaatgcctcaaaattgaatctaatttctgactgtctggctgaaaatacagttgcttcatgtagcaatatttcttctactgtagcaaggctaatagagaatggccttgggaaagacgtagccttcattttaaactttcagactattataaggcaactgatttttgatgaagtatatacgatttcattgaactatagtacagcaagacggcaggtgggaagcgagaatcctcacgcattggctatagccgctttgattcctggtcaacttgggggattcaatttcctaaacgttgctaggttatttacacggaatatcggggatccaatcacttgctcattgagtgatatcaaatggtttgcaaaagttggattgatgcctgagtacatccttaaaaacattgttttgagggcaccaggttcaggaacatggacaactttagtcgctgatccctactccttaaacattacgtacacaaaattgcctacgtcgtacctaaagaaacatacacagaggacattagttgctgattcccctaatccgttgcttcagggggtgtttctattaaatcagcagcaggaggatgaagcattatgtaaatttcttcttgaccgagaacaagtgatgccacgagctgcccatgtaatctatgatcagtcagttctcggccggaggaaatatttacaagggcttgttgatactacacagacaatcataaggtatgcactccaaaaaatgccggtatcatacaaaaagagtgaaaaaatccaaaattacaatctcctctacatacaatcactttttgatgaggtcttgacacagaatgtcattcatagtggattggatactatatggaaaagagatctaattagcattgagacctgttctgtcacacttgccaattttacgaggacttgctcgtggtctaatattctacagggcaggcaaattgttggagttacaactccagacacgatagaattgtgtaccggttctttgatttcttgcaacagtgcatgtgagttttgtagaattggagataaaagctactcttggtttcatacaccagggggtatctcatttgatacaatgagccctggcaatctgattcaaagagtgccgtacctaggatcaaagactgatgaacagcgagctgcctctctaacaaccatcaaggggatggattaccatctgagacaagctcttcgaggagcatcattgtatgtgtgggcatatggagagactgatcagaattggttagatgcgctgaagttagcaaacacccggtgcaatgtaacattacaagctttgactgcactctgcccaataccgagtaccgcaaatctacaacaccggcttgcggatggaataagtacagttaaattcacacctgcaagtttgtcacgaatagcagcttatattcacatttgtaatgaccaacaaaagcatgataacctagggaatagttttgaatcaaatctgatttaccagcaaataatgcttcttggaacaggaatatttgaaacaattttcccactatcagttcaatatatccacgaggaacaaacacttcacttgcacactggattttcctgttgtgtcagggaagctgacacaatgattatagatgagagcagaactggattcccaggattgacagtgactaagagtaataagtttttattcaaccctgaccctattcctgcagtgtgggcagataaaatattcacgactgaatttagattcttcgagtacaatatagagaatcaaggaacttatgaactaataaaatttctttcttcttgctgcgcgaaagttgttacagaatcgctagttcaggatactttccatagttctgtcaaaaatgatgcaataattgcgtatgacaattcaattaattacatcagtgagctacaacaatgtgacattgttctgtttagcagtgaacttggaaaggaattacttctagatttagcttaccagctgtactaccttcgaattagatcgaaacgaggtataattagttacttgaaggtactgctgactcggcttccaattattcagtttgcaccgcttgcgttgacaatatcacatcctgtaatctacgagcgattacgccaacggaggttggttatggaaccgttgcaaccttatttggcttcgatagattatgtcaaagccgcaagagagcttgttttgattggtgcttcttcttacctctcaatgcttgagacaggtttagataccacttacaacatatacagtcatttagacggggattcagagggcaagattgatcaggcgatggcaaggagactgtgcctaatcacattattagtgaatcctggatatgcattacctgtgatcaaaggactaactgcaattgagaaatgtagactattaacagattttttacaatcagatatcatttctgtttctttatctgagcagattgcaacacttattctaacaccaaagattgaagtgcacccgacaaatttatactatatgatgcggaagaccttgaatctaatccggtcacgagatgatacagttgtgatcatggcagaattgtataatatagatcaagagtctgcgataatgagggttgaatcagaagaggacggccctgtagacaaaatgaatcttgcacccatactaaggcttgtgccaatcacattcaaatcaatggacttgcatgccttaactgggctaggtagaaaagaggtggaactgatgggtagcccagtttgcaaaatcactcagagattagataagtacatctatcgcacaattggcaccatatctactgcatggtataaagcaagtagtttaatcgccagtgacatacttaaggggggcccattgggggacagcttatatttatgtgagggaagtggtagtagtatgacatgtttggaatattgtttcccttcgaaaacaatctggtataattcattcttctcaaatgagctaaatccacctcaacggaacatcggcccattaccaacacaattttgttcaagcattgtctatcacaatttgaatgctgaagtcccgtgctctgcagggtttatccaagatttcaaagtactctgggccgacaaatcagtggagactgatatttctacaactgaatgtgtgaatttcatcctaagcaaagttgaacttgaaacatgcaaattgatacatgcagaccttgatctacctattgagaccccaagatctgtctggatggcttgtgtcacaaatacattcattttgggaaatgccttattgaagtcaggagggaaattggtcatgaaattatatgcagtagatgagctcctcttttcatcttgcttaggattcgcatggtgccttatggacgatataaatatcctccgaaatggctacttcaatgacaaatcaaaggaatgctacctcattgggacaaaaaaggtgacaatcccgcaccagaaaatccaggatatccagcagcaaataaataagattgctagtcaagggttaagtgtcatacctgaagctgtaattcatgacatttacaaccagcttgaggacagtattagatgtgagaaaaaattcaaaaatgataatgcaccgacttggtccaatgggatcctcaattcgacagatctattactaataagacttggagggaaaccaattggggaatcactattagagttaacatccatacaaggcatggattatgatgatttaacaggggatataattcaagtaatagacacagcgctaaatgagattattcacctcaagtctgatacttcgagcttagatcttgtactgctaatgtctccttacaatctggcacttggagggaaaataagcacaattctgaaatctgttgttcaccagactctaatactcaggattatccaatctaggcagaataaggatataccattaaaaggatggttgtctctgttgaatcaaggagtcatctcactatcttcattgatcccgttgcatgattatctgaggaagagtaagttgagaaaatttatagttcaaaaattaggccaacaggaattacaagcattttggcagagcaggtctcaacaaatgctgagtagaagtgagaccaagttgctaataaaagtgctgagtgctgcttggaagggattgttgtaaaattgtaaatatacactgcatgtatataaattggttgctacccttatcagctaaccacaggtgtaaattttcatatggaatgcatatcaataaagataggcatttaaattatacaatgataacatattttaggttgacaacaatcattgatataatcaccaatagtagctctattacttatttgttaataataaatggtacactttgaatttaagaaaaaattagaattgctatattttatcgctatagtgggcctgtcggctgcgttagcggtaagacaaagaggacttgtcttttaaaaatttattaaaaaatcattaattgatcatattgctttccttgtttggt

SEQ ID NO: 11 - DNA, 15342 bp, Avian paramyxovirus 8
accaaacaaggaatgcaagaccaacgggaactttaaataaaacaatcgaatcattgggggcgaagcaagtggatctcgggctcgaggccgaaacactggatttcgctggaggttttgaataggtcgctataagactcaatatgtcatctgtattcaatgaatatcaggcacttcaagaacaacttgtaaagccggctgtcaggagacctgatgttgcctcaacaggtttactcagggcggaaatacctgtctgtgttacattgtctcaagaccccggtgagagatggagccttgcttgccttaatatccgatggcttgtgagtgattcatcaaccacaccaatgaagcagggagcaatattgtcactgctgagtctacattcagacaatatgcgagctcacgcaacattagcagcaaggtctgcagatgcttcactcaccatacttgaggtagatgaagtagatattggcaactccctaatcaaattcaacgctagaagtggtgtatctgataaacgatcaaatcaattgcttgcaattgcggatgacatccccaaaagttgcagtaatgggcatccatttcttgacacagacattgagaccagagacccgctcgatctatcagagaccatagaccgcctgcagggtattgcagctcagatatgggtgtcagccataaagagcatgacagcgcctgacaccgcatcagagtcagaaagtaagaggctggccaaataccaacaacaaggccgactggttaagcaagtacttttgcattctgtagtcaggacagaatttatgagagttattcggggcagcttggtactgcgccagtttatggttagcgagtgcaagagggcttcagccatgggcggagacacatctaggtactatgctatggtgggtgacatcagtctgtacatcaagaatgcaggattgactgcatttttcctcaccctgaagttcggggttggtacccagtatccaaccttagcaatgagtgttttctccagtgaccttaaaagacttgctgcactcatcaggctgtacaaaaccaagggagacaatgcaccatacatggcattcctggaggactccgatatgggaaattttgctccagcaaattatagcacaatgtactcttatgccatgggcattgggacgattctggaagcatctgtatctcgataccagtatgctagagactttaccagtgagaattatttccgtcttggagttgagacagcccaaagccagcagggagcgtttgacgagagaacagcccgagagatgggcttgactgaggaatccaaacagcaggttagatcactgctaatgtcagtagacatgggtcccagttcagttcgcgagccatcccgccctgcattcatcagtcaagaagaaaataggcagcctgcccagaattcttcagatactcagggtcagaccaagccagtcccgaatcaacccgcaccaagggccgacccagatgacattgatccatacgagaacgggctagaatggtaattcaatcacctcgacacatccacctatacaccaattctgtgacatattaacctaatcaaacatttcataaactatagtagtcattgatttaagaaaaaattgggggcgacctcaactgtgaaacacgccagatctgtccacaacaccactcaacaacccacacaagatggacttcgccaatgatgaagaaattgcagaacttctgaacctcagcaccactgtaatcaaggagattcagaaatctgaactcaagcctccccaaaccactgggcgaccacctgtcagtcaagggaacacaagaaatctaactgatctatgggaaaaggagactgcaagtcagaacaagacatcggctcaatctccacaaaccacacaagttcagtctgatggaaatgaggaggaagaaatcaaatcagagtcaattgatggccacatcagtggaactgttaatcaattagagcaagtcccagaacaaaaccagagcagatcttcaccaggtgatgatctcgacagagctctcaacaagcttgaagggagaatcaactcaatcagctcaatggataaagaaattaaaaagggccctcgcatccagaatctccctgggtcccaagcagcaactcaacaggcgacccacccattggcaggggacaccccgaacatgcaggcacggacaaaacccctgaccaagccacatcaagaggcaatcaatcctggcaaccaggacacaggagagaatattcatttaccaccttccatggcaccaccagagtcattagttggtgcaatccgcaatgtaccccaattcgtgccagaccaatctatgacgaatgtagatgcggggagtgtccaactacatgcatcatgtgcagagatgataagtagaatgcttgtagaagttatatctaagcttgataaactcgagtcgagactgaatgatatagcaaaagttgtaaacaccaccccccttatcaggaatgatattaaccaacttaaggccacaactgcactgatgtccaaccaaattgcttccatacaaattcttgacccagggaatgcaggggtgaggtccctctctgaaatgagatctgtgacgaagaaagctgctgttgtaattgcaggatttggagacgacccaactcaaattattgaagaaggtatcatggccaaagatgctcttggaaaacctgtgcctccaacatctgttatcgcagccaaagctcagacttcttccggtgtgagtaagggtgaaatagaaggattgattgcattggtggaaacattagttgacaatgacaagaaggcagcgaaactgattaaaatgattgatcaagttaaatcccacgccgattacgcccgagtcaagcaggcaatatataatgcataatattgtaattatacaaacaatcaatactgctgtcggttgcacccaccttagcaaatcaataatcttttaaaattgattgattaagaaaaaattgactacaataaggaaagaacaccaagttgggggcgaagtcacgattgaccacagtcgctatctgtaaggctcctcaccaaaaatggcatatacaacactaaaactgtgggtggatgagggtgacatgtcgtcttcgcttctatcattcccgttggtactaaaagagacagacagaggcacaaagaagcttcaaccacaggtaagggtagattcaattggcgatgtgcagaatgccaaagagtcctcgatattcgtgactctatatggtttcatccaagcaattaaggagaattcagatcgatcgaaattcttccatccaaaagatgacttcaaacctgagacagtcactgcaggactggtagtagtgggtgcaatccgaatgatggctgatgtcaataccatctctaatgatgcactagcgctggagatcactgttaagaaatctgcaacttctcaagagaaaatgacggtgatgttccacaatagccccccttcattgagaactgcaataactatccgagcaggaggtttcatctcgaatgcagacgaaaatataaaatgtgccagcaagttgactgcaggagtgcagtacatattccgtccaatgtttgtttcaatcactaaattacacaatggcaaactatatagggtgcccaaaagtatccacagcatctcgtctaccctactgtatagtgtgatgttggaggtaggattcaaagtggacatcgggaaggatcatccccaggcaaaaatgctgaagagggtcacaattggcgatgcagacacatactggggatttgcatggttccacctgtgcaatttcaaaaagacatcctctaagggaaagccgagaacgctagacgaactgaggacaaaagtcaaaaatatggggttgaaattggagttacatgacctatggggtccgactattgtggtccaaatcactggcaagagcagcaaatatgctcaaggatttttttcttccaatggtacttgttgcctcccaatcagcagatctgcaccagagcttgggaagcttctgtggtcctgctcagcaactattggtgacgcaacagttgttatccaatcaagcgagaagggggaactcctaaggtctgatgatctcgagatacgaggtgctgtggcctccaagaaaggtagactgagctcatttcaccccttcaaaaaatgatgcaggacatagtacagagaatgaaagggccatcagacgtgcgaaaaaaactaaatctgaaaaaaactgcccagactccacattaatctaggttgcagggaaataatacccgacatgcacaatactatcacggtcaccagcaatcagcaaagttgatcaatcactatataaggaatcaagtgggataacaattattaatccaatttcataattataaaaaattgctttaaaggttactgacgagtcgggggcgaaaccttgccacttaagctgcagtcaattttagaatctacatattgaattatgggtaaaatatcaatatatctaattaatagcgtgctattattgctggtatatcctgtgaattcgattgacaatacactcgttgccccaatcggagtcgccagcgcaaatgaatggcagcttgctgcatatacaacatcactttcagggacaattgccgtgcgattcctacctgtgctcccggataatatgactacctgtcttagagaaacaataactacatataataatactgtcaacaacatcttaggcccactcaaatccaatctggatgcactgctctcatctgagacttatccccagacaagattaattggggcagttataggttcaattgctcttggtgttgcaacatcggctcaaatcactgctgcagtcgctctcaagcaagcacaagataatgcaagaaacatactggcactcaaagaggcactgtccaaaactaatgaggcggtcaaggagcttagcagtggattgcaacaaacagctattgcacttggtaagatacagagctttgtgaatgaggaaattctgccatctatcaaccaactgagctgcgaggtgacagccaataaacttggggtgtatttatctctgtatctcacagaactgaccactatattcggtgcacagttgactaaccctgcattgacttcattatcatatcaagcgctgtacaacctgtgtggtggcaacatggcaatgcttactcagaagattggaattaaacagcaagacgttaattcgctatatgaagccggactaatcacaggacaagtcattggttatgactctcagtaccagctgctggtcatccaggtcaattatccaagcatttctgaggtaactggtgtgcgtgcgacagaattagtcactgttagtgtaacaacagacaagggtgaagggaaagcaattgtaccccaatttgtagctgaaagtcgggtgactattgaggagcttgatgtagcatcttgtaaattcagcagcacaaccctatactgcaggcaggtcaacacaagggcacttcccccgctagtggctagctgtctccgaggtaactatgatgattgtcaatataccacagagattggagcattatcatcccggtatataacactagatggaggggtcttagtcaattgtaagtcaattgtttgtaggtgccttaatccaagtaagatcatctctcaaaatacaaatgctgcagtaacatatgttgatgctacaatatgcaaaacaattcaattggatgacatacaactccagttggaagggtcactatcatcagtttatgcaaggaacatctcaattgagatcagtcaggtgactacctccggttctttggatatcagcagtgagatagggaacatcaataatacggtgaatcgtgtggaggatttaatccaccaatcggaggaatggctggcaaaagttaacccacacattgttaataatactacactaattgtactctgtgtgttaagtgcgcttgctgtgatctggctggcagtattaacggctattataatatacttgagaacaaagttgaagactatatcggcattggctgtaaccaatacaatacagtctaatccctatgttaaccaaacgaaacgtgaatctaagttttgatcattcaggccaaaacagagggtctaggctcgggttaataaaagttcaatcaatgtttgatttattaggctttccctactaattattaatgtatttgtgattatatgataacgttaaaagtcttaaatatttaataaaaaatgtaacctgggggcgacctatttacaggctagtatatattaggaagtcctcatattgcactataatctcaaacaattatattacctcgtatccaccttgtctaaagacatcatgagtaacattgcatccagtttagaaaatattgtggagcaggatagtcgaaaaacaacttggagggccatctttagatggtccgttcttcttattacaacaggatgcttagccttatccattgttagcatagttcaaattgggaatttgaaaattccttctgtaggggatctggcggacgaggtggtaacacctttgaaaaccactctgtctgatacactcaggaatccaattaaccagataaatgacatattcaggattgttgcccttgatattccattgcaagtaactagtatccaaaaagacctcgcaagtcaatttagcatgttgatagatagtttaaatgctatcaaattgggcaacgggaccaaccttatcatacctacatcagataaggagtatgcaggaggaattggaaaccctgtctttactgtcgatgctggaggttctataggattcaagcaatttagcttaatagaacatccgagctttattgctggacctacaacgacccgaggctgtacaagaatacccacttttcacatgtcagaaagtcattggtgctactcacacaacatcatcgctgctggctgtcaagatgccagtgcatctagtatgtatatctcaatgggggttctccatgtgtcttcatctggcactcctatctttcttactactgcaagtgaactgatagacgatggagttaatcgtaagtcatgcagtattgtagcaacccaattcggctgtgacattttgtgcagtattgtcatagagaaggagggagatgattattggtctgatactccgactccaatgcgccacggccgtttttcattcaatgggagttttgtagaaaccgaactacccgtgtccagtatgttctcgtcattctctgccaactaccctgctgtgggatcaggcgaaattgtaaaagatagaatattattcccaatttacggaggtataaagcagacttcaccagagtttaccgaattagtgaaatatggactctttgtgtcaacacctacaactgtatgtcagagtagctggacttatgaccaggtaaaagcagcgtataggccagattacatatcaggccggttctgggcacaagtgatactcagctgcgctcttgatgcagtcgacttatcaagttgtattgtaaagattatgaatagcagcacagtgatgatggcagcagaaggaaggataataaagatagggattgattacttttactatcagcggtcatcttcttggtggccattggcatttgttacaaaactagacccgcaagagttagcagacacaaactcgatatggctgaccaattccataccaatcccacaatcaaagttccctcggccttcatattcagaaaattattgcacaaagccagcagtttgccctgctacttgtgtcactggtgtatactctgatatttggcccttgacctcatcttcatcactcccgagcataatttggatcggccagtaccttgatgcccctgttggaaggacttatcccagatttggaattgcaaatcaatcacactggtaccttcaagaagatattctacccacctccactgcaagtgcgtattcaaccactacatgttttaagaatactgccaggaatagagtgttctgcgtcaccattgctgaatttgcagatgggttgtttggagagtacaggataacacctcagttgtatgaattagtgagaaataattgaatcacgataattttgggactcatttaattgcagagtgaaattgtcatcttaggaaataatcaattccatgatttttattgaacatgatcaagcaatcatgtgggaaatttattatcacataacttctaatagttttaaatgacgaattaagaaaaaatggagggcgacctctacacaaacatggatgtaaaacaagttgacctaataatacaacccgaggttcatctcgattcacccatcatattgaataaactggcactattatggcgcttgagtggtttacccatgcctgcagacttacgacaaaaatccgtagtgatgcacatcccagaccacatcttagaaaaatcagaatatcggatcaagcaccgtctagggaaaatcaagagtgacatagcacattactgtcagtattttaatattaatttggcaaatcttgatccgataacccaccccaaaagtttgtattggttatccagactaacaatagctagtgctggaacctttagacatatgaaagatagaatcttatgtacagttggctccgaattcggacacaaaattcaagatttattttcactgctgagccataaattagtaggtaacggtgatttatttaatcaaagtctctcaggtacacgtttgactgcgagtccgttatcccctttatgcaatcaatttgtctctgacatcaagtctgcagtcacgacaccctggtcagaagctcgttggtcttggcttcatatcaaacaaacaatgagatacctgataaaacaatcacgcactacaaattcagctcatttaacagaaattataaaagaggaatggggtttagtaggtattactccagatcttgtcattctttttgacagagtcaataatagtctaactgcattaacatttgagatggttctaatgtattcagatgtattagaatcccgtgacaatattgtgctagtggggcgattatctacttttctgcagccagtagttagtagactggaggtgttgtttgatctagtagattcattggcaaaaaccttaggtgacacaatatacgaaattattgcggtgttagagagcttgtcttatgggtccgttcaactacatgatgcaagtcactctcatgcagggtctttcttttcatttaacatgaatgaacttgataacacactatcaaagagggtggatccgaaacacaagaacaccataatgagcattataagacaatgcttttctaatctagatgttgatcaagctgcagagatgctatgcctgatgagattatttggacacccaatgttaactgcaccggatgcagcagccaaagtaaggaaagcaatgtgtgctccaaaacttgttgaacatgacaccatcttgcagacattatccttcttcaagggaataattataaatgggtacagaagatcacactctggcctgtggcccaatgtagagccgtcttcaatctatgatgatgatctcagacagctgtacttagagtcagcagagatttcccatcatttcatgcttaaaaactacaagagtttgagcatgatagaattcaagaagagcatagactacgatcttcacgacgacttaagtactttcttaaaggatagagcaatttgccggccaaaatcccagtgggatgttatattccgtaagtctttacgcagatcccacacgcggtcccagtatatggacgaaattaagagcaaccgattgctaattgattttcttgattctgctgattttgaccctgaaaaggaatttgcatatgtaaccacaatggattatttgcacgataatgaattttgtgcttcatattctctaaaggaaaaggagatcaaaactaccgggaggatatttgcaaaaatgacacgcaatatgagaagttgccaagtgatacttgaatctctgttatcaaaacatatatgcaagttcttcaaagagaacggcgtttcgatggagcaattgtcattgaccaagagtctacttgcaatgtctcaactctcaccaaaagtctcgactctgcaggacactgcatcacgtcatgtaggcaactcaaaatctcagatcgcaaccagcaacccatctcggcatcactcaacaaccaatcagatgtcactctcaaatcggaaaacggttgtagcaactttcttaacaactgatttggaaaaatactgcctgcagtggcgatactcgactattaagttgtttgcacaagctctaaatcaactctttgggattgatcacggatttgaatggatacatttaagactcatgaacagcaccttatttgtcggtgatccttactcgcctcctgaagatccaacactagaggatatagataaagcaccaaatgacgatatcttcatagtttctccaaggggaggcatagagggtttatgtcagaagatgtggaccatgatatcaattagtgcgatacactgtgtagcagagaaaattggtgcacgagtggcagcaatggtgcagggtgataatcaagtaatagctatcaccaaagaactattcagaggagagaaagcctgtgatgtcagagatgagttagacgagctcggtcaggtgttttttgatgagttcaagaggcacaattatgcaattggacacaaccttaagctaaatgagacaatacaaagccaatccttttttgtatattccaaacgaatattctttgaagggcgattgcttagtcaagtcctcaaaaatgctgccaagttatgtatggttgctgaccatctaggtgaaaacacagtatcttcctgtagcaacctgagctctacaattgcccggttggtggaaaatgggtttgagaaggacactgcttttgtgttgaacctagtctacatcatgactcaaattctttttgatgagcattactcgattgtatgcgatcacaatagtgtcaaaagcttgatcggatcaaaaaactatcggaatctattgtactcatctctaataccaggtcagctcggtggtttcaacttcctcaatataagtcggttgttcactaggaatataggtgacccagtaacatgtagtctgtctgatctcaaatgcttcatagccgcaggtctccttccaccctatgtacttaaaaatgtggttctgcgtgagcctggtcctgggacatggttgacgttgtgctctgatccttacacccttaacataccatacacacagctaccaaccacatatctcaaaaagcacacccagcgatcgttgctttcacgtgcagtaaatcctttattagcaggtgtacaagtgccaaatcagcatgaggaagaagagatgttggctcgctttctccttgatcgtgaatatgtgatgccccgcgttgctcatgtaacactagaaacatcggtccttggcaaacggaaacaaatccaaggcttaattgatacaactccaactatcattagaacatctctagtcaatctaccagtgtctaggaagaaatgcgaaaaaataatcaattattctctcaattatattgctgagtgtcatgactccttacttagtcagatctgcttcagtgataataaggaatacttgtggtccacctccttaatatcagttgagacctgtagtgtgacaattgcggactatttgagagctgtcagctggtctaatatattagggggaagaagcatatccggggtgactacacctgatactattgaattaattcaaggttgtttaataggtgaaaattccagttgtactctttgtgaatcgcatgacgacgcattcacatggatgcacttgcctggcccactttacatccctgaaccatcagttactaactctaaaatgcgtgtgccatatctgggttcaaaaacagaggagcgtaaaacagcttcaatggcagcaataaaaggaatgtcacatcacctgcgtgcagtcttaagaggtacatccgtatttatttgggcatctggggacacagatattaattgggataatgcattgcagattgcccaatcacggtgtaacatcacattggatcaaatgagattacttacaccaattcctagcagttcaaatatccaacgtagactcgatgacggaatcagcacgcagaaatttactcctgcaagccttgctcgaatcacatcctctgttcacatctgtaatgacagccaaaggttagagaaggatggctcctctgtcgactcaaacttgatttaccagcaaattatgttacttggactcagcatctttgaaacaatgtactcaatggaccaaaagtgggtattcaataaccataccttacatttgcacactggacactcctgttgtccaagggaactagacataagtttagtgaacccgccaagacatcagaccccggagctgactagcacaacaaccaacccgttcctatatgatcagctcccactaaatcaggataatctgacaacacttgagattaagacattcaaatttaatgagctcaacattgatggtttagattttggtgaaggaatacaattattgagtcgttgtactgcaagattaatggcagaatgtattctagaggagggaataggctcgtcagttaaaaatgaagcaattgtcaattttgataattcagtcaattggatttcagagtgcctaatgtgtgatattcgctcactttgtgttaatttaggtcaagagatactatgtagcctggcataccaaatgtattacttgcgaatcaggggtagaagggccattcttaattacttggacacaactttgcaaaggatccctgtgatacagttagccaacattgcactcaccatttcacaccctgagatatttcgcagaattgtcaacaccgggatccataaccagattaagggcccatatgtggcaacaacagatttcatagctgcaagtagagatatcatattatcaggtgcaagggagtatctatcttatctaagcagtggacaggaagactgttacacattcttcaactgtcaagatggggatcttactccaaaaatggaacagtatcttgcaaggagggcatgccttttaacattactgtataatactgggcaccagatccccattatccgatcactgacaccaatagagaagtgcaaggtgctcacagaatacaatcaacaaattgagtatgcagatcaagagtttagctctgtattgaaagtggtcaatgcactactacaaaatcctaatatagatgcattggtttcaaatctctacttcaccaccagacgtgttttatcaaacctcagatcatgtgataaggctatatcatatattgaatatttgtacactgaggacttcggagaaaaagaagatacagtacaatatgacatcatgacaacaaacgatatcatacttactcatggtctattcacacagatcgaaatatcttaccaagggagtagtctccataaattcctaactccggataacgcgcctggatcattgatcccattctctatttcaccaaattcgcttgcatgtgatcctcttcaccacttactcaagtcggtcggtacatcaagcacaagctggtacaagtatgcaatcgcctatgcagtgtctgaaaagaggtcggctcgattaggagggagcttgtacattggtgaagggagcggaagtgtgatgactttgctagagtatcttgagccatctgttgacatattttacaattcactcttctcaaatggtatgaacccaccacaacgaaattatgggcttatgccactacaatttgtgaattcggtggtttataagaacttaacggctaaatcagaatgtaagctaggatttgtccagcaatttaaaccgttgtggagagacatagacattgagactaatgttacagatccatcatttgtcaattttgcattgaatgaaatcccaatgcaatcattaaaacgagtaaattgtgatgtggaatttgaccgtggtatgccgattgaacgggttattcagggttacactcatatcttacttgttgctacttacggattgcagcaagattcaatactgtgggtgaaagtatataggacatctgaaaaagtatttcagttcttactgagtgccatgatcatgatctttggttatgtcaaaatccacaggaatggttatatgtcggcaaaggatgaggagtacatattgatgtctgactgcaaggaacctgtaaactatacagctgtccctaacattcttacacgtgtaagtgatttagtgtcgaagaatctgagtcttatccatccagaagacctcagaaaggtaaggtgtgaaacagattccctgaatttgaagtgcaatcatatttatgagaaaataattgctagaaaaattccattacaggtgtcatcaactgattctttgctcctccagttaggcggtgtcatcaactcggtgggctcaactgatcctagagaggttgcaacgttatcttccattgagtgtatggactatgttgtctcatcaattgatttggctatattagaggcaaatattgtgatctcagagagtgctgatcttgacctcgctttaatgttaggcccattcaacttgaataagcttaagaaaattgacacaatccttaagtcaagcacctatcagctaatcccgtattggttgcgctatgagtactctattaatccgagatctttgtcatttctaatcactaaattacaacaatgccgaatttcatggtcagatatgataacaatctctgaattttgcaagaaatccaagcggcctatatttattaaacgagtaatagggaatcaacggctgaaatcattctttaatgaaagctcaagtattgttttgacccgggctgaagtcaaagtctgtataaagttcctcggtgcgatcatcaagttgaaataatttctgtgttttttaaggggtatagtattctaagttgcacttgaagtaatatagcttgtaatcattcgctaggggatagaataattcctataatctctgaatatatatctctaggttataacaaatatatacataataaaattgattttaagaaaaaatccgactttcaaagaagattggtgcctgtaatattcttcttgccagatgattatggagggtctagcctaacttaaaacaatcgtattcgatagggaagaatgacatataaagtaactaataaaaaattgtattagtgaaaattaccgtatttcctgtattccatttctggt

SEQ ID NO: 12 - DNA, 15438 bp, Avian paramyxovirus 9
accaaacaaagaaattgtaagatacgttaaagaccgaagtagcaactgacttcgtacgggtagaaggattgaatctcgagtgcgaacacgacgctgtgattcgaaggtccgtactaccatcatgtcctctatattcaatgagtatgagagtctgcttgaaagtcaactcaaaccgacgggctcgaacgtcttaggagagaaaggtgacactccaaaagtcgagatccctgtatttgtgctcaacagtgacaaccctgaagatcgctggaactttactaccttctgtctcagagtcgctgtgagcgaggatgctaataggcctttgcgtcagggggcactcatctctctactttgcgctcattctcaggtgatgaagaatcatgtggccatagcaggaaagcaggatgaggctctgattgtagttctagagattgatactattaatgatggtgttccagccttcaacaataggagcggtgtcacagaggaacgagctcagcgtttcgctatgatagctcaagcattaccccgtgcttgtgcaaatgggacaccgttcaccgtccaagatgcagaagatgatccagtcgaagacataacagacgcccttgatcgcatattgtcaatccaggcgcaagtatgggtgaccgtcgcaaaatccatgacagcgtacgagactgcagatgaatcagaacagaagcgattgaccaagtatgttcagcaaggtcgagtgcagaagaaatgcatgatctaccctgtatgtcggagcatgctgcagcagatcataaggcaatctttagcagtccgacggttcattgtcagtgagctgaaacgagctcggaatacagcaggaggaacatccacgtattataacttcgttgctgatgtagattcctacattaggaatgctgggttaactgcattcttcttgacccttaagtatggtgtgaatacaaagacttctgtccttgcccttagcagcttggcaggcgatcttcaaactgtcaaacagttgatgcggctgtataaagccaaaggagatgatgcaccatacatgactatactgggagacggagaccagatgagatttgcacctgctgaatacgcacagctatactcatacgctatgggaatggcatcagtcatagacaaagggacctcaaggtatcagtacgctcgtgacttcctaaaccccagcttctggaggctgggagtggagtatgcccagactcaaggaagcaacatcaacgaagagatggcatcagaactgaaactcagcccaatagctagaaggatgctgaccactgccgtcacaaaagtagcaaccggagcgtctgattattcggtacctcagcatacagcaggagtcctaactggcttgaattcaacagacggcaaccttgggtctcagaagctgcccacctcaattcagcaggatcagaatgatgatactgccatgttgaacttcatgagggccgtagcacaaggaatgaaggagacaccaattcaggctcctcccacccctggattcggatctcaacaggccgcagacgacgatgactcgcgggatcaagcagactcctgggggctctaatgaaatacggaggttgactccagcccaaacgaacctctagcaactcctaatccctcatccacctacaaactccacatctacatgaccaatccgctcacacaacacggcggaagacaccatccatccccaactgtcccaacccgaagaacatcctcaacttagcccgctaatttcacgaaccattacaaaaaacttatcaacagaaaaaactacgggtagaactgtctgccactgcgagaaagcaaacgcatcaacgcagtcagcactcatcgcagctctccatcacaccaattctagctcaggcacacgcctccagagagaaccatggcatccttcacagacgacgagatatcagatctgatggaacaaagtggtcttgtaatagatgagatcatgacatcccaagggatgcctaaagagaccctagggcgaagtgcaatcccaccagggaaaactcaggccctaactgatgcctgggagaaacacaacaagtcacagagatccaatgcggatcacagcaccggatcaaataacaaaactgatgtcaacacaccccacaatgctgagccgccacaatccaccggcgatccctccgcatctccagaaatggacggcgacacaaccccactcccaaagcaggaaaccgccgaaaagcacccctgcaaagaaggggccactggagggctgctggatatgcttgaccggattgctgccaagcaggatagagctaaaaaagggctcaatccgagatcacaagacacgggcaccctgcactcaggccaattccctacgcagacgcaagacccgacatcccgccgatcaaccaactcatcgggacacagcatggagtccagaacgcccgcccagctgccaatcccgaggagagacgacagcccgcatcaggtaagaagagaggaggagggcatcgcagagaacacagcatggtctggaatgcaaacgggattgtcaccatcagctggtgcaacccagtttgctctccagtcacctacgaaccaagagaattcacatgttcatgcgggagctgccctacagaatgccgactttgtgcaggctctcatagggatattagaaagcattcagcagagagtgagtaaaatggaatatcagatggatttagtcctgcgtcacctgtctagtatgccagccattcgaaatgacattcaacaagttaagaccgctatggcagtgcttgaggccaacattgggatgatgaaaatccttgaccctggatcagcacatatttcttcgctcaatgatcttcgagcagttgcaaggtatcatccagtccttgtagcaggccccggtgaccccaataaaacaattgctgatgataaaaccatcactgtcaatcggctctcccagccggtaactgatcagcgcagcttggtaagagaactcacacccccttccggtgatttcgaggcagaaaaatgcgcaatcaaggcgttattagctgcgagaccactacatccatcggctgcaaaacgaatgtctgataggttagatgcagccaagacatgtgaagaattgaggaaggtgaagagacagattctgaataactgacccaaatagtgtggtttccgccaatgatcaagcgtgatccgccttggacaacttttttgccgatcttaaggagagacaaatcaatttacaccgatctaaaatatcatcagacaccctcaaatcaagaaaacatagatgacagtctgcttgactcatctcttgcatctgatgctatcaattgccctaaaataccacctgacataaataccagattatctctagacctccttggttgttaagaaaaaaaagtaagtacgggtagaaacaggactcaaccgacctaccaccatggatgcttctaggatgatcagtctatatgtagaccccactagcagttctagttcaatactcgcattcccaatagtcatggaagccacaggagacggacgaaagcaaatttcaccccaatatcgcattcagagattagatcactggtcagacagcagtcgagatgcagtattcatcaccacatatgggtttatatttggataccctaaatcacgtgctgatcgaggccagcttaatgaagaaattaggcctgtgctgctctctgctgcaacgctatgtctgggcagtgtggcgaatactggagatcaggttgcaattgctcgggcatgcttgtcactacaaatatcttgcaaaaagagtgctactagtgaggagaaaatgatatttgcaatcacccaagctccgcagattttacaatcatgtcgtgctgtttcgcaaaaattcgtctccgttggatcaaataaatgtgtgaaagcacctgaaagaatcgagggaggccagcagtatgactataaggtcaacttcgtgtctctcactatagtaccaaaagatgacgtatatagggtcccaaaacctgtcctatcagtcagcagtcccactctattccgccttgccctgagtgttaacatcgcaatcgacatcaatgccgacaatcctttgtctaagacgcttattaagaccgaaagcggctttgaagcaaatttgttcctgcatgtgggtattctctcaaacattgacaagcggggaaagaaggtgacgttcgagaagttagagaagaaaatccggcggatggaactgactgcaggattaagtgatatgtttggtccgtccatcatcctgaaggccaaagggccgaggacaaagttgatgtcagcattcttttctaatacgggaacagcgtgttatccgatcgcacaagcatctcctccagtatcgaagatcttgtggagccaaagcggacacctccaggaggttaagatacttgtacaatcgggaacctcgaaaatgattgcattaacagccgatcaagaaatcacaacaacaaagctcgatcagcacgccaagattcaatcatttaacccattcaaaaagtaagttgcatggctcacgaatagctcaggtcttcttgccttaaaatcagccaatgaatatgtgataggatattcagtgtctcgaatcattaccgatcaaaaaaccccattaaatcatacacctgatcattagacaagaggtaatccaaatagcattaaaaaaaatccccaaaagaattaaaactaaaacacagcacgggtagaaagtgagctgtatatcactcaatccacaatctaccatagtgacacaatggggtacttccacctattacttatactaacagcgattgccatatctgcgcacctctgctataccacgacattggatggtagaaaactgcttggtgcaggcatagtgataacagaagagaagcaagttagggtgtacacagctgcgcaatcaggaacaattgtcttaaggtctttccgtgtggtctccttagacagatactcgtgcatggaatccactattgagtcatataacaagactgtatataacatacttgcacctctgggcgatgcaatccgccgaatacaggcaagtggtgtatcggttgagcgtatccgagagggccgcatatttggtgccatccttgggggagttgccttaggtgtagccaccgcagcacagataacagctgcaattgctttgattcaggctaacgagaacgcaaaaaacatcctgcgtattaaagacagtataactaagaccaacgaggcagtgagagatgtaactaatggcgtgtcgcagttaactatcgctgtaggtaaattacaggacttcgtcaataaggaattcaataagacaactgaggccattaattgtgtacaggcagctcaacaattaggtgtggagctaagcctctatctgaccgagatcactacagtcttcggacctcagataacctctcctgctttaagcaaattgactatccaagcgctgtataatttggcgggcgtaagcttggatgtactactgggaaggctcggagcagacaattcacagttatcatctttggttagtagtggtcttattaccggacagcccattctctacgactcggaatctcaaatattggcactgcaagtgtcactaccctccattagtgacttaaggggagtgagagcgacatacttagacacgttggctgtcaacactgcagcaggacttgcatctgctatgattccaaaggtagtaatccaatctaataatatagttgaagaattagatactacagcatgtatagcagcagaagctgacttatactgtacgaggattactacattccccattgcgtcggctgtatcagcctgcattcttggggatgtatcgcaatgcctttattcaaagactaatggcgtcttaaccactccatatgtagcagtaaaggggaaaattgtagccaattgtaagcatgtcacatgtaggtgtgtagatcctacatccatcatatctcaaaattacggtgaagcagcgactcttatcgatgatcagctatgcaaggtaatcaacttagatggtgtgtccatacagctgagcggcacatttgaatcgacttatgtgcgcaacgtctcgataagtgcaaacaaggtcattgtctcaagcagtatagatatatctaatgagctggagaatgttaacagctctttaagttcggctctggaaaaactggatgaaagtgacgctgcgctaagcaaagtaaatgttcacttaactagcacctcagctatggccacatacattgttctaactgtaattgctcttatcttggggtttgtcggcctaggattgggttgctttgctatgataaaagtaaagtctcaagcaaagacactactatggcttggtgcacatgctgaccgatcatatatactccagagtaagccggctcaatcgtccacataatacaacaacaatcaatcctgactatcatataatacatgaatcatttcttcttccgattataaaaaaataagaaacctaattaggccaatacgggtagaacaggcttccaccccgtatttcttcggctgtgatcctgtacctgagttcttcccaccaacaccaggacctctcctaaattgcatcaccatggaatcaggaatcagccaggcatctcttgtcaatgacaacatagaattaaggaatacgtggcgcacggccttccgtgtggtctccttattactcggcttcaccagcttggtgctcactgcttgcgctttacacttcgctttgaatgccgctacccctgcggatctctctagtatcccagtcgctgttgaccaaagtcatcatgaaattctacaaaccttgagtctgatgagcgacattggcaataagatttacaagcaggtagcactagatagtccagtggcgctgctcaacactgaatcaaccttaatgagcgcaattacatcactatcttatcagattaacaatgcagcgaataactcaggttgtggcgcccctgtgcatgataaggattttatcaatggagtggcaaaggaattatttgtagggtctcaatacaatgcctcgaactatcgaccctccaggttccttgagcatctaaatttcatccccgcccctactacgggaaaaggttgcaccagaattccgtcctttgatctagctgcaacacattggtgttatactcacaatgtgattcttaatggttgtaatgatcatgctcaatcttatcaatacatatccctcgggatactcaaggtgtcagccacgggaaacgtgttcttatctactctcagatctatcaacctggatgatgatgaaaaccggaaatcatgtagcatatcagcaacgccactagggtgtgacttactttgtgctaaagtcactgagagagaagaggcagattacaattcagatgcagcgacgagattagttcatggcaggttaggttttgatggggtataccatgagcaggccctgcctgtagaatcattgttcagtgactgggttgcaaactatccgtcagtcggcggaggcagttactttgataatagggtatggtttggcgtgtatggggggatcagacctggctctcagactgatctgctccagtctgagaagtacgcgatatatcgtaggtacaataatacctgccctgataataatcccacccagattgagcgggccaaatcatcttatcgtccgcagcggtttggccagcggcttgtacaacaagcaattctatcaattagagtggagccatctttgggtaatgatcctaaactatctgtgttagataatacagtcgtgttgatgggggcggaagcaaggataatgacatttggccacgtggcattaatgtatcaaagagggtcatcatattttccttctgcactattataccctctcagtttaacaaatggtagtgcagcagcatccaagcctttcatattcgagcaatatacaaggccaggtagcccaccttgtcaggccactgcaagatgtccaaattcatgtgttactggtgtctacacagacgcatacccgttattttggtctgaagatcataaagtgaatggtgtatatggtatgatgttagatgacatcacatcacggttaaacccggtagcagctatatttgataggtatggtaggagtagagtgactagggttagcagtagcagcacgaaggcagcttacactacaaatacatgctttaaggttgtcaaaacaaagagagtatactgcttgagcattgccgagatagagaatacactgtttggagaattcagaataacccctttactctccgagataatatttgacccaaaccttgaaccctcagacacgagccgtaactgaggaaaatccgttctggcagacagtggttggatagaccttgcgtcgatagccctcactgttggcactgcgtcgtccctatattcaaacaccacattagcggagtatacagatagtcggccatgatgaatcaaatgtcatgcgatttgagcataaccgaagcagaatcaggatatacccggctctaccatatcagggagaacagctggtaagctgtaatcctcaataatcctaaaaactgcaggtaatacaaaaggatcagcctatagggagcttcaacaatcgttagaaaaaaacgggtagaacatggataatccaggacaatctcgccctgatcatcaagtgattctacccgaagcgcatctttcctcaccgatcgtaaggcataagttatattatttctggagactaacaggagtaccactaccccactcagcagaatttgatacgctagtcctatccagaccatggaacaaaatattgcagagcaactcgccagaagtactgaggatgaagcggctaggtgcgaacgtccacgcgactctagatcactctcgaccaataaaggctttgatccacccggagactttagcatggctaactgatctgtctataggggtatctatctctagatttagaggaatagaaaagaaagtatctcgcctgctccatgacaatagagagaaattttgtacacttgtttctcagattcatgaaggattgttcggtggtgtaggaggggttcggaataatctgtcaccagagtttgaaagtttgctcaatggaactaacttctggtttggcgggaaatattcaaacacaaaattcacttggcttcacattaaacaattgcagagacatcttatactcacagcgcgtatgagatctgggcagcaactttacatccaattaaagcatacaaggggttatgtccatataactccagagttaactatgattacatgcaacggaaaaaaccttgttacagcacttacacctgagatggtcttaatgtatagtgacatgctagaaggaagagatatggtcataagtgttgcacagcttgtgaatggcctgaatgtcctagcagataggattgagtgtcttcttgacttgattgaccaattggcgtgcttgataaaggatgctatatatgaaataattgggattttggagggtttagcttatgcagcagtccagctgctggagccgtccggaaaattcgcaggggatttctttgaattcaatctcagagagatagctgccatattgcgagaacacatagaccctgtgttagctaacagggtacttgagtctattacctggatttacagtggtctgacagacaaccaagcagcagagatgctctgtatcctccgcttgtggggccaccctacattagagtccagaacagctgcagctgcagtgcgaaagcaaatgtgcgcgccaaaactcattgacttcgacatgatccaacaagtattggctttctttaaagggacaatcatcaatggatatagaagacaaaactcaggagtctggccaagagttaaaaaggatactatctatggatcaacactccaacagttgcatgctgactatgcagagatatcacacgaattaatgctgaaagaatacaagcgtctagcaatgcttgagtttgagaagtgtattgacatagacccagtatccaatttaagcatgttcttgaaggacaaggctatagcacacacgcgaccaaattggctggcatcttttaaaagaactttgttatccgatagacagcagctcttagcaaaggatgcaacttcgaccaatcgtctgctgatagaattcctagaatctagcaactttgacccatatcaggagatgacctatttgacaagtcttgaatttcttagagataatgacgtggcagtatcatattcgttaaaggagaaagaagttaagcccaatggtagaatcttcgcaaagcttaccaaacgactcagaaattgtcaggtgatggcagagaatatcctagcagacgaaattgcaccttttttccaagggaatggagtcattcaaagcagcatctctctgacgaaaagtatgttagcaatgagtcaactgtcatttaattgcaacagattctcgatcggaaaccgcagagaagggatcaaagagaataggacacgacaccgtgaacgaaagcgaagaaggcgagtagctacatatatcacaactgacctgcagaagtactgtctcaattggaggtatcagaccatcaagccttttgcccatgcgattaatcagctgacagggcttgatttgttttttgagtggatccaccttcgtctaatggataccactatgttcgttggagatccatacaacccaccctctgatccaacaattgaaaacctggatgatgcacccaatgatgatatctttattgtaagcggaagaggagggatcgagggattatgtcaaaagctttggactaccatatcaatatccgcaatacaattagcagccacccggtcaaagtgtagggtagcctgtatggtgcaaggtgacaatcaggtgatcgcagtgacccgagaagtaaatccagatgactcagaagatgcggtcttagatgaattacataaggccagcgacagattctttgaggaactcactcacgtgaatcatctgatcggacataacctgaaagatagagagaccatacgctcagatacttgttttatctatagcaagcgagtattcaaggatggtaagatactttctcaggccctcaagaatgctgcaaagctcgtcttaatatctggggagattggggagaacactcctatgtcatgcgggaatattgcttctacagtgtctcgtctgtgtgaaaatgggctgcccaaagatgcctgctatatgatcaattatatattaacctgtatacaatttttctttgacaatgagttttccattgtccccgcttctcagcgtggatccacagttgaatgggtggataacctttcatttgtacacgcgtatgcactgtggccaggccaatttggaggattgaacaacttacaatattctagattgtttactcgcaatatcggggacccatgcactactgcacttgcagagattaagagattagagagagctcaactaataccagggaagctaatcaagaacttgcttgctaggaagccaagcaatggaacatgggcgtctctttgtaatgatccttattcactcaatattgaaacagcaccaagcccaaatctcatcctcaagaaacatactcagagagtactatttgaatcctgcaccaatcccctattacaaggggtttatagtgaagaaaatgatacggaagaagcagaattagcagaattcttgctcaatcaagaagctatacatccgcgcgtggcacacgttataatggaggccagcgcagtcggtagaaagaagcaaattcagggactaatcgatacaactaacaccatcataaagattgcacttgggcggcgtcctcttggtgcaaggaggttaaggaagataaacagttattcttctatgcacatgttgatcttcctggatgatatattcctacctaaccatcctccatctcccttcgtctcctcagtgatgtgttctgttgccctagcggattacctacgtcagattacctggttgcctctgacaaatggtaggaagatattaggtgtaaataatccagatacccttgagttagtatcaggatcgatgctgaatctaaacggatattgtgacttatgtaatagtggagataaccaatttacgtggttccatctcccagcagatatagagctagcggacagttcatcatccaaccctccaatgcgtataccttatgtgggatccaagacccaggaaaggagaaatgcatcaatggccaagattagcaacatgtcccctcatatgaaggcagcattgagattggcgtctgtgaaggtaagggcttacggtgataatgagcataattggcaagttgcatggcagctagcaaatactcgatgtgcgatatcccttgaacatctaaaacttctagcccctctaccaactgcagggaaccttcagcatcgattggatgatagcataacccagatgacctttactcccgcttctctctatcgggtggcaccttatatccacatctccaatgactcacaaagaatgttttctgatgagggggttaaggagagcaacatcatctatcagcagataatgttattgggtctatcagctatcgaatcattgttccccttgaccactaatcatgtatatgaagaagtgacactacaccttcatactcaattcagctgctgcctgagagaggcggcccttgcggtcccatttgagctccagggcaaagtacctaggattcgtgctgctgaggggaaccaattcgtgtatgactcatccccacttttggaacctgaggctcttcaactcgatgtggctactttcaagaactatgagttggacttagaccattattcaacgatagacttgatgcatgtacttgaggttacgtgtggaaagctaataggtcagtcggtgatttcatacaatgaggacacttctataaagaatgatgcaattattgtatacgataatacccggaattggatcagtgaggcccaaaattgtgacctggtgaagttatttgagtatgctgcactagaaatcttgctggactgcgcattccaaatgtattatctaagggttcgcggatacaagaacatcctaatatacatggcagacctaattcgtaatatgcccggtatattgctctctaatattgctgccacaatctcccatcccattatccatactagactatacaatgcagggttgctggatcatgggagtgcgcaccaacttgcaagcattgattttattgaattatcagctaatttattggtaacatgtatagctcgtgtatgtactacacttctatccggtgaaaccctgatgcttgcatttccatccgttctagacgagaatttgacggagaaaatgtttcttctaatcgctcgatactgctctttgttagcgttgttgtactcatctaaggttcctataccaaatattaggggcctgactgccgaagataagtgccggatgctcacaaatcatctcatgaaccttccatctgaatttcggctgaccgaaaatcaggtacgaaatgtactgcaaccagcactgacaactttcccagcaaacctctattatatgtcaagaaagagtcttaatatcatcagagagagggagataaagatgctattattcaaatgttgttccctgccggggatgaagctacaagcacggtggcagttaatttgggatacgaaagtaaatgaccccattgttaagtggcgacgcattgaattcttatgcgagctcgatctctctggtcaggcaaggtttggagtcatactggatgaatgcatctctgatgttgataaaaacggacagggcatcctcgactttgtcccaatgactcgatacctattcaggggtgtaggccaggcatcctcatcatggtataaagctgccaatttattgtcacttcctgaagtgcgccaggcacgtttcggtaactcattgtacttagcagaaggtagcggtgcaataatgagtctgttagagctccacgtaccacatgagaagatttactacaatactctcttttataacgagatgaaccccccgcaaagacatttcggcccaacgccaactcaattccttgcatcggtcgtttacaagaaccttcaggcaggtatagtctgcaaagatgggtatgttcaggagttctgccctttatggagagacgttgccgatgaaagtgatcttgcttcagataggtgtgtctcattcattacatcagaggtgcctggaggcactgtatctctactccattgtgacatagaaacaaccctggaaccaagctgggcttacttggagcaattagccactaatatctctctaatcgggatgcacgtcctgcgagagaatggagtgttcatcatcaaagtactatacacccagagtttcttttttcatctattgctggcaatcttagctccttgtagtaaaaggatacggatcatatccaatggatactcagtacggggagattttgagtgctacctagtcgcgacaatcagttatacaggggggcatgtcttcatgcaagaggtgatccgctctgccaaggcgttagttagagggggcggtagtatcatgacaaaacaagatgaacaacaattgaatcttgctttccagaggcagctcaacaggattcgtgggatactgggacagaggatatcgataatgatacgctacttgcagcatactattgatatggcattgattgaagcgggaggccaacctgtaagaccgagcaatgttggaatcaacaaggcactcgacttaggagatgagacatatgaggaaatcatgatacagcatattgacacaacacttaagacagcaatcttcctagaacaagaagaagaactggcagacacagtctttgtgttaacaccttataacctaacggcaagaggaaaatgtaatacagtacttattgcatgcactaaacatctatttgaaacaactatattacagactacacgagacgacatggataagatagagaaattgttgtcccttatcttacaaggtcatatctcgcttcaggatctcctgccactcaagtcatatcttaaacgtagcaattgtcccaagtacctcctcgattcactaggacgtatcaggctaaaagaggtatttgaacactcatcccgcatggtactaaccagaccgatgcaaaagatgtatctcaaatgtctcggaaatgctattaagggataccttgcagtggatgcatctcattgcaattgaatcatgacgcaatctcttttatacatcatactcgtaatcaatcatagttaccatcatttttaagaaaaacagtaacgatttatggtgtcacgtatgttgccaaatctttgtttggt

SEQ ID NO: 13 - DNA, 15185 bp, Artificial Sequence
  Description of Artificial Sequence Synthetic polynucleotide
accaaacagagaatccgtgagttacgataaaaggcgaaagagcaattgaagtcacacgggtagaaggtgtgaatctcgagtgcgagcccgaagcacaaactcgagaaagccttctgccaacatgtcctccgtatttgatgagtacgaacagctcctcgcggctcagactcgccccaacggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaataataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccattactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatcggatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggtcgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcagacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatccctgctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggtgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacccgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactgccccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgccctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaaaactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaacccgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttccacgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggtaaataatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagtcgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaagacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagctcaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagaccacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagcagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcaaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaaattgacactgcgctgattgaagccgggggacagcccgtccgtccattctgtgcggagagtctggtgagcacgctagcgaacataactcagataacccagatcatcgctagccacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt

SEQ ID NO: 14 - DNA, 16696 bp, Artificial Sequence
  Description of Artificial Sequence Synthetic polynucleotide
acgaaaaagaagaataaaaggcagaagccttttaaaaggaaccctgggctgtcgtaggtgtgggaaggttgtattccgagtgcgcctccgaggcatctactctacacctatcacaatggctggtgtcttctcccagtatgagaggtttgtggacaatcaatcccaagtgtcaaggaaggatcatcggtccttagcaggaggatgccttaaagttaacatccctatgcttgtcactgcatctgaagaccccaccactcgttggcaactagcatgcttatctctaaggctcctgatctccaactcatcaaccagtgctatccgtcagggggcaatactgactctcatgtcattaccatcacaaaacatgagagcaacagcagctattgctggttccacaaatgcagctgttatcaacaccatggaagtcttaagtgtcaacgactggaccccatccttcgaccctaggagcggtctttctgaggaagatgctcaagttttcagagacatggcaagagatctgccccctcagttcacctctggatcacccttcacatcagcattggcggaggggttcactcctgaagatactcatgacctgatggaggccttgaccagtgtgctgatacagatctggatcctggtggctaaggccatgaccaacattgacggctctggggaggccaatgaaagacgtcttgcaaagtacatccaaaaaggacagcttaatcgtcagtttgcaattggtaatcctgcccgtctgataatccaacagacaatcaaaagctccttaactgtccgtaggttcttggtctctgagcttcgtgcgtcacgaggtgcagtaaaagaaggatccccttactatgcagctgttggggatatccacgcttacatctttaatgcgggattgacaccattcttgaccaccttaagatacgggataggcaccaagtacgccgctgttgcactcagtgtgttcgctgcagatattgcaaagttgaagagcctacttaccctgtaccaggacaagggtgtagaagctggatacatggcactccttgaggatccagactccatgcactttgcacctggaaacttcccacacatgtactcctatgcaatgggggtagcttcttaccatgatcctagcatgcgccaataccaatacgccaggaggttcctcagccgtcctttctacttactaggaagggacatggccgccaagaacacaggcacgctggatgagcaactggcgaaggaactgcaagtatcagagagagatcgcgccgcattatccgctgcgattcaatcagcgatggaggggggagagtccgacgacttcccactgtcgggatccatgccggctctctctgagaatgcgcaaccagttacccccagacctcaacagtcccagctctctcccccccaatcatcaaacatgccccaatcagcacccaggaccccagactatcaacccgactttgaactgtaggcttcatcaccgcaccaacaacagcccaagaagaccacccctccccccacacatctcacccagccacccataaagactcagtcccacgccccagcatctccttcatttaattaaaaaccgaccaacagggtggggaaggagagtcattggctactgccaattgtgtgcagcaatggattttactgacattgatgctgtcaactcattgatcgaatcatcatcggcaatcatagactccatacagcatggagggctgcaaccagcgggcaccgtcggcctatcgcagatcccaaaagggataaccagcgcattaaccaaggcctgggaggctgaggcggcaactgccggtaatggggacacccaacacaaatctgacagtccggaggatcatcaggccaacgacacagattcccctgaagacacaggtactgaccagaccacccaggaggccaacatcgttgagacaccccaccccgaggtgctgtcagcagccaaagccagactcaagaggcccaaagcagggagggacacccgcgacaactcccctgcgcaacccgatcatcttttaaaggggggcctcctgagcccacaaccagcagcatcatgggtgcaaaatccacccagtcatggaggtcccggcaccgccgatccccgcccatcacaaactcaggatcattcccccaccggagagaaatggcgattgtcaccgacaaagcaaccggagacattgaactggtggagtggtgcaacccggggtgcacagcagtccgaattgaacccaccagactcgactgtgtatgcggacactgccccaccatctgtagcctctgcatgtatgacgactgatcaggtacaactactaatgaaggaggttgctgacataaaatcactccttcaggcgttagtgaggaacctcgctgtcttgccccaattgaggaatgaggttgcagcaatcagaacatcacaggccatgatagaggggacactcaattcgatcaagattcttgaccctgggaattatcaggaatcatcactaaacagttggttcaaacctcgccaagatcacactgttgttgtgtctggaccagggaatccattggccatgccaaccccagtccaagacaacaccatattcctggacgagctagccagacctcatcctagtgtggtcaatccttccccacccatcaccaacaccaatgttgaccttggcccacagaagcaggctgcaatagcctatatctccgctaaatgcaaggatccggggaaacgagatcagctatcaaggctcattgagcgagcaaccaccccaagtgagatcaacaaagttaaaagacaagcccttgggctctagatcactcgatcacccctcatggtgatcacaacaataatcagaacccttccgaaccacatgaccaacccagcccaccgcccacaccgtccatcacgcgtgtagctgatttattcaaaaccgccaccatgtgccatcagcagctggtcatctcatggttctccctggtgtttctggcctcacctctggtcgcaatctgggaactgaaaaaggatgtgtacgtggtggagctggactggtatcccgatgcccctggcgagatggtggtgctgacctgcgacacacccgaggaggatggcatcacctggacactggatcagagctccgaggtgctgggaagcggcaagaccctgacaatccaggtgaaggagttcggcgacgccggccagtacacctgtcacaagggaggagaggtgctgagccactccctgctgctgctgcacaagaaggaggatggcatctggtccacagacatcctgaaggatcagaaggagccaaagaacaagaccttcctgcggtgcgaggccaagaattatagcggccggttcacctgttggtggctgaccacaatctccaccgatctgacattttctgtgaagtctagcaggggatcctctgacccacagggagtgacatgcggagcagccaccctgagcgccgagagggtgcgcggcgataacaaggagtacgagtattccgtggagtgccaggaggactctgcctgtccagcagcagaggagtccctgcctatcgaagtgatggtggatgccgtgcacaagctgaagtacgagaattataccagctccttctttatccgggacatcatcaagcccgatccccctaagaacctgcagctgaagcctctgaagaatagcagacaggtggaggtgtcctgggagtaccctgacacctggagcacaccacactcctatttctctctgaccttttgcgtgcaggtgcagggcaagtccaagcgggagaagaaggacagagtgttcaccgataagacatctgccaccgtgatctgtagaaagaacgcctctatcagcgtgagggcccaggaccgctactattctagctcctggtccgagtgggcctctgtgccttgcagcggcggaggaggaggaggatctaggaatctgccagtggcaacccctgacccaggcatgttcccctgcctgcaccacagccagaacctgctgagggccgtgtccaatatgctgcagaaggcccgccagacactggagttttacccttgtaccagcgaggagatcgaccacgaggacatcacaaaggataagacctccacagtggaggcctgcctgccactggagctgaccaagaacgagtcctgtctgaacagccgggagacaagcttcatcaccaacggctcctgcctggcctctagaaagacaagctttatgatggccctgtgcctgtctagcatctacgaggacctgaagatgtatcaggtggagttcaagaccatgaacgccaagctgctgatggaccccaagaggcagatctttctggatcagaatatgctggccgtgatcgacgagctgatgcaggccctgaacttcaatagcgagacagtgcctcagaagtcctctctggaggagccagatttctacaagaccaagatcaagctgtgcatcctgctgcacgcctttcggatcagagccgtgacaatcgaccgcgtgatgtcctatctgaatgcttcctaatgacccacgcgtcatcccttgccaaacatcctgccgtagctgatttattcaaaagagctcatttgatatgacctggtaatcataaaatagggtggggaaggtgctttgcctgtaagggggctccctcatcttcagacacgtgcccgccatctcaccaacagtgcaatggcagacatggacacggtgtatatcaatctgatggcagatgacccaacccaccaaaaagaactgctgtcctttcctctcatccctgtgaccggtcctgacgggaagaaggaactccaacaccagatccggacccaatccttgctcgcctcagacaaacaaactgaacggttcatcttcctcaacacttacggattcatctatgacaccacaccggacaagacaactttttccaccccagagcatattaatcagcctaagaggacgacggtgagtgccgcgatgatgaccattggcctggttcccgccaatatacccctgaacgaactaacggctactgtgttcagccttaaagtaagagtgaggaaaagtgcgaggtatcgggaagtggtctggtatcaatgcaatccagtaccggccctgcttgcagccaccaggtttggtcgccaaggaggtctcgagtcgagcactggagtcagtgtaaaggctcccgagaagatagattgtgagaaggattatacctactacccttatttcttatctgtgtgctacatcgccacctccaacctgttcaaggtaccgaggatggttgctaatgcaaccaacagtcaattataccaccttaccatgcaggtcacatttgcctttccaaaaaacatccctccagccaaccagaaactcctgacacaggtggatgagggattcgagggcactgtggattgccattttgggaacatgctgaaaaaggatcggaaagggaacatgaggacactgtcccaggcggcagataaggtcagacgaatgaatattcttgttggtatctttgacttgcatgggccaacgctcttcctggagtataccgggaaactgacaaaggctctgctagggttcatgtccaccagccgaacagcaatcatccccatatctcagctcaatcccatgctgagtcaactcatgtggagcagtgatgcccagatagtaaagttaagggttgtcataactacatccaaacgcggcccgtgcgggggtgagcaggagtatgtgctggatcccaaattcacagttaagaaagaaaaggctcgactcaaccctttcgagaaggcagcctaatgatttaatccgcaagatcccagaaatcagaccactctatactatccactgatcactggaaatgtaattgtacagttgatgaatctgtgaagaatcaattaaaaaaccggatccttattagggtggggaagtagttgattgggtgtctaaacaaaagcatttcttcacacctccccgccacgaaacaaccacaatgaggctatcaaacacaatcttgaccttgattctcatcatacttaccggctatttgataggtgtccactccaccgatgtgaatgagaaaccaaagtccgaagggattaggggtgatcttacaccaggtgcgggtattttcgtaactcaagtccgacagctccagatctaccaacagtctgggtaccatgatcttgtcatcagattgttacctcttctaccaacagagcttaatgattgtcaaagggaagttgtcacagagtacaataacactgtatcacagctgttgcagcctatcaaaaccaacctggatactttgttggcagatggtagcacaagggatgttgatatacagccgcgattcattggggcaataatagccacaggtgccctggctgtagcaacggtagctgaggtaactgcagctcaagcactatctcagtcaaaaacgaatgctcaaaatattctcaagttgagagatagtattcaggccaccaaccaagcagtttttgaaatttcacagggactcgaagcaactgcaaccgtgctatcaaaactgcaaactgagctcaatgagaatatcatcccaagtctgaacaacttgtcctgtgctgccatggggaatcgccttggtgtatcactctcactctatttgaccttaatgaccactctatttggggaccagatcacaaacccagtgctgacgccaatctcttacagcaccctatcggcaatggcgggtggtcacattggtccagtgatgagtaagatattagccggatctgtcacaagtcagttgggggcagaacaactgattgctagtggcttaatacagtcacaggtagtaggttatgattcccagtatcagctgttggttatcagggtcaaccttgtacggattcaggaagtccagaatactagggttgtatcactaagaacactagcagtcaatagggatggtggactttacagagcccaggtgccacccgaggtagttgagcgatctggcattgcagagcggttttatgcagatgattgtgttctaactacaactgattacatctgctcatcgatccgatcttctcggcttaatccagagttagtcaagtgtctcagtggggcacttgattcatgcacatttgagagggaaagtgcattactgtcaactcccttctttgtatacaacaaggcagtcgtcgcaaattgtaaagcagcgacatgtagatgtaataaaccgccatctatcattgcccaatactctgcatcagctctagtaaccatcaccaccgacacttgtgctgaccttgaaattgagggttatcgtttcaacatacagactgaatccaactcatgggttgcaccaaacttcacggtctcaacctcacaaatagtatcggttgatccaatagacatatcctctgacattgccaaaattaacaattctatcgaggctgcgcgagagcagctggaactgagcaaccagatcctttcccgaatcaacccacggattgtgaacgacgaatcactaatagctattatcgtgacaattgttgtgcttagtctccttgtaattggtcttattattgttctcggtgtgatgtacaagaatcttaagaaagtccaacgagctcaagctgctatgatgatgcagcaaatgagctcatcacagcctgtgaccaccaaattggggacacccttctaggtgaataatcatatcaatccattcaataatgagcgggacataccaatcaccaacgactgtgtcacaaggccggttaggaatgcaccggatctctctccttcctttttaattaaaaacggttgaactgagggtgagggggggggtgtgcatggtagggtggggaaggtagccaattcctgcccattgggccgaccgtaccaagagaagtcaacagaagtatagatgcagggcgacatggagggtagccgtgataacctcacagtagatgatgaattaaagacaacatggaggttagcttatagagttgtatccctcctattgatggtgagtgccttgataatctctatagtaatcctgacgagagataacagccaaagcataatcacggcgatcaaccagtcgtatgacgcagactcaaagtggcaaacagggatagaagggaaaatcacctcaatcatgactgatacgctcgataccaggaatgcagctcttctccacattccactccagctcaatacacttgaggcaaacctgttgtccgccctcggaggttacacgggaattggccccggagatctagagcactgtcgttatccggttcatgactccgcttacctgcatggagtcaatcgattactcatcaatcaaacagctgactacacagcagaaggccccctggatcatgtgaacttcattccggcaccagttacgactactggatgcacaaggatcccatccttttctgtatcatcatccatttggtgctatacacacaatgtgattgaaacaggttgcaatgaccactcaggtagtaatcaatatatcagtatgggggtgattaagagggctggcaacggcttaccttacttctcaacagtcgtgagtaagtatctgaccgatgggttgaatagaaaaagctgttccgtagctgcgggatccgggcattgttacctcctttgtagcctagtgtcagagcccgaacctgatgactatgtgtcaccagatcccacaccgatgaggttaggggtgctaacaagggatgggtcttacactgaacaggtggtacccgaaagaatatttaagaacatatggagcgcaaactaccctggggtagggtcaggtgctatagcaggaaataaggtgttattcccattttacggcggagtgaagaatggatcaacccctgaggtgatgaataggggaagatattactacatccaggatccaaatgactattgccctgacccgctgcaagatcagatcttaagggcagaacaatcgtattatcctactcgatttggtaggaggatggtaatgcagggagtcctaacatgtccagtatccaacaattcaacaatagccagccaatgccaatcttactatttcaacaactcattaggattcatcggggcggaatctaggatctattacctcaatggtaacatttacctttatcaaagaagctcgagctggtggcctcacccccaaatttacctacttgattccaggattgcaagtccgggtacgcagaacattgactcaggcgttaacctcaagatgttaaatgttactgtcattacacgaccatcatctggcttttgtaatagtcagtcaagatgccctaatgactgcttattcggggtttattcagatgtctggcctcttagccttacctcagacagcatatttgcatttacaatgtacttacaagggaagacgacacgtattgacccagcttgggcgctattctccaatcatgtaattgggcatgaggctcgtttgttcaacaaggaggttagtgctgcttattctaccaccacttgtttttcggacaccatccaaaaccaggtgtattgtctgagtatacttgaagtcagaagtgagctcttgggggcattcaagatagtgccattcctctatcgtgtcttataggcacctgcttggtcaagaaccctgagcagccataaaattaacacttgatcttccttaaaaacacctatctaaattactgtctgagatccctgattagttaccctttcaatcaatcaattaatttttaattaaaaacggaaaaatgggcctagttccaaggaaaggatgggacccattagggtggggaaggattactttgttccttgactcgcacccacgtacacccaatcccattcctgtcaagaaggaacccttcccaaactcaccttgcaatgtccaatcaggcagctgagattatactacccaccttccatcttttatcacccttgatcgagaataagtgcttctactacatgcaattacttggtctcgtgttaccacatgatcactggagatggagggcattcgtcaattttacagtggatcaagcacaccttaaaaatcgtaatccccgcttaatggcccacatcgatcacactaaggatagactaagggctcatggtgtcttgggtttccaccagactcagacaagtgagagccgtttccgtgtcttgctccatcctgaaactttaccttggctatcagcaatgggaggatgcatcaaccaggttcccaaggcatggcggaacactctgaaatctatcgagcacagtgtgaagcaggaggcgactcaactgaagttactcatggaaaaaacctcactaaagctaacaggagtatcttacttattctccaattgcaatcccgggaaaactgcagcgggaactatgcccgtactaagtgagatggcatcagaactcttgtcaaatcccatctcccaattccaatcaacatgggggtgtgctgcttcagggtggcaccatgtagtcagcatcatgaggctccaacagtatcaaagaaggacaggtaaggaagagaaagcaatcactgaagttcagtatggctcggacacctgtctcattaatgcagactacaccgtcgttttttccgcacaggaccgtgtcatagcagtcttgcctttcgatgttgtcctcatgatgcaagacctgcttgaatcccgacggaatgtcttgttctgtgcccgctttatgtatcccagaagccaactacatgagaggataagtacaatactggcccttggagaccaactcgggagaaaagcaccccaagtcctgtatgatttcgtagctaccctcgaatcatttgcatacgctgctgtccaacttcatgacaacaaccctatctacggtggggctttctttgagttcaatatccaagaactggaagctattttgtcccctgcacttaataaggatcaagtcaacttctacataagtcaagttgtctcagcatacagtaaccttcccccatctgaatcagcagaattgctatgcttactacgcctgtggggtcatcccttgctaaacagtcttgatgcagcaaagaaagtcagagaatctatgtgtgctgggaaggttcttgattataatgctattcgactagttttgtctttttatcatacgttattaatcaatgggtatcggaagaaacataagggtcgctggccaaatgtgaatcaacattcactactcaacccgatagtgaagcagctttactttgatcaggaggagatcccacactctgttgcccttgagcactatttagatatctcgatgatagaatttgagaagacttttgaagtggaactatctgatagtctaagcatctttctgaaggataagtcgatagctttggataaacaagaatggcacagtggttttgtctcagaagtgactccaaagcacctacgaatgtctcgtcatgatcgcaagtctaccaataggctattgttagcctttattaactcccctgaattcgatgttaaggaagagcttaaatatttgactacaggtgagtatgccactgacccaaatttcaatgtctcttactcactgaaagagaaggaagttaagaaagaagggcgcattttcgcaaagatgtcacagaaaatgagagcatgccaggttatttgtgaagagttactagcacatcatgtggctcctttgtttaaagagaatggtgttacacaatcggagctatccctgacaaagaatttgttggctattagccaactgagttacaactcgatggccgctaaggtgcgattgctgaggccaggggacaagttcaccgctgcacactatatgaccacagacctaaaaaagtactgccttaactggcggcaccagtcagtcaaattgttcgccagaagcctggatcgactatttgggttagaccatgctttttcttggatacacgtccgtctcaccaatagcactatgtacgttgctgacccattcaatccaccagactcagatgcatgcacaaatttagacgacaataagaacactgggatttttattataagtgctcgaggtggtatagaaggccttcaacagaaactatggactggcatatcaattgcaatcgcccaggcggcagcagccctcgagggcttacgaattgctgccactttgcagggggataaccaggttttagcgattacgaaagaattcatgaccccagtctcggaggatgtaatccacgagcagctatctgaagcgatgtcgcgatacaagaggactttcacataccttaattatttaatggggcaccaattgaaggataaagaaaccatccaatccagtgacttcttcgtttactccaaaaggatcttcttcaatgggtcaatcctaagtcaatgcctcaagaacttcagtaaactcactaccaatgccactacccttgctgagaacactgtagccggctgcagtgacatctcctcatgcatagcccgttgtgtggaaaacgggttgcctaaggatgctgcatatgttcagaatataatcatgactcggcttcaactgttgctagatcactactattctatgcatggtggcataaactcagagttagagcagccaactctaagtatccctgtccgaaacgcaacctatttaccatctcaattaggcggttacaatcatttgaatatgacccgactattctgtcgcaatatcggtgacccgcttactagttcttgggcagagtcaaaaagactaatggatgttggccttctcagtcgtaagttcttagaggggatattatggagacccccgggaagtgggacattttcaacactcatgcttgatccgttcgcacttaacattgattacttaaggccaccagagacaataatccgaaaacacacccaaaaagtcttgttgcaggattgtcctaatcctctattagcaggtgtagttgacccgaactacaaccaggaattagaattattagctcagttcctgcttgatcgggaaaccgttattcccagggctgcccatgccatctttgaactgtctgtcttgggaaggaaaaaacatatacaaggattggttgatactacaaaaacaattattcagtgctcattagaaagacagccactgtcctggaggaaagttgagaacattgtaacctacaatgcgcagtatttcctcggggccacccagcaggttgacaccaatatctcagaaaggcagtgggtgatgccaggtaatttcaagaagcttgtatctcttgacgattgctcagtcacgttgtccactgtgtcacggcgcatttcttgggccaatctacttaactggagggctatagatggtttggaaactccagatgtgatagagagtattgatggccgccttgtgcaatcatccaatcaatgcggcctatgtaatcaaggattgggctcctactcctggttcttcttgccctccgggtgtgtgttcgaccgtccacaagattctcgagtggttccaaagatgccatacgtgggatccaaaacggatgagagacagactgcgtcagtgcaggctatacagggatccacatgtcaccttagagcagcattgagacttgtatcactctacctttgggcctatggagattctgacatatcatggctagaagccgcgacattggctcaaacacggtgcaatatttctcttgatgacctgcggatcctgagccctcttccttcctcggcaaatttacaccacagattgaatgacggggtaacacaagtgaaattcatgcccgccacatcgagccgggtgtcaaagttcgtccaaatttgcaatgacaaccagaatcttatccgtgatgatgggagtgttgattccaatatgatttatcagcaggttatgatattagggcttggagagattgaatgtttgttagctgacccaatcgatacaaacccagaacaactgattcttcacctacactctgataattcttgctgtctccgggagatgccaacgaccggttttgtacctgctttaggattgaccccatgcttaactgtcccaaagcacaatccgtatatttatgatgatagcccaatacccggtgatttggatcagaggctcattcaaaccaaattctttatgggttctgacaatctagataatcttgatatctaccagcagcgagctttactgagtcggtgtgtggcttatgacattatccaatcagtattcgcttgcgatgcaccagtatctcagaagaatgatgcaatccttcacactgactaccatgaaaattggatctcagagttccgatggggtgaccctcgcataatccaagtaacagcaggttacgagttaattctgttccttgcataccagctttattatctcagagtgaggggtgaccgtgcaatcctgtgttatattgataggatactcaacaggatggtatcttccaatctaggcagtctcatccagacgctctctcatccggagattaggaggagattttcattgagtgatcaagggttccttgtcgaaagggagctagagccaggtaagccactggtaaaacaagcggttatgttcctaagggactcagtccgctgcgctttagcaactatcaaggcaggaattgagcctgagatctcccgaggtggctgtacccaggatgagctgagctttacccttaagcacttactatgtcggcgtctctgtataattgctctcatgcattcggaagcaaagaacttggtcaaagttagaaaccttccagtagaggaaaaaaccgccttactataccagatgttgatcactgaggccaatgccaggagatcagggtctgctagtatcatcataagcttagtttcagcaccccagtgggacattcatacaccagcgttgtattttgtatcaaagaaaatgctggggatgctcaaaaggtcaaccacacccttggatataagtgacctttctgagagccagaacctcacaccaacagaattgaatgatgttcctggtcacatggcagaggaatttccctgtttgtttagcagttataacgctacatatgaagacacaattacttacaatccaatgactgaaaaactcgcagtgcacttggacaatggttccaccccttccagagcgcttggtcgtcactacatcctgcgaccccttgggctttactcgtctgcatggtaccggtctgcagcactattagcgtcaggggccctcagtgggttgcctgaggggtcaagcctgtacttgggagaggggtatgggaccaccatgactctacttgagcccgttgtcaagtcctcaactgtttactaccatacattgtttgacccaacccggaatccttcacagcggaactacaaaccagaaccgcgggtattcactgattccatttggtacaaggatgatttcacacgaccacctggtggcattgtaaatctatggggtgaagacgtacgtcagagtgatattacacagaaagacacggttaatttcatattatctcgggtcccgccaaaatcactcaaattgatacacgttgatattgagttctccccagactctgatgtacggacgctactatctggctattcccattgtgcactattggcctactggctactgcaacctggagggcgatttgcggttagagttttcttaagtgaccatatcatagtcaacttggtcactgccattctgtccgcttttgactctaatctggtgtgcattgcgtcaggattgacacacaaggatgatggggcaggttatatttgtgcaaagaagcttgcaaatgttgaggcttcaagaattgagtattacttgaggatggtccacggctgtgttgactcattaaaaattcctcatcaattaggaatcattaaatgggctgagggtgaagtgtcccgacttaccaaaaaggcggatgatgaaataaactggcggttaggtgatccagttaccagatcatttgatccggtttctgagctaataattgcgcgaacagggggatcagtattaatggaatacgggacttttactaacctcaggtgtgcgaacttggcagatacatataaacttttggcttcaattgtagagaccaccttaatggaaataagggttgagcaagatcagttggaagatgattcgaggagacaaatccaggtagtccctgcttttaatacaagatccgggggaaggatccgtacattgattgagtgtgctcagctgcaggtcatagatgttatctgtgtgaacatagatcacctctttcccaaacaccgacatgctcttgtcacacaacttacttaccagtcagtgtgccttggggacttgattgaaggcccccaaattaagacatatctaagggccaggaagtggatccaacgtaggggactcaatgagacaattaaccatatcatcactggacaagtgtcgcggaataaggcaagggattttttcaagaggcgcctgaagttggttggcttttcgctctgtggcggttggggctacctctcactttagctgcttagattgttgattattatgaataatcggagtcgaaatcgtaaatagaaagacataaaattgcaaataagcaatgatcgtattaatatttaataaaaaatatgtcttttatttcgt

SEQ ID NO: 15 - DNA, 5026 bp, Homo sapiens
agttccctatcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaatgtaagtatatttcctttcttactaaaattattacatttagtaatctagctggagatcatttcttaataacaatgcattatactttcttagaattacaagaatcccaaactcaccaggatgctcacatttaagttttacatgcccaagaaggtaagtacaatattttatgttcaatttctgttttaataaaattcaaagtaatatgaaaatttgcacagatgggactaatagcagctcatctgaggtaaagagtaactttaatttgtttttttgaaaacccaagtttgataatgaagcctctattaaaacagttttacctatatttttaatatatatttgtgtgttggtgggggtgggaagaaaacataaaaataatattctcactttatcgataagacaattctaaacaaaaatgttcatttatggtttcatttaaaaatgtaaaactctaaaatatttgattatgtcattttagtatgtaaaataccaaaatctatttccaaggagcccacttttaaaaatcttttcttgttttaggaaaggtttctaagtgagaggcagcataacactaatagcacagagtctggggccagatatctgaagtgaaatctcagctctgccatgtcctagctttcatgatctttggcaaattacctactctgtttgtgattcagtttcatgtctacttaaatgaataactgtatatacttaatatggctttgtgagaattagtaagtaaatgtaaagcactcagaaccgtgtctggcataaggtaaataccatacaagcattagctattattagtagtattaaagataaaattttcactgagaaatacaaagtaaaattttggactttatctttttaccaatagaacttgagatttataatgctatatgacttattttccaagattaaaagcttcattaggttgtttttggattcagatagagcataagcataatcatccaagctcctaggctacattaggtgtgtaaagctacctagtagctgtgccagttaagagagaatgaacaaaatctggtgccagaaagagcttgtgccagggtgaatccaagcccagaaaataataggatttaaggggacacagatgcaatcccattgactcaaattctattaattcaagagaaatctgcttctaactacccttctgaaagatgtaaaggagacagcttacagatgttactctagtttaatcagagccacataatgcaactccagcaacataaagatactagatgctgttttctgaagaaaatttctccacattgttcatgccaaaaacttaaacccgaatttgtagaatttgtagtggtgaattgaaagcgcaatagatggacatatcaggggattggtattgtcttgacctacctttcccactaaagagtgttagaaagatgagattatgtgcataatttagggggtggtagaattcatggaaatctaagtttgaaaccaaaagtaatgataaactctattcatttgttcatttaaccctcattgcacatttacaaaagattttagaaactaataaaaatatttgattccaaggatgctatgttaatgctataatgagaaagaaatgaaatctaattctggctctacctacttatgtggtcaaattctgagatttagtgtgcttatttataaagtggagatgatacttcactgcctacttcaaaagatgactgtgagaagtaaatgggcctattttggagaaaattcttttaaattgtaatataccatagaaatatgaaatattatatataatatagaatcaagaggcctgtccaaaagtcctcccaaagtattataattttttatttcactgggacaaacatttttaaaatgcatcttaatgtagtgattgtagaaaagtaaaaatttaagacatatttaaaaatgtgtcttgctcaaggctatattgagagccactactacatgattattgttacctagtgtaaaatgttgggattgtgatagatggcatccaagagttccttctctctcaacattctgtgattcttaactcttagactatcaaatattataatcatagaatgtgatttttatgcttccacattctaactcatctggttctaatgattttctatgcagattggaaaagtaatcagcctacatctgtaataggcatttagatgcagaaagtctaacattttgcaaagccaaattaagctaaaaccagtgagtcaactatcacttaacgctagtcataggtacttgagccctagtttttccagttttataatgtaaactctactggtccatctttacagtgacattgagaacagagagaatggtaaaaactacatactgctactccaaataaaataaattggaaattaatttctgattctgacctctatgtaaactgagctgatgataattattattctaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaaggtaaggcattactttatttgctctcctggaaataaaaaaaaaaaagtagggggaaaagtaccacattttaaagtgacataacatttttggtatttgtaaagtacccatgcatgtaattagcctacattttaagtacactgtgaacatgaatcatttctaatgttaaatgattaactggggagtataagctactgagtttgcacctaccatctactaatggacaagcctcatcccaaactccatcacctttcatattaacacaaaactgggagtgagagaaggtactgagttgagtttcacagaaagcaggcagattttactatatatttttcaattccttcagatcatttactggaatagccaatactgattacctgaaaggcttttcaaatggtgtttccttatcatttgatggaaggactacccataagagatttgtcttaaaaaaaaaaactggagccattaaaatggccagtggactaaacaaacaacaatctttttagaggcaatccccactttcagaatcttaagtatttttaaatgcacaggaagcataaaatatgcaagggactcaggtgatgtaaaagagattcacttttgtctttttatatcccgtctcctaaggtataaaattcatgagttaataggtatcctaaataagcagcataagtatagtagtaaaagacattcctaaaagtaactccagttgtgtccaaatgaatcacttattagtggactgtttcagttgaattaaaaaaatacattgagatcaatgtcatctagacattgacagattcagttccttatctatggcaagagttttactctaaaataattaacatcagaaaactcattcttaactcttgatacaaatttaagacaaaaccatgcaaaaatctgaaaactgtgtttcaaaagccaaacactttttaaaataaaaaaatcccaagatatgacaatatttaaacaattatgcttaagaggatacagaacactgcaacagttttttaaaagagaatacttatttaaagggaacactctatctcacctgcttttgttcccagggtaggaatcacttcaaatttgaaaagctctcttttaaatctcactatatatcaaaatatttcctccttagcttatcaactagaggaagcgtttaaatagctcctttcagcagagaagcctaatttctaaaaagccagtccacagaacaaaatttctaatgtttaaacttttaaaagttggcaaattcacctgcattgatactatgatggggtagggataggtgtaagtatttatgaagatgttcttcacacaaatttatcccaaacagaagcatgtcctagcttactctagtgtagttctgttctgctttggggaaaatataaggagattcacttaagtagaaaaataggagactctaatcaagatttagaaaagaagaaagtataatgtgcatatcaattcatacatttaacttacacaaatataggtgtacattcagaggaaaagcgatcaagtttatttcacatccagcatttaatatttgtctagatctatttttatttaaatctttatttgcacccaatttagggaaaaaatttttgtgttcattgactgaattaacaaatgaggaaaatctcagcttctgtgttactatcatttggtatcataacaaaatatgtaattttggcattcattttgatcatttcaagaaaatgtgaataattaatatgtttggtaagcttgaaaataaaggcaacaggcctataagacttcaattgggaataactgtatataaggtaaactactctgtactttaaaaaattaacatttttcttttatagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaa

SEQ ID NO: 16 - DNA, 1600 bp, Artificial Sequence
  Description of Artificial Sequence Synthetic polynucleotide
atgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggcggcggatctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagactcgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaat

SEQ ID NO: 17 - DNA, 1602 bp, Artificial Sequence
  Description of Artificial Sequence Synthetic polynucleotide
atgtgccatcagcagctggtcatctcatggttctccctggtgtttctggcctcacctctggtcgcaatctgggaactgaaaaaggatgtgtacgtggtggagctggactggtatcccgatgcccctggcgagatggtggtgctgacctgcgacacacccgaggaggatggcatcacctggacactggatcagagctccgaggtgctgggaagcggcaagaccctgacaatccaggtgaaggagttcggcgacgccggccagtacacctgtcacaagggaggagaggtgctgagccactccctgctgctgctgcacaagaaggaggatggcatctggtccacagacatcctgaaggatcagaaggagccaaagaacaagaccttcctgcggtgcgaggccaagaattatagcggccggttcacctgttggtggctgaccacaatctccaccgatctgacattttctgtgaagtctagcaggggatcctctgacccacagggagtgacatgcggagcagccaccctgagcgccgagagggtgcgcggcgataacaaggagtacgagtattccgtggagtgccaggaggactctgcctgtccagcagcagaggagtccctgcctatcgaagtgatggtggatgccgtgcacaagctgaagtacgagaattataccagctccttctttatccgggacatcatcaagcccgatccccctaagaacctgcagctgaagcctctgaagaatagcagacaggtggaggtgtcctgggagtaccctgacacctggagcacaccacactcctatttctctctgaccttttgcgtgcaggtgcagggcaagtccaagcgggagaagaaggacagagtgttcaccgataagacatctgccaccgtgatctgtagaaagaacgcctctatcagcgtgagggcccaggaccgctactattctagctcctggtccgagtgggcctctgtgccttgcagcggcggaggaggaggaggatctaggaatctgccagtggcaacccctgacccaggcatgttcccctgcctgcaccacagccagaacctgctgagggccgtgtccaatatgctgcagaaggcccgccagacactggagttttacccttgtaccagcgaggagatcgaccacgaggacatcacaaaggataagacctccacagtggaggcctgcctgccactggagctgaccaagaacgagtcctgtctgaacagccgggagacaagcttcatcaccaacggctcctgcctggcctctagaaagacaagctttatgatggccctgtgcctgtctagcatctacgaggacctgaagatgtatcaggtggagttcaagaccatgaacgccaagctgctgatggaccccaagaggcagatctttctggatcagaatatgctggccgtgatcgacgagctgatgcaggccctgaacttcaatagcgagacagtgcctcagaagtcctctctggaggagccagatttctacaagaccaagatcaagctgtgcatcctgctgcacgcctttcggatcagagccgtgacaatcgaccgcgtgatgtcctatctgaatgcttcctaatga

SEQ ID NO: 18 - DNA, 903 bp, Artificial Sequence
  Description of Artificial Sequence Synthetic polynucleotide
atggcgccgcgccgcgcgcgcggctgccgcaccctgggcctgccggcgctgctgctgctgctgctgctgcgcccgccggcgacccgcggcgattataaagatgatgatgataaaattgaaggccgcattacctgcccgccgccgatgagcgtggaacatgcggatatttgggtgaaaagctatagcctgtatagccgcgaacgctatatttgcaacagcggctttaaacgcaaagcgggcaccagcagcctgaccgaatgcgtgctgaacaaagcgaccaacgtggcgcattggaccaccccgagcctgaaatgcattcgcgatccggcgctggtgcatcagcgcccggcgccgccgagcggcggcagcggcggcggcggcagcggcggcggcagcggcggcggcggcagcctgcagatgcgcattagcaaaccgcatctgcgcagcattagcattcagtgctatctgtgcctgctgctgaacagccattttctgaccgaagcgggcattcatgtgtttattctgggctgctttagcgcgggcctgccgaaaaccgaagcgaactgggtgaacgtgattagcgatctgaaaaaaattgaagatctgattcagagcatgcatattgatgcgaccctgtataccgaaagcgatgtgcatccgagctgcaaagtgaccgcgatgaaatgctttctgctggaactgcaggtgattagcctggaaagcggcgatgcgagcattcatgataccgtggaaaacctgattattctggcgaacaacagcctgagcagcaacggcaacgtgaccgaaagcggctgcaaagaatgcgaagaactggaagaaaaaaacattaaagaatttctgcagagctttgtgcatattgtgcagatgtttattaacaccagc

SEQ ID NO: 19 - DNA, 477 bp, Human papillomavirus type 16
atgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 20 - DNA, 297 bp, Human papillomavirus type 16
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataa

SEQ ID NO: 21 - DNA, 3043 bp, Homo sapiens
ttctcagagtggctgcagtctcgctgctggatgtgcacatggtggtcattccctctgctcacaggggcaggggtccccccttactggactgaggttgccccctgctccaggtcctgggtgggagcccatgtgaactgtcagtggggcaggtctgtgagagctcccctcacactcaagtctctctcacagtggccagagaagaggaaggctggagtcagaatgaggcaccagggcgggcatagcctgcccaaaggcccctgggattacaggcaggatggggagccctatctaagtgtctcccacgccccaccccagccattccaggccaggaagtccaaactgtgcccctcagagggagggggcagcctcaggcccattcagactgcccagggagggctggagagccctcaggaaggcgggtgggtgggctgtcggttcttggaaaggttcattaatgaaaacccccaagcctgaccacctagggaaaaggctcaccgttcccatgtgtggctgataagggccaggagattccacagttcaggtagttcccccgcctccctggcattttgtggtcaccattaatcatttcctctgtgtatttaagagctcttttgccagtgagcccagctacacagagagaaaggctaaagttctctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatggtaagtgagagaatgtgggcctgtgctaggcaccagtggccctgactggccacgcctgtcagcttgataacatgacattttccttttctacagaatgaaacagtagaagtcatctcagaaatgtttgacctccaggtaagatgcttctctctgacatagctttccagaagcccctgccctggggtggaggtggggactccattttagatggcaccacacagggttgtccactttctctccagtcagctggctgcaggaggagggggtagcaactgggtgctcaagaggctgctggccgtgcccctatggcagtcacatgagctcctttatcagctgagcggccatgggcagacctagcattcaatggccaggagtcaccaggggacaggtggtaaagtgggggtcacttcatgagacaggagctgtgggtttggggcgctcactgtgccccgagaccaagtcctgttgagacagtgctgactacagagaggcacagaggggtttcaggaacaacccttgcccacccagcaggtccaggtgaggccccacccccctctccctgaatgatggggtgagagtcacctccttccctaaggctgggctcctctccaggtgccgctgagggtggcctgggcggggcagtgagaagggcaggttcgtgcctgccatggacagggcagggtctatgactggacccagcctgtgcccctcccaagccctactcctgggggctgggggcagcagcaaaaaggagtggtggagagttcttgtaccactgtgggcacttggccactgctcaccgacgaacgacattttccacaggagccgacctgcctacagacccgcctggagctgtacaagcagggcctgcggggcagcctcaccaagctcaagggccccttgaccatgatggccagccactacaagcagcactgccctccaaccccggtgagtgcctacggcagggcctccagcaggaatgtcttaatctagggggtggggtcgacatggggagagatctatggctgtggctgttcaggaccccagggggtttctgtgccaacagttatgtaatgattagccctccagagaggaggcagacagcccatttcatcccaaggagtcagagccacagagcgctgaagcccacagtgctccccagcaggagctgctcctatcctggtcattattgtcattacggttaatgaggtcagaggtgagggcaaacccaaggaaacttggggcctgcccaaggcccagaggaagtgcccaggcccaagtgccaccttctggcaggactttcctctggccccacatggggtgcttgaattgcagaggatcaaggaagggaggctacttggaatggacaaggacctcaggcactccttcctgcgggaagggagcaaagtttgtggccttgactccactccttctgggtgcccagagacgacctcagcccagctgccctgctctgccctgggaccaaaaaggcaggcgtttgactgcccagaaggccaacctcaggctggcacttaagtcaggcccttgactctggctgccactggcagagctatgcactccttggggaacacgtgggtggcagcagcgtcacctgacccaggtcagtgggtgtgtcctggagtgggcctcctggcctctgagttctaagaggcagtagagaaacatgctggtgcttccttcccccacgttacccacttgcctggactcaagtgttttttatttttctttttttaaaggaaacttcctgtgcaacccagattatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggcacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatatgcttctacttgtccagtgttctagtttgtttttaaccatgagcaaatgccat

SEQ ID NO: 22 - protein, 5 aa, Artificial Sequence
  Description of Artificial Sequence Synthetic peptide
  MOD_RES (4)..(4): Any amino acid except Pro
VPGXG

SEQ ID NO: 23 - protein, 10 aa, Artificial Sequence
  Description of Artificial Sequence Synthetic peptide
  MOD_RES (4)..(4): Any amino acid except Pro
  MOD_RES (9)..(9): Any amino acid except Pro
VPGXGVPGXG

SEQ ID NO: 24 - protein, 6 aa, Avian paramyxovirus 1
GRQGRL

SEQ ID NO: 25 - protein, 6 aa, Avian paramyxovirus 2
KPASRF

SEQ ID NO: 26 - protein, 6 aa, Avian paramyxovirus 3
RPSGRL

SEQ ID NO: 27 - protein, 6 aa, Avian paramyxovirus 4
DIQPRF

SEQ ID NO: 28 - protein, 6 aa, Avian paramyxovirus 6
KRKKRF

SEQ ID NO: 29 - protein, 6 aa, Avian paramyxovirus 7
LPSSRF

SEQ ID NO: 30 - protein, 6 aa, Avian paramyxovirus 8
YPQTRL

SEQ ID NO: 31 - protein, 6 aa, Avian paramyxovirus 9
IREGRI

SEQ ID NO: 32 - DNA, 6 bp, Artificial Sequence
  Description of Artificial Sequence Synthetic oligonucleotide
acgcgt

SEQ ID NO: 33 - DNA, 8 bp, Artificial Sequence
  Description of Artificial Sequence Synthetic oligonucleotide
ccgccacc

SEQ ID NO: 34 - protein, 532 aa, Artificial Sequence
  Description of Artificial Sequence Synthetic polypeptide
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDSKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 35 - protein, 7 aa, Artificial Sequence
  Description of Artificial Sequence Synthetic peptide
GGGGGGS

SEQ ID NO: 36 - protein, 20 aa, Artificial Sequence
  Description of Artificial Sequence Synthetic peptide
SGGSGGGGSGGGSGGGGSLQ

SEQ ID NO: 37 - protein, 301 aa, Artificial Sequence
  Description of Artificial Sequence Synthetic polypeptide
MAPRRARGCRTLGLPALLLLLLLRPPATRGDYKDDDDKIEGRITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 38 - protein, 12 aa, Artificial Sequence
  Description of Artificial Sequence Synthetic peptide
DYKDDDDKIEGR

SEQ ID NO: 39 - protein, 77 aa, Homo sapiens
ITCPPPMSVEHADIWVKSYSLYSRERYICNSGFKRKAGTSSLTECVLNKATNVAHWTTPSLKCIRDPALVHQRPAPP

SEQ ID NO: 40 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 41 - protein, 30 aa, Artificial Sequence
  Description of Artificial Sequence Synthetic polypeptide
MAPRRARGCRTLGLPALLLLLLLRPPATRG

SEQ ID NO: 42 - DNA, 60 bp, Artificial Sequence
  Description of Artificial Sequence Synthetic oligonucleotide
agcggcggcagcggcggcggcggcagcggcggcggcagcggcggcggcggcagcctgcag

SEQ ID NO: 43 - DNA, 90 bp, Artificial Sequence
  Description of Artificial Sequence Synthetic oligonucleotide
atggcgccgcgccgcgcgcgcggctgccgcaccctgggcctgccggcgctgctgctgctgctgctgctgcgcccgccggcgacccgcggc

SEQ ID NO: 44 - DNA, 36 bp, Artificial Sequence
  Description of Artificial Sequence Synthetic oligonucleotide
gattataaagatgatgatgataaaattgaaggccgc

SEQ ID NO: 45 - DNA, 21 bp, Artificial Sequence
  Description of Artificial Sequence Synthetic oligonucleotide
ggtggcggtggcggcggatct

SEQ ID NO: 46 - protein, 328 aa, Homo sapiens
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 47 - DNA, 984 bp, Homo sapiens
atgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagt

SEQ ID NO: 48 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDSKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 49 - DNA, 594 bp, Homo sapiens
agaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagactcgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 50 - DNA, 231 bp, Homo sapiens
attacctgcccgccgccgatgagcgtggaacatgcggatatttgggtgaaaagctatagcctgtatagccgcgaacgctatatttgcaacagcggctttaaacgcaaagcgggcaccagcagcctgaccgaatgcgtgctgaacaaagcgaccaacgtggcgcattggaccaccccgagcctgaaatgcattcgcgatccggcgctggtgcatcagcgcccggcgccgccg

SEQ ID NO: 51 - DNA, 486 bp, Homo sapiens
atgcgcattagcaaaccgcatctgcgcagcattagcattcagtgctatctgtgcctgctgctgaacagccattttctgaccgaagcgggcattcatgtgtttattctgggctgctttagcgcgggcctgccgaaaaccgaagcgaactgggtgaacgtgattagcgatctgaaaaaaattgaagatctgattcagagcatgcatattgatgcgaccctgtataccgaaagcgatgtgcatccgagctgcaaagtgaccgcgatgaaatgctttctgctggaactgcaggtgattagcctggaaagcggcgatgcgagcattcatgataccgtggaaaacctgattattctggcgaacaacagcctgagcagcaacggcaacgtgaccgaaagcggctgcaaagaatgcgaagaactggaagaaaaaaacattaaagaatttctgcagagctttgtgcatattgtgcagatgtttattaacaccagc

SEQ ID NO: 52 - DNA, 6 bp, Avian paramyxovirus 4
catcga